(19) United States
(12) Patent Application Publication
(10) Pub. No.: US 2025/0370337 A1
(43) Pub. Date: Dec. 4, 2025

(54) ONIUM SALT, CHEMICALLY AMPLIFIED RESIST COMPOSITION, AND PATTERN FORMING PROCESS

(71) Applicant: SHIN-ETSU CHEMICAL CO., LTD., Tokyo (JP)

(72) Inventor: Masahiro Fukushima, Joetsu-shi (JP)

(73) Assignee: SHIN-ETSU CHEMICAL CO., LTD., Tokyo (JP)

(21) Appl. No.: 19/220,694

(22) Filed: May 28, 2025

(30) Foreign Application Priority Data
Jun. 4, 2024 (JP) 2024-090656

(51) Int. Cl. G03F 7/029; G03F 7/039

(52) U.S. Cl. CPC G03F 7/029; G03F 7/0392

(57) ABSTRACT

The chemically amplified resist composition comprises an onium salt as a photoacid generator. The resist composition has a high solvent solubility and a high sensitivity and being improved in lithography properties such as LWR, CDU, MEF, EL, and DOF with high contrast when processed by photolithography using high-energy radiation such as KrF or ArF excimer laser, EB, or EUV; and a pattern forming process using the chemically amplified resist composition. The onium salt has the formula (1):

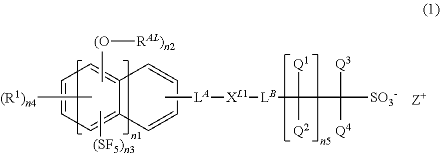

### CROSS-REFERENCE TO RELATED APPLICATION

This non-provisional application claims priority under 35 U.S.C. § 119 (a) on Patent Application No. 2024-090656 filed in Japan on Jun. 4, 2024, the entire contents of which are hereby incorporated by reference.

### TECHNICAL FIELD

The invention relates to an onium salt, a chemically amplified resist composition, and a pattern forming process.

### BACKGROUND ART

In recent years, in accordance with higher integration and operating speed of LSIs, a finer pattern rule has been required, and far ultraviolet lithography and extreme ultraviolet (EUV) lithography are promising as the next generation in microfabrication technology.

Photolithography using an ArF excimer laser (ArF lithography) started partial use from the fabrication of 130-nm node devices and became the main lithography technique for 90-nm node devices. Although lithography using F₂ laser of wavelength 157 nm was initially regarded as a promising lithography technique for a next 45-nm node, its development was retarded by several problems. Therefore, a highlight was suddenly placed on the ArF immersion lithography that introduces a liquid having a higher refractive index than water between the projection lens and the wafer and ethylene glycol, or glycerin in air, allowing the projection lens to be designed to a numerical aperture (NA) of 1.0 or higher and achieving a higher resolution. See Non-Patent Document 1. The immersion lithography requires a resist composition which is substantially insoluble in water.

In ArF lithography, in order to prevent degradation of a precise and expensive optical system material, a highly sensitive resist composition capable of exhibiting sufficient resolution at a small dose of exposure is required. As a method for realizing this, it is most common to select a highly transparent component at a wavelength of 193 nm as a component thereof. For example, as the base polymer, polyacrylic acid and a derivative thereof, a norbornene-maleic anhydride alternating polymer, polynorbornene, a ring-opened metathesis polymer, a ring-opened metathesis polymer hydrogenated product, and the like have been proposed, and some results have been obtained from the viewpoint of increasing the transparency of the resin alone.

Recently, a highlight is put on a negative tone resist adapted for organic solvent development as well as a positive tone resist adapted for alkaline development. Since a very fine hole pattern that cannot be achieved by the positive tone is resolved through negative tone exposure, a negative pattern is formed by developing with an organic solvent using a positive resist composition having high resolution. Studies have also been conducted to double a resolution by combining two developments of alkaline development and organic solvent development. As an ArF resist composition for negative tone development with an organic solvent, positive ArF resist compositions of the prior art design may be used. Such pattern forming processes are described in Patent Documents 1 to 3.

To meet the current rapid progress of microfabrication technology, development efforts are put on not only the process, but also the resist composition. Various studies have also been made on photoacid generators, and sulfonium salts of triphenylsulfonium cations with perfluoroalkanesulfonic acid anions are generally used. These salts generate perfluoroalkanesulfonic acids, especially perfluorooctanesulfonic acid (PFOS), which are considered problematic with respect to their non-degradability, biological concentration, and toxicity. It is rather restricted to apply these salts to the resist composition. Photoacid generators that generate perfluorobutanesulfonic acid are currently used. However, when these salts are used for the resist composition, diffusion of the generated acid is large, and it is difficult to achieve high resolution. For this problem, various partially fluorinated alkane sulfonic acids and salts thereof are developed. For example, Patent Document 1 describes, as the prior art photoacid generators, a photoacid generator capable of generating α,α-difluoroalkanesulfonic acid, specifically, a photoacid generator capable of generating di(4-tert-butylphenyl) iodonium 1,1-difluoro-2-(1-naphthyl) ethanesulfonate or α,α,β,β-tetrafluoroalkanesulfonic acid. Despite a reduced degree of fluorine substitution, the photoacid generators do not have a decomposable substituent such as an ester structure. Thus, the photoacid generators are insufficient from the viewpoint of environmental safety due to ease of decomposition, and there are limitations to the molecular design for changing the size of the alkanesulfonic acid. Fluorine-containing starting reactants are expensive.

As the circuit line width is reduced, the degradation of contrast by acid diffusion becomes more serious for the resist composition. The reason is that the pattern feature size is approaching the diffusion length of acid, resulting in a lowering of mask fidelity and a degradation of pattern rectangularity because a dimensional shift on wafer (mask error factor (MEF)) relative to a dimensional shift on mask is exaggerated. Accordingly, in order to sufficiently gain benefits from a reduction of exposure light wavelength and an increase of NA, the resist material is required to increase a dissolution contrast or restrain acid diffusion, as compared with the prior art materials. One approach is to lower the bake temperature for suppressing acid diffusion and hence, improving MEF. A low bake temperature, however, inevitably leads to a low sensitivity.

Incorporating a bulky substituent or polar group into the photoacid generator is effective for suppressing acid diffusion. Patent Document 4 discloses a photoacid generator capable of generating 2-acyloxy-1,1,3,3,3-pentafluoropropane-1-sulfonic acid which is fully soluble and stable in solvents and allows for a wide span of molecular design. In particular, a photoacid generator having a bulky substituent incorporated therein or capable of generating 2-(1-adamantyloxy)-1,1,3,3,3-pentafluoropropane-1-sulfonic acid is characterized by slow acid diffusion. Patent Documents 5 to 7 describe photoacid generators having fused ring lactone, sultone or thiolactone incorporated as the polar group. Although some improvement in performance is observed due to the acid diffusion suppressing effect of the polar group incorporated, they are still insufficient in precise control of acid diffusion. Their lithography performance is unsatisfactory when evaluated totally in terms of MEF, pattern profile and sensitivity.

Incorporating a polar group into an anion of a photoacid generator is effective for suppressing acid diffusion, but disadvantageous from the viewpoint of solvent solubility. Intending to improve solvent solubility, Patent Documents 8 and 9 propose to incorporate an alicyclic group into a cation moiety of a photoacid generator. Specifically, a cyclohexane ring or adamantane ring is incorporated. While incorporating such an alicyclic group achieves an improvement in solubility, a relatively large number of carbon atoms is necessary to insure a satisfactory solubility. This means that the molecular structure of the photoacid generator becomes bulky, causing to degrade lithography performance factors such as line width roughness (LWR) and critical dimension uniformity (CDU) in forming small-size patterns.

For the purpose of improving the dissolution contrast, an acid labile group is also incorporated in an anion or a cation of a photoacid generator (Patent Documents 10 and 11). Many of them have a structure in which a carboxy group is protected with an acid labile group. A deprotection reaction of a protective group by an acid proceeds before and after exposure, but since a polar group to be generated is a carboxy group, swelling by a developer occurs during alkaline development, and pattern collapse occurs during formation of small-size patterns. Therefore, a photoacid generator having a partial structure in which phenol is protected with an acid labile group and a fluorine atom is introduced in the vicinity in an anion has also been proposed (Patent Document 12). As a result, although the contrast between the exposed region and the unexposed region and the swelling of the developer at the time of alkaline development have been somewhat improved, there is still room for improvement. In order to meet the demand for further miniaturization, it is important to develop a novel photoacid generator, and it is desired to develop a photoacid generator in which acid diffusion is sufficiently controlled, solvent solubility is excellent, and pattern collapse is effectively suppressed.

### CITATION LIST

Patent Document 1: JP-A 2008-281974
Patent Document 2: JP-A 2008-281975
Patent Document 3: JP 4554665
Patent Document 4: JP-A 2007-145797
Patent Document 5: JP 5061484
Patent Document 6: JP-A 2016-147879
Patent Document 7: JP-A 2015-63472
Patent Document 8: JP 5573098
Patent Document 9: JP 6461919
Patent Document 10: JP 5544078
Patent Document 11: JP 5609569
Patent Document 12: JP-A 2024-26915

### SUMMARY OF THE INVENTION

In response to a recent demand for high resolution of a resist pattern, a conventional resist composition using an onium salt type photoacid generator cannot sufficiently suppress acid diffusion, and as a result, lithographic performance such as contrast, LWR, CDU, MEF, exposure latitude (EL), or depth of focus (DOF) is degraded. There is a problem that pattern collapse due to swelling occurs at the time of small-size pattern formation.

An object of the invention is to provide an onium salt and a chemically amplified resist composition comprising the onium salt as a photoacid generator, the resist composition having a high solvent solubility and a high sensitivity and being improved in lithography properties such as LWR, CDU, MEF, EL, and DOF with high contrast when processed by photolithography using high-energy radiation such as KrF or ArF excimer laser, EB, or EUV; and a pattern forming process using the chemically amplified resist composition.

The inventor has found that an onium salt containing a fluoroalkanesulfonic acid anion having an aromatic ring in which a hydroxy group protected with an acid labile group and a pentafluorosulfanyl group (—SF₅ group) are bonded to adjacent carbon atoms has a high solvent solubility, a chemically amplified resist composition using the onium salt as a photoacid generator has a high sensitivity, high contrast, and improved lithography properties such as LWR, CDU, MEF, EL, and DOF, and is extremely effective in suppressing pattern collapse during formation of small-size patterns.

That is, the invention provides an onium salt, a chemically amplified resist composition, and a pattern forming process.

1. An onium salt having the formula (1):

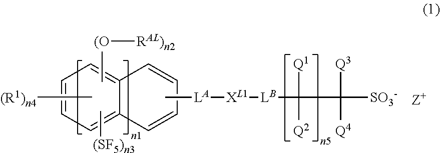

wherein n1 is 0 or 1, n2 is 1, 2, or 3, n3 is 1 or 2, n4 is 0, 1, or 2, n5 is 0, 1, 2, 3, or 4, provided that 2≤n2+n3+n4≤5 when n1 is 0, 2≤n2+n3+n4≤7 when n1 is 1,
R^{AL} is an acid labile group, at least one-SFs group and at least one —O—R^{AL} are bonded to adjacent carbon atoms,
R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n4 is 2, two R¹ may be identical or different and two R¹ may bond together to form a ring with the carbon atom to which they are attached,
L^{A} and L^{B} are each independently a single bond, an ether bond, an ester bond, a sulfonate ester bond, an amide bond, a sulfonamide bond, a carbonate bond, or a carbamate bond,
X^{L1} is a single bond or a C₁-C₄₀ hydrocarbylene group which may contain a heteroatom,
Q¹ and Q² are each independently a hydrogen atom, a fluorine atom, or a C₁-C₆ fluorinated saturated hydrocarbyl group,
Q³ and Q⁴ are each independently a fluorine atom or a C₁-C₆ fluorinated saturated hydrocarbyl group, and
Z⁺ is an onium cation.

2. The onium salt of 1 having the formula (1A):

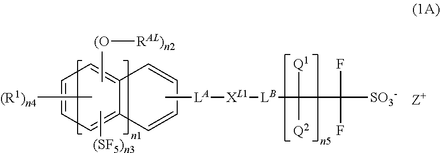

wherein n1 to n5, R^{AL}, R¹, L^{A}, L^{B}, X^{L1}, Q¹, Q², and Z⁺ are as defined above.

3. The onium salt of 2 having the formula (1B):

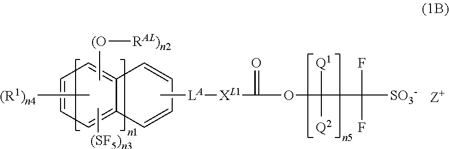

wherein n1 to n5, R^{AL}, R¹, L^{A}, X^{L1}, Q¹, Q², and Z⁺ are as defined above.

4. The onium salt of any one of 1 to 3 wherein a structure of an acid labile group formed together with an adjacent oxygen atom has the formula (AL-1) or (AL-2):

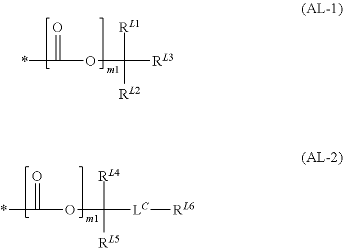

wherein n6 is 0 or 1, n7 is 0 or 1,
R^{L1}, R^{L2}, and R^{L3} are each independently a C₁-C₁₂ hydrocarbyl group, a part of —CH₂—of the hydrocarbyl group may be substituted with —O— or —S—, when the hydrocarbyl group contains an aromatic ring, some or all of the hydrogen atoms of the aromatic ring may be substituted with a halogen atom, a cyano group, a nitro group, a C₁-C₄ alkyl group which may contain a halogen atom, or a C₁-C₄ alkoxy group which may contain a halogen atom, R^{L1} and R¹² may bond together to form a ring with the carbon atom to which they are attached, a part of —CH₂— of the ring may be substituted with —O— or —S—,
R^{L4} and R^{L5} are each independently a hydrogen atom or a C₁-C₁₀ hydrocarbyl group, R^{L6} is a C₁-C₂₀ hydrocarbyl group, a part of —CH₂— of the hydrocarbyl group may be substituted with —O— or —S—, R^{L5} and R^{L6} may bond together to form a C₃-C₂₀ heterocyclic group with the carbon atom to which they are attached and L^{C}, a part of —CH₂— of the heterocyclic group may be substituted with —O— or —S—,
L^{C} is-O— or —S—, and
* designates a point of attachment to adjacent —O—.

5. The onium salt of any one of 1 to 4 wherein Z⁺ is a sulfonium cation having the formula (Z-1) or an iodonium cation having the formula (Z-2):

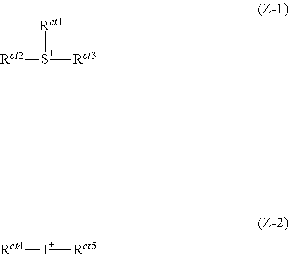

wherein R^{ct1} to R^{ct5} are each independently a halogen atom or a C₁-C₃₀ hydrocarbyl group which may contain a heteroatom, and R^{ct1} and R^{ct2} may bond together to form a ring with the sulfur atom to which they are attached.

6. The onium salt of any one of 1 to 4 wherein Z⁺ is a sulfonium cation having the formula (Z-3):

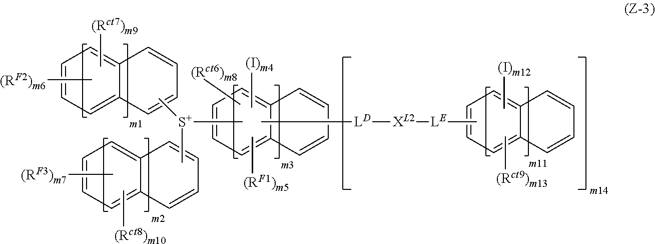

wherein m1 is 0 or 1, m2 is 0 or 1, m3 is 0 or 1, m4 is 0, 1, 2, 3, or 4, m5 is 0, 1, 2, 3, or 4, m6 is 0, 1, 2, 3, 4, 5, or 6, m7 is 0, 1, 2, 3, 4, 5, or 6, m8 is 0, 1, or 2, m9 is 0, 1, or 2, m10 is 0, 1, or 2, m11 is 0 or 1, m12 is 0, 1, 2, 3, or 4, m13 is 0, 1, or 2, m14 is 0, 1, or 2, provided that 0≤m6+m9≤4 when m1 is 0, 0≤m6+m9≤6 when m1 is 1, 0≤m7+m10≤4 when m2 is 0, 0≤m7+m10≤6 when m2 is 1, 1≤m4+m5+m8+m14≤4 when m3 is 0, 1≤m4+m5+m8+m14≤6 when m3 is 1, 0≤m12+m13≤4 when m11 is 0,0≤m12+m13≤6 when m11 is 1, m4+m12≥1,
R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, R^{F1} may be identical or different when m5 is 2 or more, R^{F2} may be identical or different when m6 is 2 or more, R^{F3} may be identical or different when m7 is 2 or more,
R^{ct6} to R^{ct9} are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m8 is 2, two R^{ct6} may be identical or different and two R^{ct6} may bond together to form a ring with the carbon atom to which they are attached, when m9 is 2, two R^{ct7} may be identical or different and two R^{ct7} may bond together to form a ring with the carbon atom to which they are attached, when m10 is 2, two R^{ct8} may be identical or different and two R^{ct8} may bond together to form a ring with the carbon atom to which they are attached, when m13 is 2, two R^{ct9} may be identical or different and two R^{ct9} may bond together to form a ring with the carbon atom to which they are attached,
aromatic rings which are directly bonded to S⁺ in the sulfonium cation may bond together to form a ring with S⁺,
L^{D} and L^{E} are each independently a single bond, an ether bond, an ester bond, an amide bond, a sulfonate ester bond, a sulfonamide bond, a carbonate bond, or a carbamate bond, and
X^{L2} is a single bond or a C₁-C₄₀ hydrocarbylene group which may contain a heteroatom.

7. A photoacid generator comprising the onium salt of any one of 1 to 6.

8. A chemically amplified resist composition comprising the photoacid generator of 7.

9. The chemically amplified resist composition of 8 further comprising a base polymer comprising at least one selected from a repeat unit having the formula (a1), a repeat unit having the formula (a2), and a repeat unit having the formula (a3):

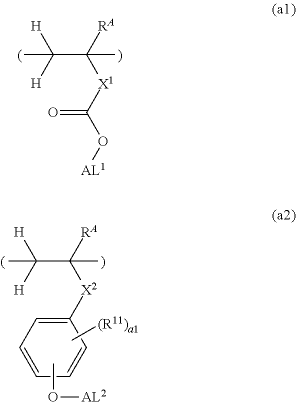

wherein R^{A} is each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, the phenylene group or the naphthylene group may be substituted with a hydroxy group, a nitro group, a cyano group, an optionally fluorinated C₁-C₁₀ saturated hydrocarbyl group, an optionally fluorinated C₁-C₁₀ saturated hydrocarbyloxy group, or a halogen atom, X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring,
X² is a single bond, *—C(═O)—O—, or *—C(═O)—NH—,
* designates a point of attachment to the carbon atom in the backbone,
R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, R¹¹ may be identical or different when a1 is 2, 3, or 4,
A^{L1} and A^{L2} are each independently an acid labile group, and
a1 is 0, 1, 2, 3, or 4,

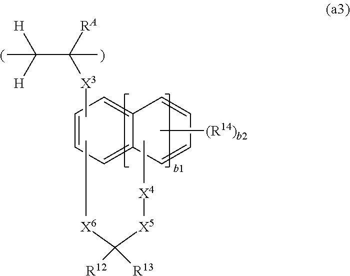

wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to the carbon atom in the backbone,
X⁴ is a single bond, a C₁-C₄ aliphatic hydrocarbylene group, a carbonyl group, a sulfonyl group, or a group obtained by combining the foregoing,
X⁵ and X⁶ are each independently an oxygen atom or a sulfur atom, provided that X⁴ and X⁶ are bonded to adjacent carbon atoms of the aromatic ring,
R¹² and R¹³ are each independently a hydrogen atom or a C₁-C₂₀ hydrocarbyl group which may contain a heteroatom, R¹² and R¹³ may bond together to form a ring with the carbon atom to which they are attached,
R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently a hydrogen atom or a C₁-C₆ hydrocarbyl group, and when b2 is 2 or more, R¹⁴ may be identical or different, and a plurality of R¹⁴ may bond together to form a ring with the carbon atom of the aromatic ring to which they are attached.

10. The chemically amplified resist composition of 9 wherein the base polymer comprises at least one selected from a repeat unit having the formula (b1) and a repeat unit having the formula (b2):

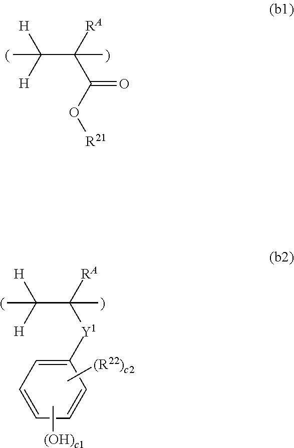

wherein R^{A} is each independently a hydrogen atom, a fluorine atom, a methyl group, or a trifluoromethyl group,
Y¹ is a single bond or *—C(═O)—O—, * designates a point of attachment to the carbon atom in the backbone,
R²¹ is a hydrogen atom or a C₁-C₂₀ group having at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—),
R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, R²² may be identical or different when c2 is 2, 3, or 4, c1 is 1, 2, 3, or 4, and c2 is 0, 1, 2, 3, or 4, provided that 1≤c1+c2≤5.

11. The chemically amplified resist composition of 9 or 10 wherein the base polymer comprises at least one selected from a repeat unit having the formula (c1), a repeat unit having the formula (c2), a repeat unit having the formula (c3), a repeat unit having the formula (c4), and a repeat unit having the formula (c5):

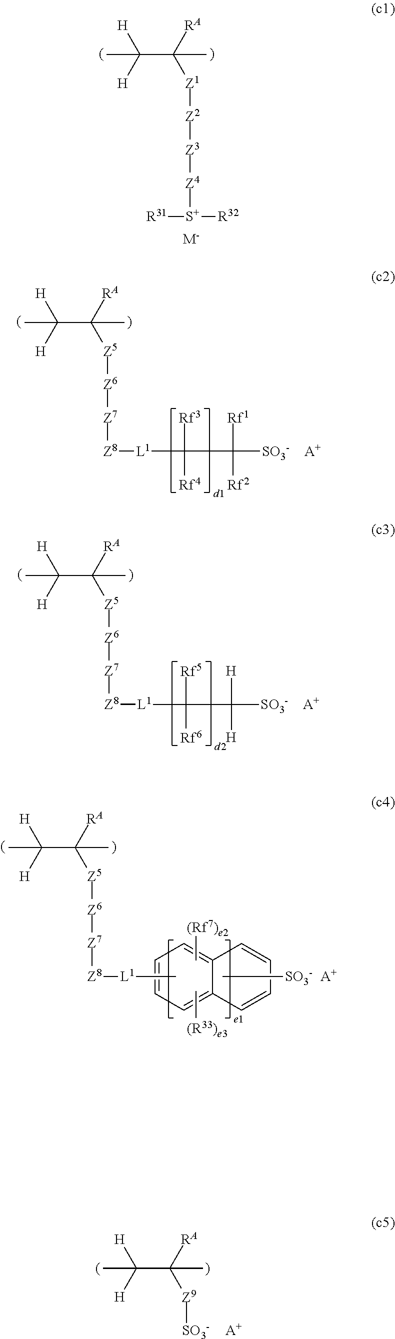

wherein d1 and d2 are each independently 0, 1, 2, or 3, e1 is 0 or 1, e2 is 0, 1, 2, 3, or 4, e3 is 0, 1, 2, 3, or 4, provided that 0≤e2+e3≤4 when e1 is 0, 0≤e2+e3≤6 when e1 is 1,
R^{A} is each independently a hydrogen atom, a fluorine atom, a methyl group, or a trifluoromethyl group,
Z¹ is a single bond or a phenylene group which may have a substituent, Z² is a single bond, **—C(═O)—O—Z²¹—, **—C(═O)—NH—Z²¹—, or **—O—Z²¹—, Z²¹ is a C₁-C₆ aliphatic hydrocarbylene group, a phenylene group, or a divalent group obtained by combining the foregoing, and may contain a halogen atom, a carbonyl group, an ester bond, an ether bond, or a hydroxy group,
Z³ is a single bond, an ether bond, an ester bond, an amide bond, a sulfonate ester bond, a sulfonamide bond, a carbonate bond, or a carbamate bond,
Z⁴ is a single bond, or a C₁-C₆ aliphatic hydrocarbylene group, a phenylene group, or a divalent group obtained by combining the foregoing, and may contain a halogen atom, a carbonyl group, an ester bond, an ether bond, or a hydroxy group,
Z⁵ is each independently a single bond, a phenylene group which may have a substituent, a naphthylene group, or *—C(═O)—O—Z⁵¹—, Z⁵¹ is a C₁-C₁₀ aliphatic hydrocarbylene group, a phenylene group, or a naphthylene group, the aliphatic hydrocarbylene group may contain a halogen atom, a hydroxy group, an ether bond, an ester bond, or a lactone ring,
Z⁶ is a single bond, an ether bond, an ester bond, an amide bond, a sulfonate ester bond, a sulfonamide bond, a carbonate bond, or a carbamate bond,
Z⁷ is each independently a single bond, ***—Z⁷¹—C(═O)—O—, ***—C(—O)—NH—Z⁷¹—, or ***—O—Z⁷¹—, Z⁷¹ is a C₁-C₂₀ hydrocarbylene group which may contain a heteroatom,
Z⁸ is each independently a single bond, ****—Z⁸¹—C(—O)—O—, ****—C(═O)—NH—Z⁸¹—, or ****—O—Z⁸¹—, Z⁸¹ is a C₁-C₂₀ hydrocarbylene group which may contain a heteroatom,
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—N(H)—Z⁹¹—, or *—O—Z⁹¹—, Z⁹¹ is a phenylene group substituted with a C₁-C₆ aliphatic hydrocarbylene group, a phenylene group, a fluorinated phenylene group, or a trifluoromethyl group, and may contain a carbonyl group, an ester bond, an ether bond, or a hydroxy group,
* designates a point of attachment to the carbon atom in the backbone, ** designates a point of attachment to Z¹, *** designates a point of attachment to Z⁶, **** designates a point of attachment to Z⁷,
L¹ is a single bond, an ether bond, an ester bond, a carbonyl group, a sulfonate ester bond, a sulfonamide bond, a carbonate bond, or a carbamate bond,
R^{f1} and R^{f2} are each independently a fluorine atom or a C₁-C₆ fluorinated saturated hydrocarbyl group,
R^{f3} and R^{f4} are each independently a hydrogen atom, a fluorine atom, or a C₁-C₆ fluorinated saturated hydrocarbyl group,
R^{f5} and R^{f6} are each independently a hydrogen atom, a fluorine atom, or a C₁-C₆ fluorinated saturated hydrocarbyl group, provided that not all R^{f5} and R^{f6} are hydrogen atoms at the same time,
Rf⁷ is a fluorine atom, a C₁-C₆ fluorinated alkyl group, a C₁-C₆ fluorinated alkoxy group, or a C₁-C₆ fluorinated alkylthio group,
R³¹ and R³² are each independently a C₁-C₂₀ hydrocarbyl group which may contain a heteroatom, R³¹ and R³² may bond together to form a ring with the sulfur atom to which they are attached,
R³³ is a halogen atom other than a fluorine atom, or a C₁-C₂₀ hydrocarbyl group which may contain a heteroatom, when e3 is 2, 3, or 4, R³³ may be identical or different, a plurality of R³³ may bond together to form a ring with the carbon atom to which they are attached,
M⁻ is a non-nucleophilic counter ion, and
A⁺ is an onium cation.

12. The chemically amplified resist composition of any one of 8 to 11 further comprising an organic solvent.

13. The chemically amplified resist composition of any one of 8 to 12 further comprising a quencher.

14. The chemically amplified resist composition of any one of 8 to 13 further comprising a photoacid generator other than the photoacid generator of 7.

15. The chemically amplified resist composition of any one of 8 to 14 further comprising a surfactant.

16. A pattern forming process comprising the steps of forming a resist film on a substrate using the chemically amplified resist composition of any one of 8 to 15, exposing the resist film to high-energy radiation, and developing the exposed resist film in a developer.

17. The pattern forming process of 16 wherein the high-energy radiation is KrF excimer laser radiation, ArF excimer laser radiation, EB, or EUV of wavelength 3 to 15 nm.

### Advantageous Effects of the Invention

When the chemically amplified resist composition comprising the onium salt as a photoacid generator is processed by lithography, a resist pattern having a high contrast, high sensitivity, improved lithography properties such as LWR, CDU, MEF, EL, and DOF, and suppressed pattern collapse can be formed.

### DESCRIPTION OF THE PREFERRED EMBODIMENT

### Onium Salt

The invention provides an onium salt having the formula (1).

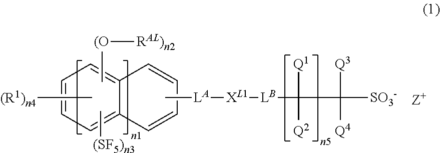

In formula (1), n1 is 0 or 1. The relevant structure is a benzene ring when n1 is 0, and a naphthalene ring when n1 is 1. From the viewpoint of solvent solubility, the benzene ring corresponding to n1=0 is preferred. n2 is 1, 2, or 3. From the viewpoint of reactant availability, n2 is preferably 1 or 2. n3 is 1 or 2. From the viewpoint of reactant availability, n3 is preferably 1. n4 is 0, 1, or 2. From the viewpoint of reactant availability, n4 is preferably 0 or 1. n5 is 0, 1, 2, 3, or 4, preferably 0, 1, 2, or 3, more preferably 1. Provided that 2≤n2+n3+n4≤5 when n1 is 0, and 2≤n2+n3+n4≤7 when n1 is 1.

In formula (1), R^{AL} is an acid labile group. The acid labile group is preferably a group having the formula (AL-1) or (AL-2).

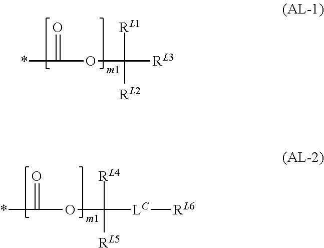

Herein * designates a point of attachment to —O—.

In formula (AL-1), n6 is 0 or 1. R^{L1}, R^{L2}, and R^{L3} are each independently a C₁-C₁₂ hydrocarbyl group, a part of —CH₂— of the hydrocarbyl group may be substituted with —O— or —S—, and when the hydrocarbyl group contains an aromatic ring, some or all of the hydrogen atoms of the aromatic ring may be substituted with a halogen atom, a cyano group, a nitro group, a C₁-C₄ alkyl group which may contain a halogen atom, or a C₁-C₄ alkoxy group which may contain a halogen atom. R^{L1} and R^{L2} may bond together to form a ring with the carbon atom to which they are attached, and a part of —CH₂— of the ring may be substituted with —O— or —S—.

In formula (AL-2), n7 is 0 or 1. R¹⁴ to R¹⁵ are each independently a hydrogen atom or a C₁-C₁₀ hydrocarbyl group. R^{L6} is a C₁-C₂₀ hydrocarbyl group, and a part of —CH₂—of the hydrocarbyl group may be substituted with —O— or —S—. R¹⁵ and R^{L6} may bond together to form a C₃-C₂₀ heterocyclic group with the carbon atom to which they are attached and L^{C}, and a part of —CH₂— of the heterocyclic group may be substituted with —O— or —S—. L^{C} is-O— or —S—.

Specific examples of the acid labile group having formula (AL-1) are shown below, but not limited thereto. * designates a point of attachment to adjacent —O—.

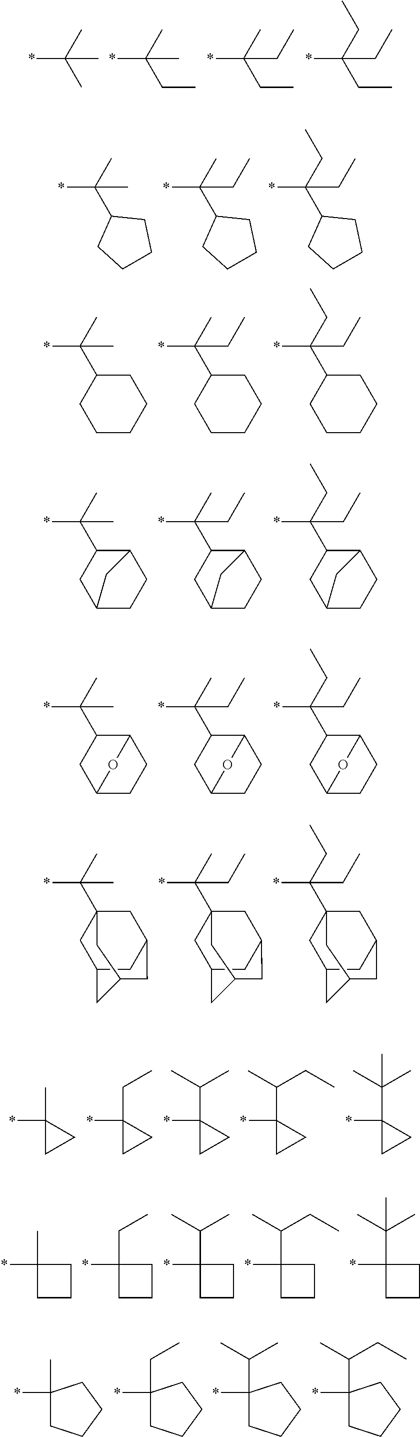

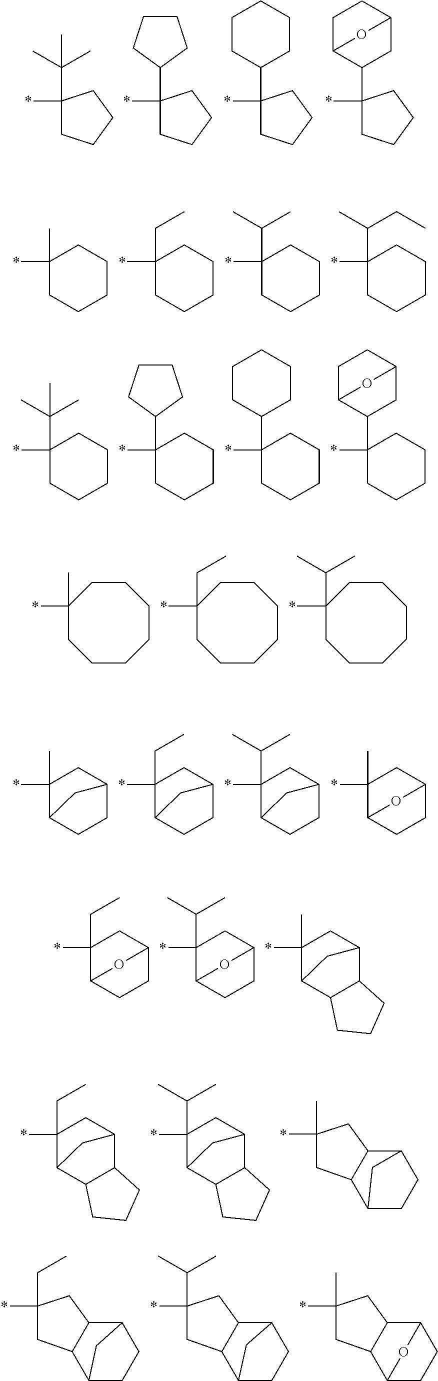

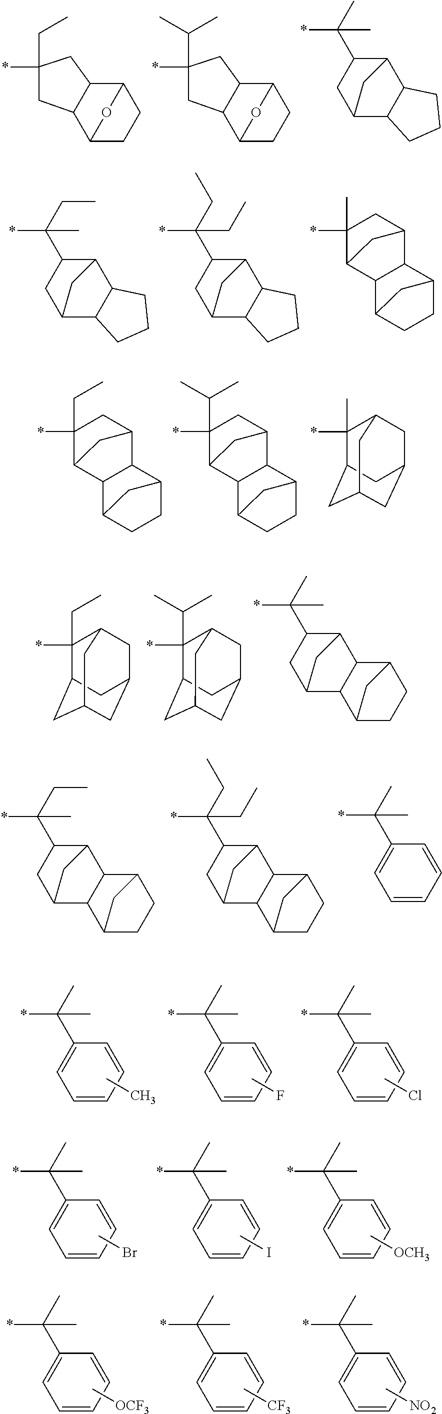

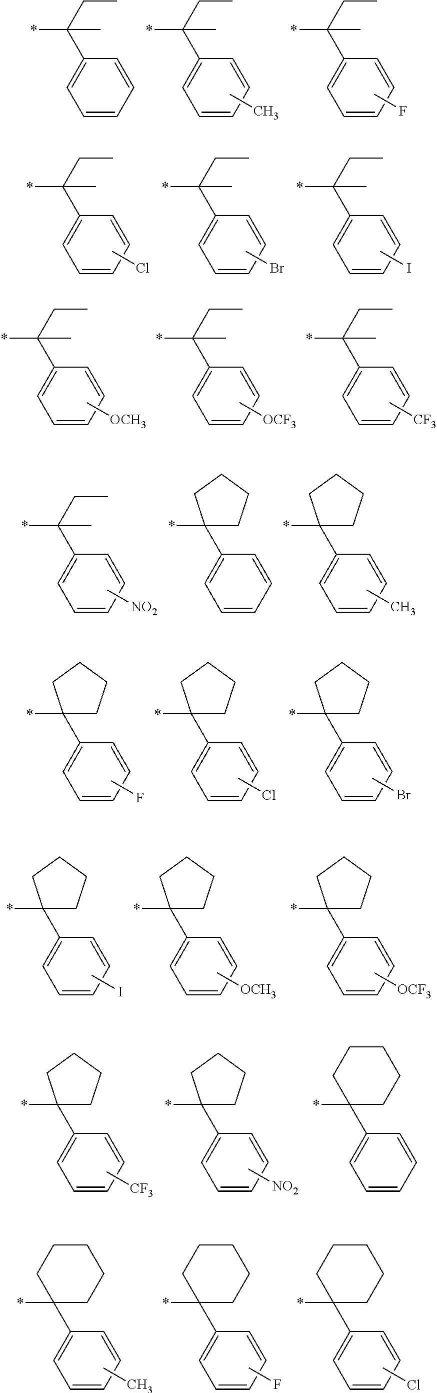

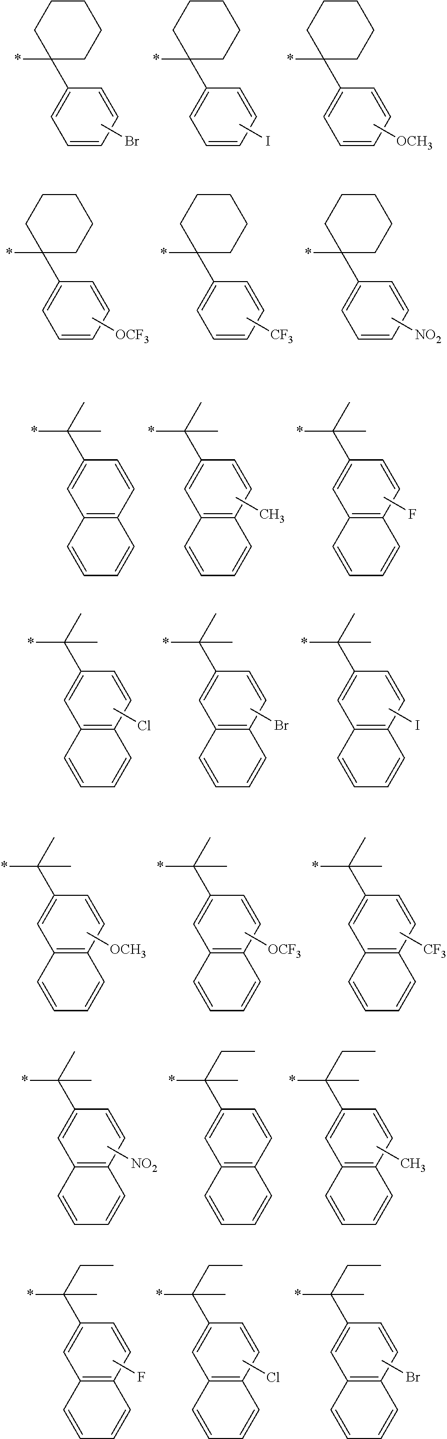

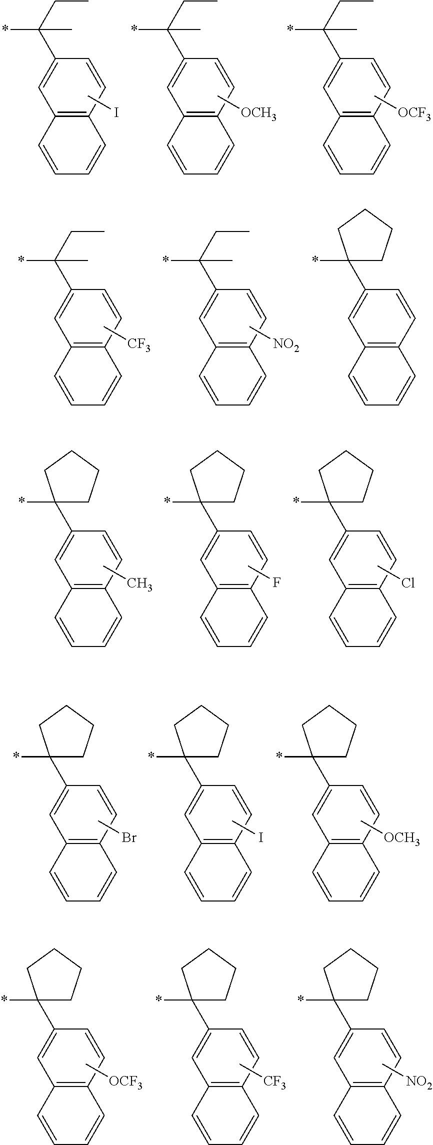

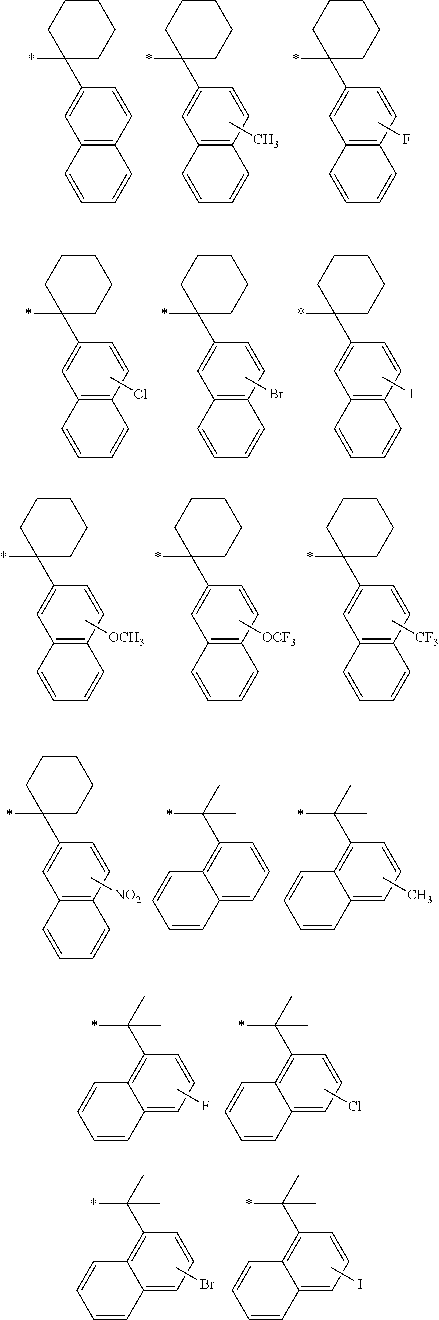

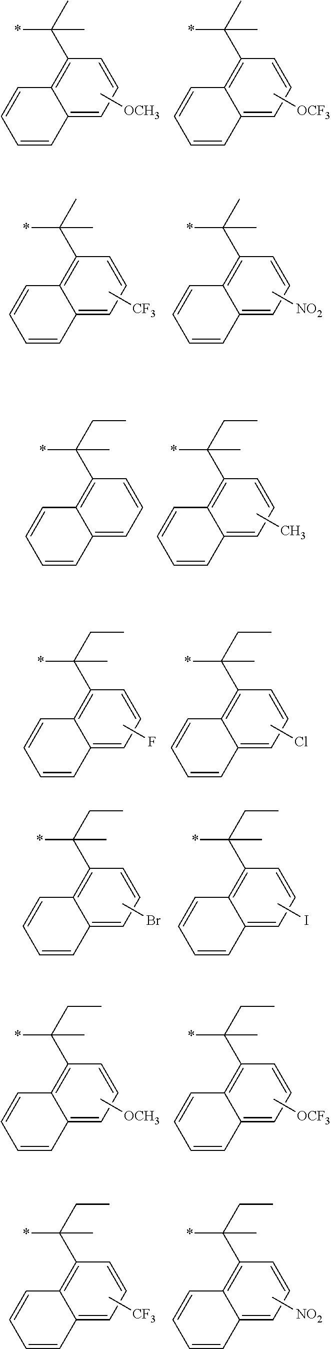

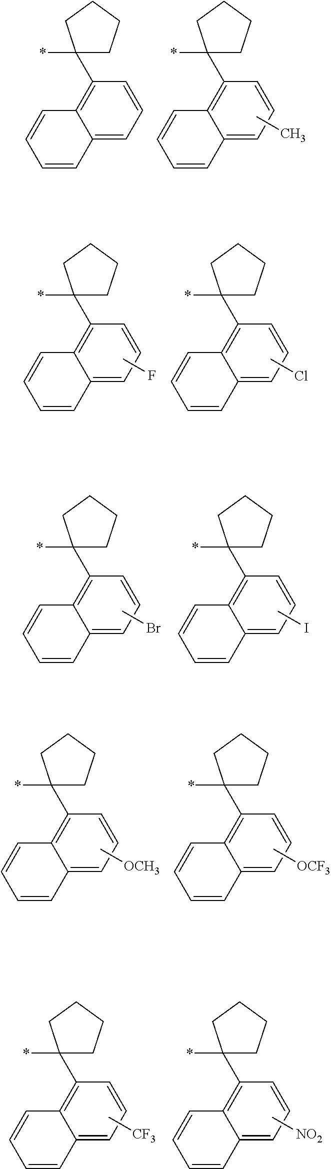

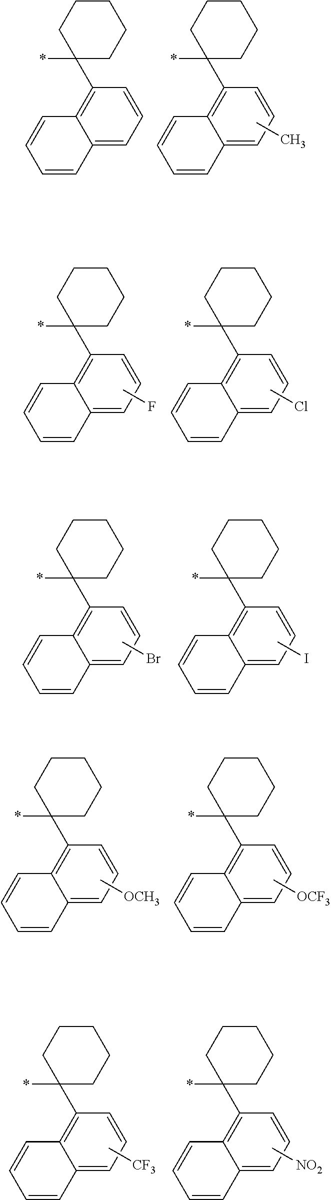

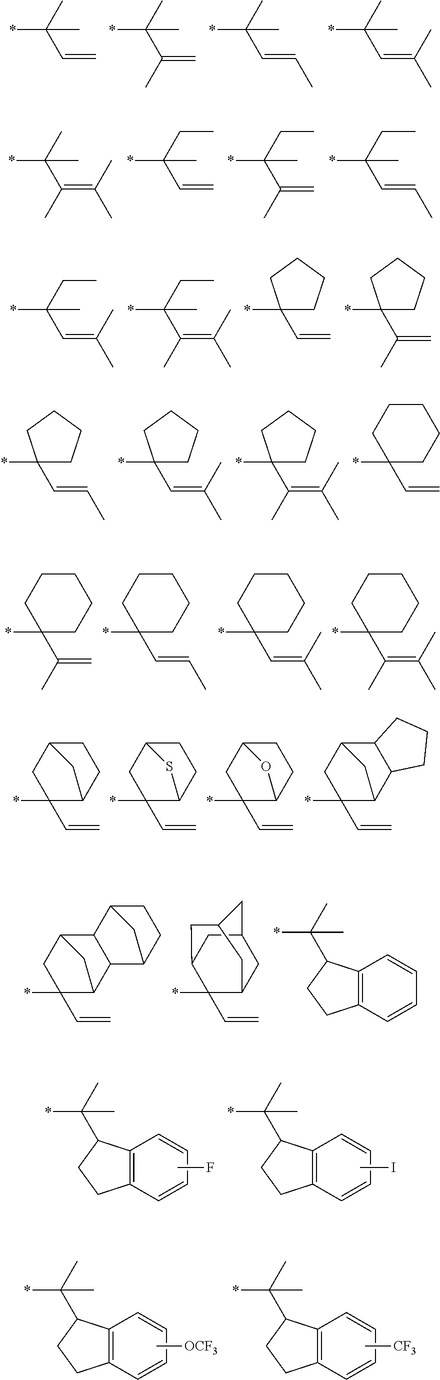

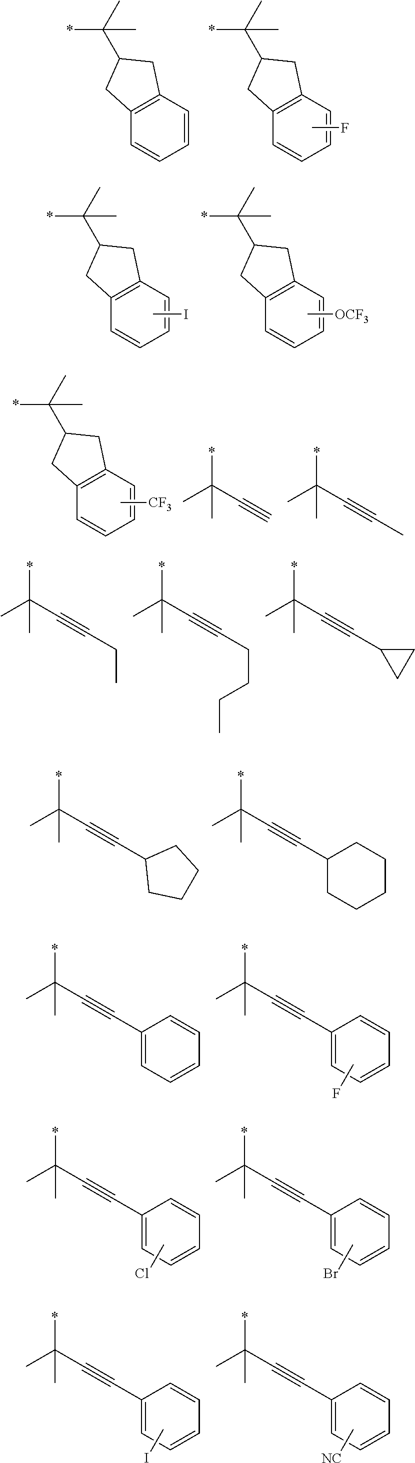

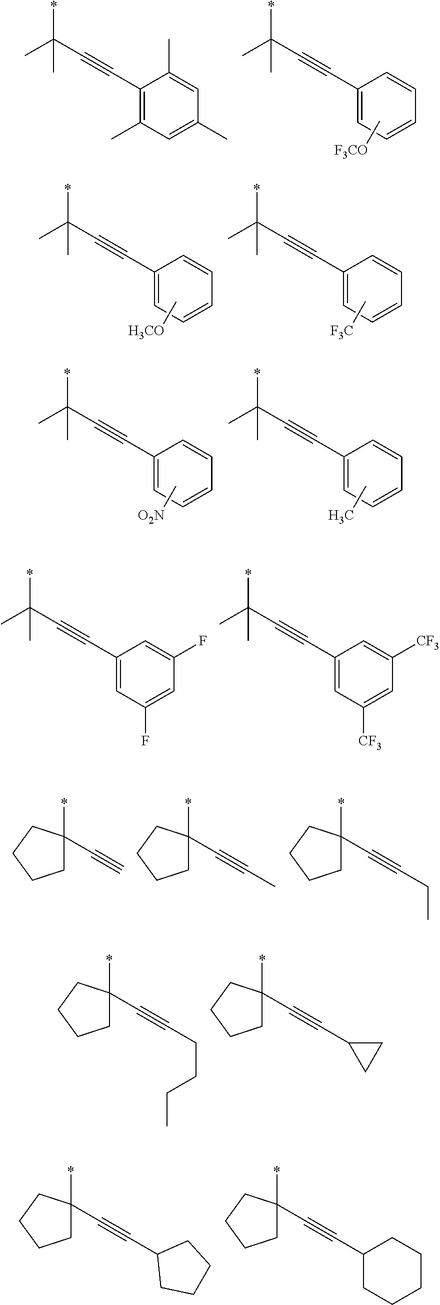

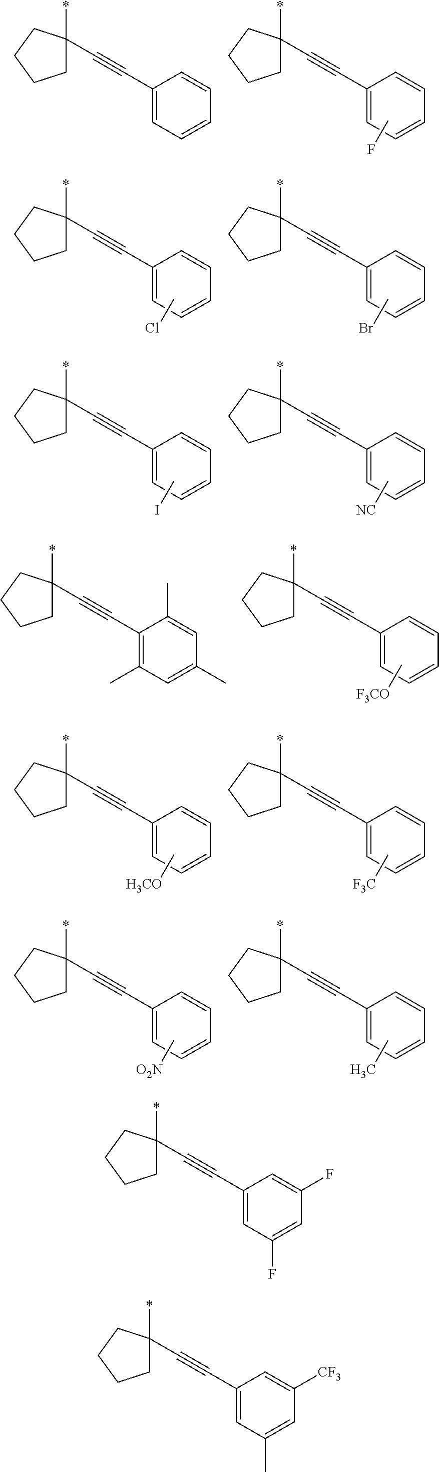

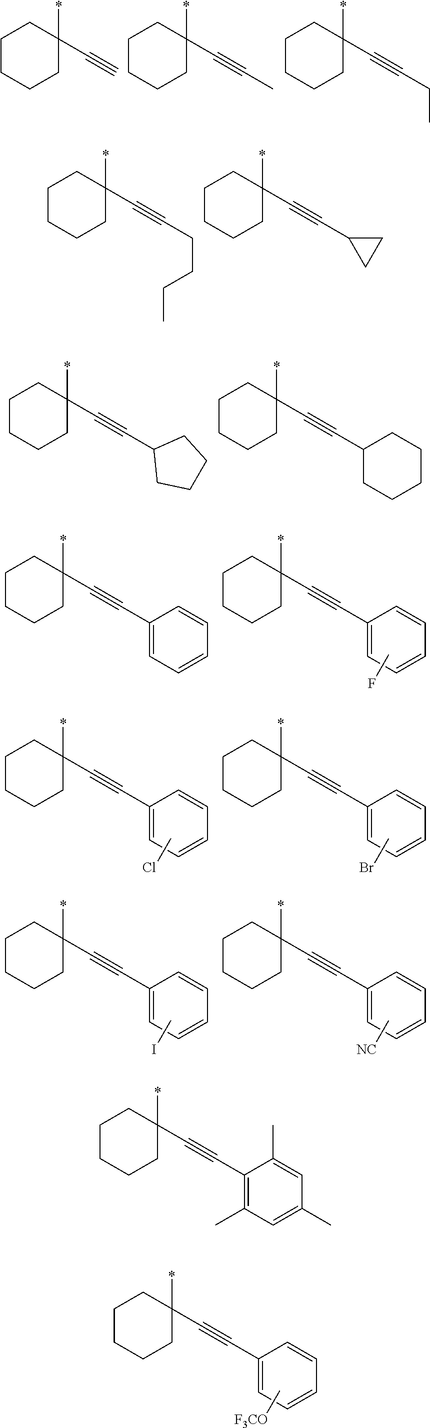

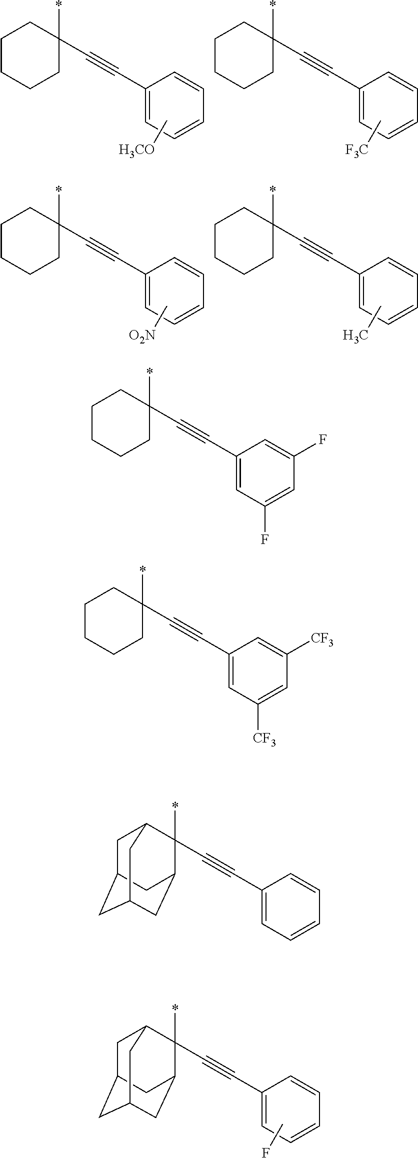

Specific examples of the acid labile group having formula (AL-2) are shown below, but not limited thereto. * designates a point of attachment to adjacent —O—.

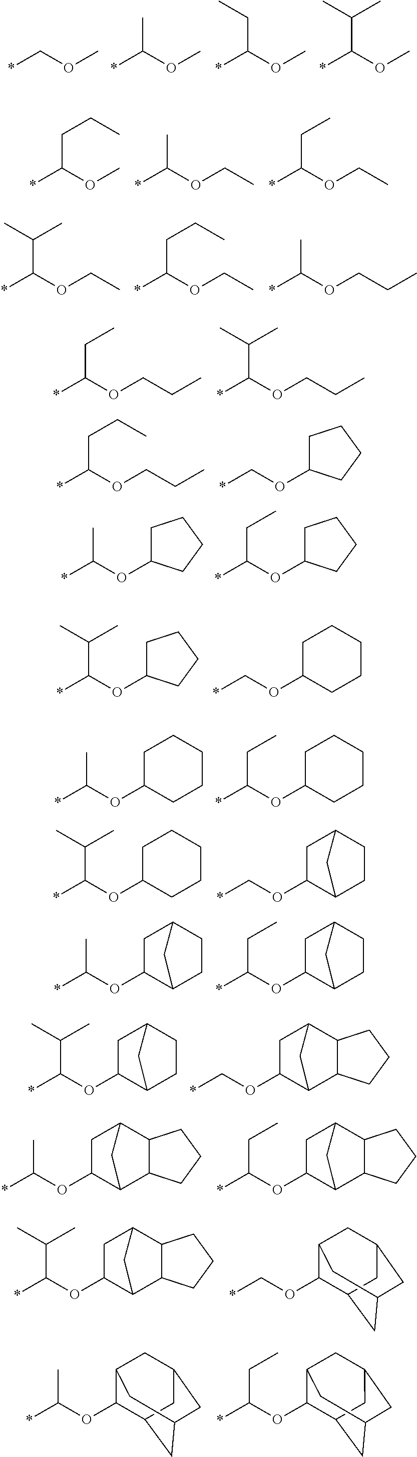

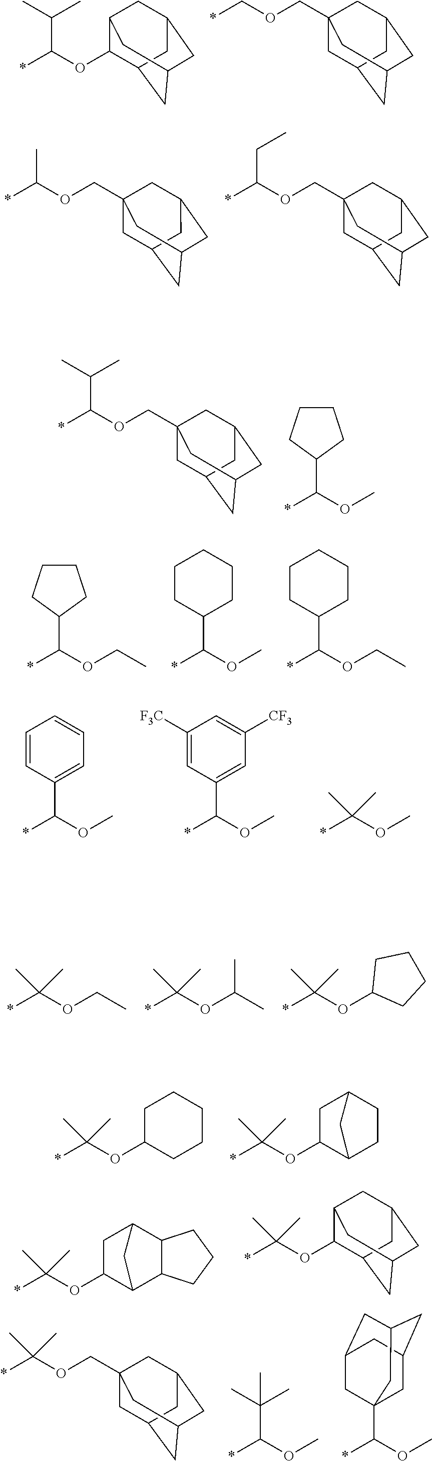

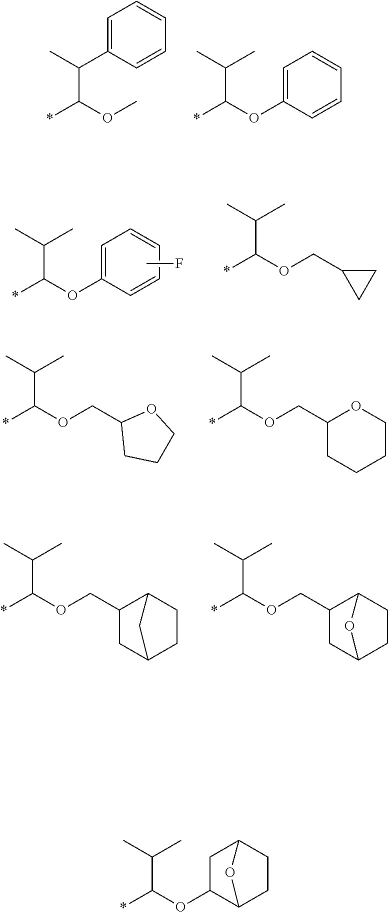

In formula (1), at least one-SFs group and at least one-O—R^{AL} bond together to adjacent carbon atoms. By being adjacent to each other, an acidity of the aromatic alcohol after the —R^{AL} is deprotected is improved, and a dissolution contrast is improved.

In formula (1), R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen atom include a fluorine atom, a chlorine atom, a bromine atom, and an iodine atom, and the halogen atom is preferably a fluorine atom or an iodine atom. The hydrocarbyl group may be saturated or unsaturated and straight, branched, or cyclic. Specific examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. Inter alia, aryl groups are preferred. In the hydrocarbyl group, some or all of the hydrogen atoms may be substituted by a moiety containing a heteroatom such as an oxygen atom, a sulfur atom, a nitrogen atom, or a halogen atom, and some constituent —CH₂— may be replaced by a moiety containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group. When n4 is 2, two R¹ may be identical or different. When n4 is 2, two R¹ may bond together to form a ring with the carbon atom to which they are attached. The ring is preferably a 5- to 8-membered ring.

In formula (1), L^{A} and L^{B} are each independently a single bond, an ether bond, an ester bond, a sulfonate ester bond, an amide bond, a sulfonamide bond, a carbonate bond, or a carbamate bond. Inter alia, a single bond, an ether bond, or an ester bond is preferred.

In formula (1), X^{L1} is a single bond or a C₁-C₄₀ hydrocarbylene group which may contain a heteroatom. The hydrocarbylene group may be straight, branched, or cyclic, and specific examples thereof include an alkanediyl group and a divalent saturated cyclic hydrocarbon group. Examples of the heteroatom include an oxygen atom, a nitrogen atom, and a sulfur atom.

Preferred examples of the C₁-C₄₀ hydrocarbylene group which may contain a heteroatom: X^{L1} are shown below. Herein * designates a point of attachment to L^{A} or L^{B}.

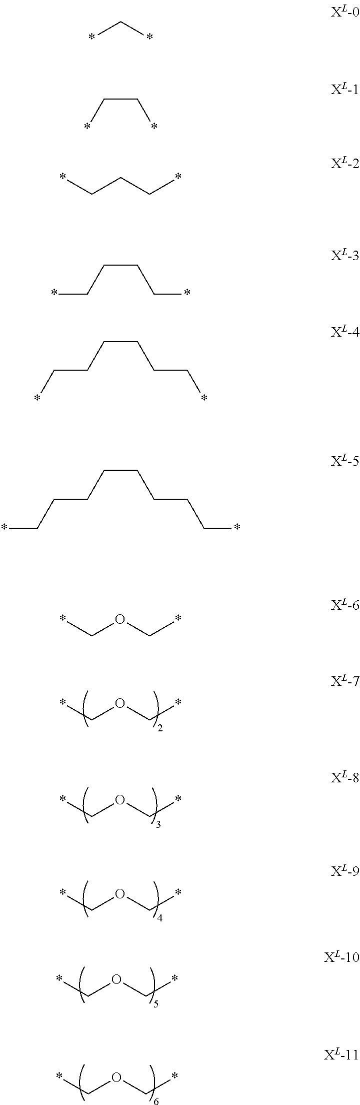

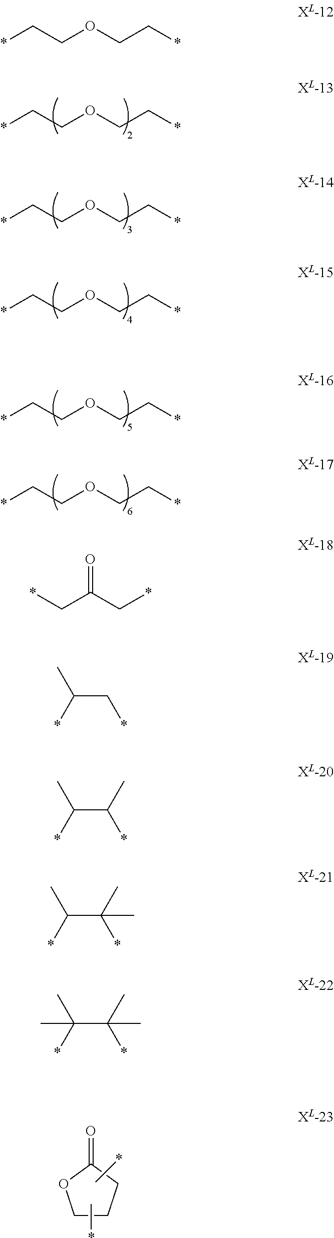

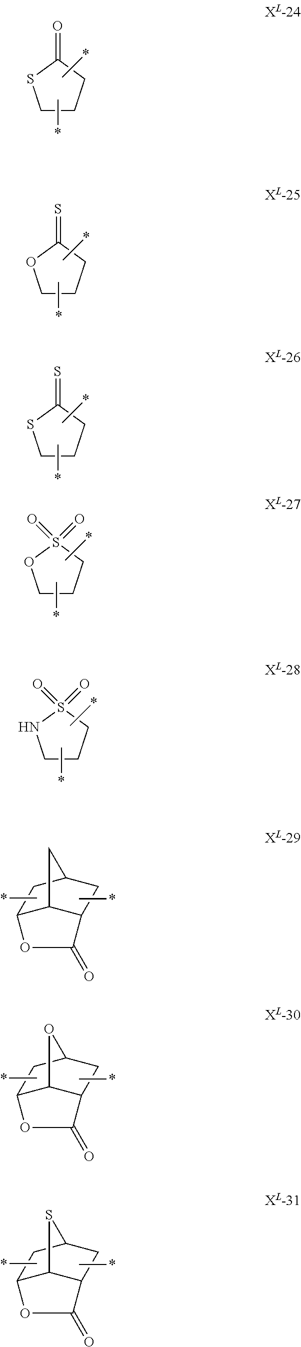

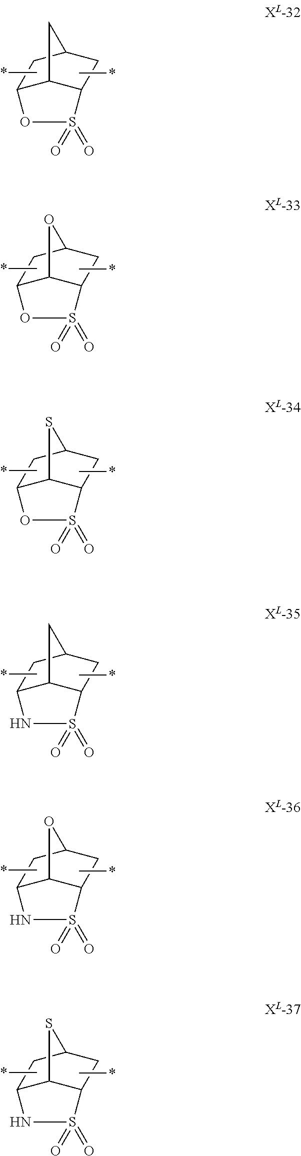

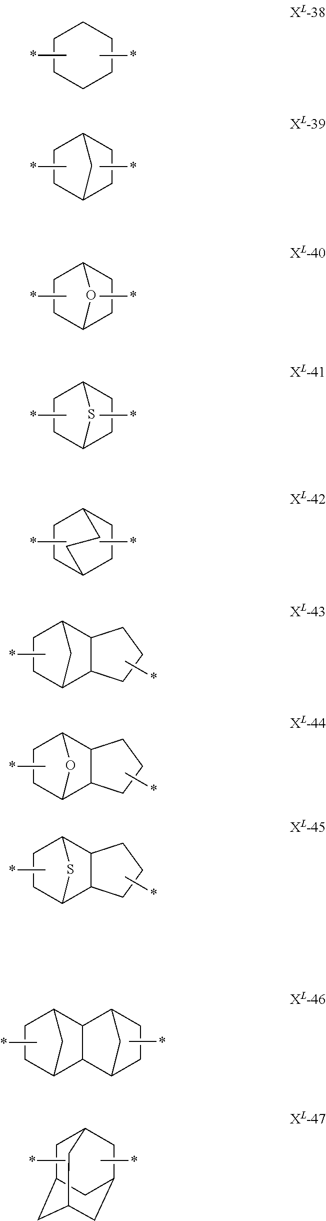

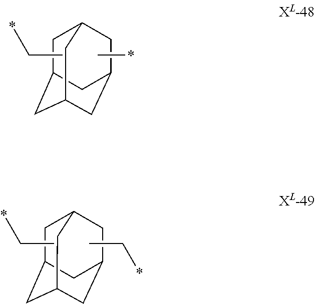

Inter alia, X^{L}-0 to X^{L}-3, X^{L}-29 to X^{L}-34, and X^{L}-47 to X^{L}-50 are preferred, and X^{L}-0 to X^{L}-2, X^{L}-29, and X^{L}-47 are more preferred.

In formula (1), Q¹ and Q² are each independently a hydrogen atom, a fluorine atom, or a C₁-C₆ fluorinated saturated hydrocarbyl group. The C₁-C₆ fluorinated saturated hydrocarbyl group is preferably a trifluoromethyl group.

In formula (1), Q³ and Q⁴ are each independently a fluorine atom, or a C₁-C₆ fluorinated saturated hydrocarbyl group. The C₁-C₆ fluorinated saturated hydrocarbyl group is preferably a trifluoromethyl group.

Preferred specific examples of the partial structure: —[C(Q¹)(Q²)]ₙ₅—C(Q³)(Q⁴)—SO₃⁻ in formula (1) are shown below, but not limited thereto. Herein * designates a point of attachment to L^{B}.

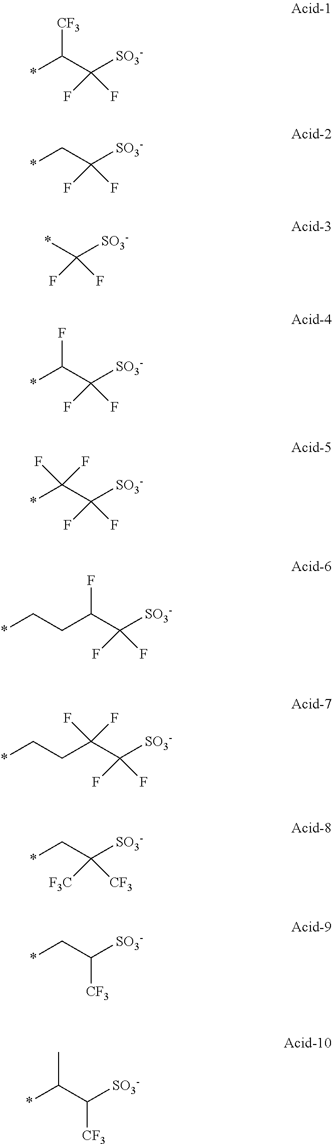

Inter alia, Acid-1 to Acid-7 are preferred, and Acid-1 to Acid-3, Acid-6, and Acid-7 are more preferred.

As the onium salt having formula (1), an onium salt having the formula (1A) is preferred:

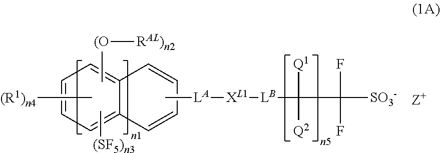

wherein n1 to n5, R^{AL}, R¹, L^{A}, L^{B}, X^{L1}, Q¹, Q², and Z⁺ are as defined above.

As the onium salt having formula (1A), an onium salt having the formula (1B) is preferred:

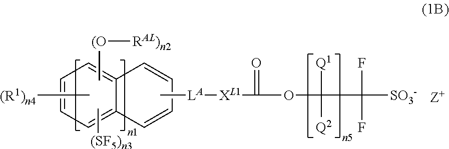

wherein n1 to n5, R^{AL}, R¹, L^{A}, X^{L1}, Q¹, Q², and Z⁺ are as defined above.

Examples of the anion of the onium salt having formula (1) are shown below, but not limited thereto. Herein Q¹ is as defined above, and Me is a methyl group. The substitution position of the substituent on the aromatic ring is not limited to this as long as at least one —O—R^{AL} and at least one-SFs are disposed adjacent to each other.

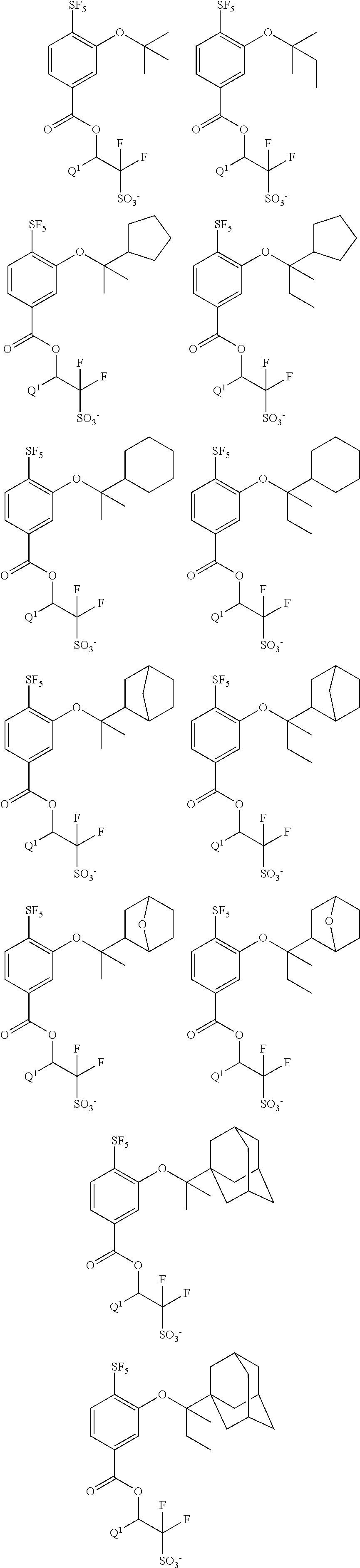

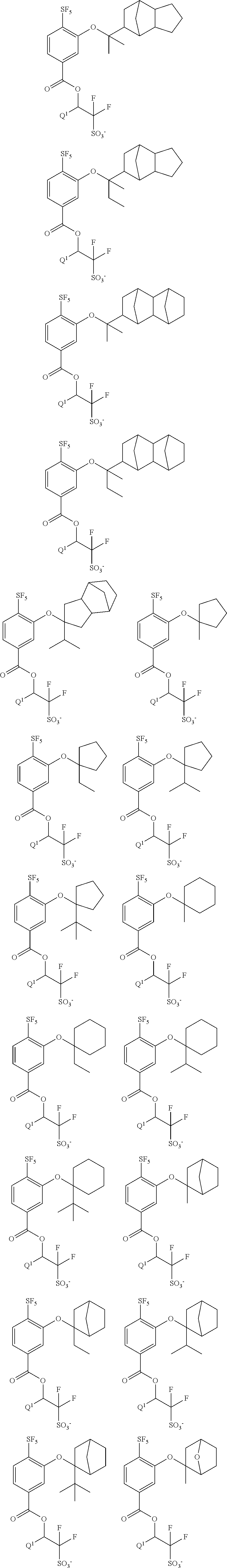

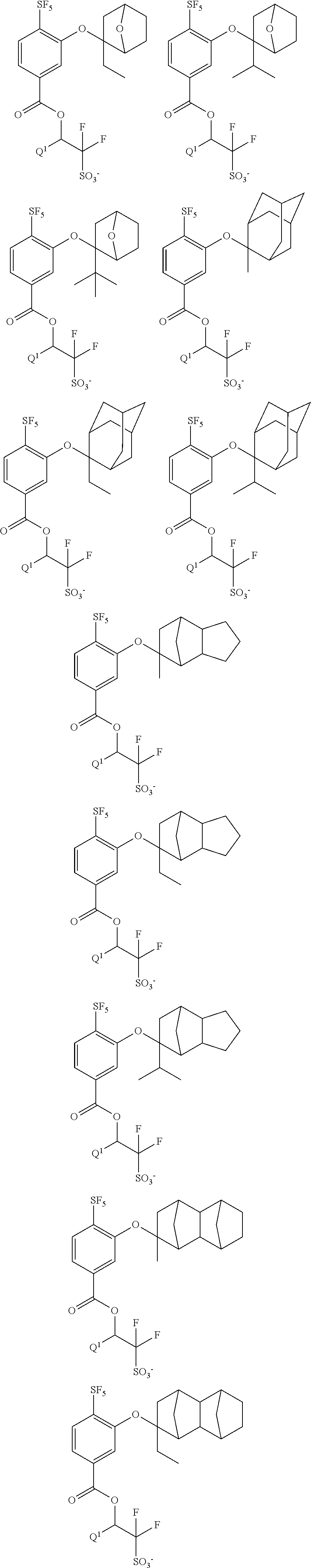

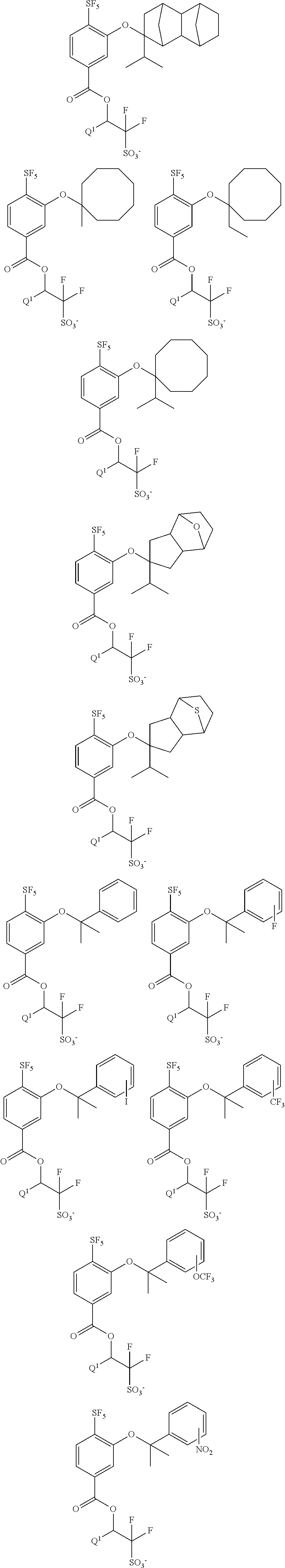

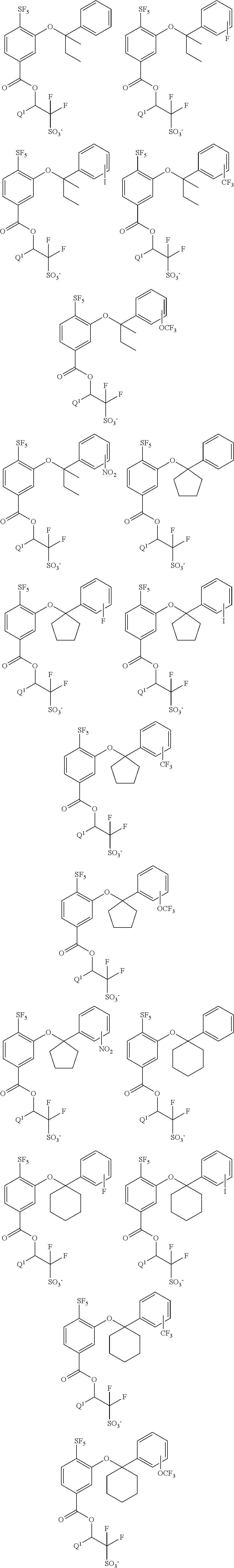

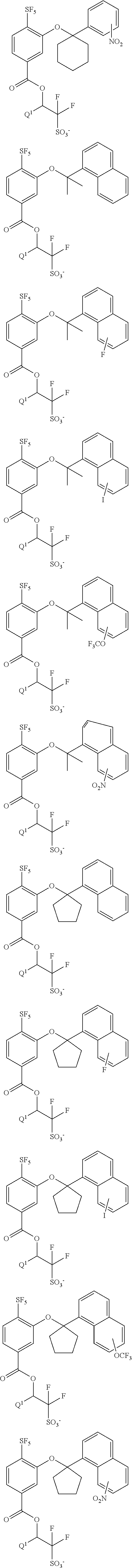

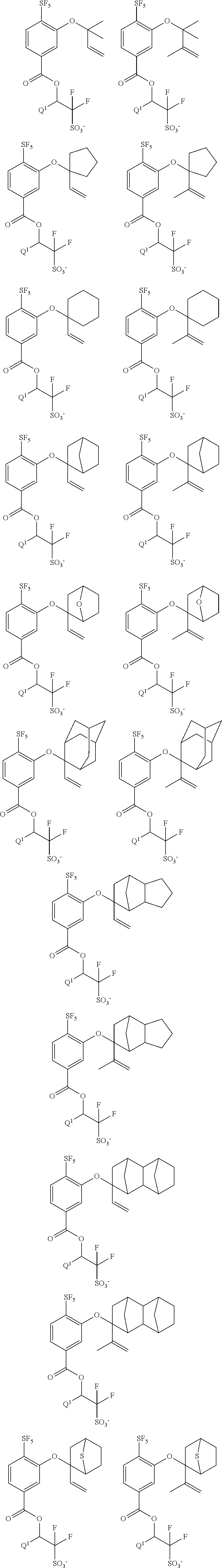

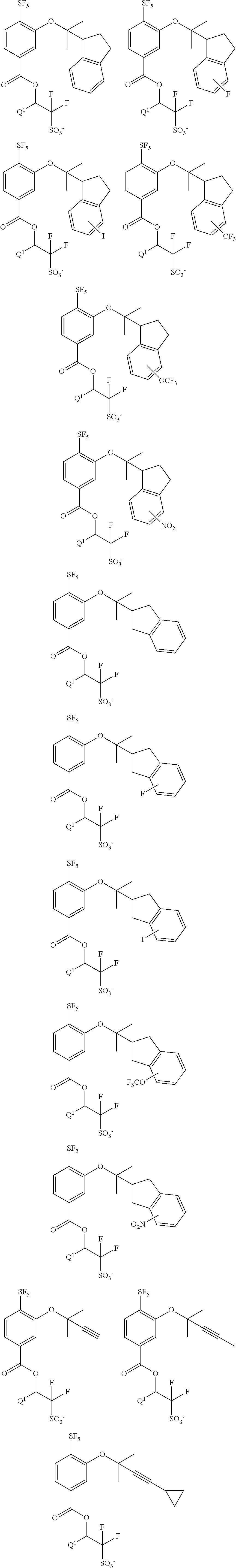

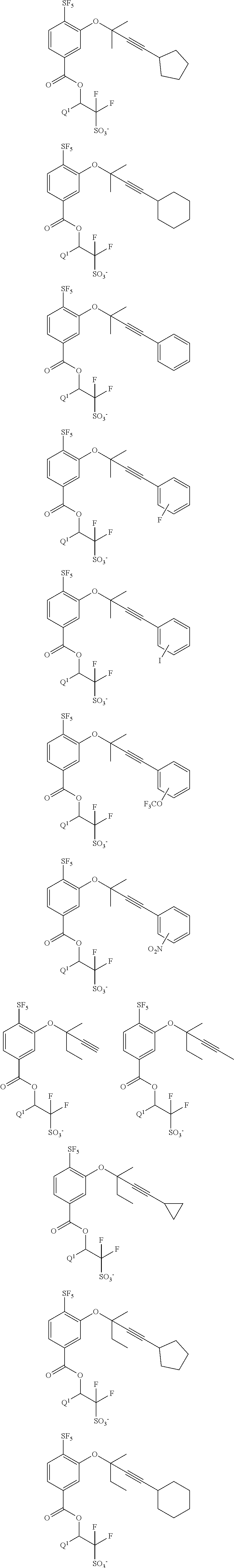

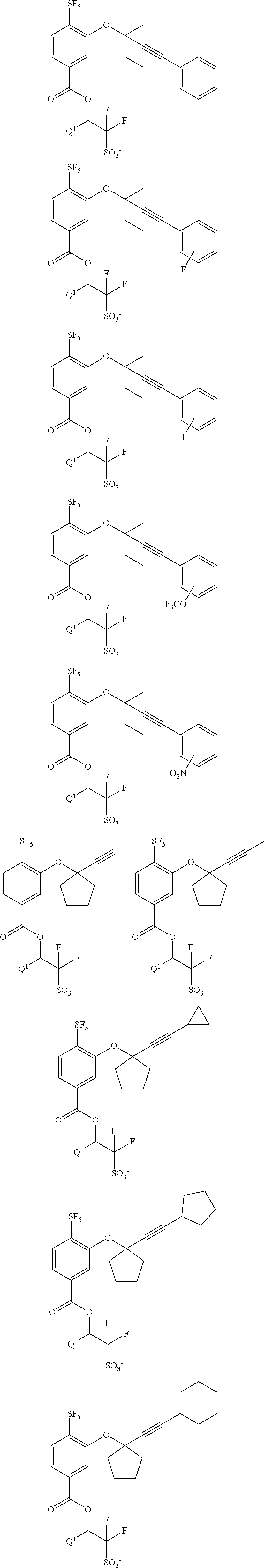

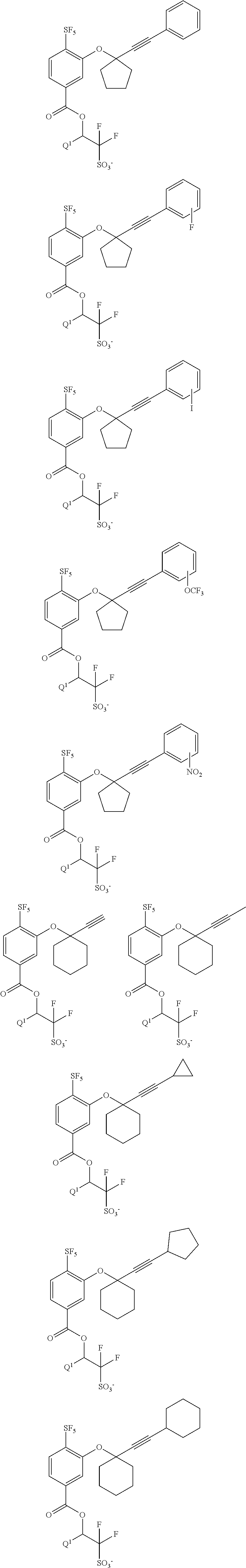

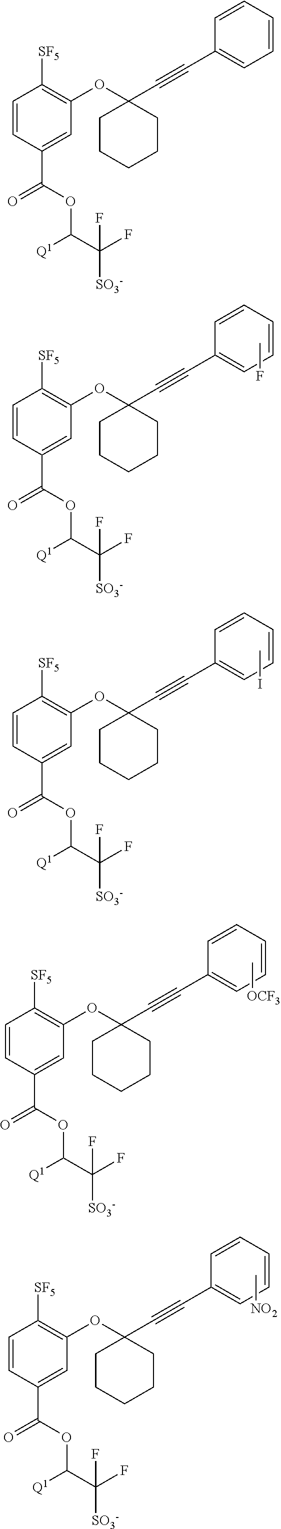

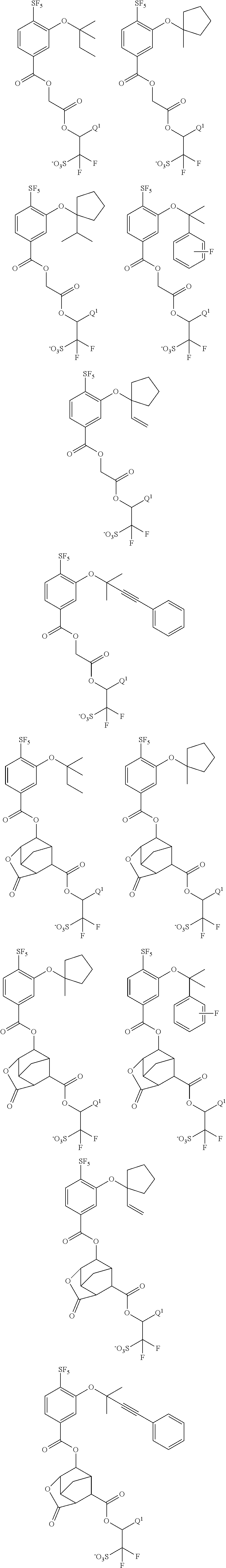

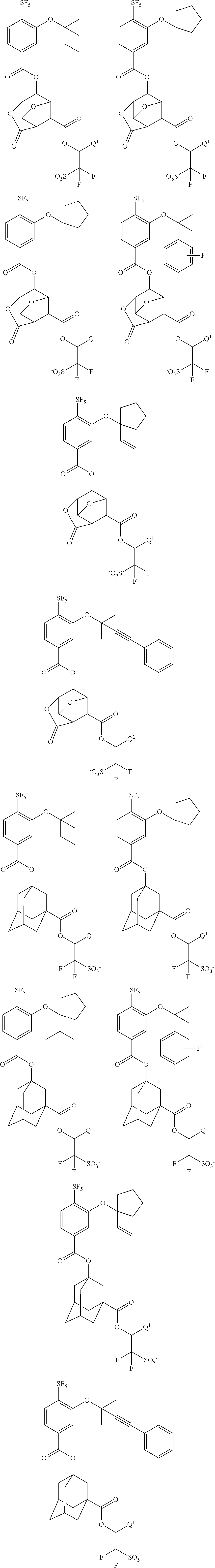

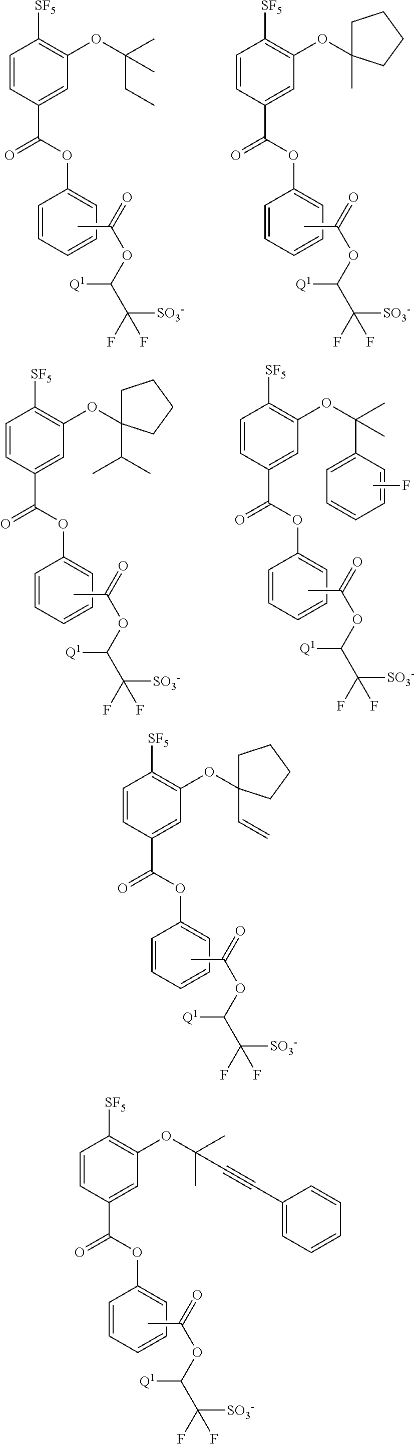

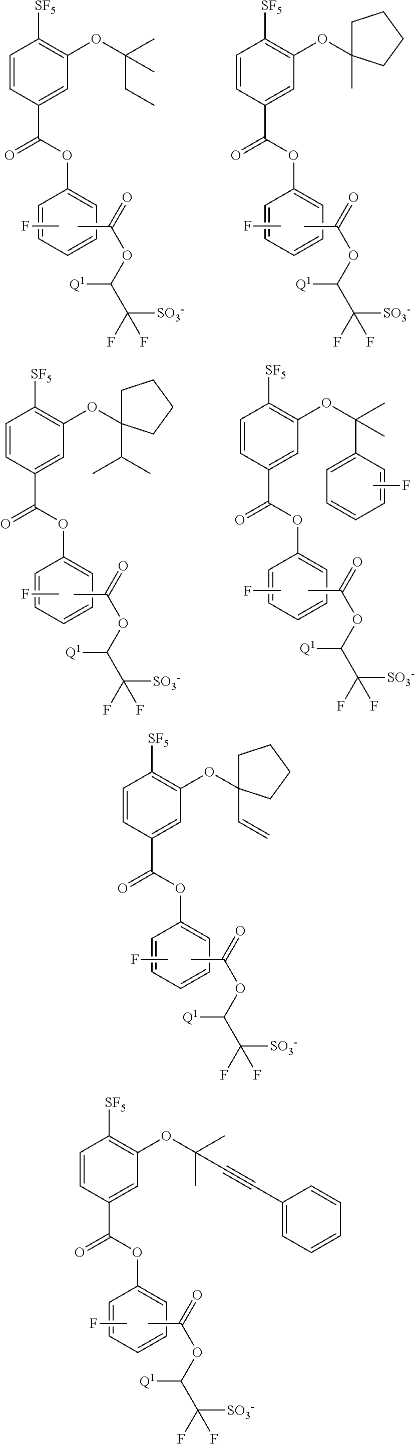

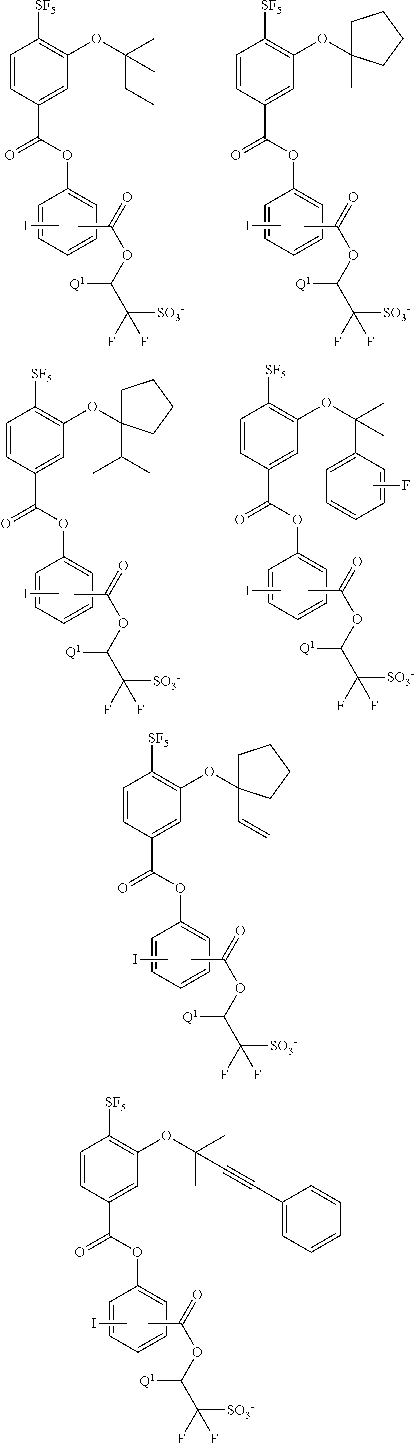

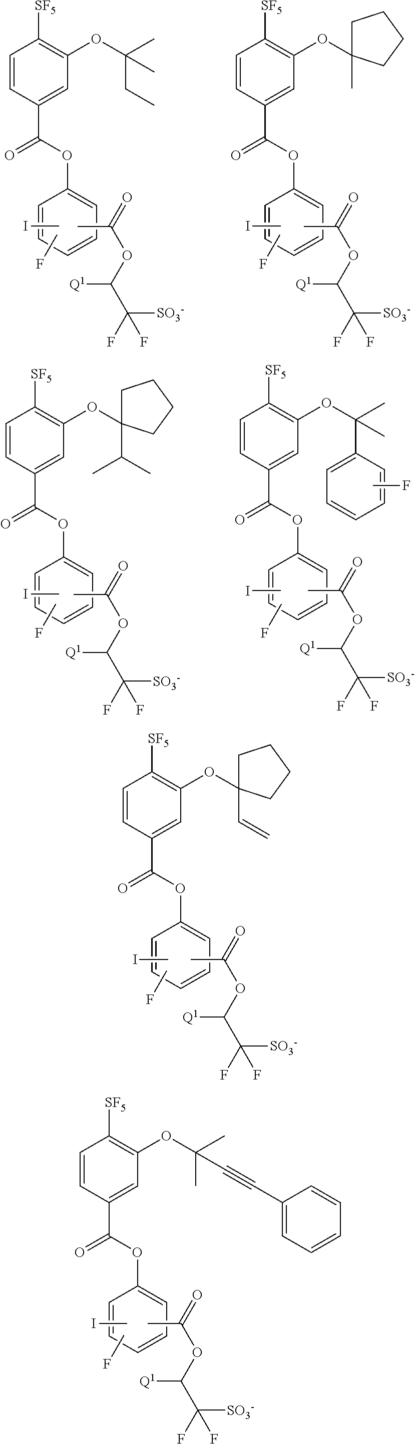

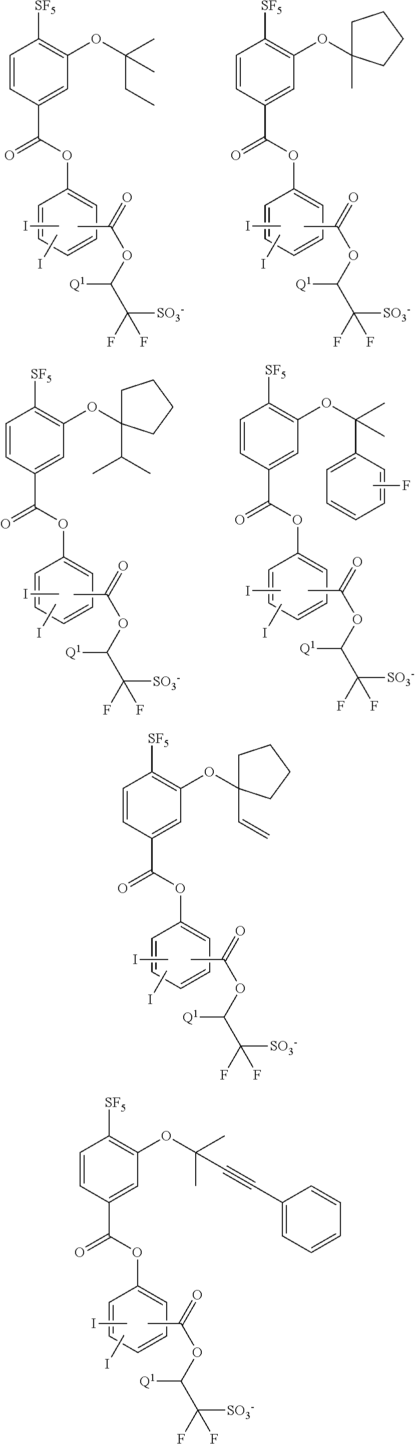

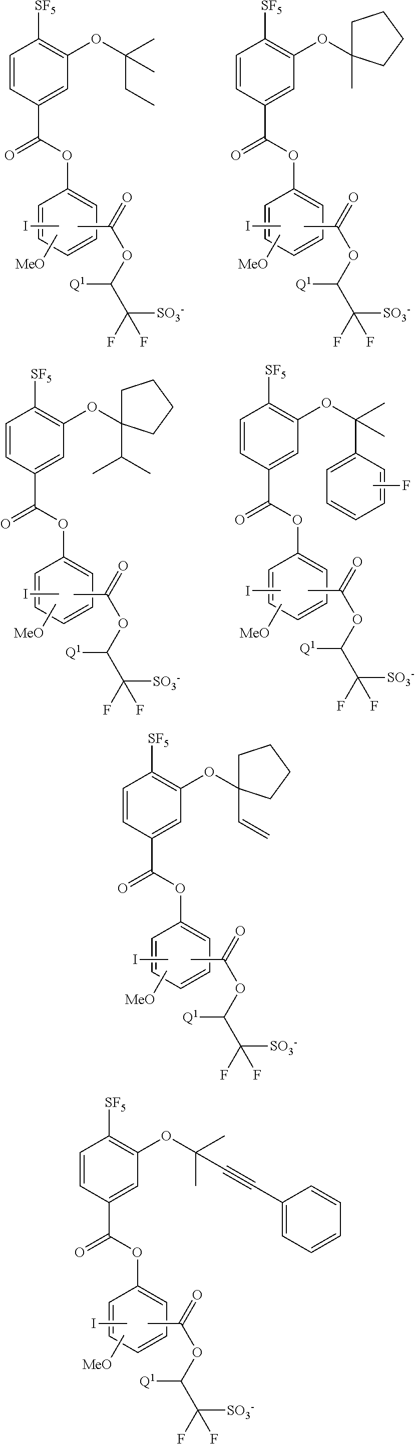

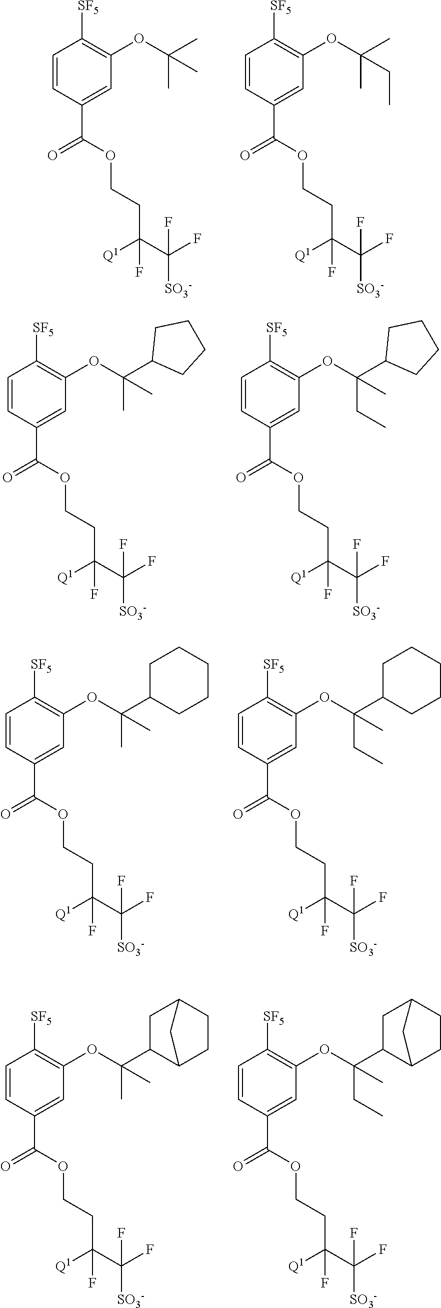

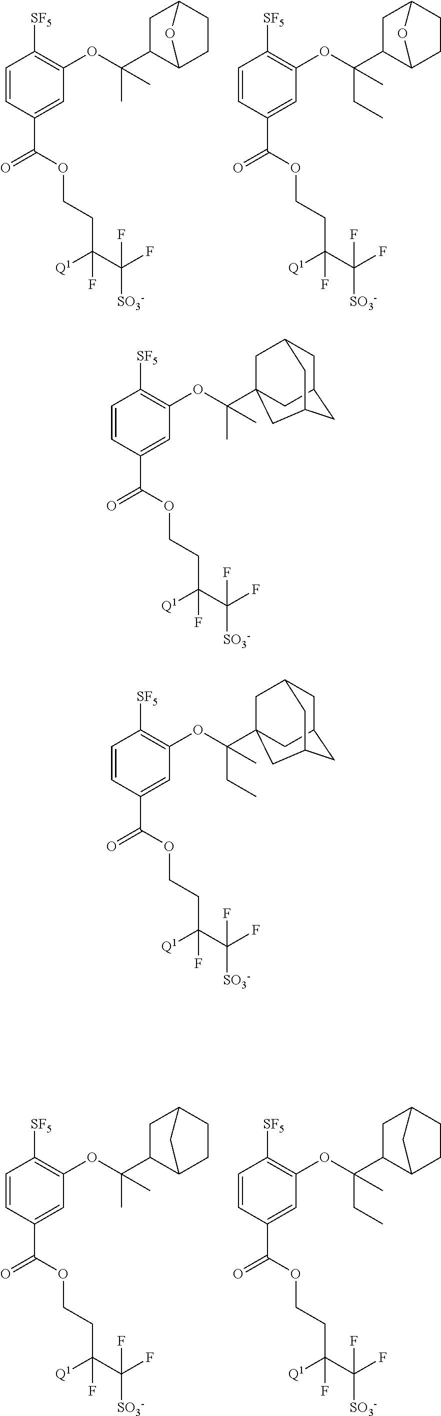

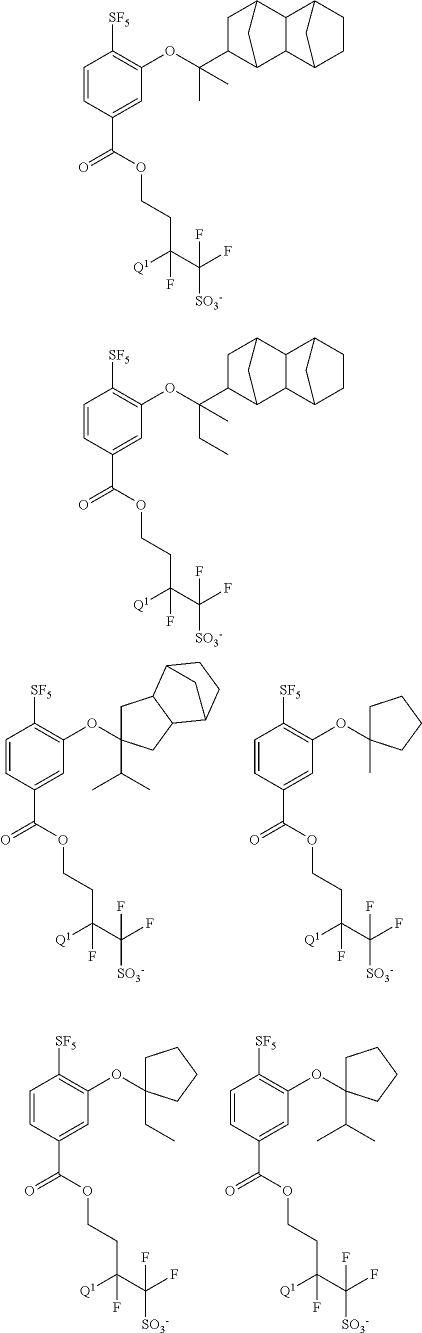

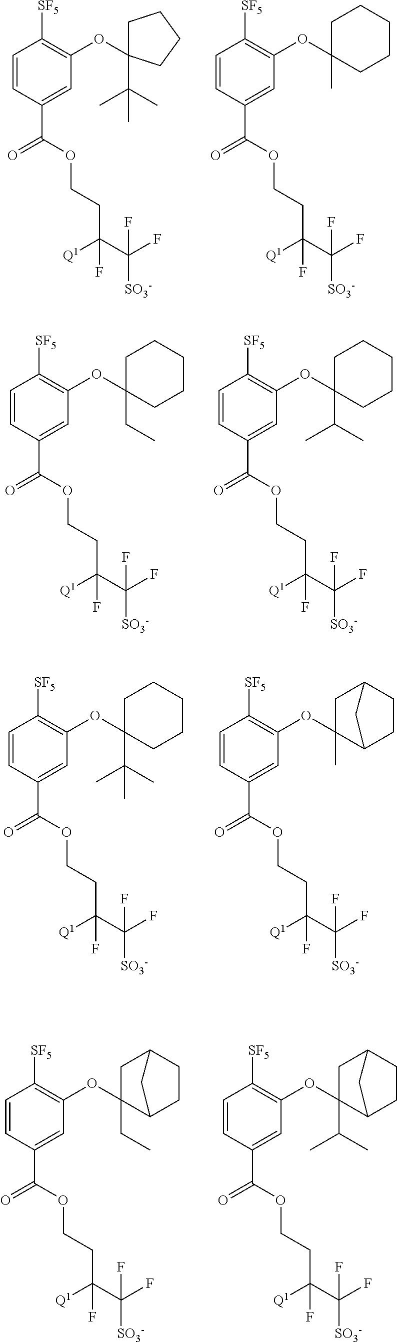

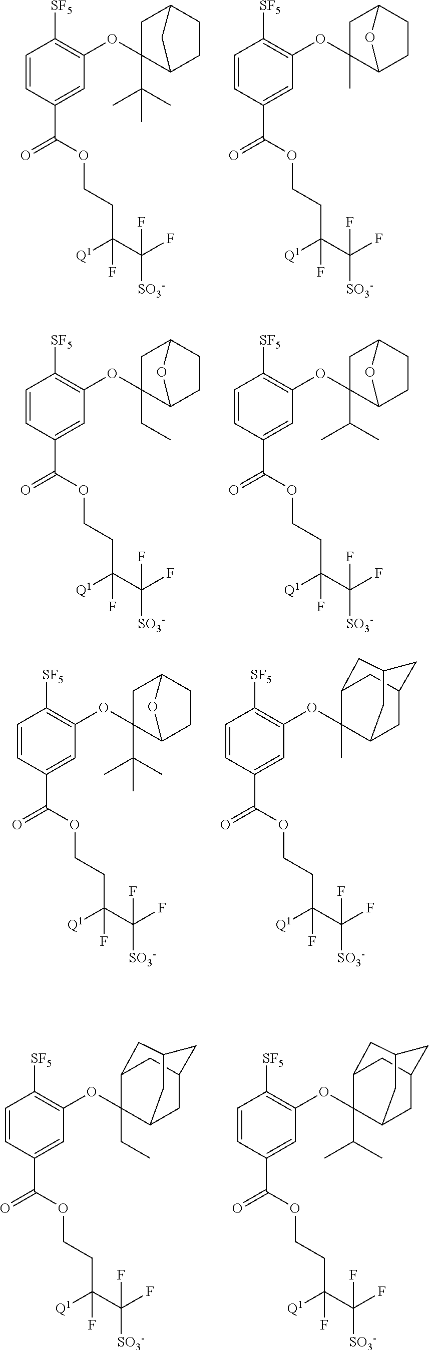

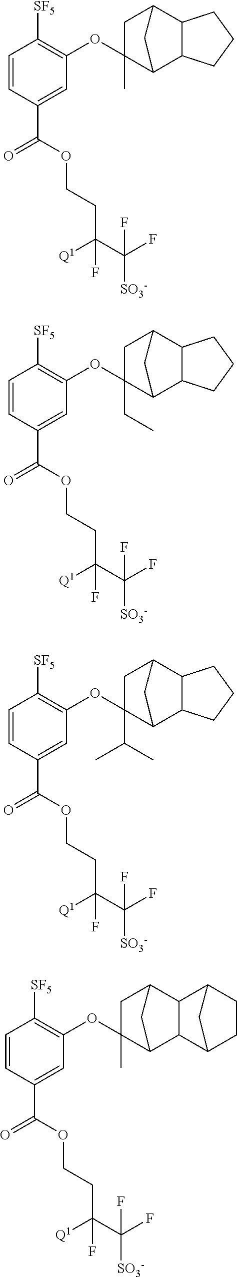

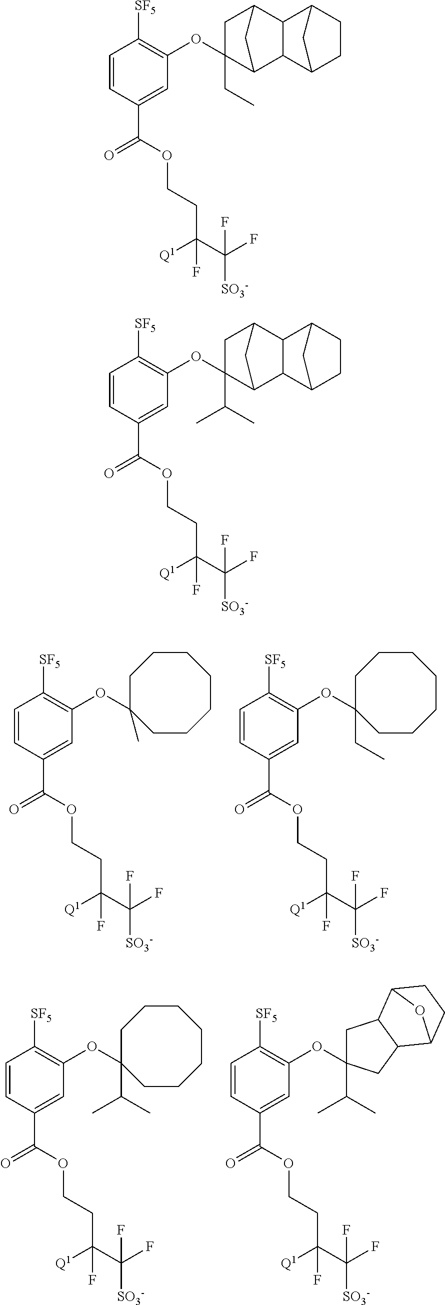

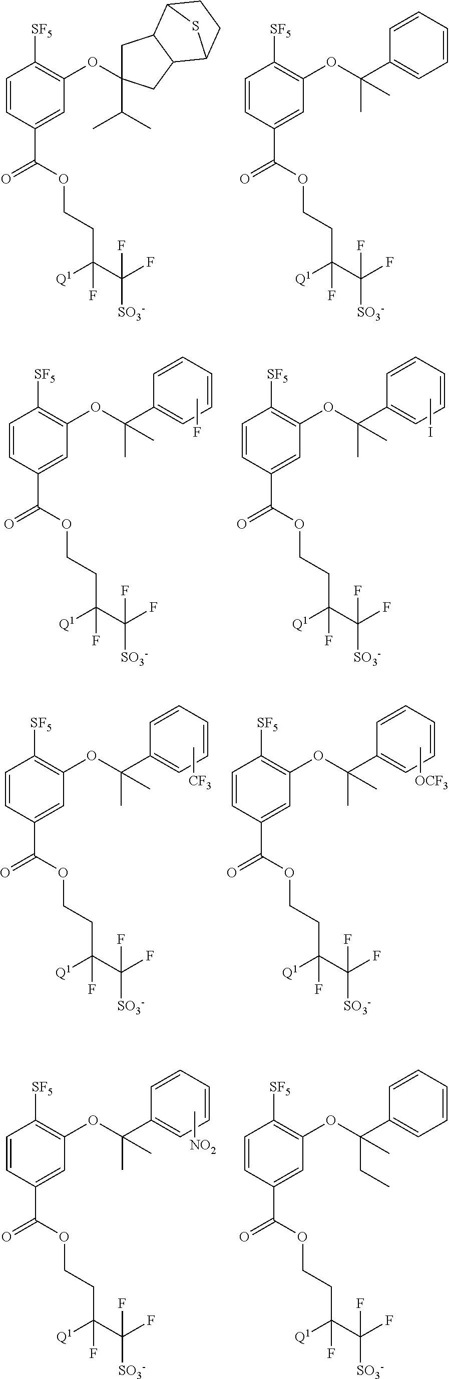

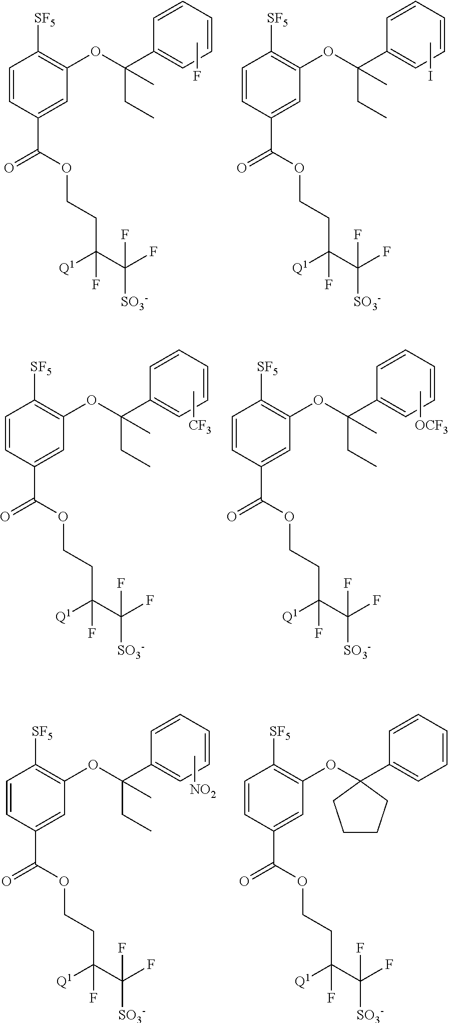

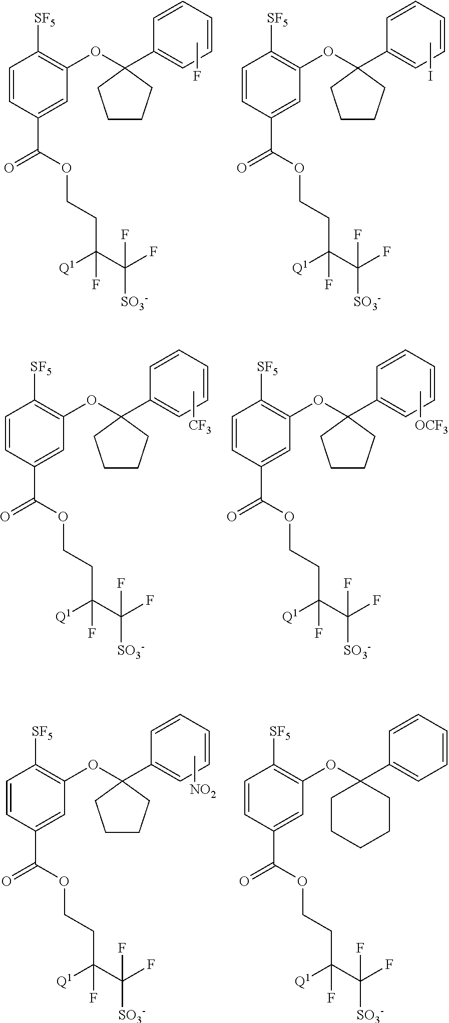

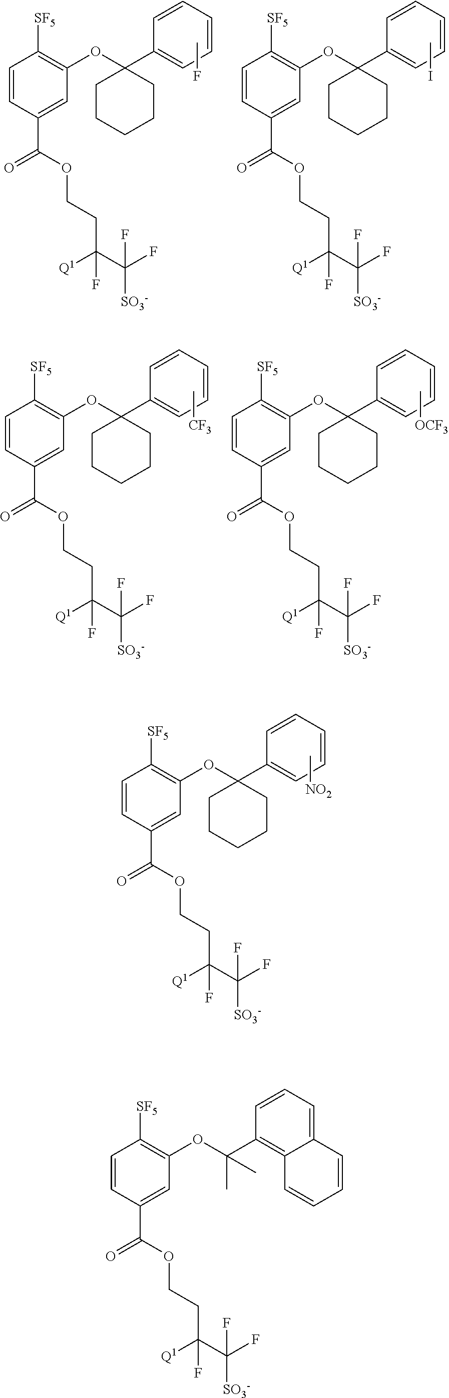

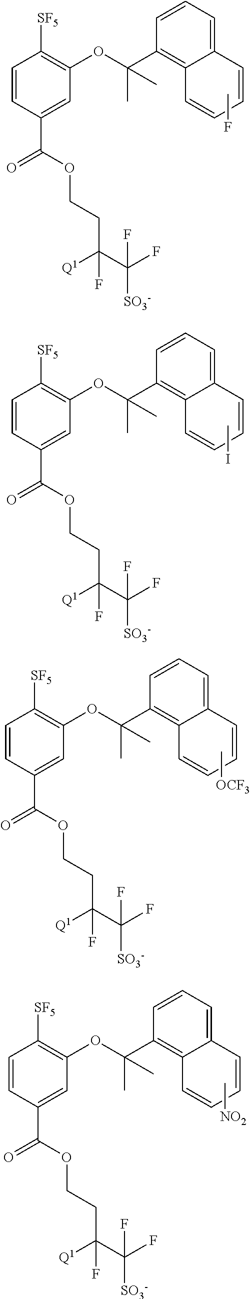

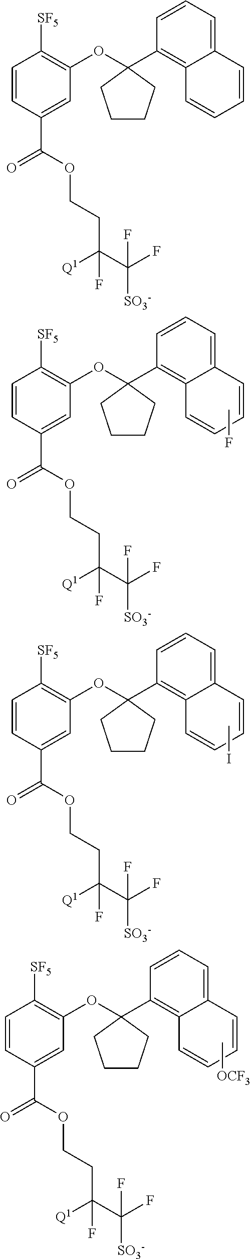

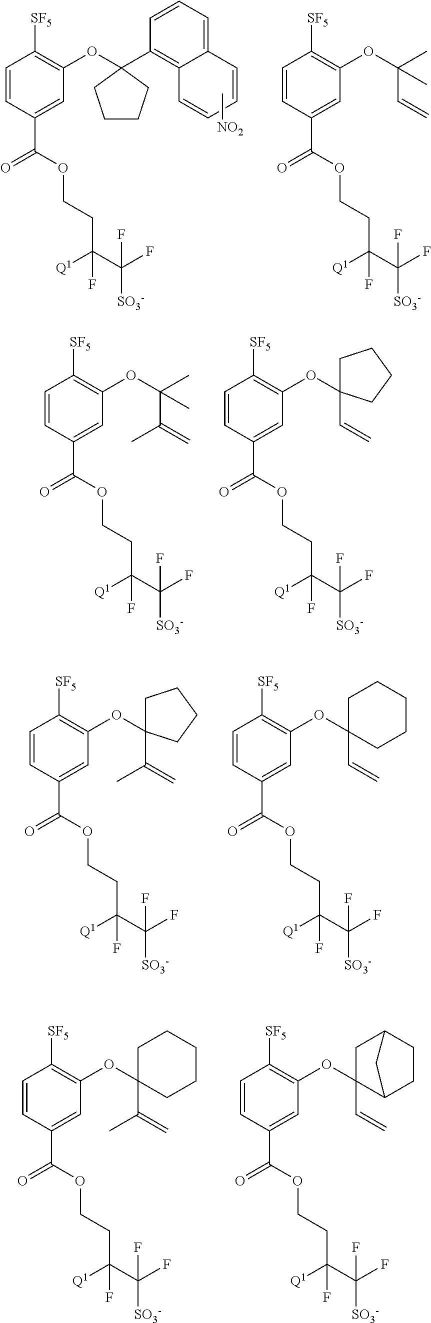

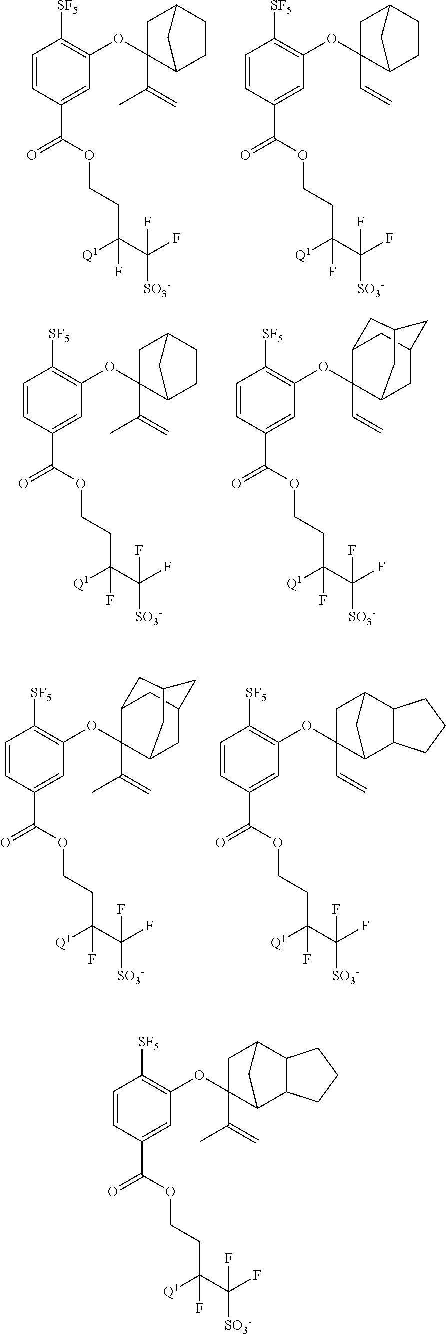

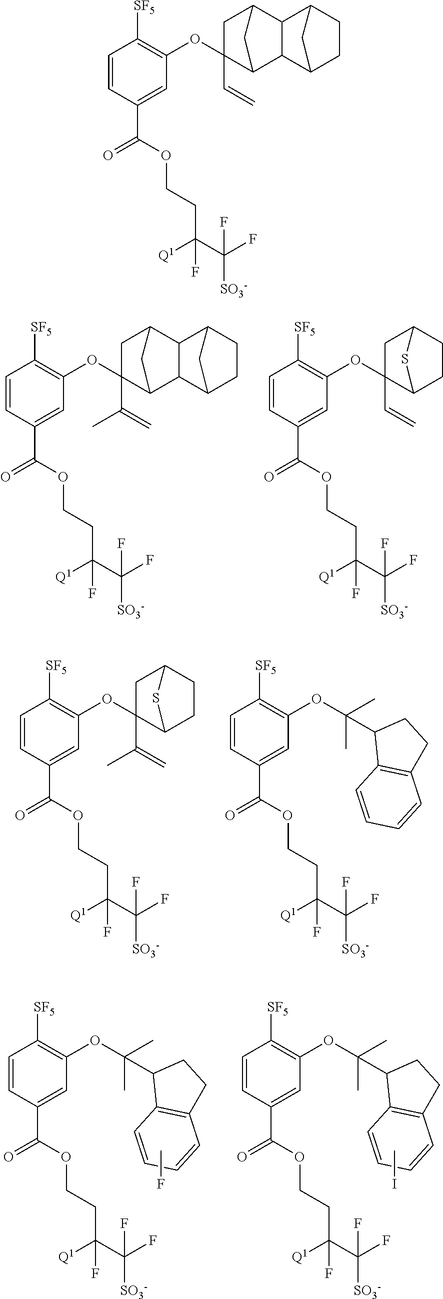

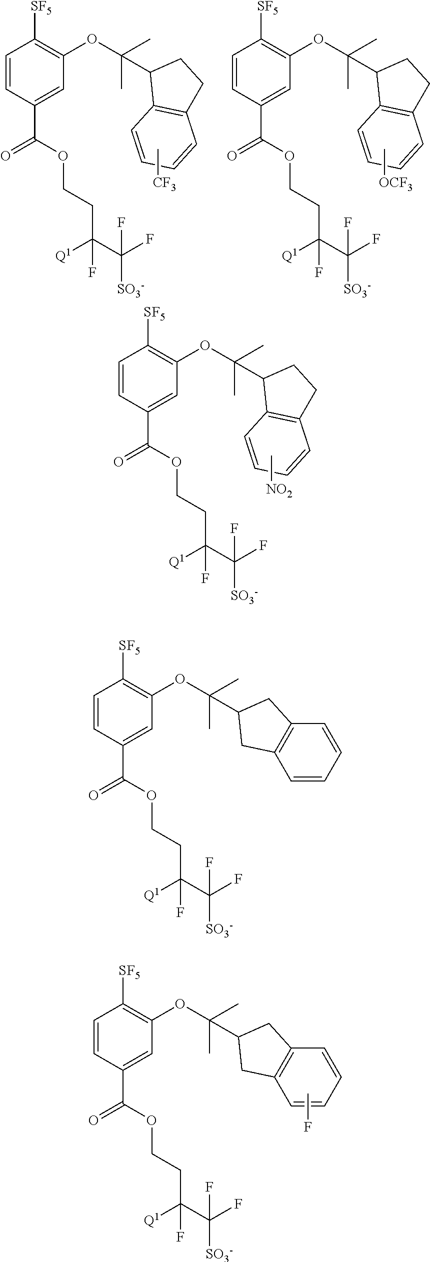

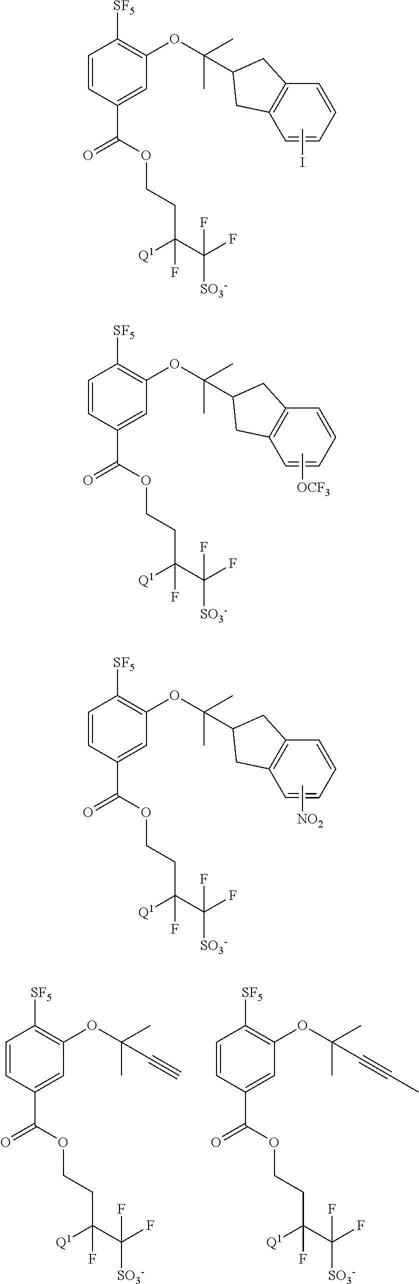

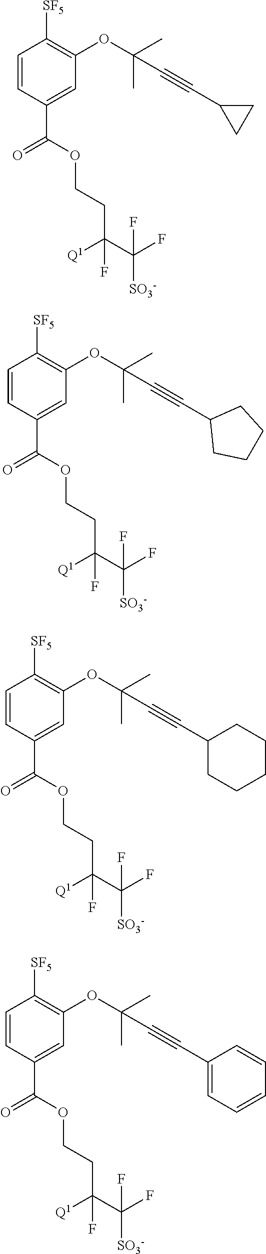

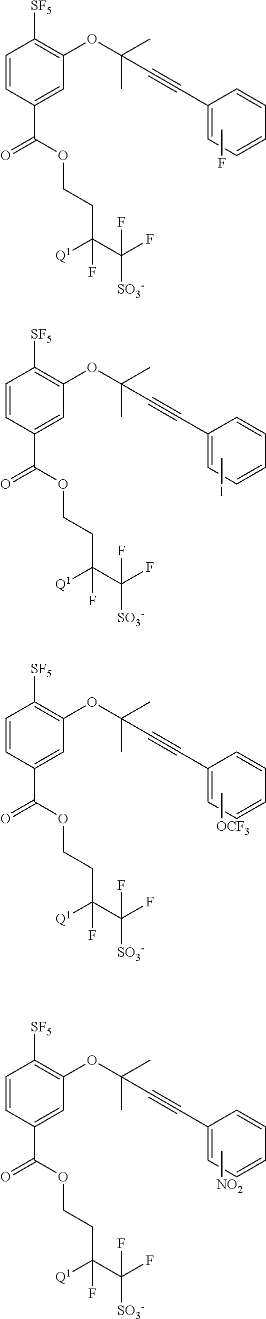

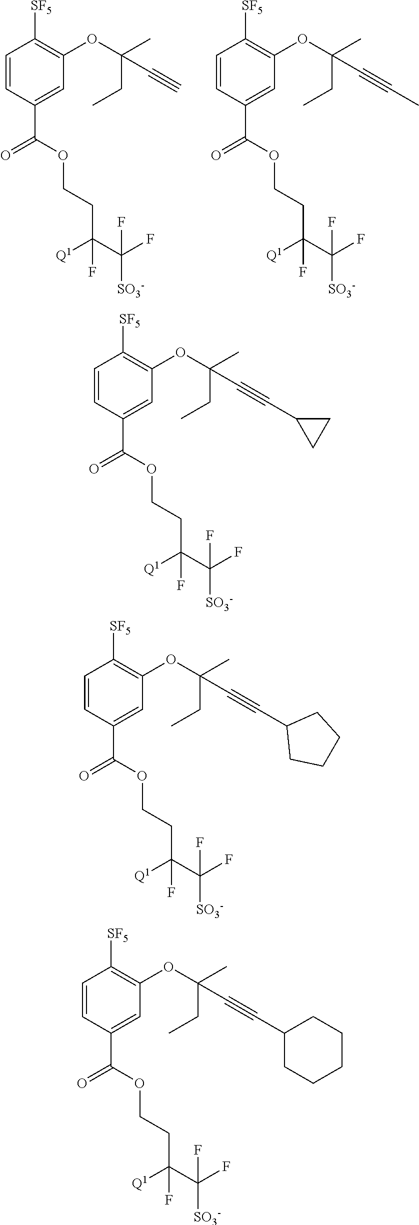

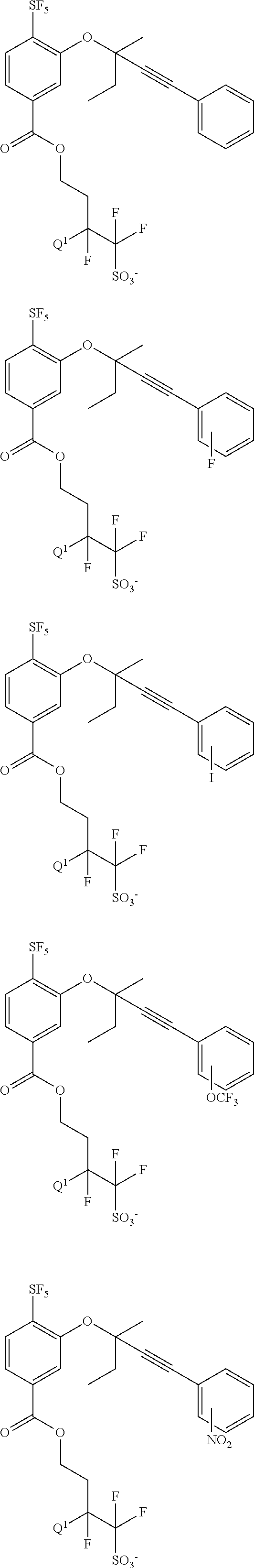

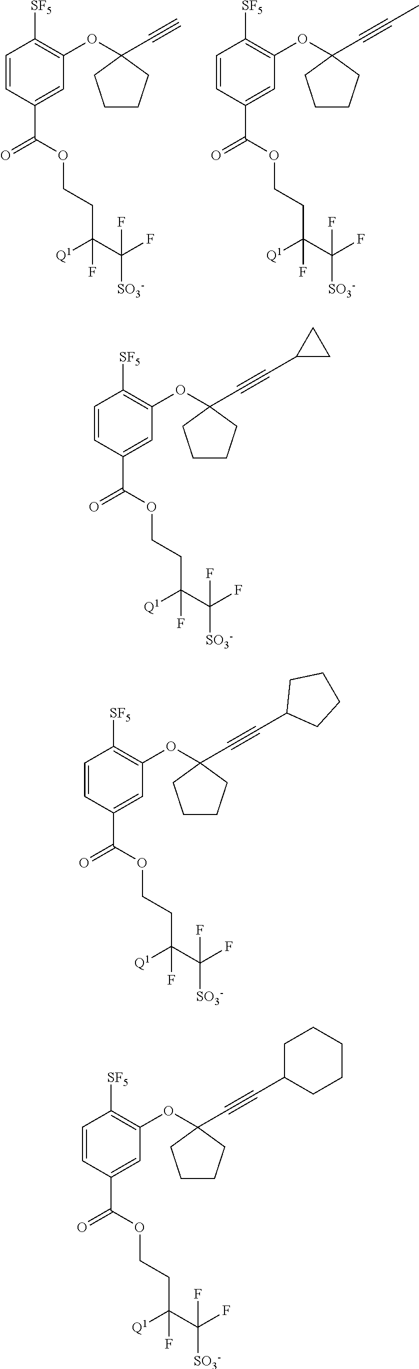

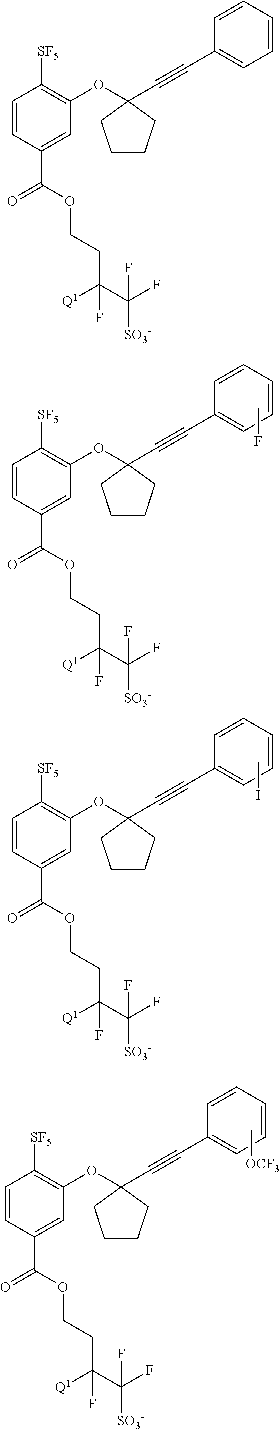

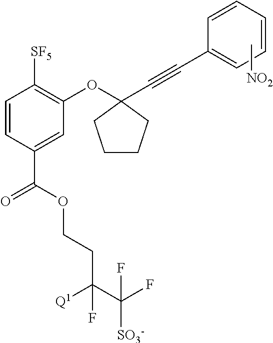

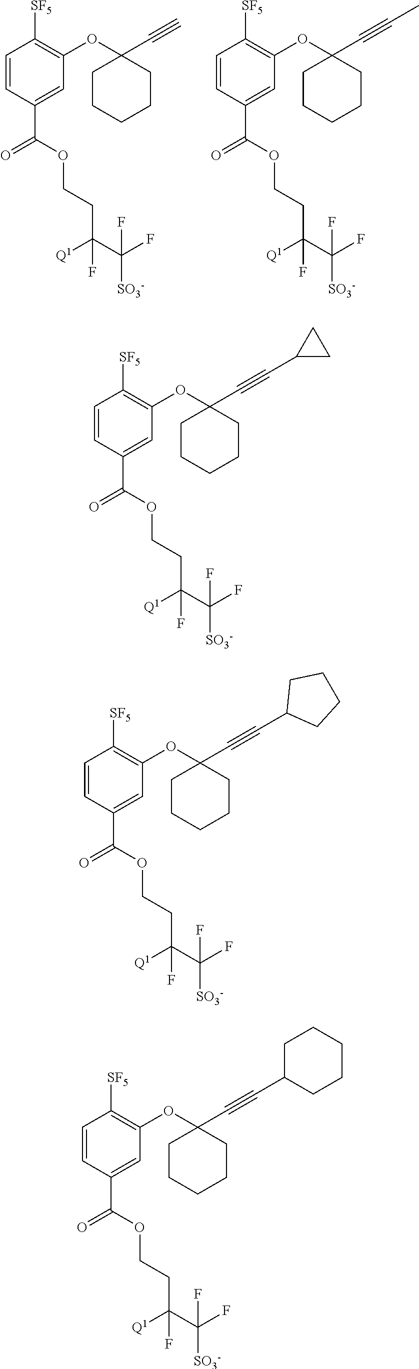

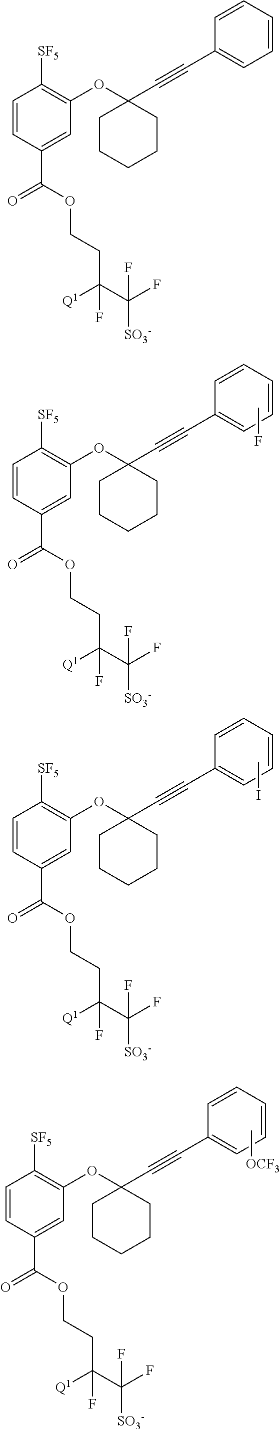

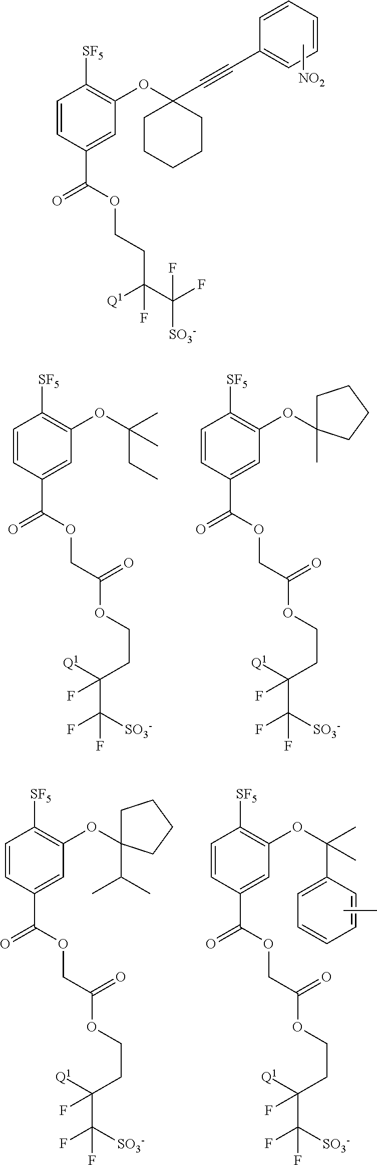

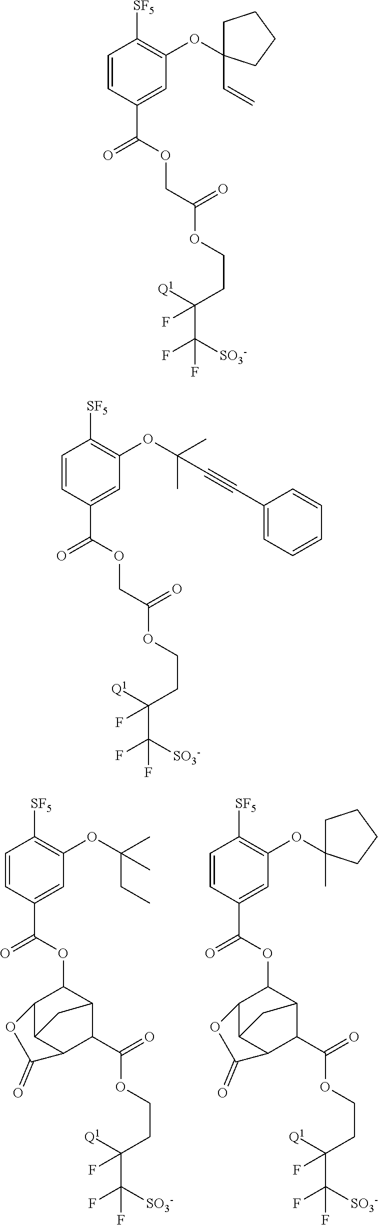

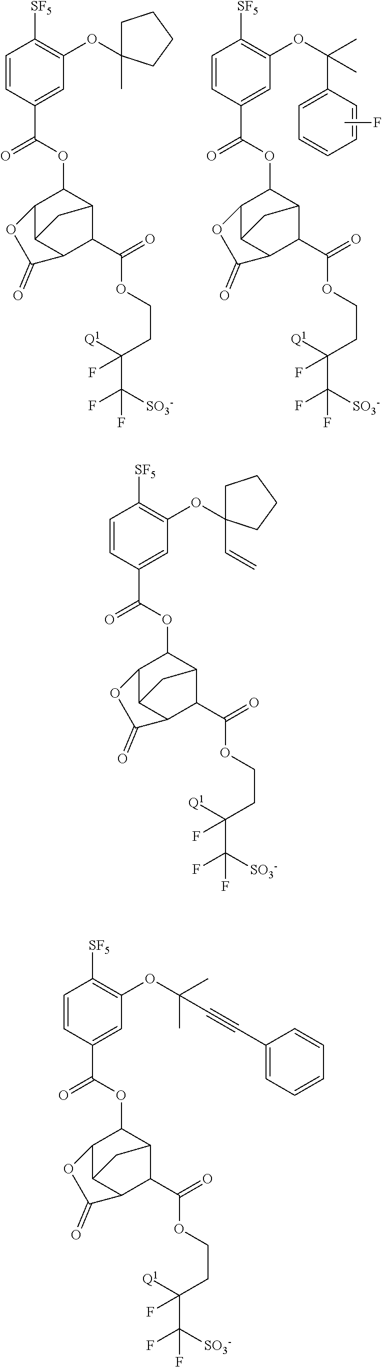

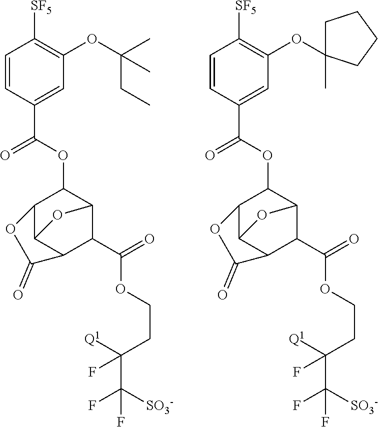

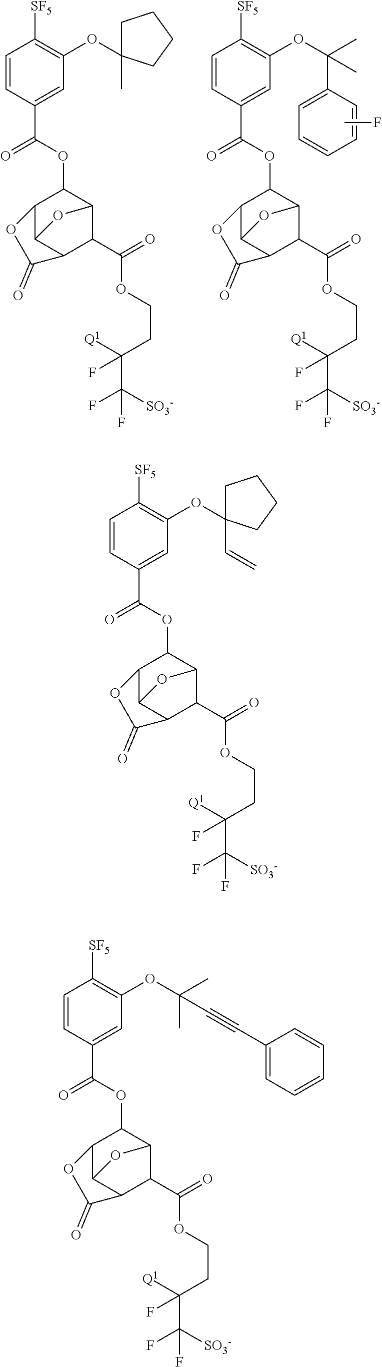

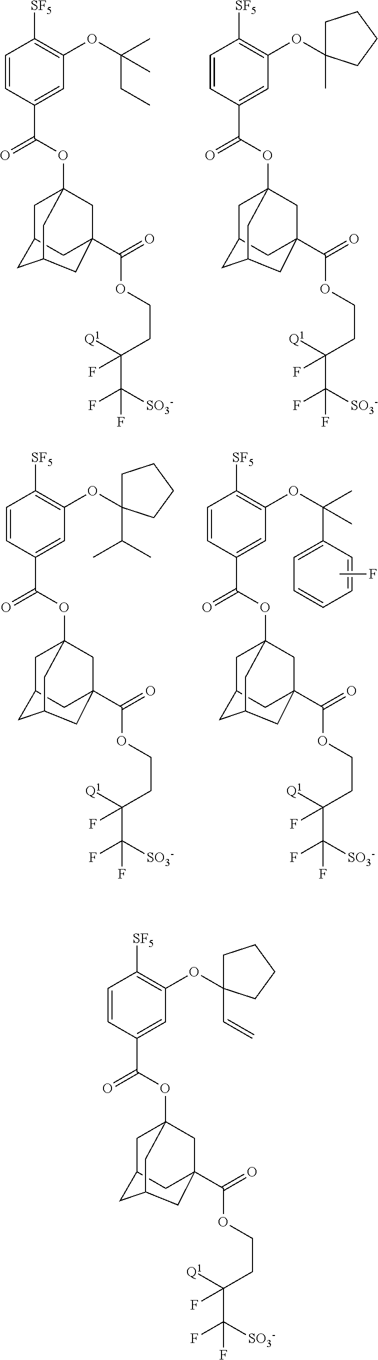

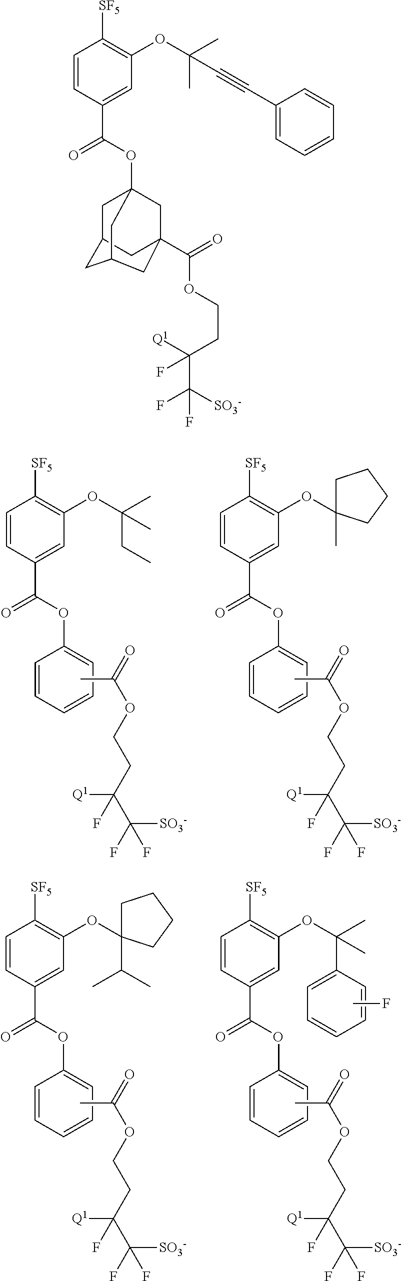

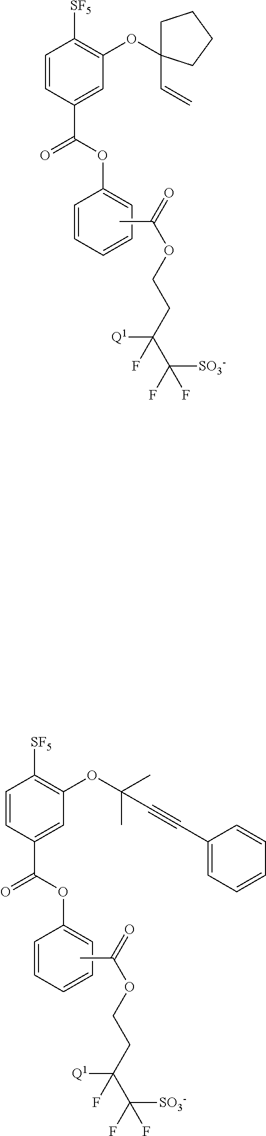

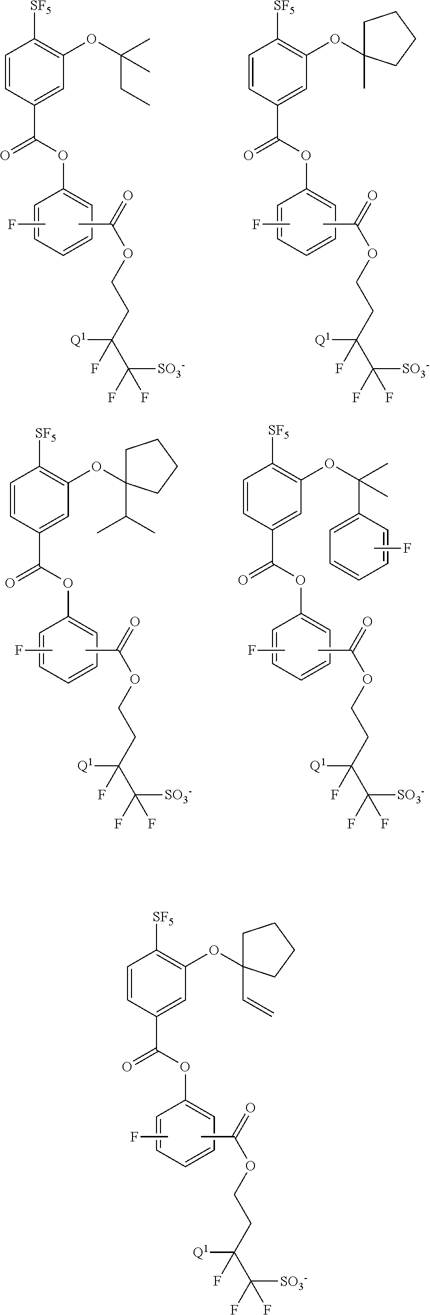

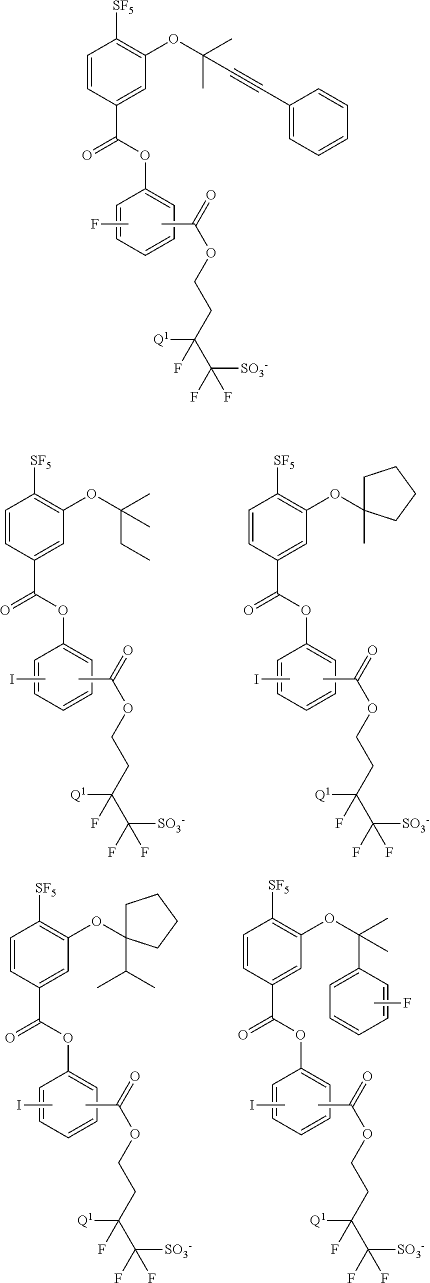

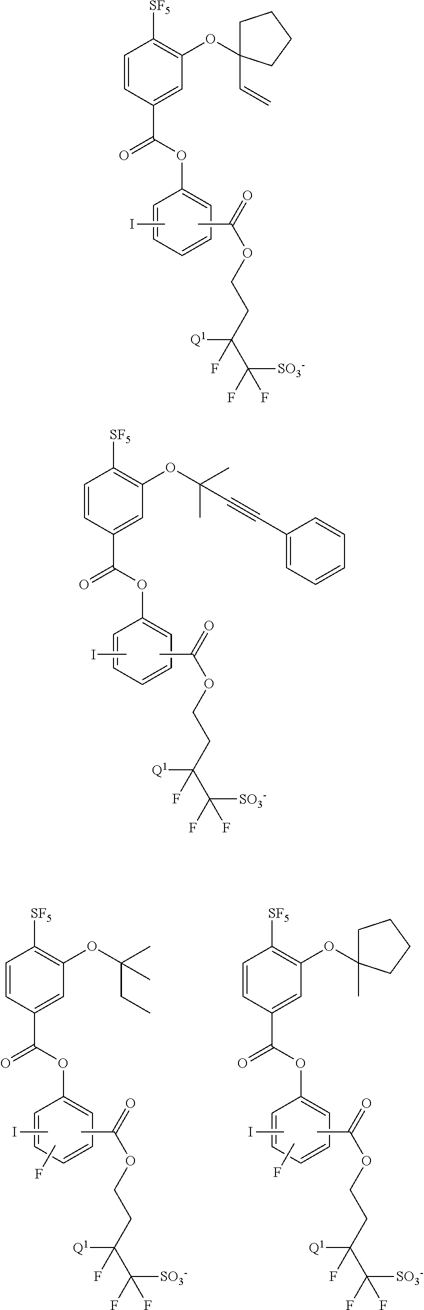

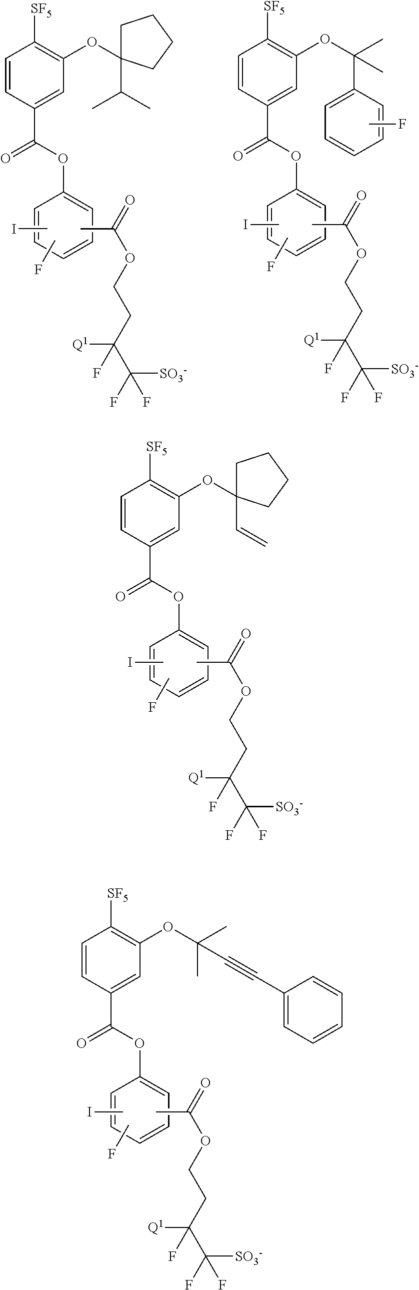

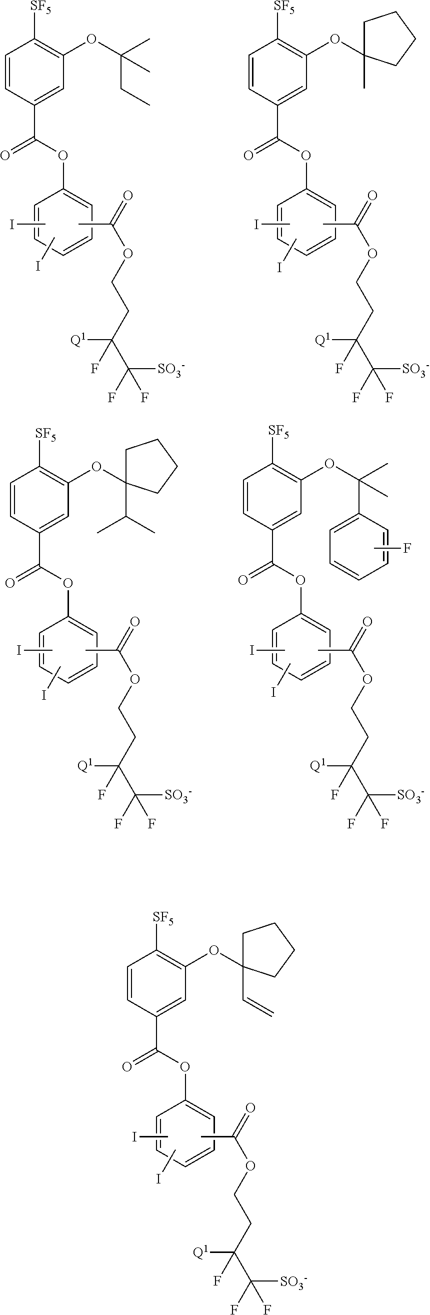

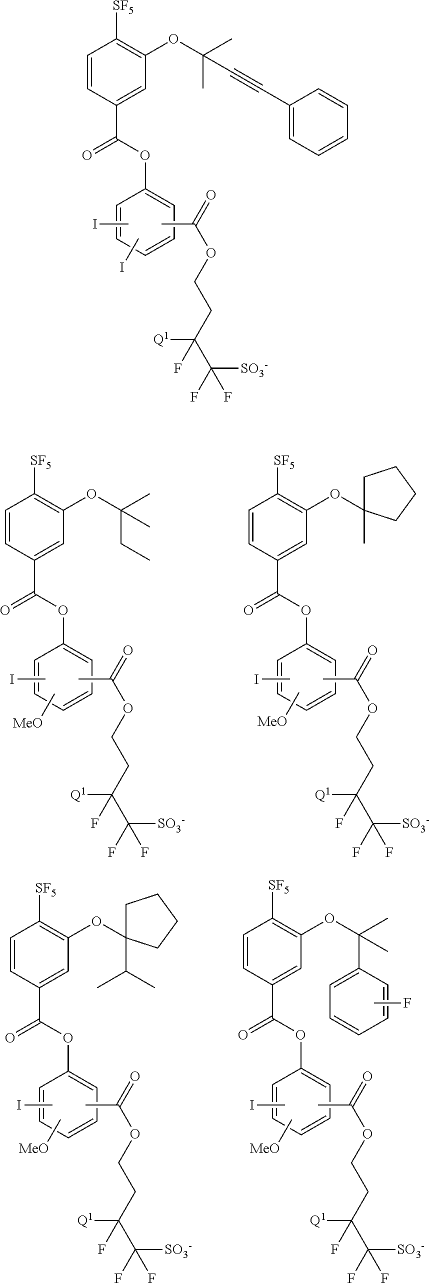

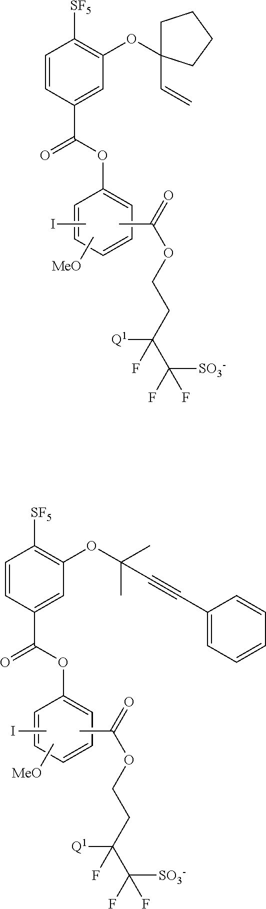

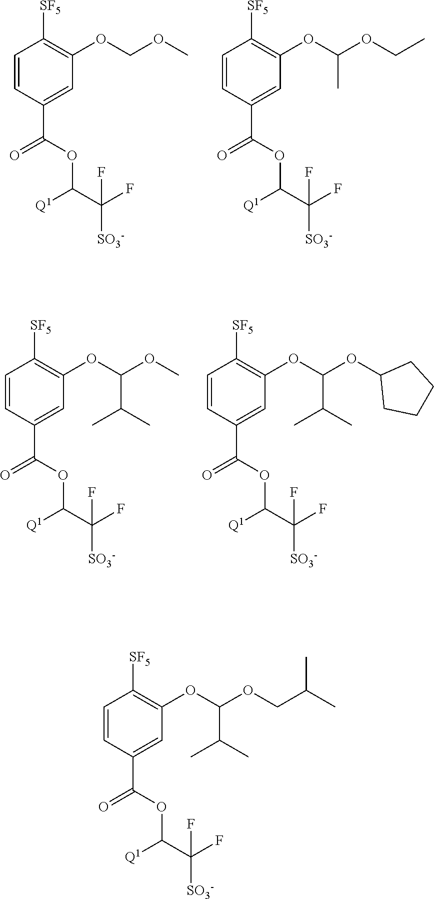

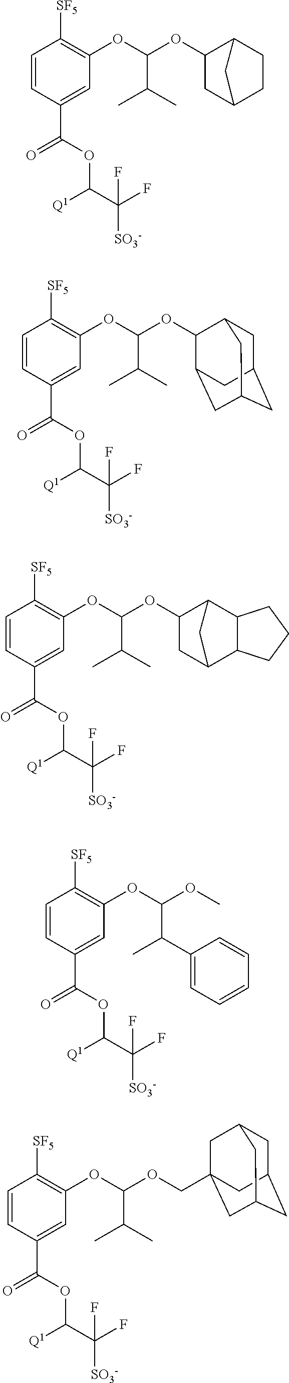

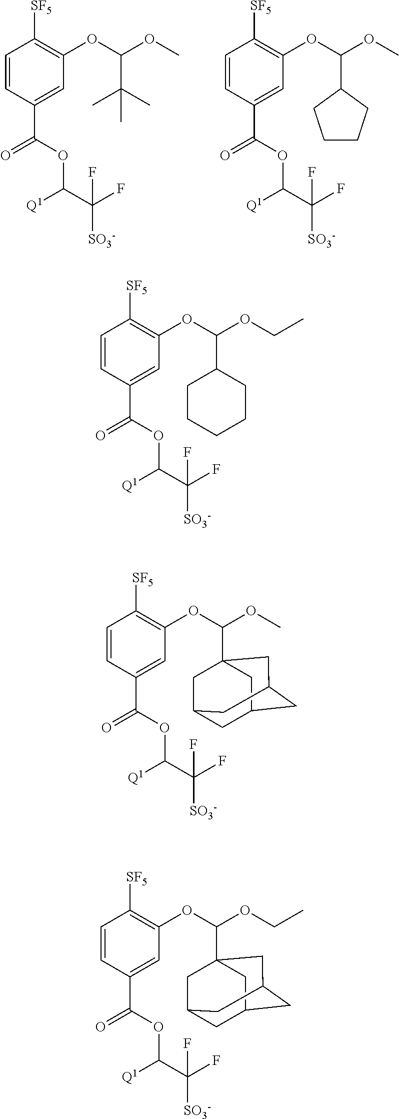

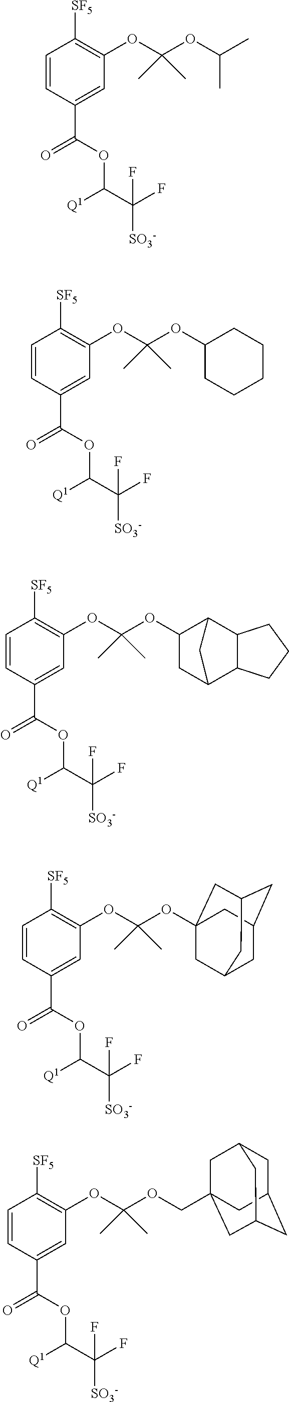

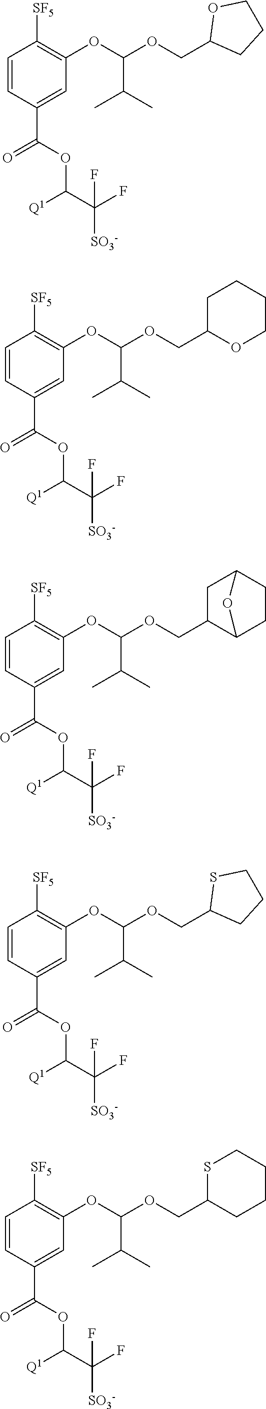

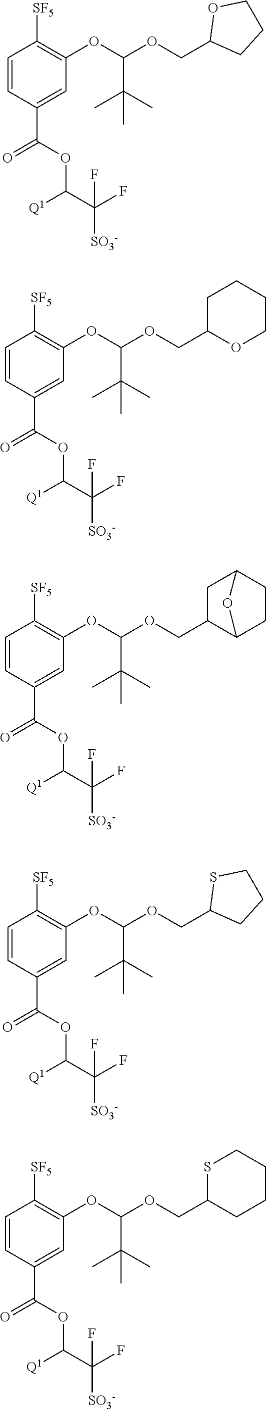

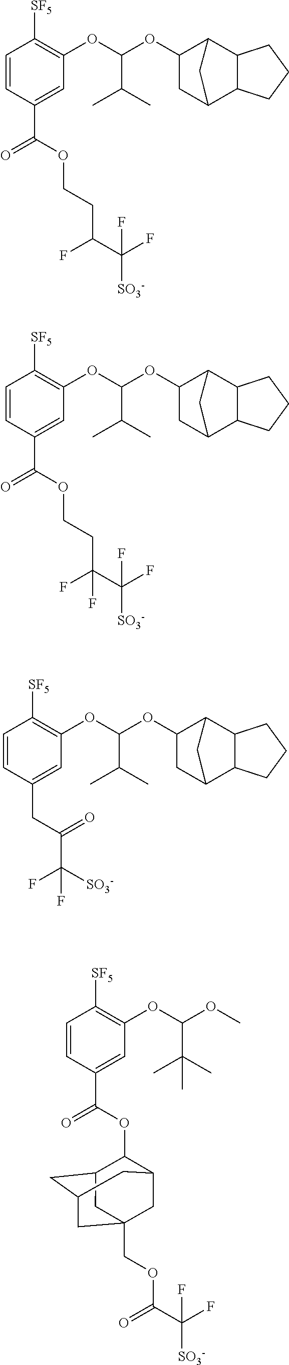

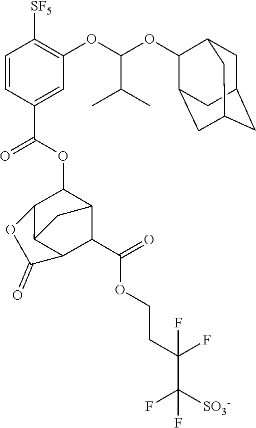

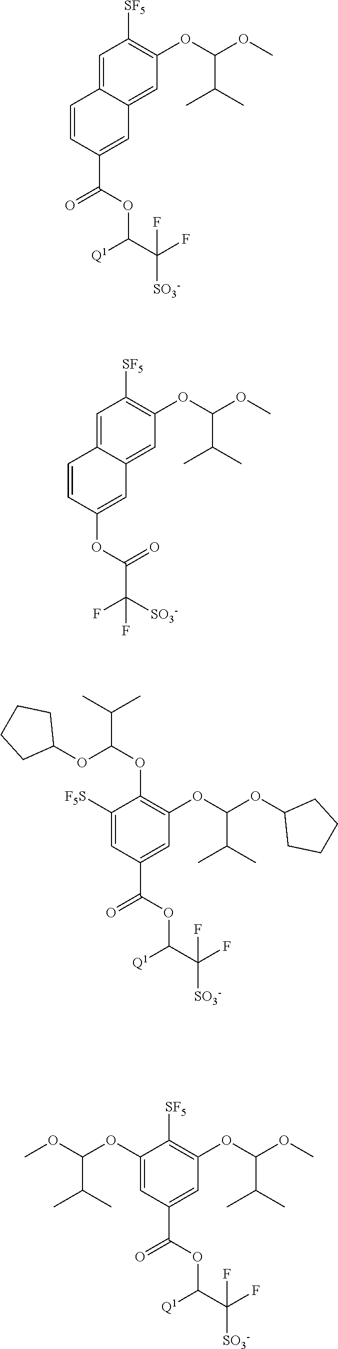

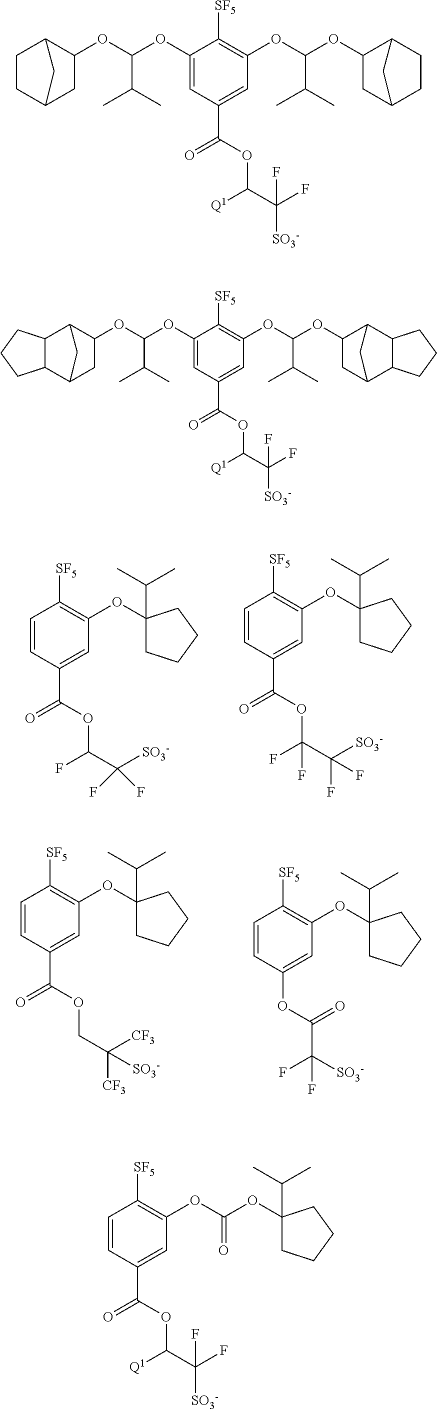

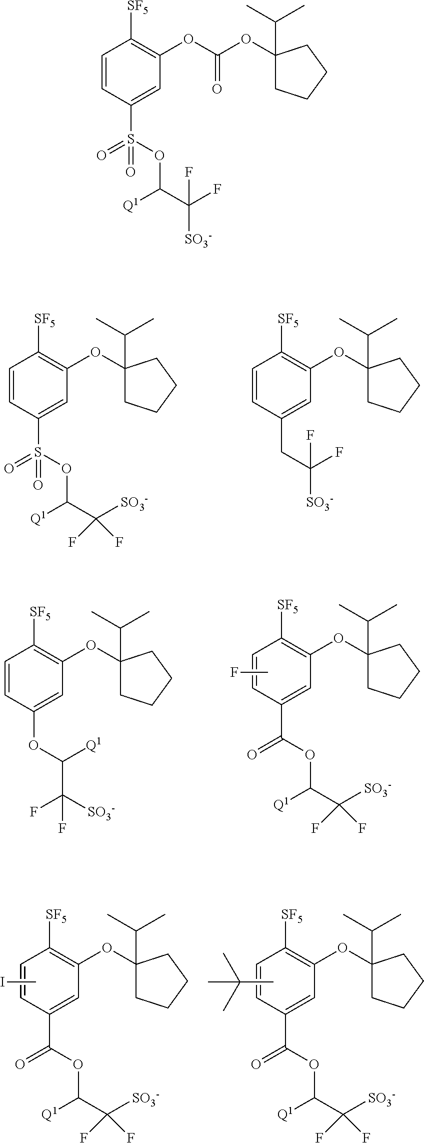

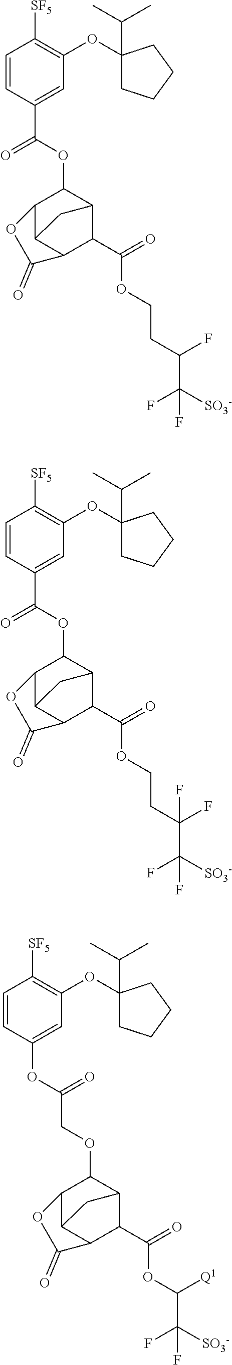

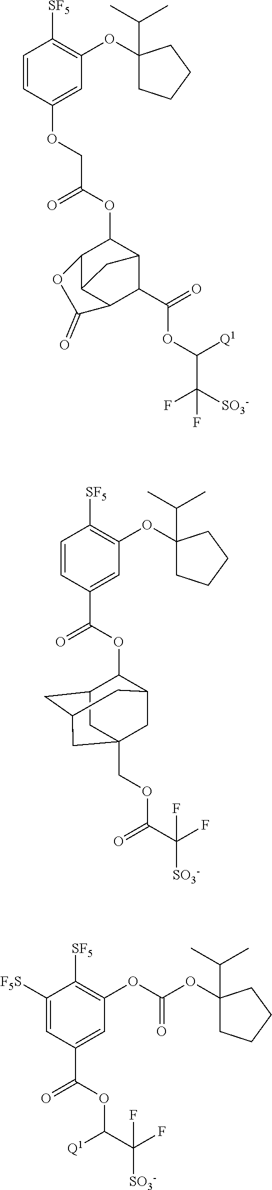

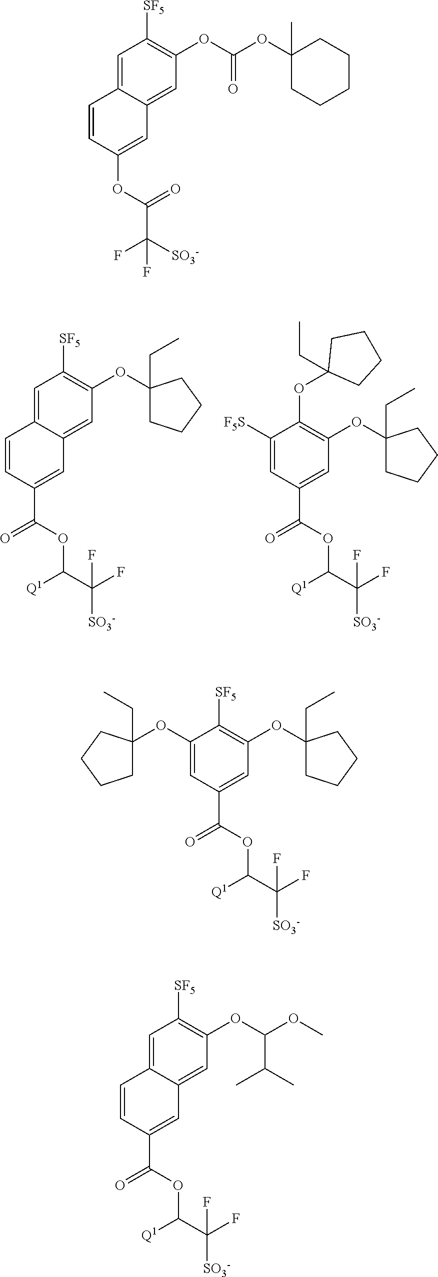

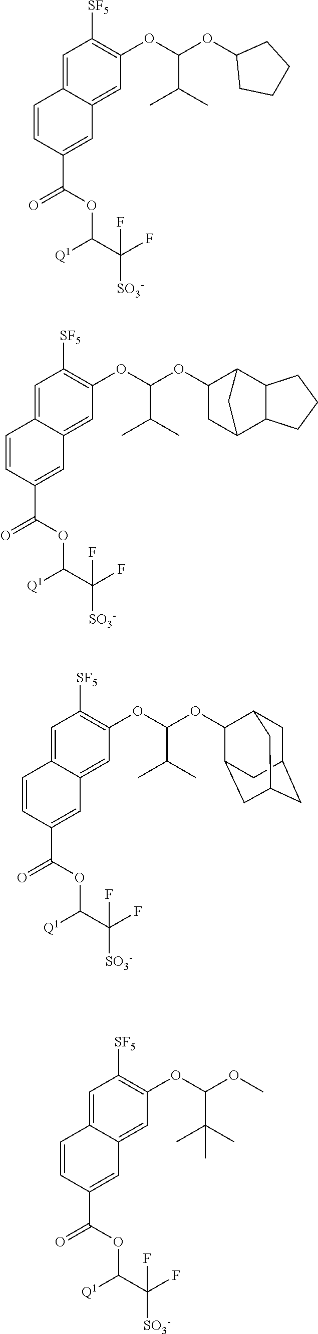

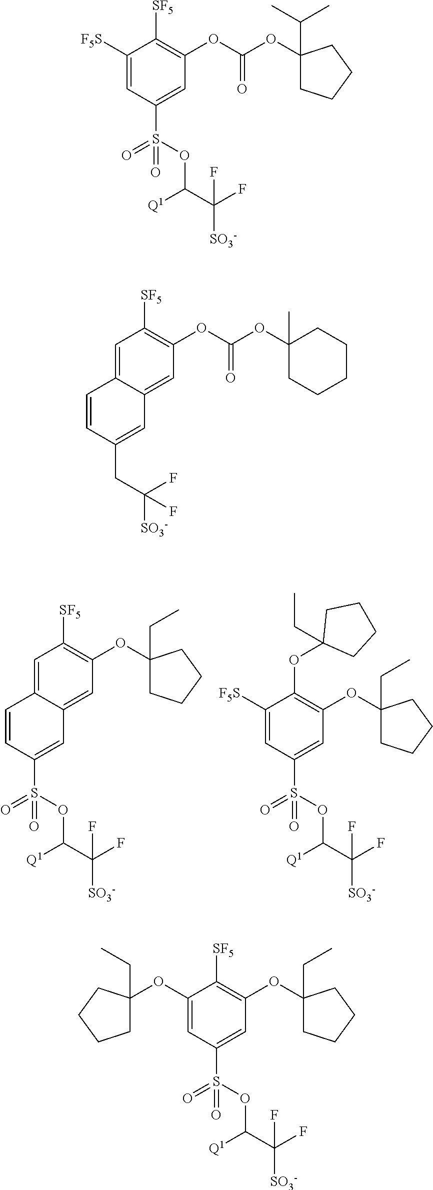

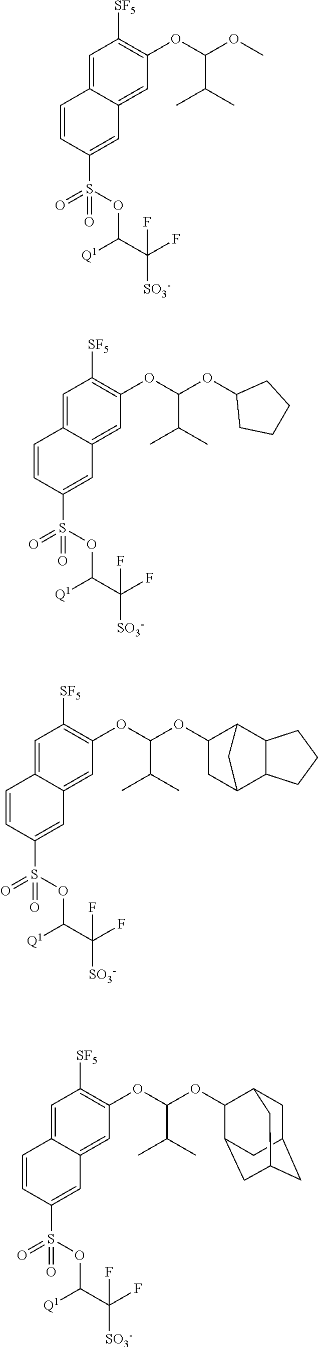

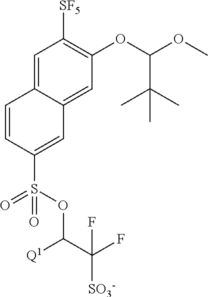

In formula (1), Z⁺ is an onium cation. The onium cation is preferably a sulfonium cation having the formula (Z-1) or an iodonium cation having the formula (Z-2).

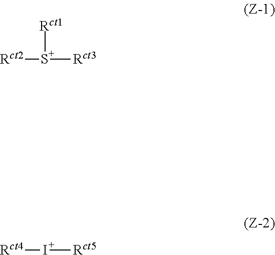

In formulae (Z-1) and (Z-2), R^{ct1} to R^{ct5} are each independently a halogen atom or a C₁-C₃₀ hydrocarbyl group which may contain a heteroatom.

Specific examples of the halogen atom of R^{ct1} to R^{ct5} include a fluorine atom, a chlorine atom, a bromine atom, and an iodine atom.

The hydrocarbyl groups R^{ct1} to R^{ct5} may be saturated or unsaturated and straight, branched, or cyclic. Specific examples thereof include C₁-C₃₀ alkyl groups such as methyl, ethyl, n-propyl, isopropyl, n-butyl, isobutyl, sec-butyl, and tert-butyl; C₃-C₃₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₃₀ alkenyl groups such as vinyl, 1-propenyl, 2-propenyl, butenyl, and hexenyl; C₃-C₃₀ cyclic unsaturated hydrocarbyl groups such as cyclohexenyl; C₆-C₃₀ aryl groups such as phenyl, naphthyl, and thienyl; C₇-C₃₀ aralkyl groups such as benzyl, 1-phenylethyl, and 2-phenylethyl, and combinations thereof, and the aryl groups are preferred. In the hydrocarbyl group, some or all of the hydrogen atoms may be substituted by a moiety containing a heteroatom such as an oxygen atom, a sulfur atom, a nitrogen atom, or a halogen atom, and some constituent —CH₂— may be replaced by a moiety containing a heteroatom such as an oxygen atom, a sulfur atom, or a nitrogen atom, so that the group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—) or a haloalkyl group.

R^{ct1} and R^{ct2} may bond together to form a ring with the sulfur atom to which they are attached. Specific examples of the structure of the ring are shown below.

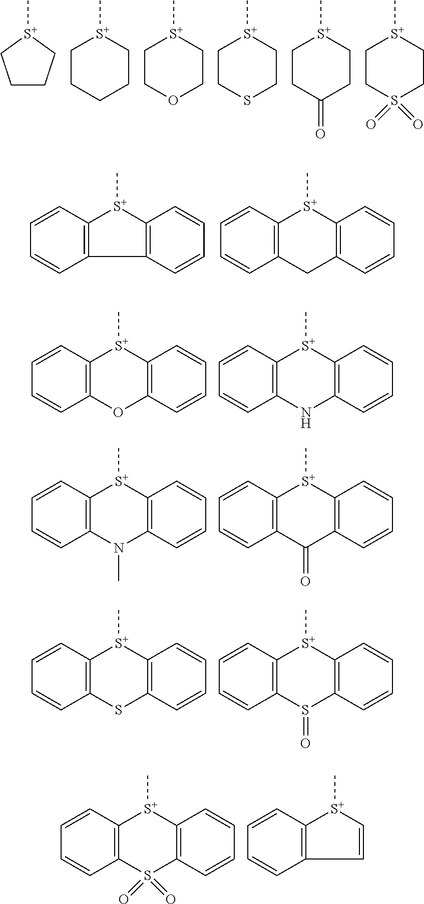

Herein the broken line designates a point of attachment to R^{ct3}.

Specific examples of the sulfonium cation having formula (Z-1) are shown in paragraphs to of JP-A 2024-003744 and paragraphs to of JP-A 2023-169812, but not limited thereto.

Specific examples of the iodonium cation of the formula (Z-2) are shown in paragraph of JP-A 2024-000259, but not limited thereto.

As the onium cation of Z⁺, a sulfonium cation having the formula (Z-3) is also preferred.

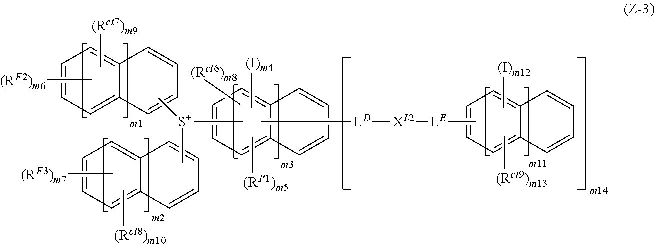

In formula (Z-3), m1 is 0 or 1. The relevant structure is a benzene ring when m1 is 0, and a naphthalene ring when m1 is 1. From the viewpoint of solvent solubility, the benzene ring corresponding to m1=0 is preferred. m2 is 0 or 1. The relevant structure is a benzene ring when m2 is 0, and a naphthalene ring when m2 is 1. From the viewpoint of solvent solubility, the benzene ring corresponding to m2=0 is preferred. m3 is 0 or 1. The relevant structure is a benzene ring when m3 is 0, and a naphthalene ring when m3 is 1. From the viewpoint of solvent solubility, the benzene ring corresponding to m3=0 is preferred.

In formula (Z-3), m4 is 0, 1, 2, 3, or 4. The larger the number of iodine atoms in the cationic structure is, the more the absorption for EUV is particularly enhanced, but since there is a concern that the solvent solubility is reduced and precipitation occurs in the resist composition, m4 is preferably 0, 1, 2, or 3, more preferably 0, 1, or 2.

In formula (Z-3), m5 is 0, 1, 2, 3, or 4. From the viewpoint of reactant availability, m5 is preferably 0, 1, 2, or 3, more preferably 0, 1, or 2. m6 is 0, 1, 2, 3, 4, 5, or 6. From the viewpoint of reactant availability, m6 is preferably 0, 1, 2, or 3, more preferably 0, 1, or 2. m7 is 0, 1, 2, 3, 4, 5, or 6. From the viewpoint of reactant availability, m7 is preferably 0, 1, 2, or 3, more preferably 0, 1, or 2.

In formula (Z-3), m8 is 0, 1, or 2. From the viewpoint of reactant availability, m8 is preferably 0 or 1. m9 is 0, 1, or 2. From the viewpoint of reactant availability, m9 is preferably 0 or 1. m10 is 0, 1, or 2. From the viewpoint of reactant availability, m10 is preferably 0 or 1.

In formula (Z-3), m11 is 0 or 1. The relevant structure is a benzene ring when m11 is 0, and a naphthalene ring when m11 is 1. From the viewpoint of solvent solubility, the benzene ring corresponding to m11=0 is preferred.

In formula (Z-3), m12 is 0, 1, 2, 3, or 4. The larger the number of iodine atoms in the cationic structure is, the more the absorption for EUV is particularly enhanced, but since there is a concern that the solvent solubility is reduced and precipitation occurs in the resist composition, m12 is preferably 0, 1, 2, or 3, more preferably 0, 1, or 2.

In formula (Z-3), m13 is 0, 1, or 2. From the viewpoint of reactant availability, m13 is preferably 0 or 1. m14 is 0, 1, or 2. From the viewpoint of synthesis, m14 is preferably 0 or 1.

Provided that 0≤m6+m9≤4 when m1 is 0, and 0≤m6+m9≤6 when m1 is 1.0≤m7+m10≤4 when m2 is 0, and 0 $ m7+m10≤6 when m2 is 1. 1≤m4+m5+m8+m14 ≤4 when m3 is 0, and 1≤m4+m5+m8+m14≤6 when m3 is 1. 0≤m12+m13≤4 when m11 is 0, and 0≤m12+m13≤6 when m11 is 1. m4+m12≥1.

In formula (Z-3), R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Inter alia, a trifluoromethyl group, a trifluoromethoxy group, and a trifluorothiomethoxy group are preferred. When m5 is 2 or more, R^{F1} may be identical or different, when m6 is 2 or more, R^{F2} may be identical or different, and when m7 is 2 or more, R^{F3} may be identical or different.

In formula (Z-3), R^{ct6} to R^{ct9} are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and the hydrocarbyl moiety of the hydrocarbyloxy group and the hydrocarbylthio group may be saturated or unsaturated and straight, branched, or cyclic. Specific examples thereof are as exemplified above for the hydrocarbyl group R¹ in the description of formula (1). In the hydrocarbyl group and the hydrocarbyl moiety of the hydrocarbyloxy group and the hydrocarbylthio group, some or all of the hydrogen atoms may be substituted by a moiety containing a heteroatom such as an oxygen atom, a sulfur atom, a nitrogen atom, or a halogen atom, and some constituent —CH₂— may be replaced by a moiety containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

When m8 is 2, two R^{ct6} may be identical or different and two R^{ct6} may bond together to form a ring with the carbon atom to which they are attached, when m9 is 2, two R^{ct7} may be identical or different and two R^{ct7} may bond together to form a ring with the carbon atom to which they are attached, when m10 is 2, two R^{ct8} may be identical or different and two R^{ct8} may bond together to form a ring with the carbon atom to which they are attached, and when m13 is 2, two R^{ct9} may be identical or different and two R^{ct9} may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. In the ring, some or all of the hydrogen atoms may be substituted by a moiety containing a heteroatom such as an oxygen atom, a sulfur atom, a nitrogen atom, or a halogen atom, and some constituent —CH₂— may be replaced by a moiety containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

Aromatic rings directly bonded to S⁺ in the sulfonium cation having formula (Z-3) may bond together to form a ring with S⁺. Specific examples of the structure of the ring are shown below.

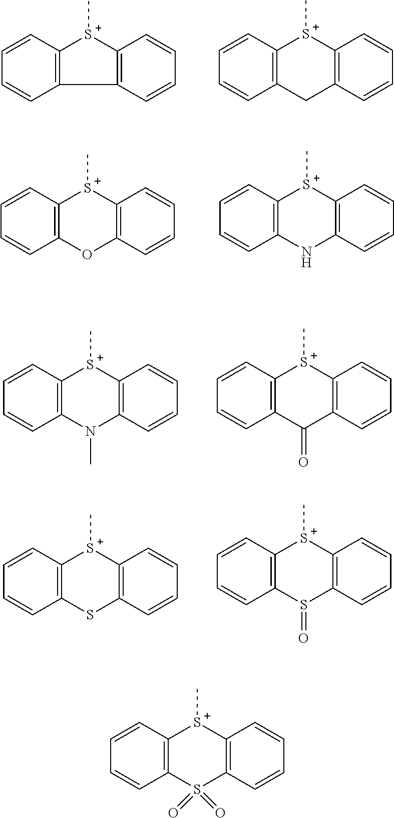

Herein the broken line designates a point of attachment.

In formula (Z-3), L^{D} and L^{E} are each independently a single bond, an ether bond, an ester bond, an amide bond, a sulfonate ester bond, a sulfonamide bond, a carbonate bond, or a carbamate bond. Inter alia, LP is preferably a single bond, an ether bond, an ester bond, or a sulfonate ester bond, more preferably an ester bond or a sulfonate ester bond. L^{E} is preferably a single bond, an ether bond, or an ester bond, more preferably a single bond.

In formula (Z-3), X¹² is a single bond or a C₁-C₄₀ hydrocarbylene group which may contain a heteroatom. Specific examples of the C₁-C₄₀ hydrocarbylene group which may contain a heteroatom are as exemplified above for the specific examples of the C₁-C₄₀ hydrocarbylene group which may contain a heteroatom having X^{L1}, but not limited thereto.

As the sulfonium cation having formula (Z-3), a sulfonium cation having the formula (Z-3-1) is preferred:

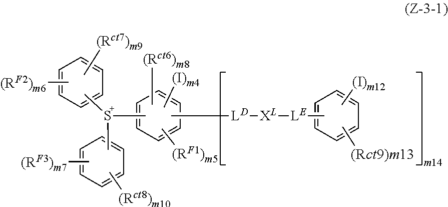

wherein m4 to m10, m12 to m14, R^{F1} to R^{F3}, R^{ct6} to R^{ct9}, L^{D}, L^{E}, and X^{L} are as defined above.

As the cation having the formula (Z-3-1), a cation having the formula (Z-3-2) is preferred:

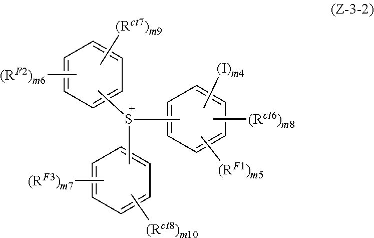

wherein m4 to m10, R^{F1} to R^{F3}, and R^{ct6} to R^{ct8} are as defined above.

Specific examples of the sulfonium cation having formula (Z-3) are shown below, but not limited thereto. Herein Me is a methyl group.

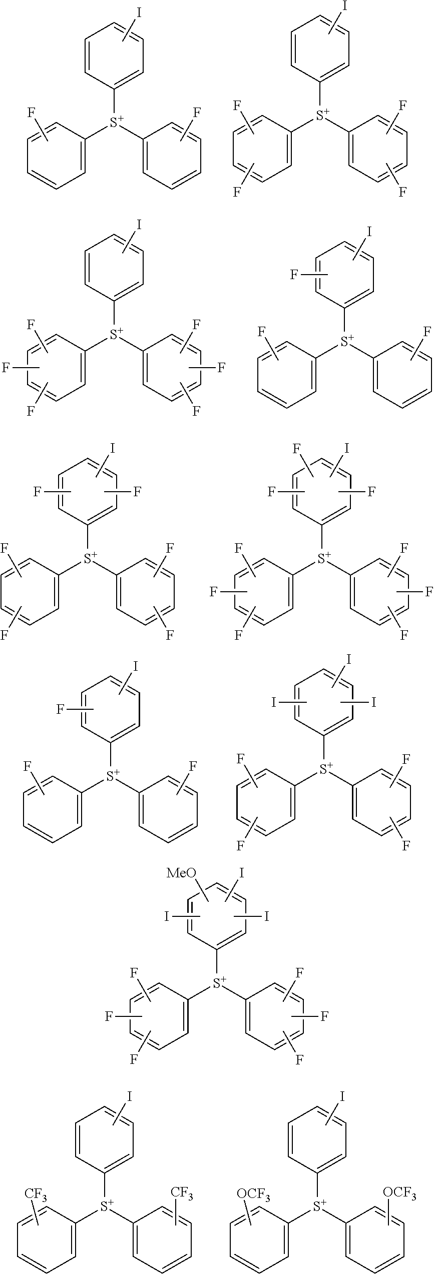

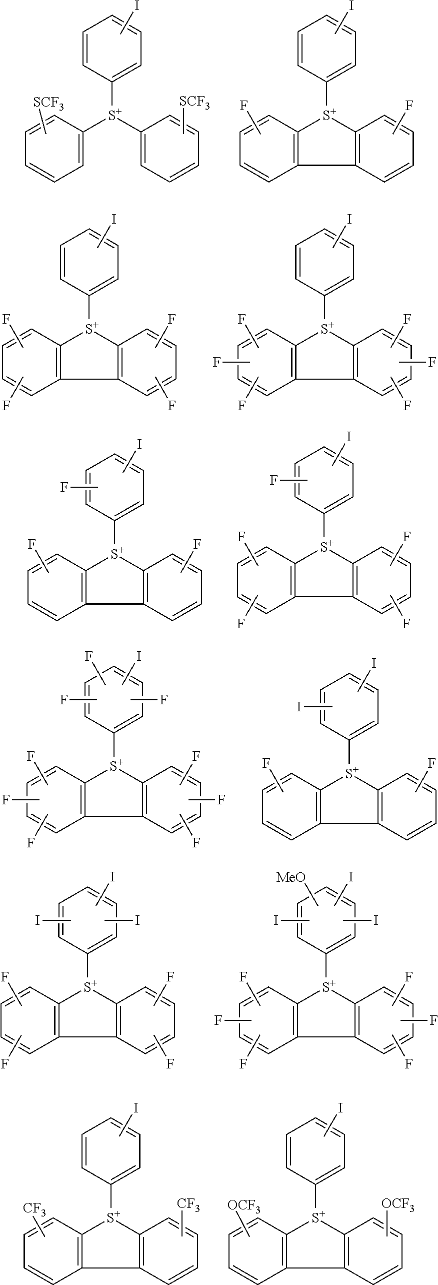

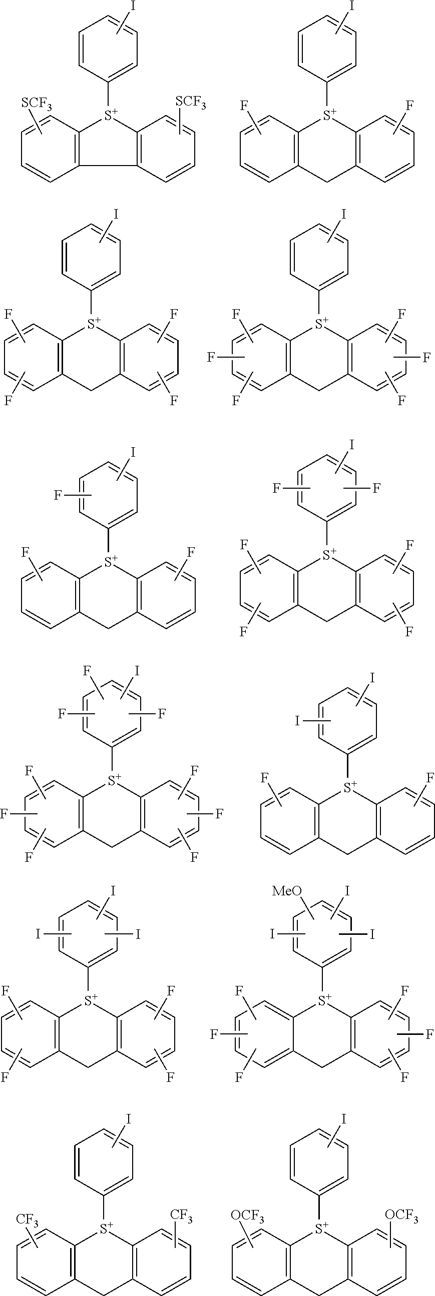

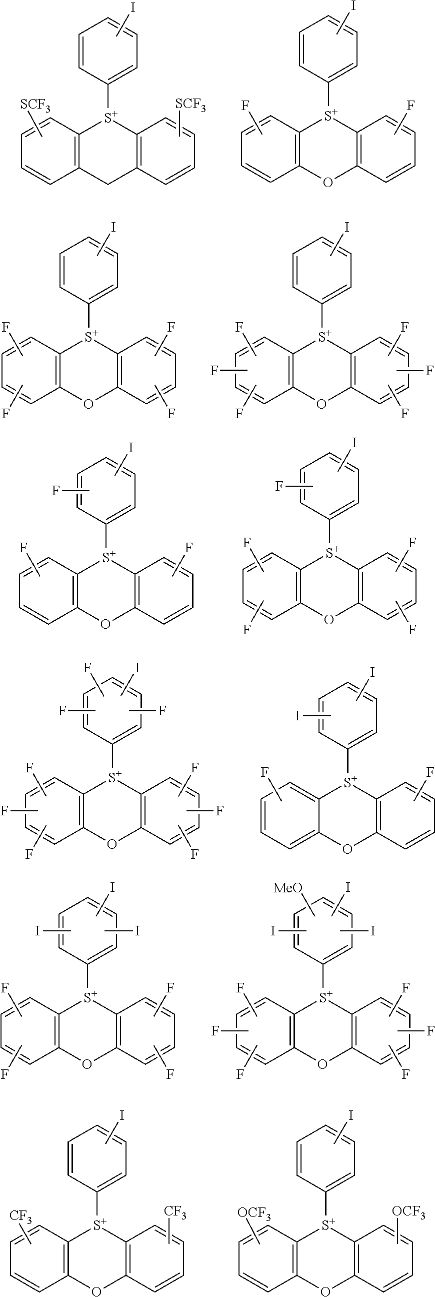

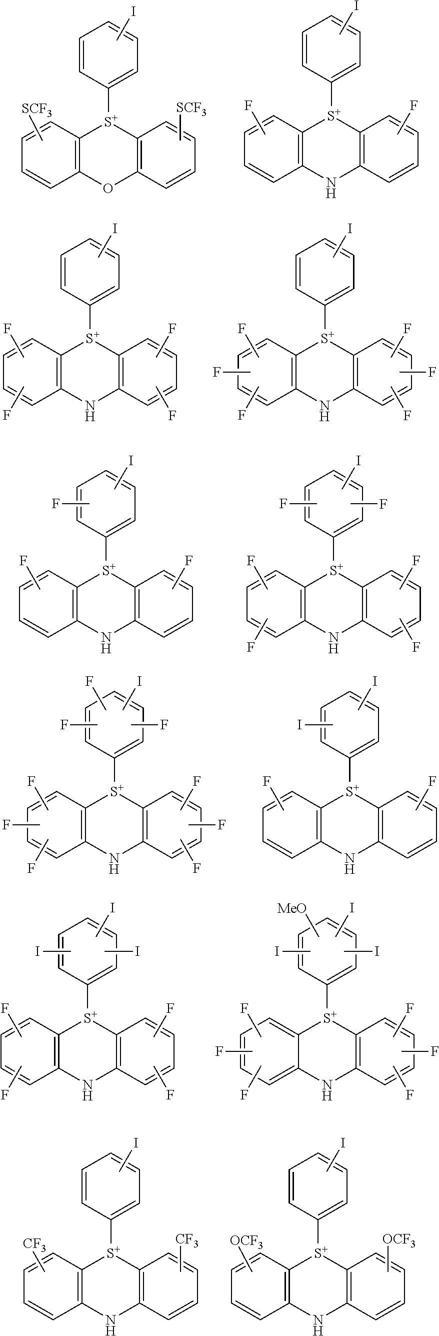

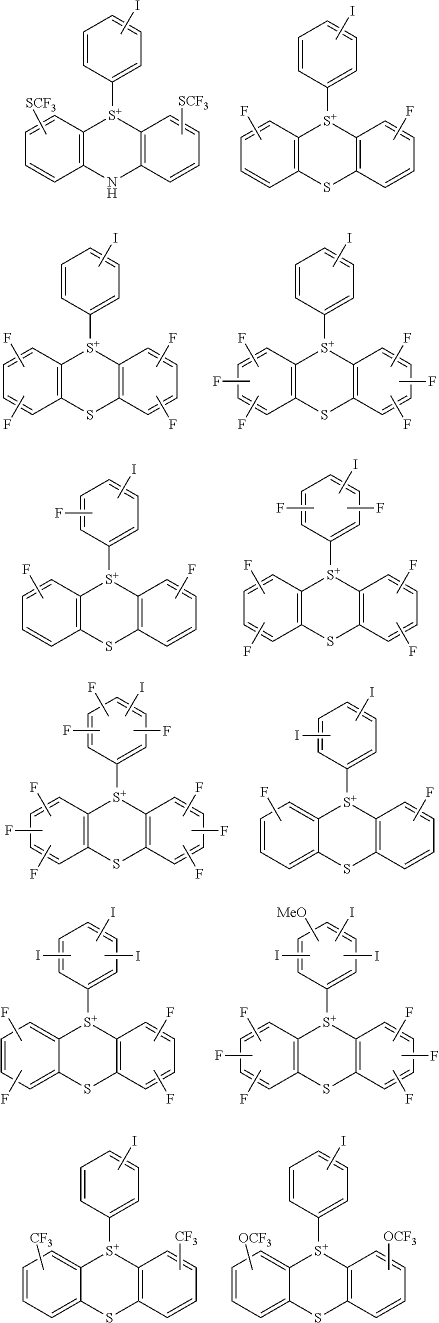

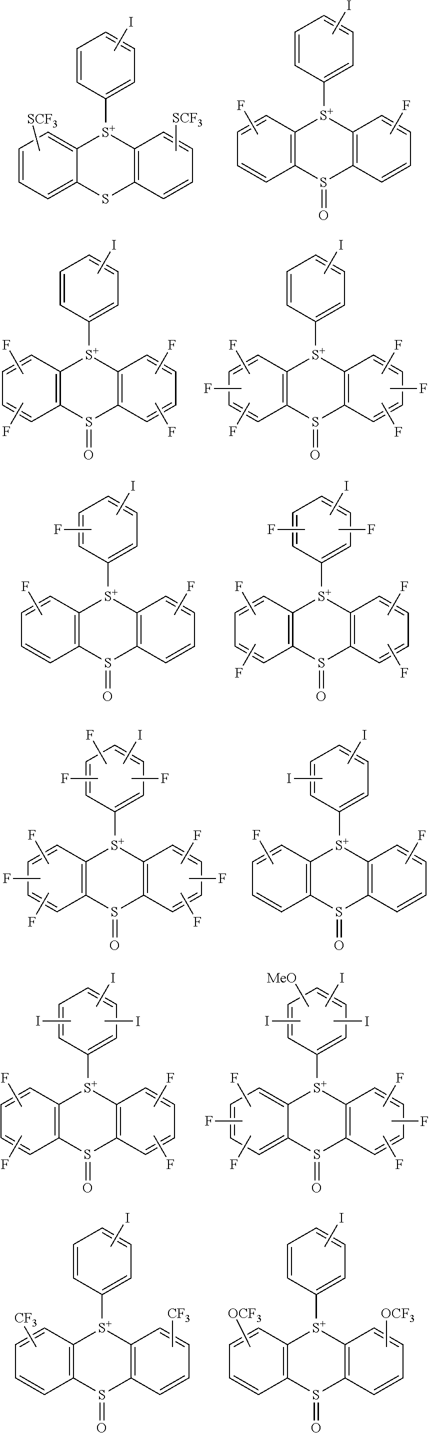

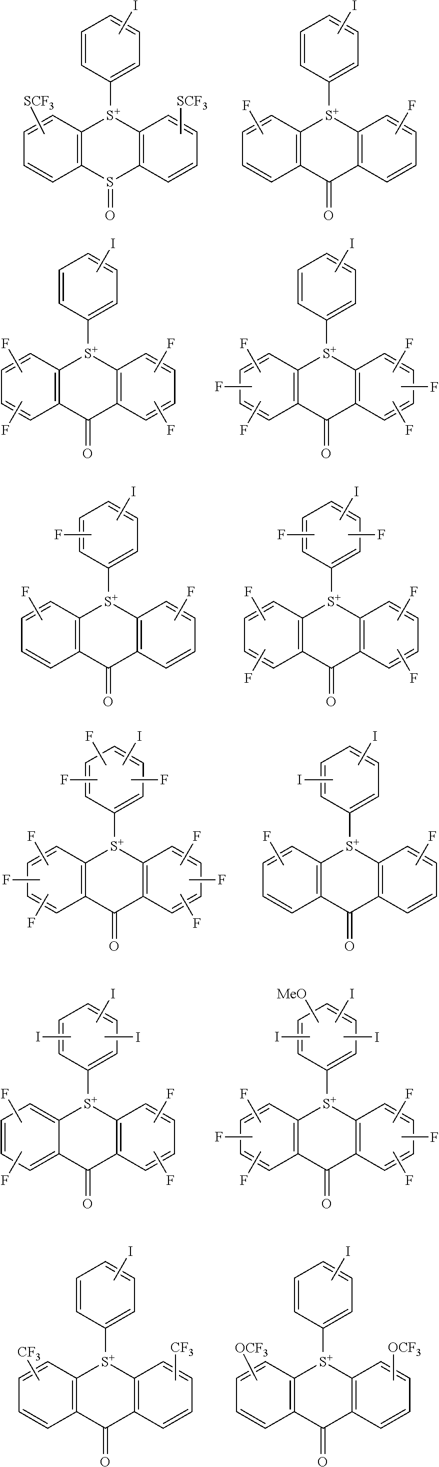

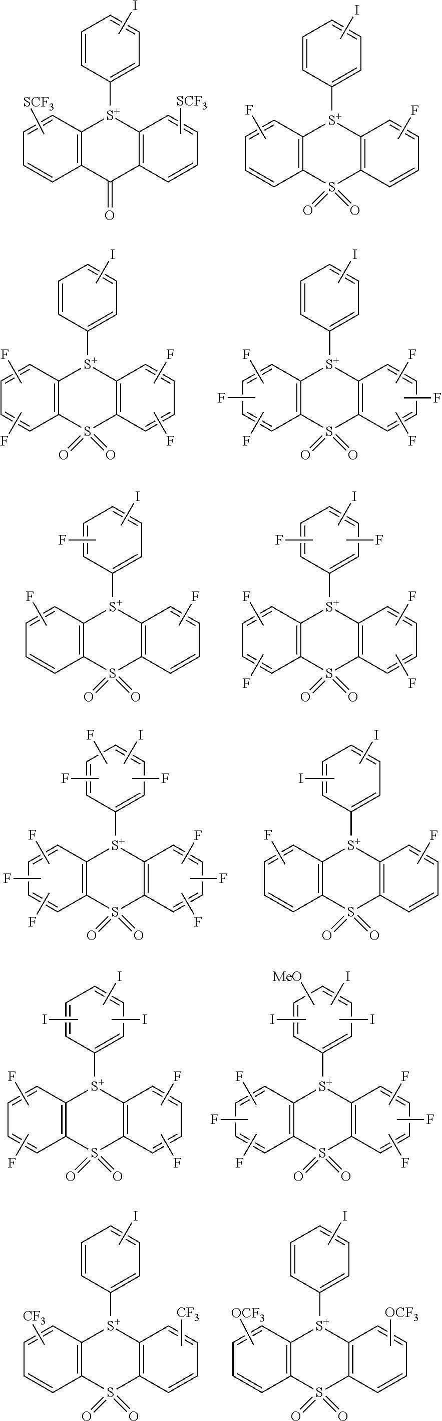

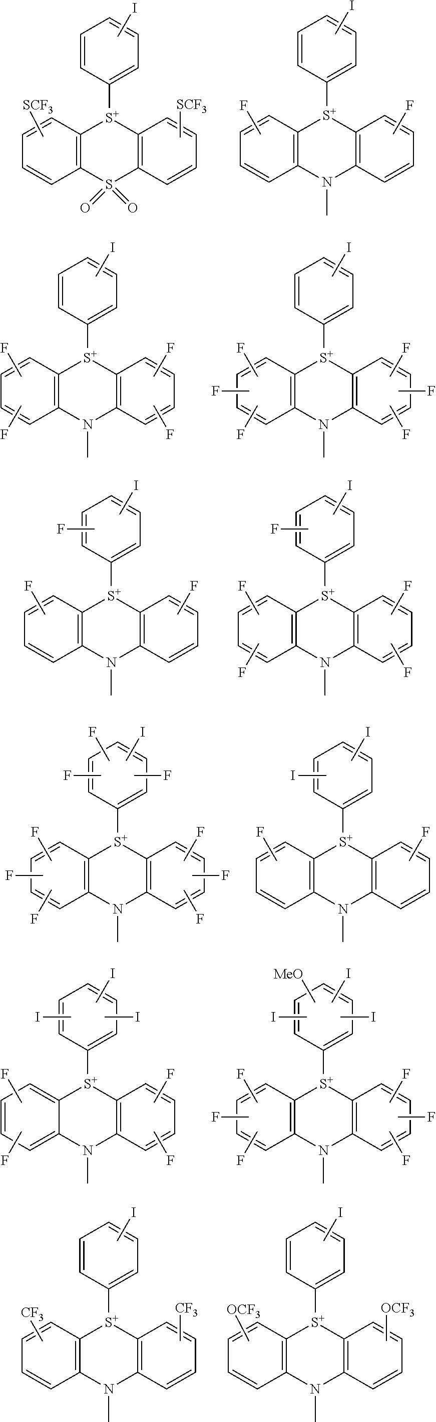

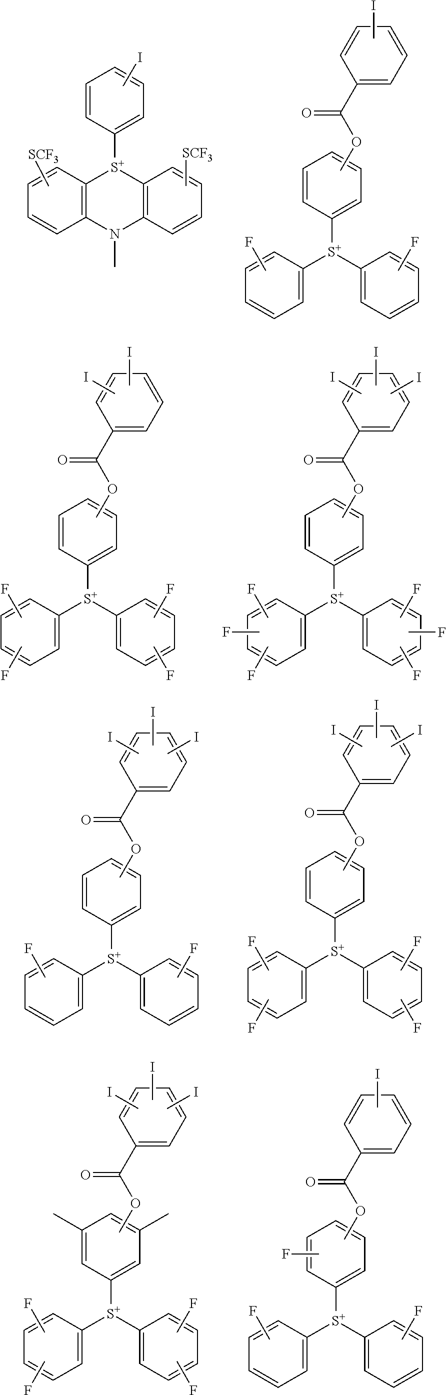

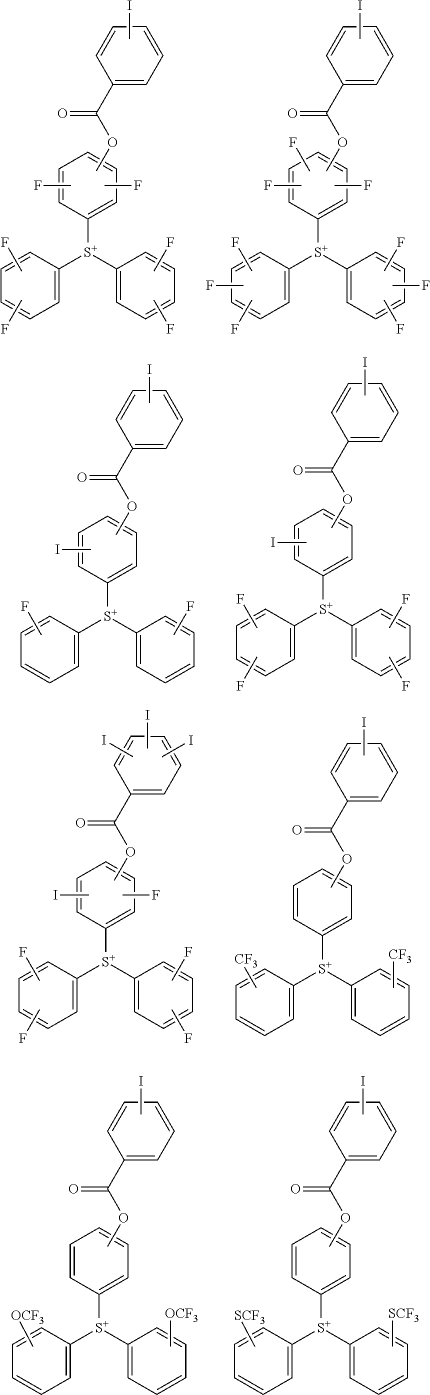

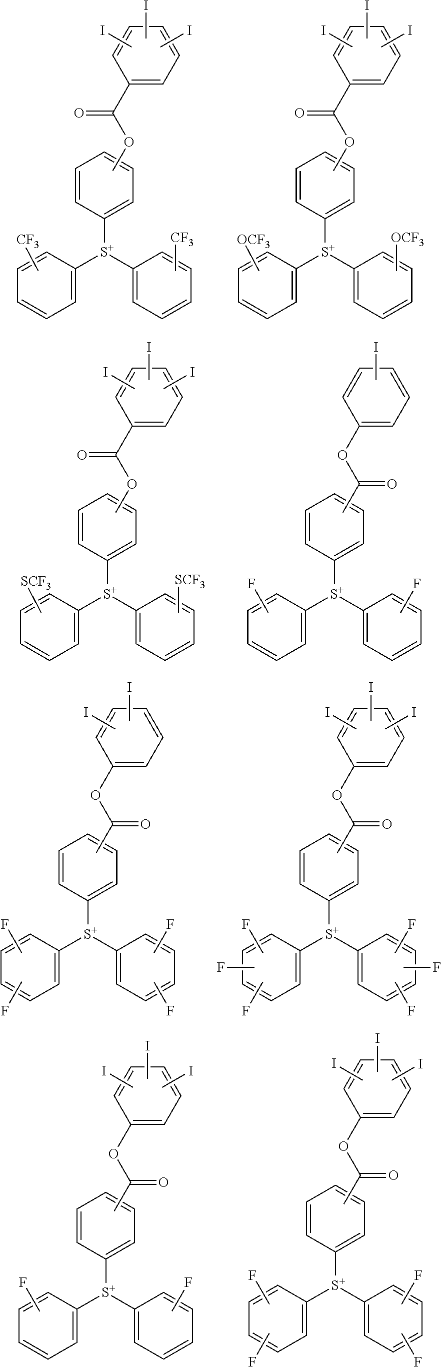

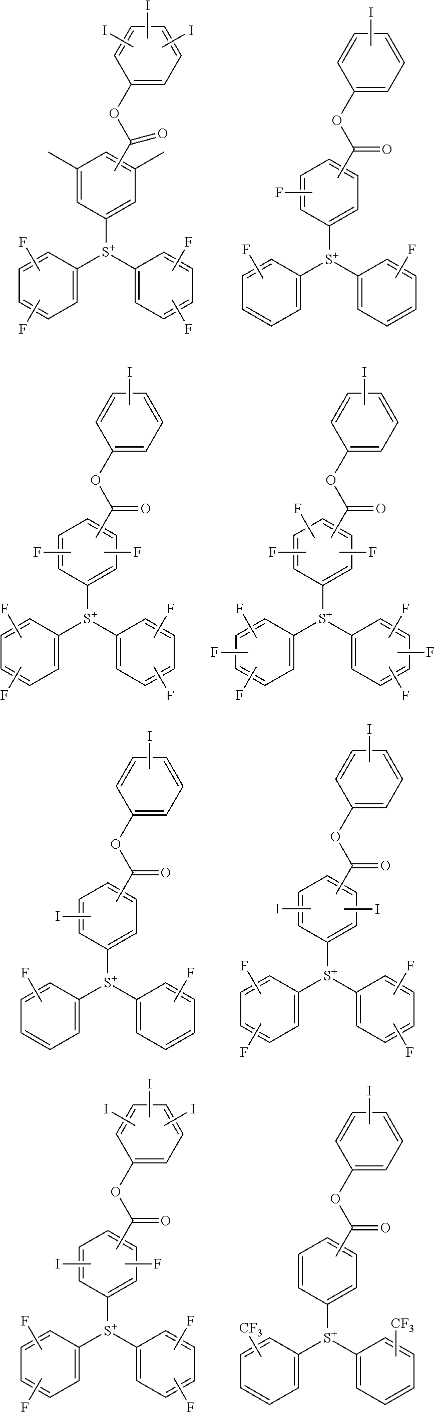

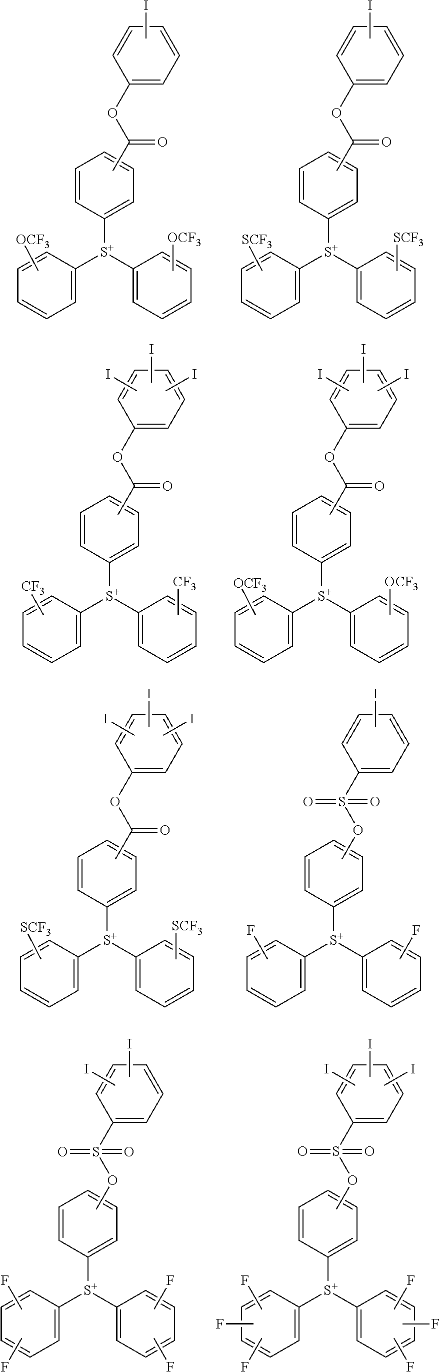

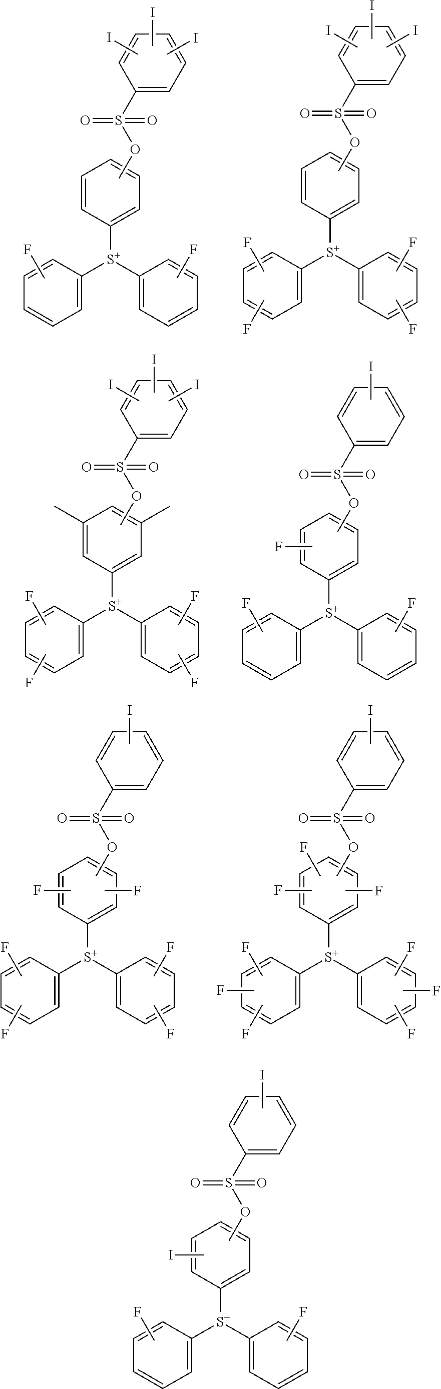

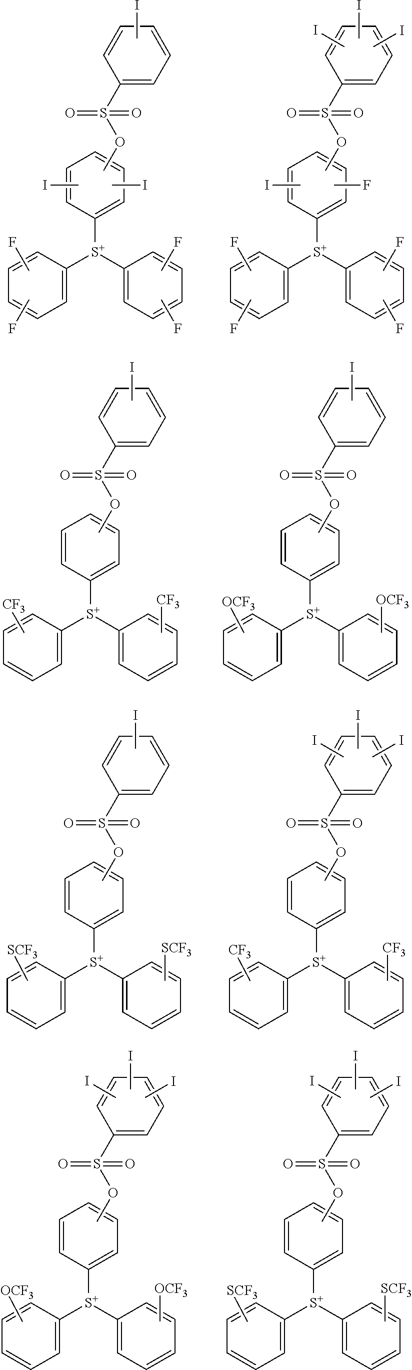

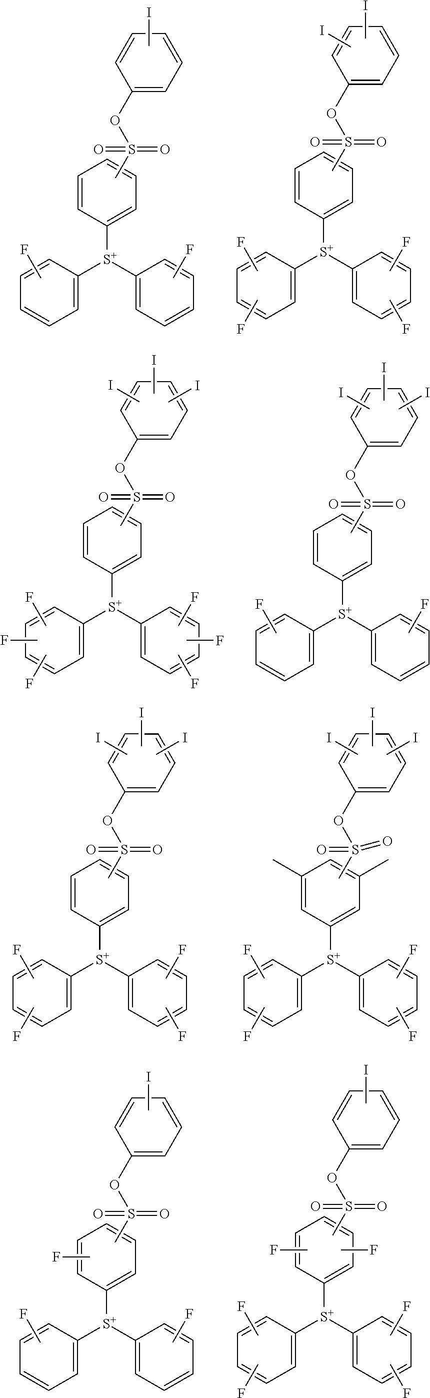

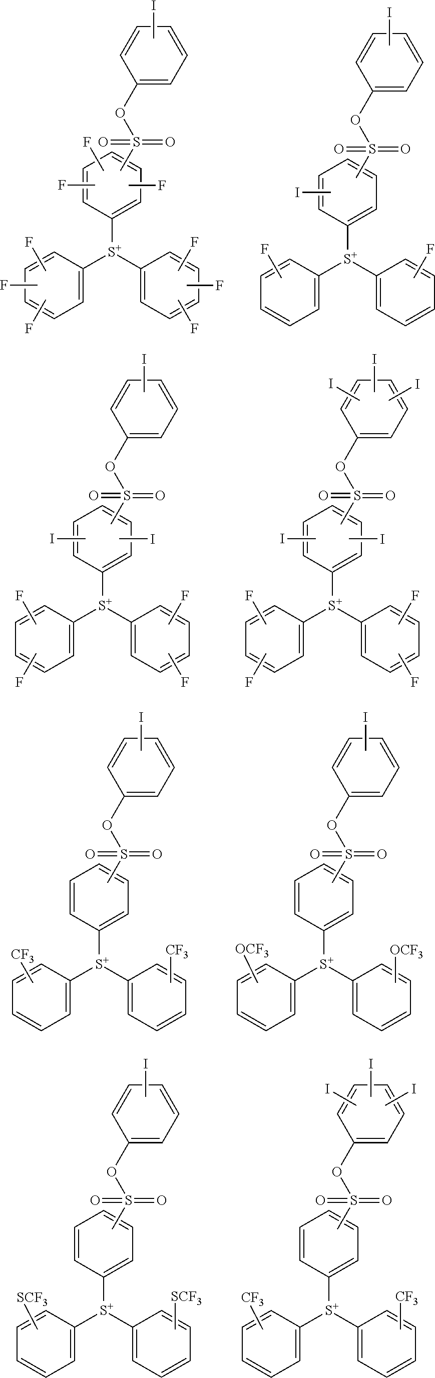

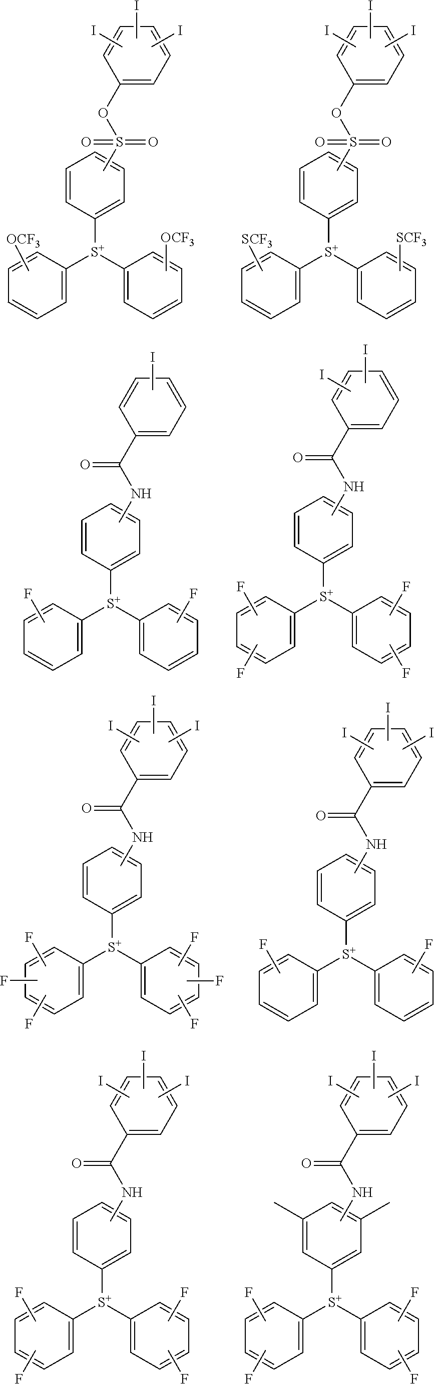

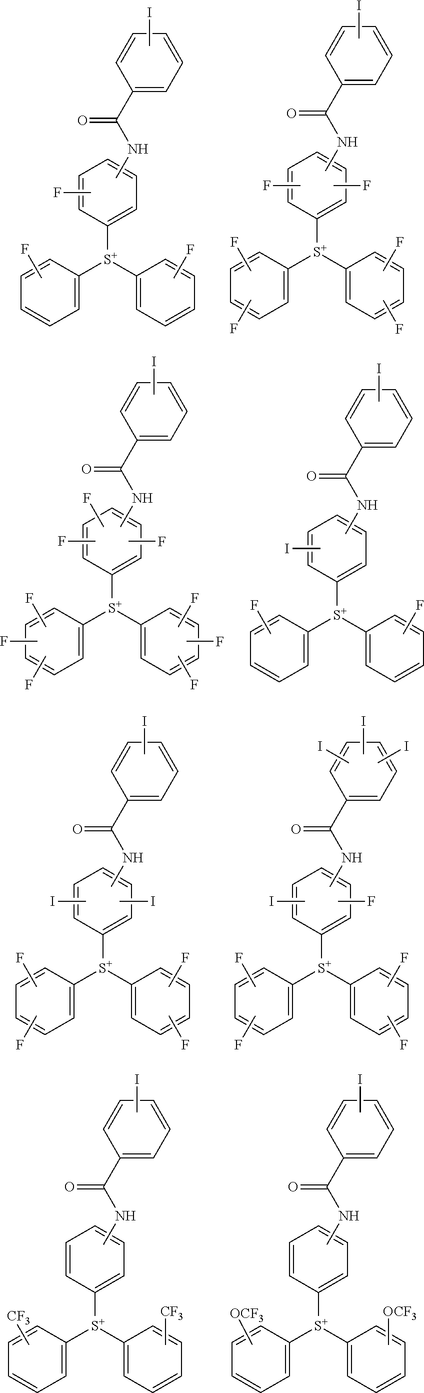

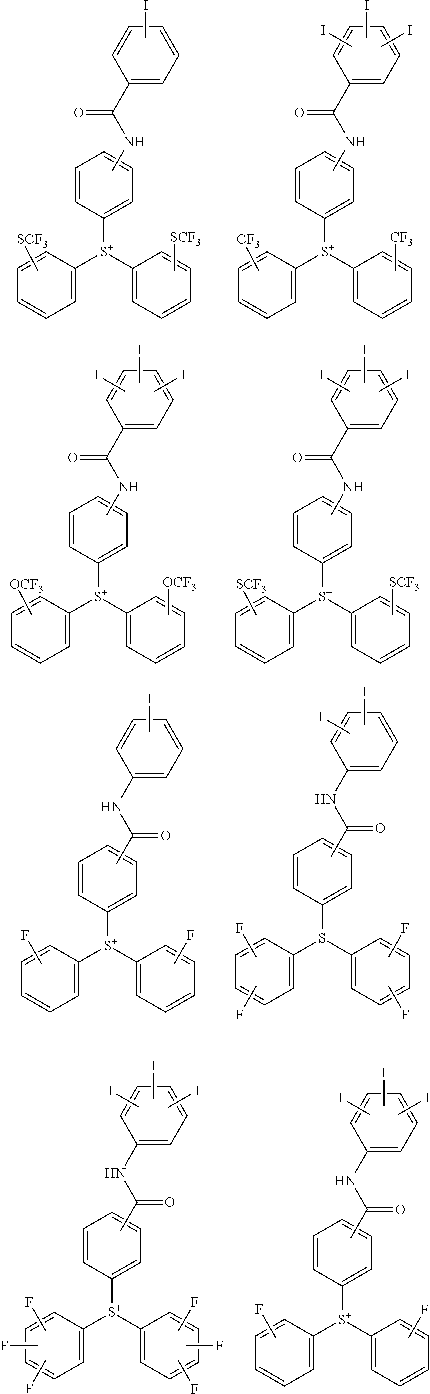

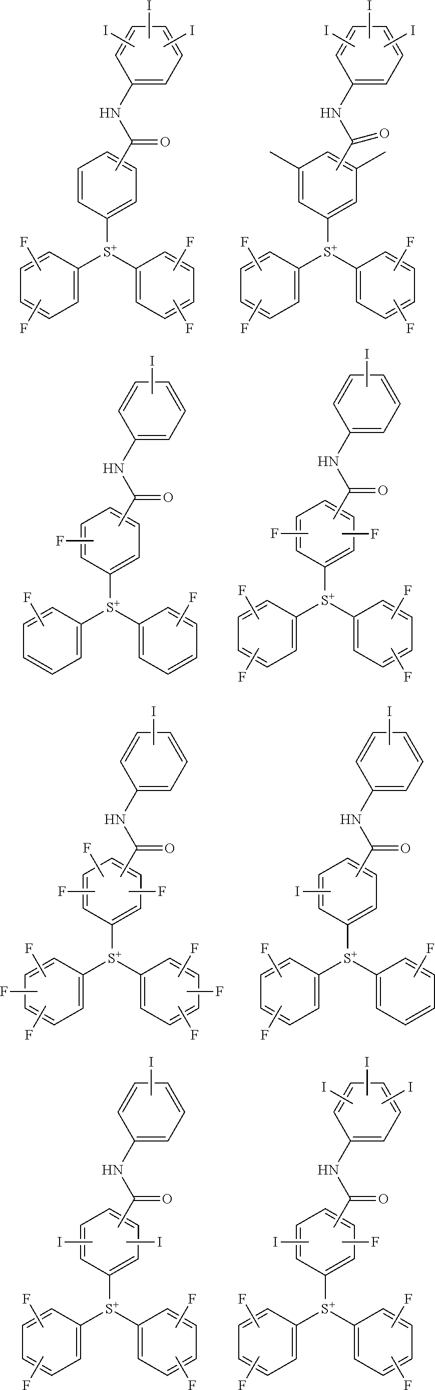

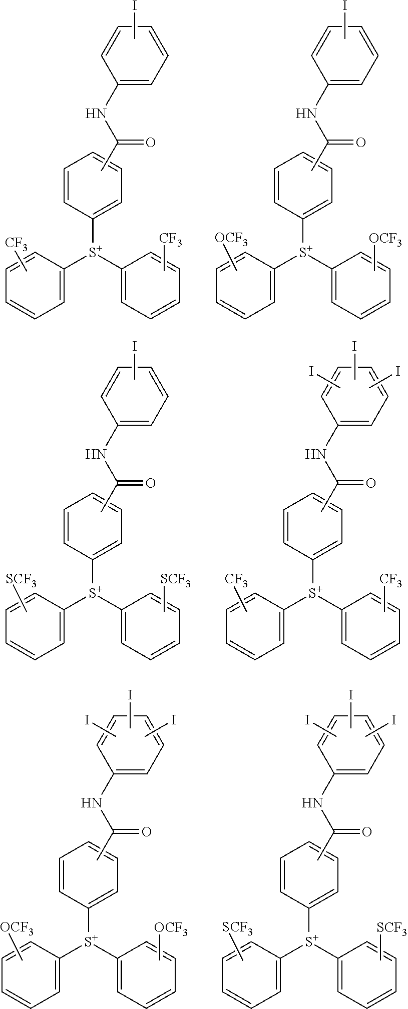

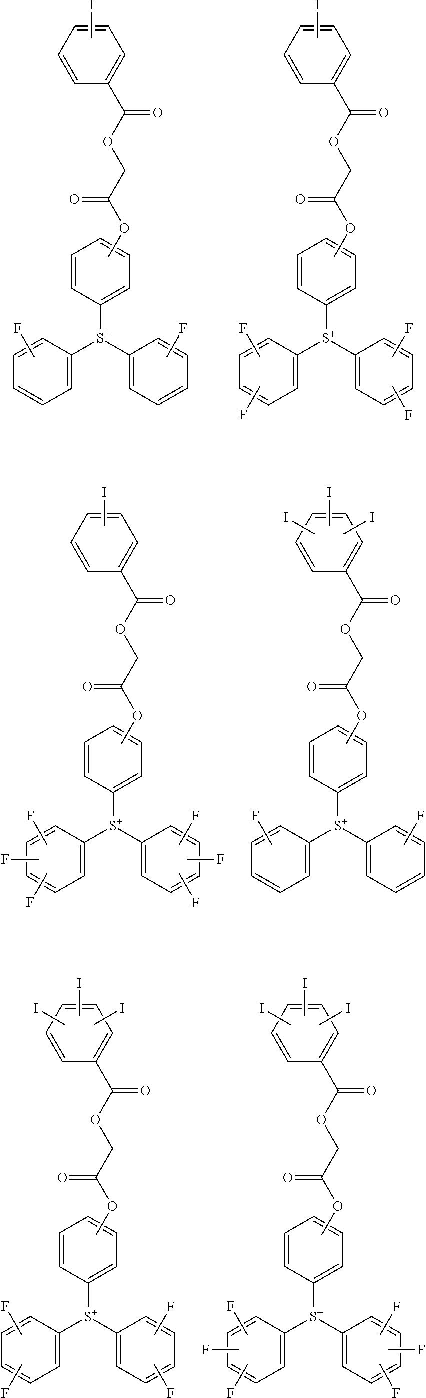

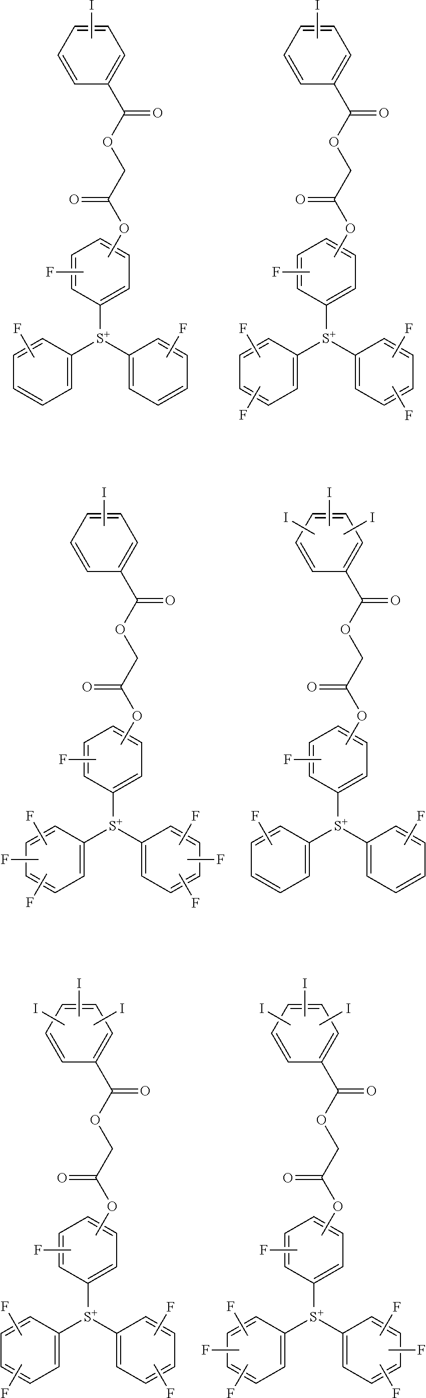

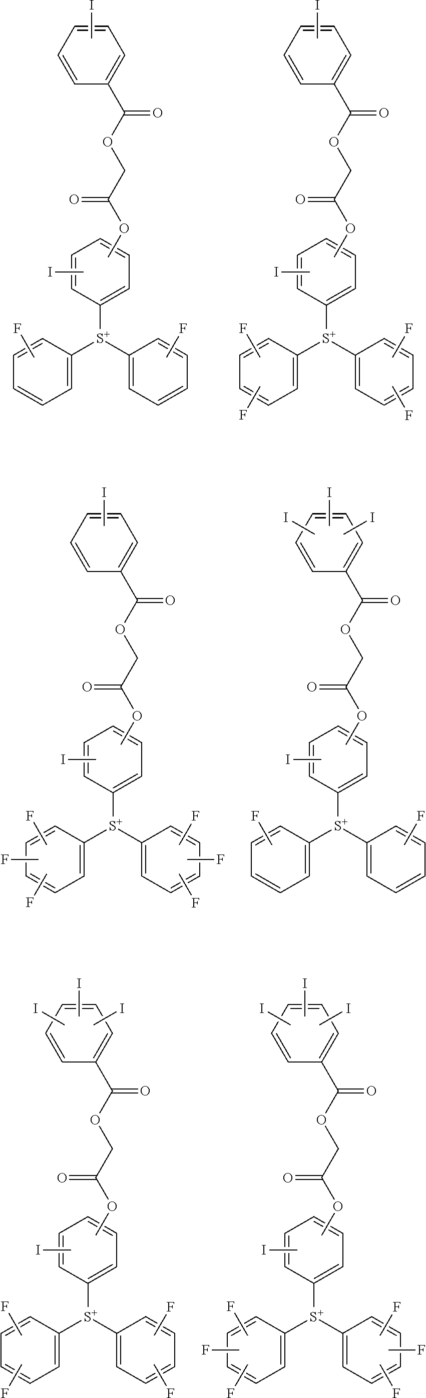

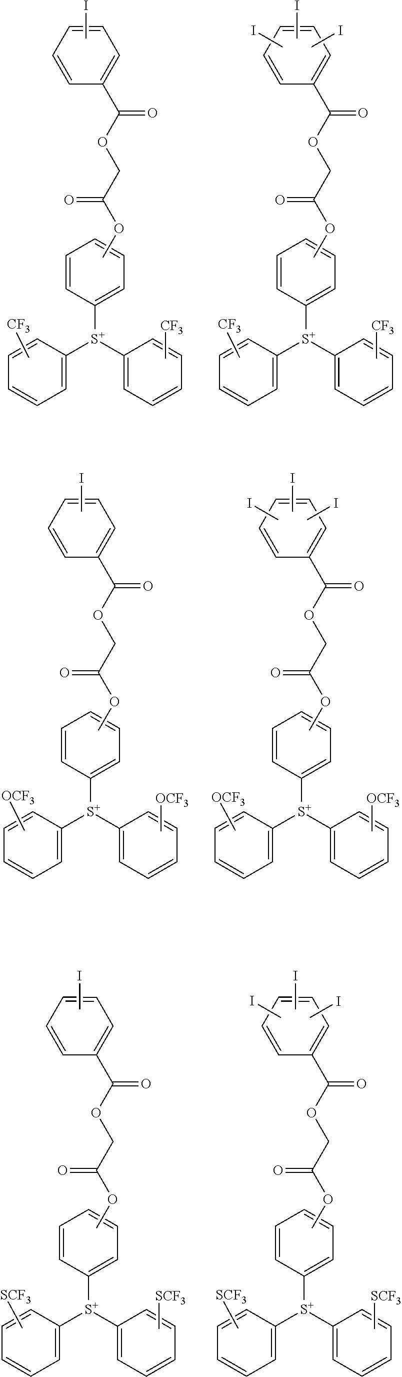

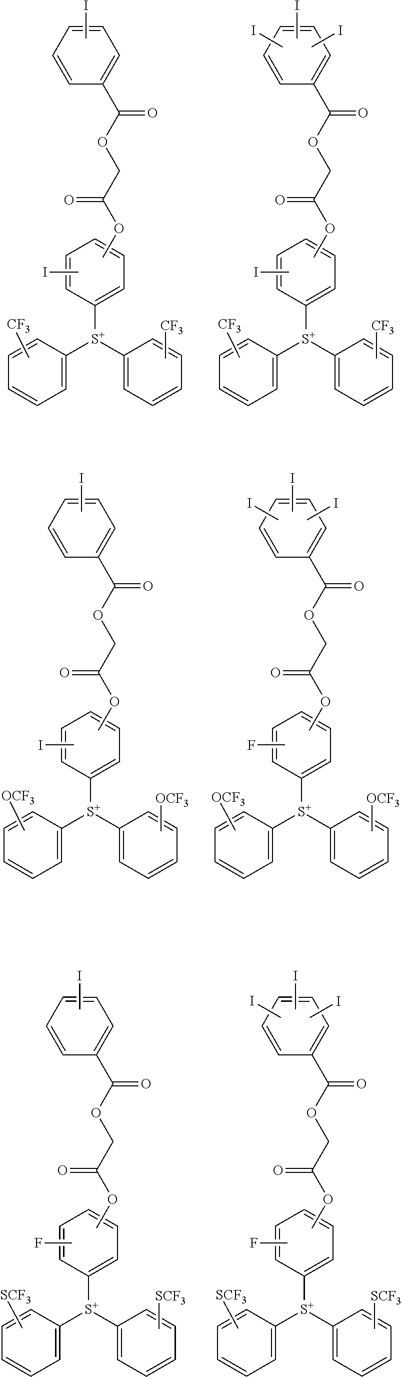

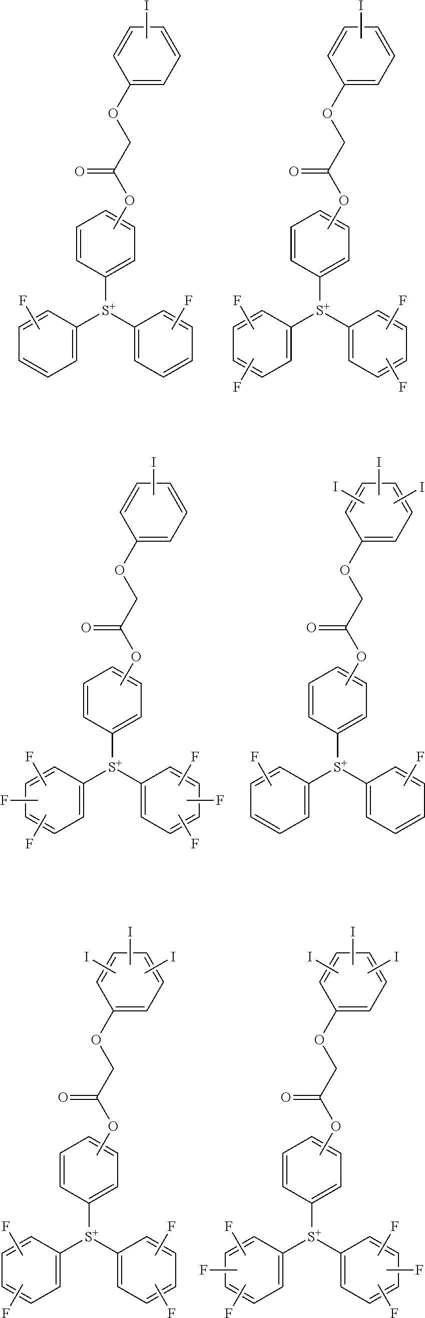

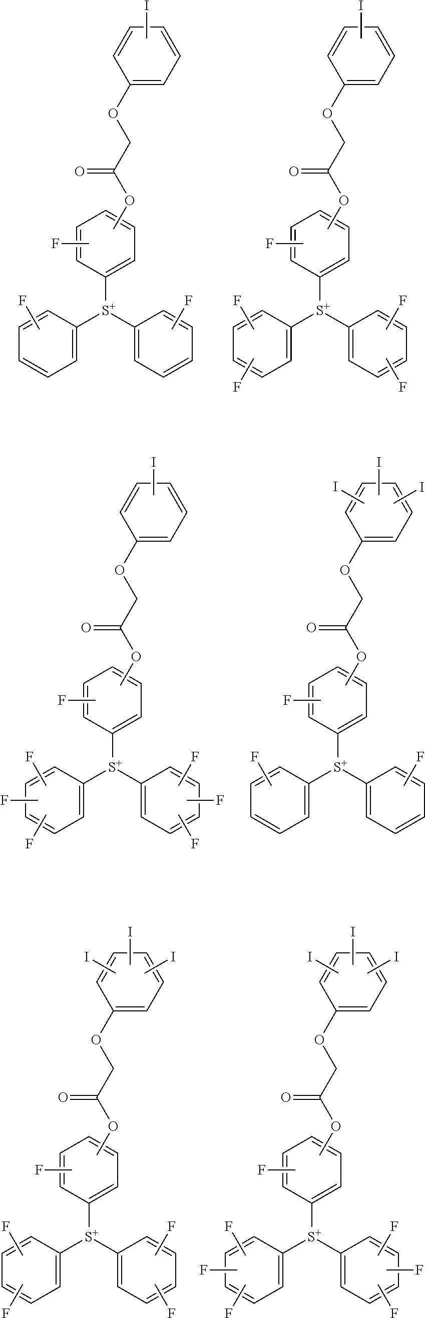

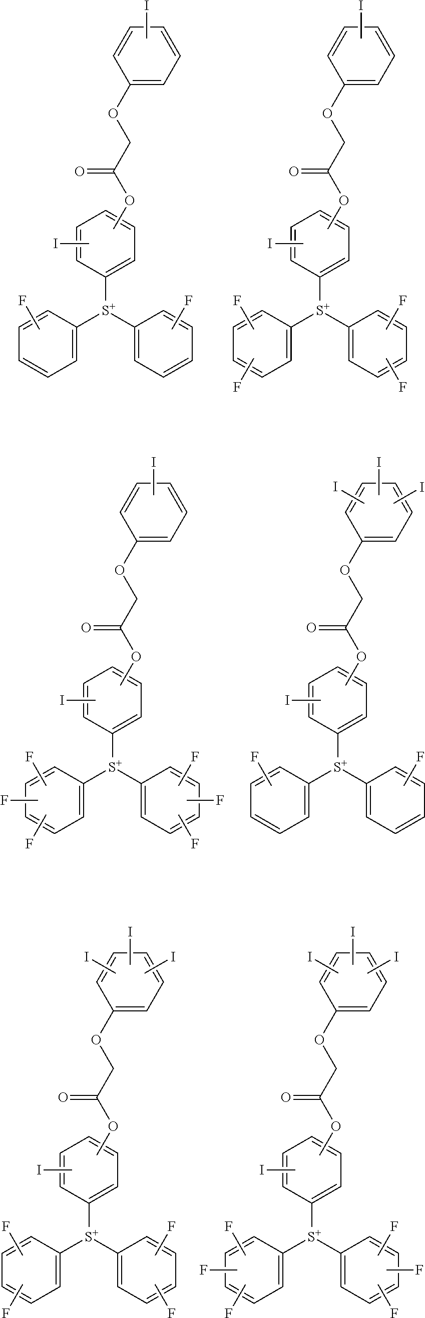

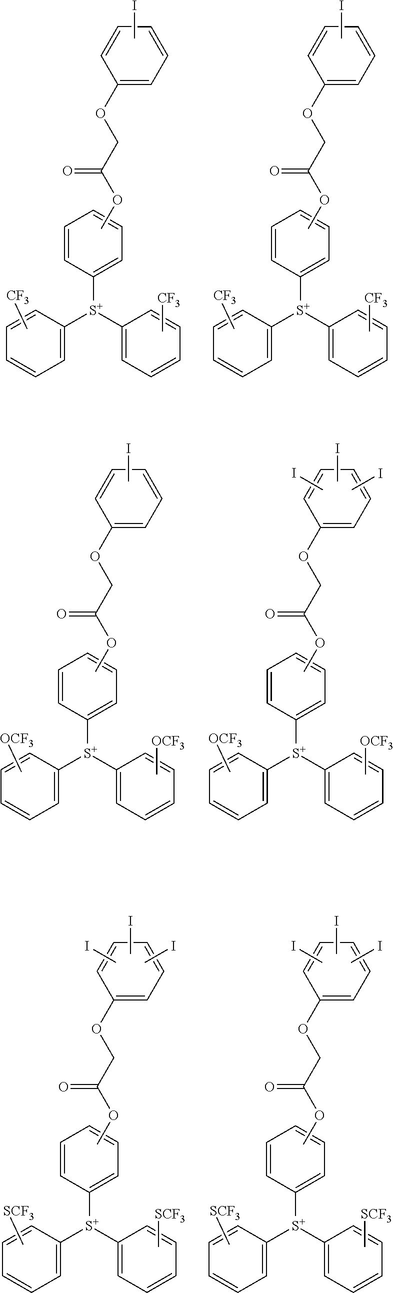

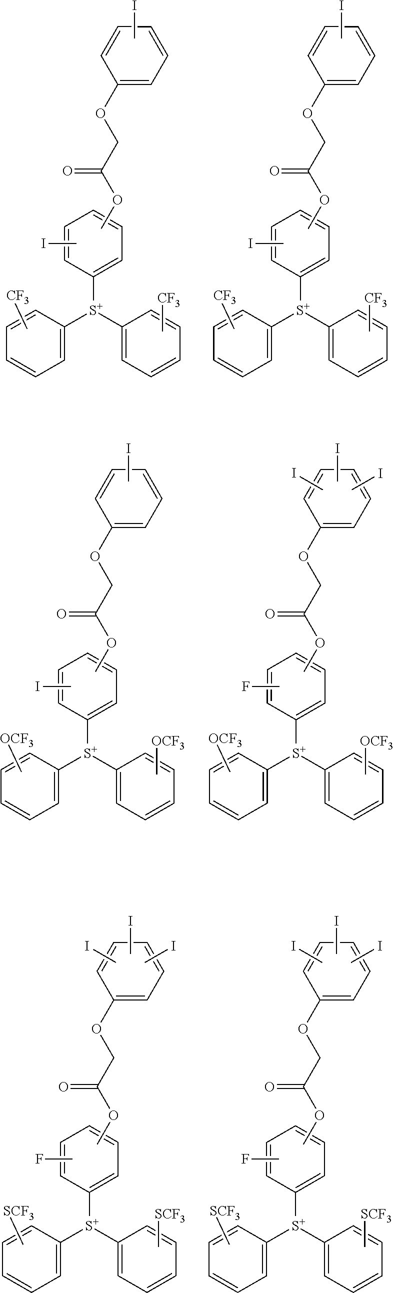

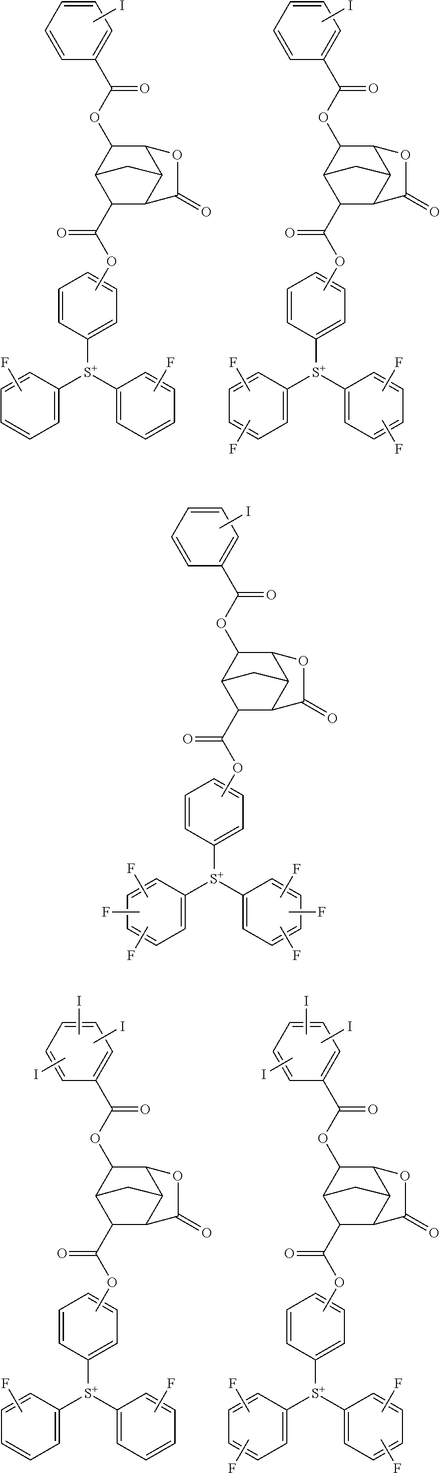

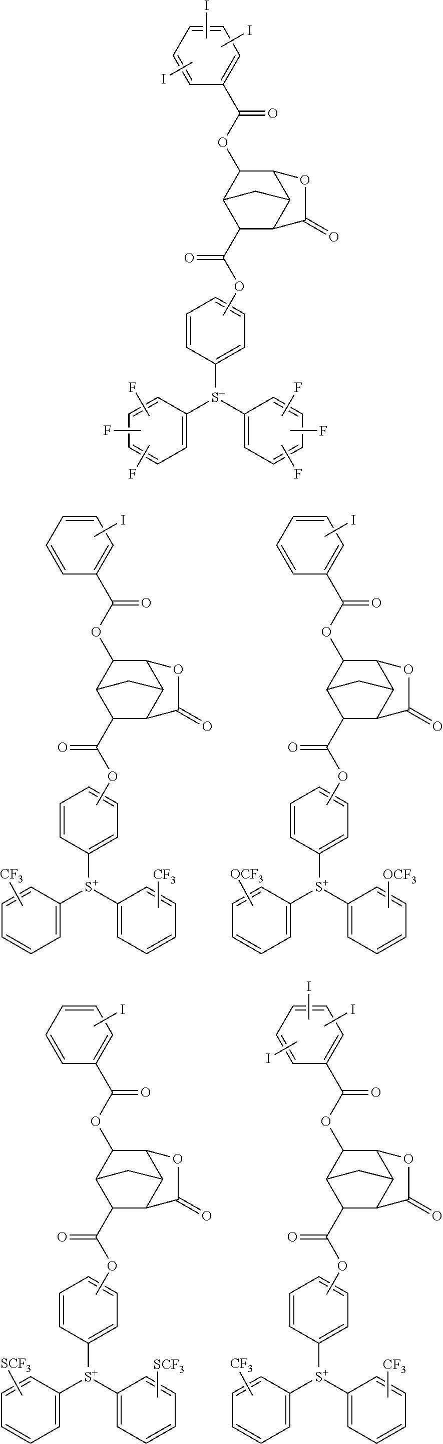

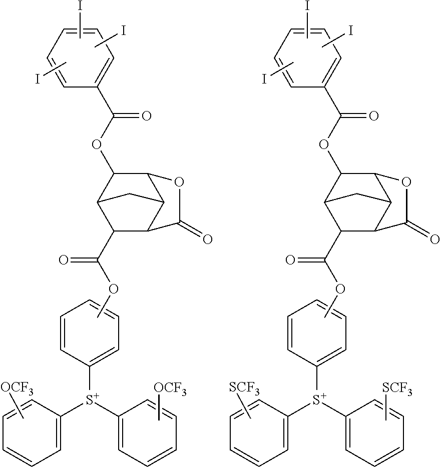

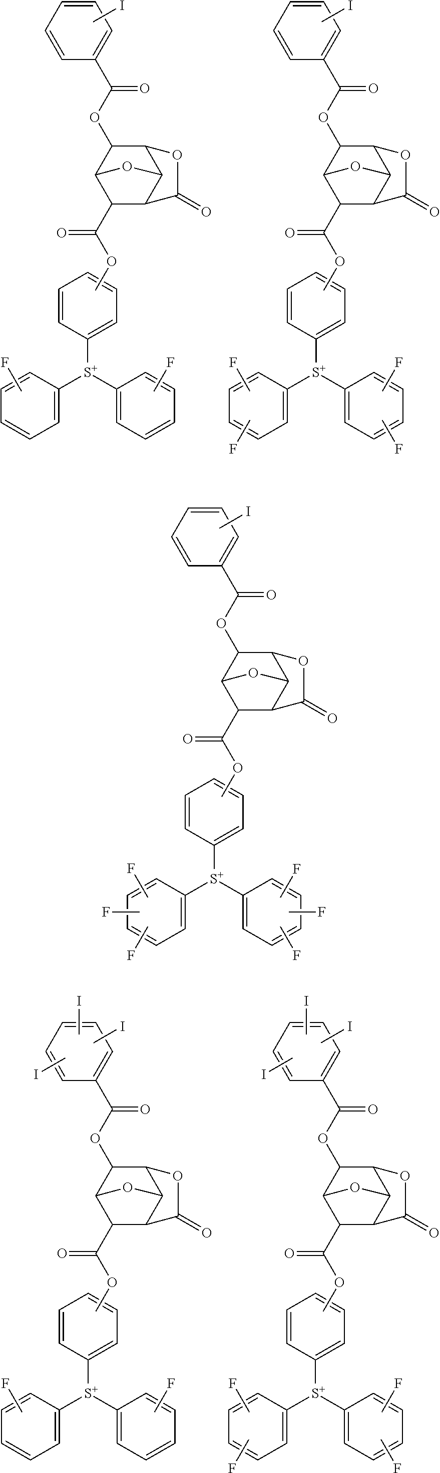

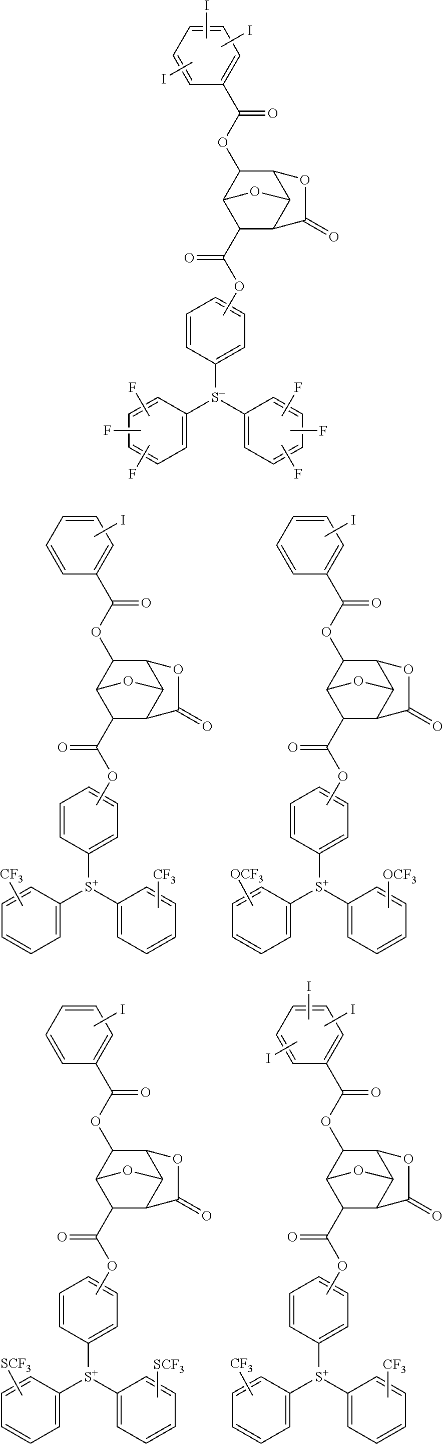

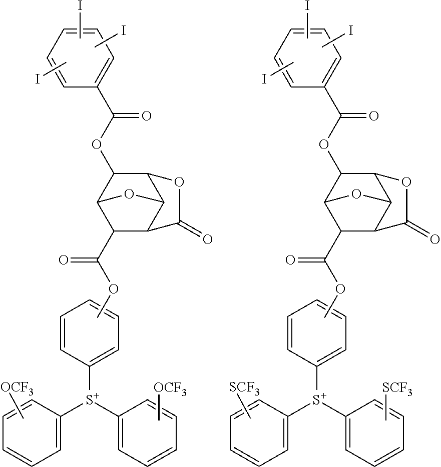

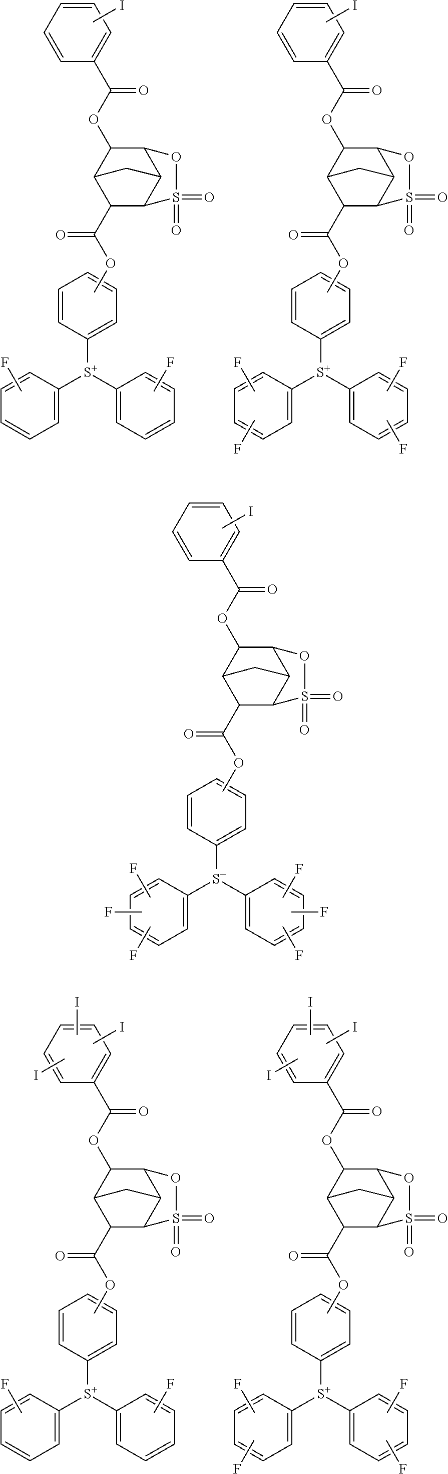

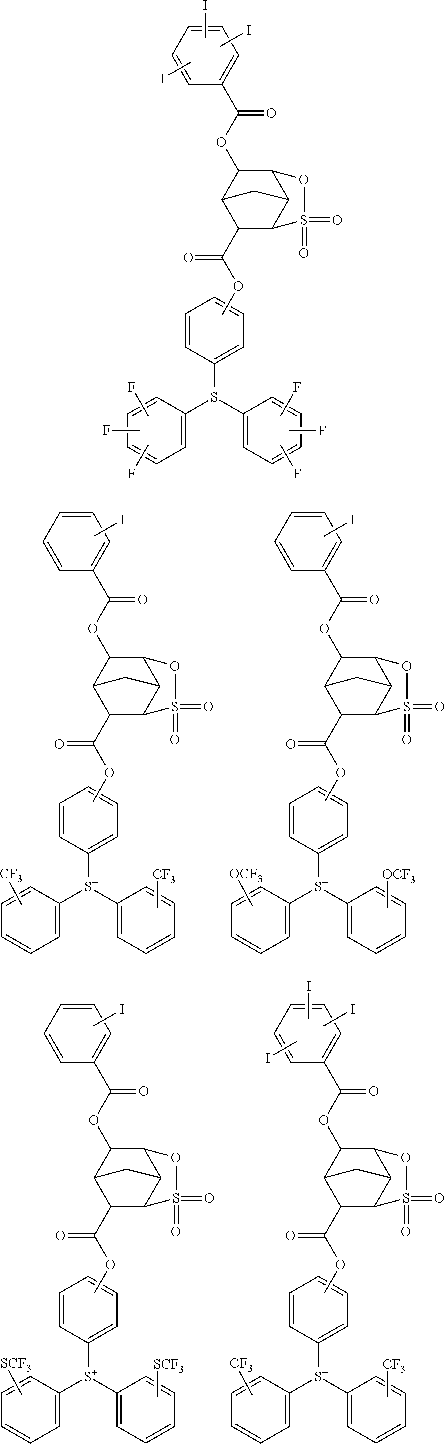

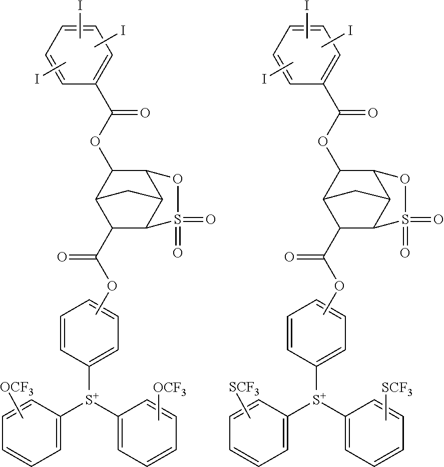

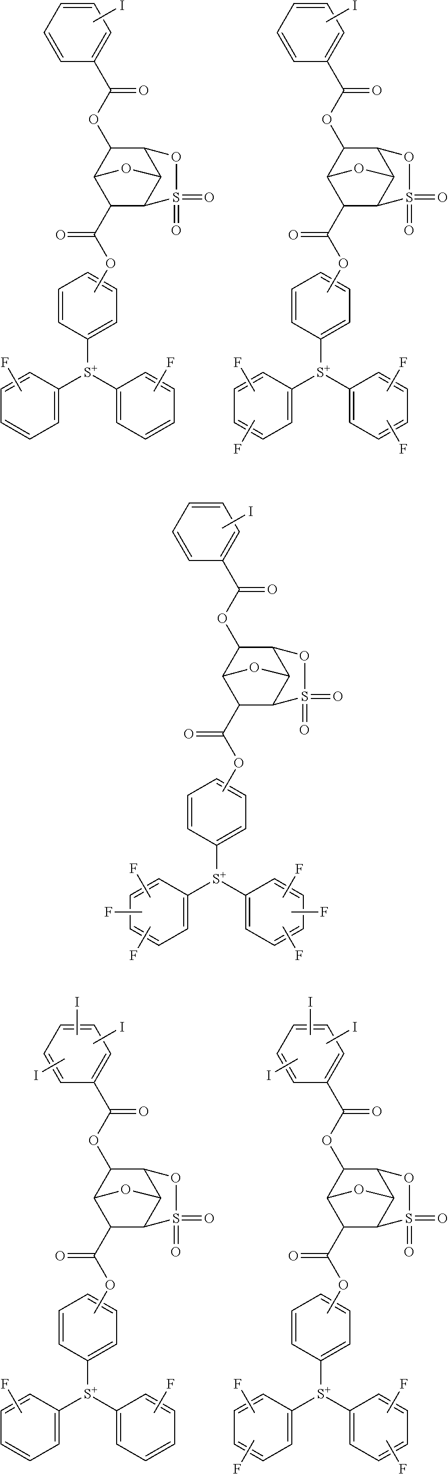

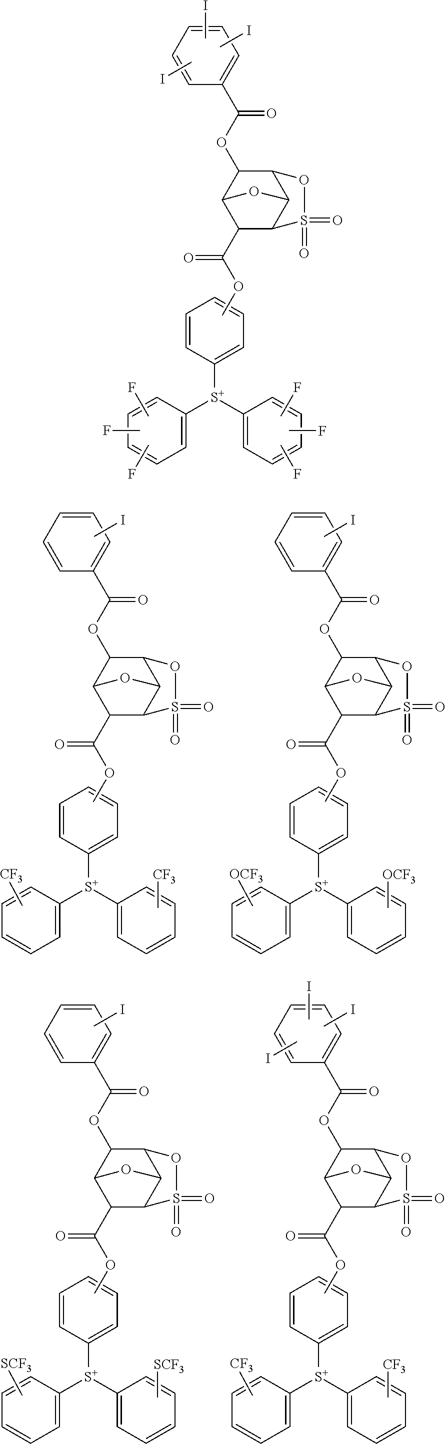

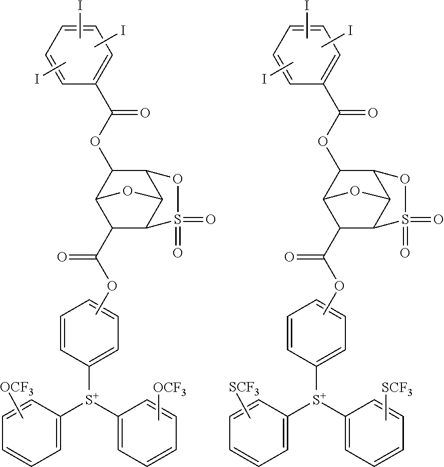

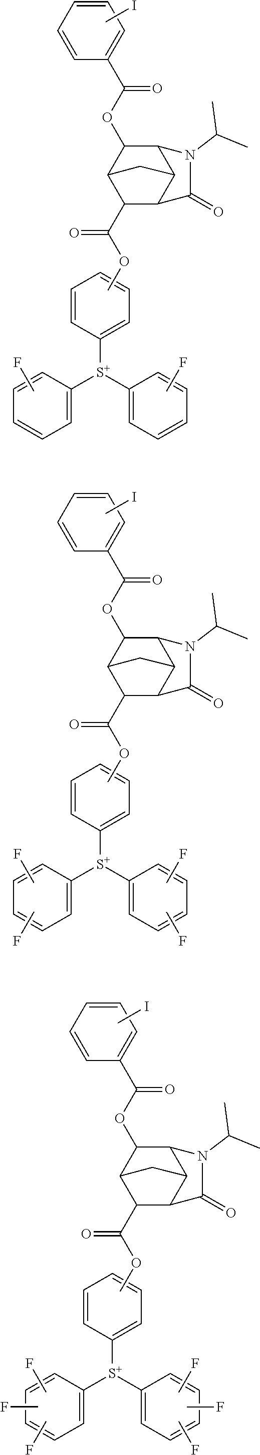

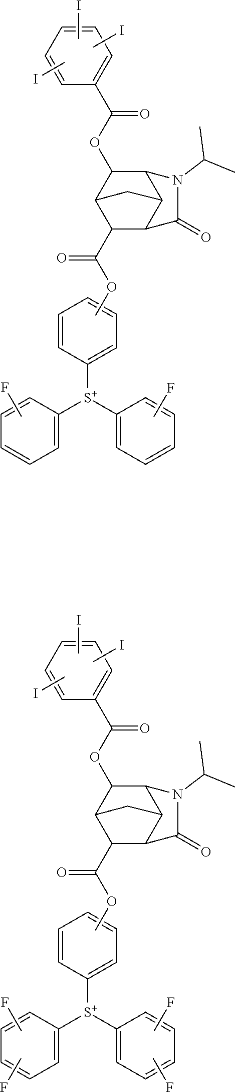

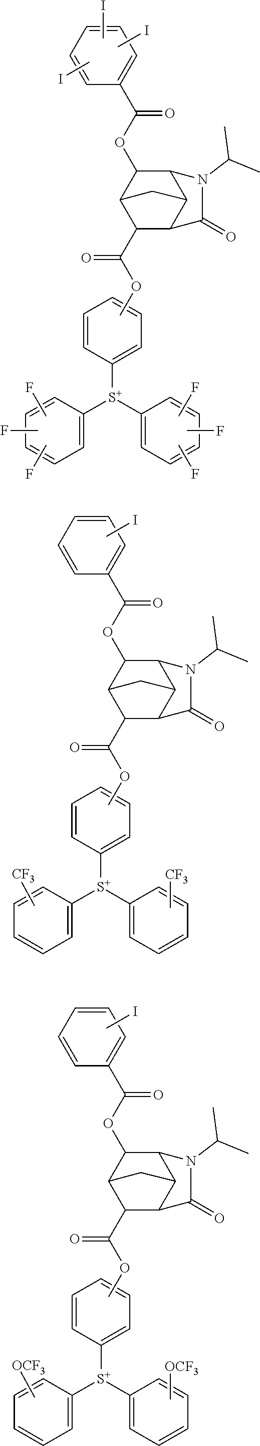

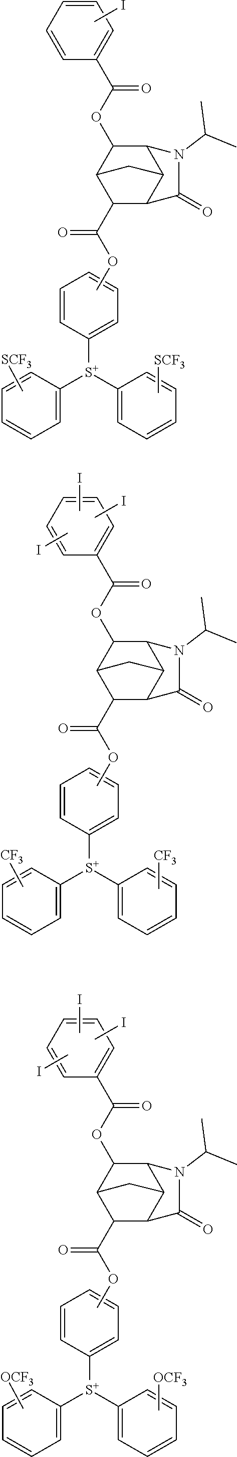

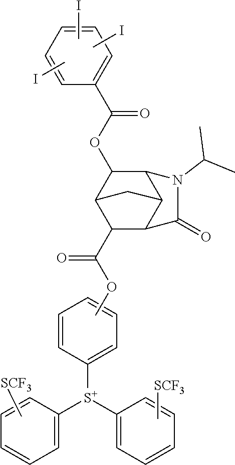

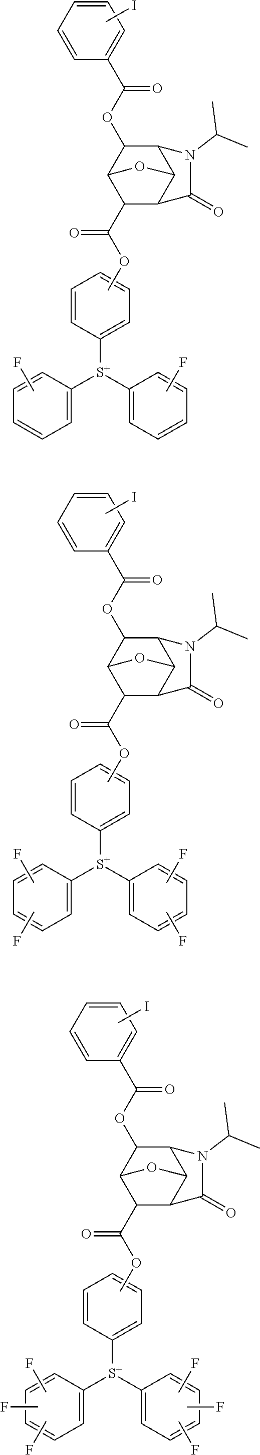

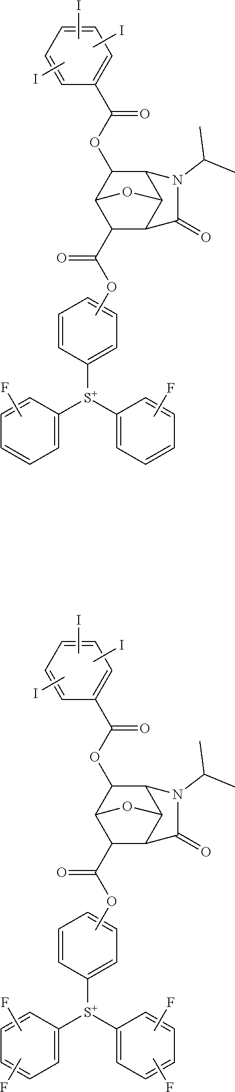

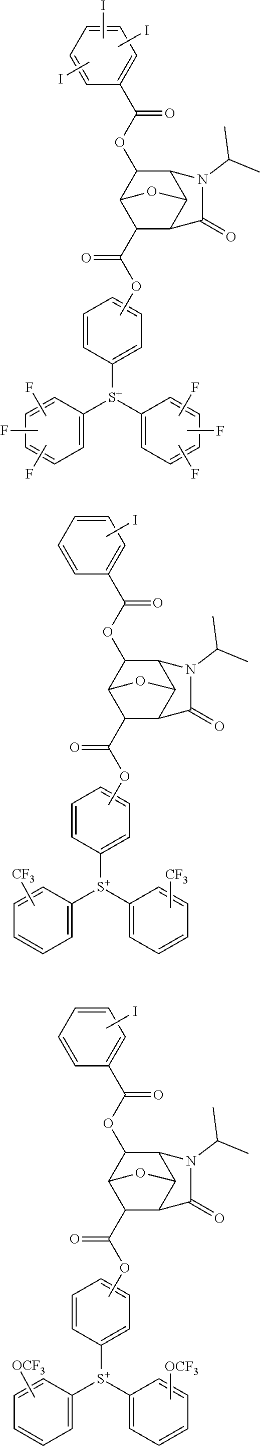

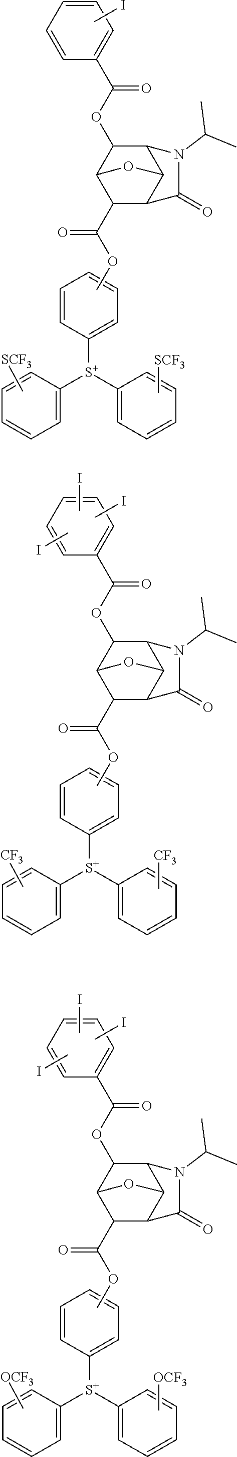

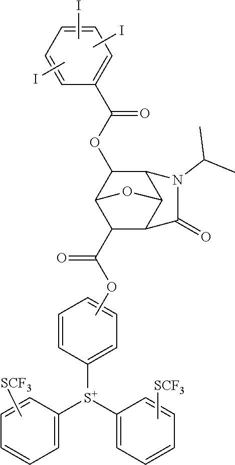

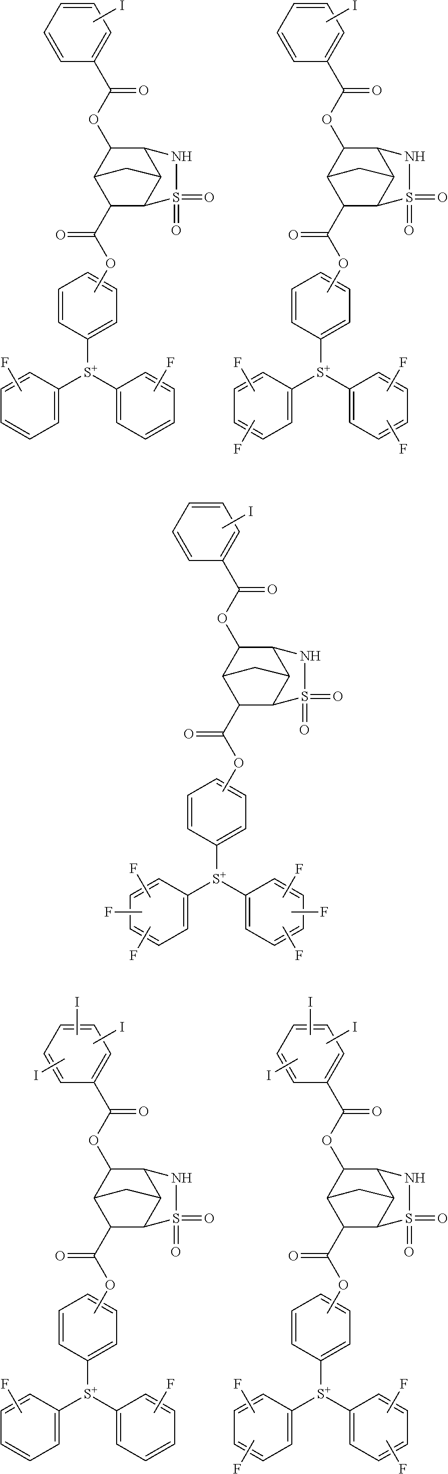

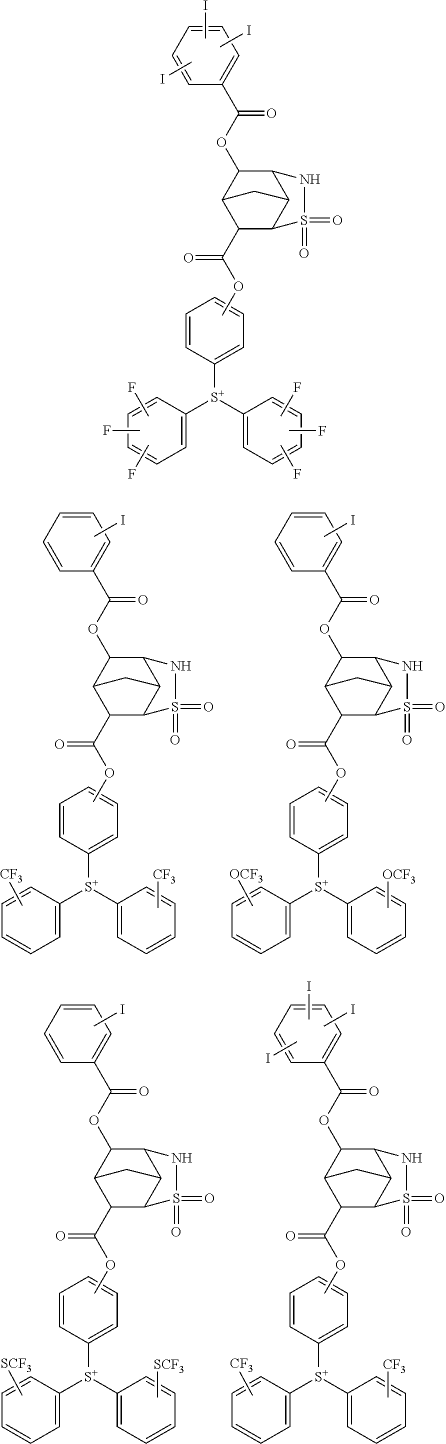

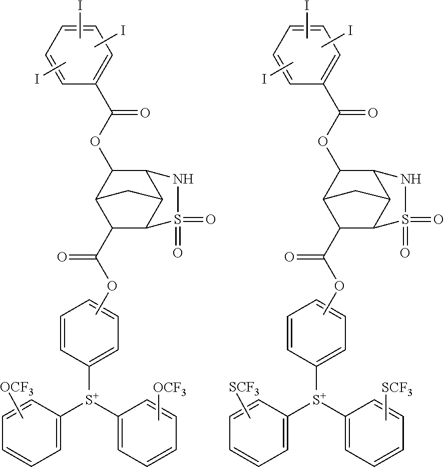

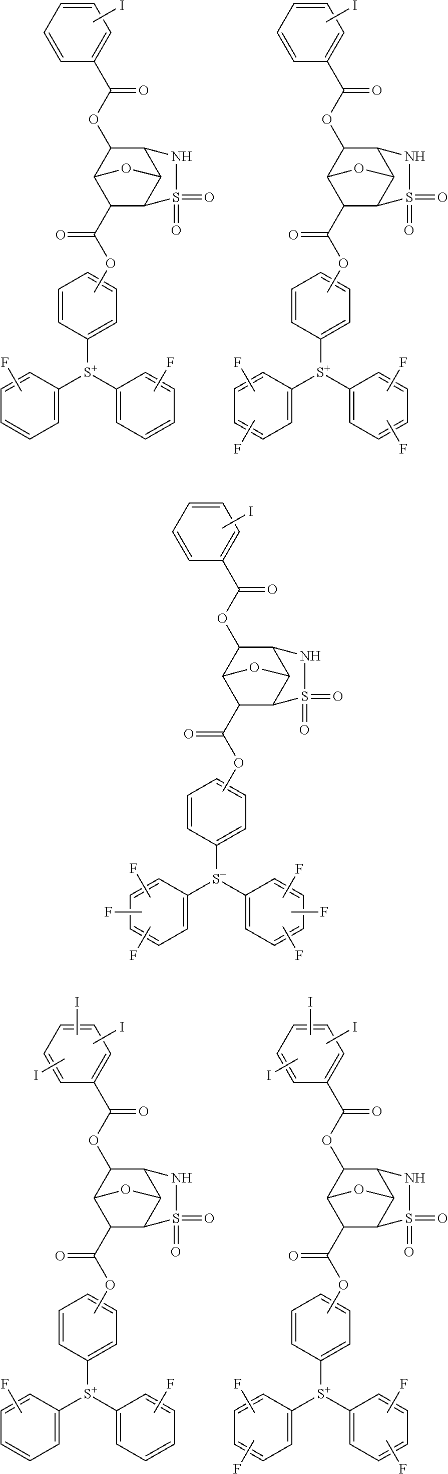

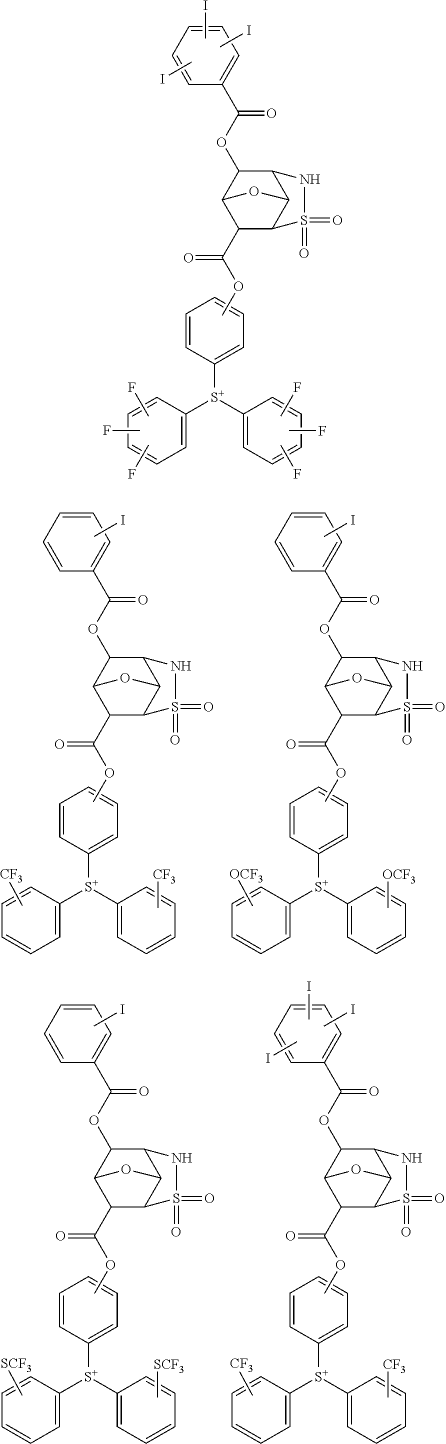

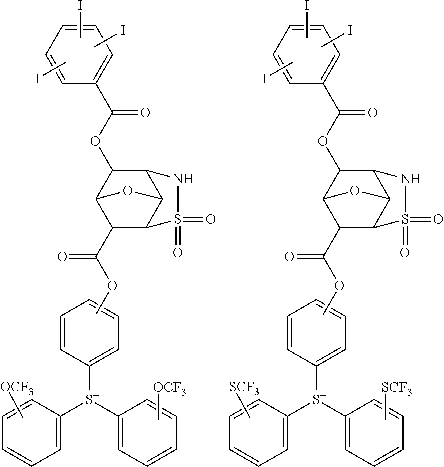

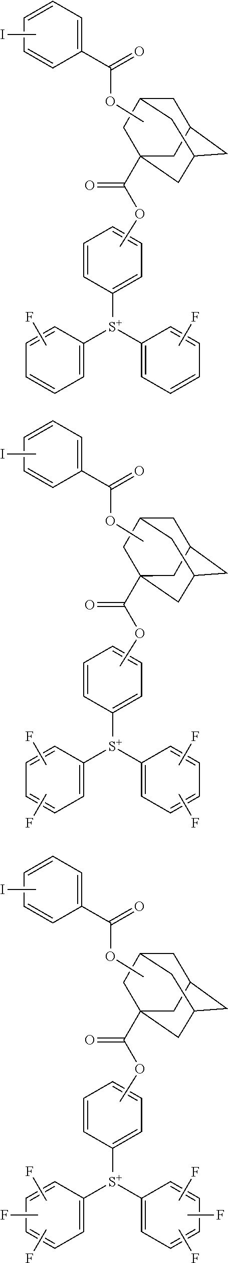

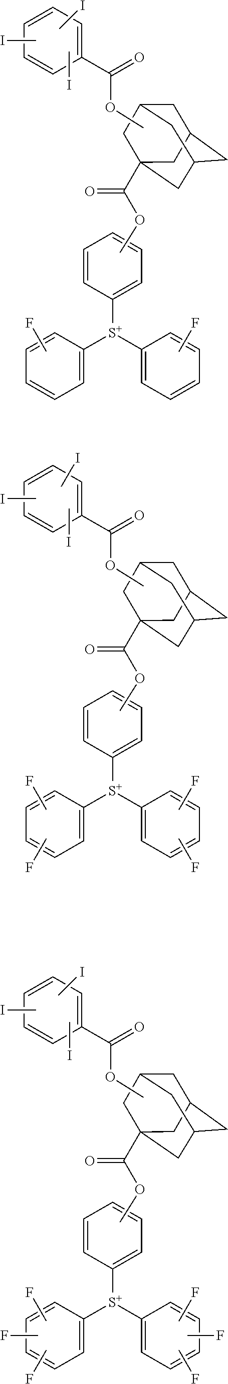

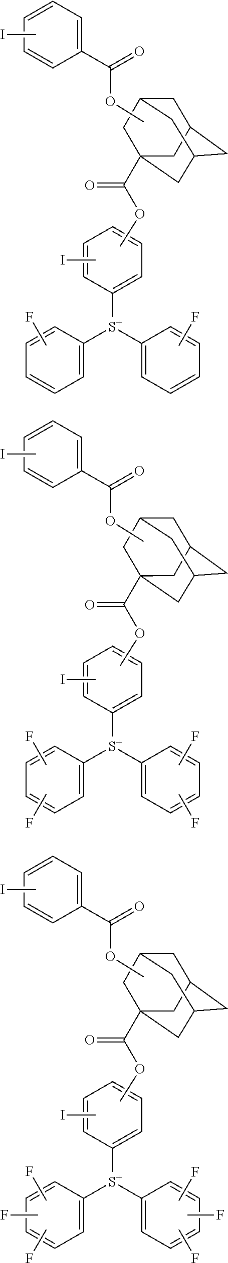

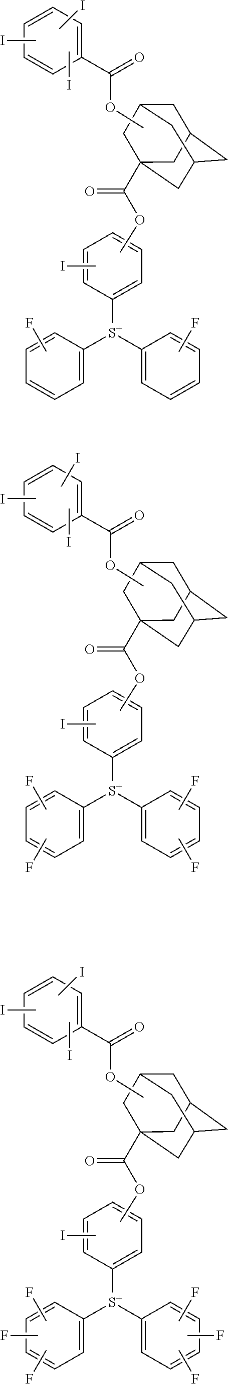

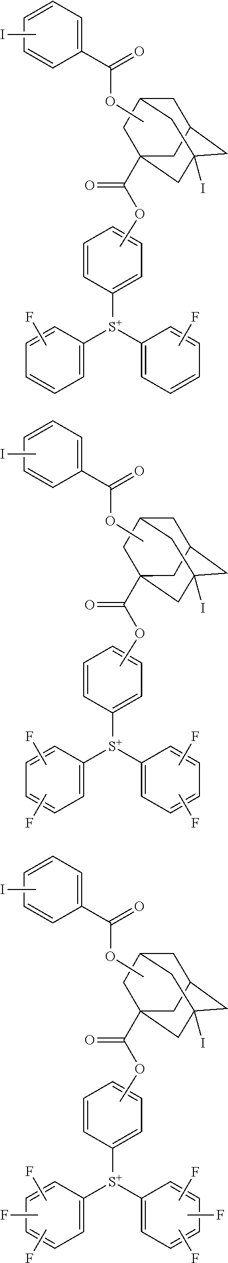

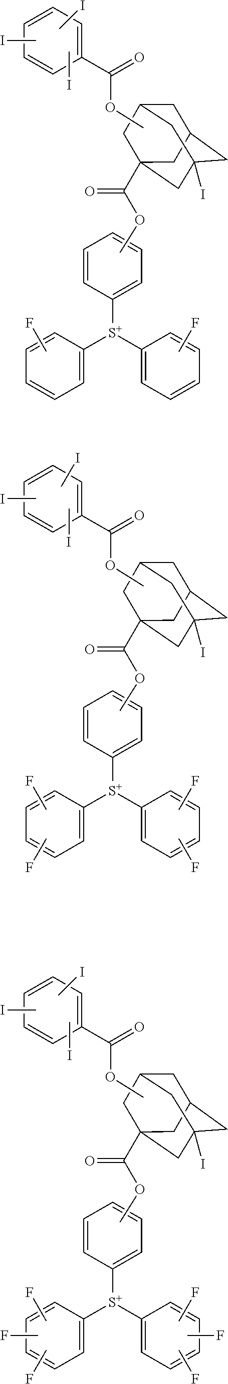

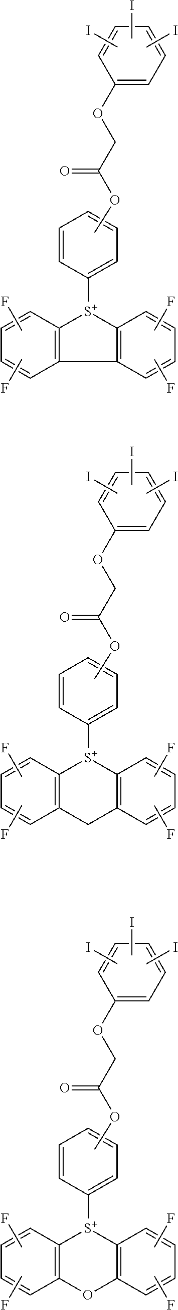

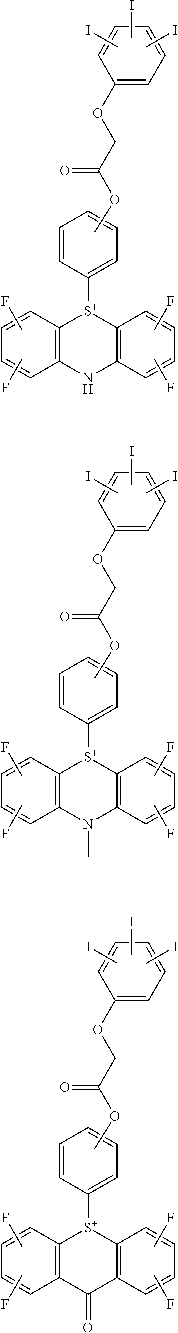

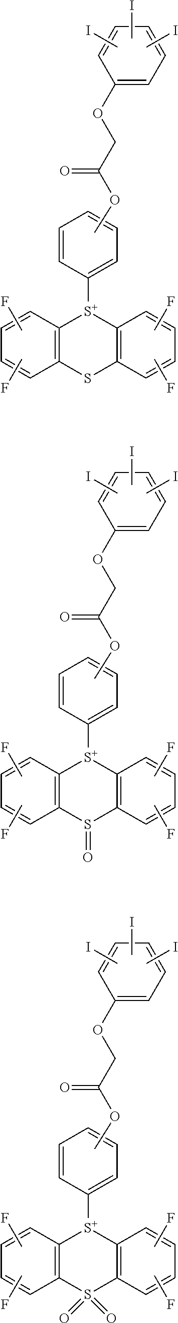

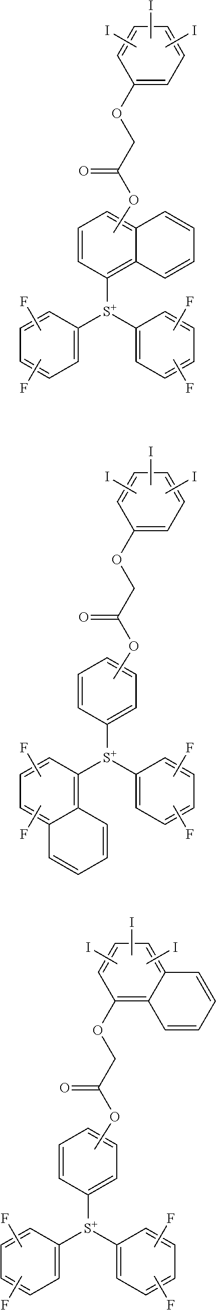

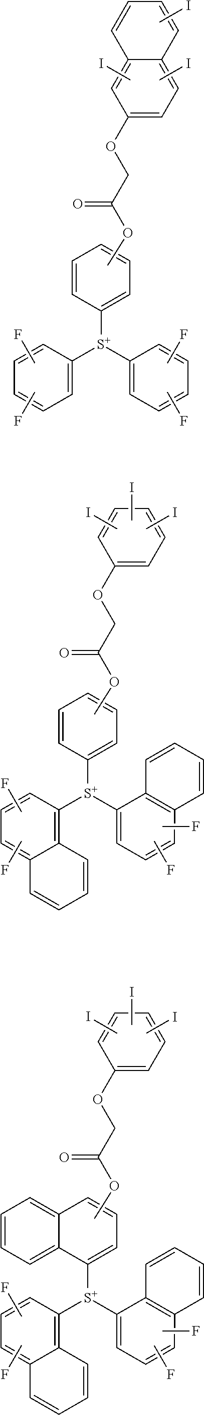

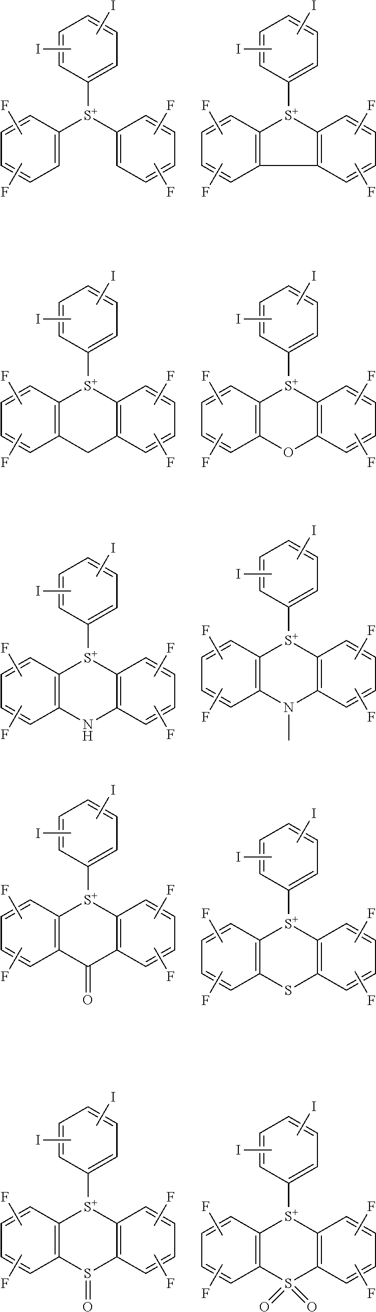

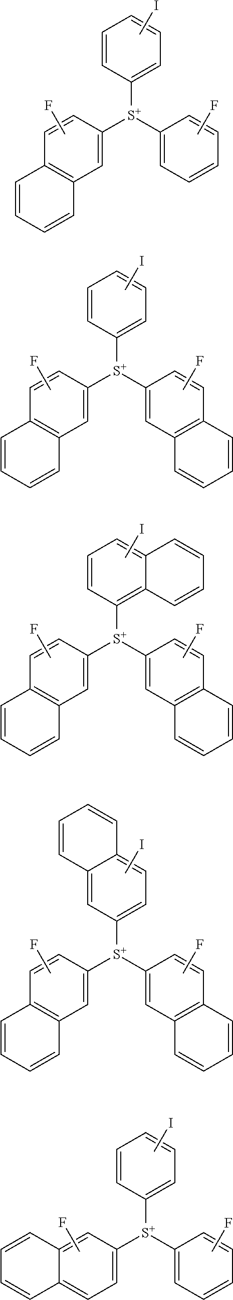

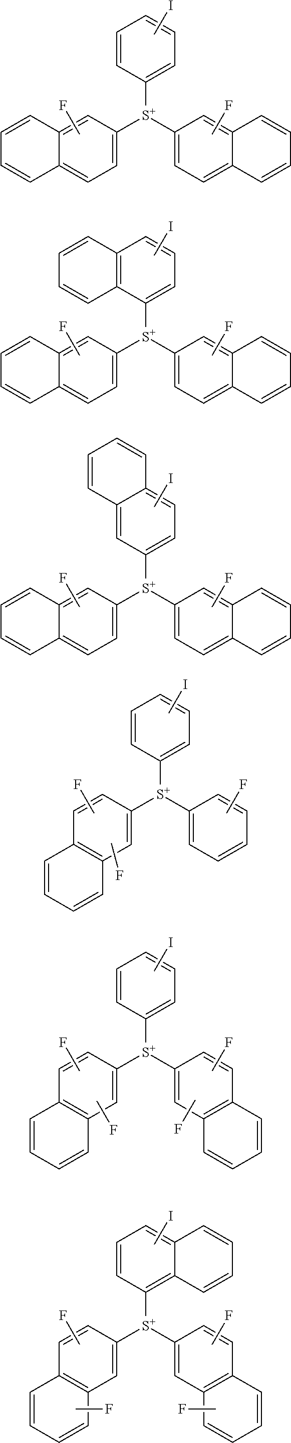

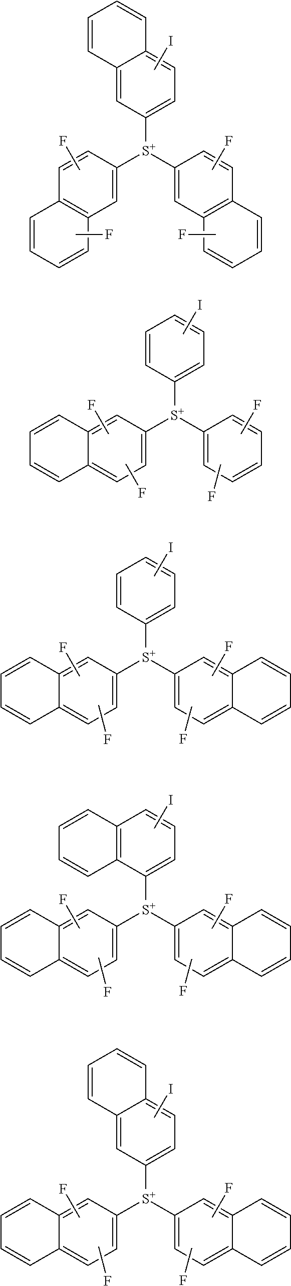

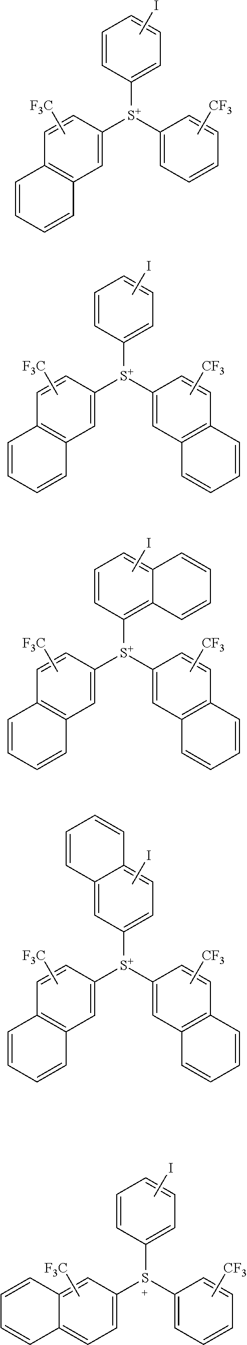

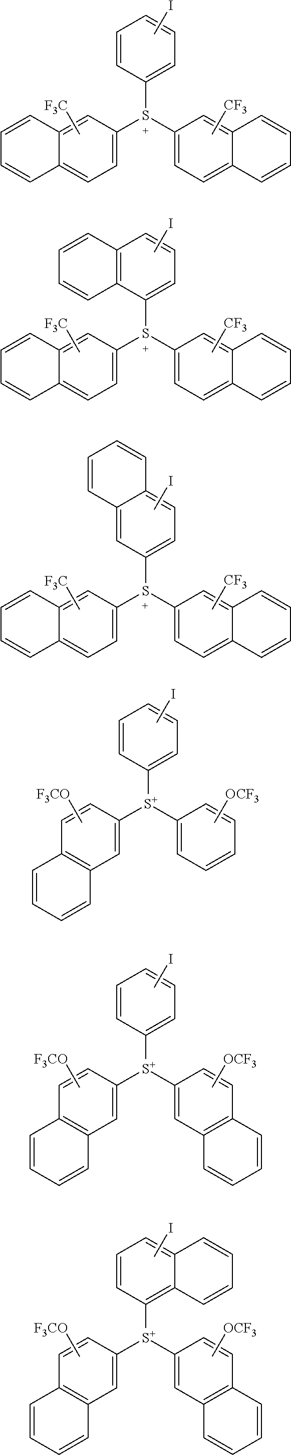

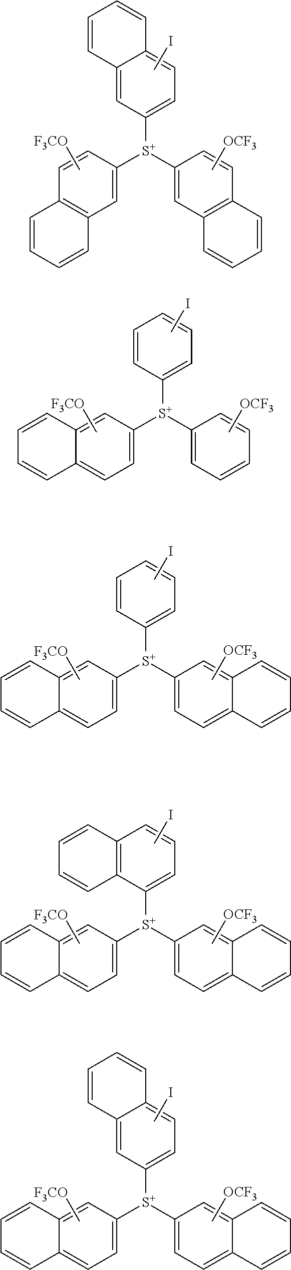

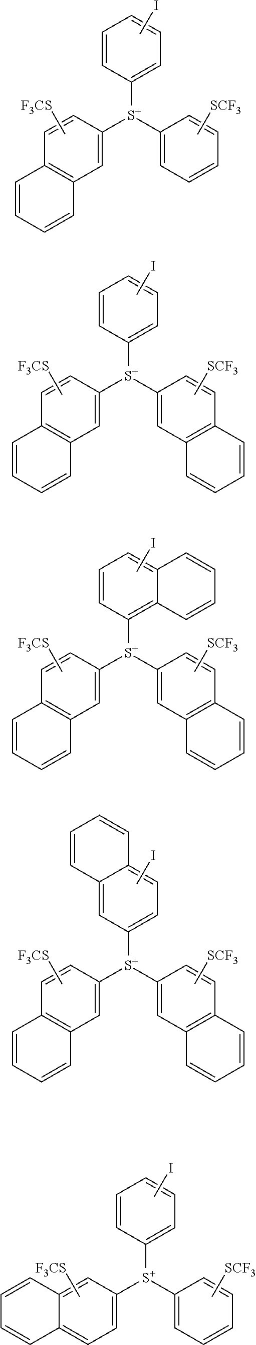

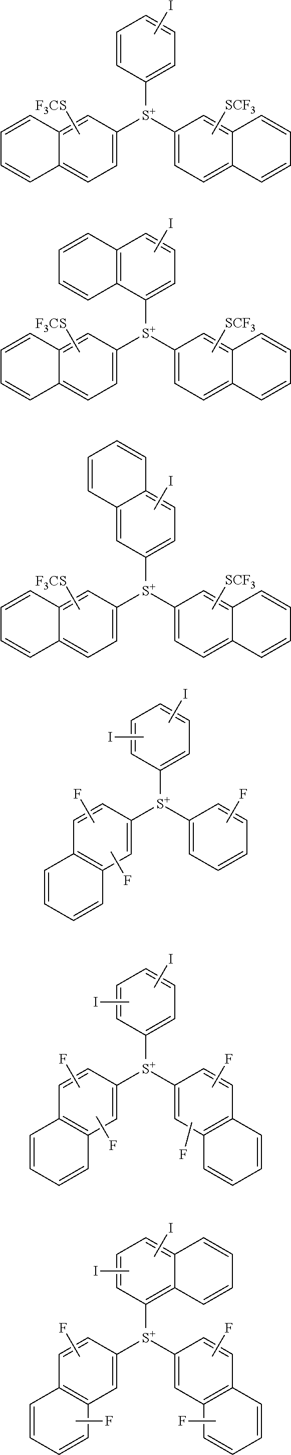

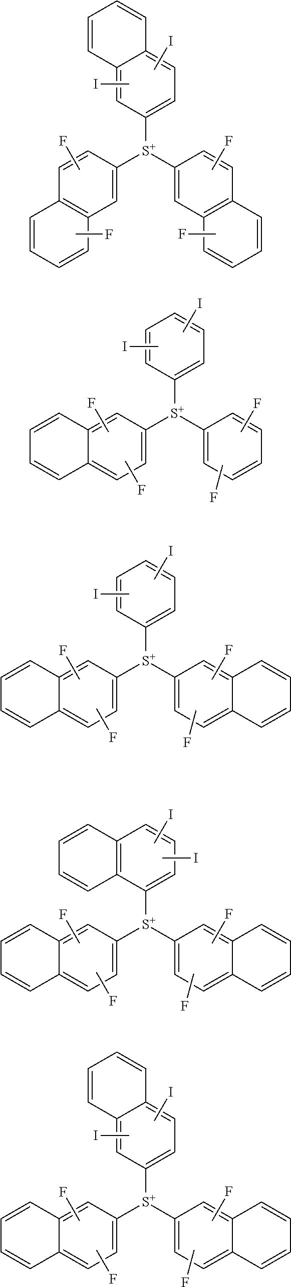

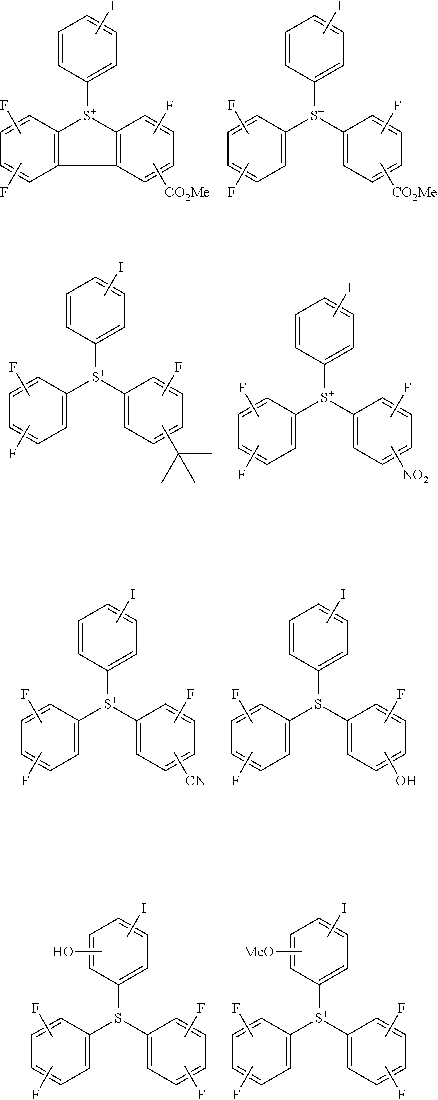

Specific examples of the onium salt include any combination of the foregoing anion and cation.

The onium salt can be synthesized by a known method. As an example, a method of preparing an onium salt having the formula (PAG-1-ex) is described:

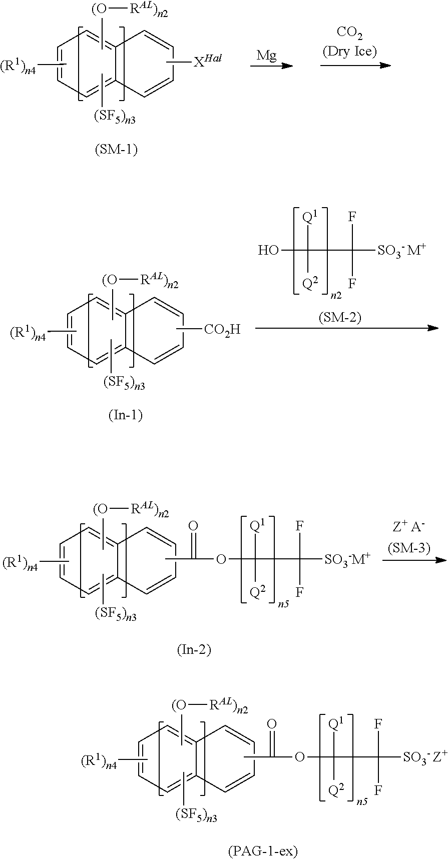

wherein n1 to n5, R^{AL}, R¹, Q¹, Q², and Z⁺ are as defined above, X^{Hal} is a chlorine atom, a bromine atom, or an iodine atom, M⁺ is a counter cation, and X⁻ is a counter anion.

The first step is a step of preparing a Grignard reagent from a commercially available product or reactant SM-1 that can be synthesized by a known synthesis method, and reacting the Grignard reagent with carbon dioxide (dry ice) to obtain Intermediate In-1. The reaction can be performed by a known organic synthesis method. Specifically, metallic magnesium is suspended in an ether solvent such as diethyl ether or tetrahydrofuran (THF), and a diluted solution containing reactant SM-1 and the solvent used is added dropwise to prepare a Grignard reagent. When X^{Hal} of the reactant SM-1 is a bromine atom or an iodine atom, an activator of metallic magnesium is not necessarily required, but when X^{Hal} is a chlorine atom, a Grignard reagent can be smoothly prepared by using a small amount of 1,2-dibromoethane or iodine as an activator. The reaction temperature is preferably from room temperature to the boiling point of the solvent to be used. After preparing the Grignard reagent, the dry ice is suspended in the solvent used for the preparation, and the Grignard reagent is added dropwise. In terms of yield, it is desirable to complete the reaction by tracking the reaction by silica gel thin layer chromatography (TLC), but the reaction time is usually about 5 minutes to 30 minutes. Thereafter, the magnesium salt is dissolved using dilute hydrochloric acid or the like, the target product is extracted from the reaction mixture, and Intermediate In-1 can be obtained by ordinary aqueous work-up. The resulting Intermediate In-1 can be purified by conventional means such as chromatography or recrystallization, if necessary.

The second step is a step of obtaining Intermediate In-2 by the reaction between Intermediate In-1 and reactant SM-2. When an ester bond is directly formed from the carboxy group of Intermediate In-1 and the hydroxy group of reactant SM-2, various condensing agents can be used. Examples of the condensing agent to be used include N,N′-dicyclohexylcarbodiimide, N,N′-diisopropylcarbodiimide, 1-[3-(dimethylamino) propyl]-3-ethylcarbodiimide, and 1-ethyl-3-(3-dimethylaminopropyl) carbodiimide hydrochloride. From the viewpoint of easily removing the urea compound produced as a by-product after the reaction, it is preferable to use 1-ethyl-3-(3-dimethylaminopropyl) carbodiimide hydrochloride. The reaction is performed by dissolving Intermediate In-1 and reactant SM-2 in a halogen solvent such as methylene chloride and adding a condensing agent. When 4-dimethylaminopyridine is added as a catalyst, the reaction rate can be improved. In terms of yield, it is desirable to complete the reaction by tracking the reaction by TLC, but the reaction time is usually about 12 to 24 hours. After the reaction is stopped, if necessary, the urea compound produced as a by-product is removed by filtration or water washing, and then the reaction solution is subjected to ordinary aqueous work-up, obtaining Intermediate In-2. The resulting Intermediate In-2 can be purified by conventional means such as chromatography or recrystallization, if necessary.

The third step is a step of subjecting the resulting Intermediate In-2 to salt exchange with onium salt SM-3 having Z⁺X⁻ to obtain an onium salt (PAG-1-ex). As X⁻, a chloride ion, a bromide ion, an iodide ion, or a methyl sulfate anion is preferred because the exchange reaction easily proceeds quantitatively. The progress of the reaction is desirably confirmed by TLC in terms of yield. The onium salt (PAG-1-ex) can be obtained from the reaction mixture by ordinary aqueous work-up. Purification can be performed by conventional means such as chromatography or recrystallization, if necessary.

In the above reaction scheme, the ion exchange in the third step can be readily performed by any well-known technique, for example, with reference to JP-A 2007-145797.

The above-mentioned preparation method is merely exemplary and the method of preparing the inventive onium salt is not limited thereto.

As a structural feature of the onium salt, the onium salt has an acid labile group and a —SF₅ group bonded to a hydroxy group on an aromatic ring of an anion, and these groups are bonded to adjacent carbon atoms. The acid labile group in the exposed region causes a deprotection reaction by the generated acid, and an aromatic hydroxy group is generated. As a result, the contrast between the exposed region and the unexposed region is improved. The adjacent —SF₅ group improves the resist solvent solubility of the sulfonium salt itself, and also improves the acidity of the aromatic hydroxy group generated in the exposed region due to its strong electron withdrawing property. When the resist film is developed with an alkaline developer after exposure, the affinity between the generated aromatic hydroxy group and the alkaline developer is improved, so that the exposed region is effectively removed by the developer. It is considered that the aromatic hydroxy group adjacent to the —SF₅ group has an effect of reducing swelling caused by the alkaline developer without drawing the alkaline developer to the unexposed region as compared with the carboxy group due to the effect of water repellency of the fluorine atom. As a result, collapse of the resist pattern generated in the unexposed region is suppressed. Due to these synergistic effects, when the onium salt is used, the dissolution contrast is high, LWR of the line pattern or CDU of the hole pattern is improved, and a pattern resistant to pattern collapse can be formed, so that the onium salt is suitable as a positive resist composition.

### Photoacid Generator

The onium salt can be suitably used as a photoacid generator.

### Chemically Amplified Resist Composition

The chemically amplified resist composition comprises (A) the onium salt having formula (1) as a photoacid generator.

In the chemically amplified resist composition, the amount of the photoacid generator in the form of the onium salt having formula (1) as component (A) is preferably 0.1 to 40 parts by weight, more preferably 0.5 to 30 parts by weight per 80 parts by weight of a base polymer to be described below. As long as the amount of component (A) is in the range, high sensitivity and resolution are achievable and the risk of foreign matter being formed after development or during stripping of resist film is avoided, which is preferable. The photoacid generator (A) may be used alone or in admixture.

### (B) Base Polymer

The chemically amplified resist composition may comprise a base polymer as component (B). Examples of the base polymer (B) include base polymers comprising a repeat unit having the formula (a1)(hereinafter, the repeat unit is also referred to as a repeat unit a1) or a repeat unit having the formula (a2)(hereinafter, the repeat unit is also referred to as a repeat unit a2).

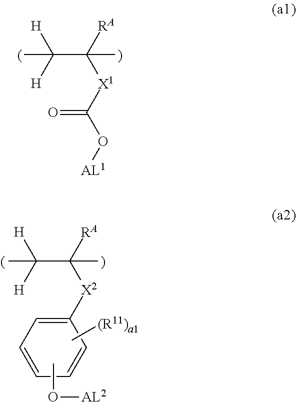

In formulae (a1) and (a2), R^{A} is each independently a hydrogen atom, a fluorine atom, a methyl group, or a trifluoromethyl group.

In formula (a1), X¹ is a single bond, a phenylene group, a naphthylene group, *—C(═O)—O—X¹¹—, or *—C(═O)—NH—X¹¹—, and the phenylene group or the naphthylene group may be substituted with a hydroxy group, a nitro group, a cyano group, an optionally fluorinated C₁-C₁₀ saturated hydrocarbyl group, an optionally fluorinated C₁-C₁₀ saturated hydrocarbyloxy group, or a halogen atom. X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring. * designates a point of attachment to the carbon atom in the backbone.

In formula (a2), X² is a single bond, *—C(═O)—O—, or *—C(═O)—NH—.

* designates a point of attachment to the carbon atom in the backbone. R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. a1 is 0, 1, 2, 3, or 4, preferably 0 or 1.

In formulae (a1) and (a2), A^{L1} and A^{L2} are each independently an acid labile group. Specific examples of the acid labile group are described in JP-A 2013-80033 and JP-A 2013-83821.

Typically, specific examples of the acid labile group include acid labile groups having the formulae (AL-3) to (AL-5).

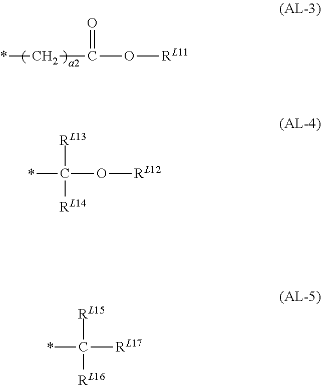

Herein the broken line designates a point of attachment.

In formulae (AL-3) and (AL-4), R^{L11} and R^{L12} are each independently a C₁-C₄₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group preferably contains 1 to 20 carbon atoms.

In formula (AL-3), a2 is an integer of 0 to 10, preferably 1, 2, 3, 4, or 5.

In formula (AL-4), R^{L13} and R^{L14} are each independently a hydrogen atom or a C₁-C₂₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group preferably contains 1 to 20 carbon atoms. Any two of R^{L12}, R^{L13}, and R^{L14} may bond together to form a C₃-C₂₀ ring with the carbon atom or the carbon atom and the oxygen atom to which they are attached. The ring is preferably a C₄-C₁₆ ring, particularly preferably an alicyclic ring.

In formula (AL-5), R^{L15}, R^{L16}, and R^{L17} are each independently a C₁-C₂₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group preferably contains 1 to 20 carbon atoms. Any two of R^{L15}, R^{L16}, and R^{L17} may bond together to form a C₃-C₂₀ ring with the carbon atom to which they are attached. The ring is preferably a C₄-C₁₆ ring, particularly preferably an alicyclic ring.

Specific examples of the repeat unit a1 are shown below, but not limited thereto. Herein R^{A} and A^{L1} are as defined above.

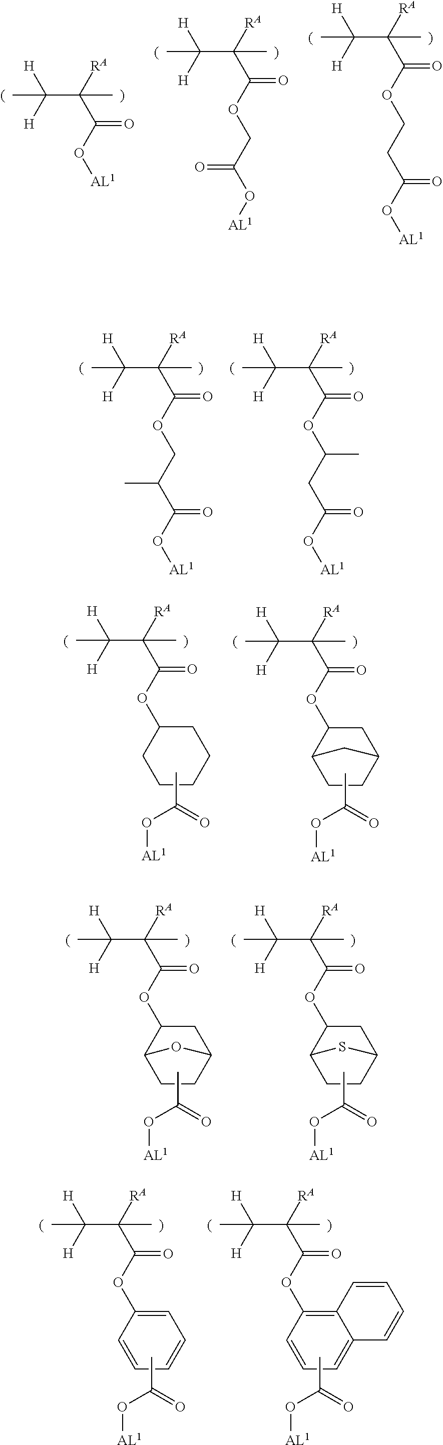

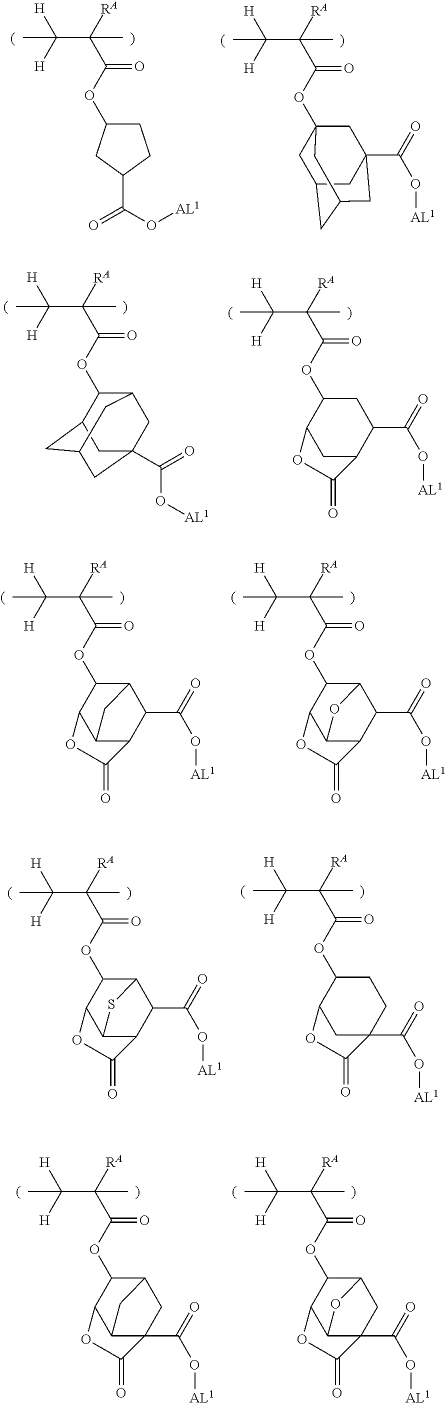

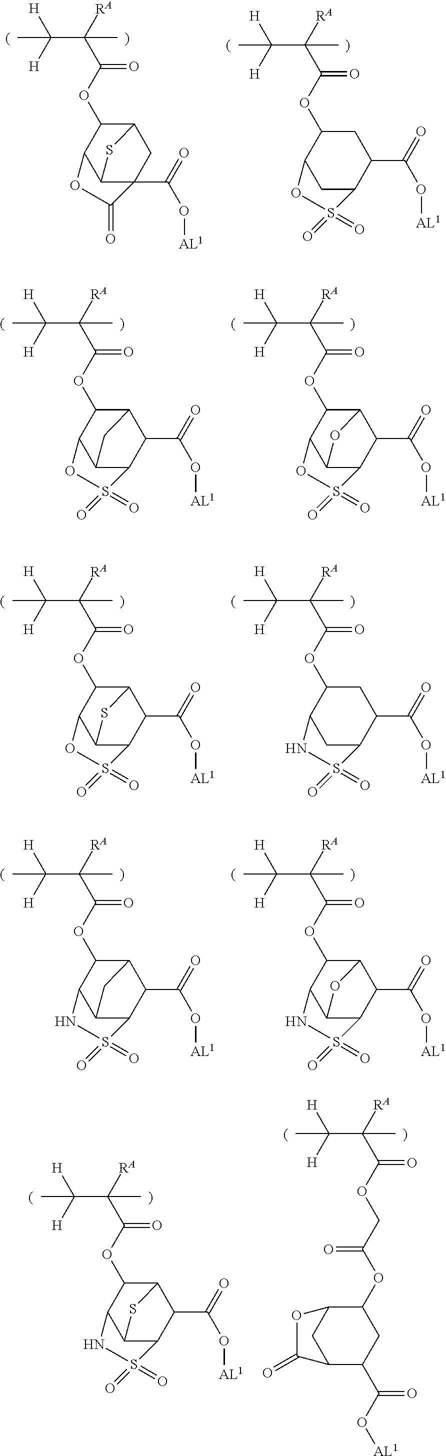

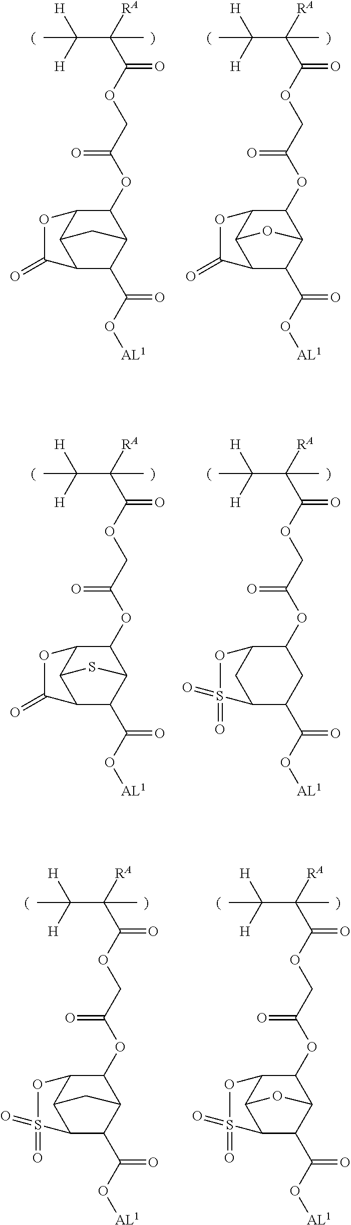

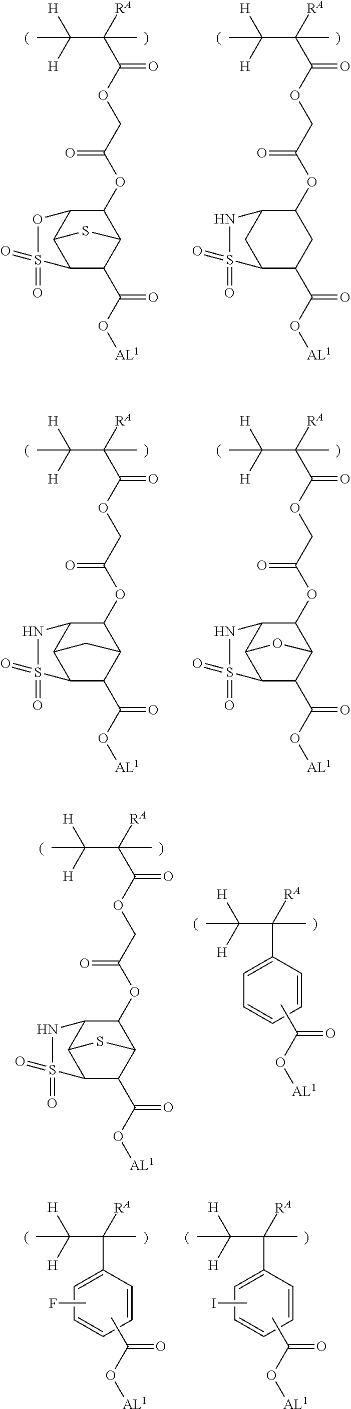

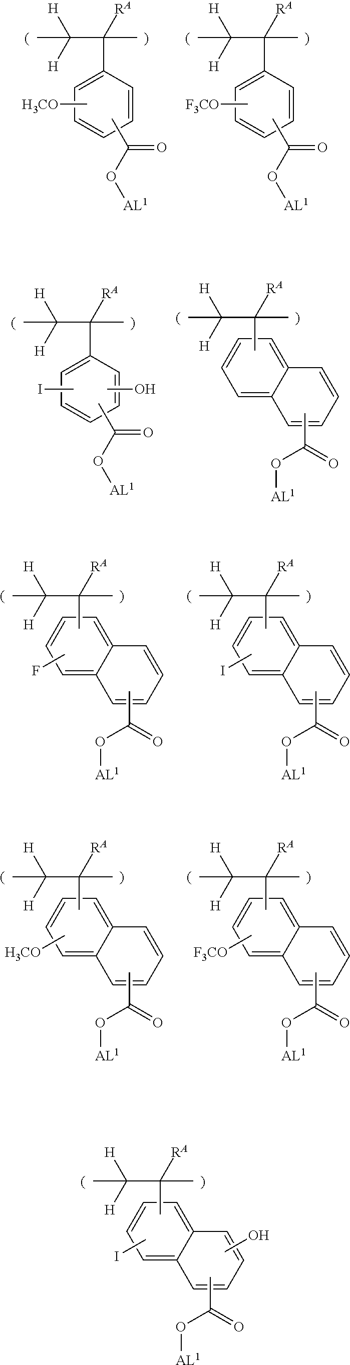

Specific examples of the repeat unit a2 are shown below, but not limited thereto. Herein R^{A} and A^{L2} are as defined above.

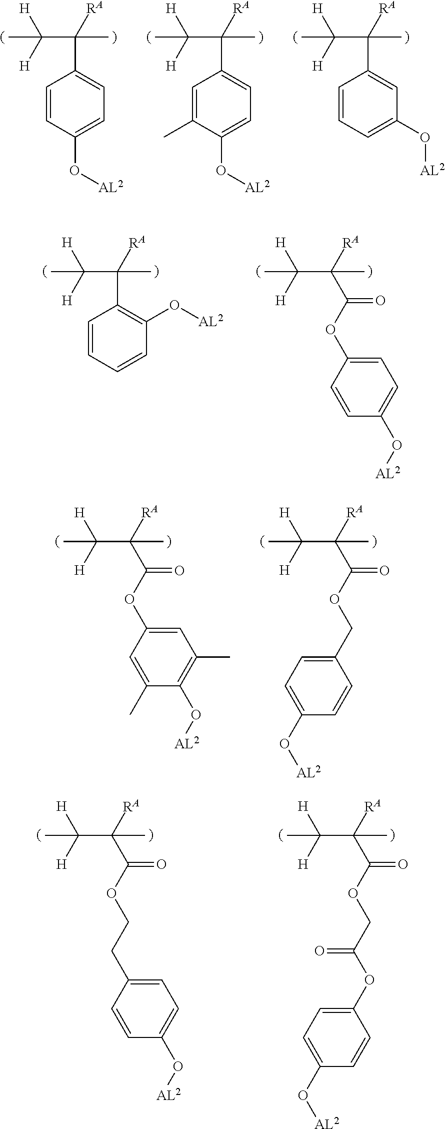

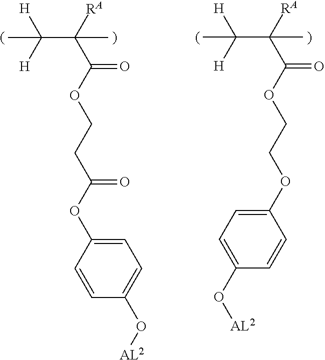

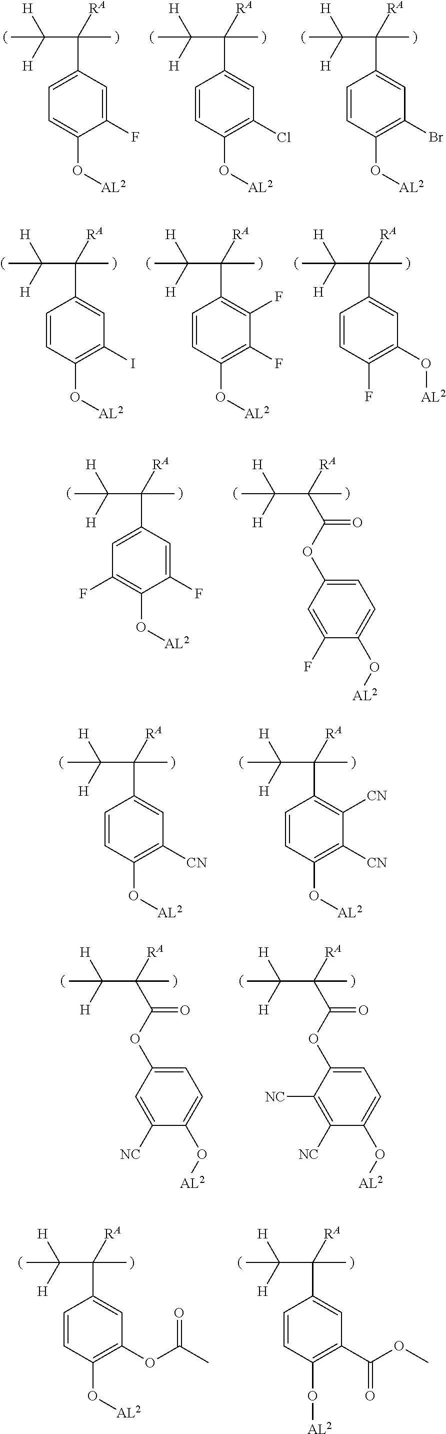

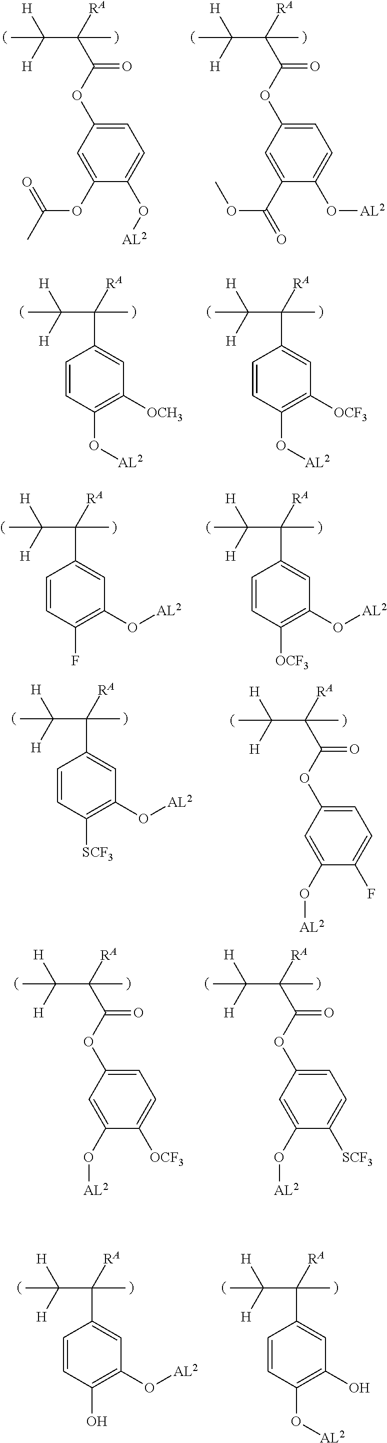

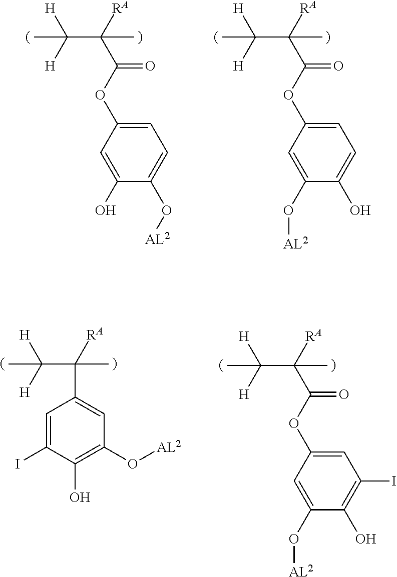

The base polymer may comprise a repeat unit having the formula (a3)(hereinafter, the repeat unit is also referred to as a repeat unit a3).

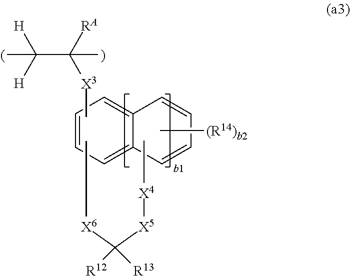

In formula (a3), b1 is 0 or 1. The relevant structure is a benzene ring when b1 is 0, and a naphthalene ring when b1 is 1. From the viewpoint of solvent solubility, the benzene ring corresponding to b1=0 is preferred. b2 is 0, 1, 2, or 3 when b1 is 0, and is 0, 1, 2, 3, 4, or 5 when b1 is 1. From the viewpoint of reactant availability, b2 is preferably 0, 1, 2, or 3, more preferably 0, 1, or 2.

In formula (a3), R^{A} is a hydrogen atom, a fluorine atom, a methyl group, or a trifluoromethyl group. Inter alia, a hydrogen atom and a methyl group are preferred, and a hydrogen atom is more preferred.

In formula (a3), X³ is a single bond, *—C(═O)—O—, or *—C(═O)—NH—. * designates a point of attachment to the carbon atom in the backbone. Inter alia, a single bond and *—C(═O)—O—are preferred, and a single bond is more preferred.

In formula (a3), X⁴ is a single bond, a C₁-C₄ aliphatic hydrocarbylene group, a carbonyl group, a sulfonyl group, or a group obtained by combining the foregoing. Inter alia, a single bond, a carbonyl group, or a sulfonyl group is preferred from the viewpoint of reactant availability, and a single bond or a carbonyl group is more preferred from the viewpoint of a polar group generated after the reaction.

In formula (a3), X⁵ and X⁶ are each independently an oxygen atom or a sulfur atom. Provided that X⁴ and X⁶ bond to adjacent carbon atoms of the aromatic ring. X⁵ and X⁶ may be identical or different, but X⁵ and X⁶ are both preferably an oxygen atom from the viewpoint of reactivity.

In formula (a3), R¹² and R¹³ are each independently a hydrogen atom or a C₁-C₂₀ hydrocarbyl group which may contain a heteroatom. The hydrocarbyl group may be saturated or unsaturated and straight, branched, or cyclic. Specific examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. In the hydrocarbyl group, some or all of the hydrogen atoms may be substituted by a moiety containing a heteroatom such as an oxygen atom, a sulfur atom, a nitrogen atom, or a halogen atom, and some constituent —CH₂— may be replaced by a moiety containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

R¹² and R¹³ may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. In the ring, some or all of the hydrogen atoms may be substituted by a moiety containing a heteroatom such as an oxygen atom, a sulfur atom, a nitrogen atom, or a halogen atom, and some constituent —CH₂— may be replaced by a moiety containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

In formula (a3), R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}). R^{14A} to R^{14B} are each independently a hydrogen atom or a C₁-C₆ hydrocarbyl group. The halogen atom is preferably a fluorine atom, a chlorine atom, a bromine atom, or an iodine atom, more preferably a fluorine atom or an iodine atom. The hydrocarbyl group and the hydrocarbyl moiety of the hydrocarbyloxy group, the hydrocarbyloxycarbonyl group, and the hydrocarbylthio group may be saturated or unsaturated and straight, branched, or cyclic. Specific examples thereof are as exemplified above for the hydrocarbyl groups R¹² and R¹³. In the hydrocarbyl group, some or all of the hydrogen atoms may be substituted by a moiety containing a heteroatom such as an oxygen atom, a sulfur atom, a nitrogen atom, or a halogen atom, and some constituent —CH₂—may be replaced by a moiety containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group. When b2 is 2 or more, R¹⁴ may be identical or different.

When b2 is 2 or more, a plurality of R¹⁴ may bond together to form a ring with the carbon atom of the aromatic ring to which they are attached. Specific examples of the ring formed at this time include a cyclopropane ring, a cyclobutane ring, a cyclopentane ring, a cyclohexane ring, a norbornane ring, and an adamantane ring. In the ring, some or all of the hydrogen atoms may be substituted by a moiety containing a heteroatom such as an oxygen atom, a sulfur atom, a nitrogen atom, or a halogen atom, and some constituent —CH₂—may be replaced by a moiety containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

Specific examples of the repeat unit a3 are shown below, but not limited thereto. Herein R^{A} is as defined above, and Me is a methyl group. The bond positions of substituents on the aromatic ring are interchangeable.

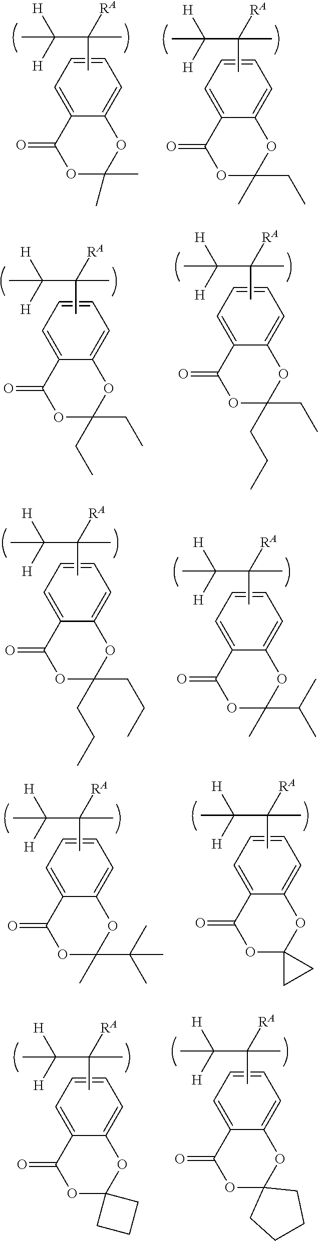

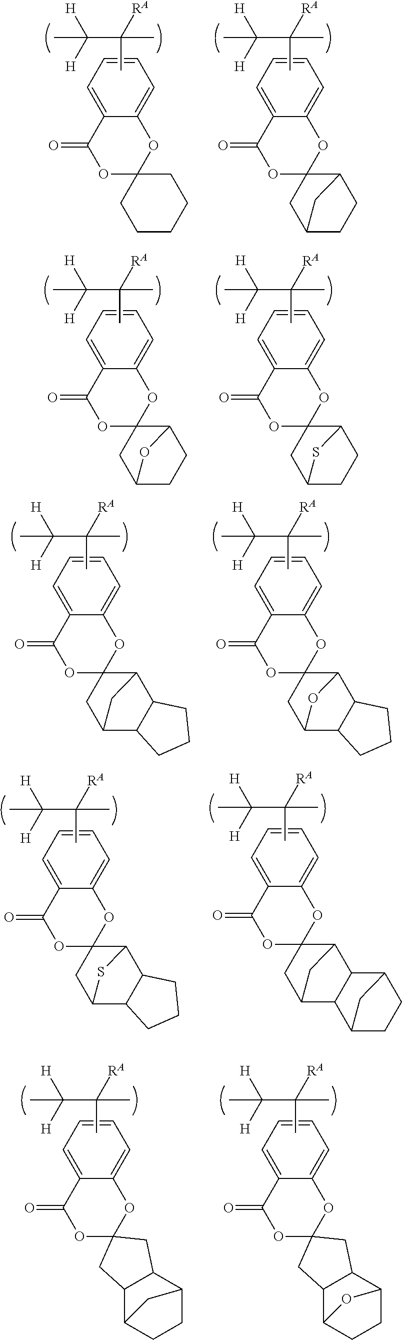

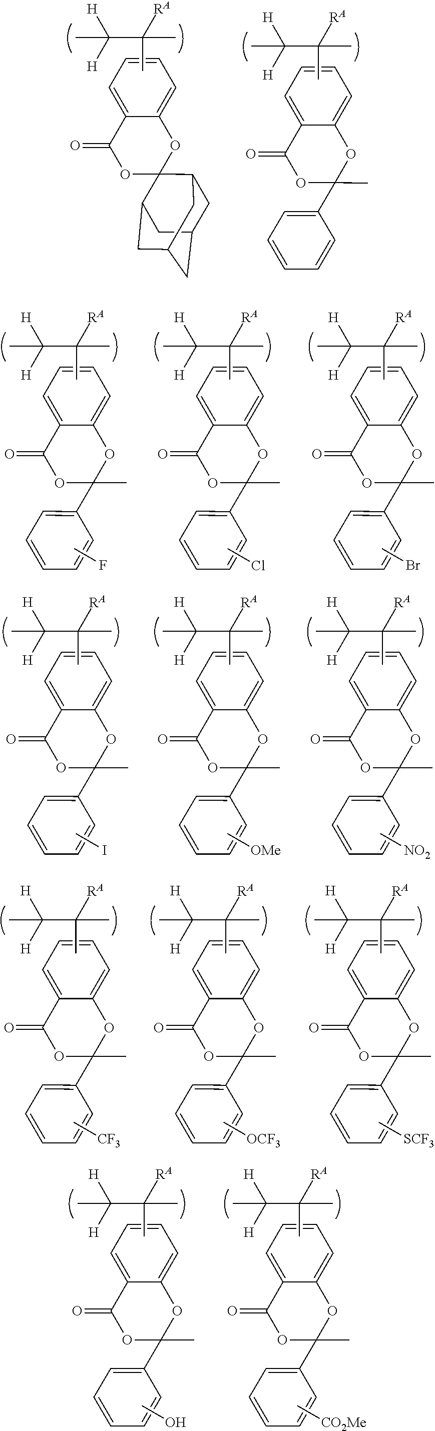

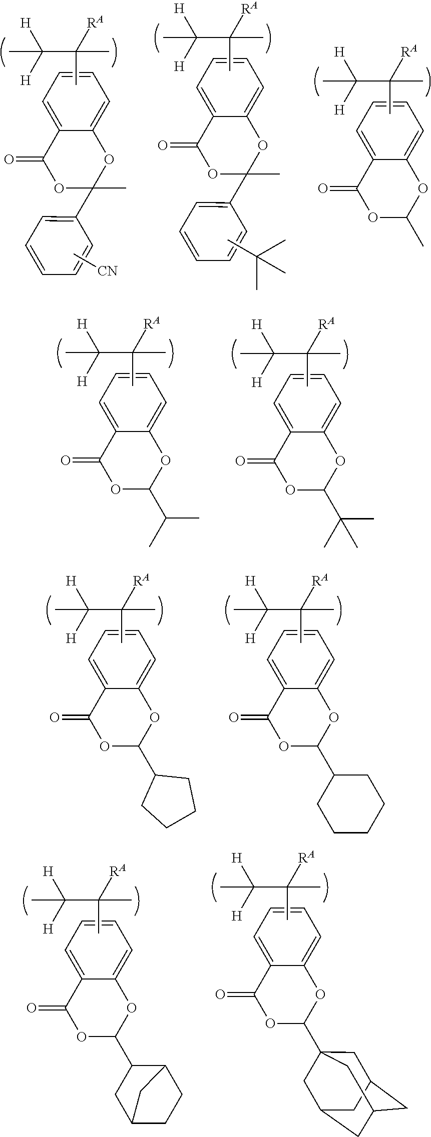

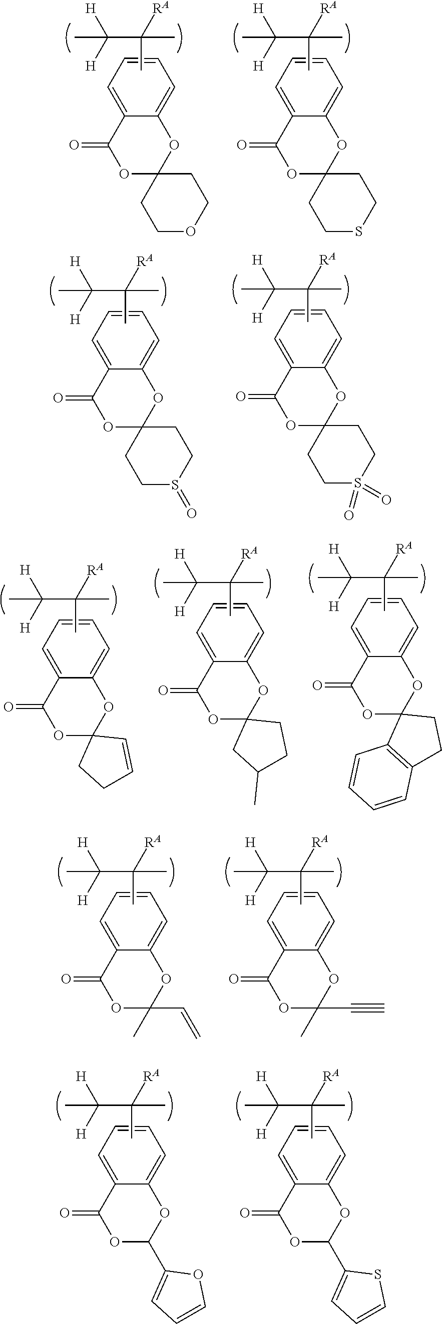

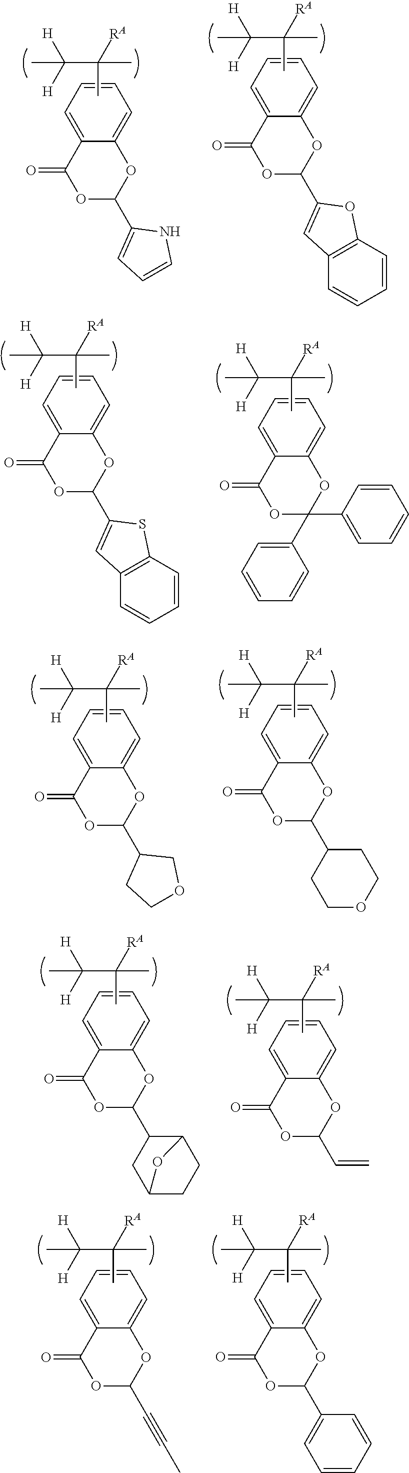

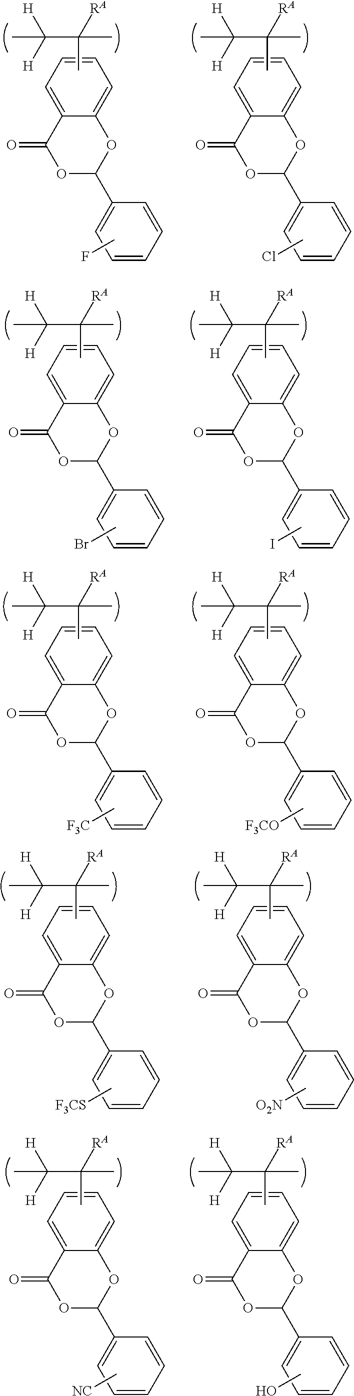

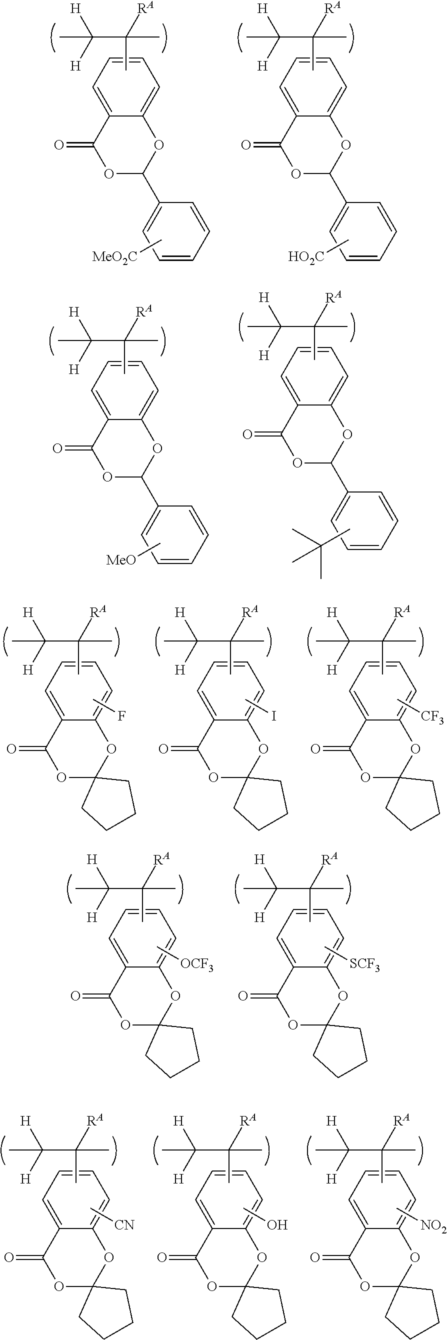

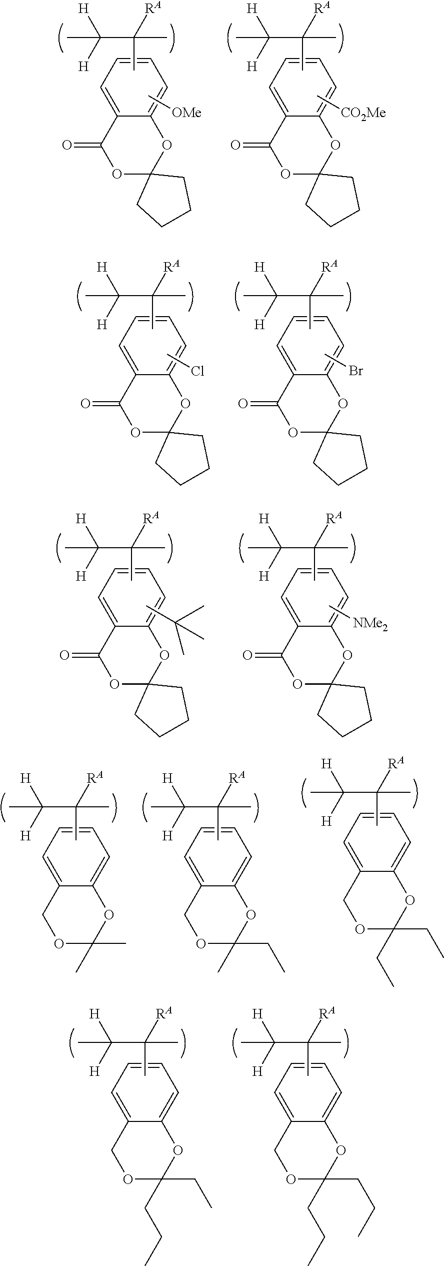

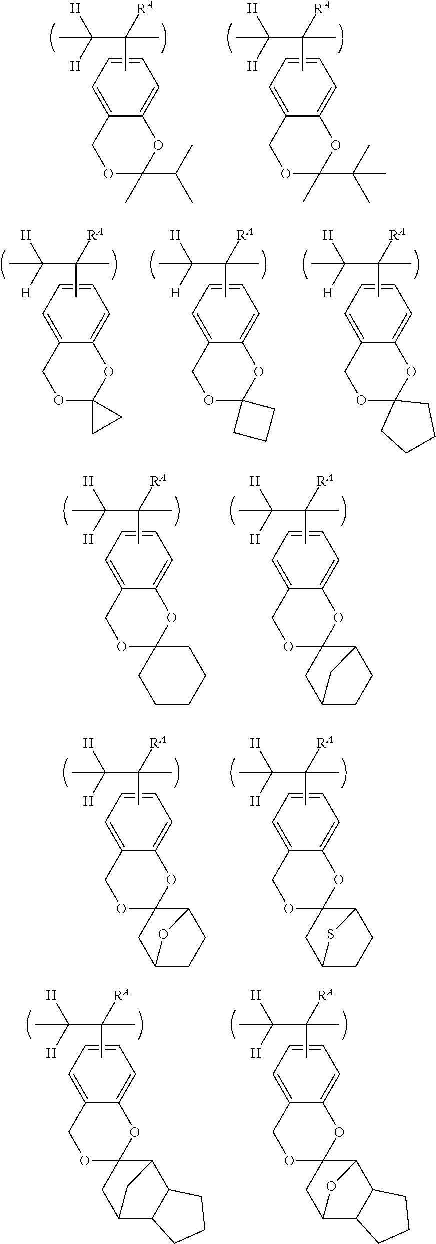

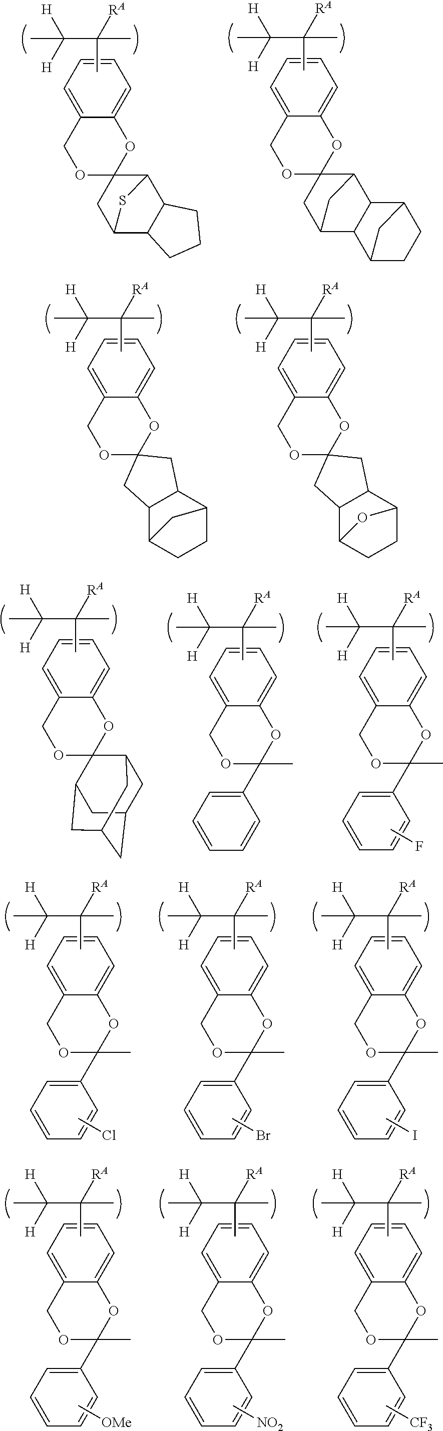

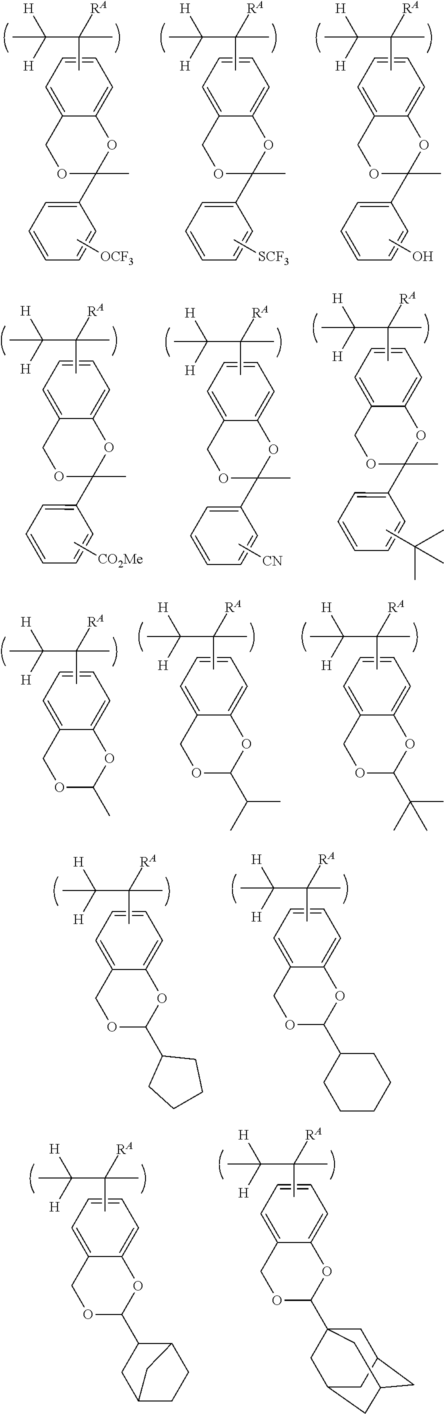

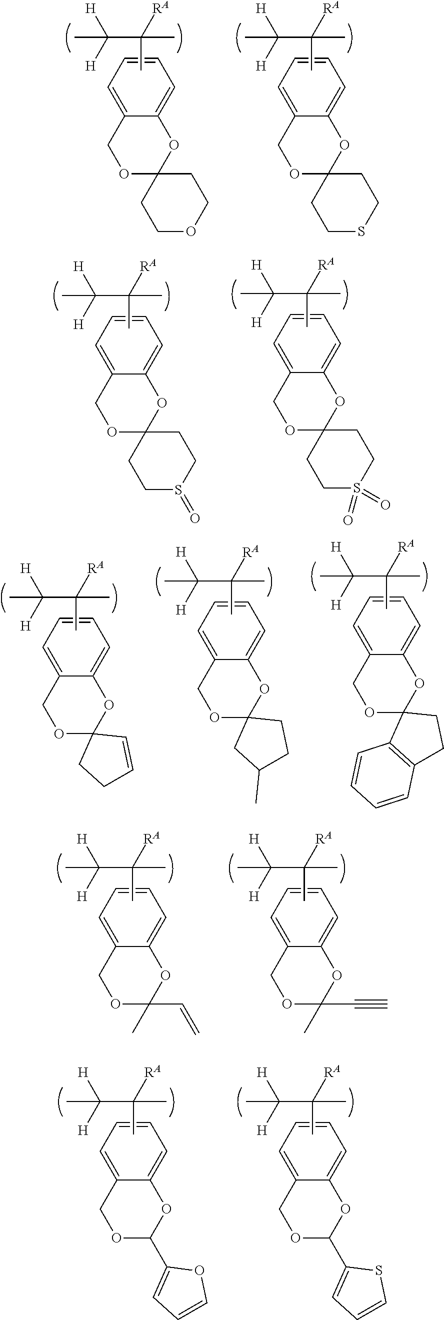

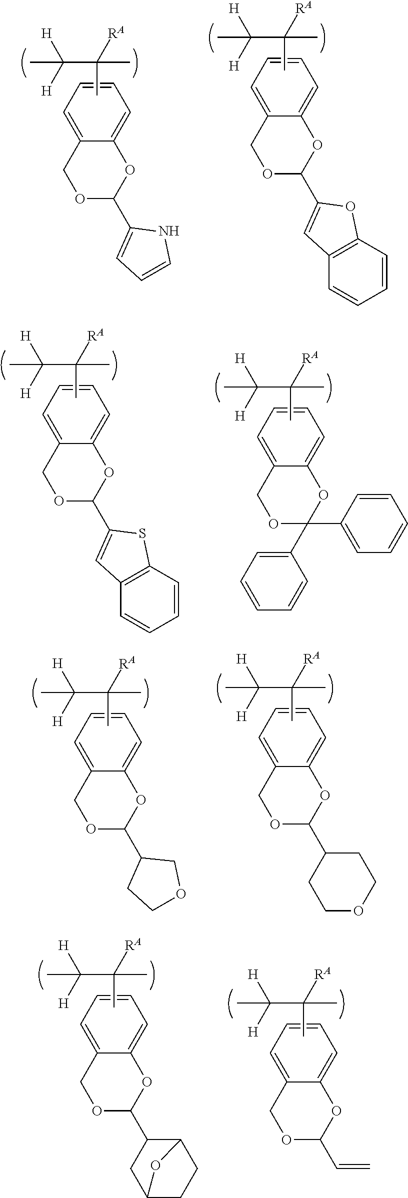

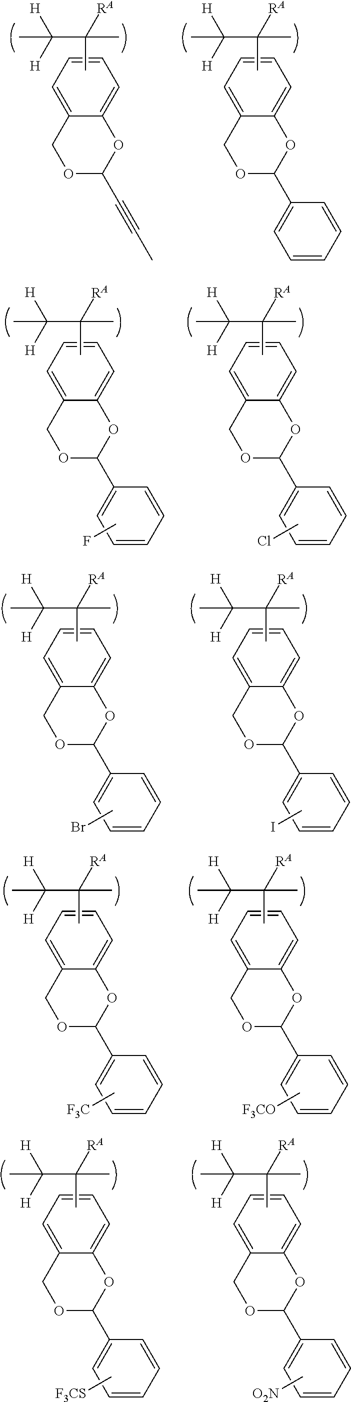

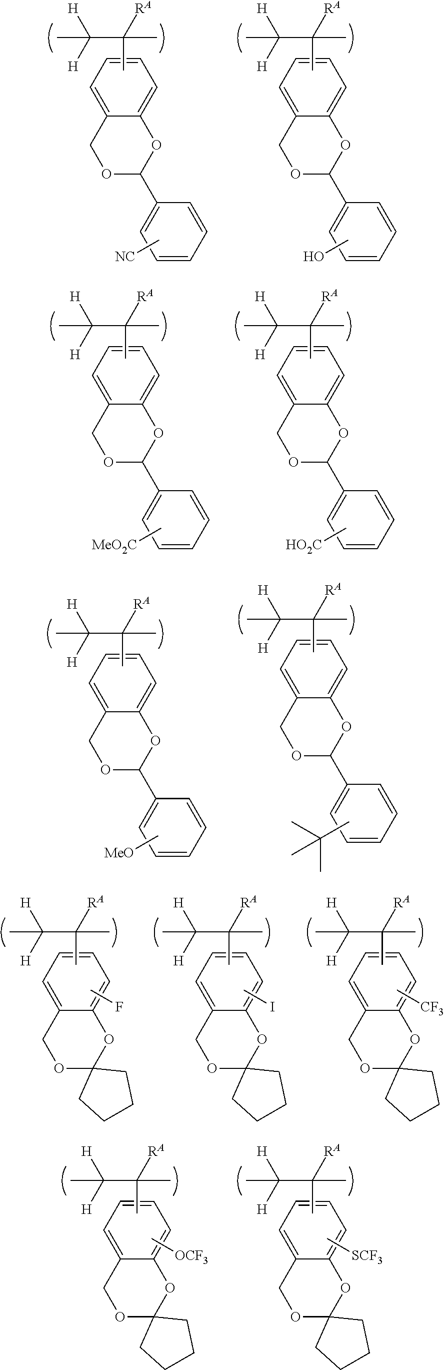

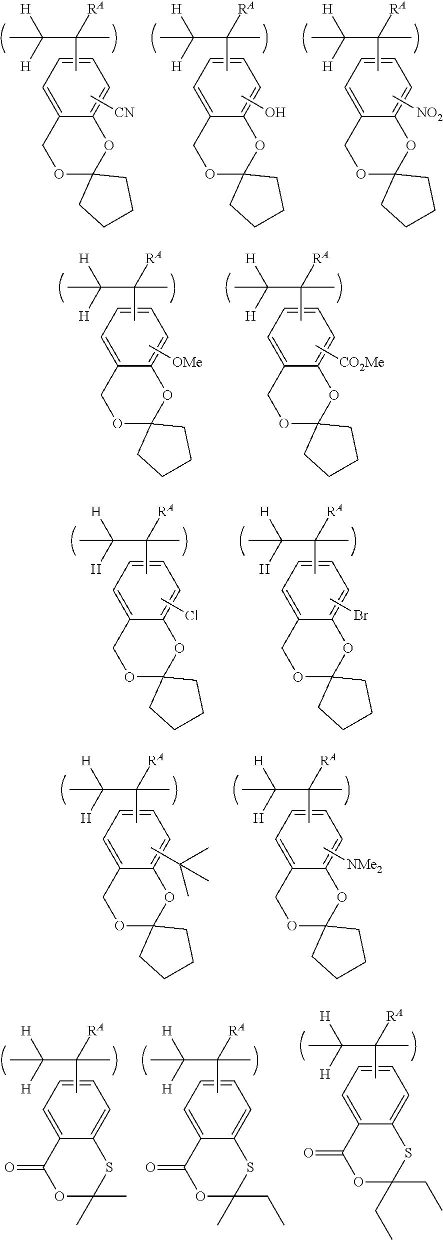

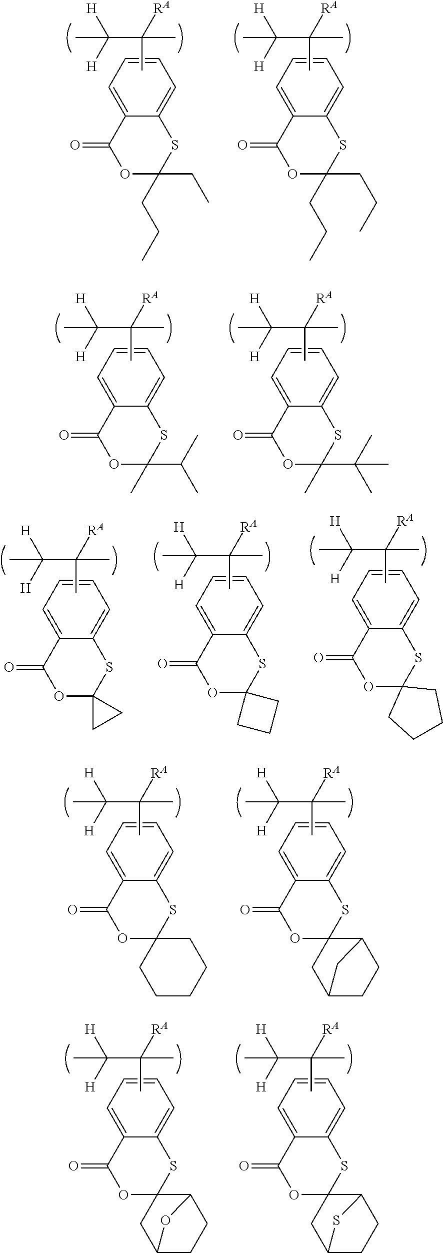

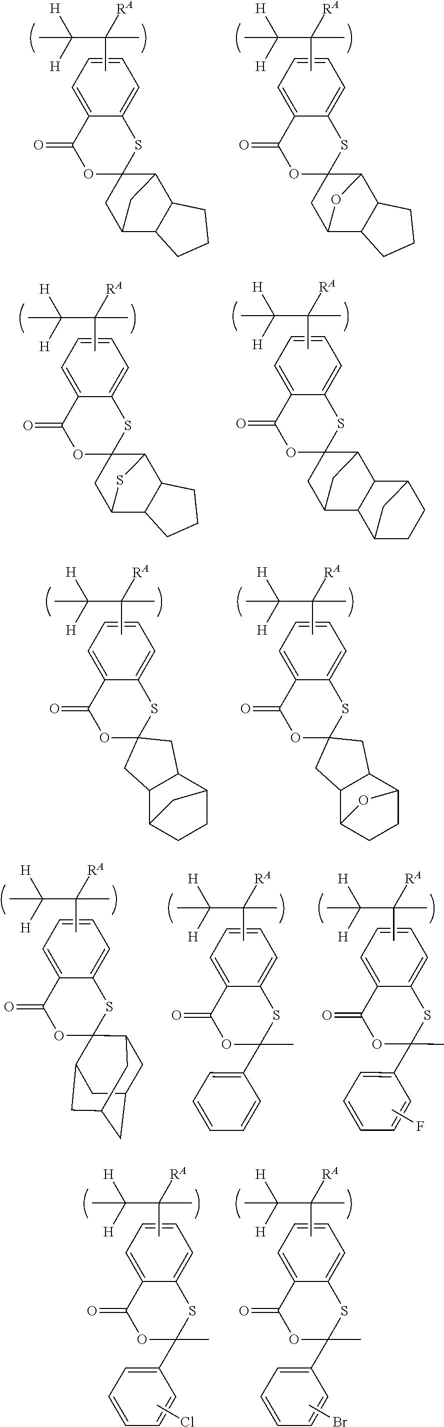

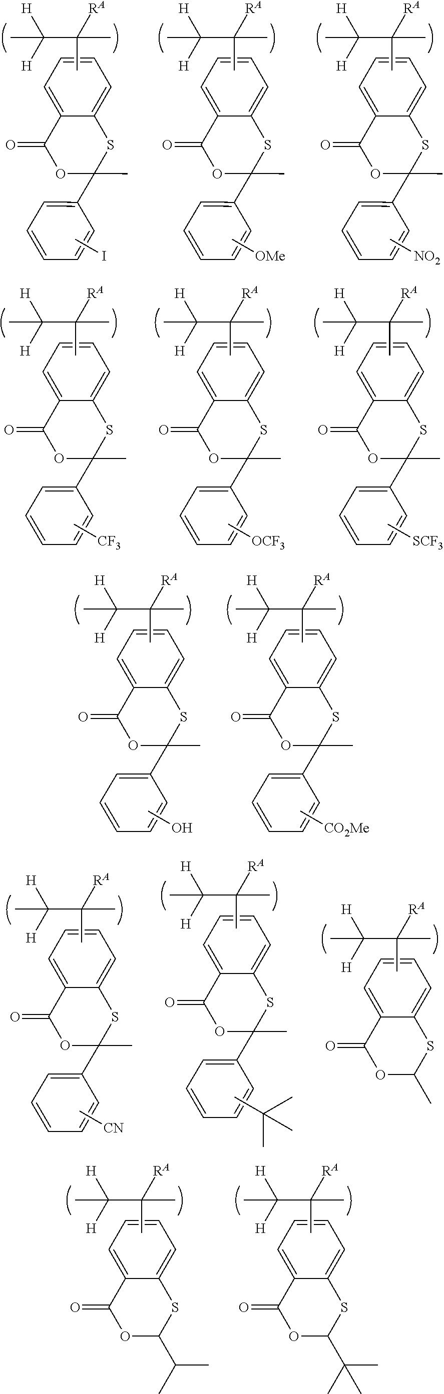

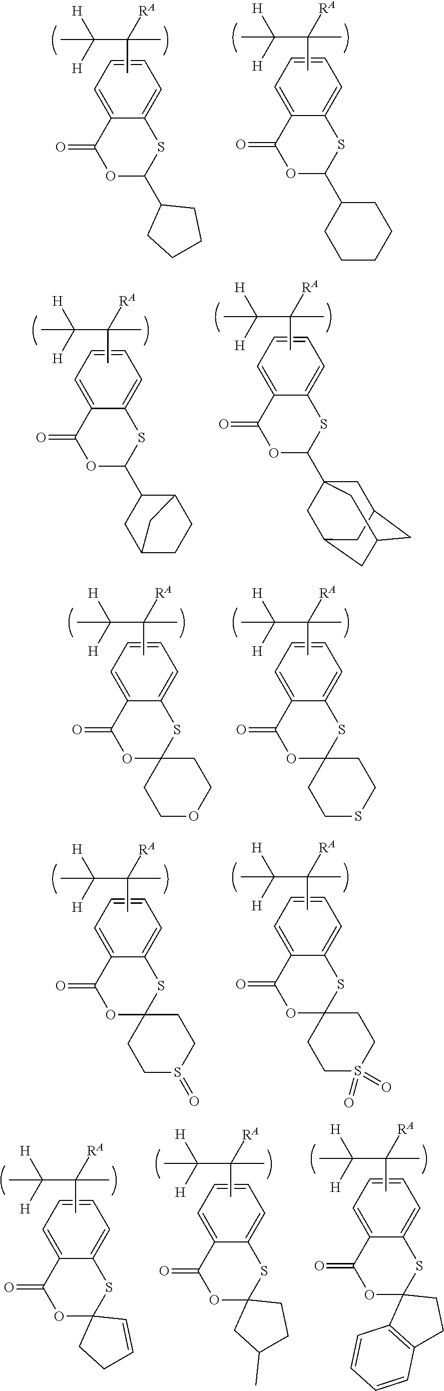

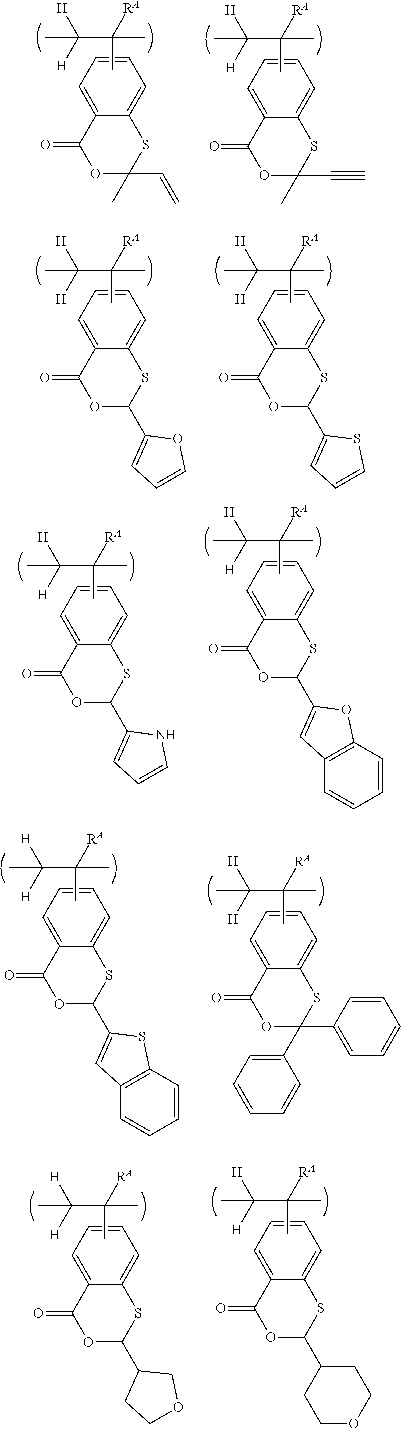

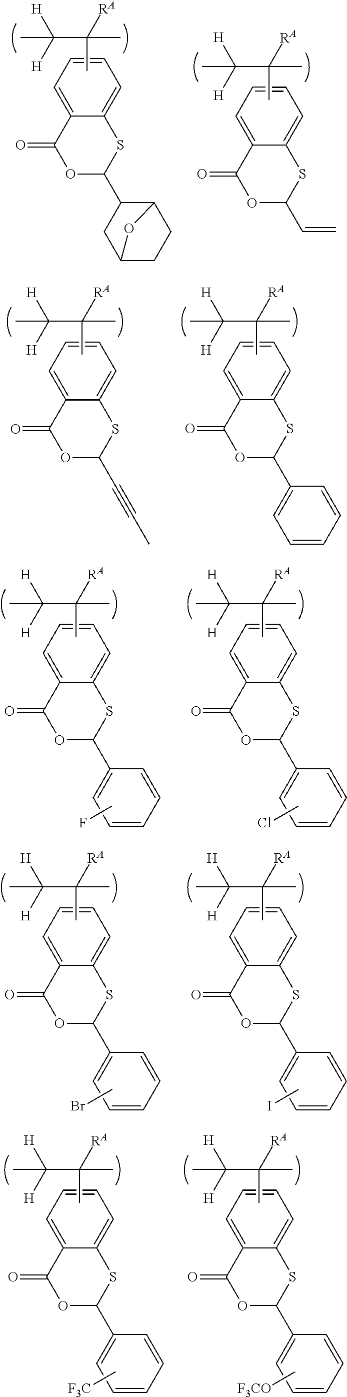

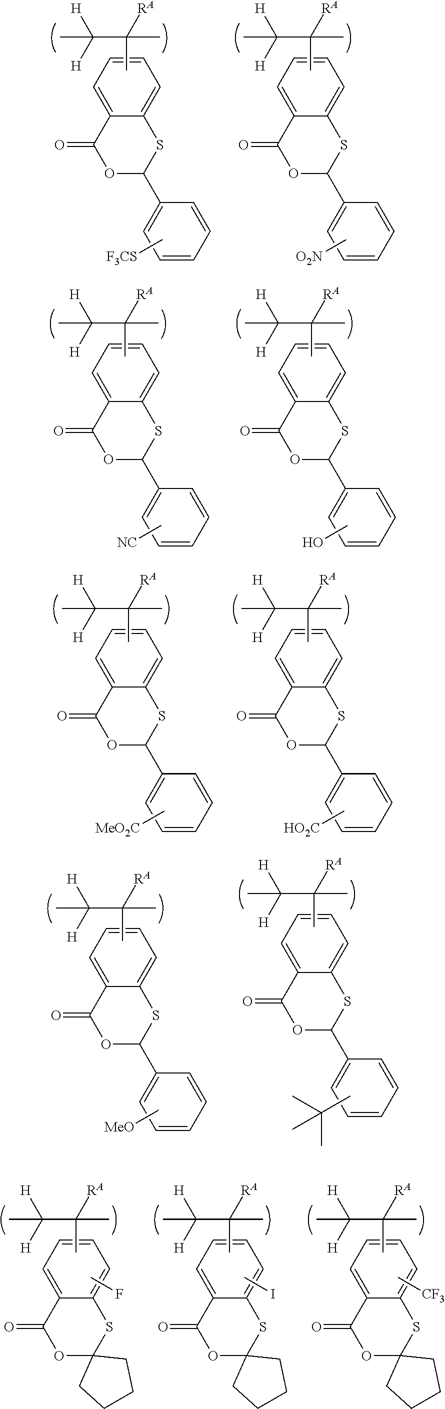

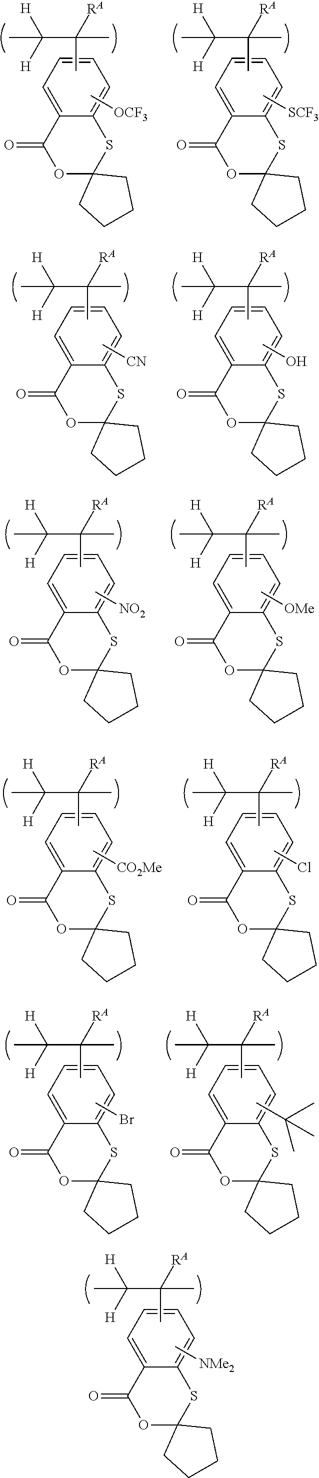

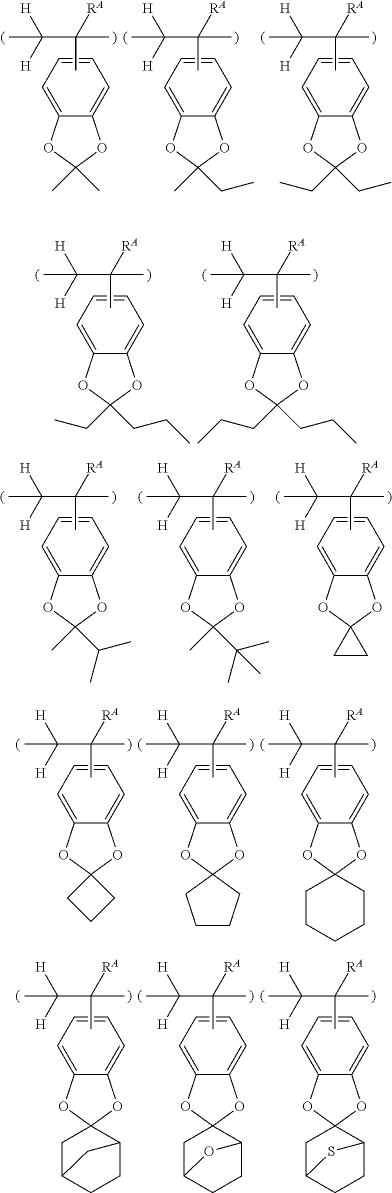

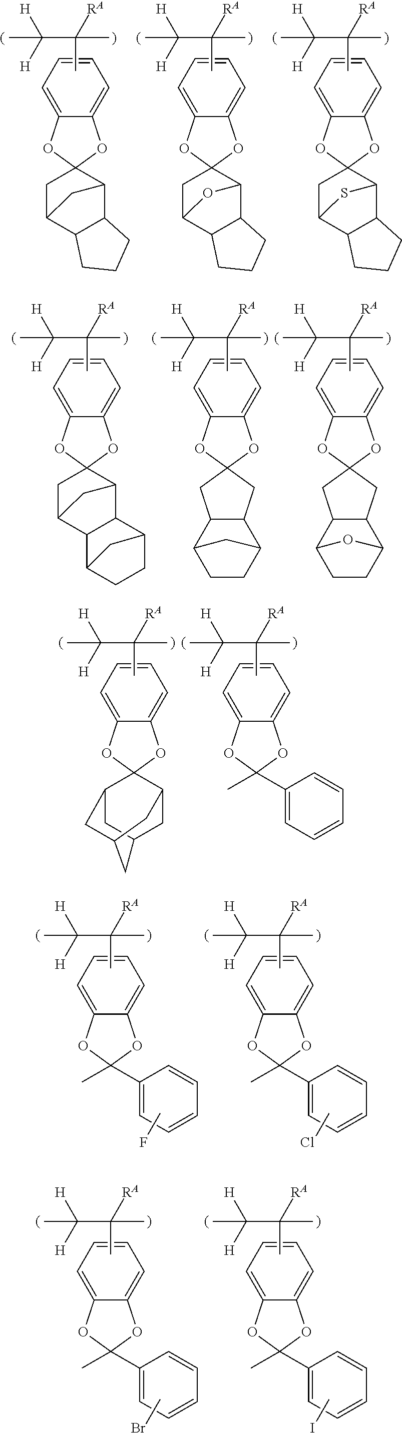

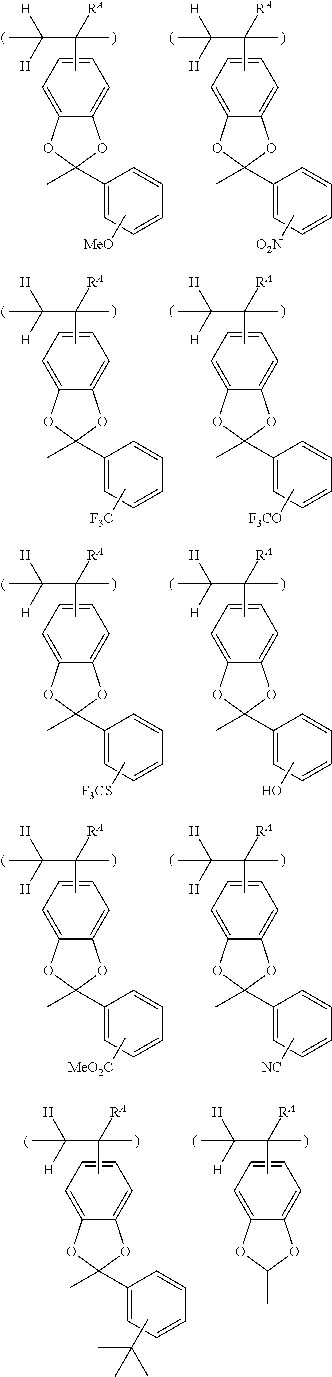

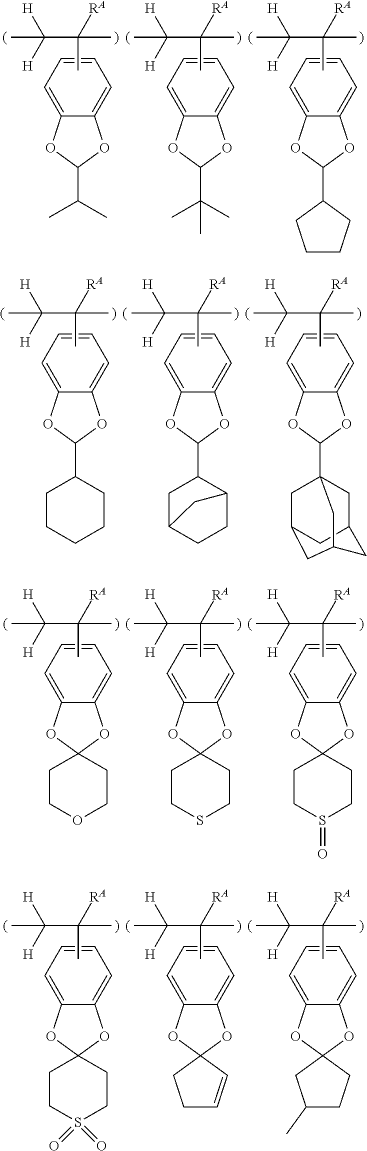

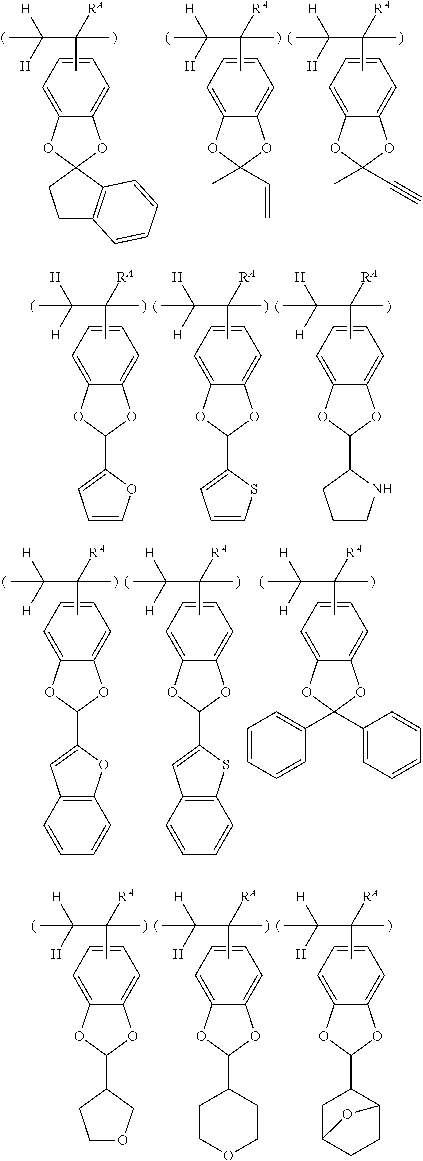

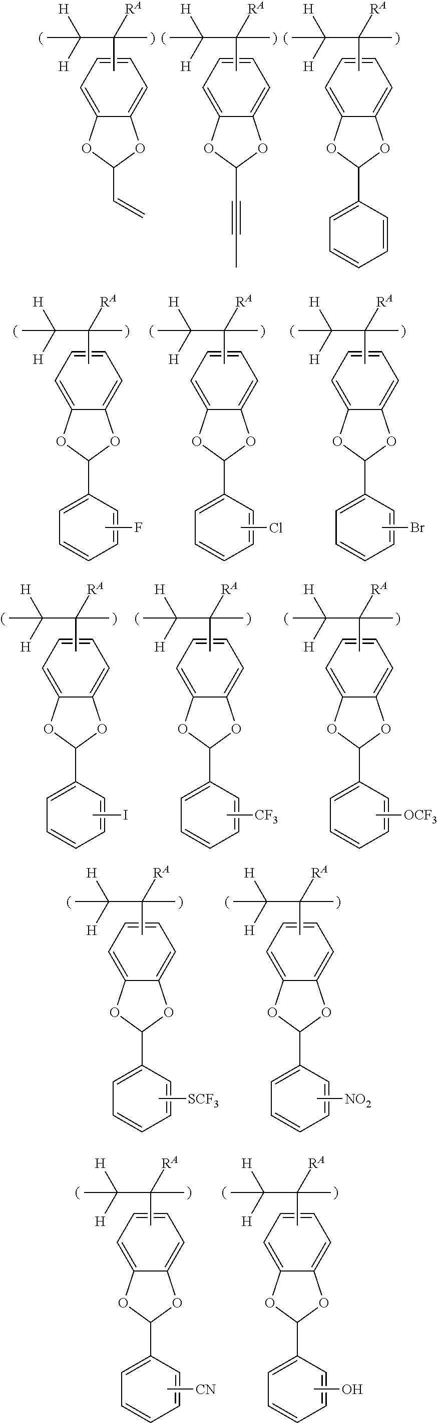

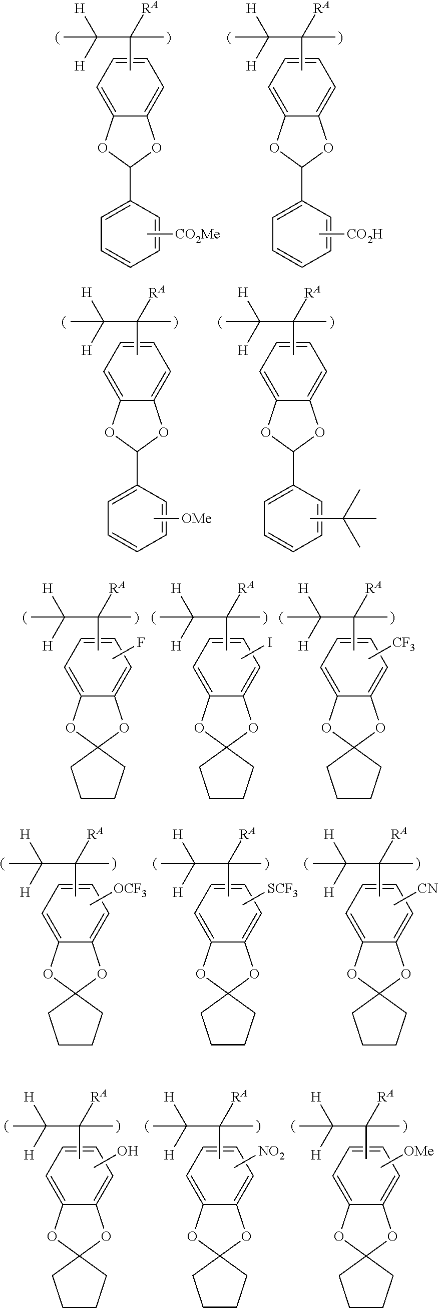

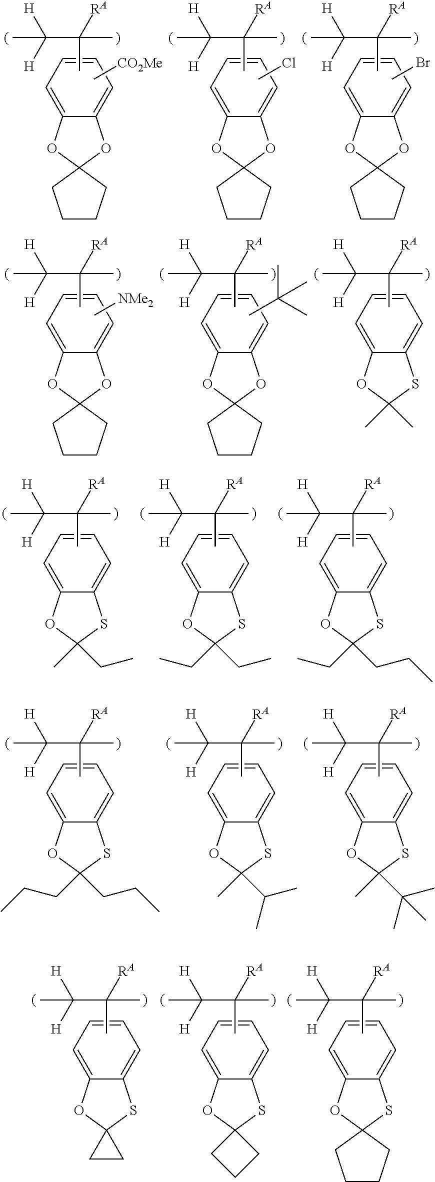

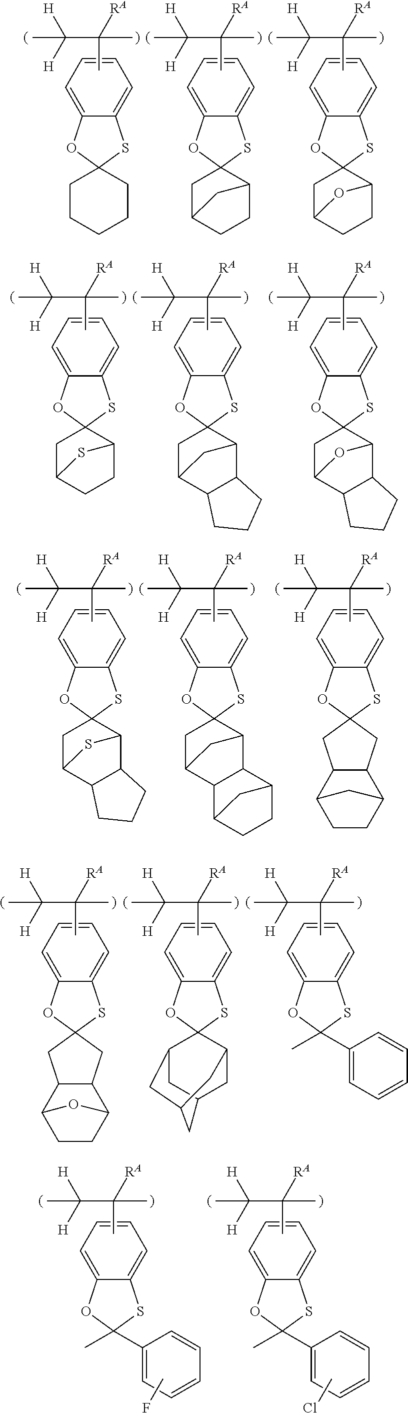

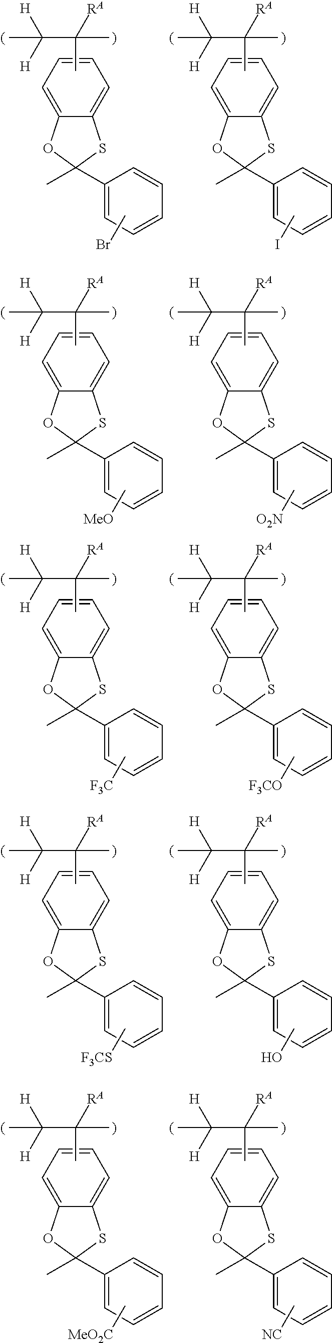

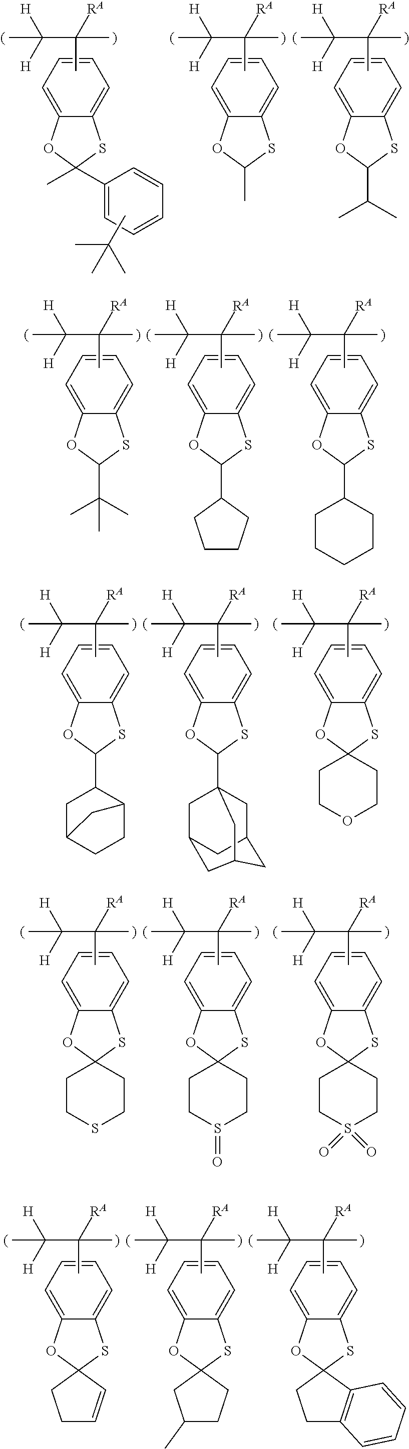

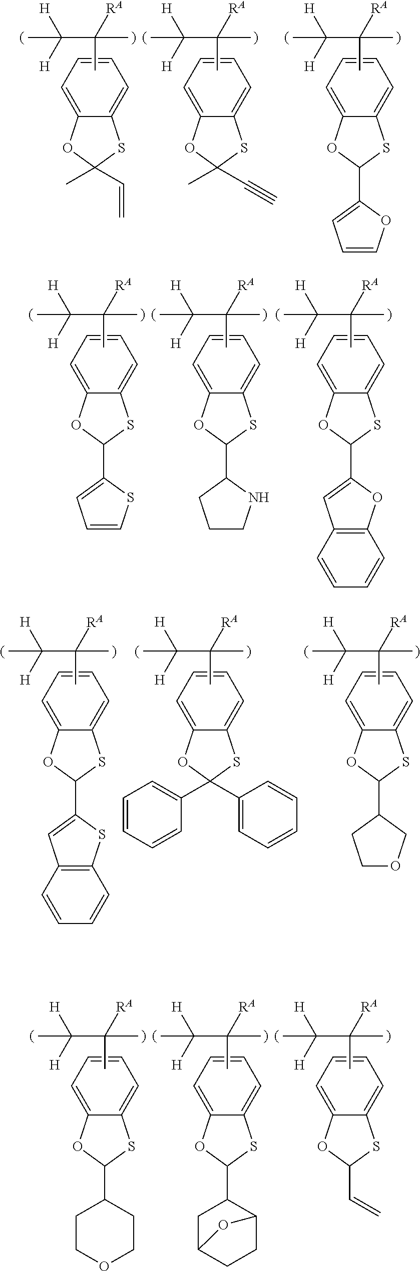

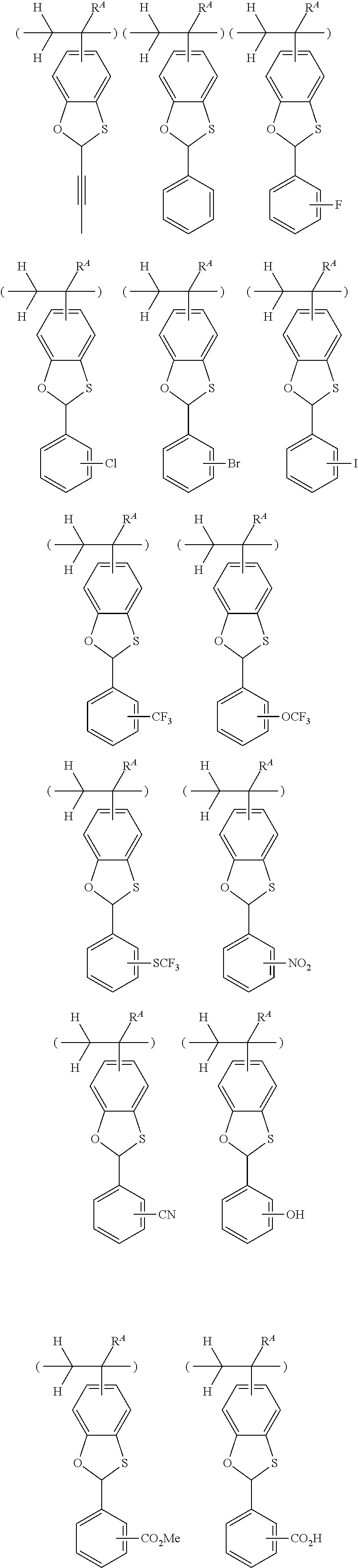

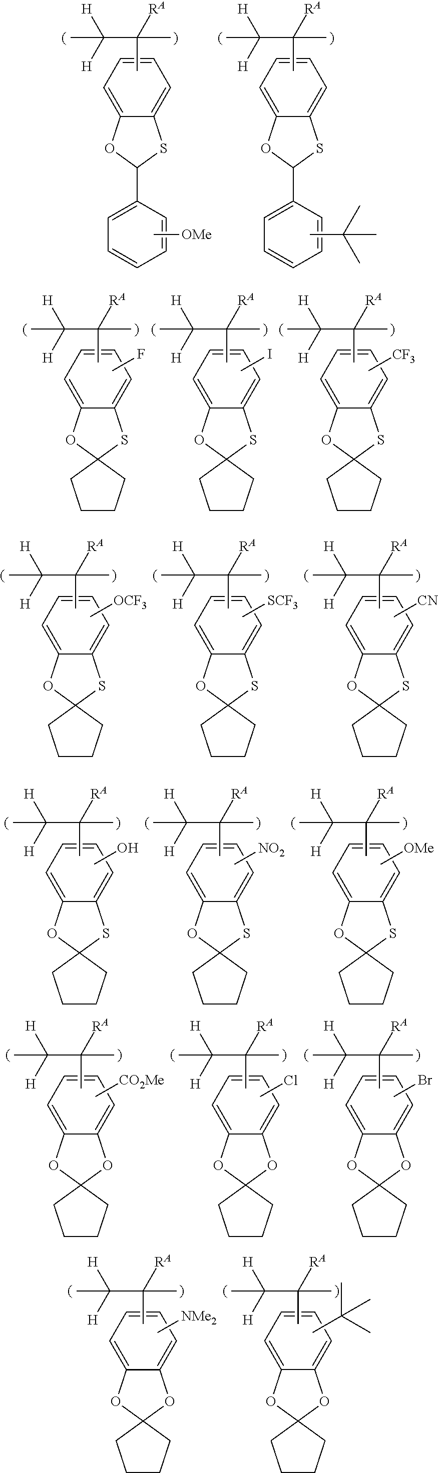

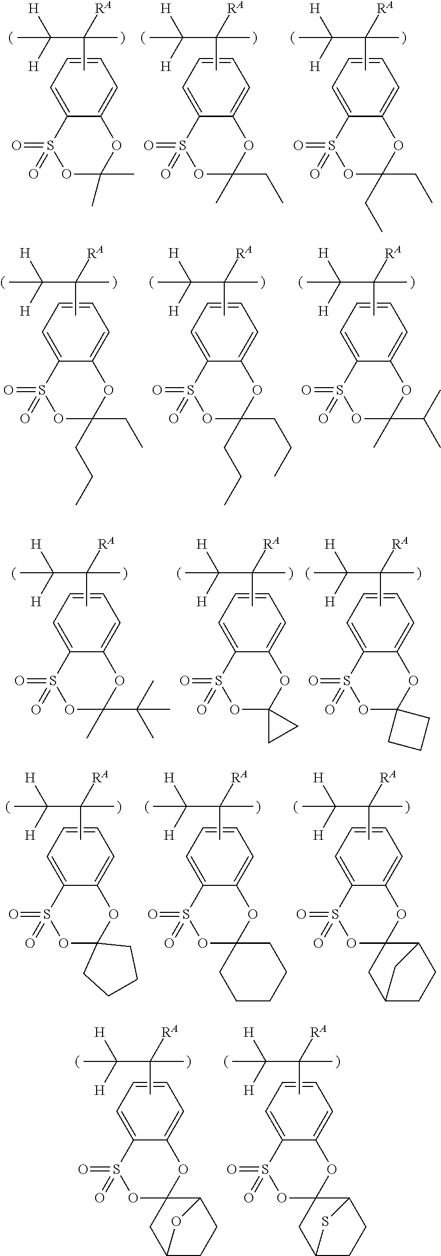

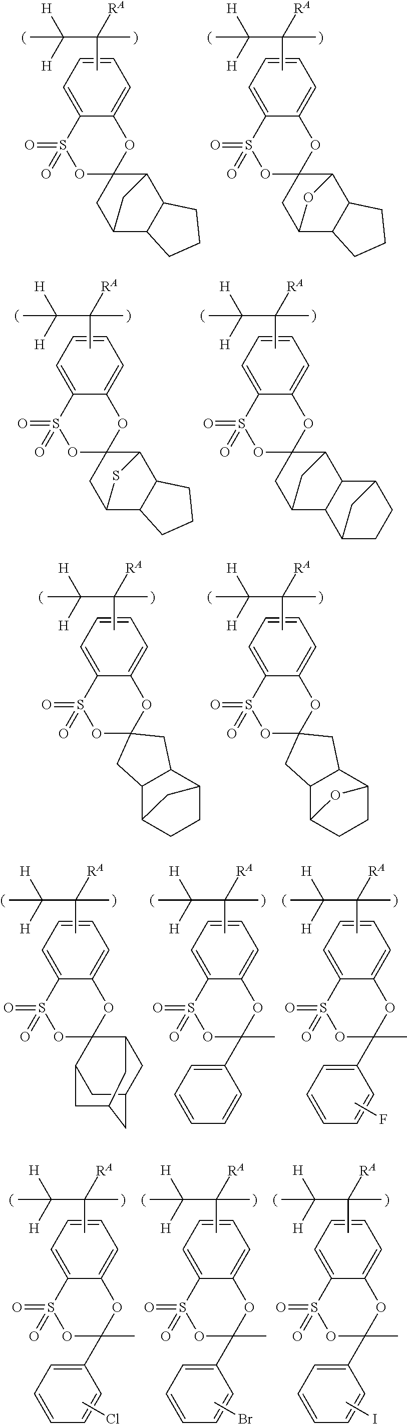

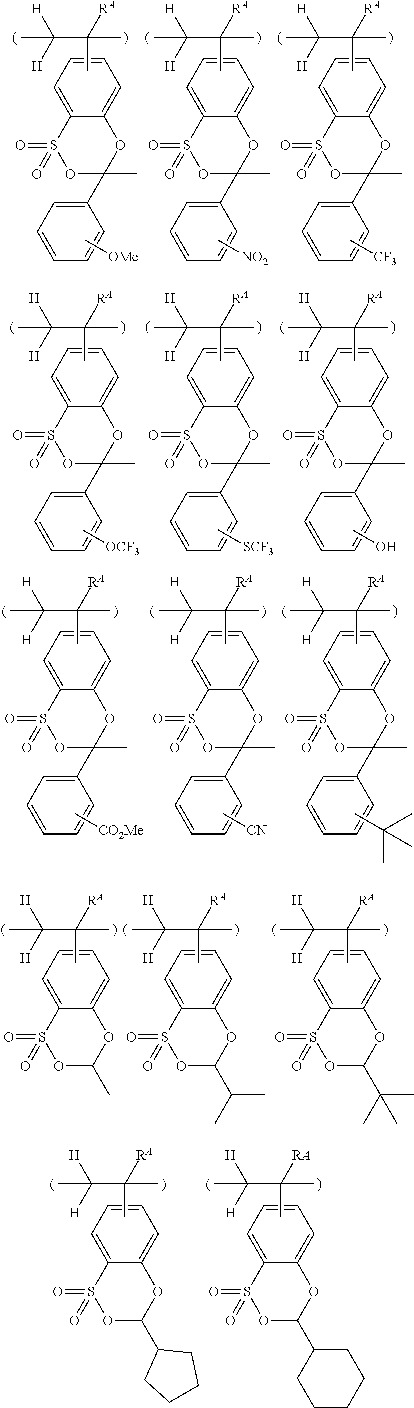

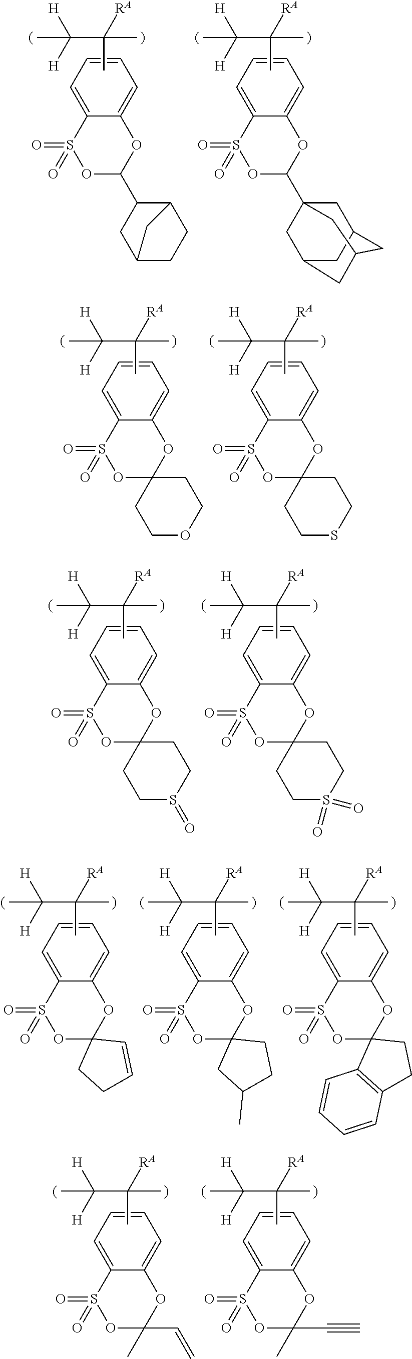

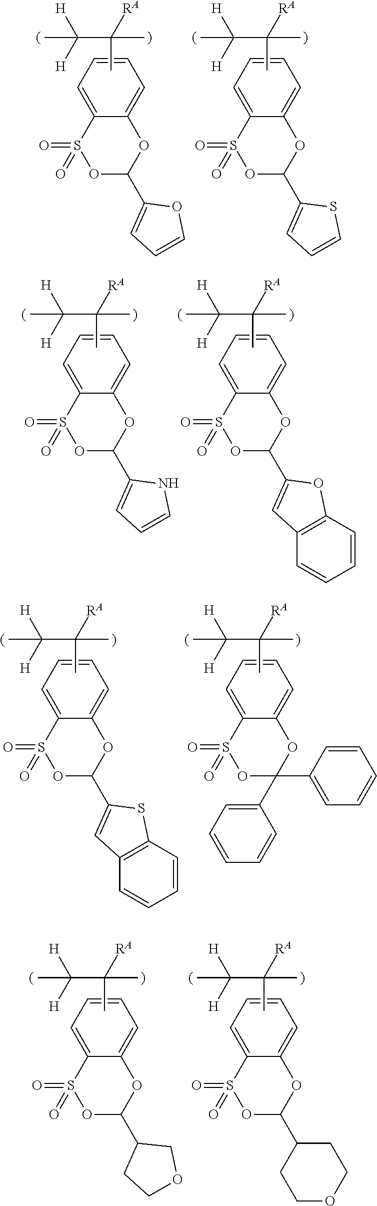

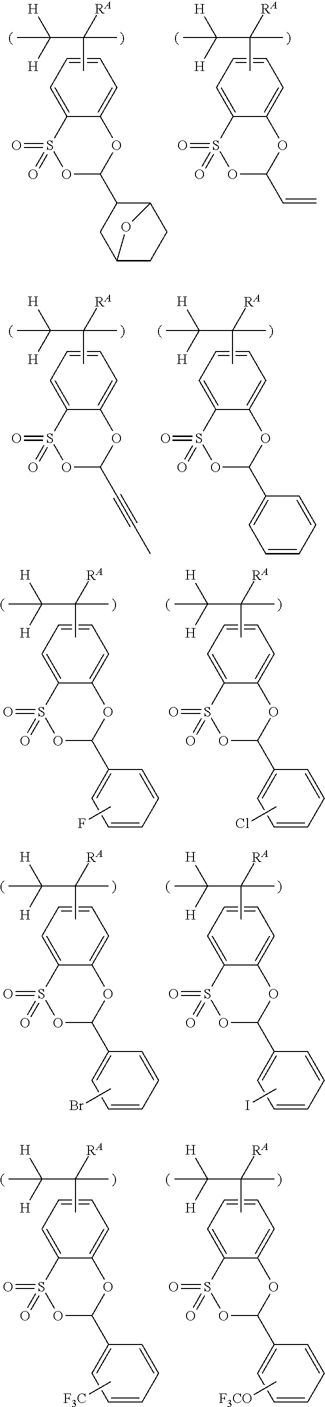

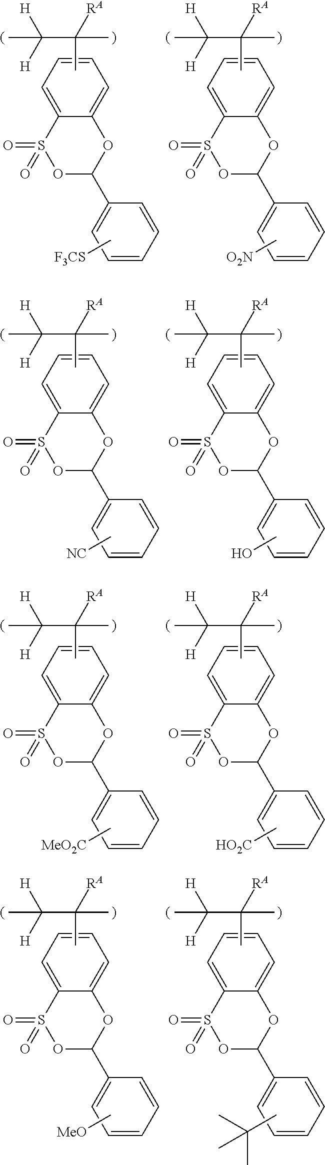

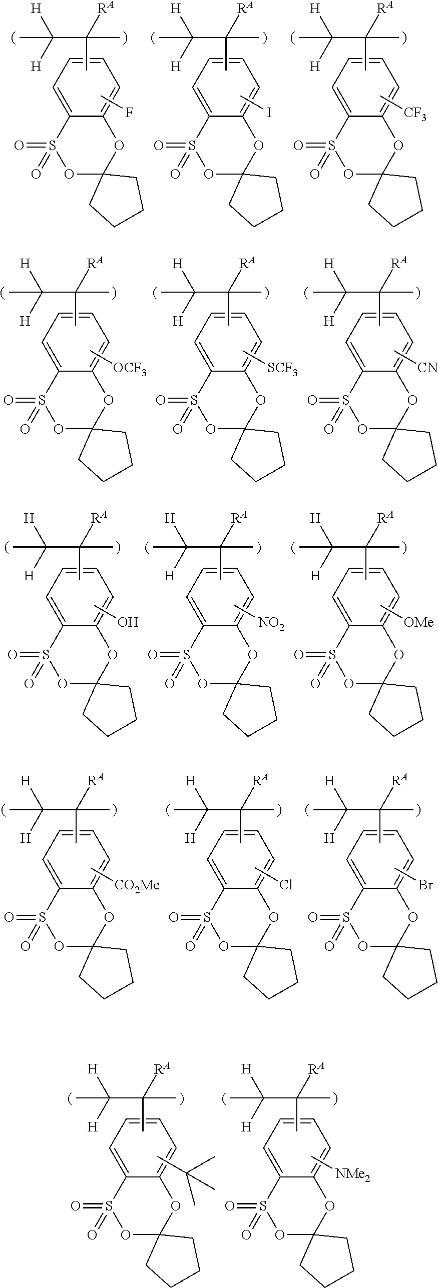

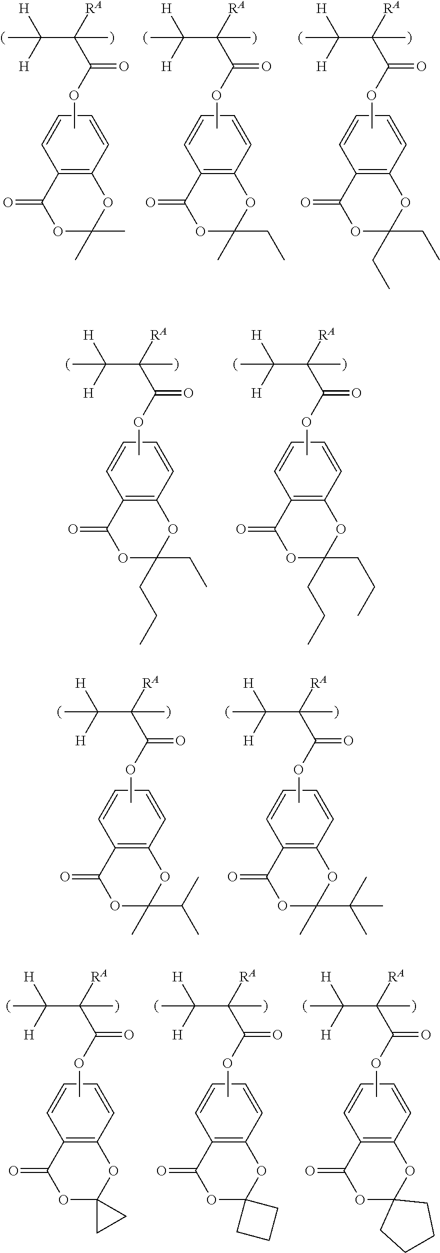

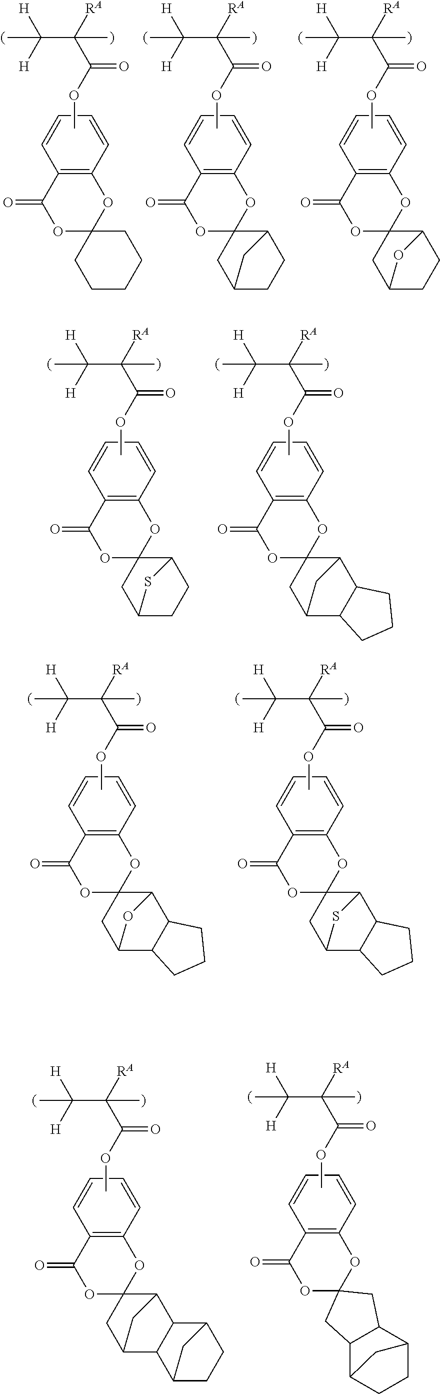

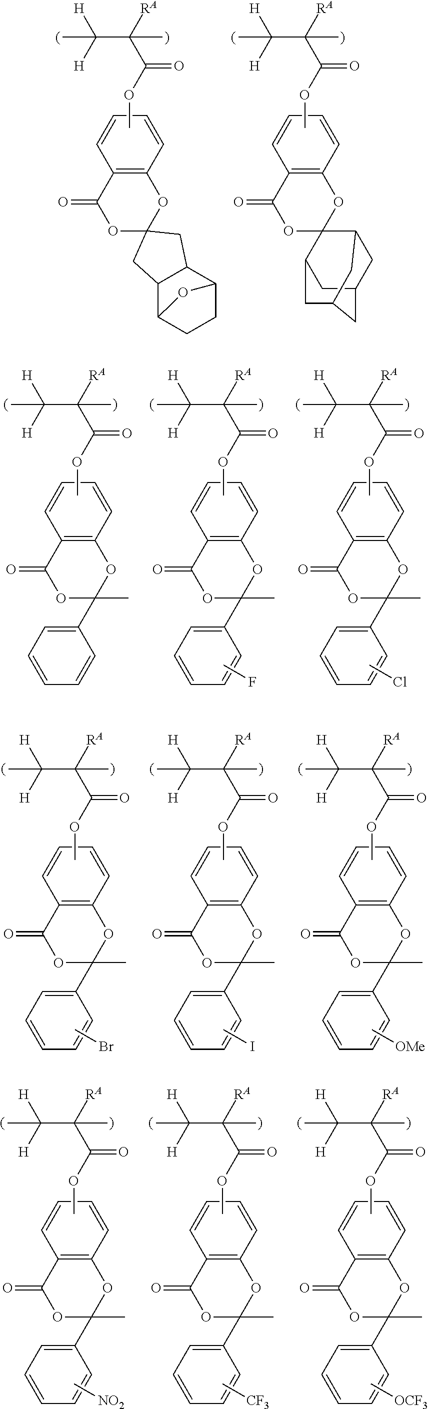

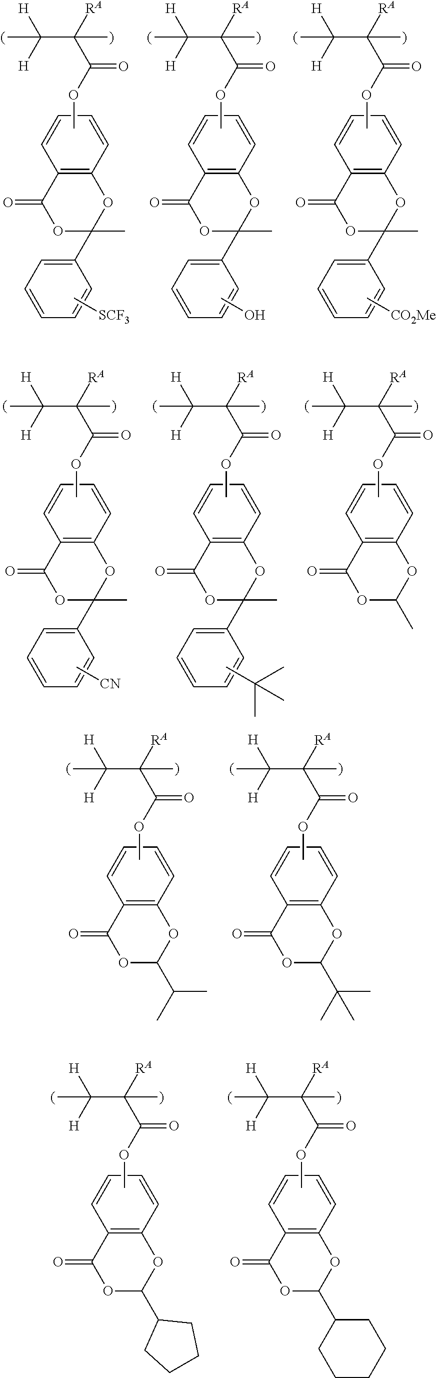

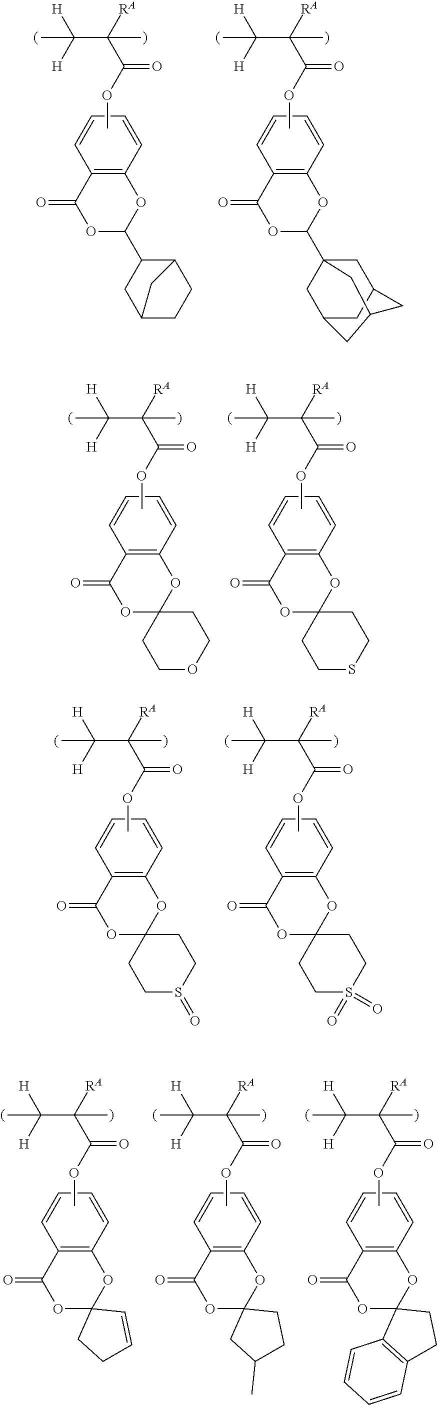

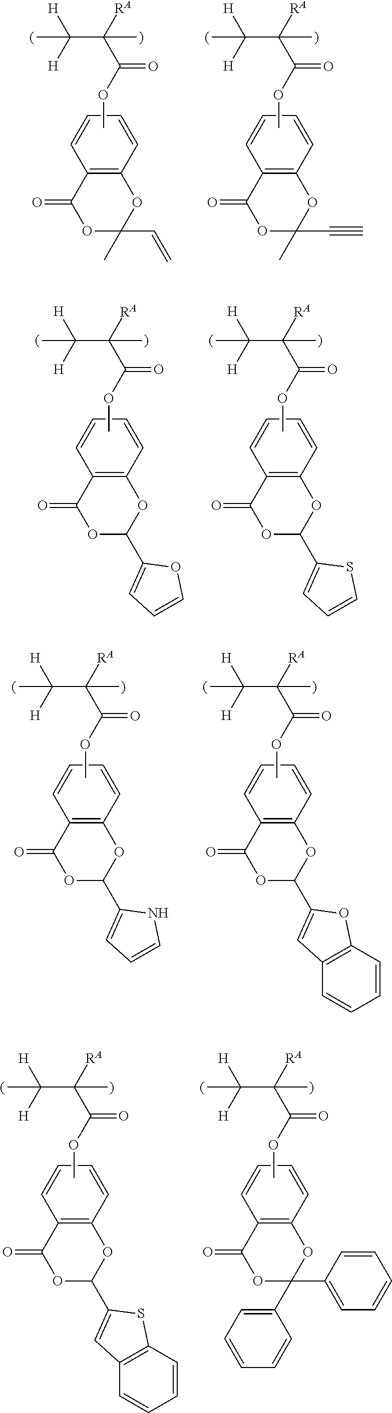

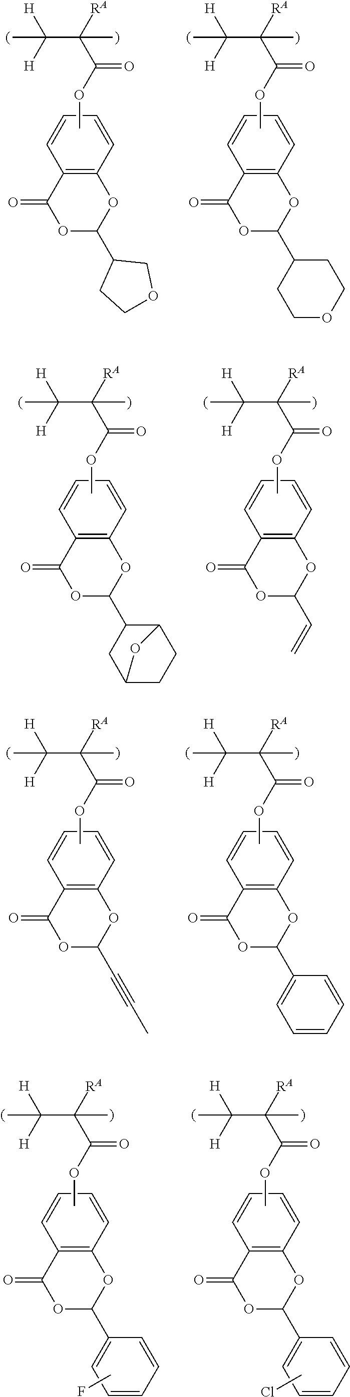

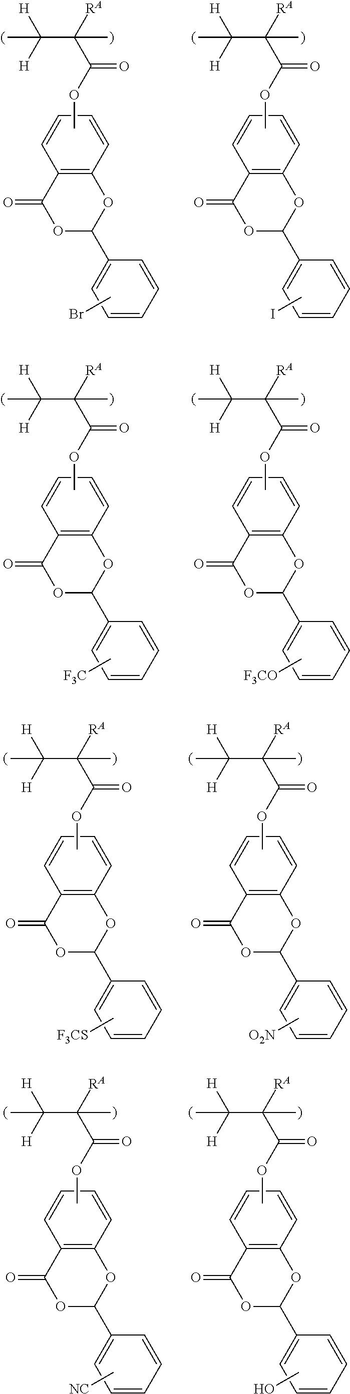

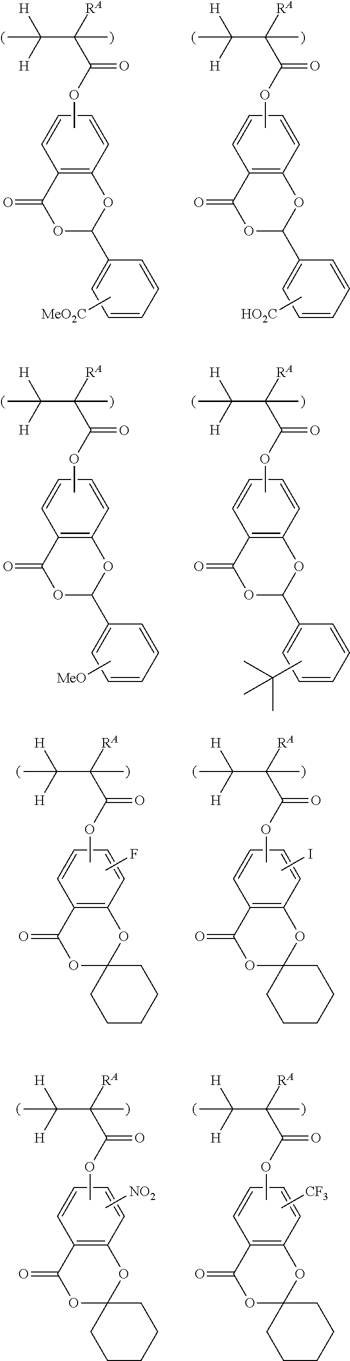

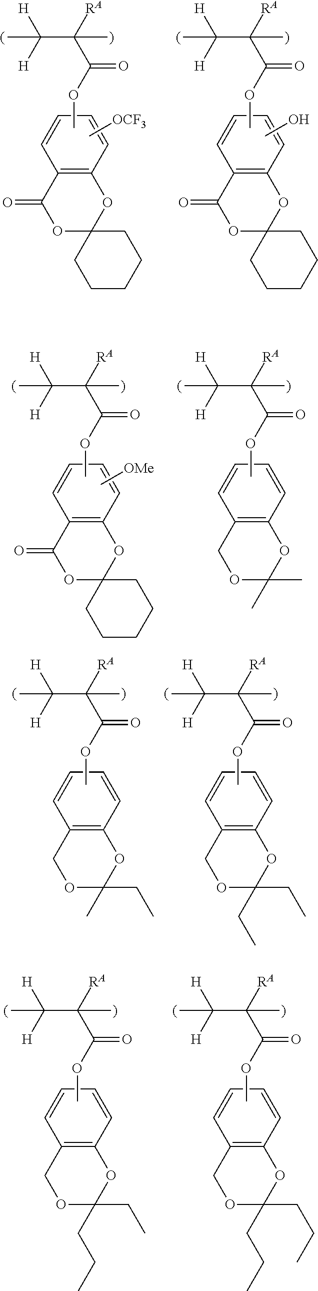

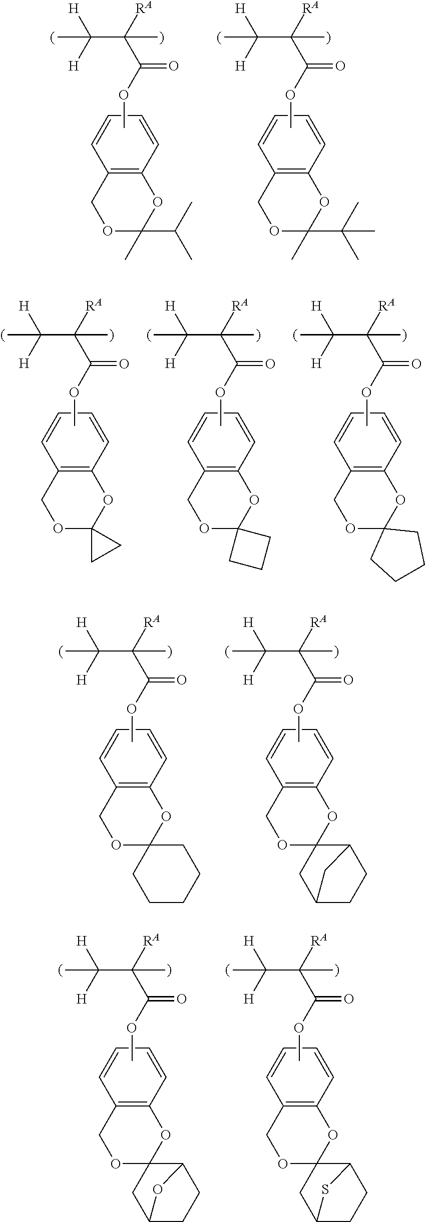

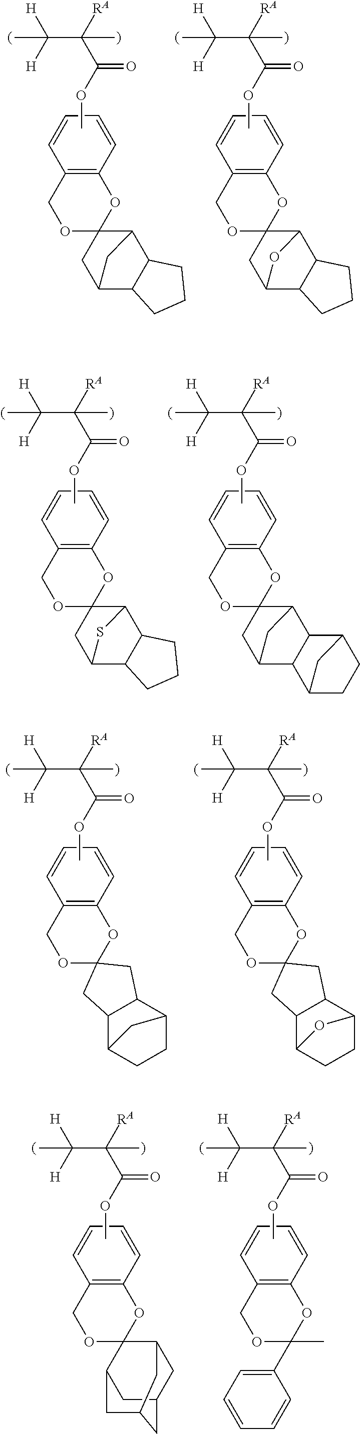

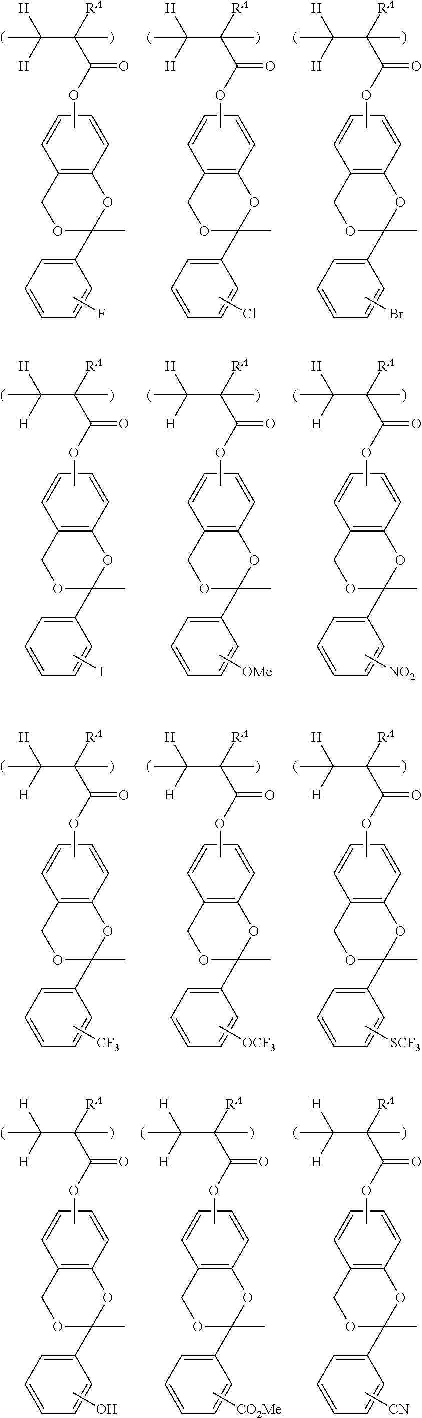

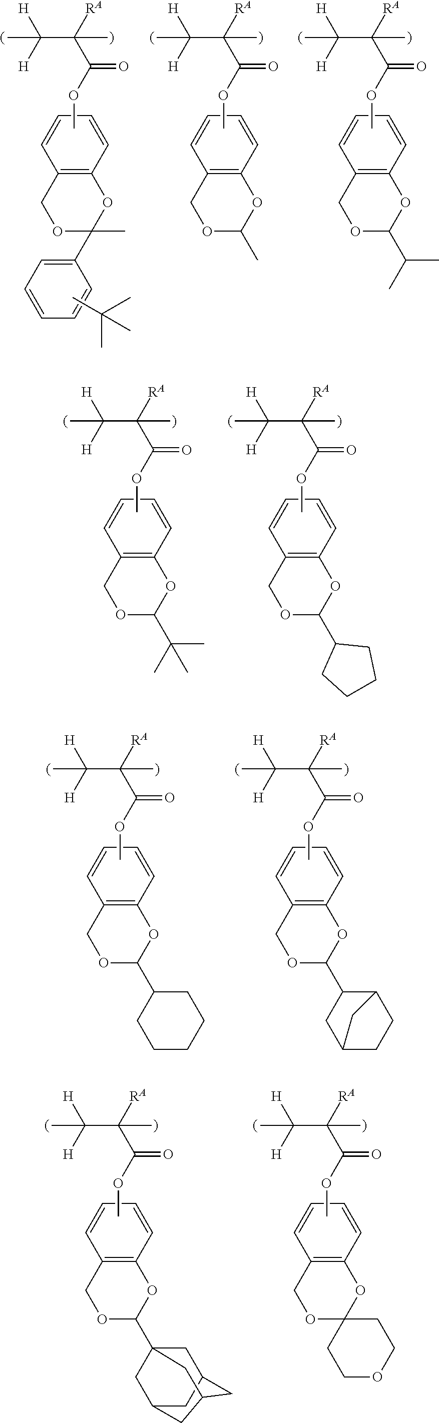

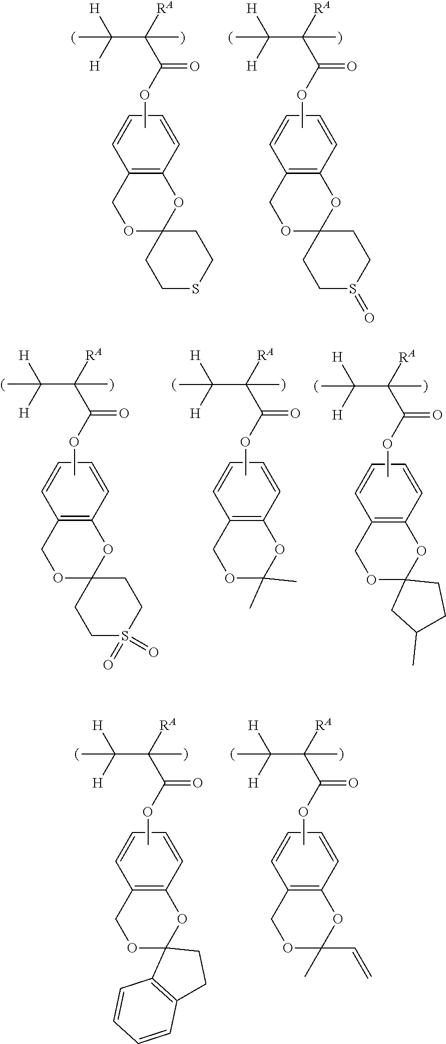

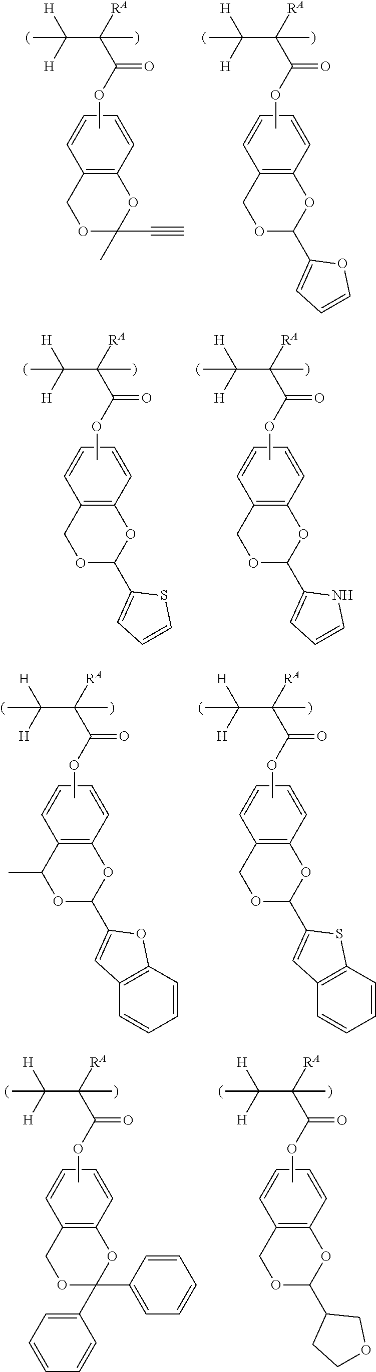

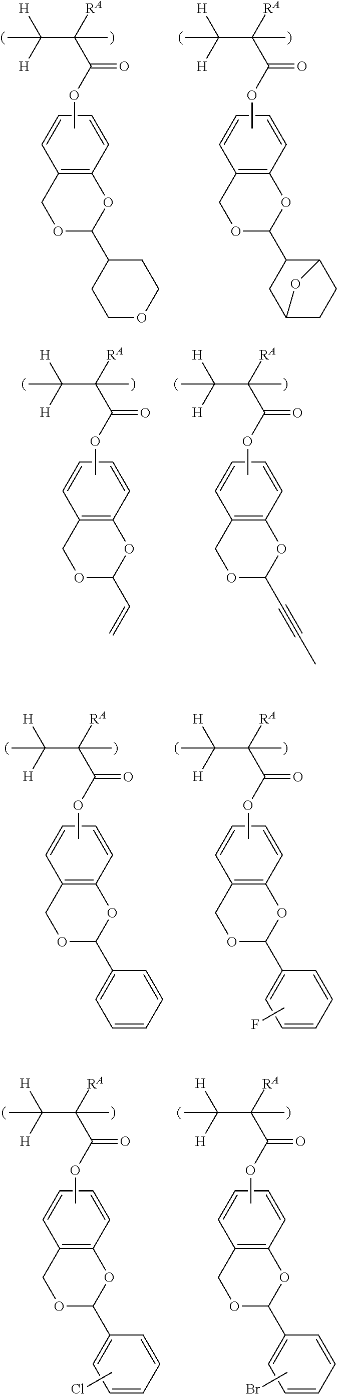

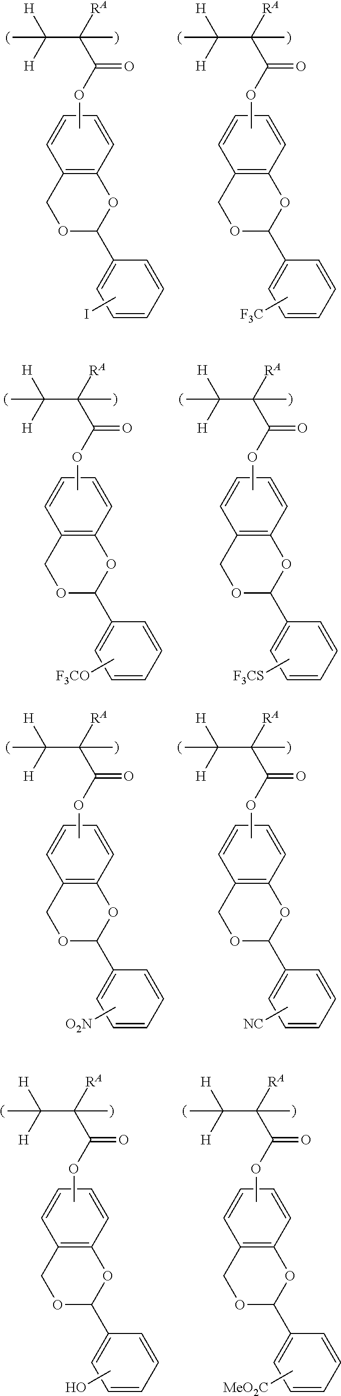

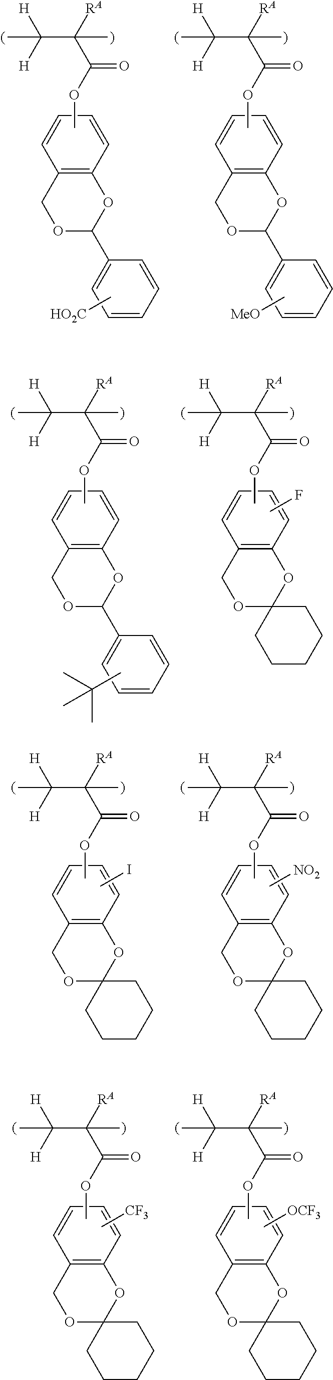

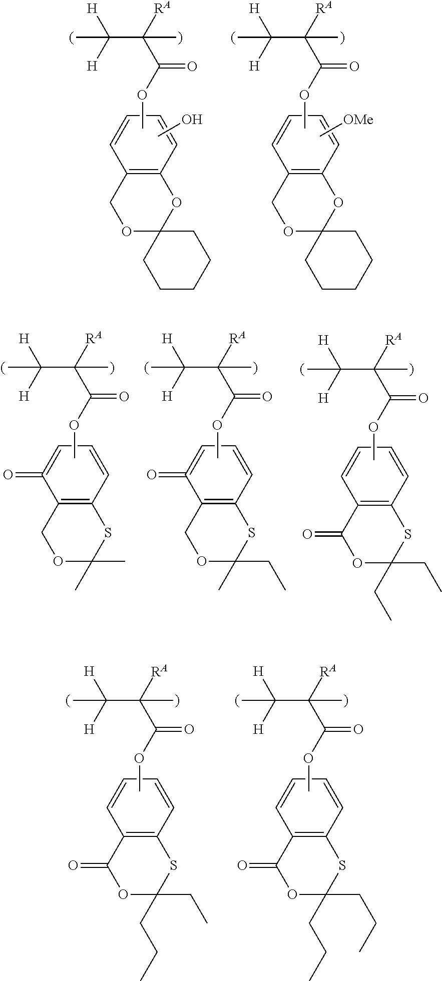

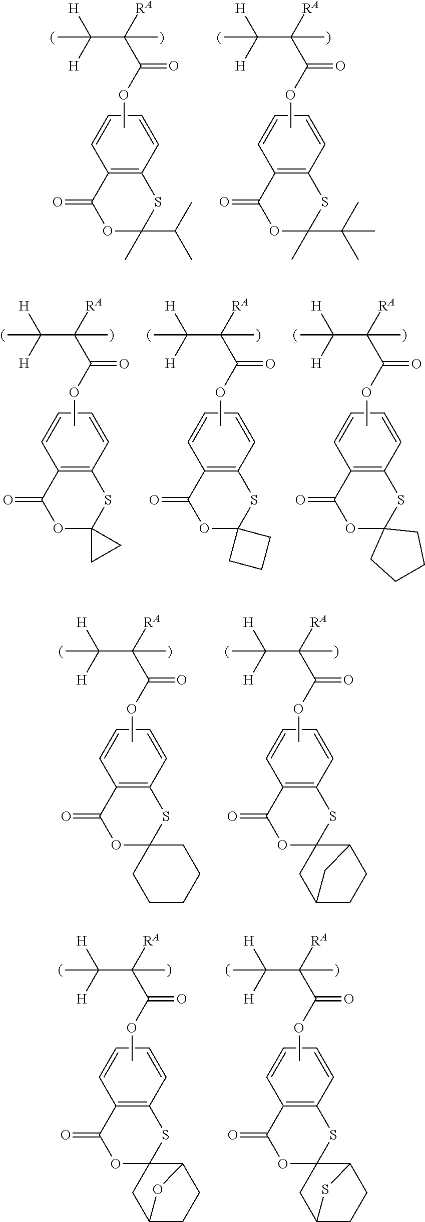

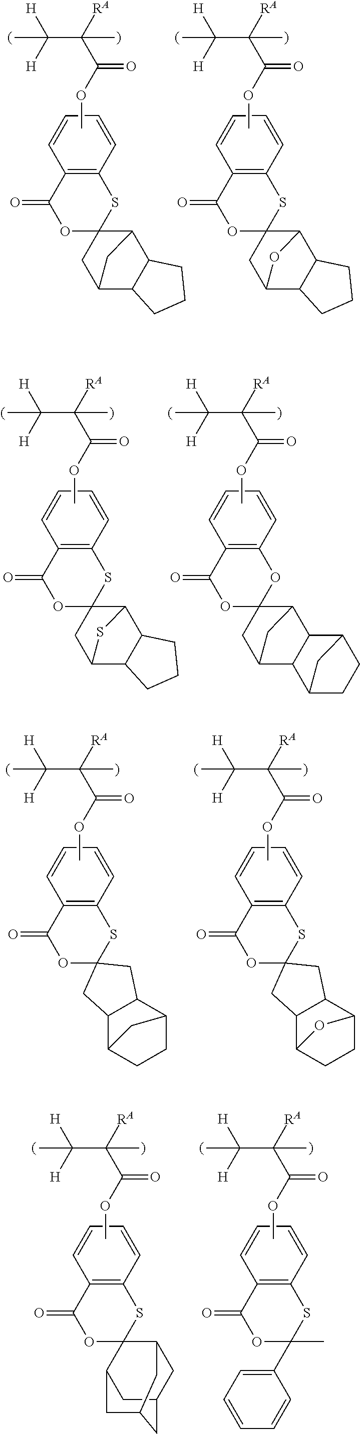

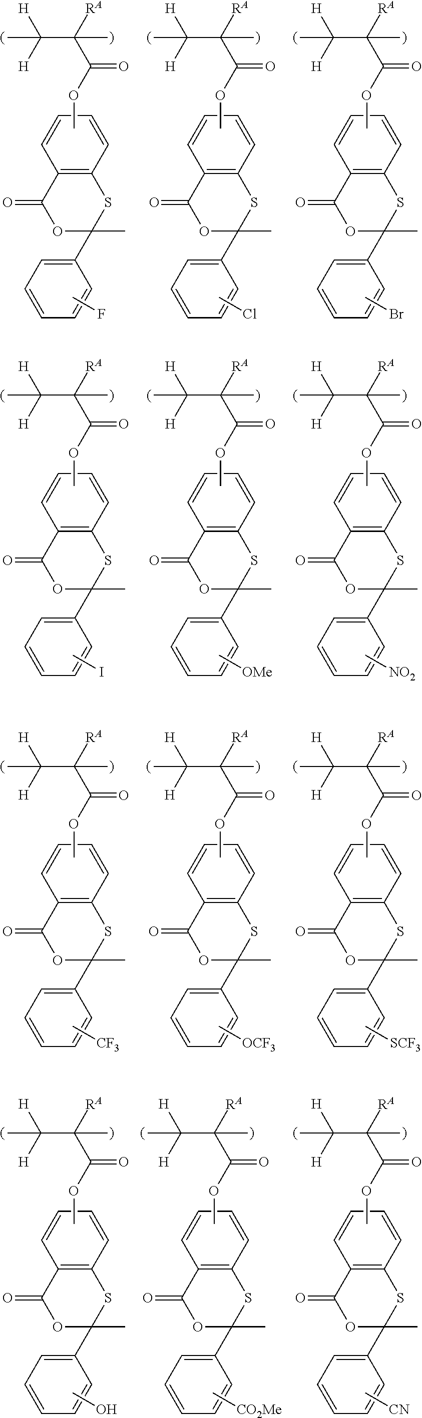

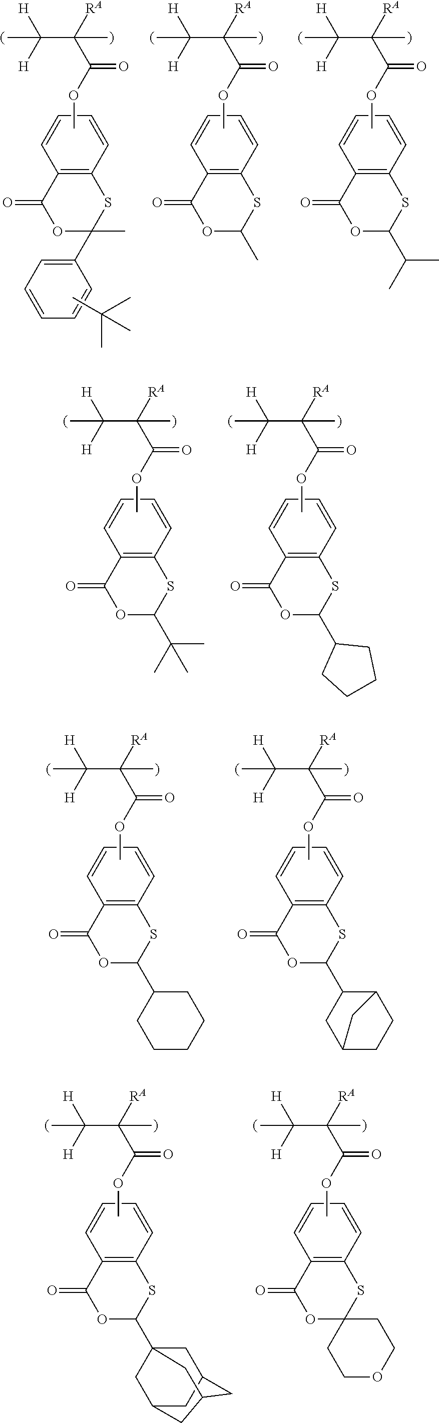

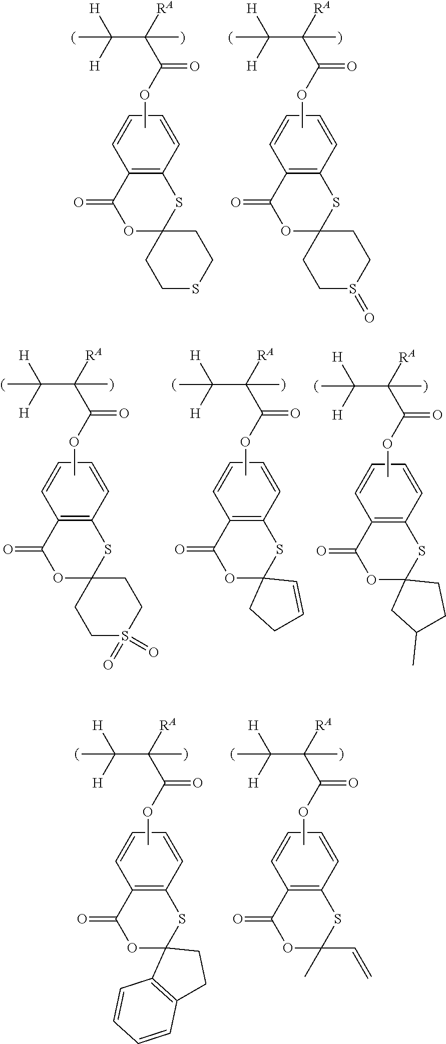

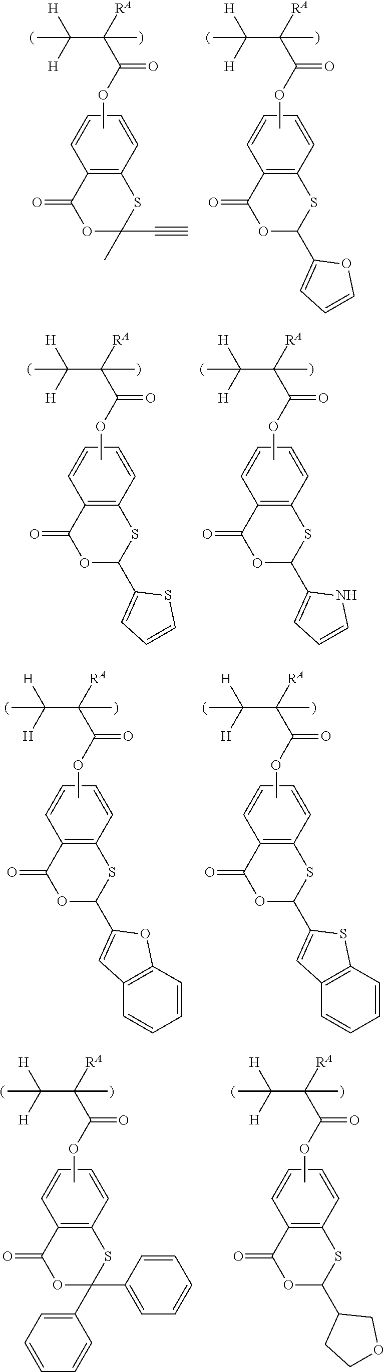

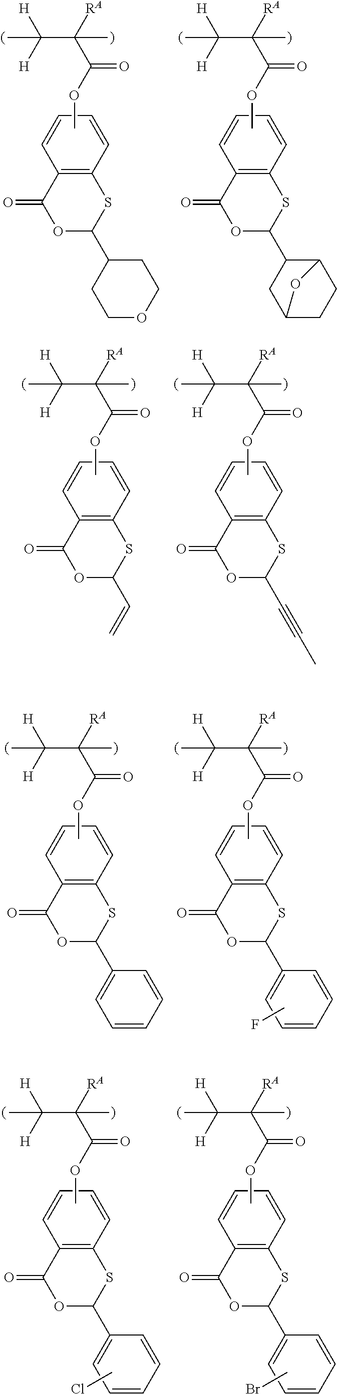

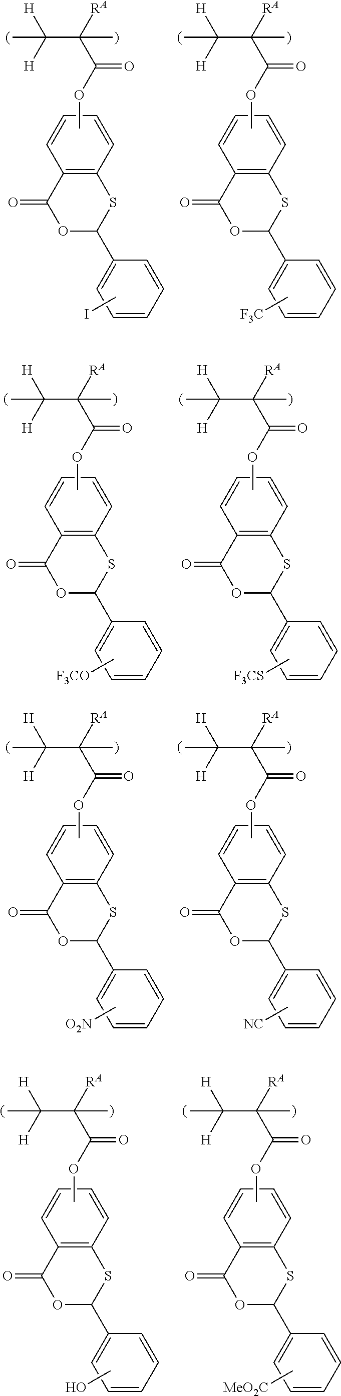

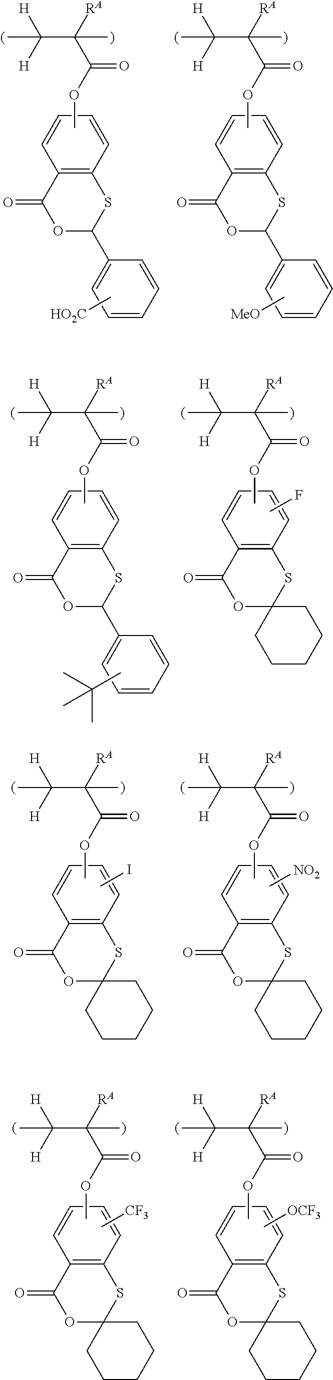

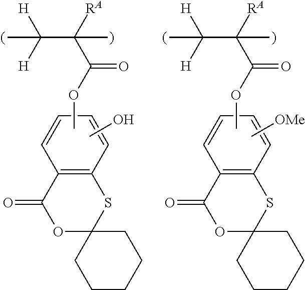

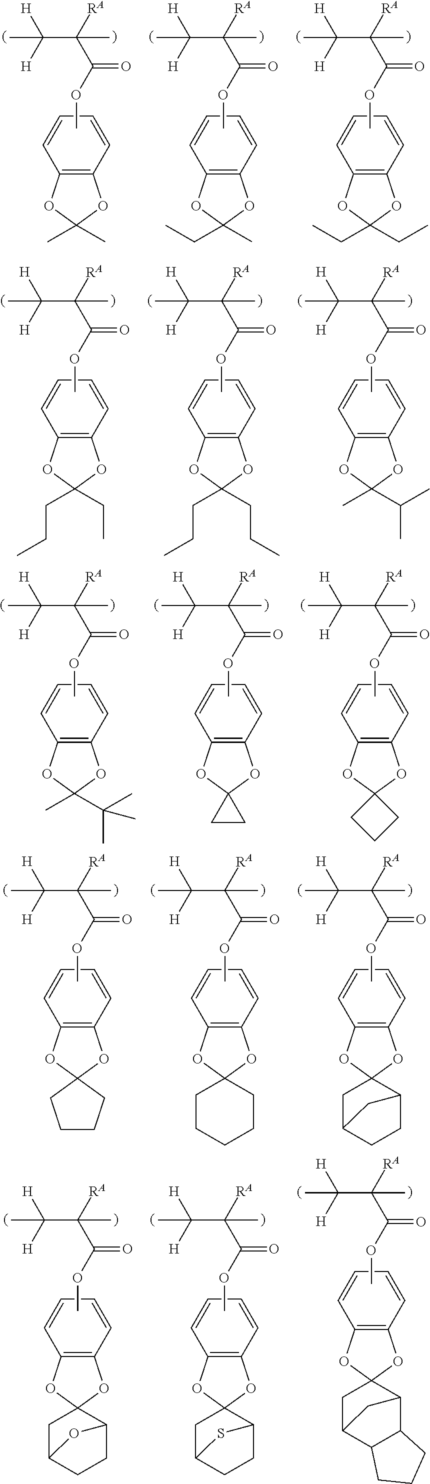

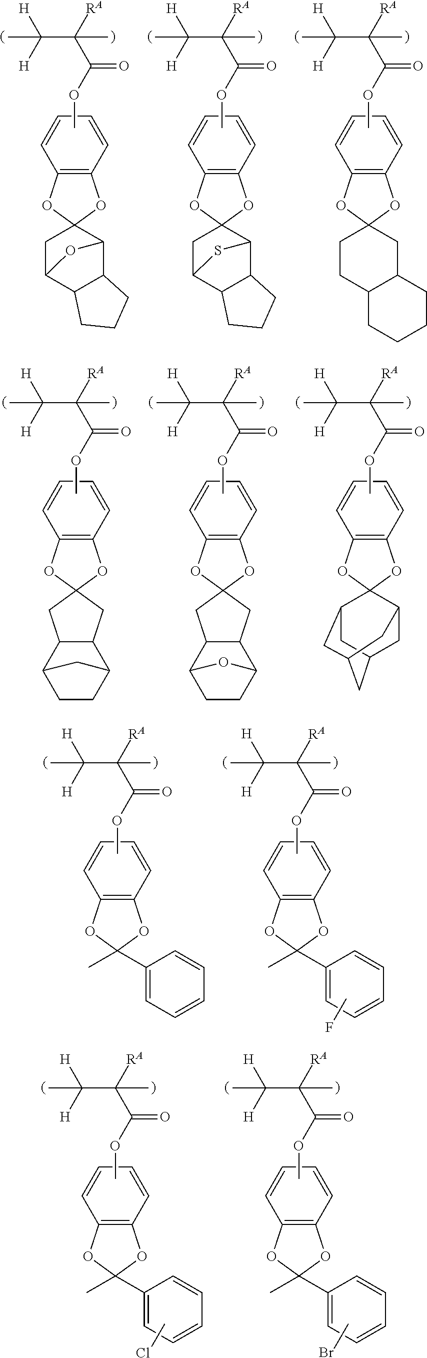

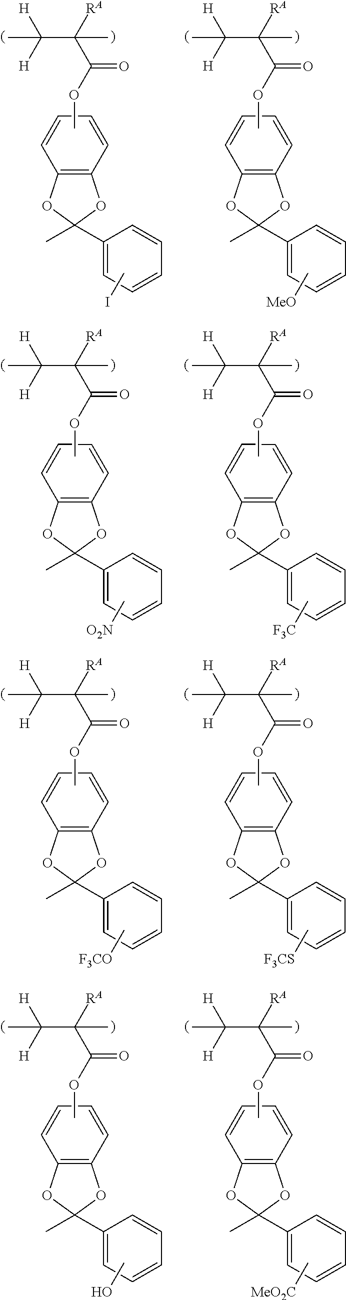

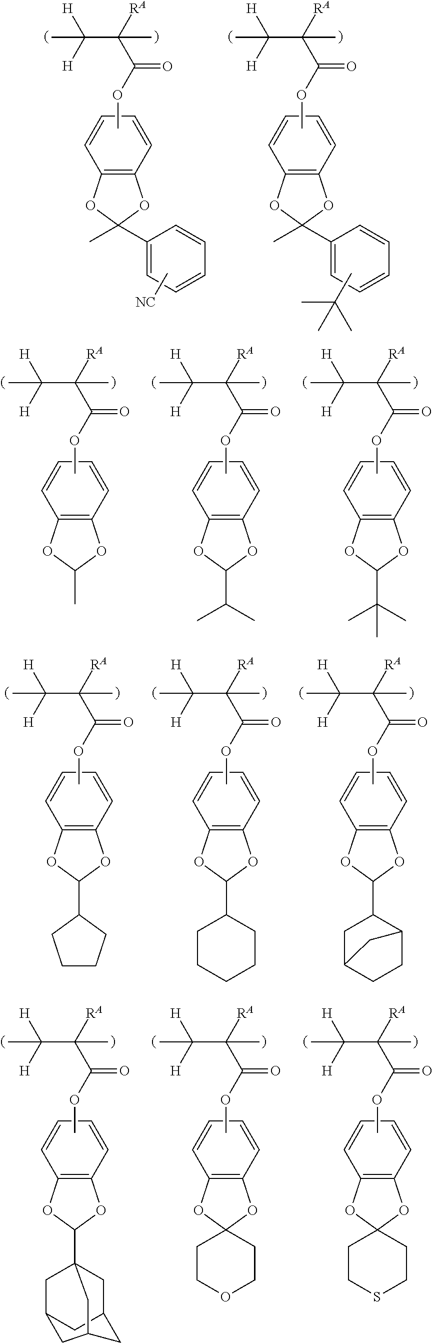

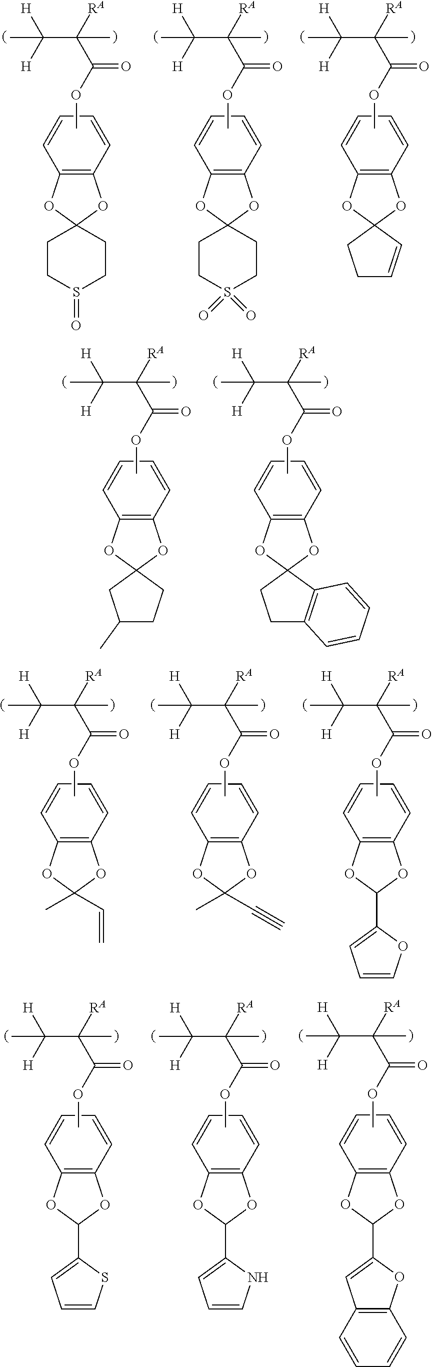

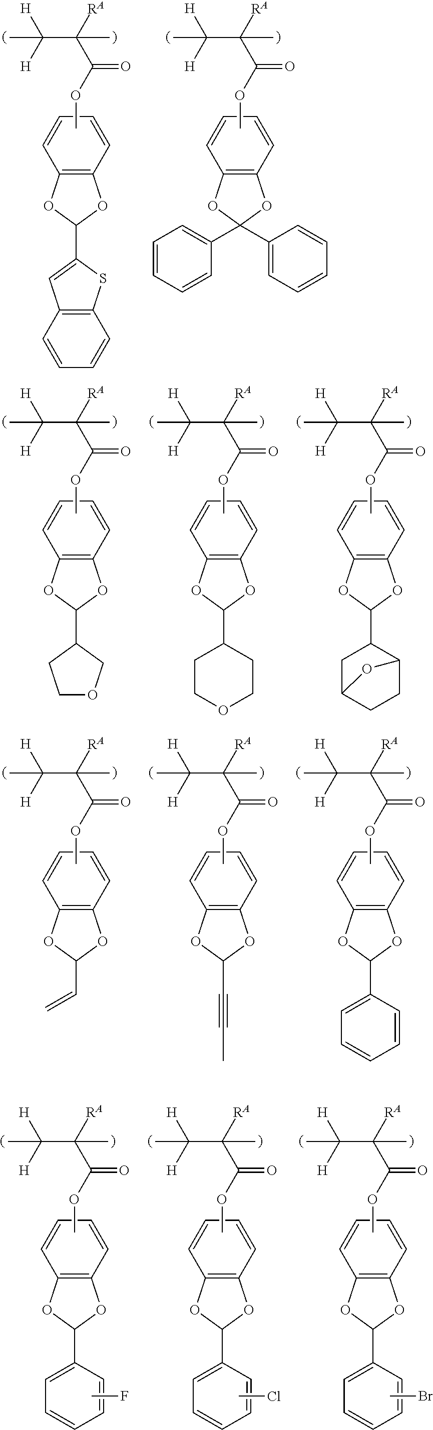

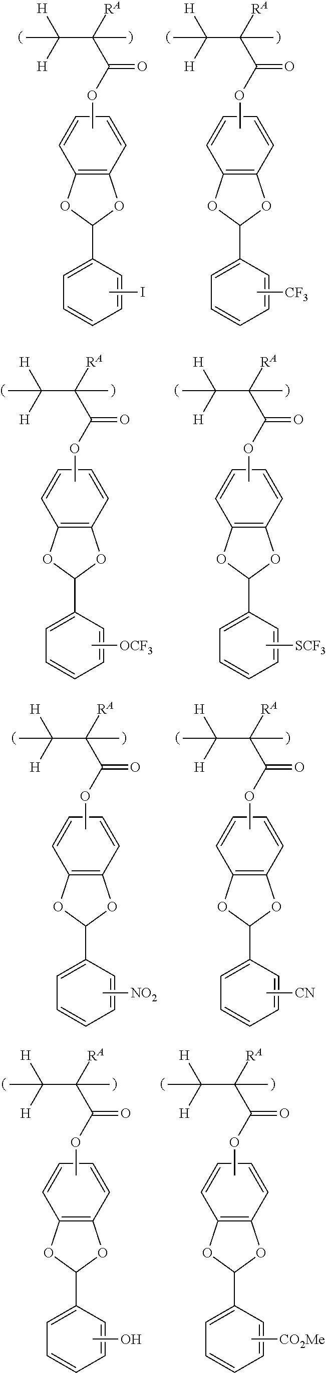

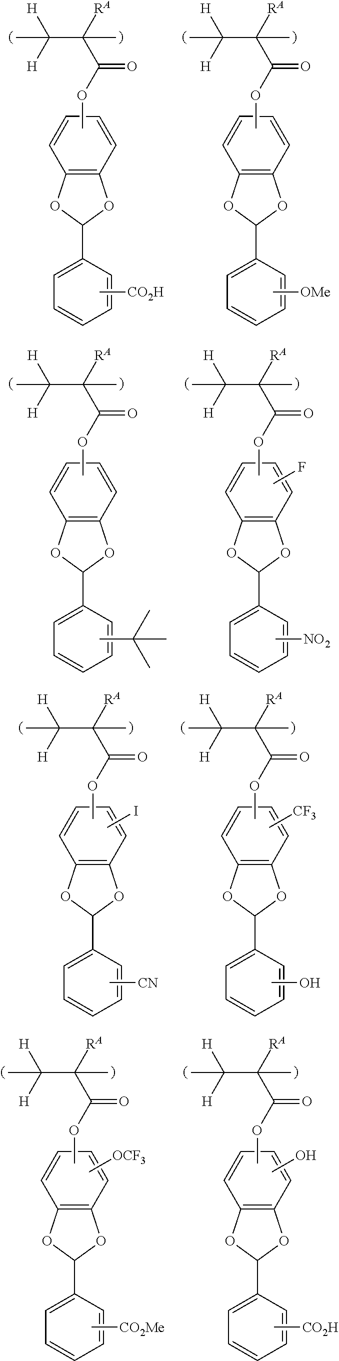

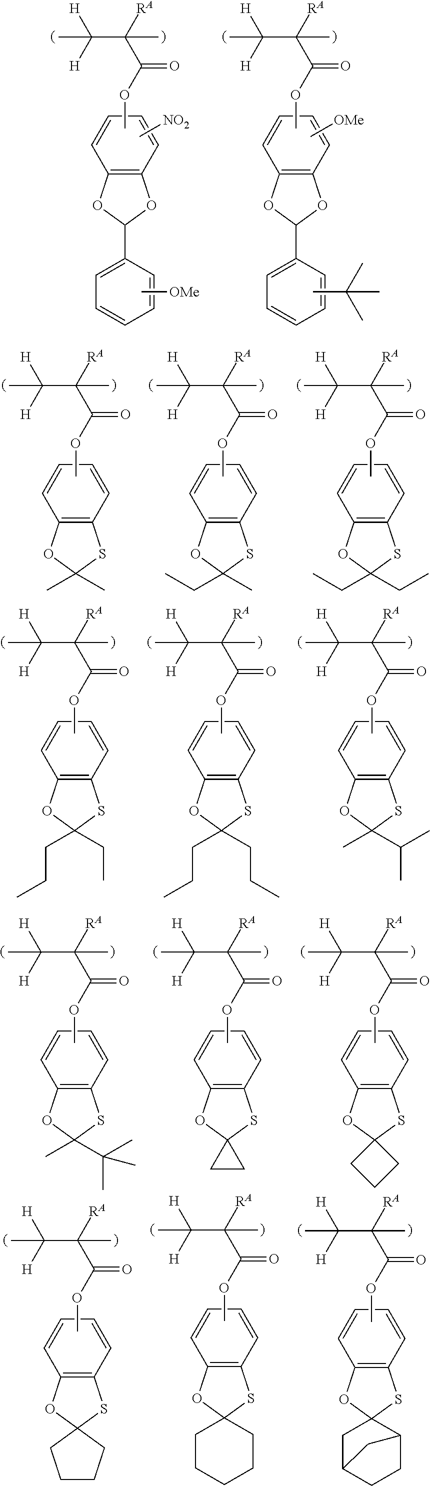

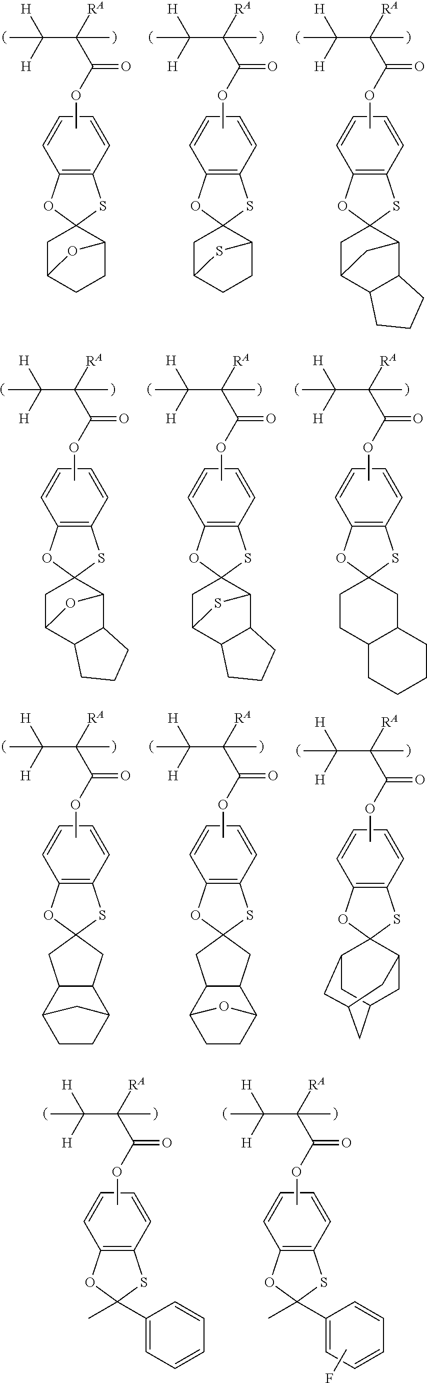

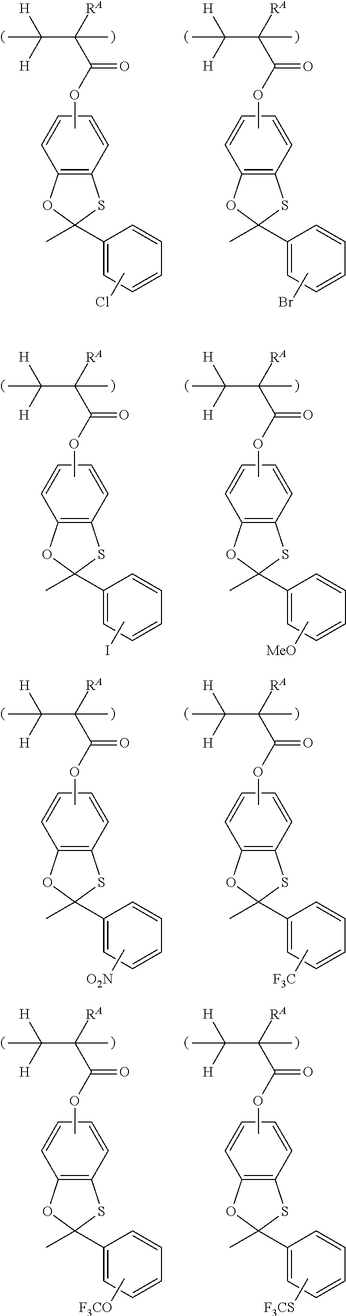

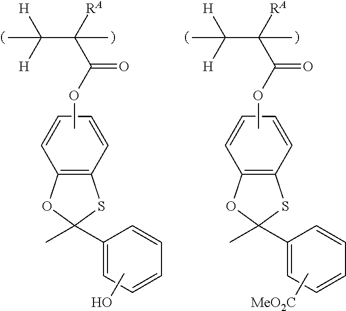

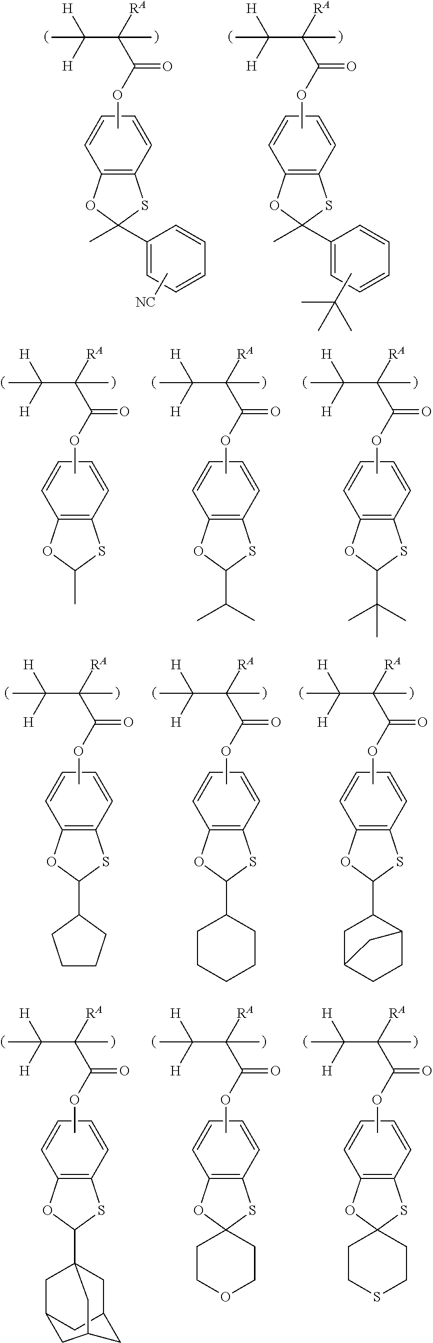

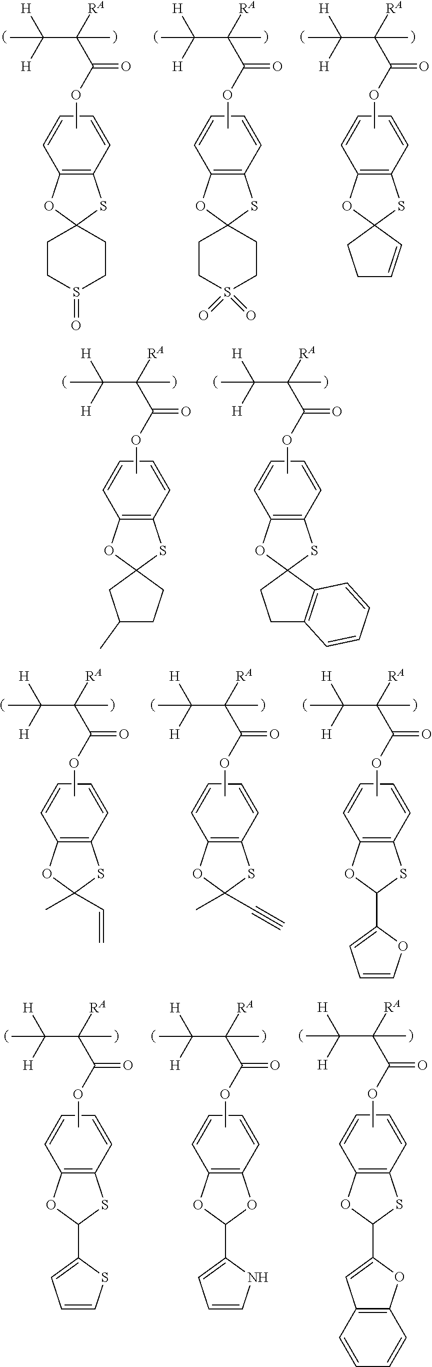

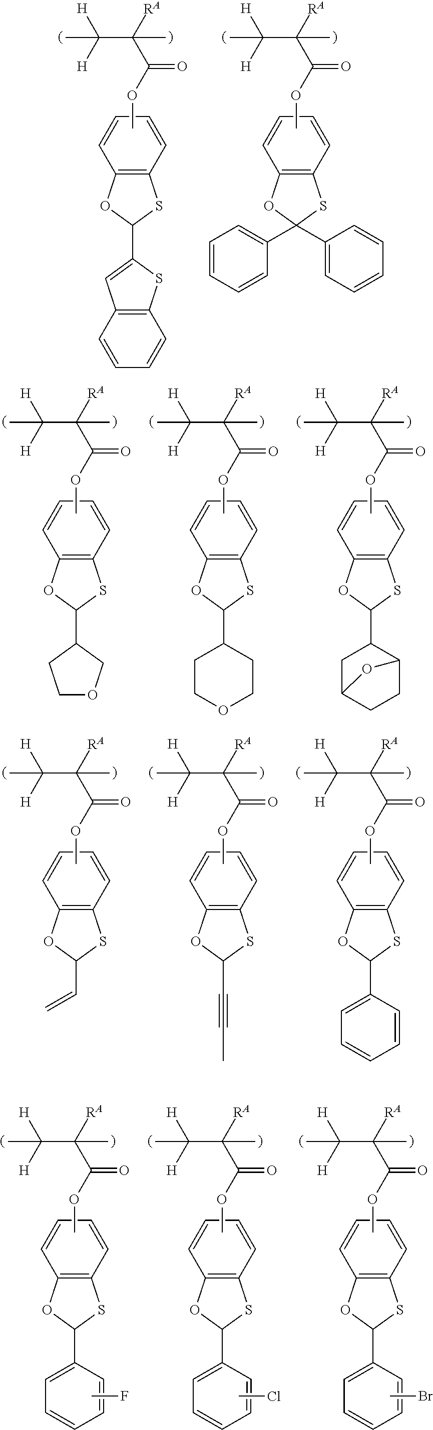

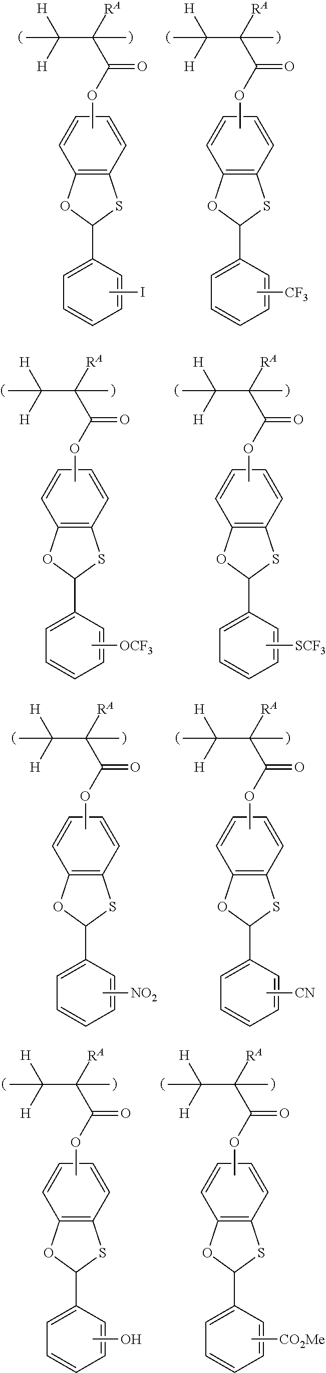

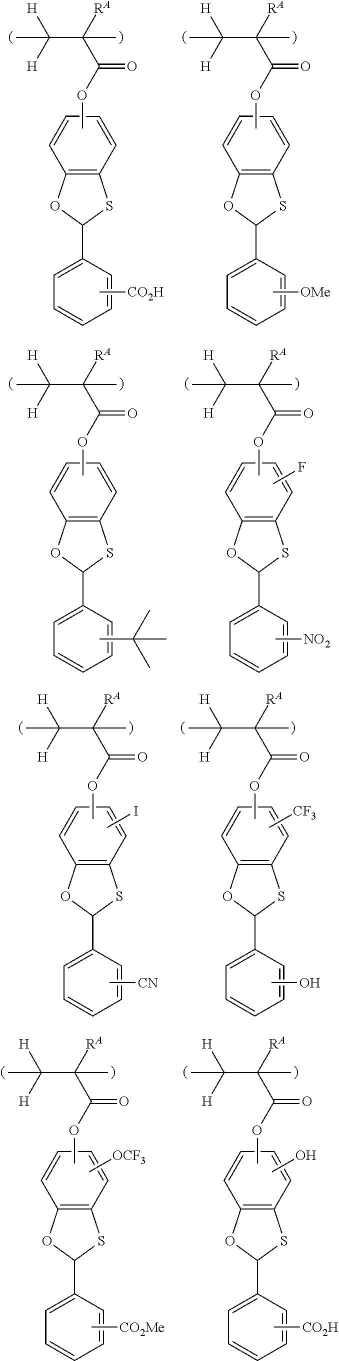

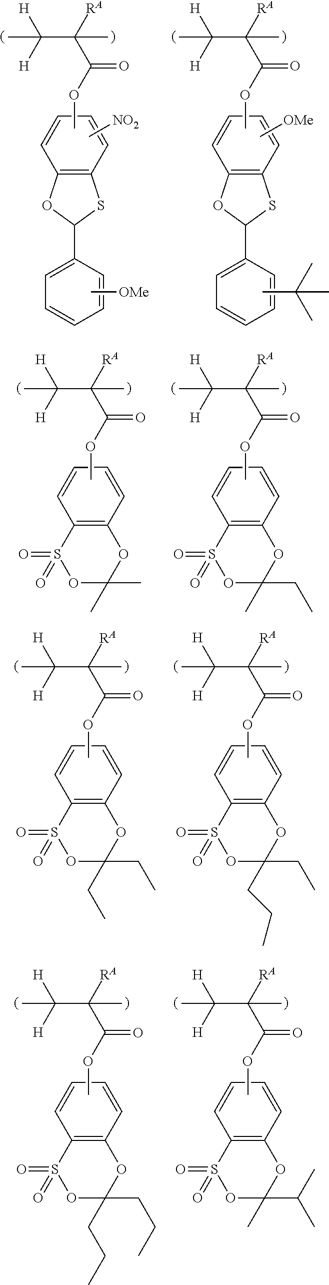

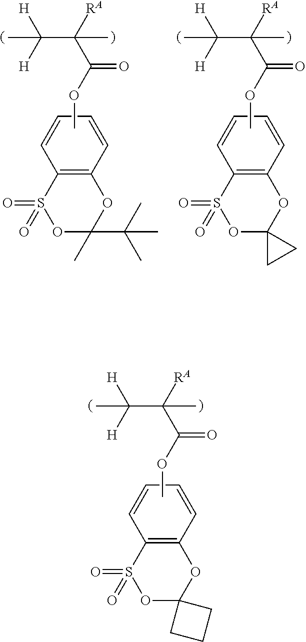

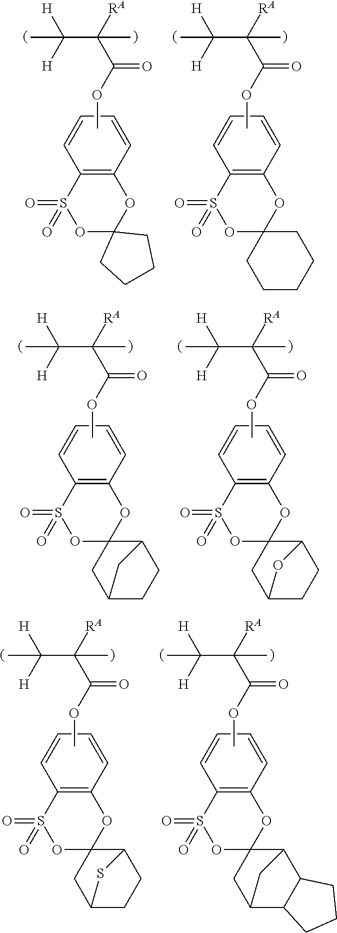

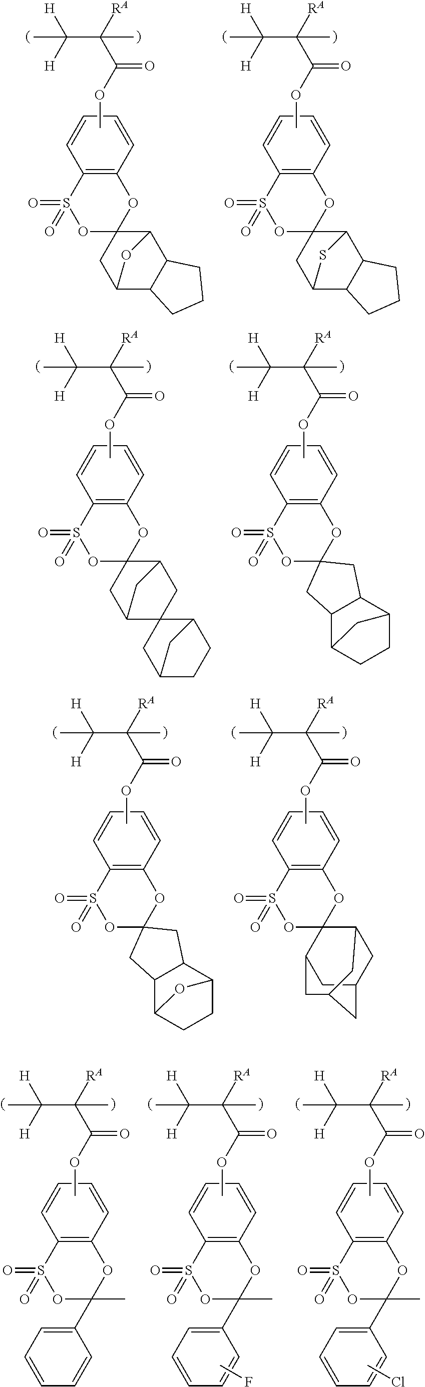

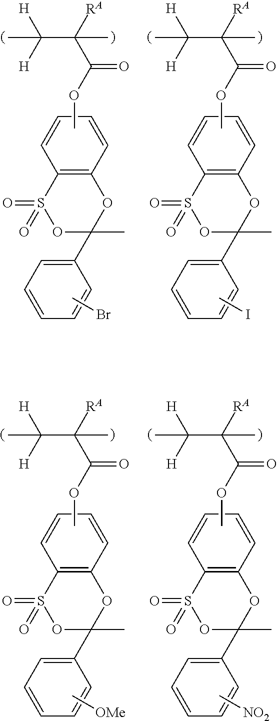

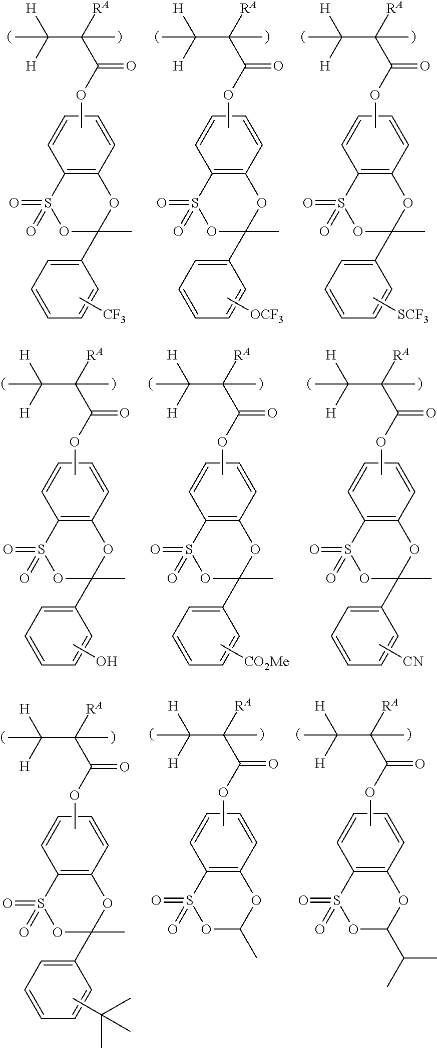

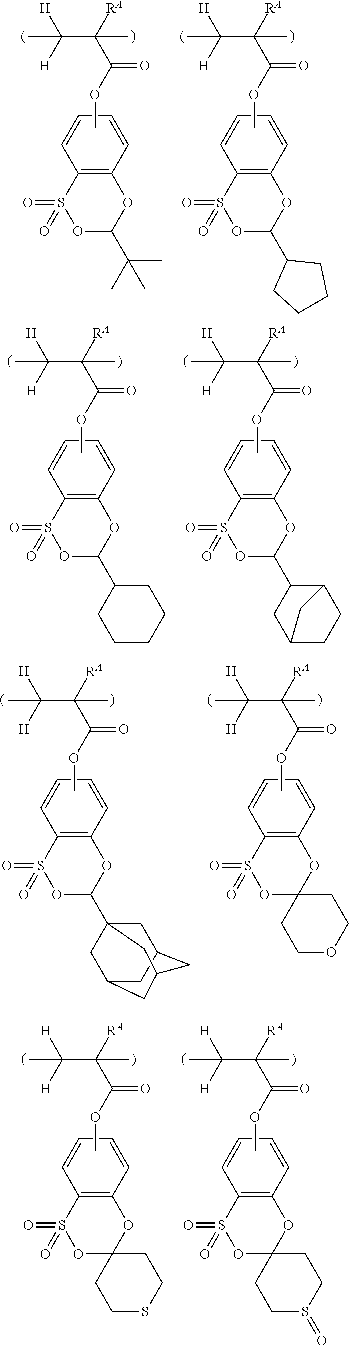

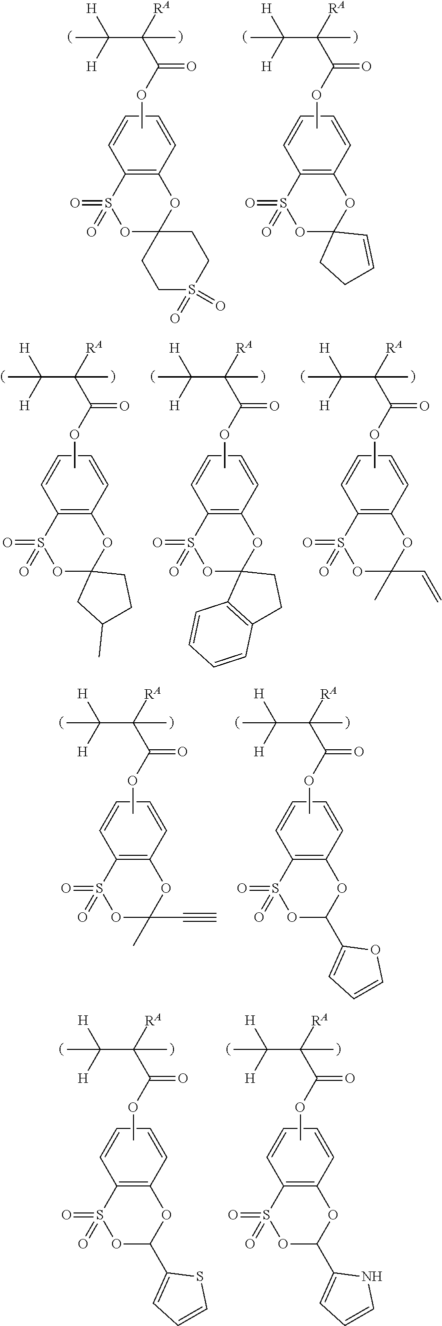

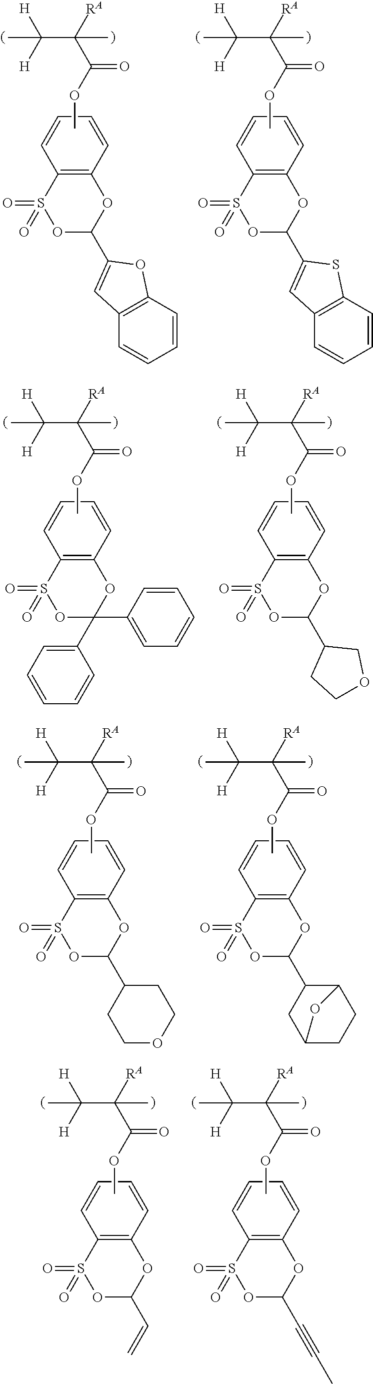

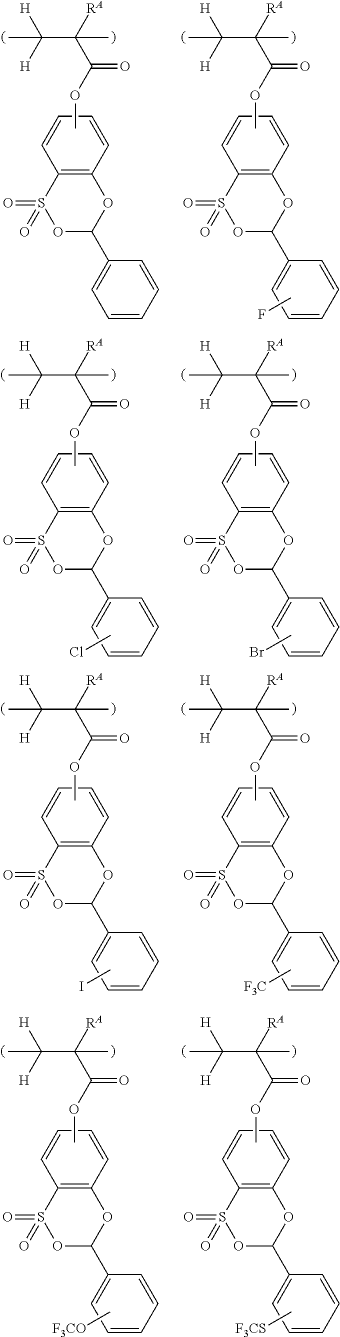

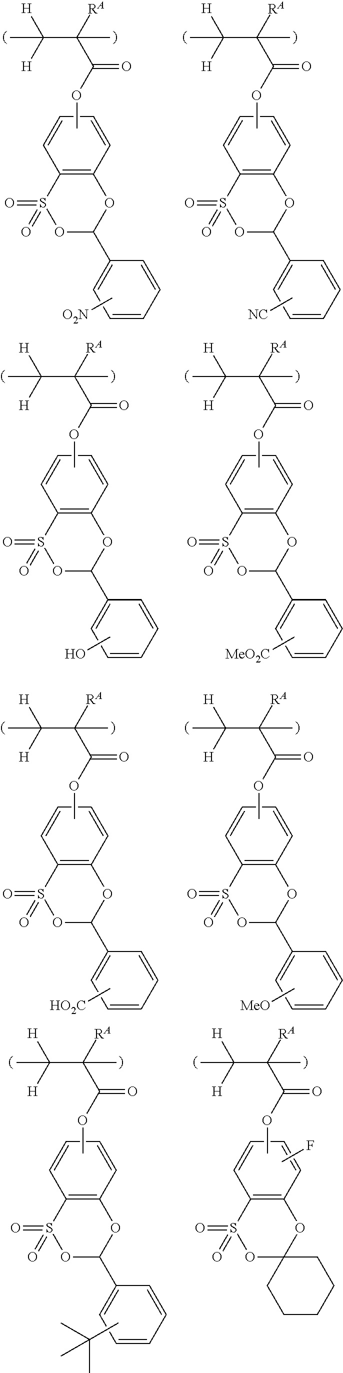

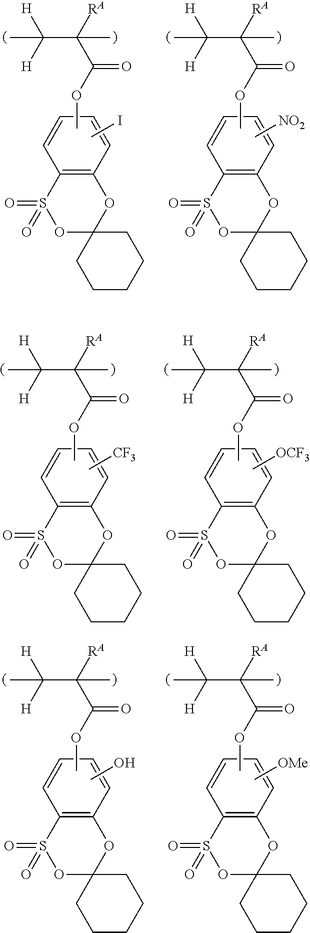

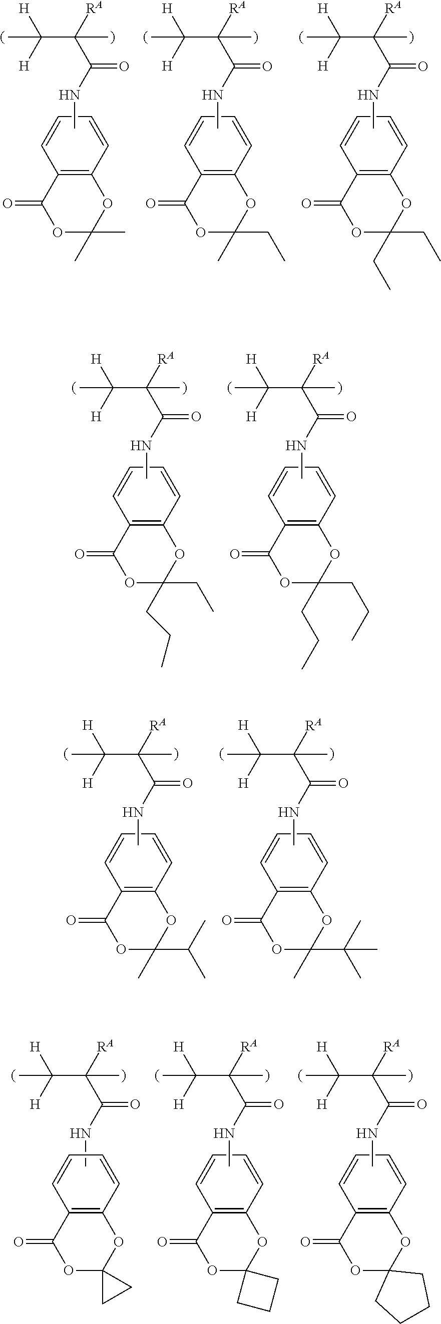

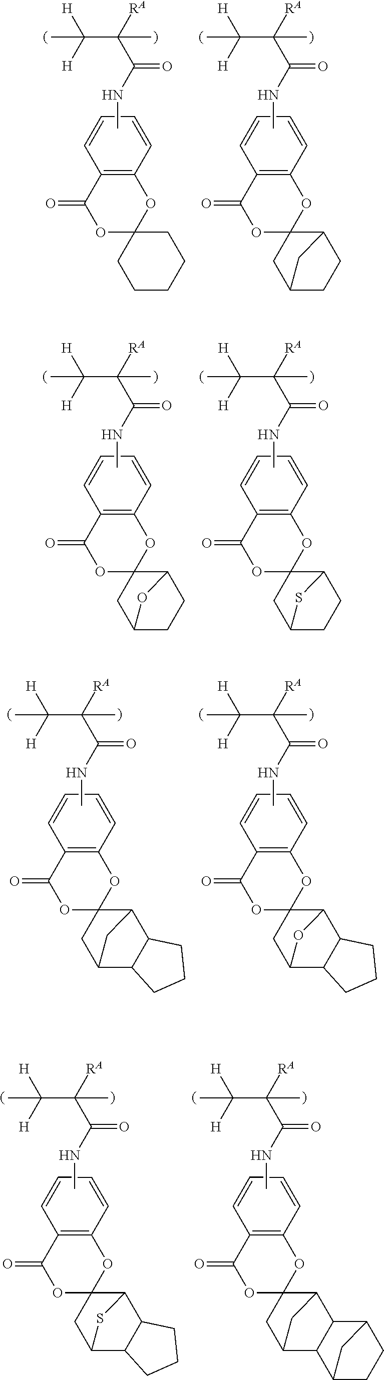

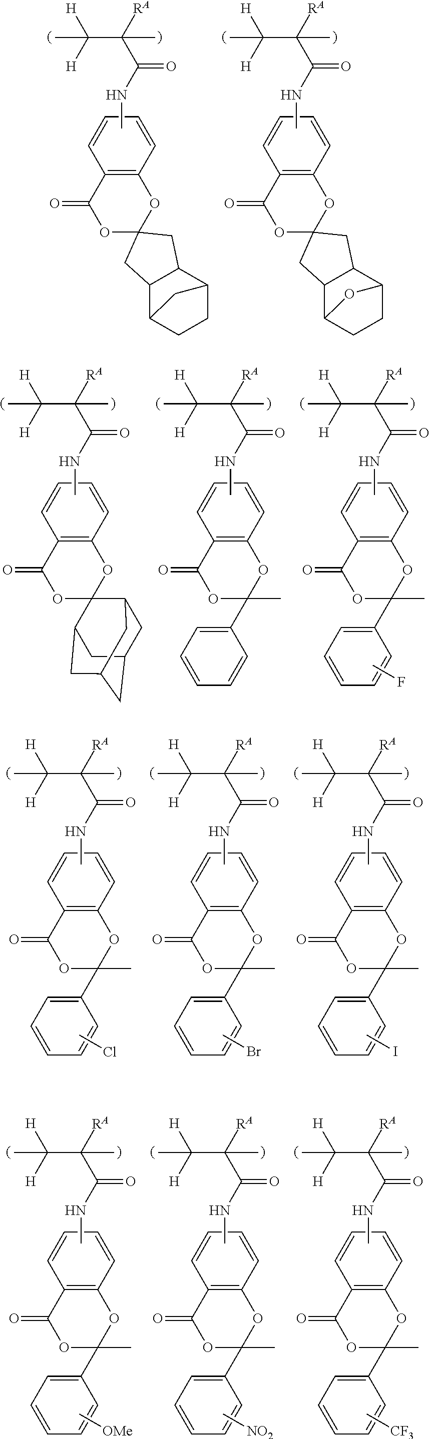

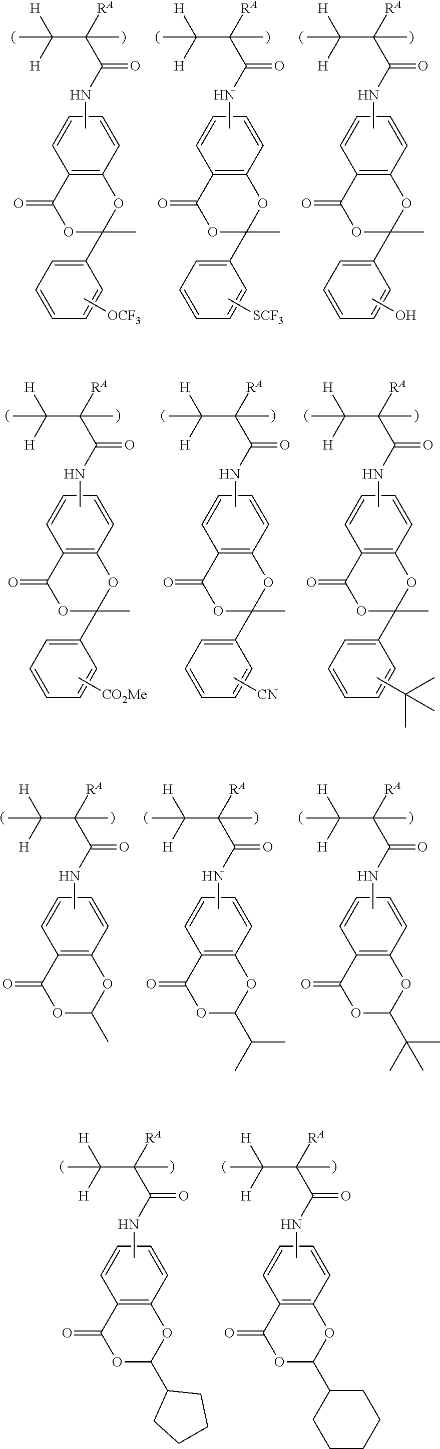

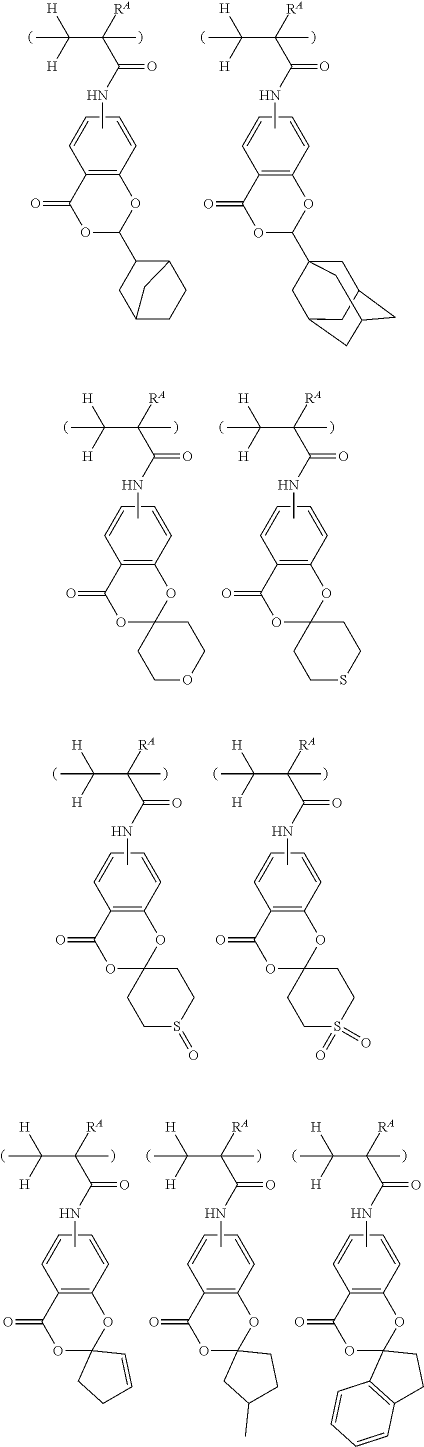

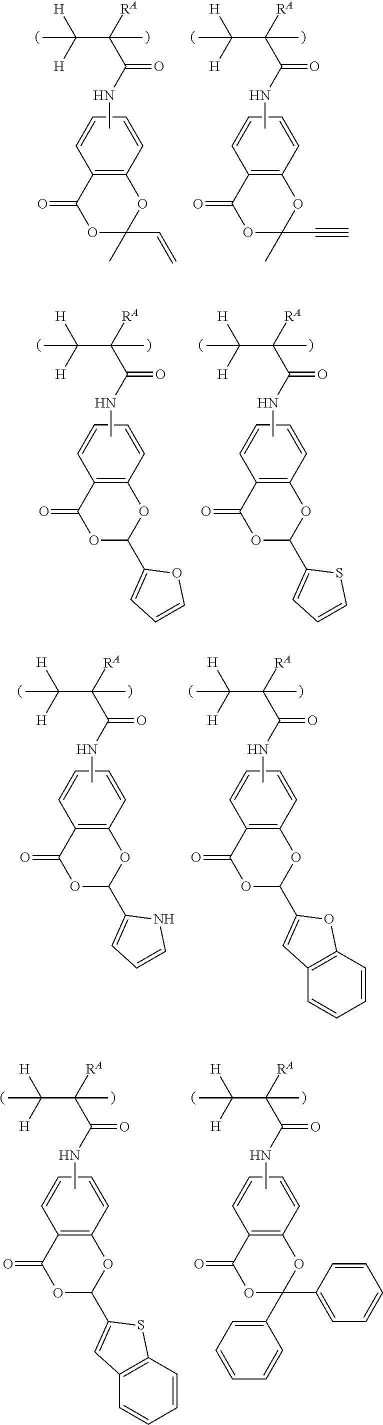

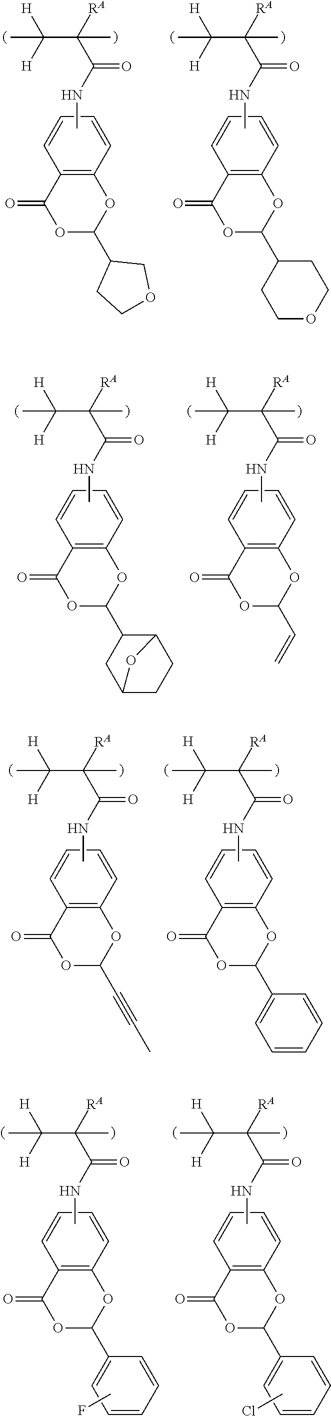

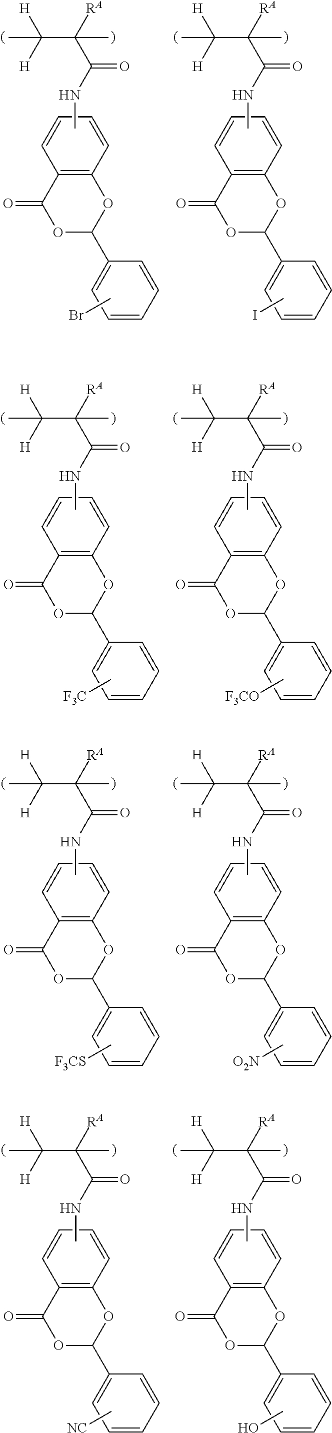

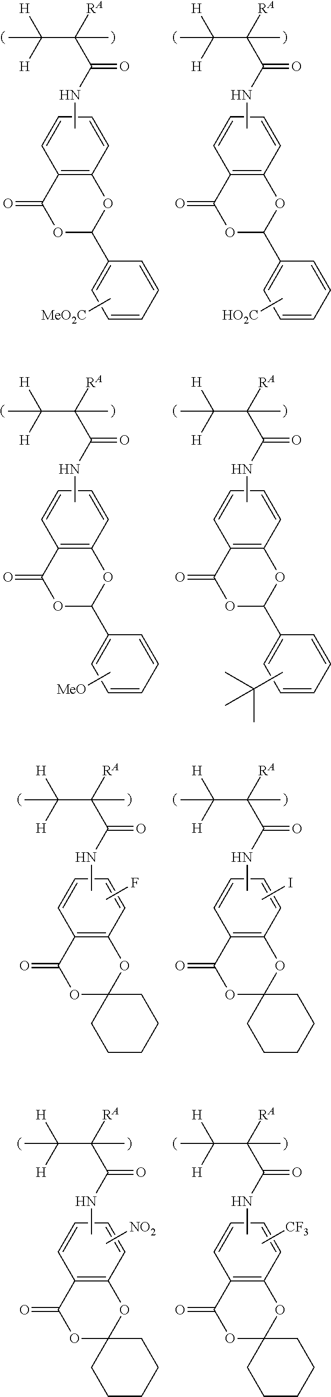

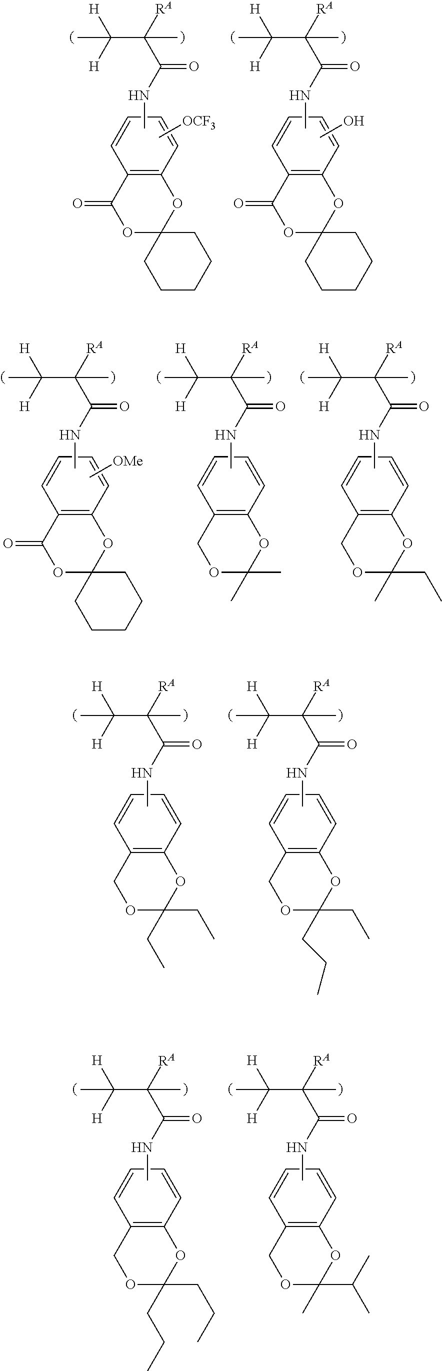

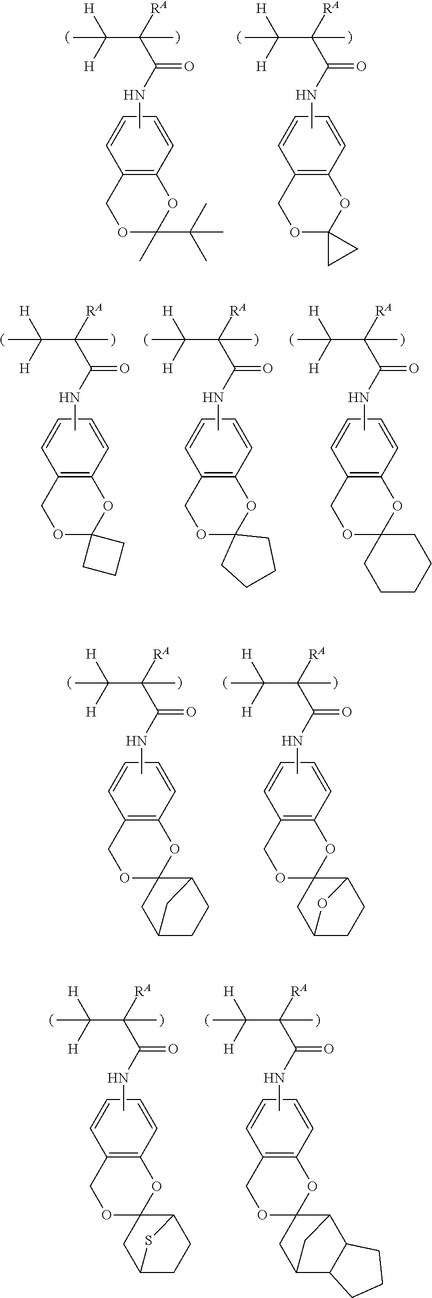

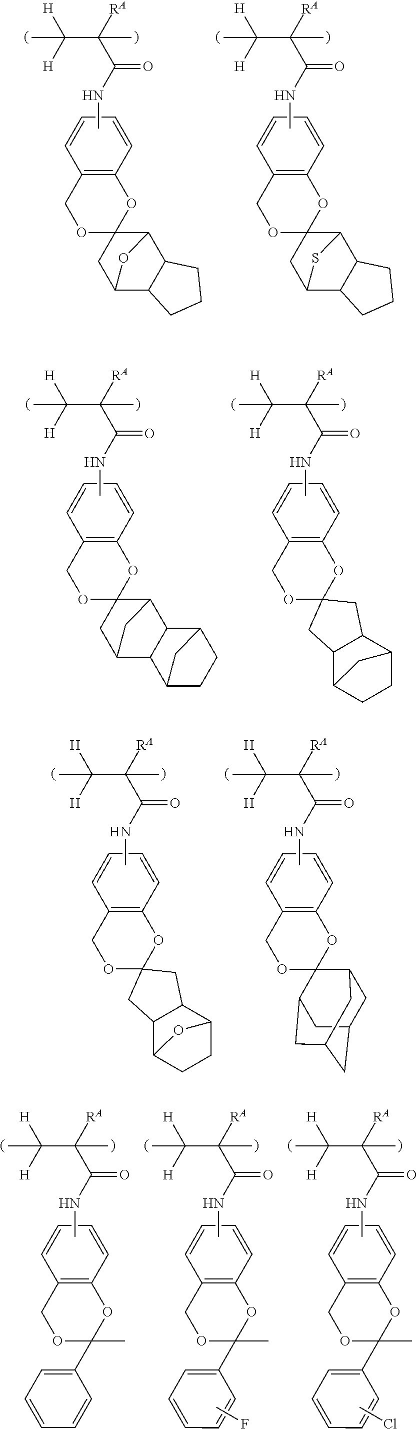

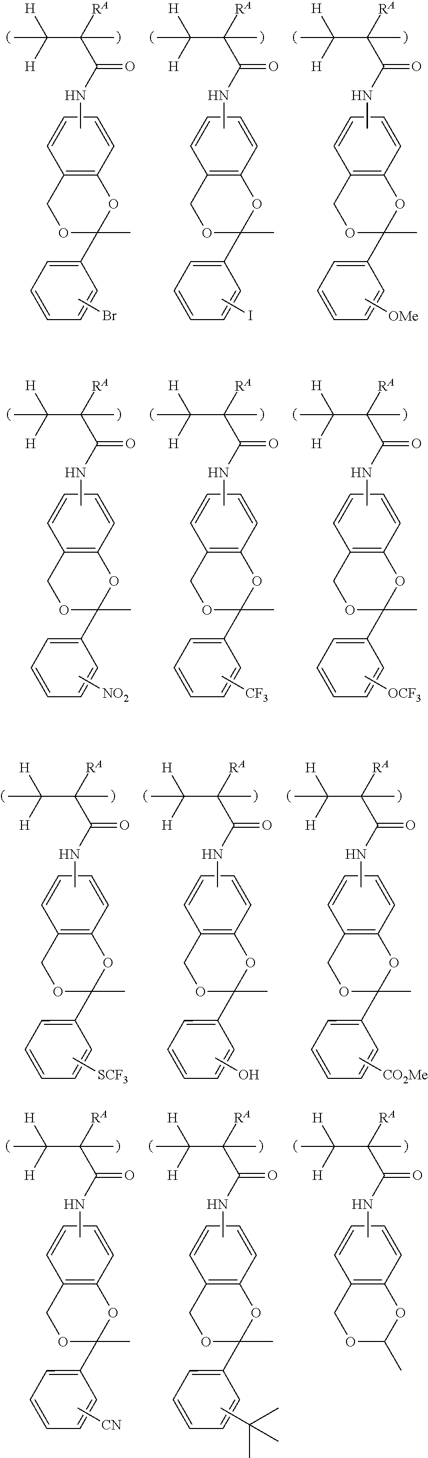

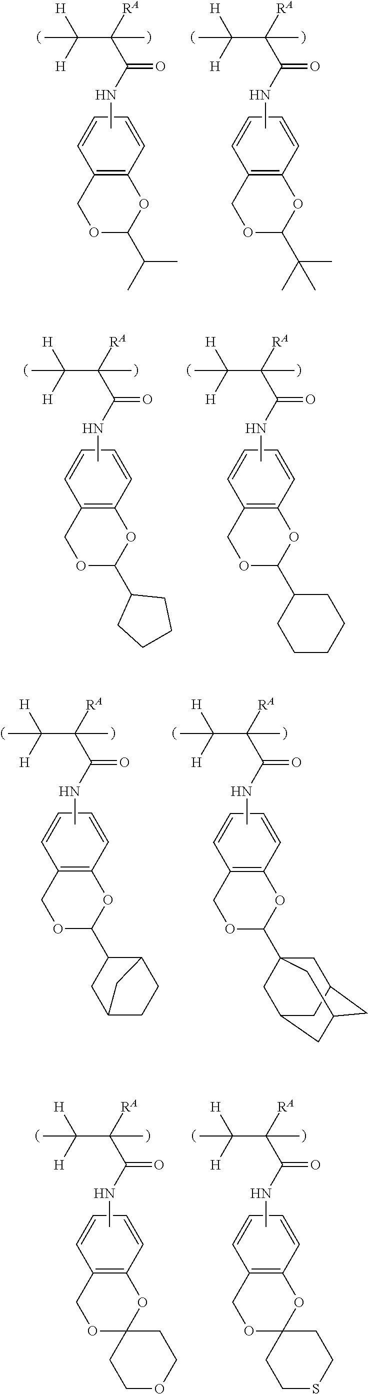

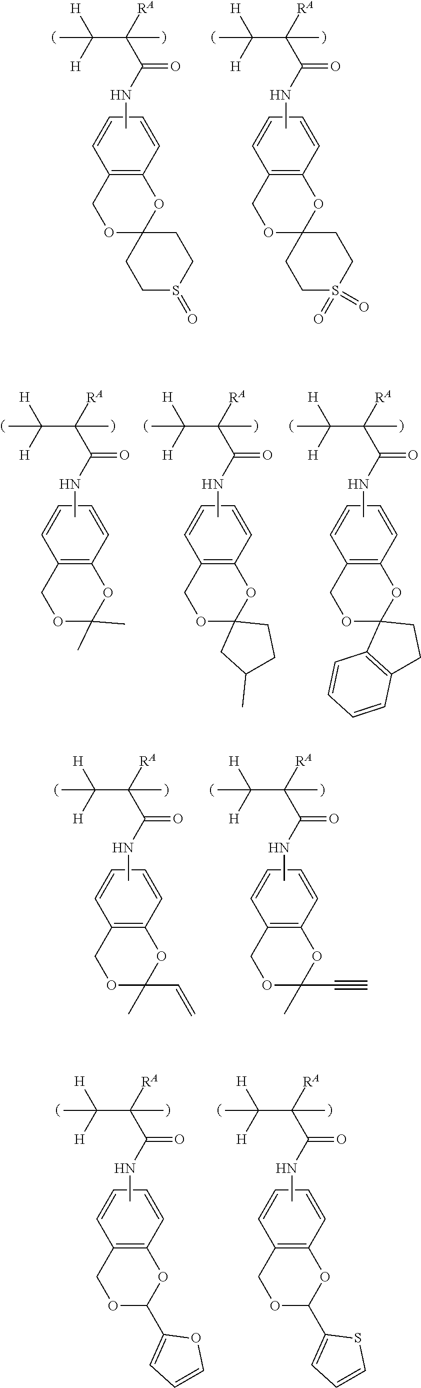

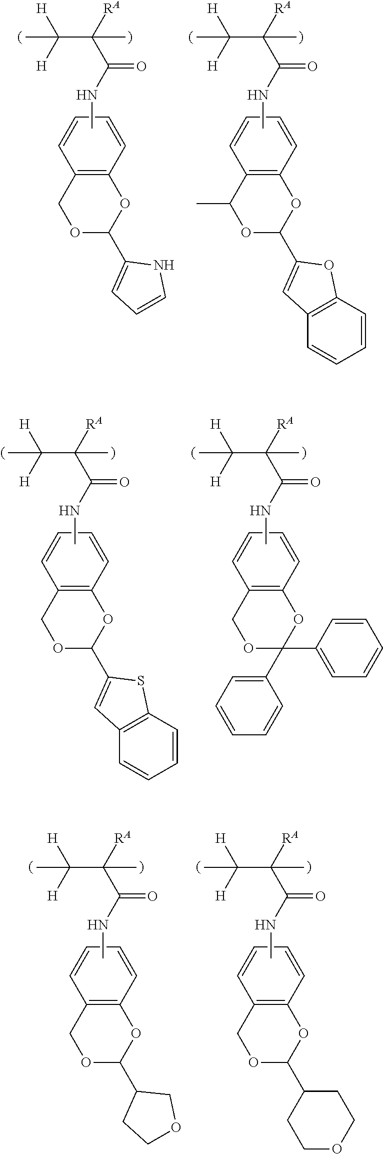

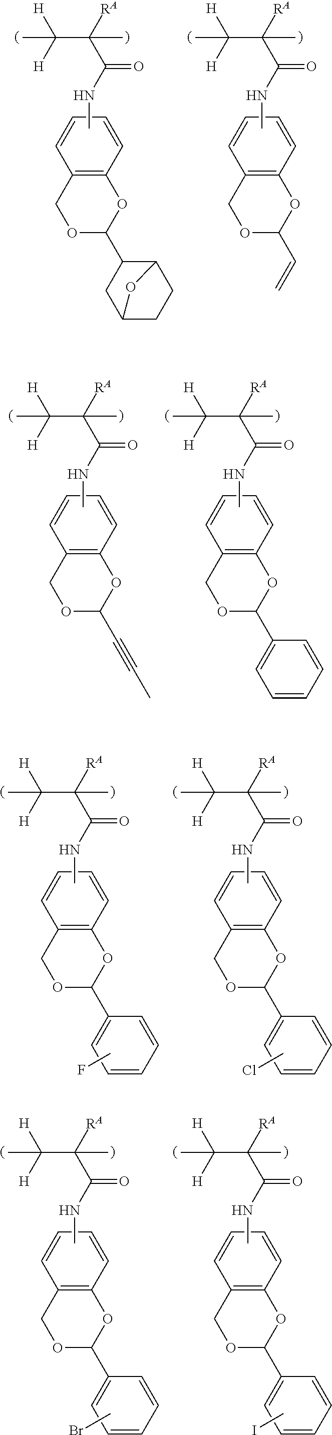

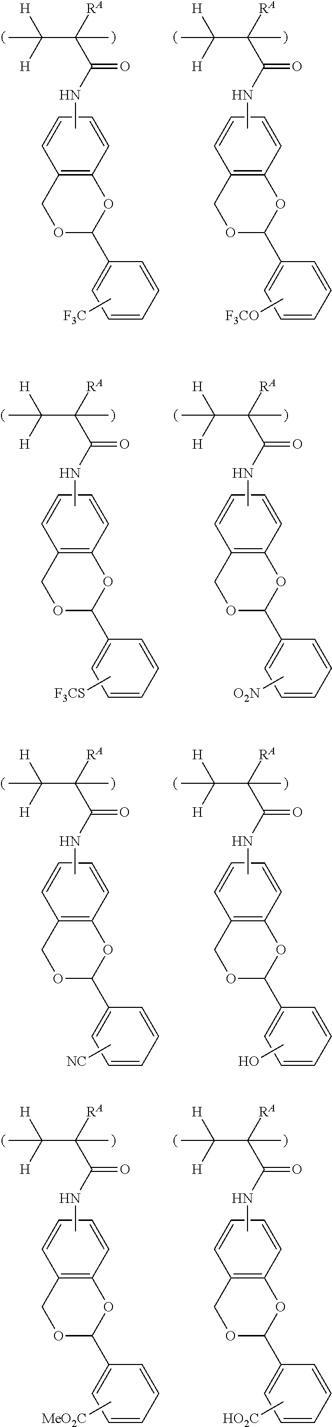

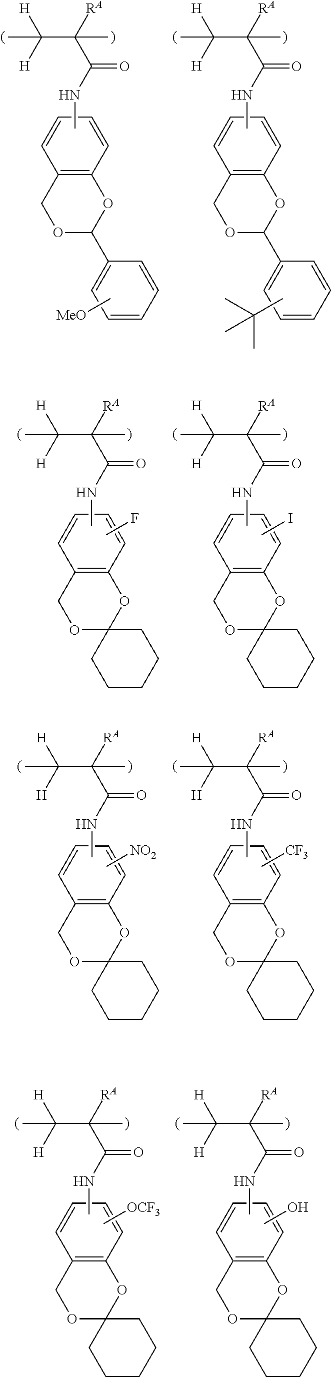

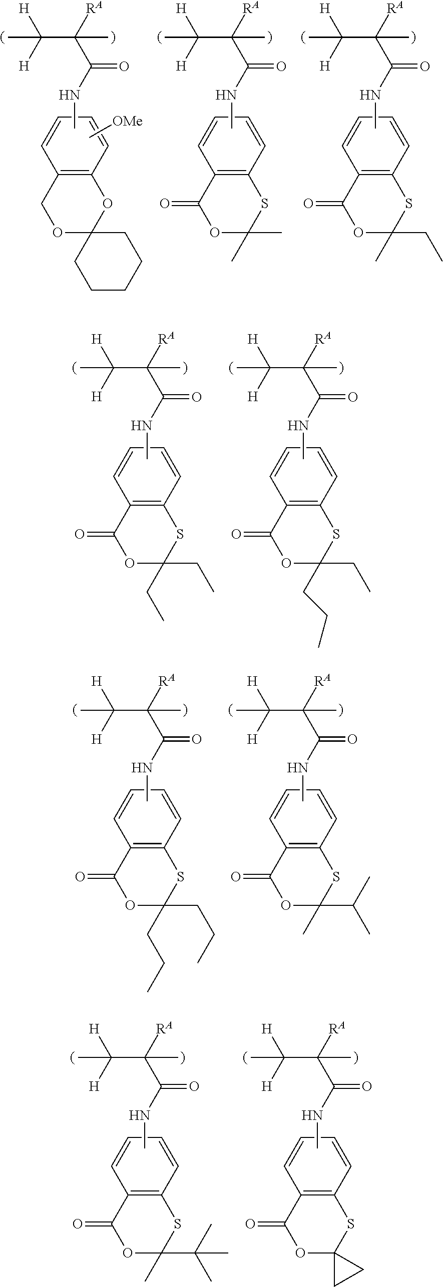

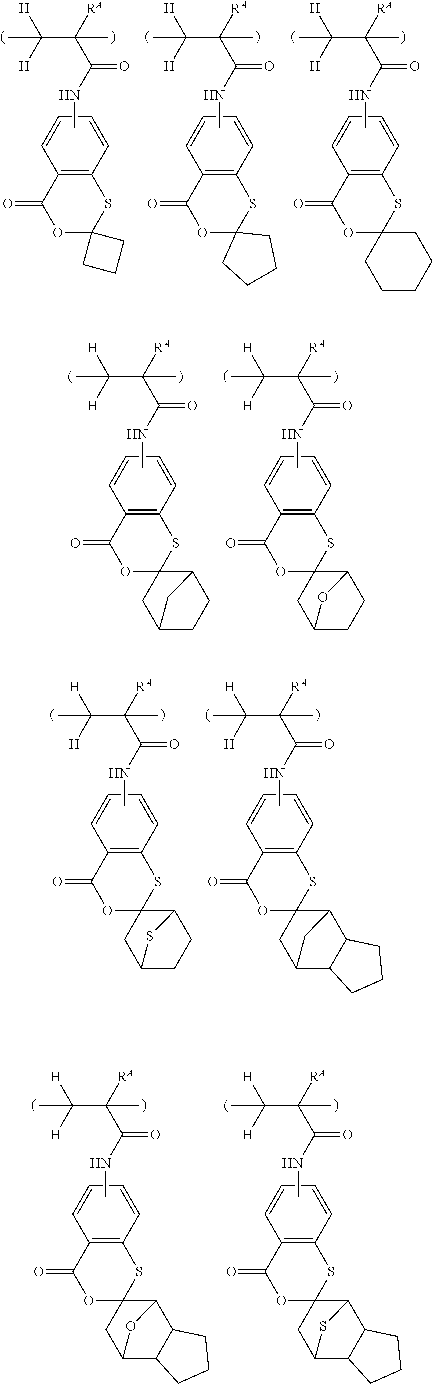

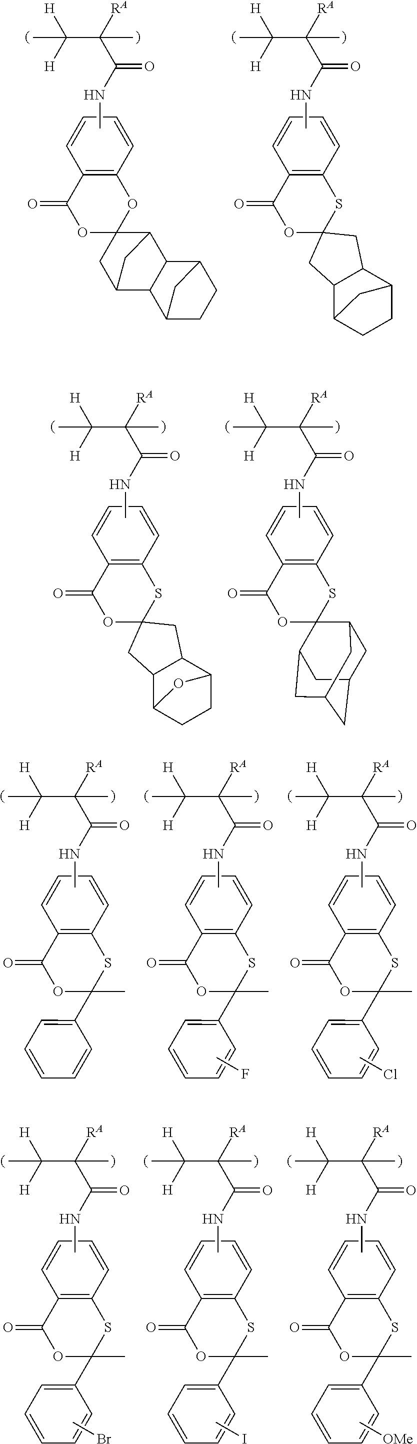

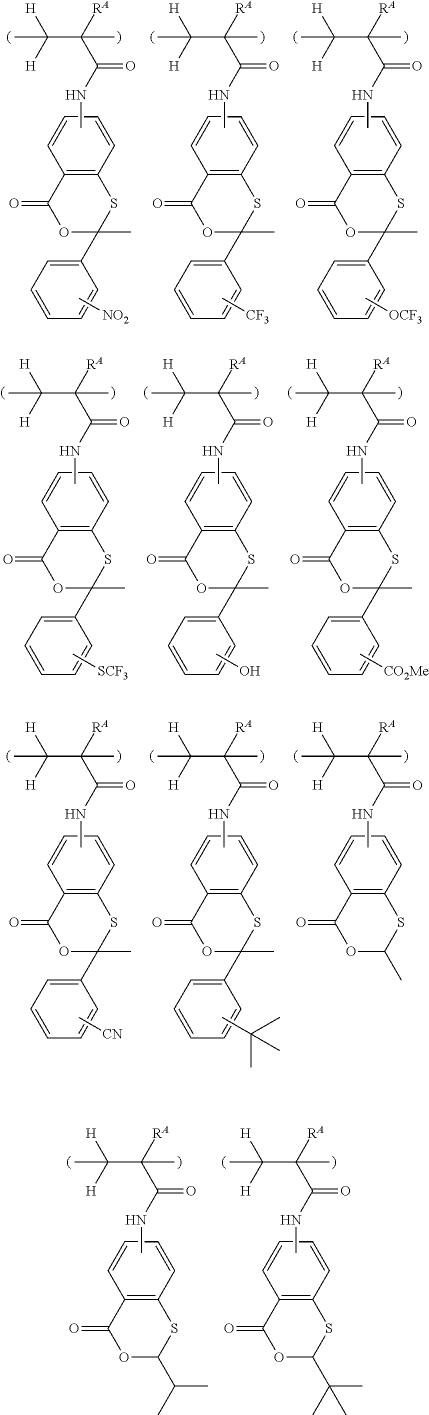

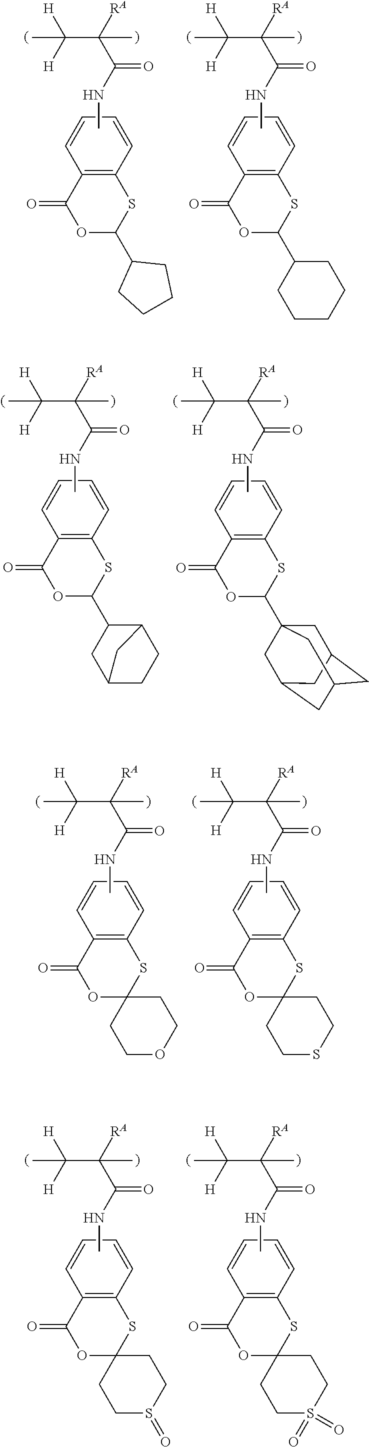

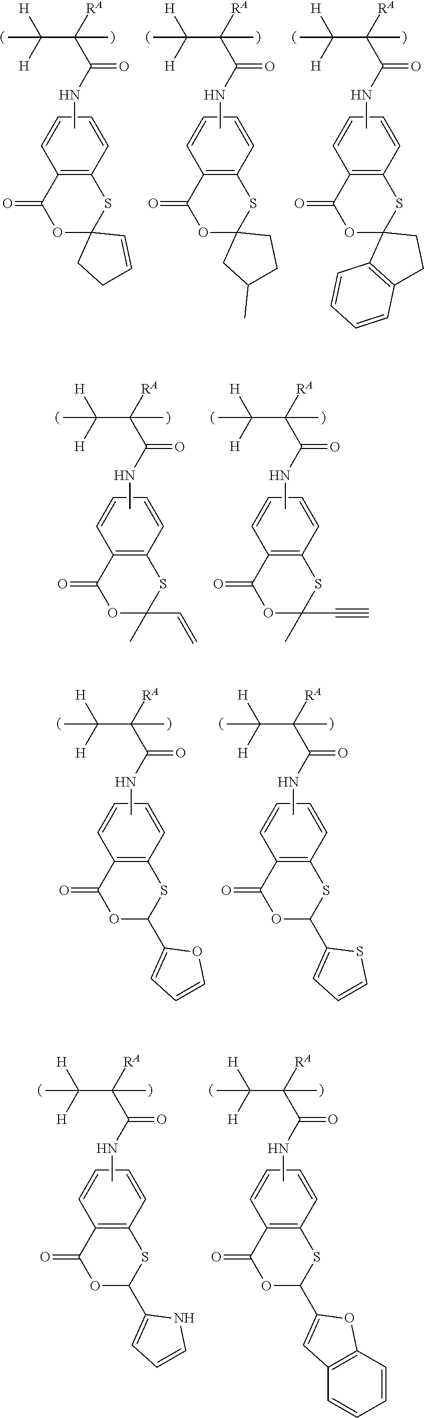

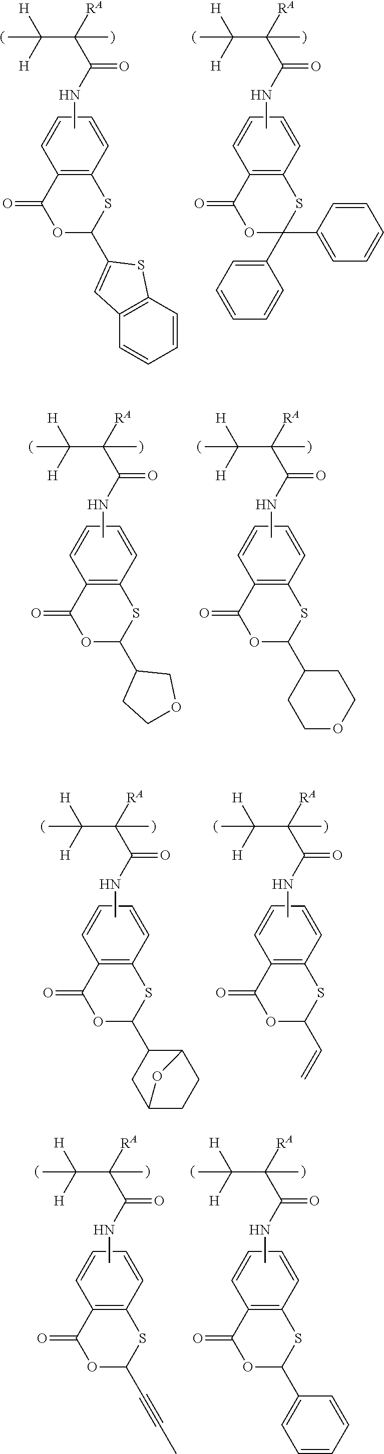

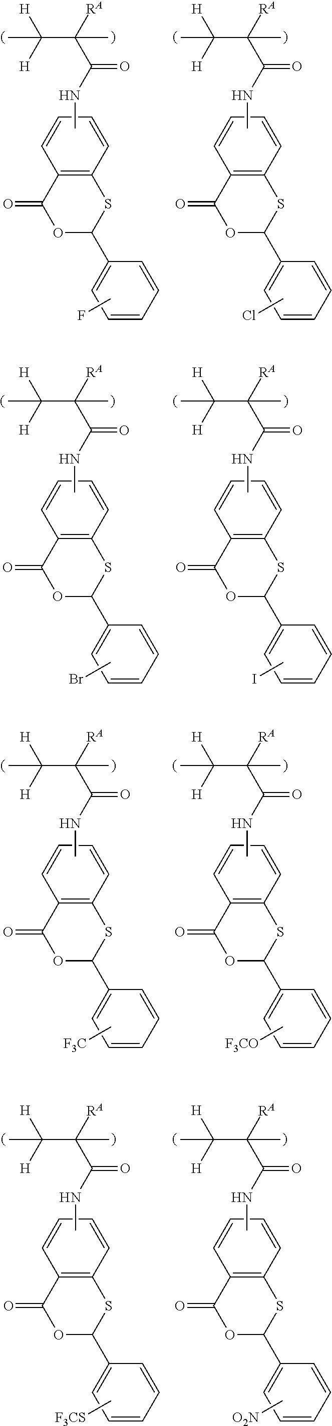

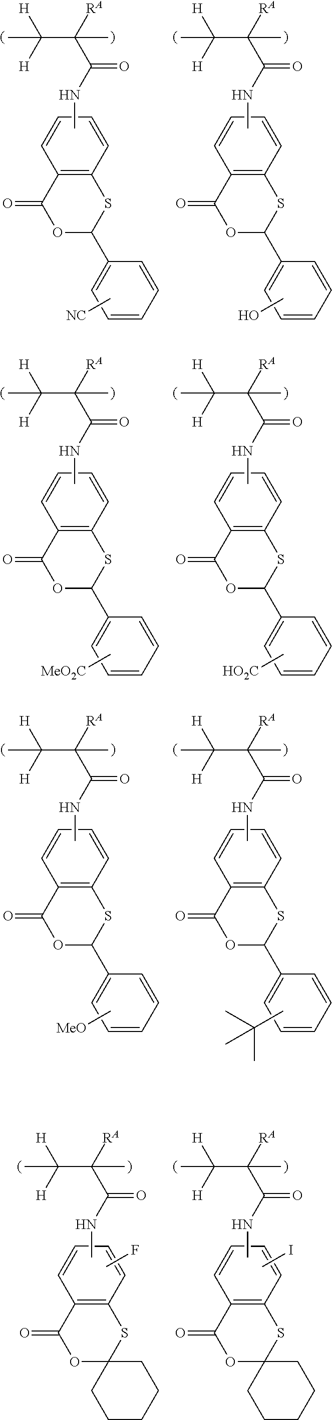

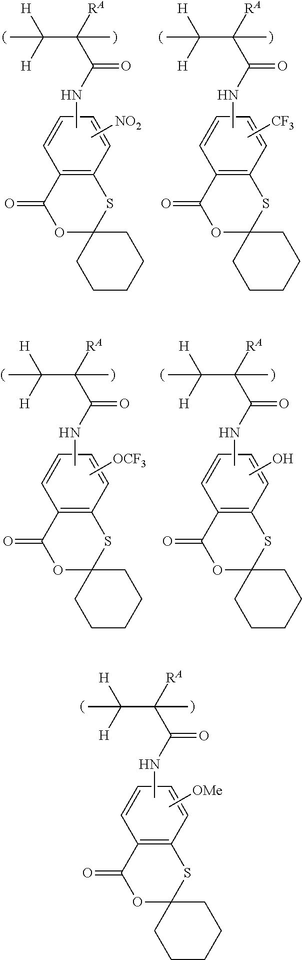

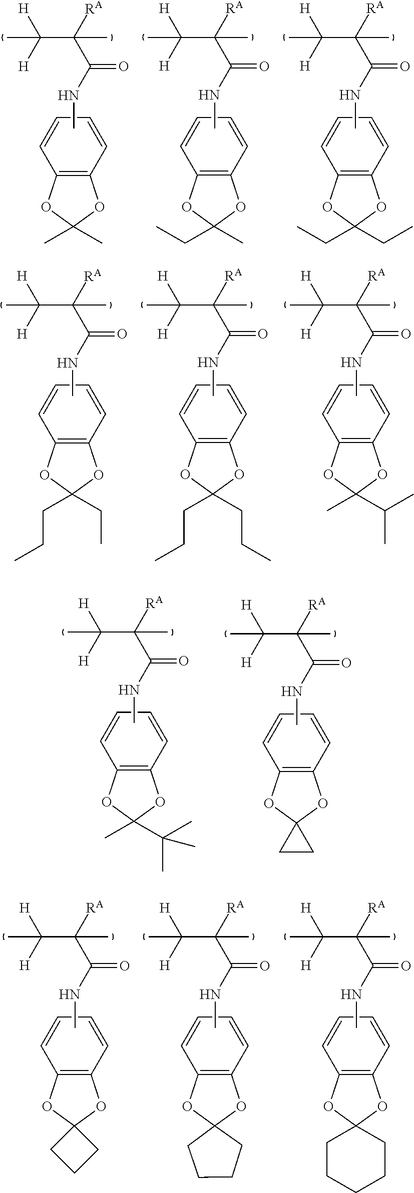

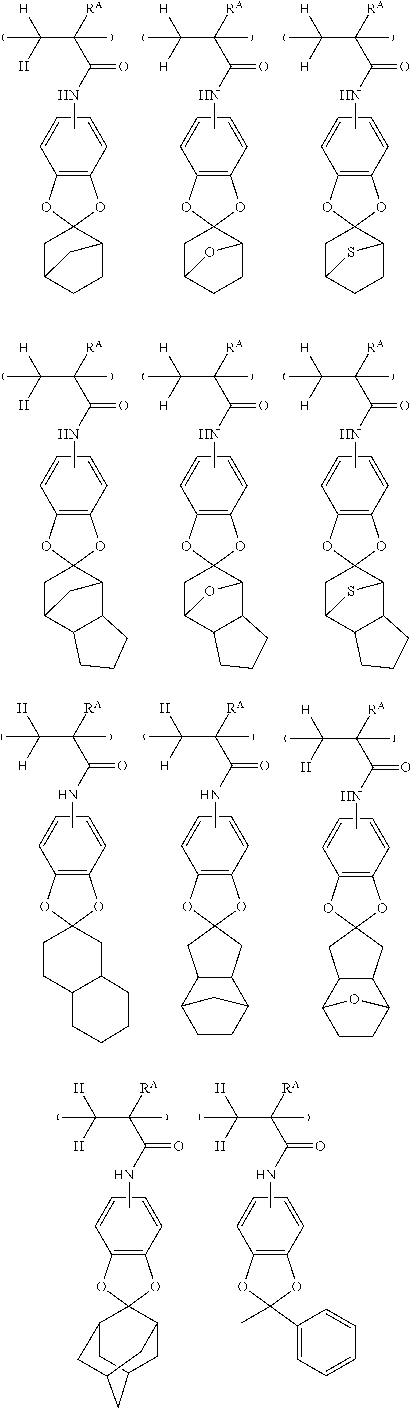

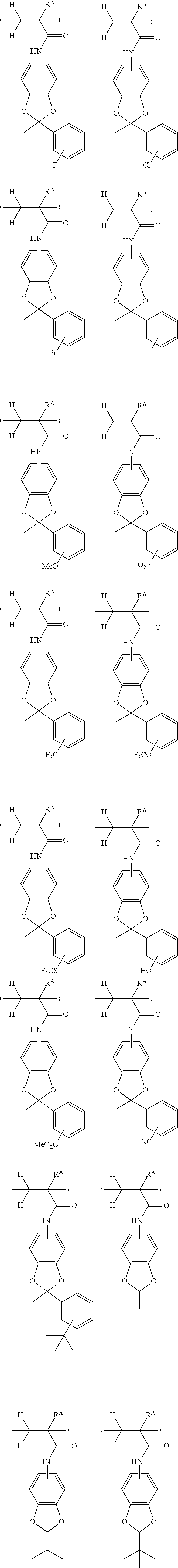

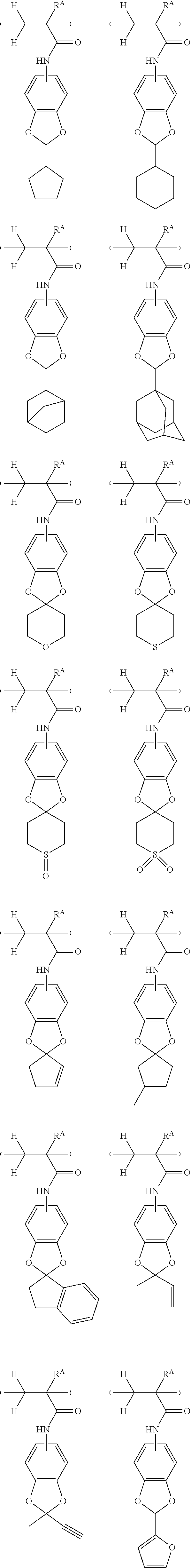

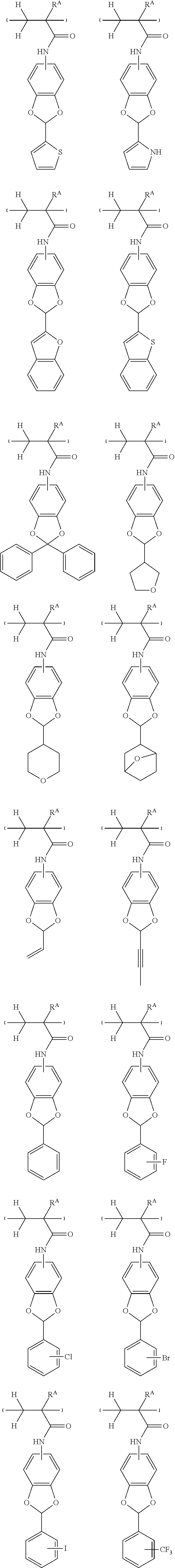

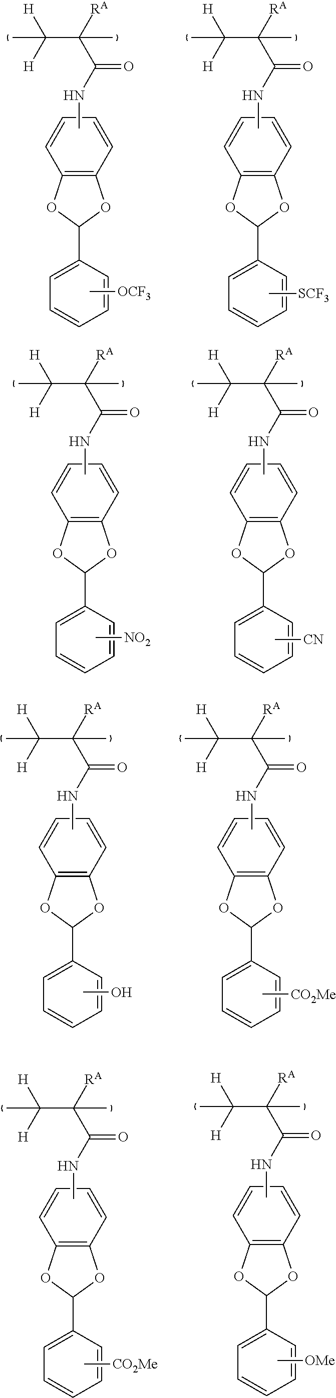

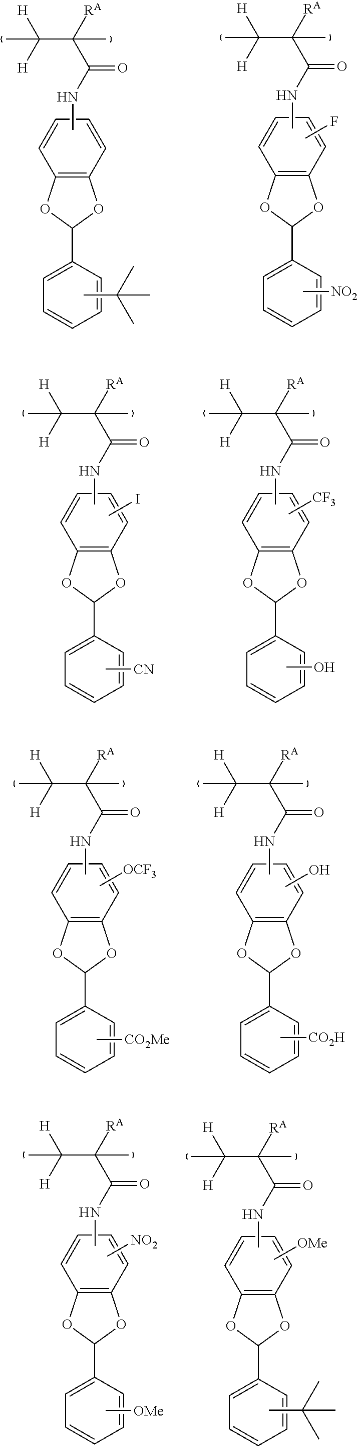

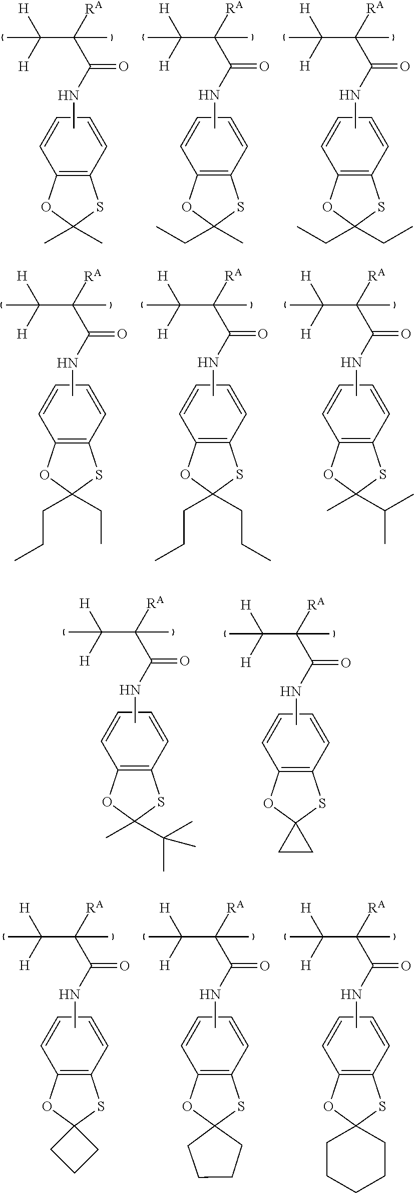

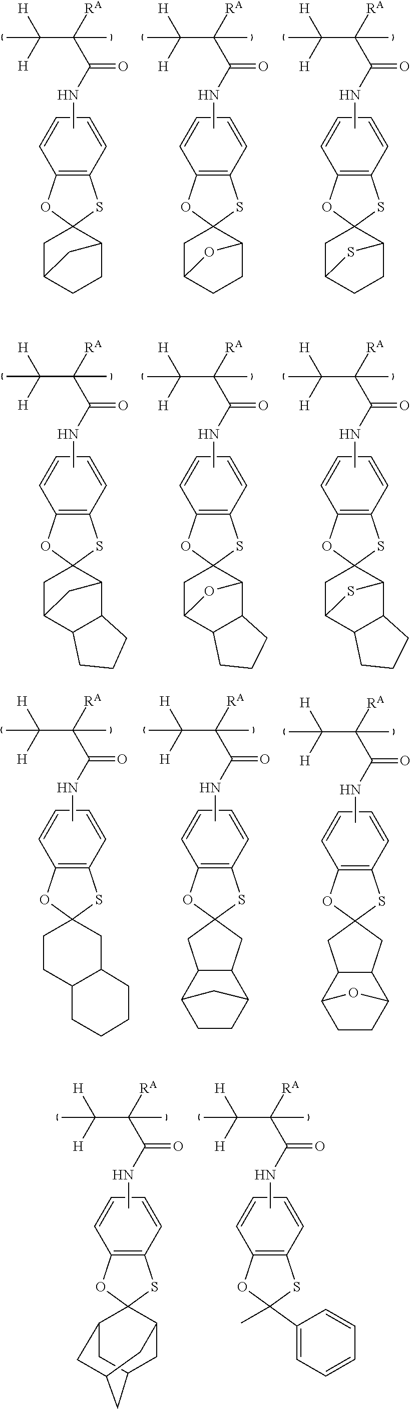

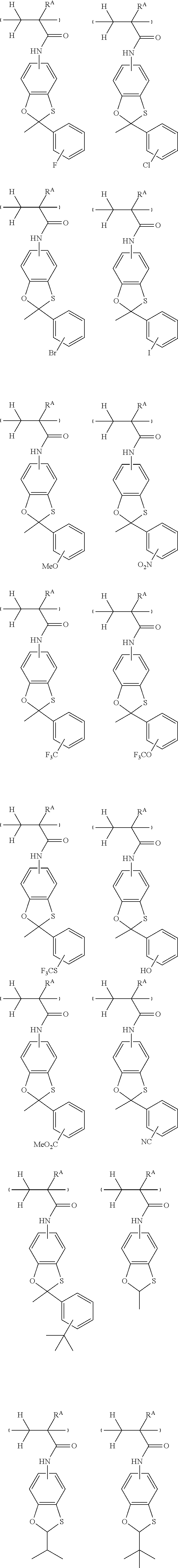

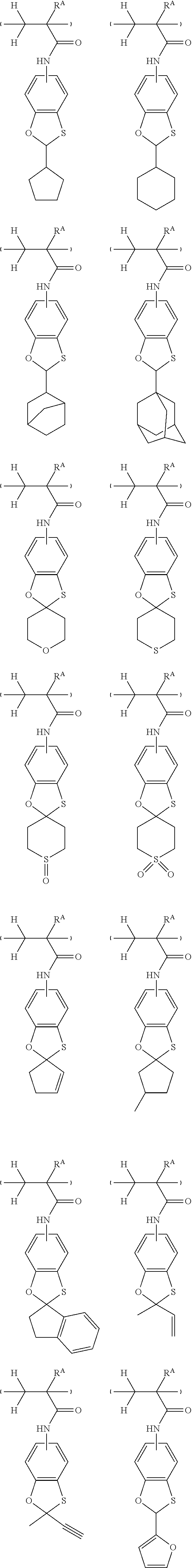

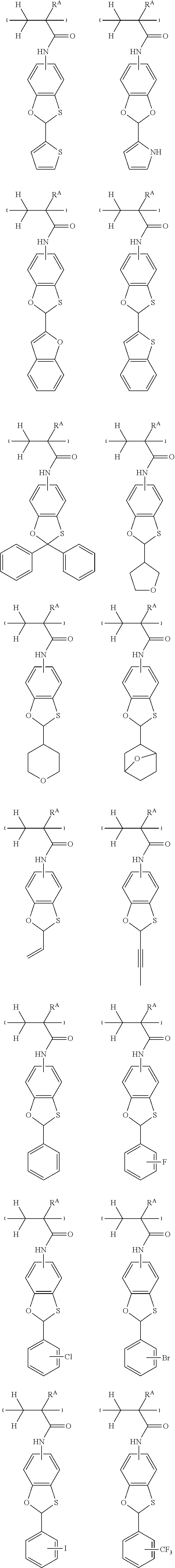

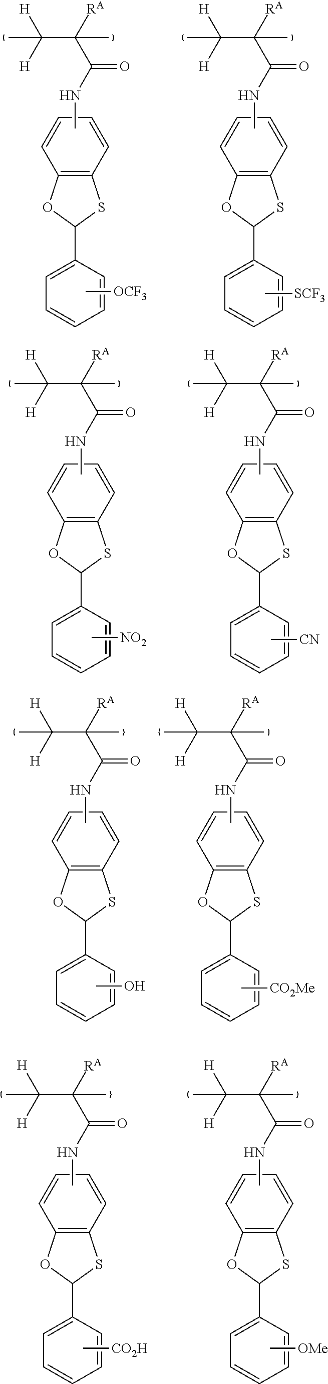

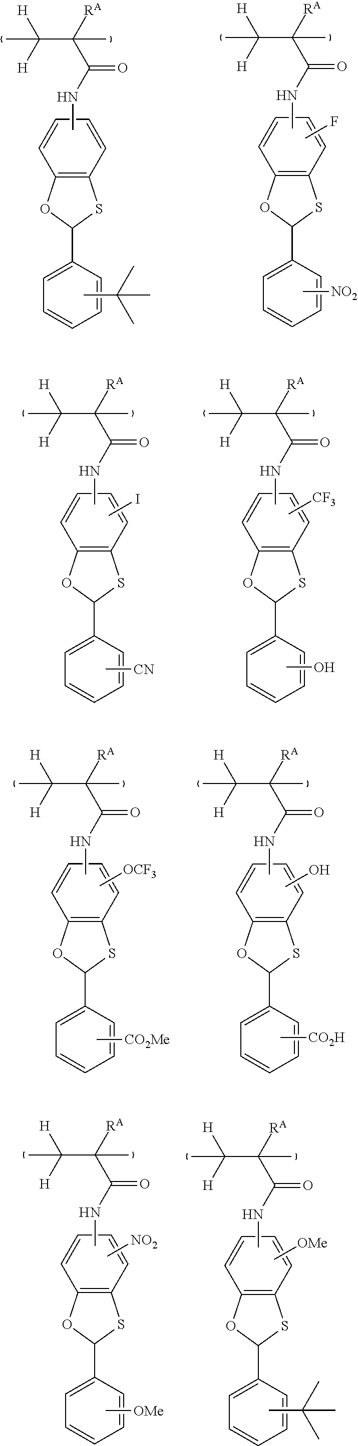

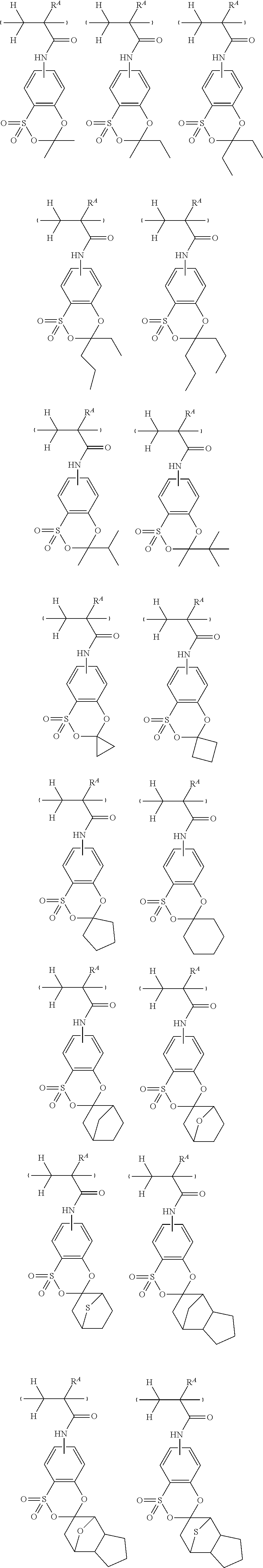

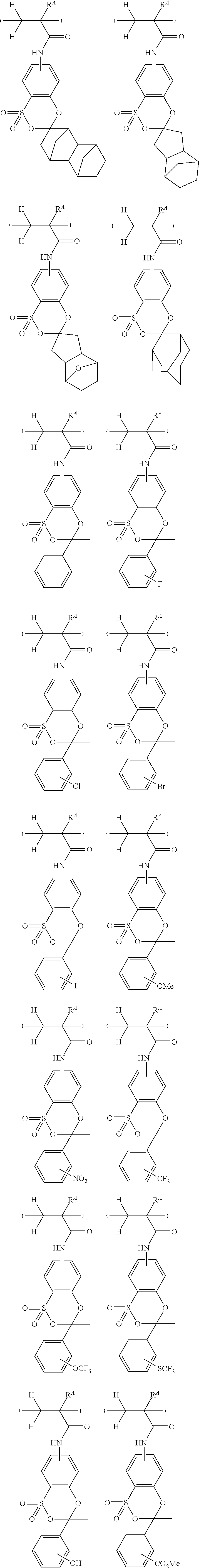

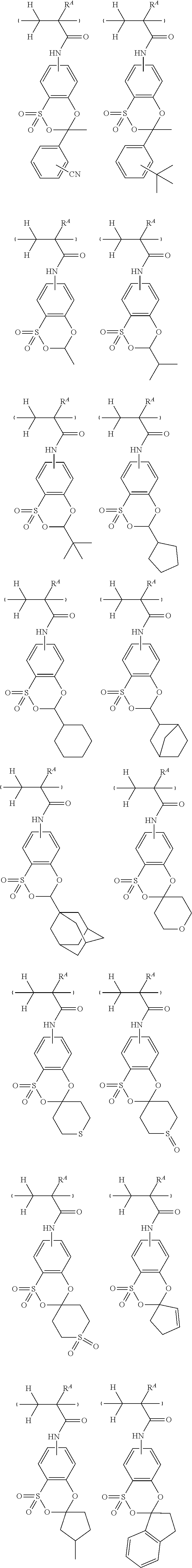

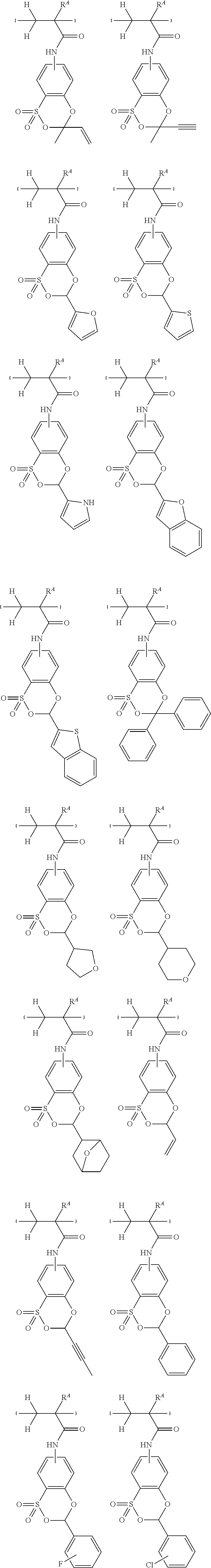

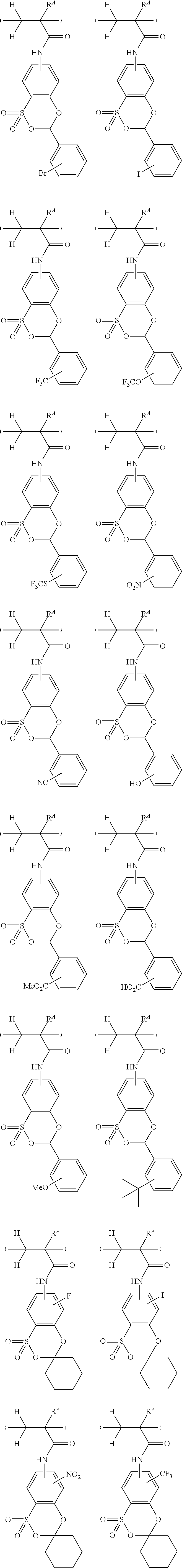

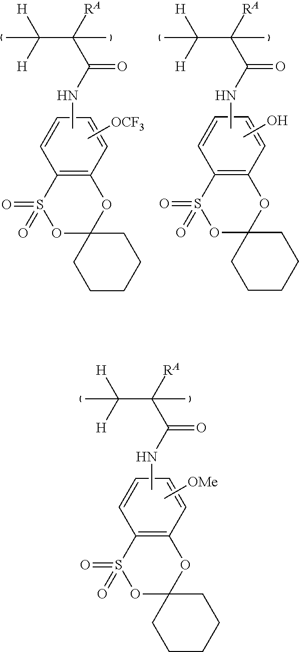

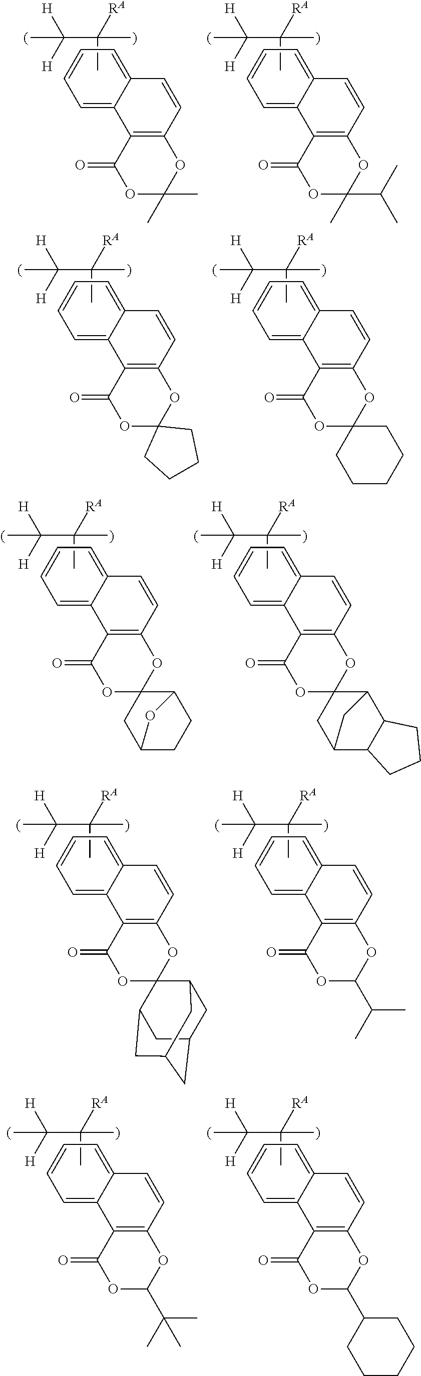

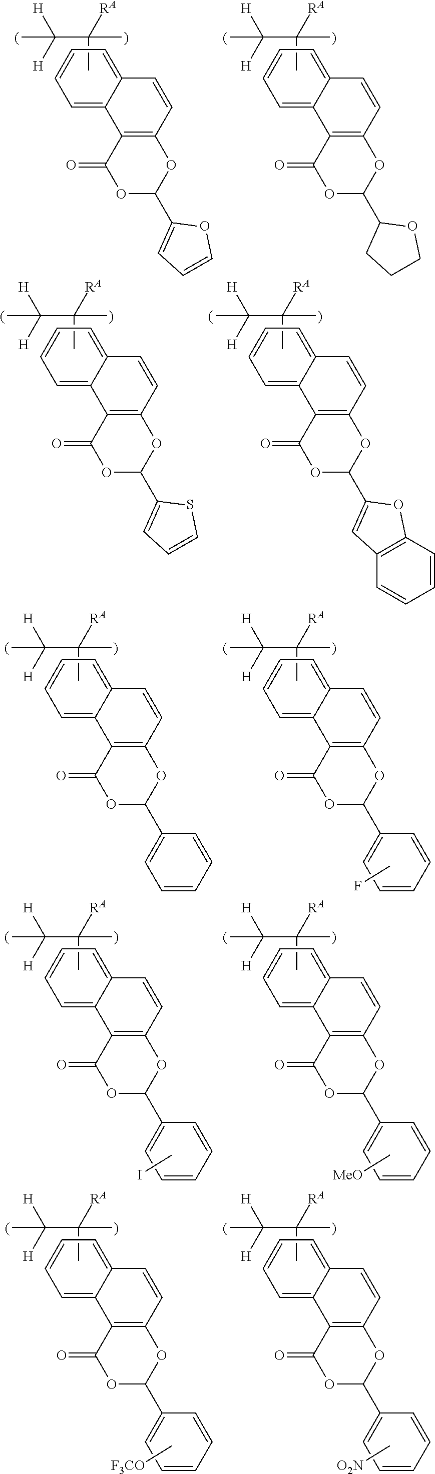

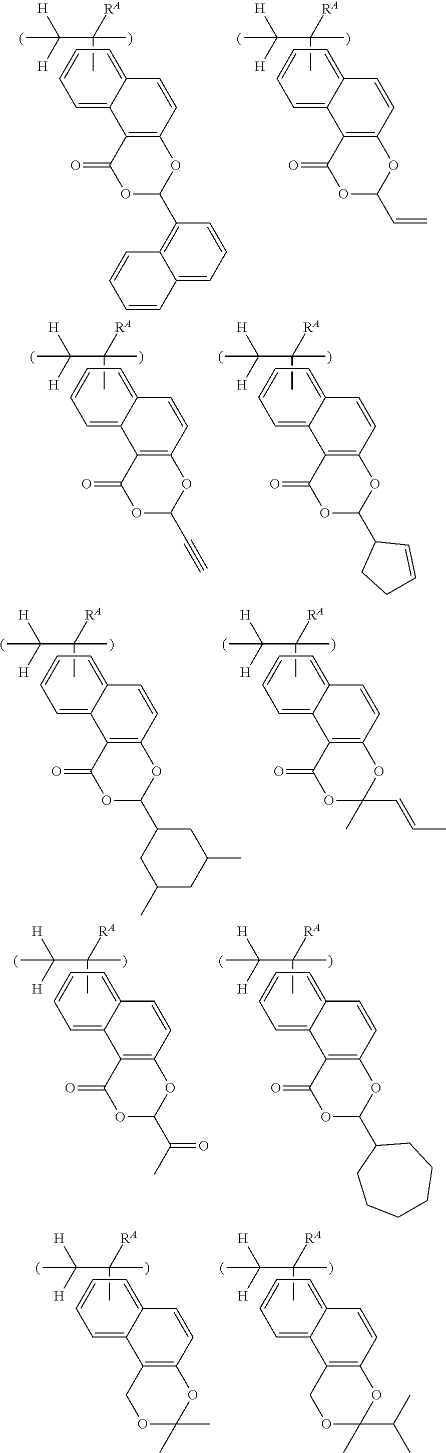

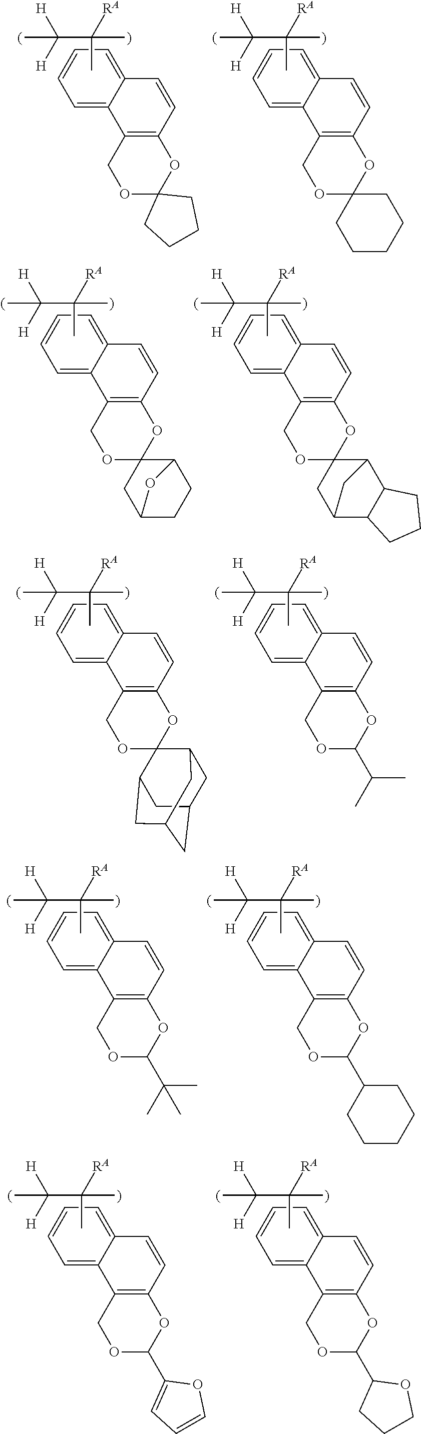

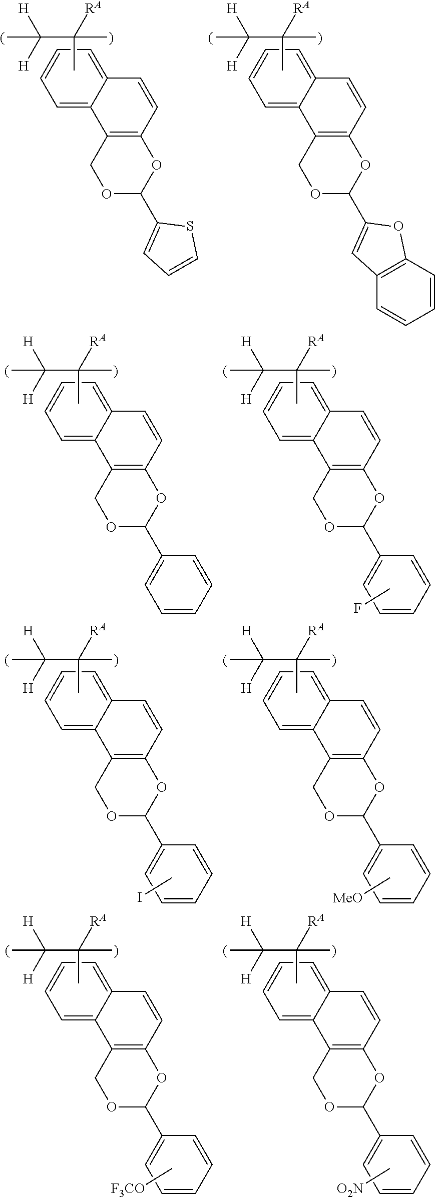

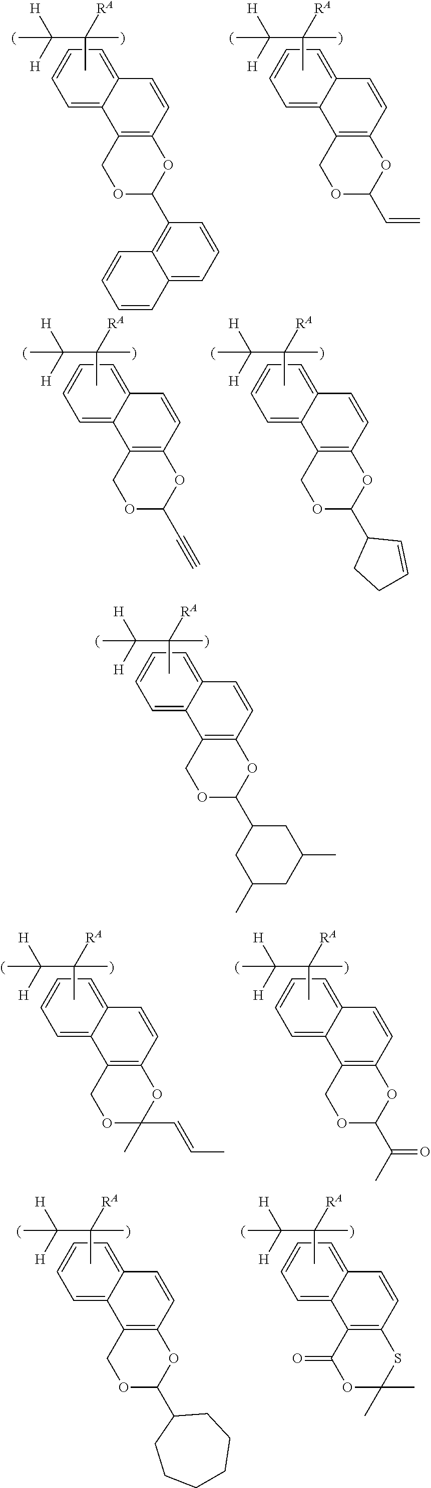

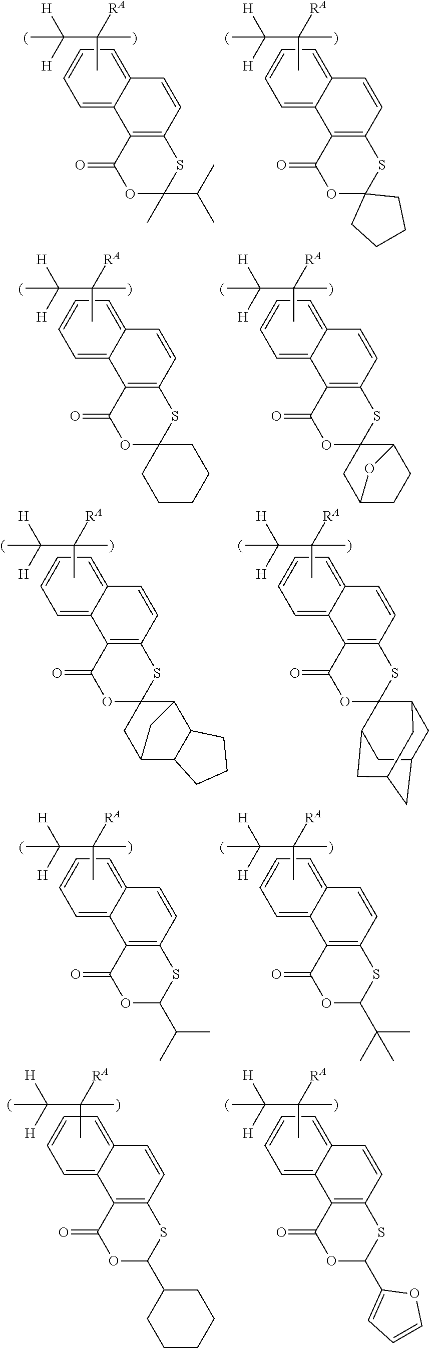

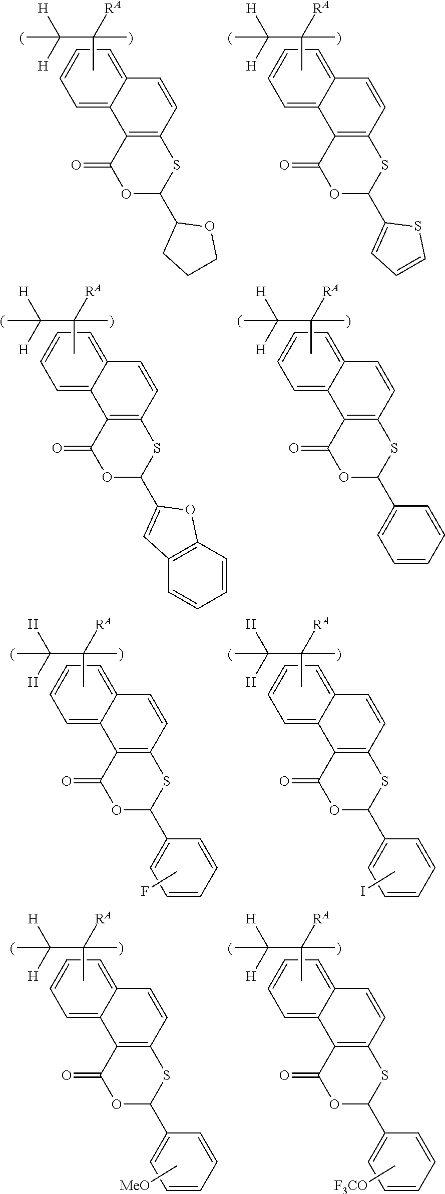

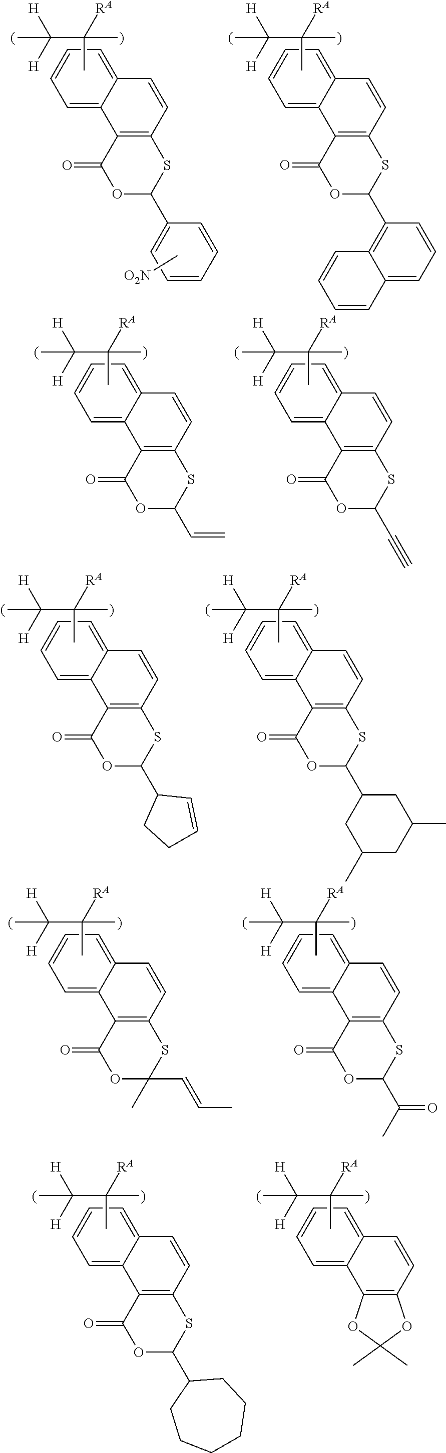

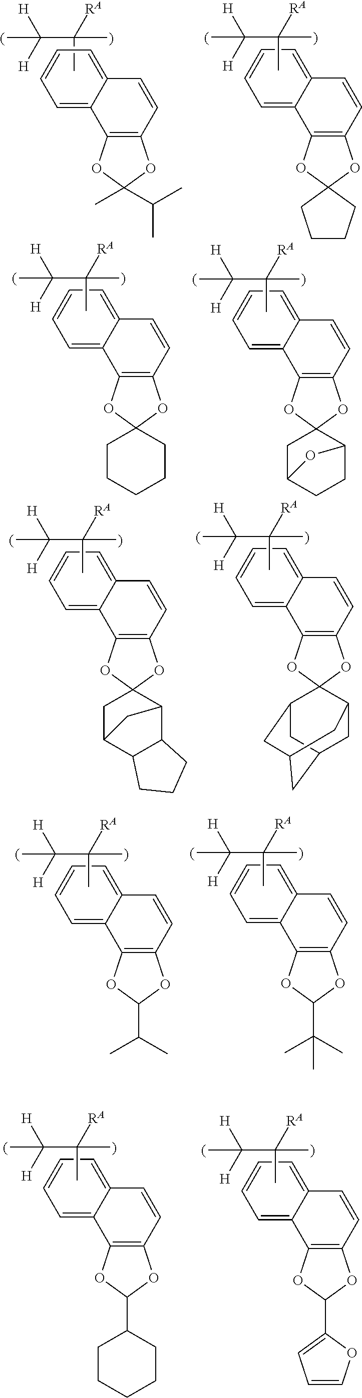

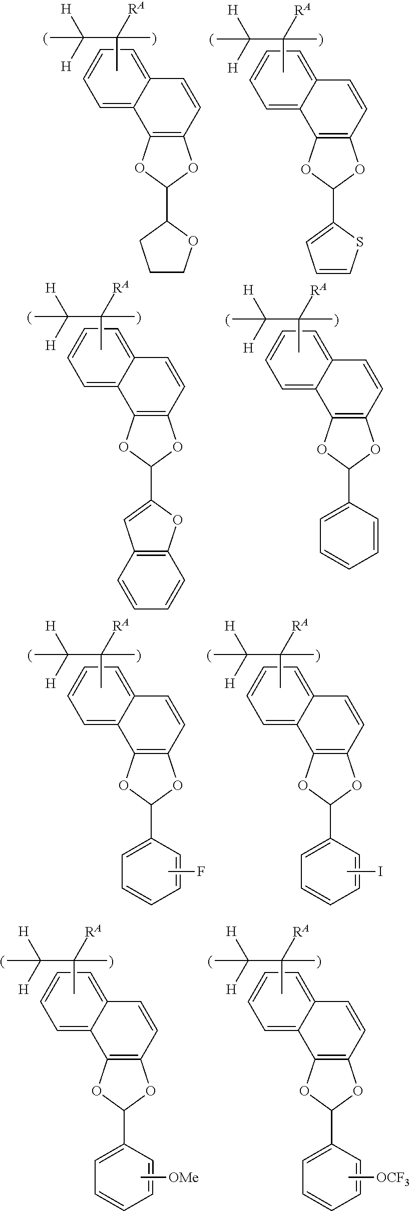

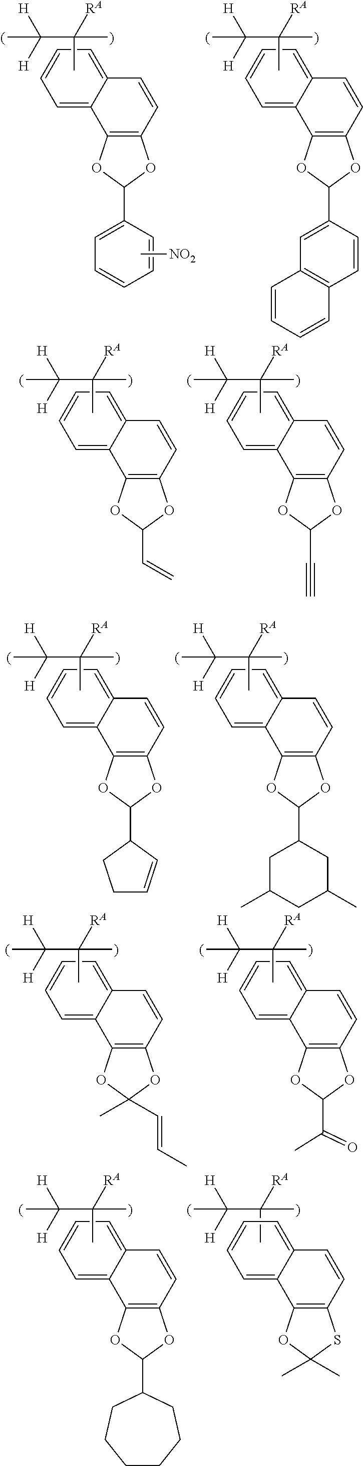

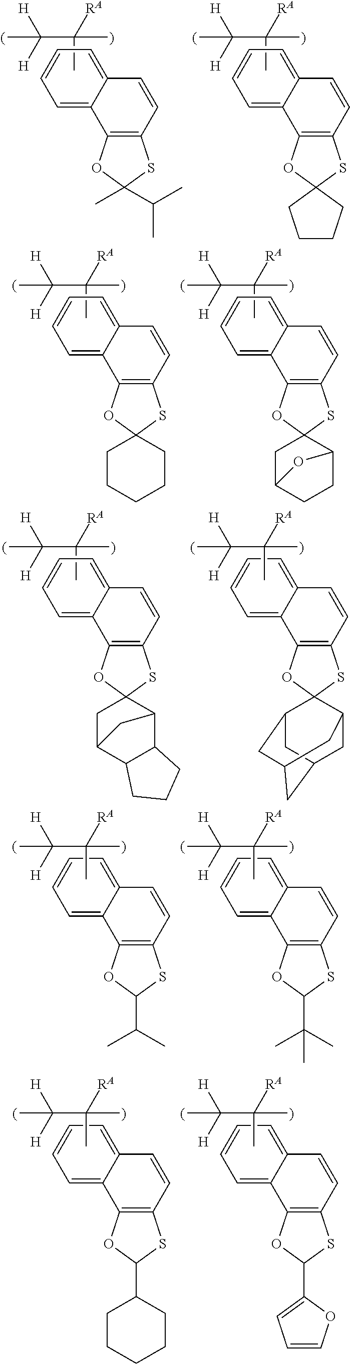

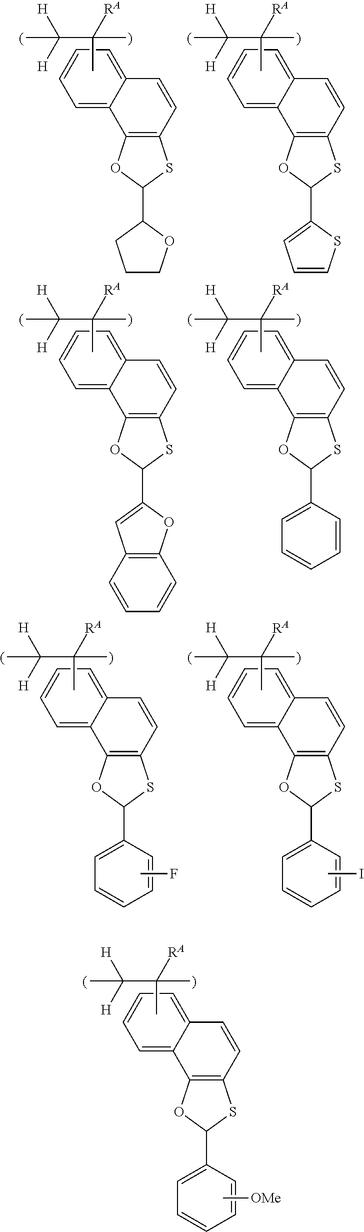

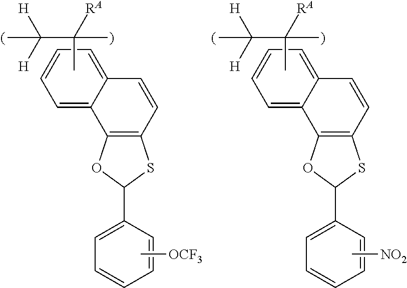

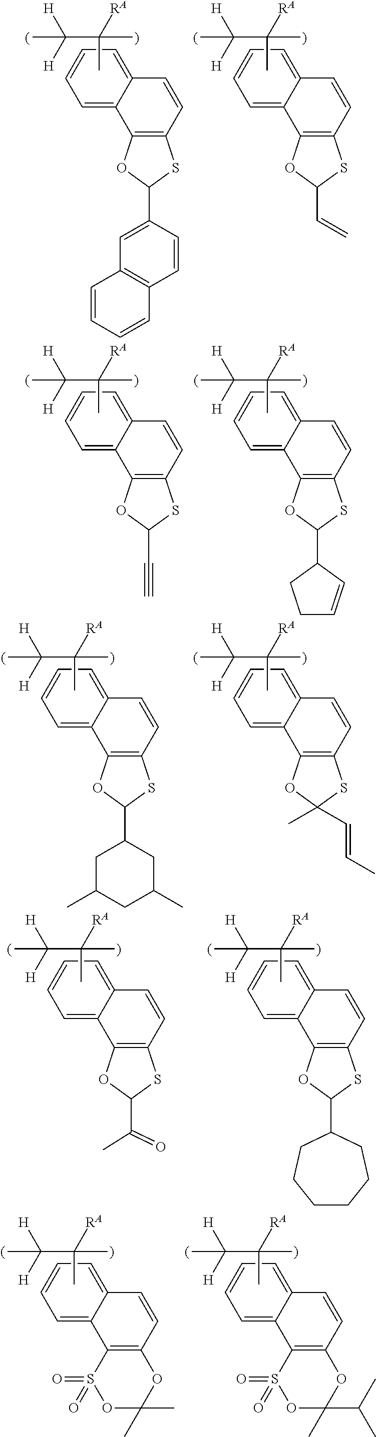

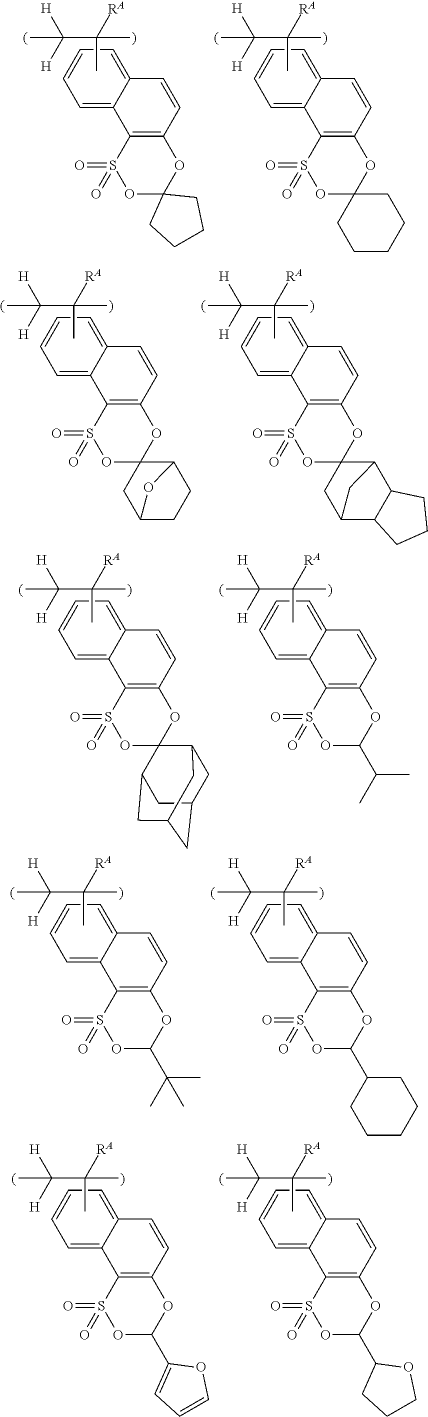

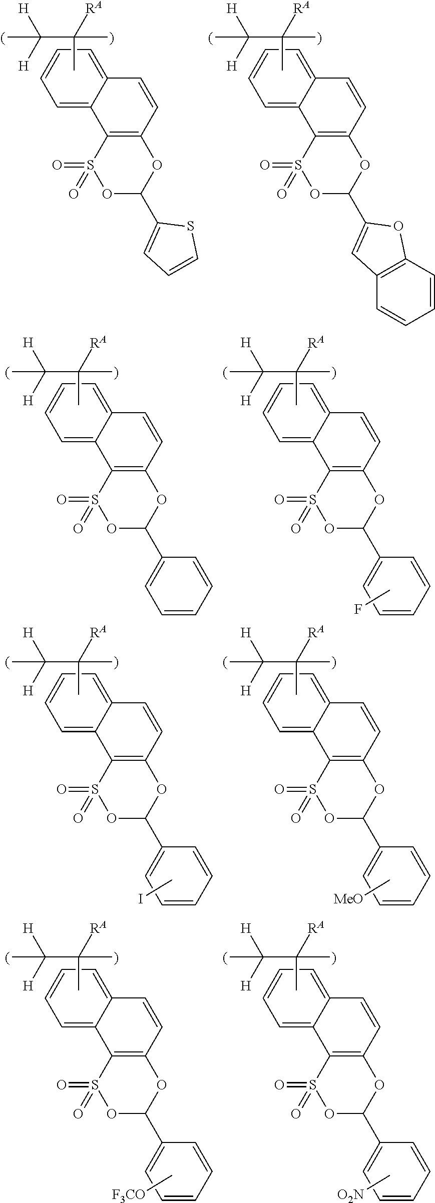

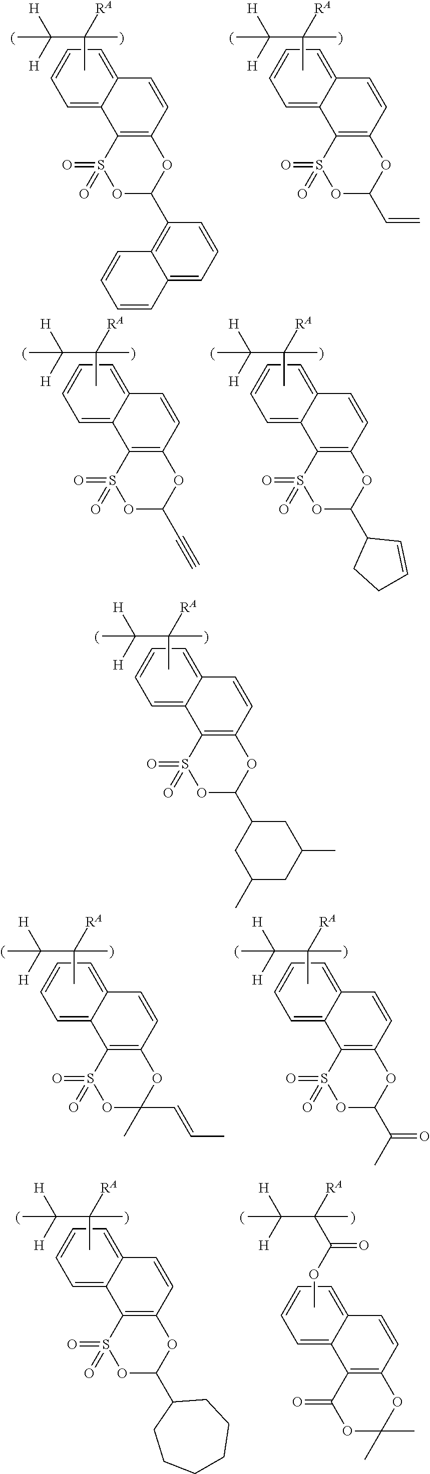

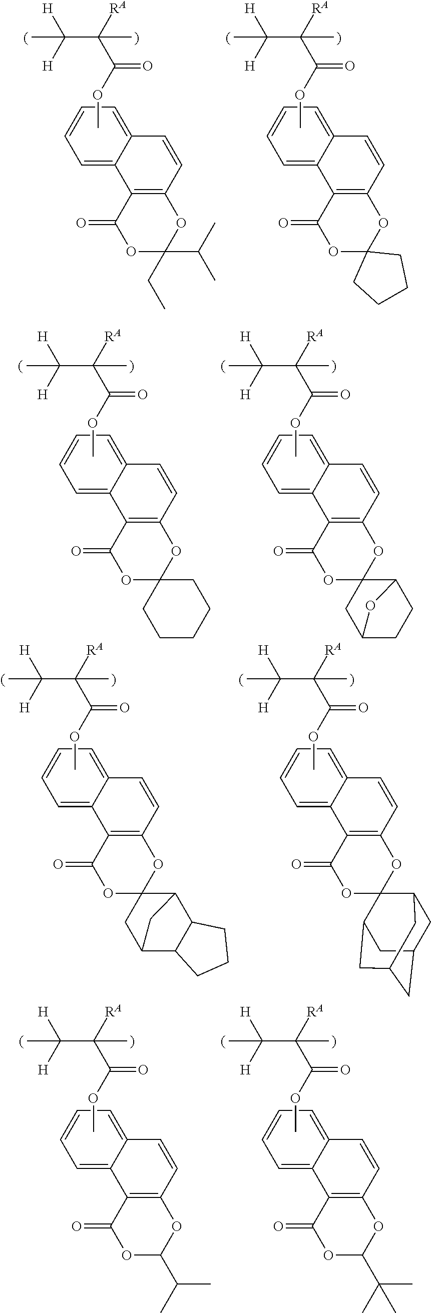

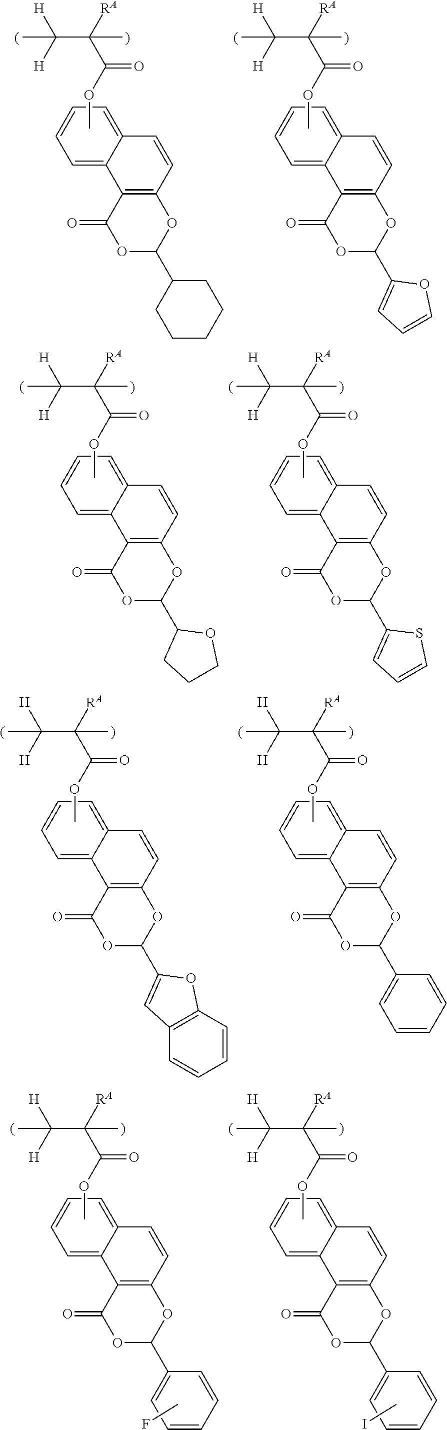

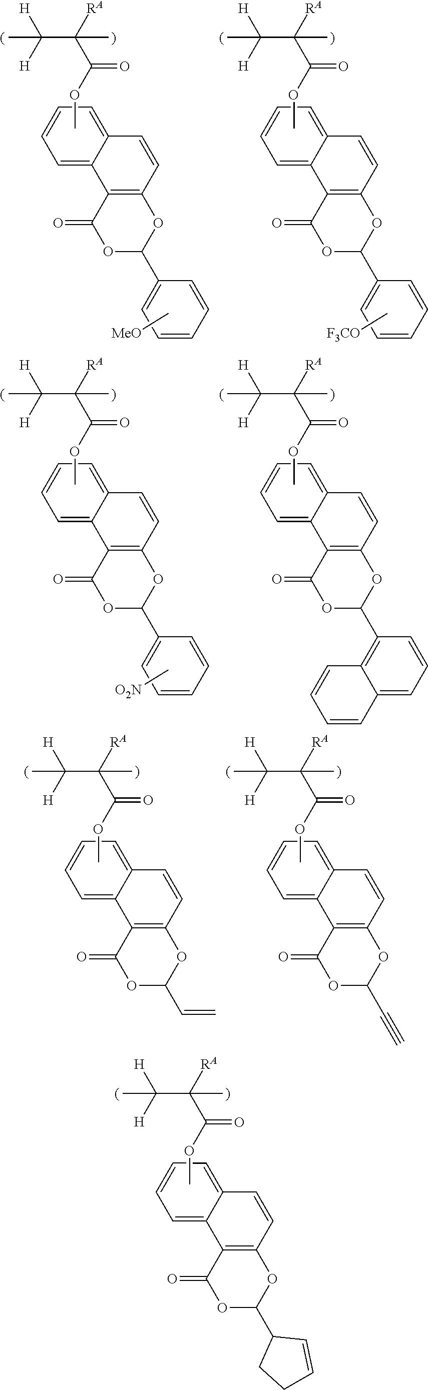

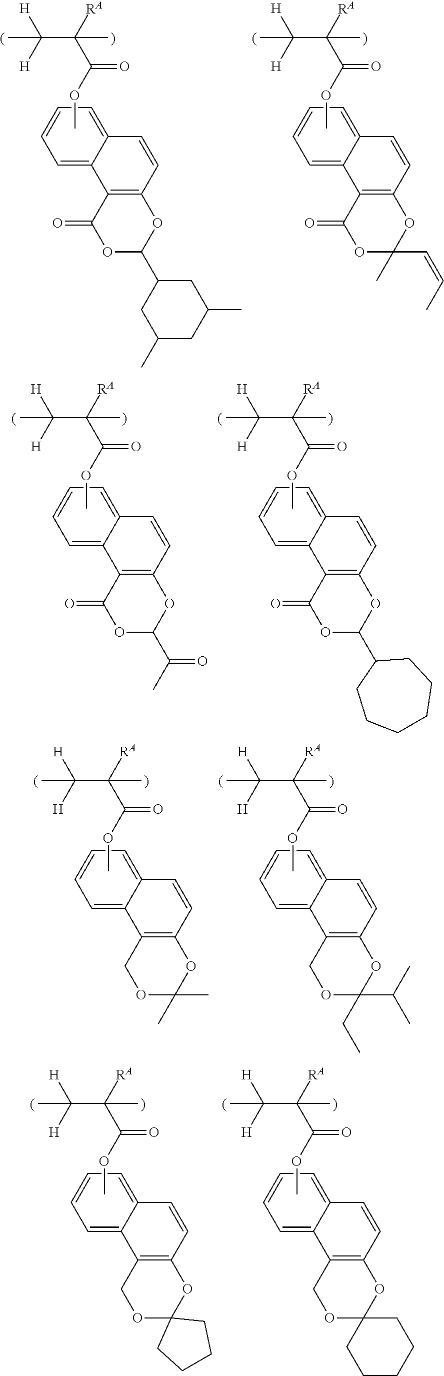

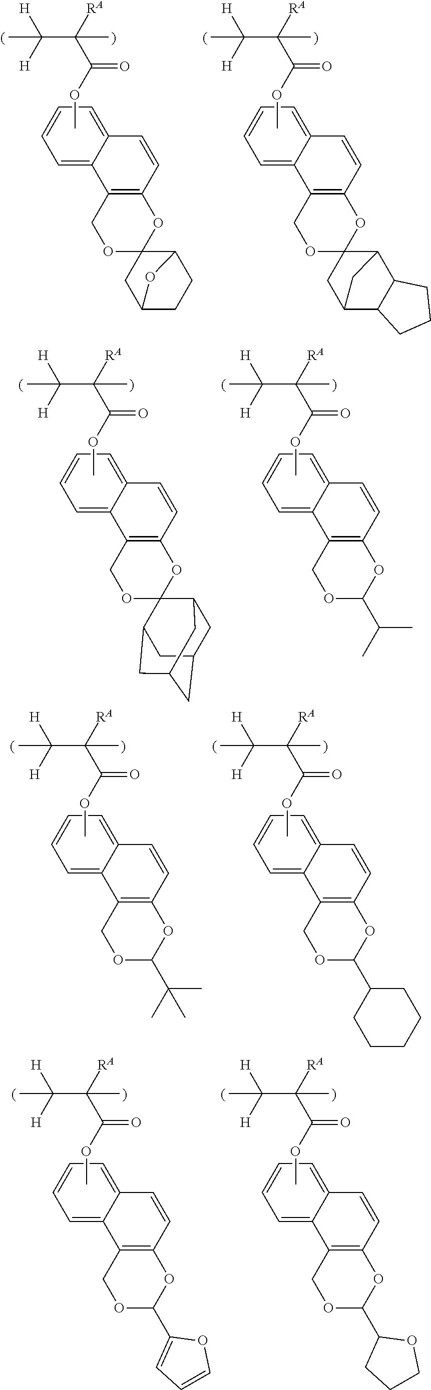

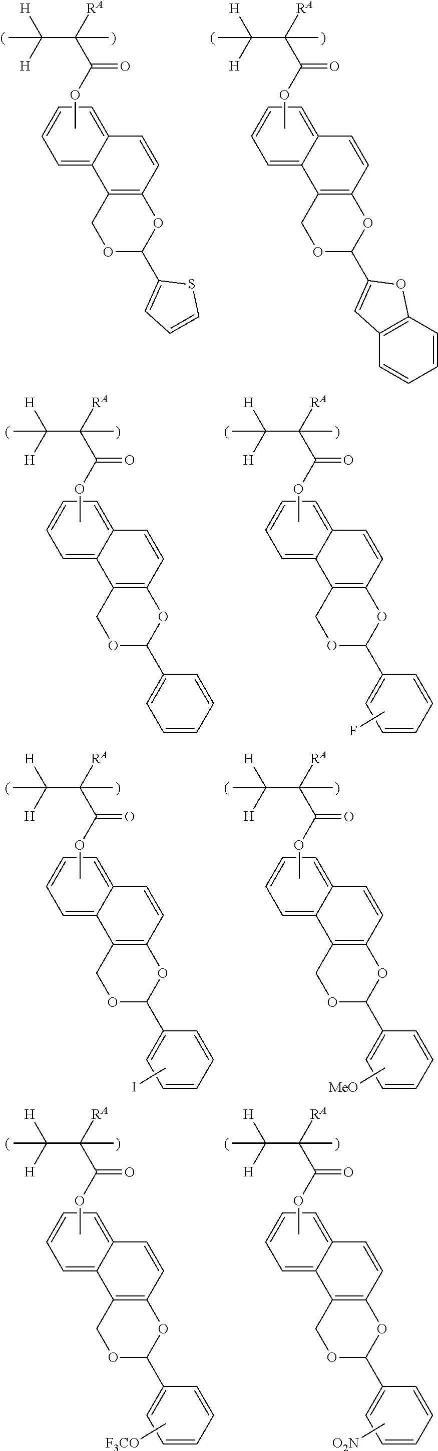

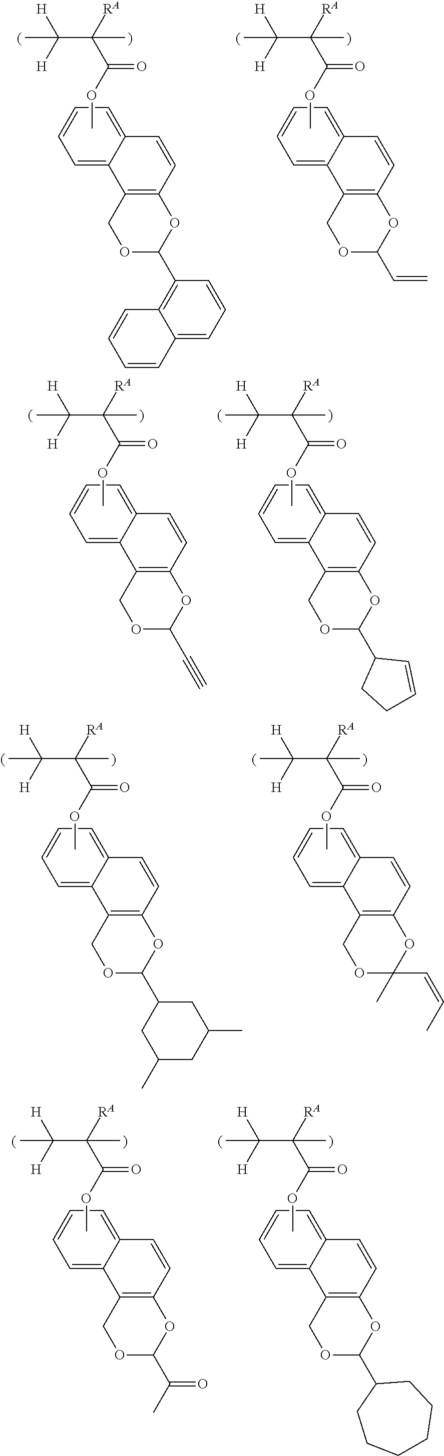

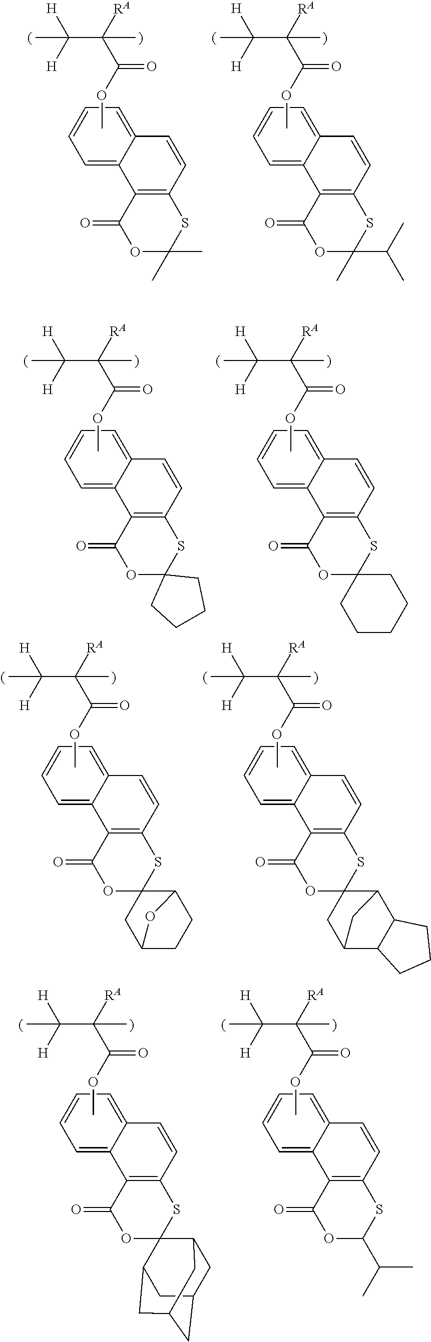

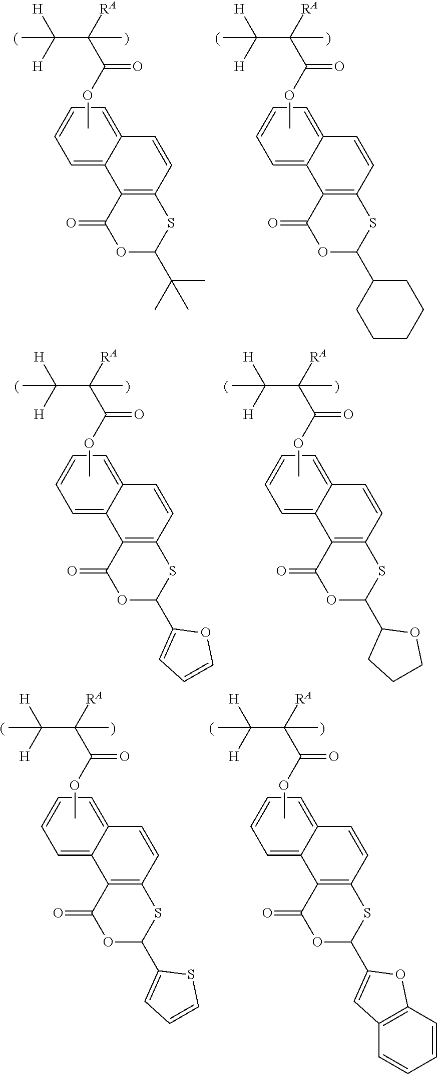

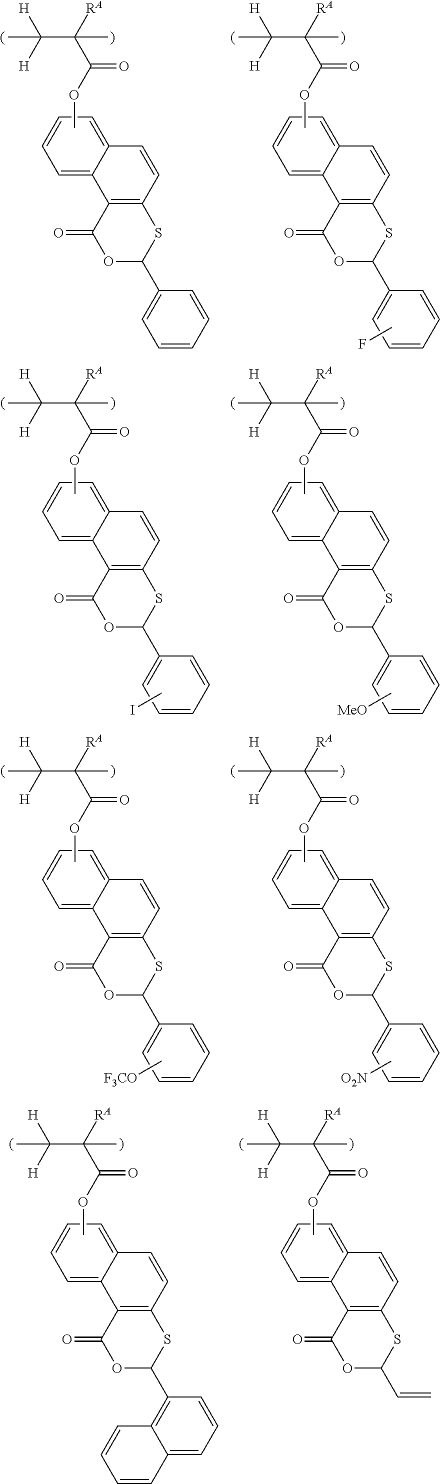

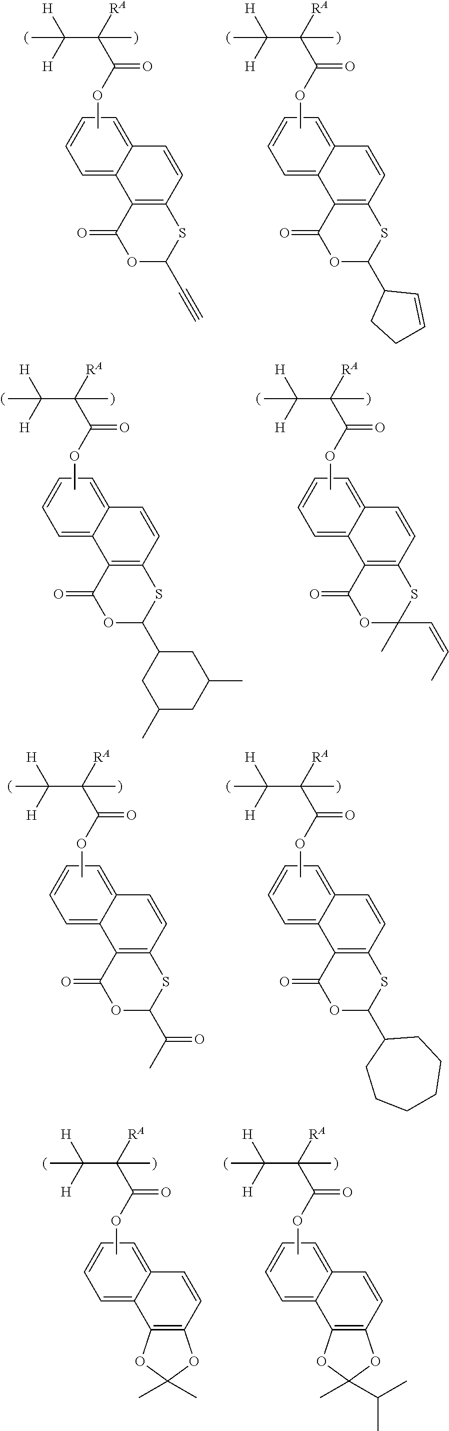

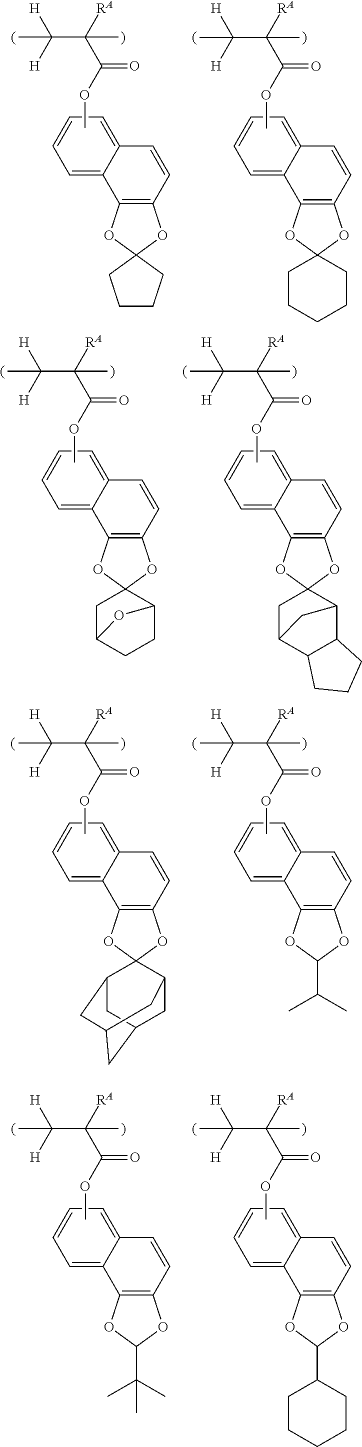

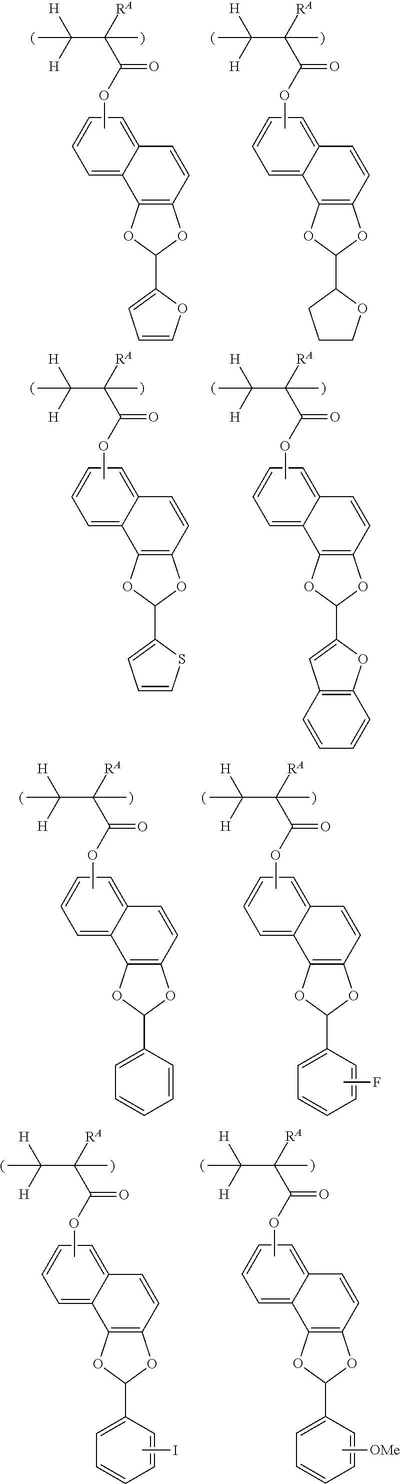

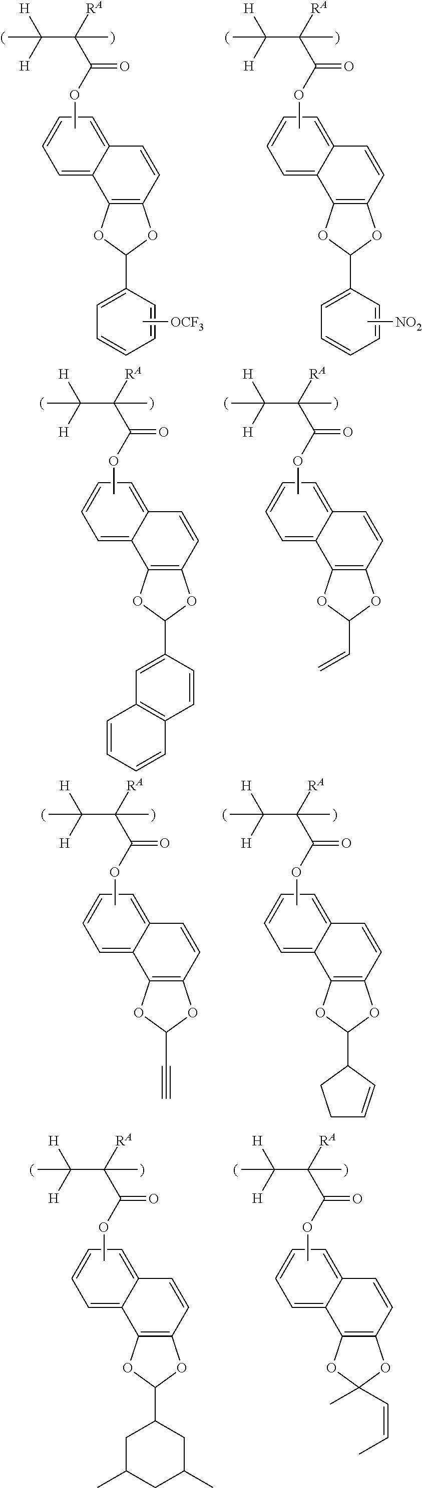

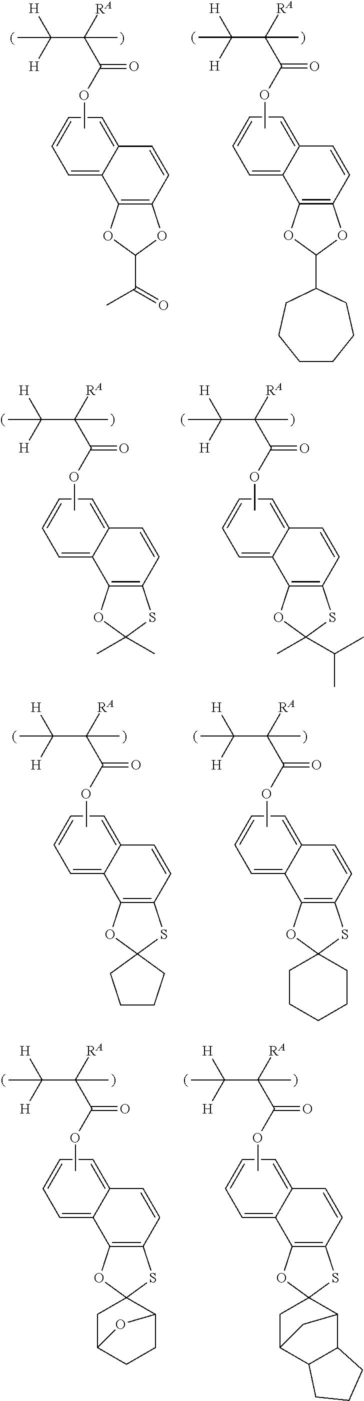

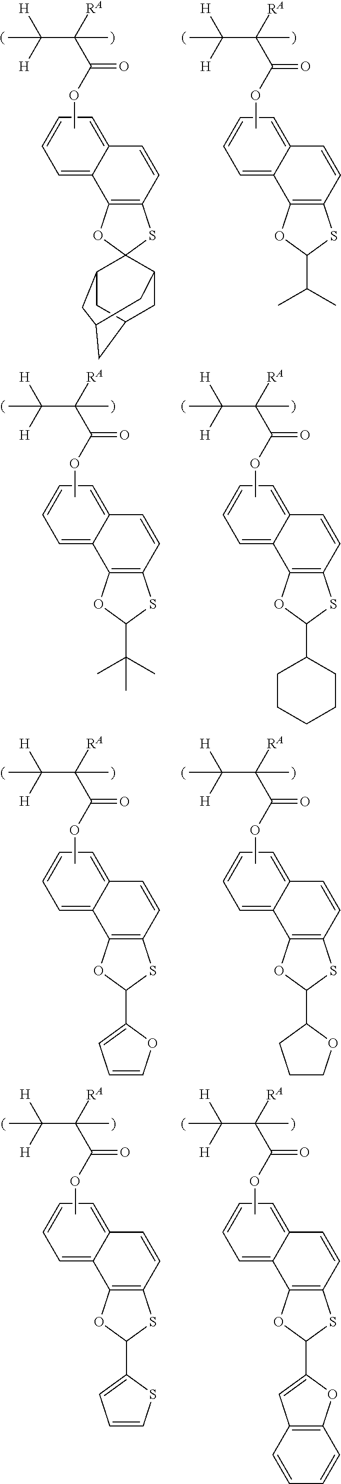

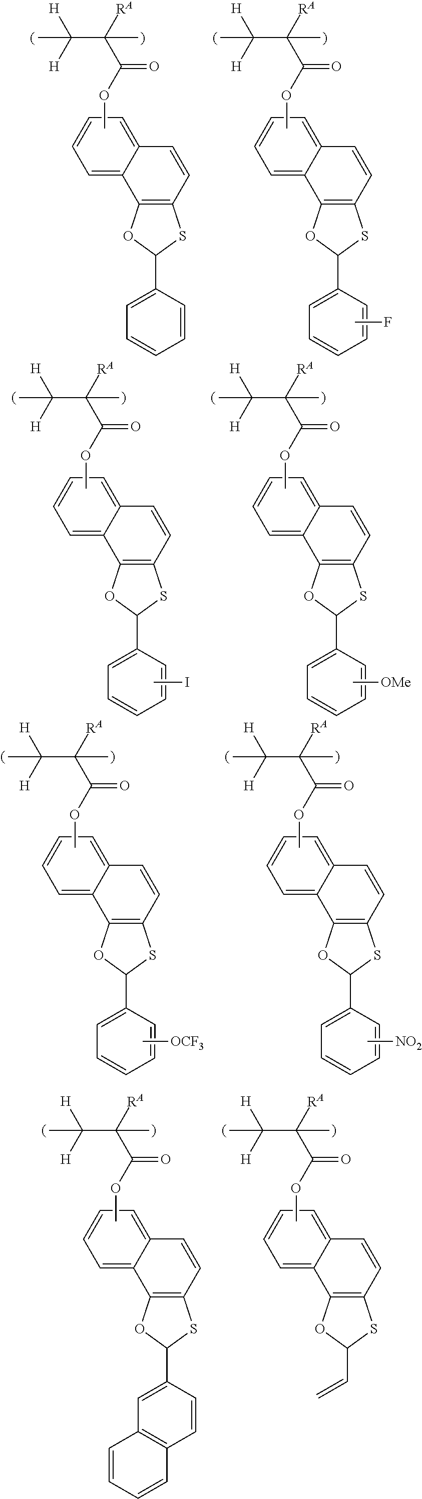

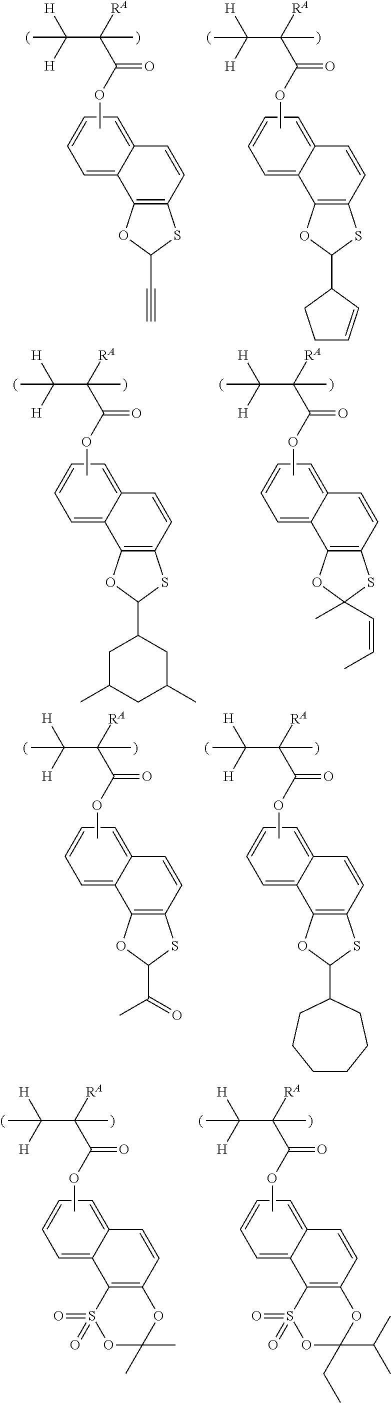

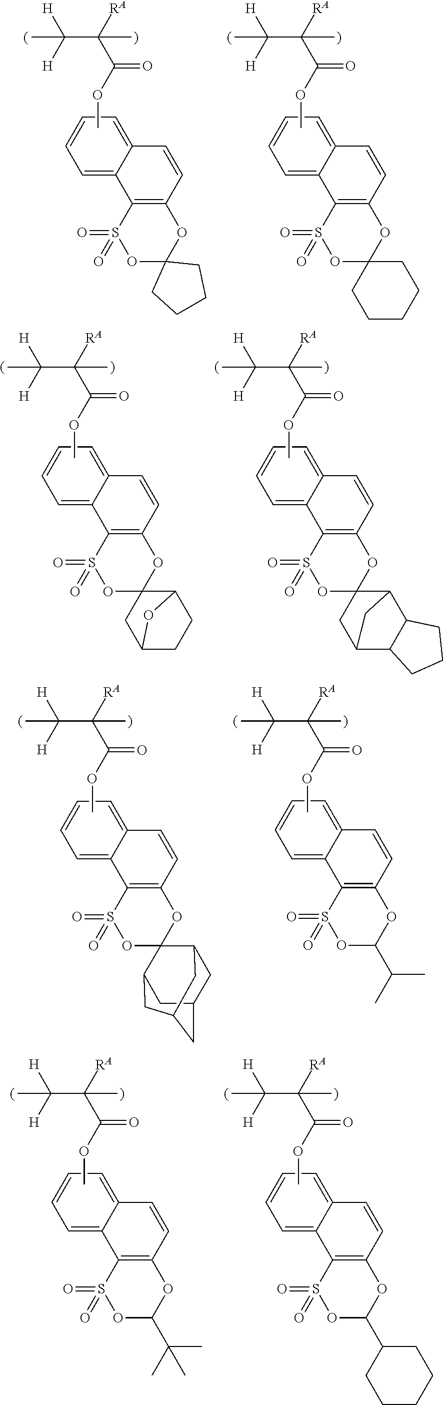

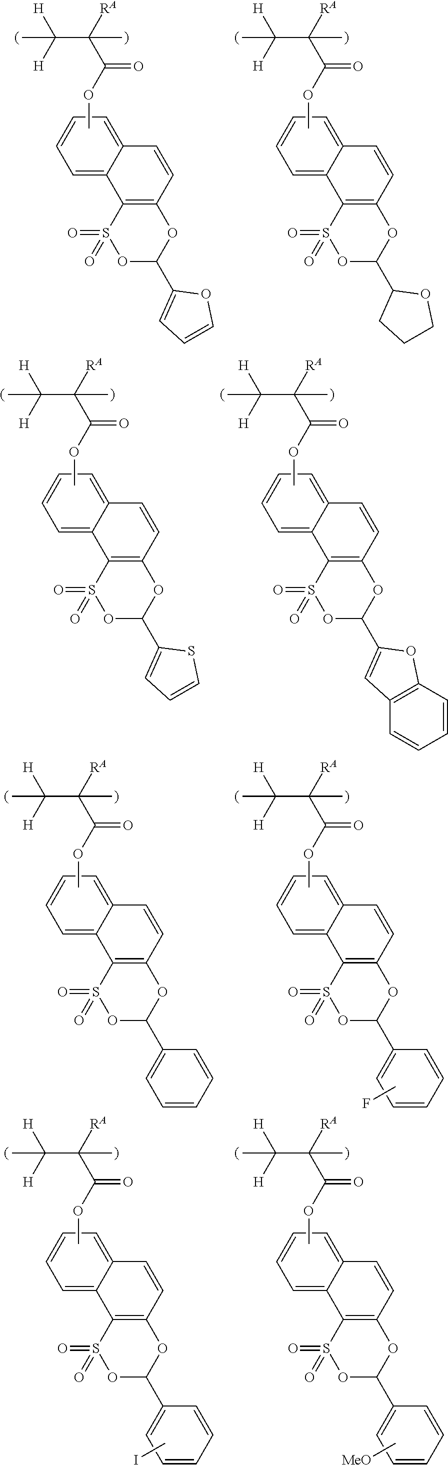

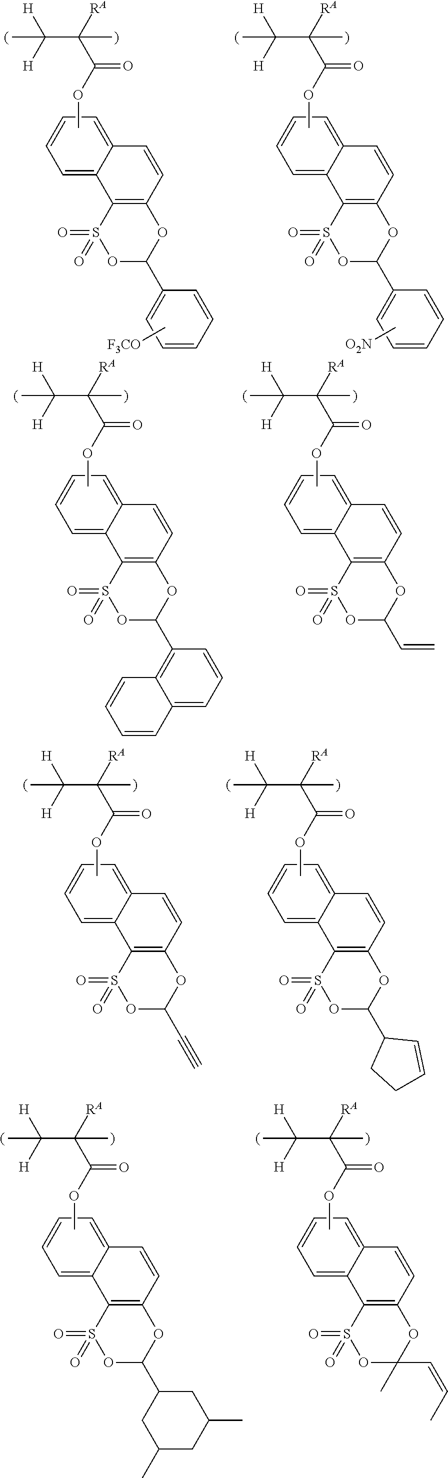

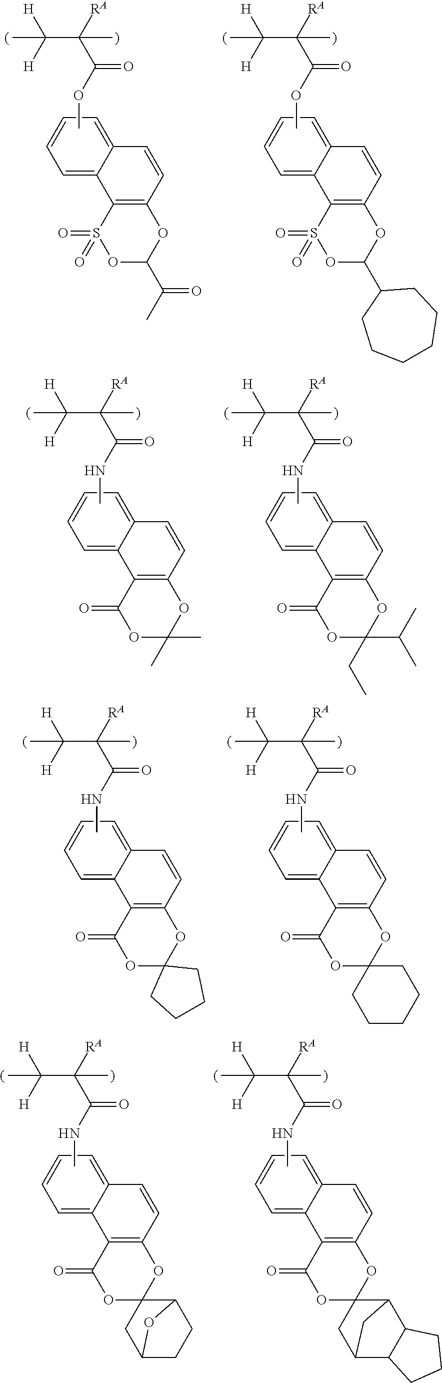

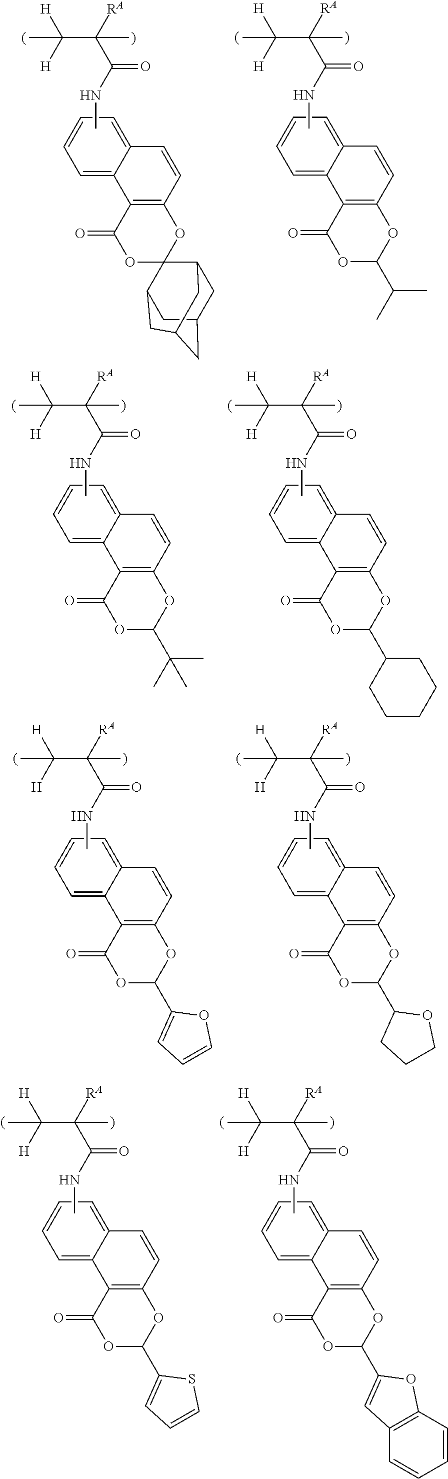

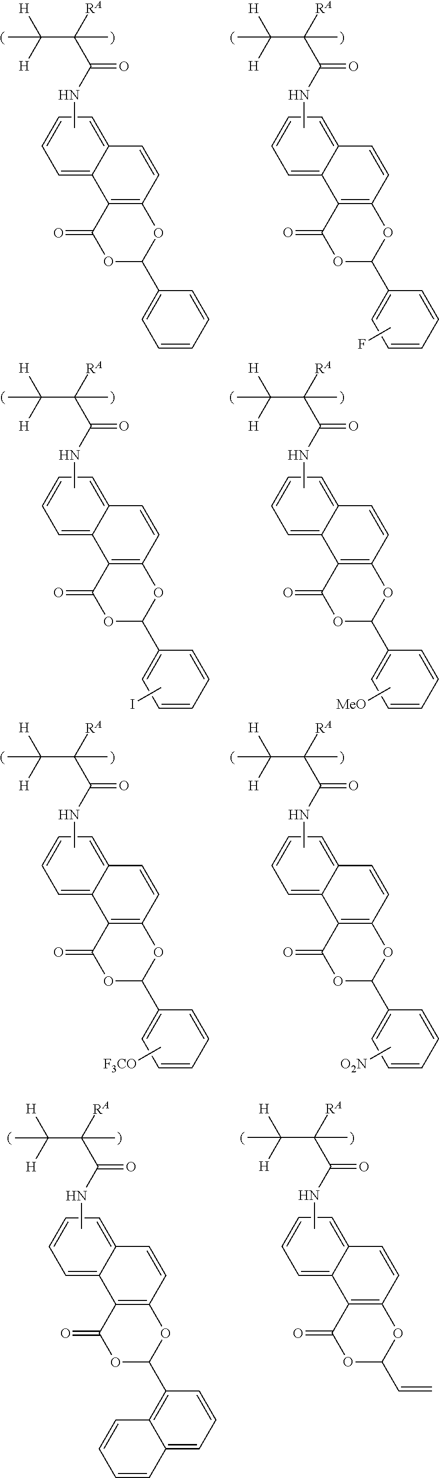

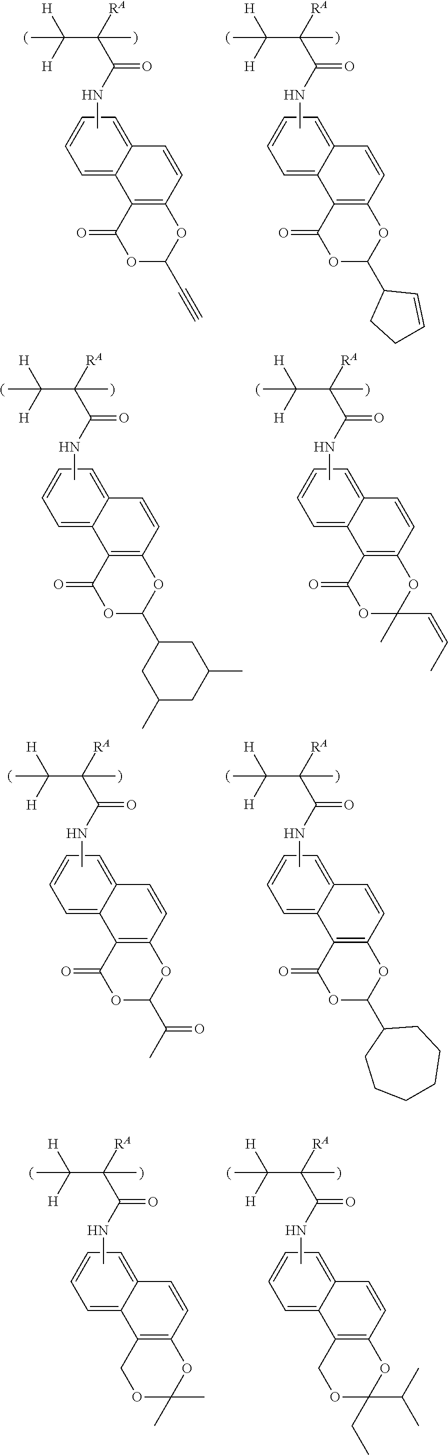

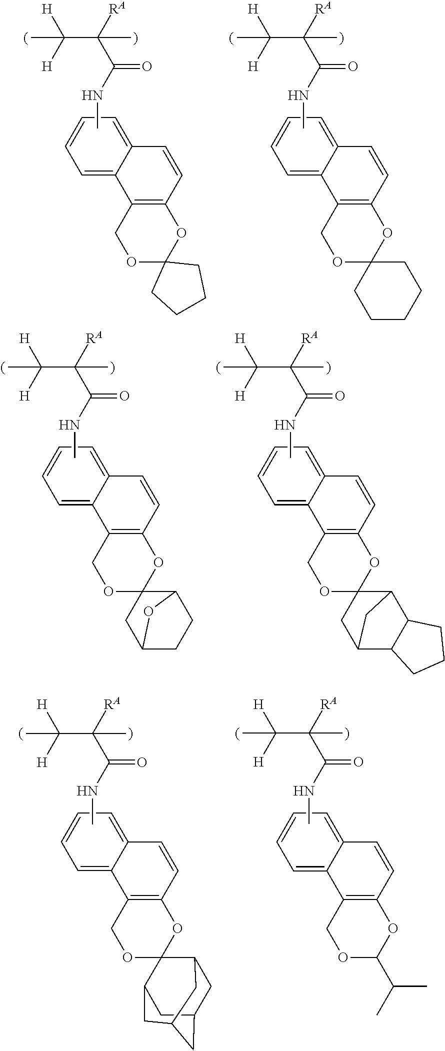

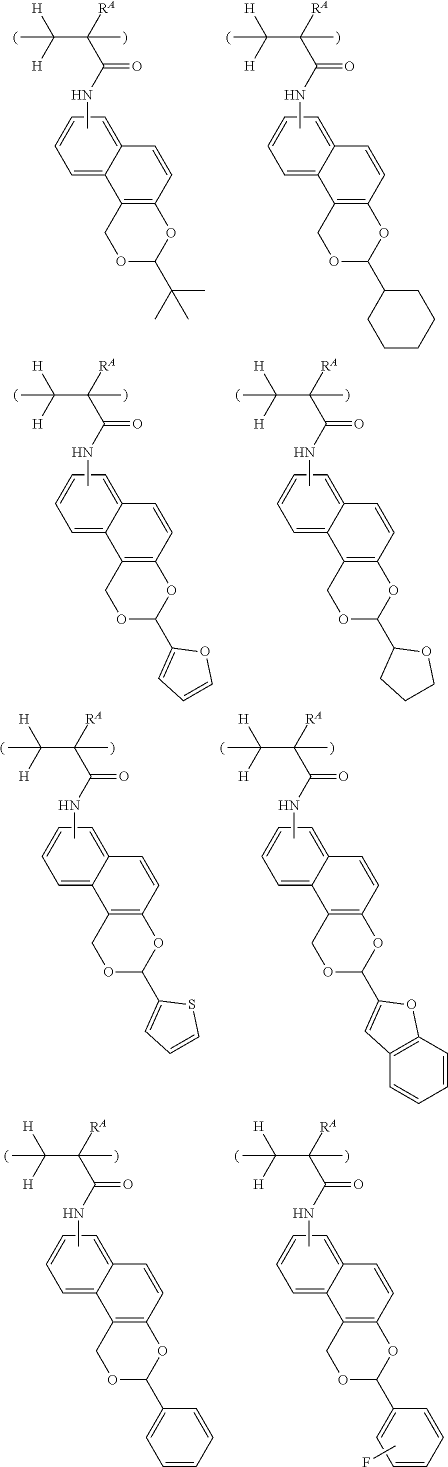

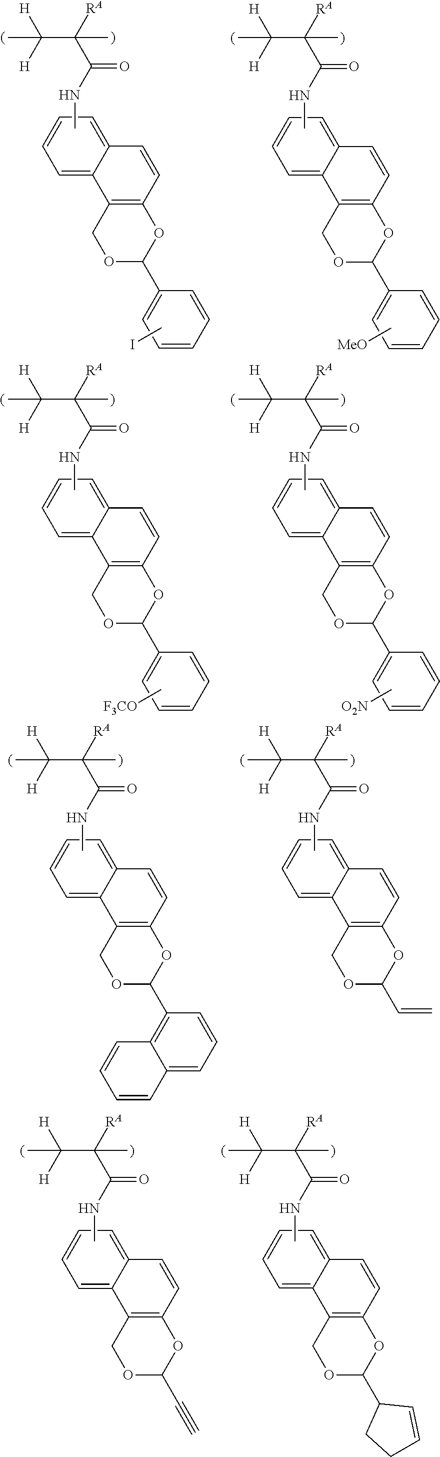

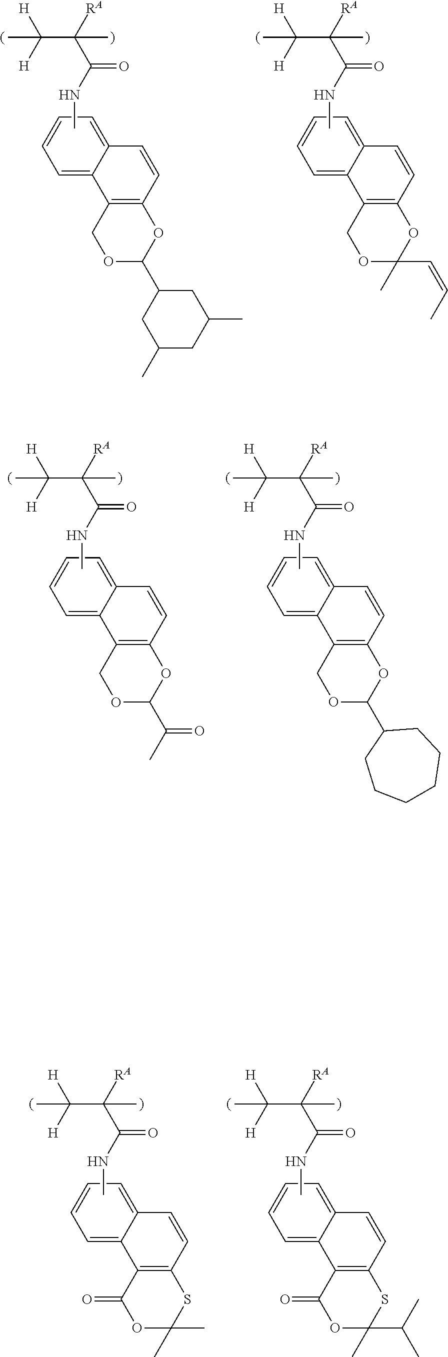

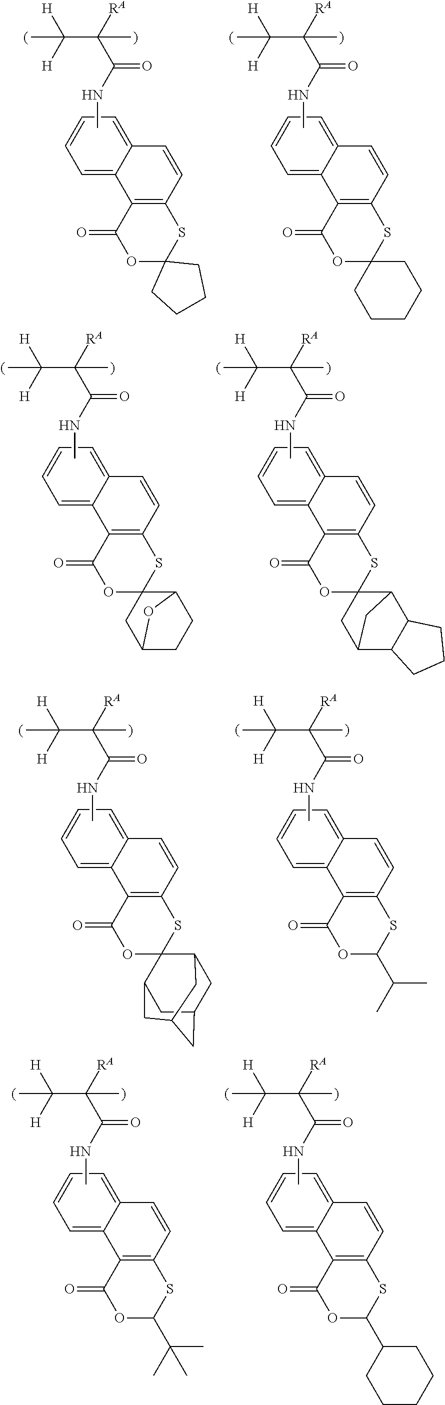

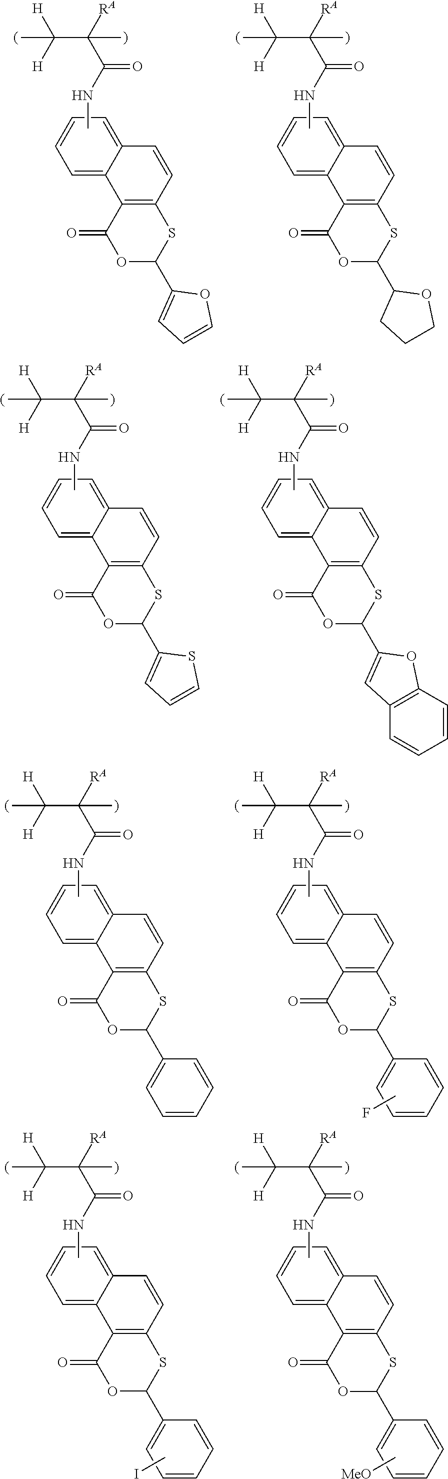

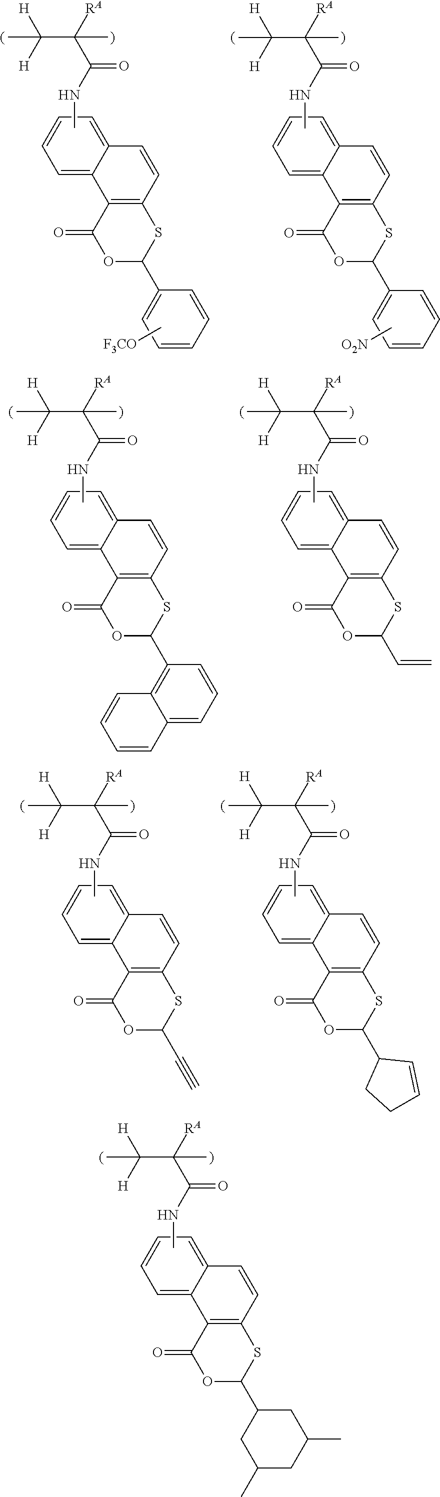

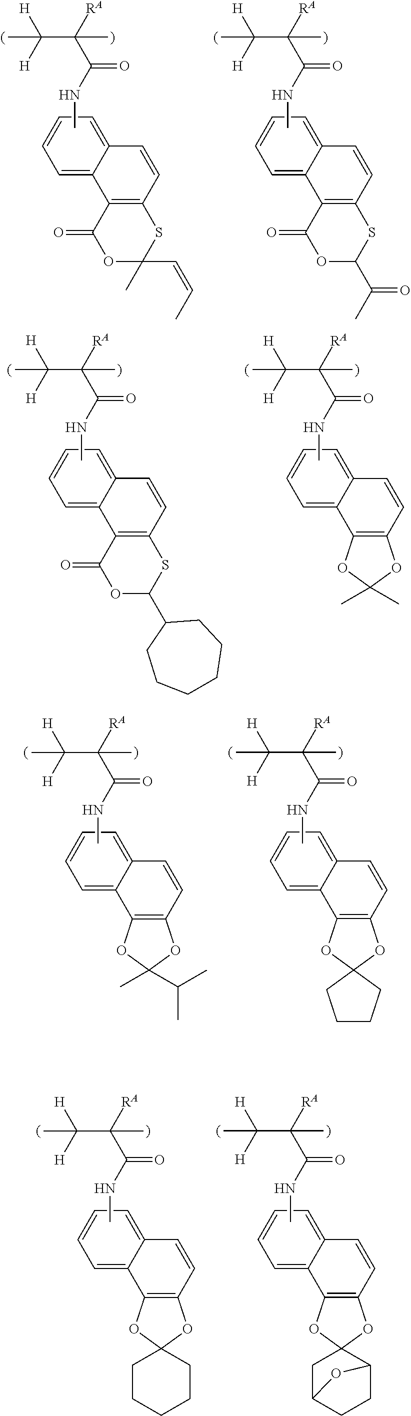

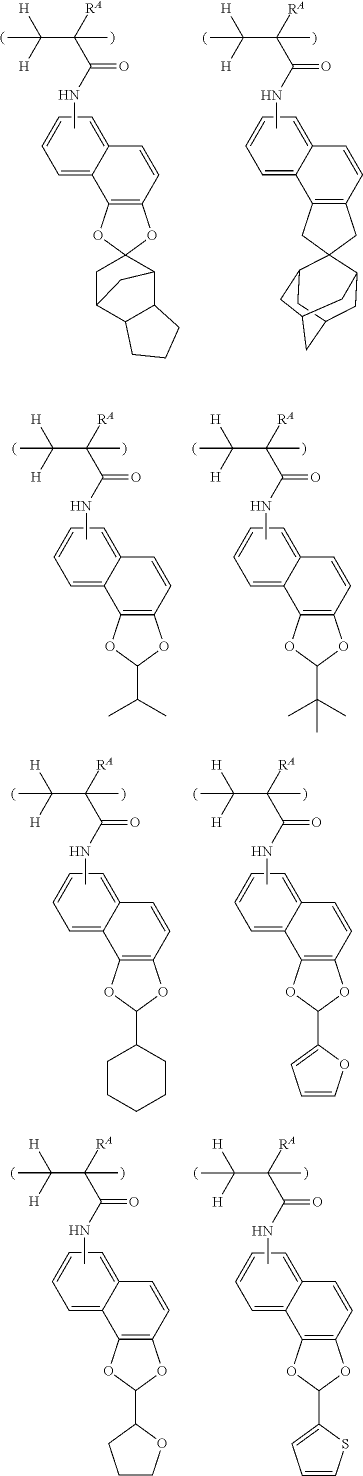

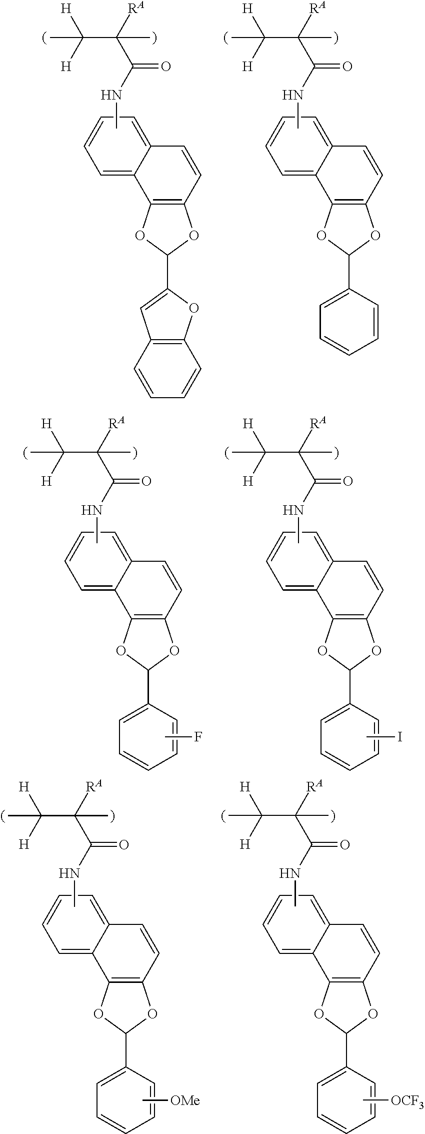

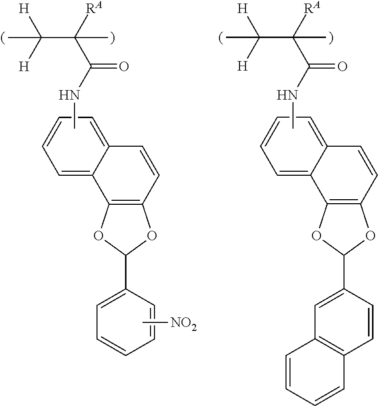

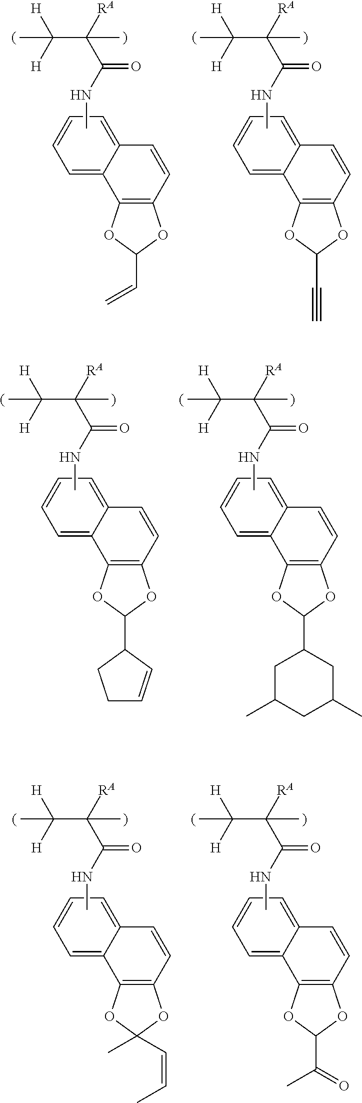

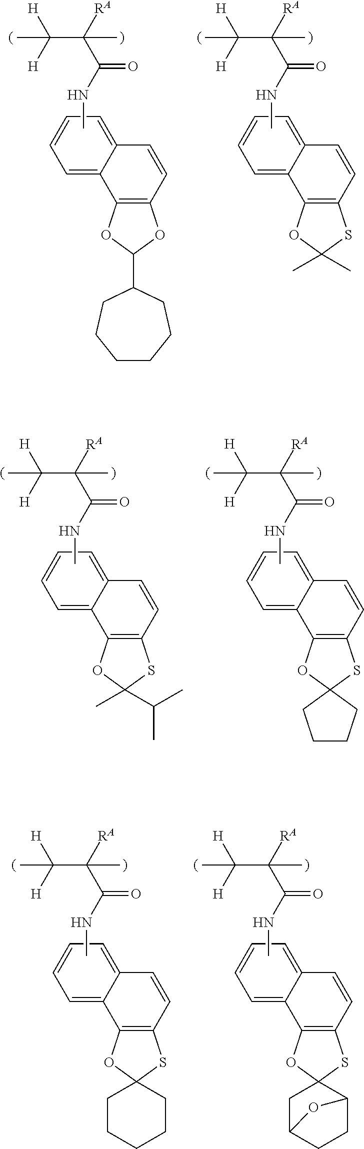

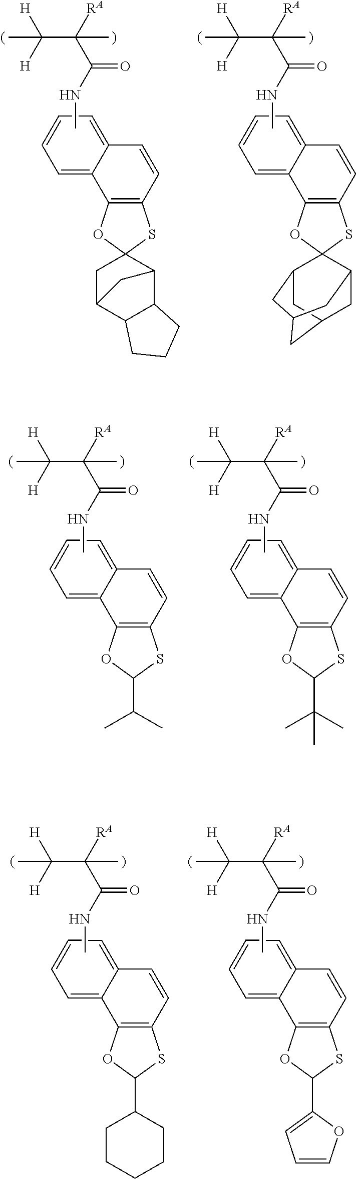

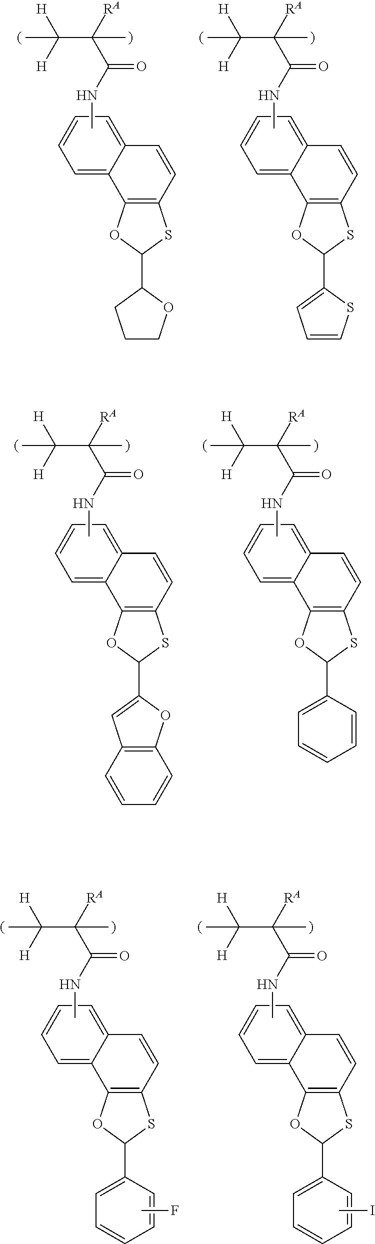

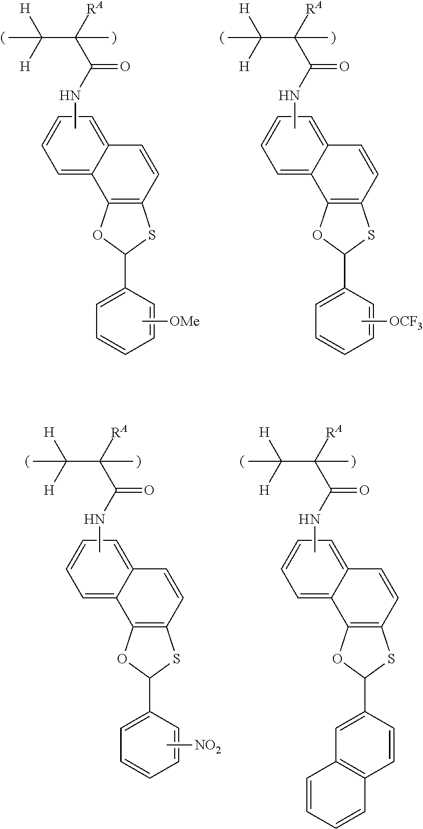

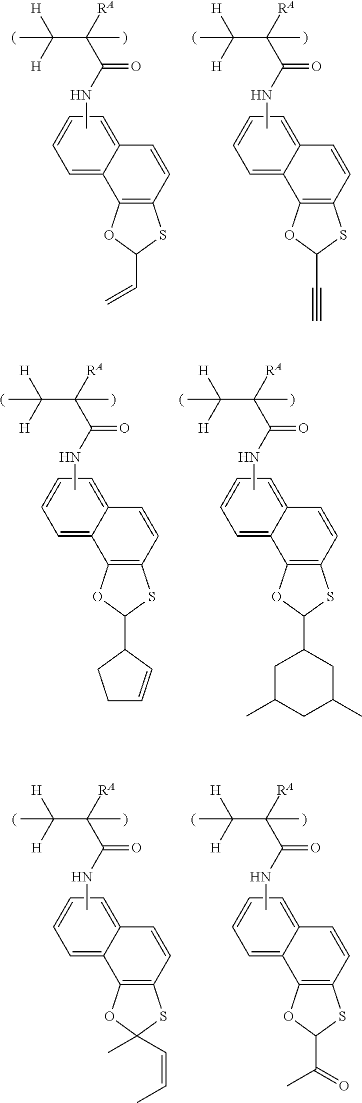

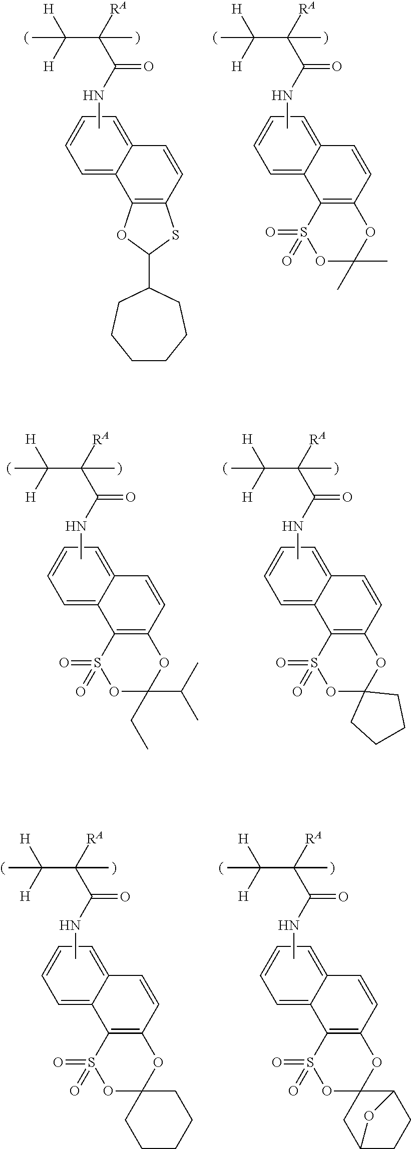

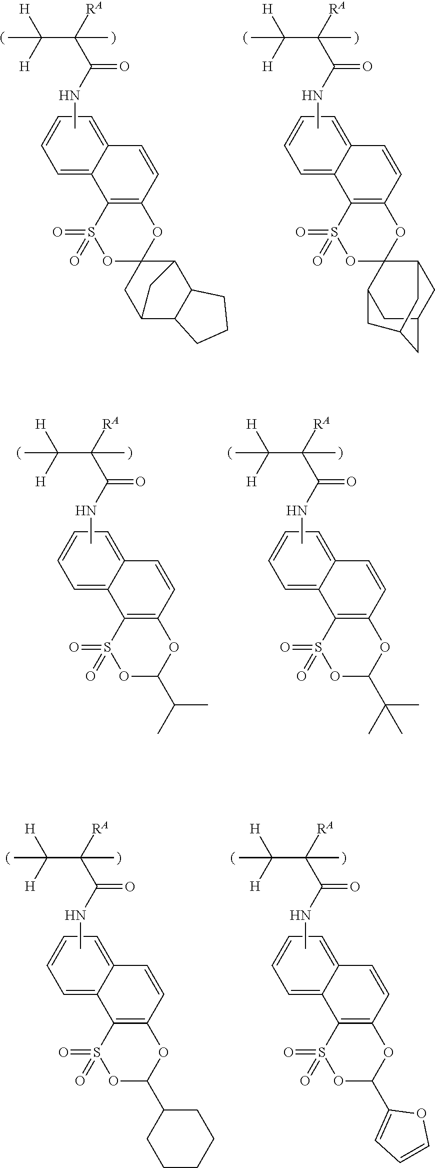

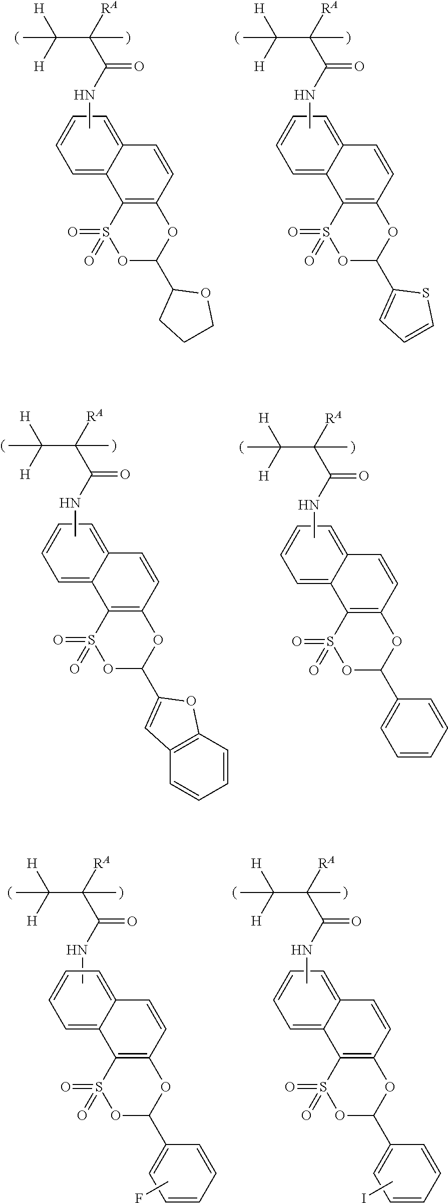

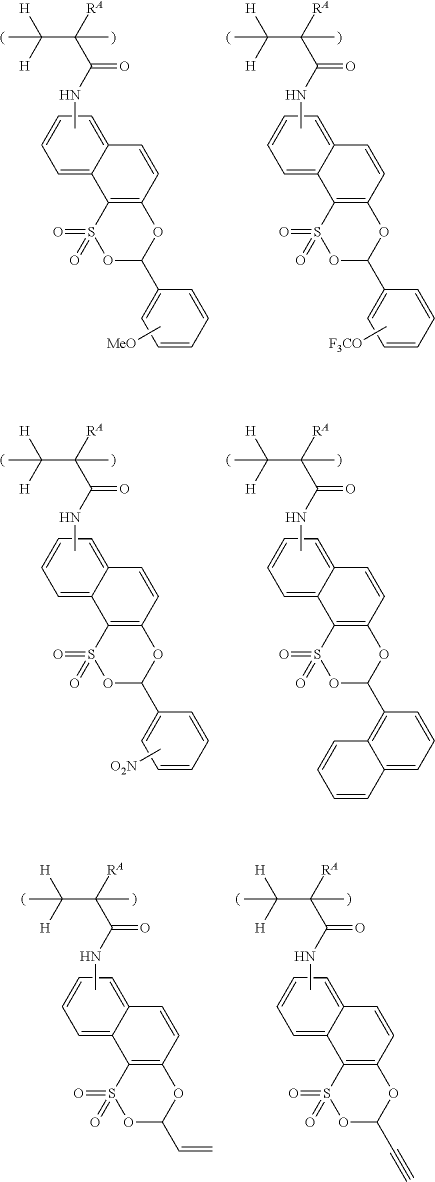

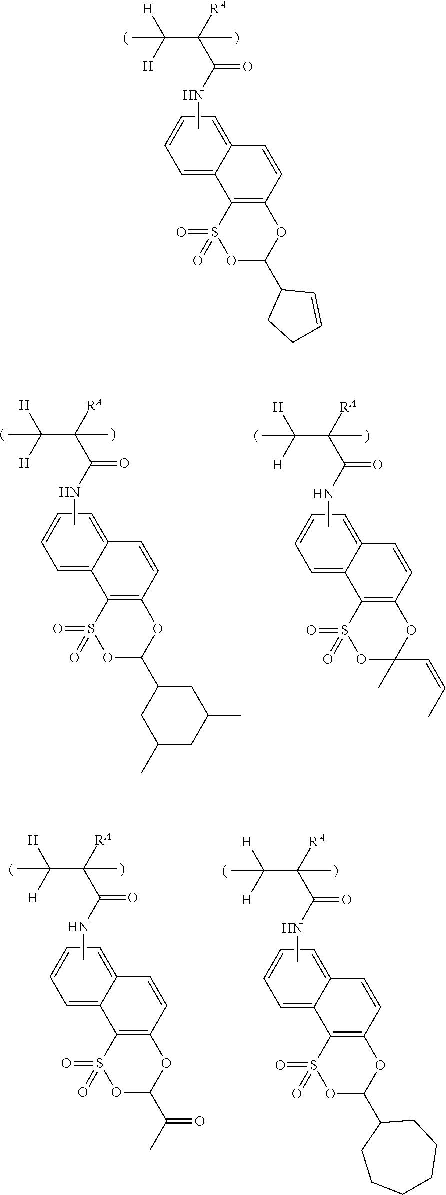

The base polymer preferably further comprise at least one selected from a repeat unit having the formula (b1)(hereinafter, the repeat unit is also referred to as a repeat unit b1) and a repeat unit having the formula (b2)(hereinafter, the repeat unit is also referred to as a repeat unit b2).

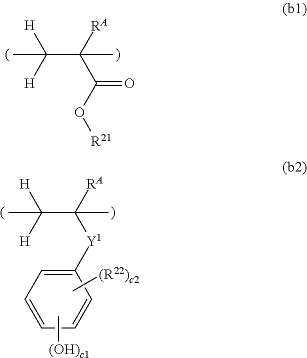

In formulae (b1) and (b2), R^{A} is each independently a hydrogen atom, a fluorine atom, a methyl group, or a trifluoromethyl group. Y¹ is a single bond or *—C(═O)—O—. * designates a point of attachment to the carbon atom in the backbone. R²¹ is a hydrogen atom or a C₁-C₂₀ group having at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—). R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. c1 is 1, 2, 3, or 4. c2 is 0, 1, 2, 3, or 4. Provided that 1≤c1+c2≤5. When c2 is 2, 3, or 4, R²² may be identical or different.

Specific examples of the repeat unit b1 are shown below, but not limited thereto. Herein R^{A} is as defined above.

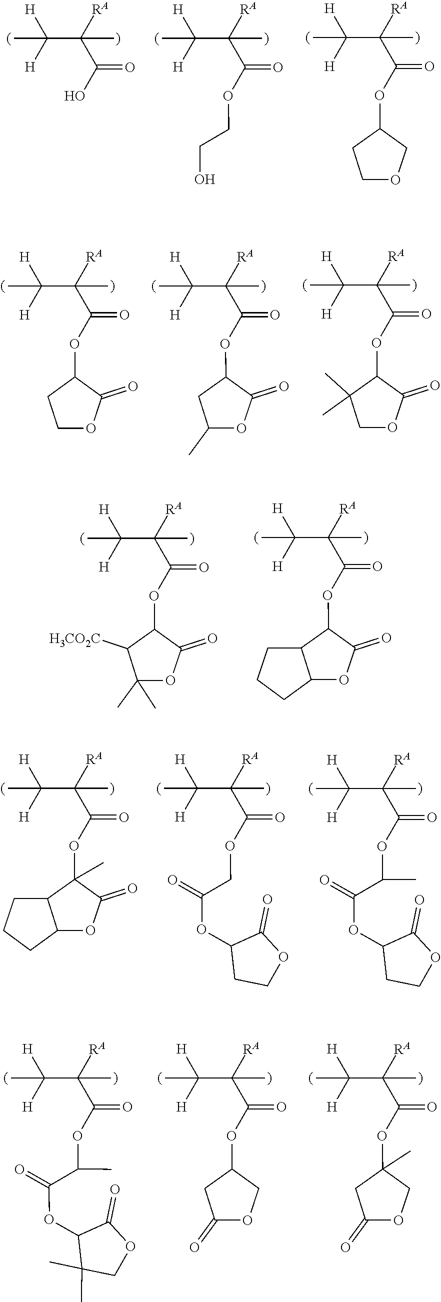

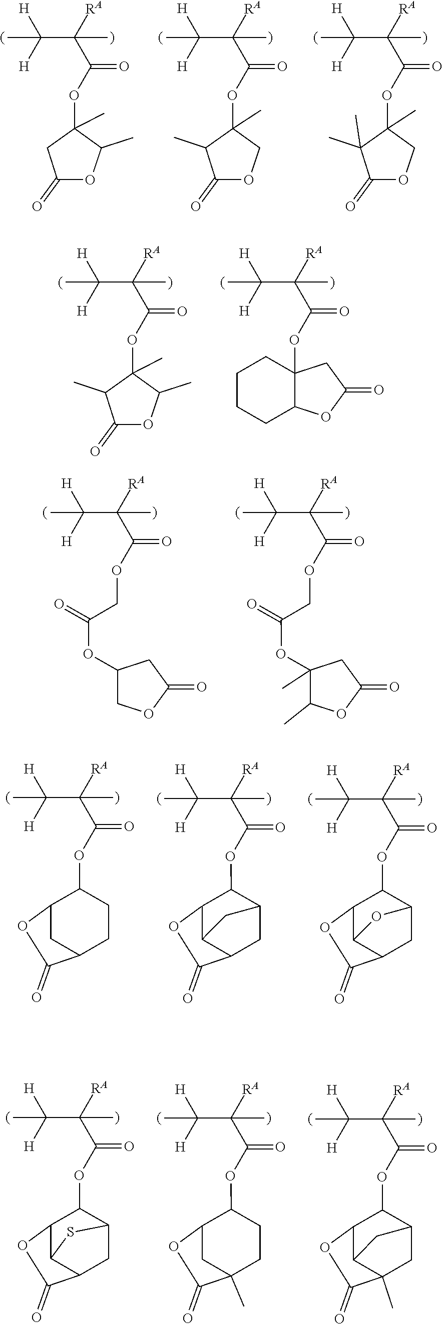

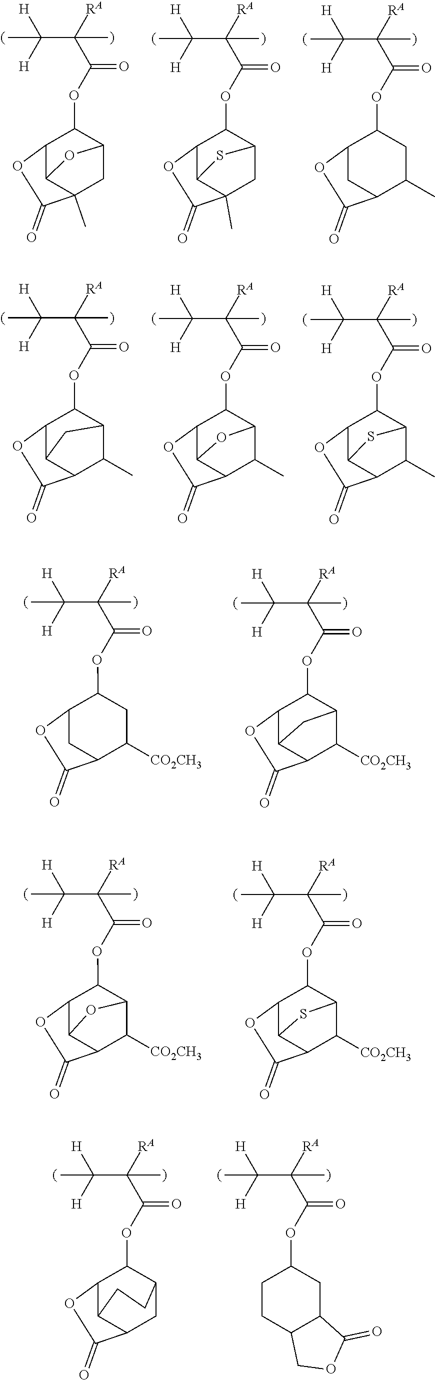

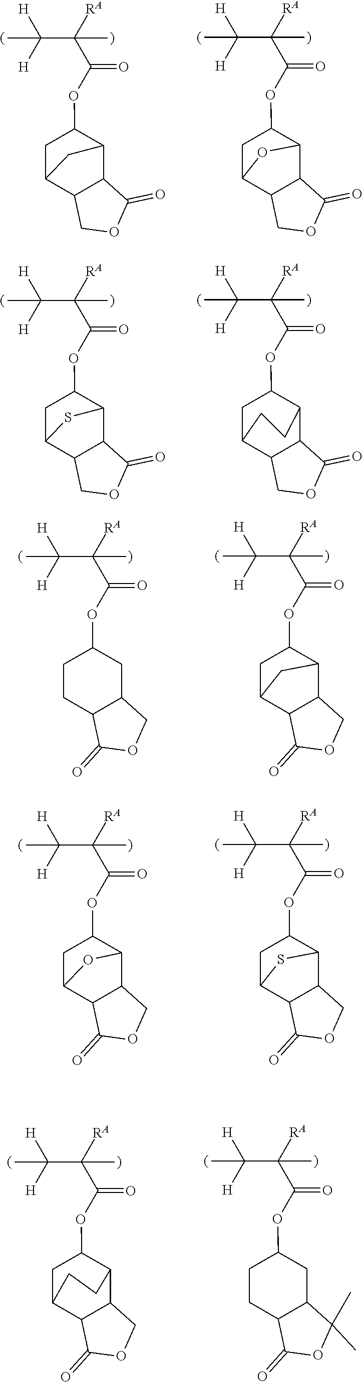

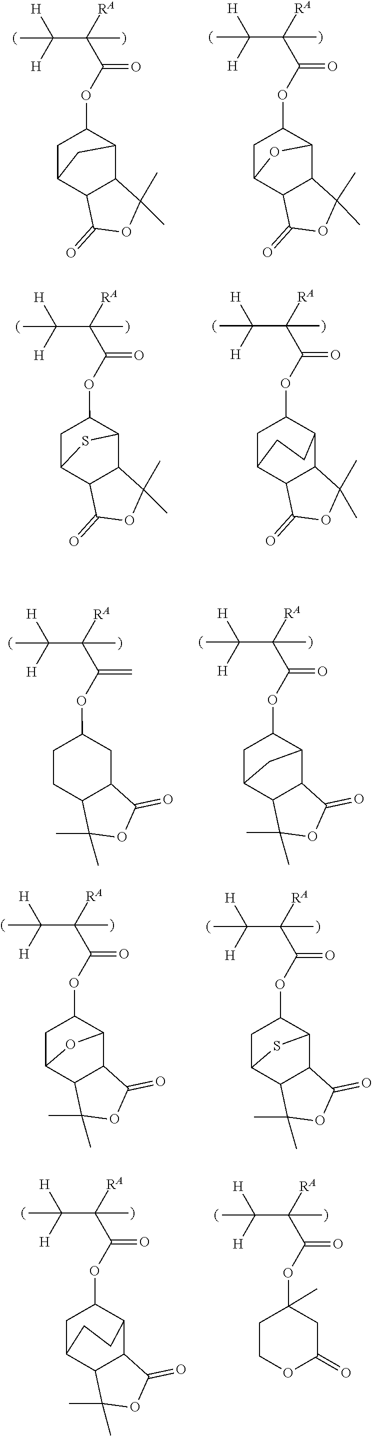

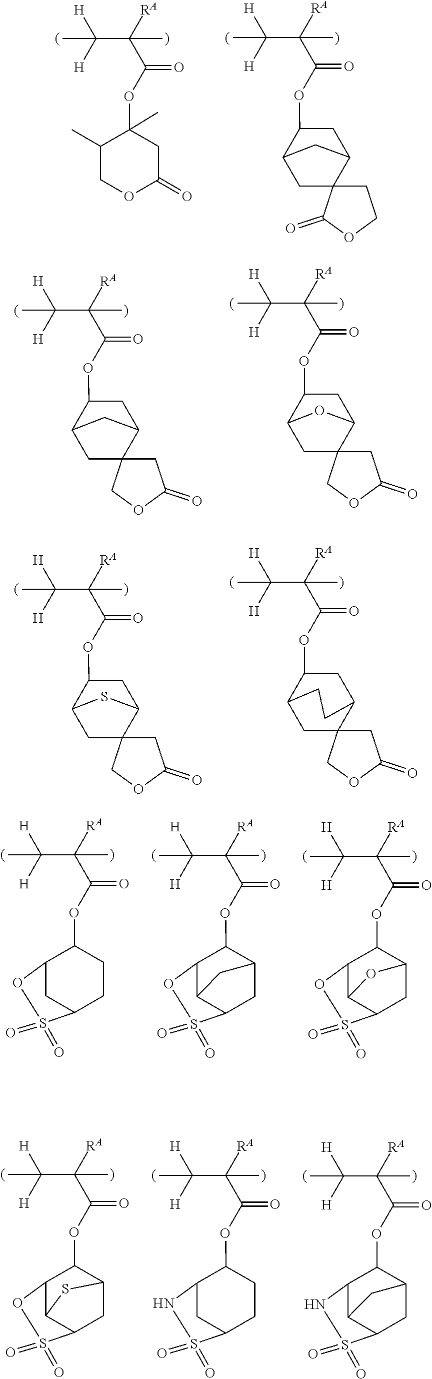

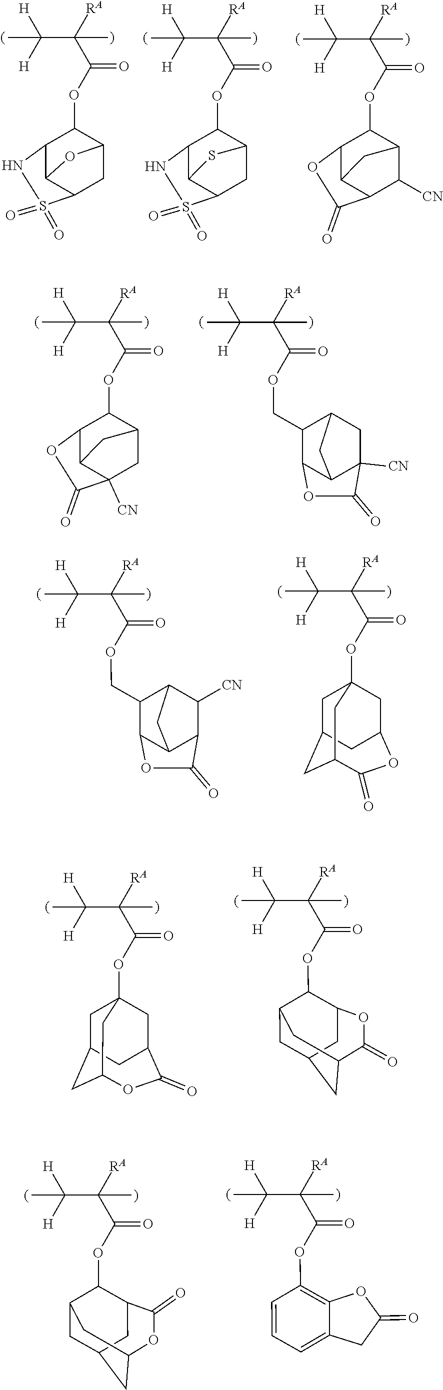

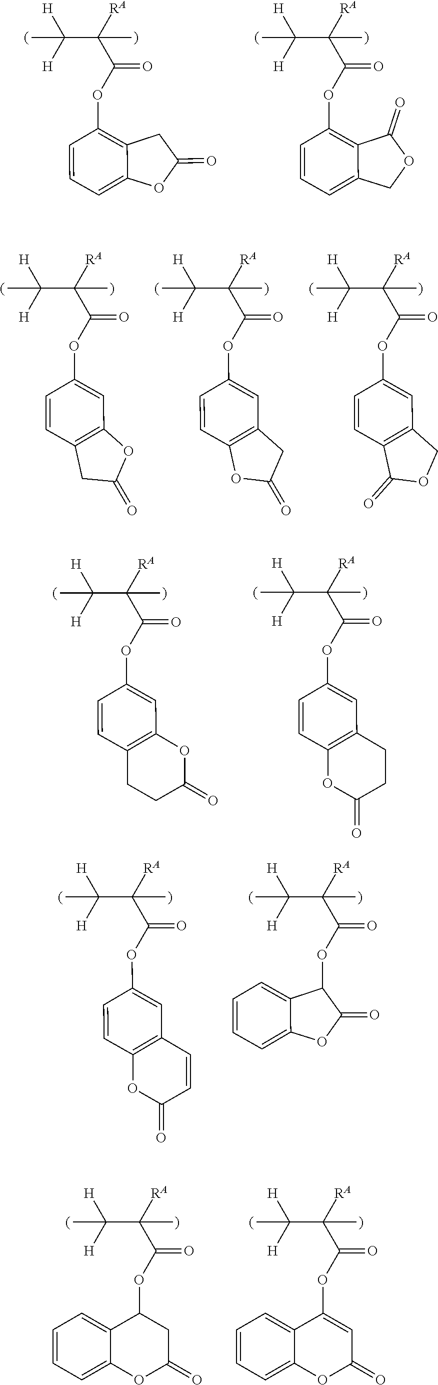

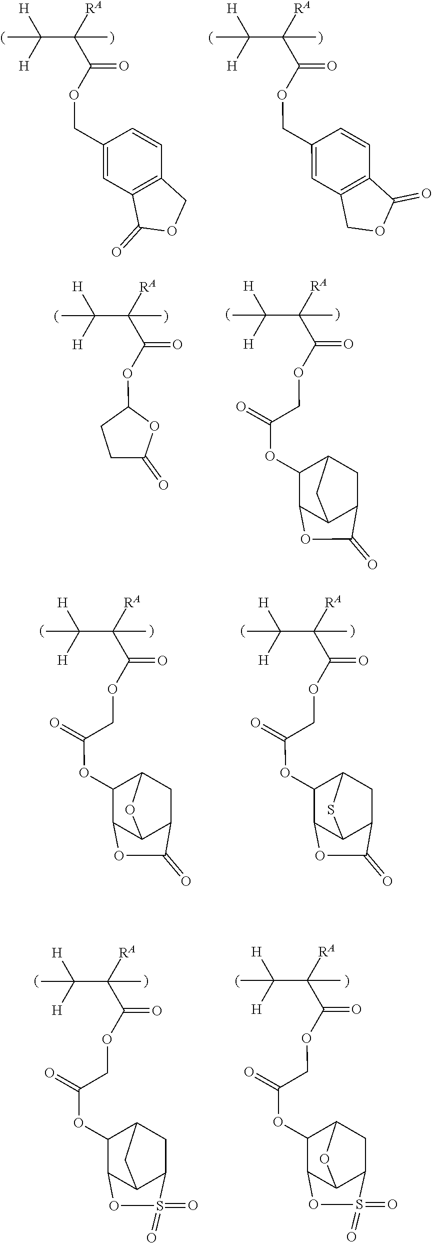

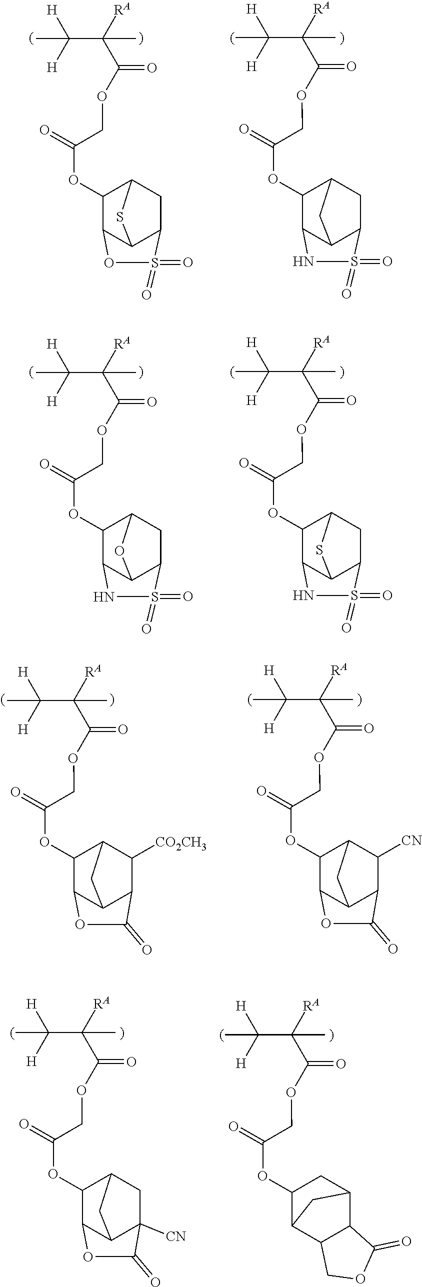

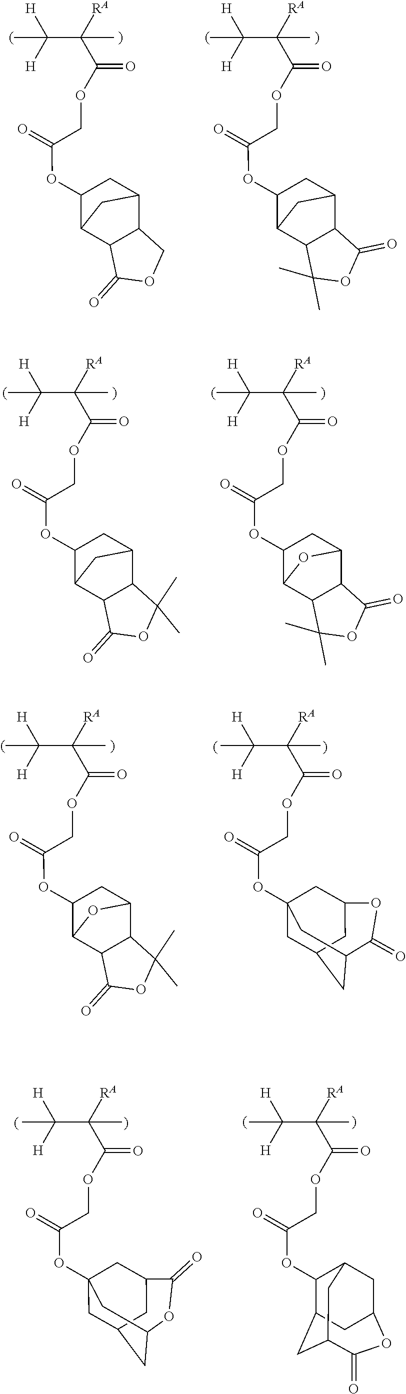

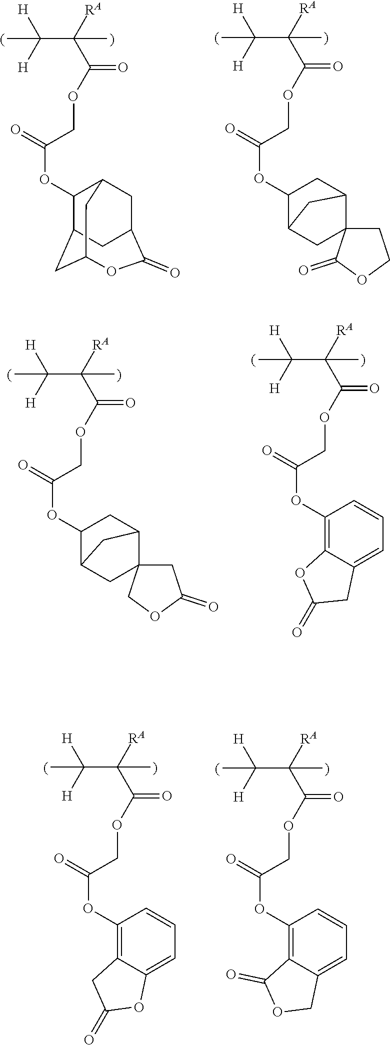

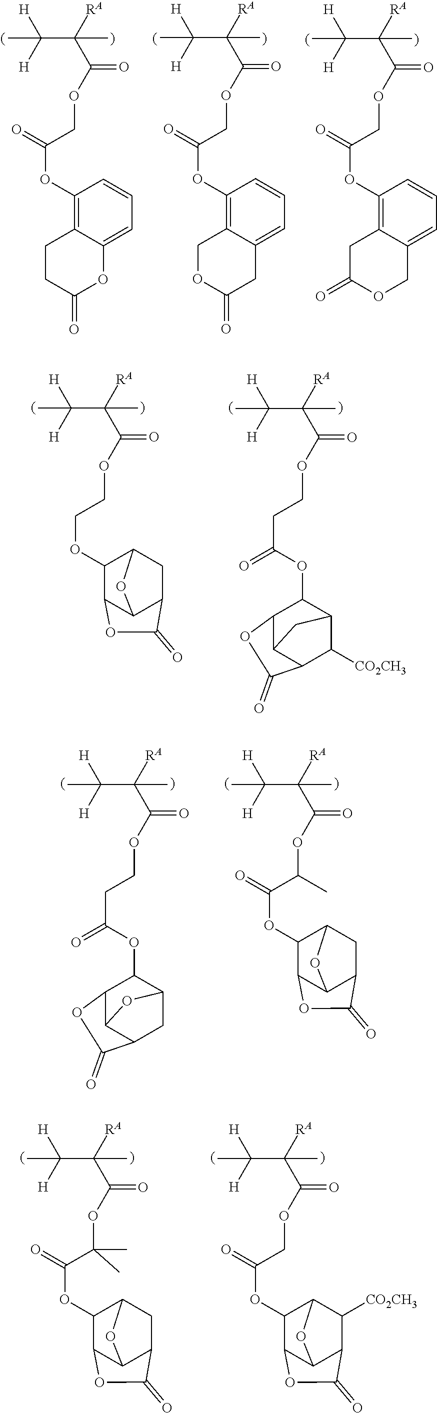

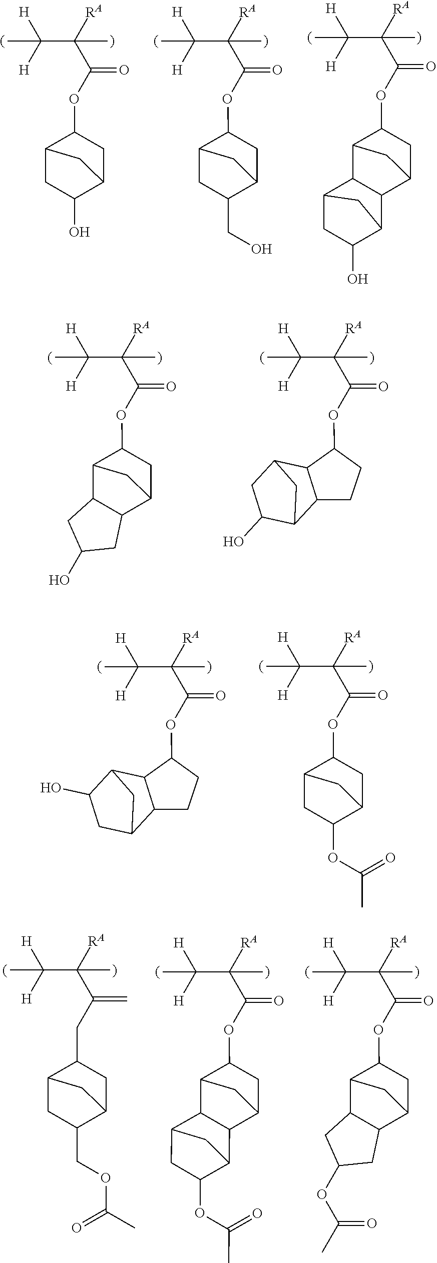

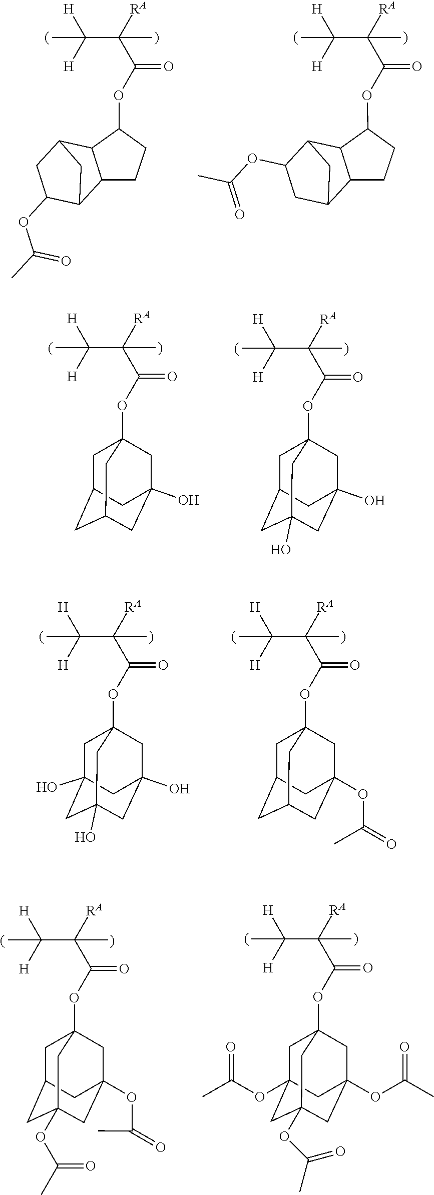

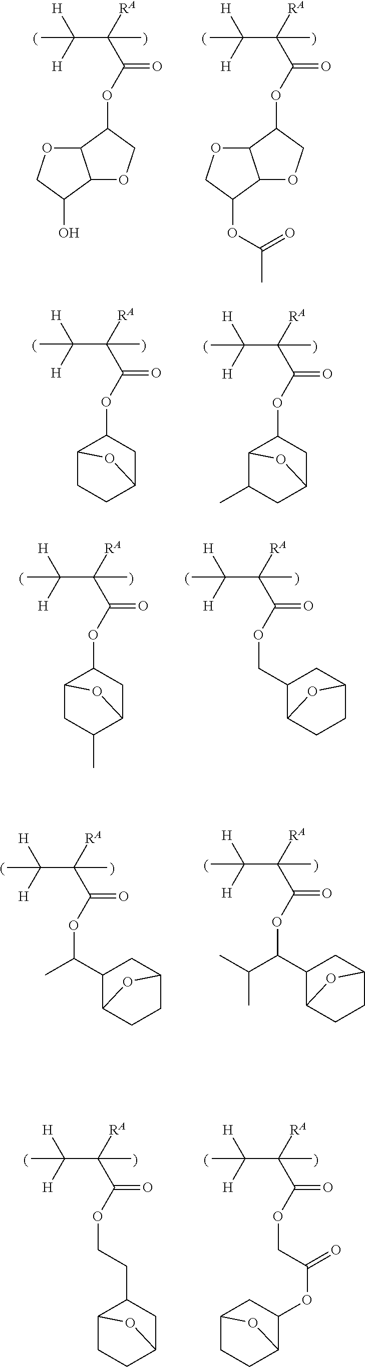

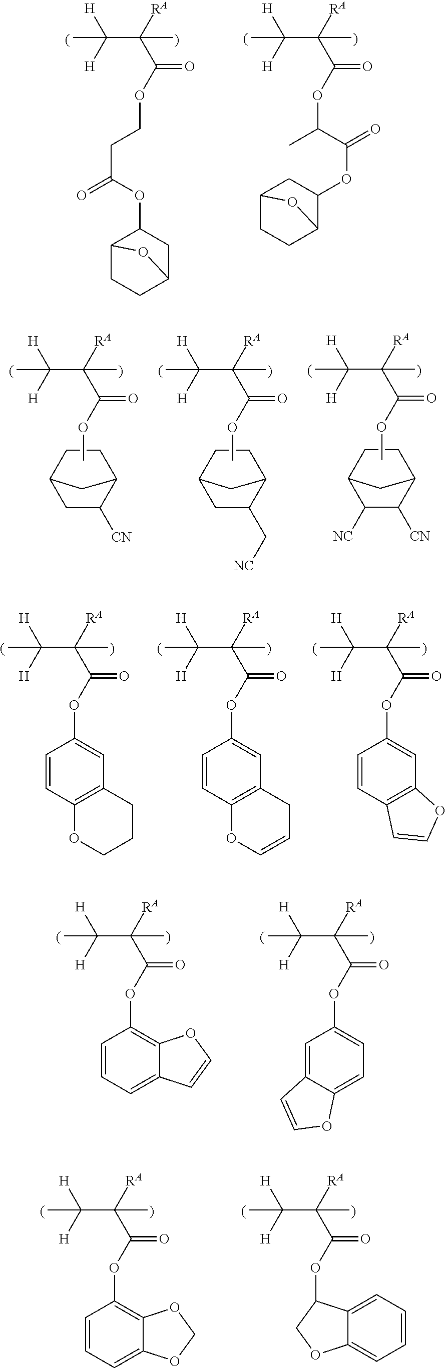

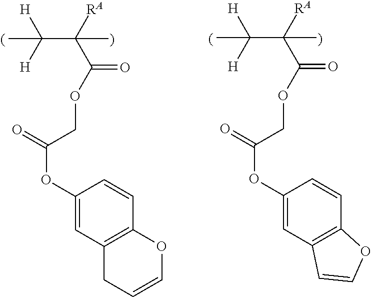

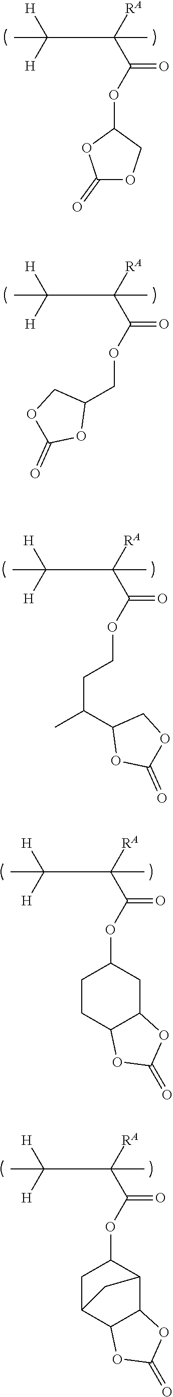

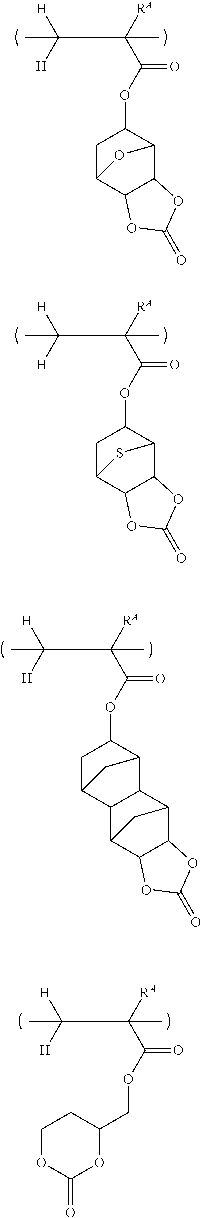

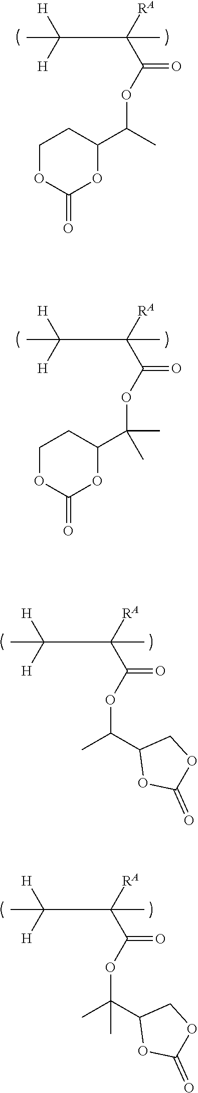

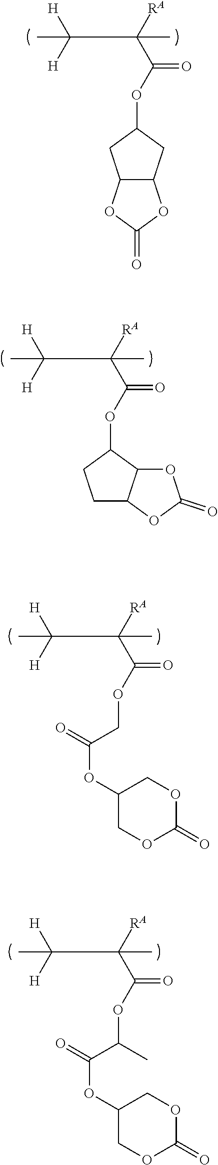

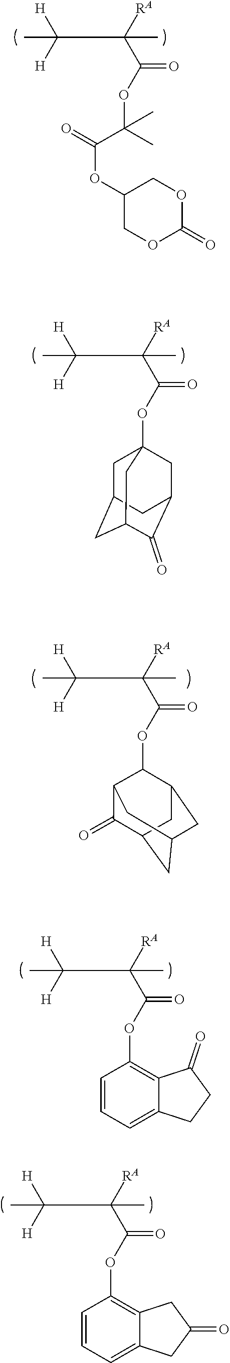

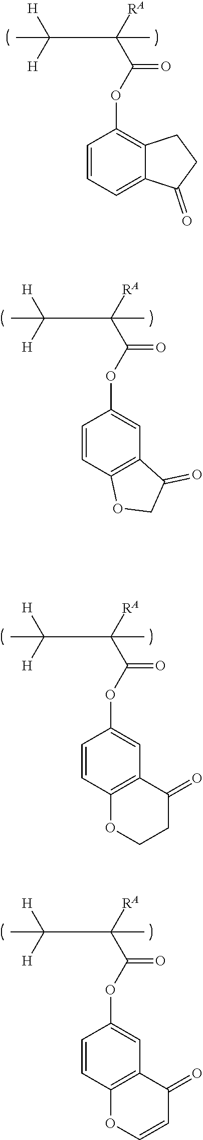

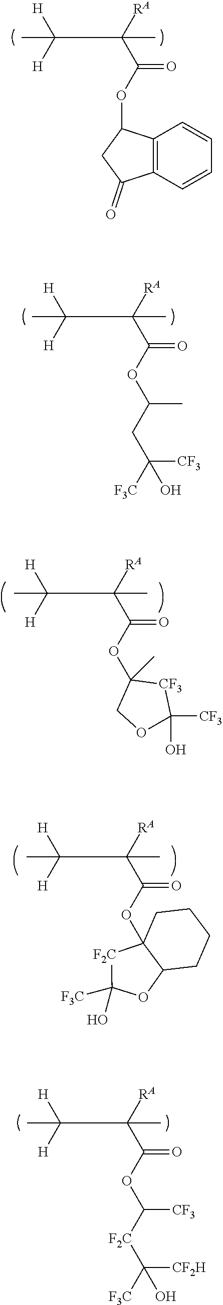

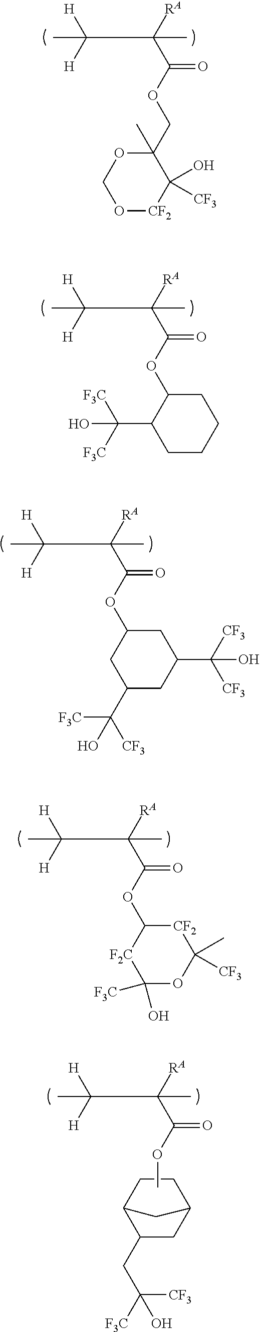

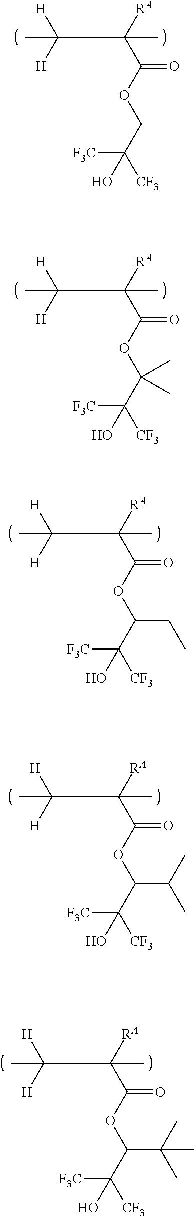

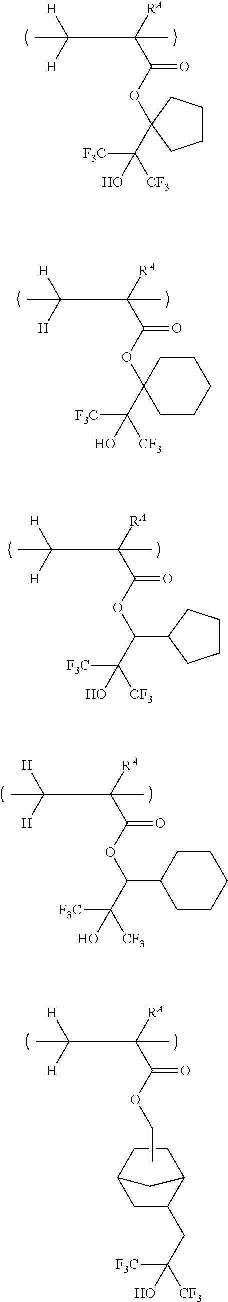

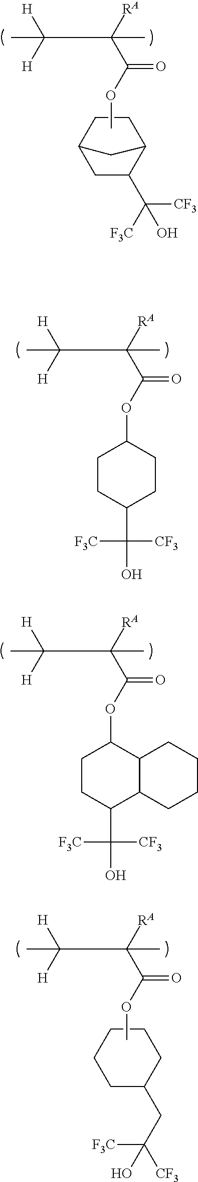

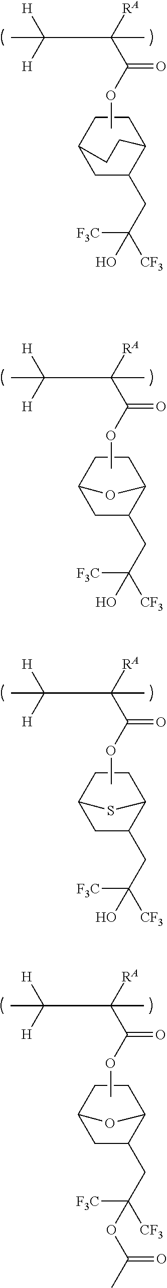

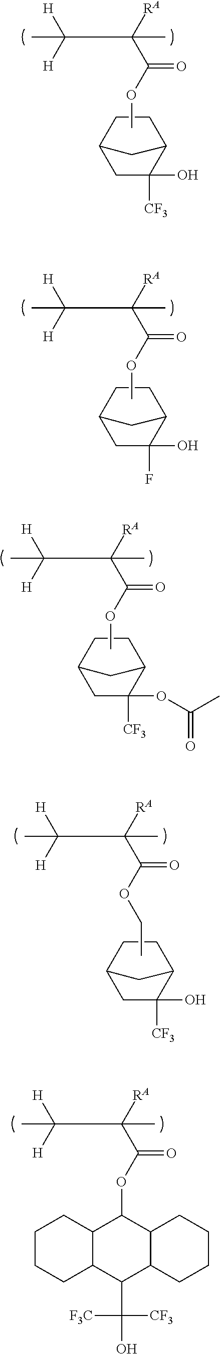

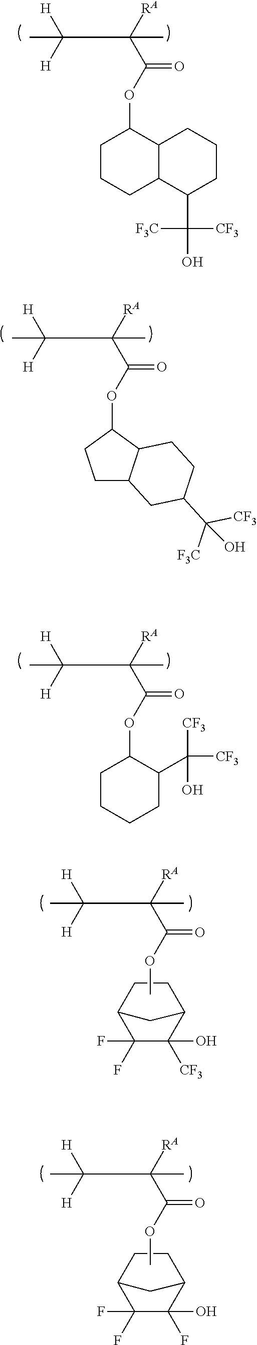

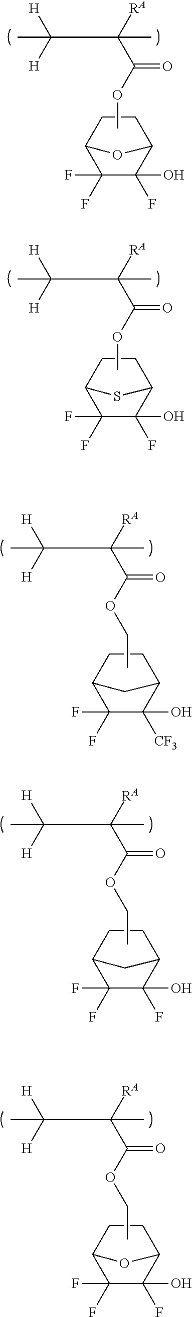

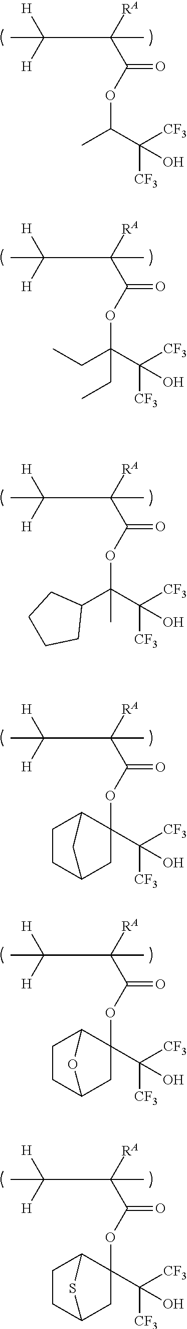

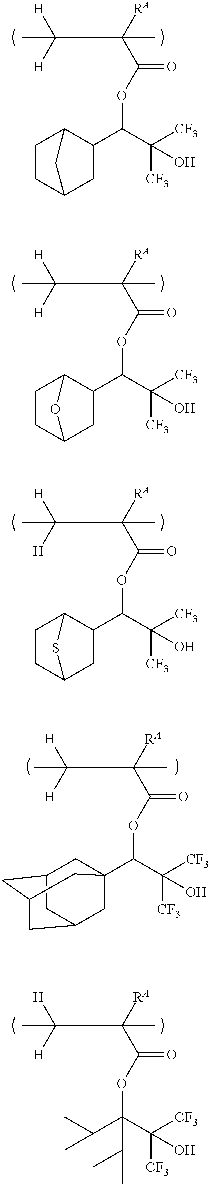

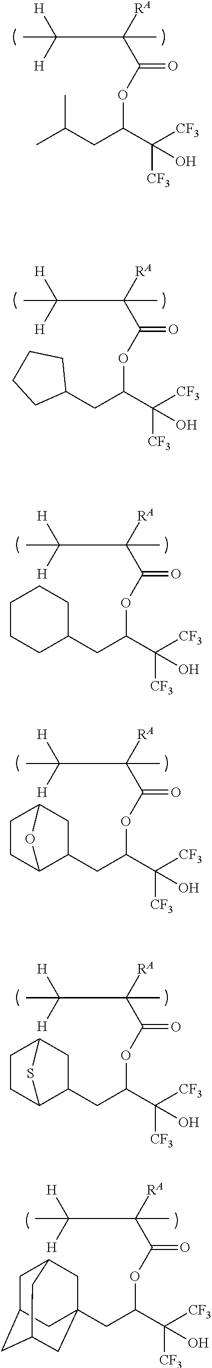

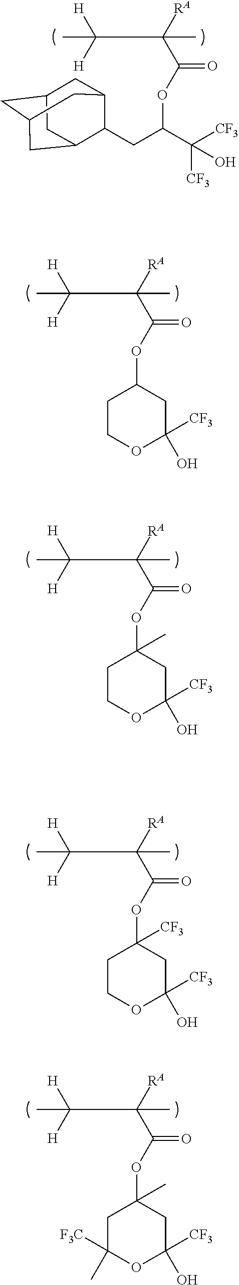

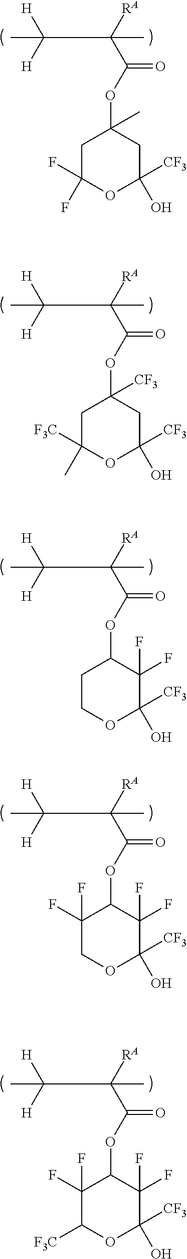

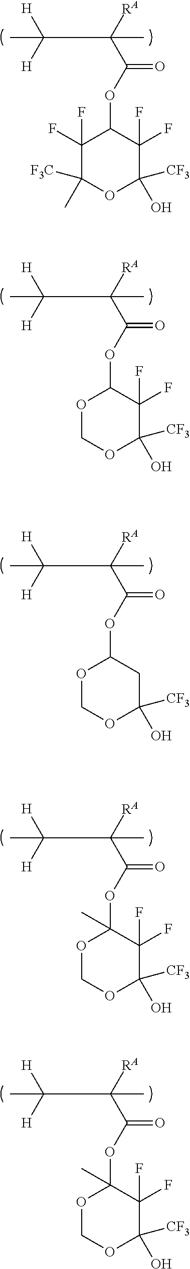

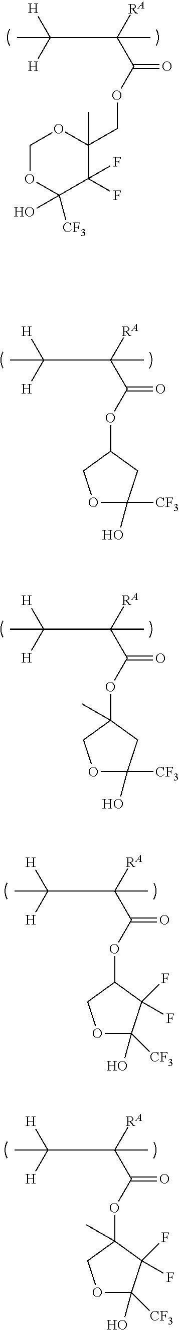

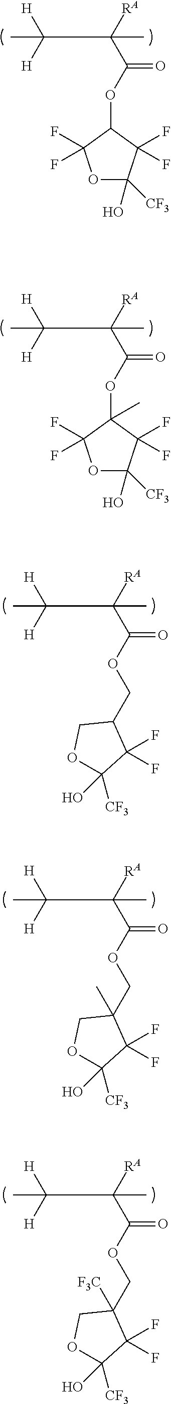

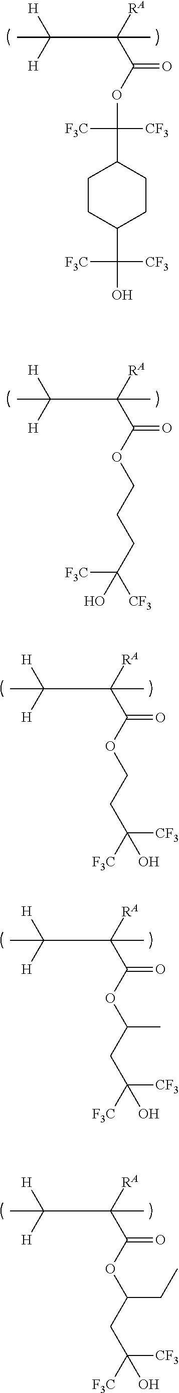

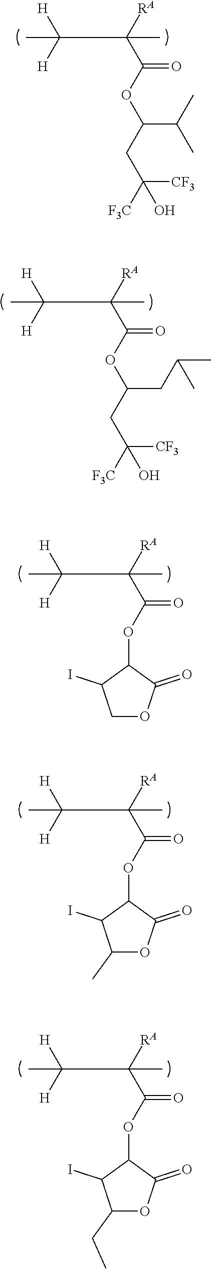

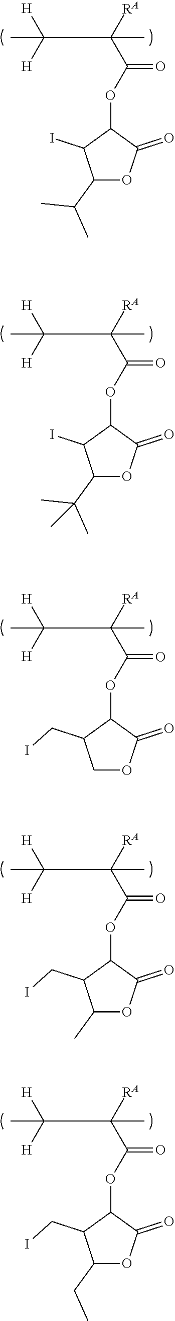

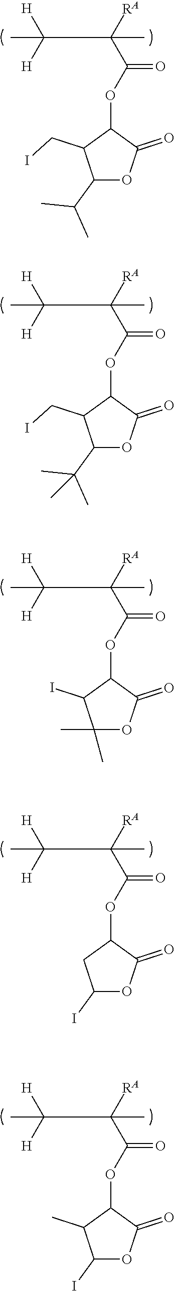

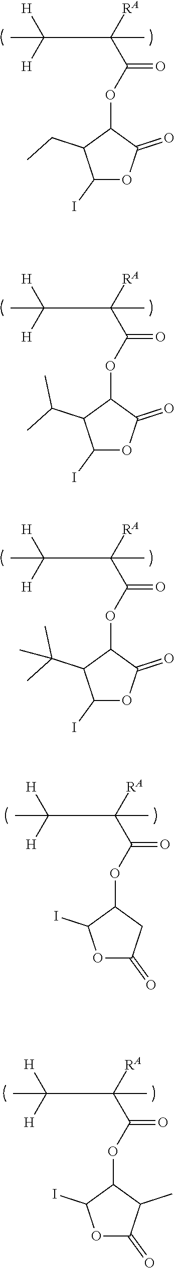

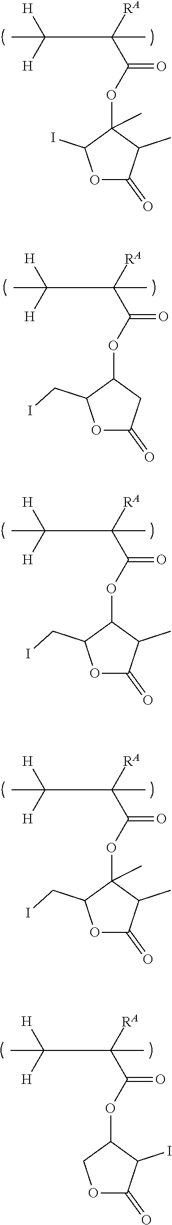

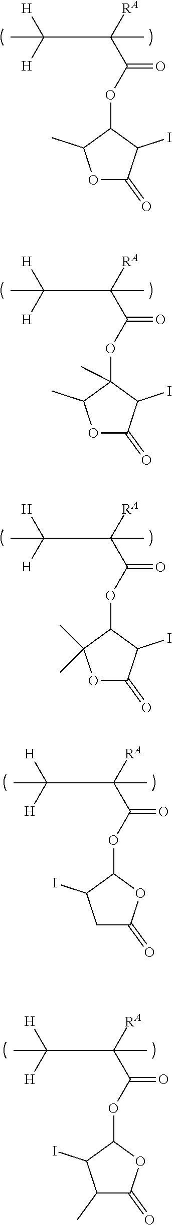

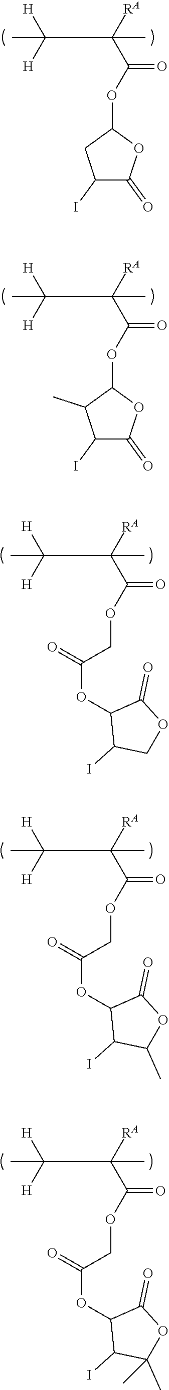

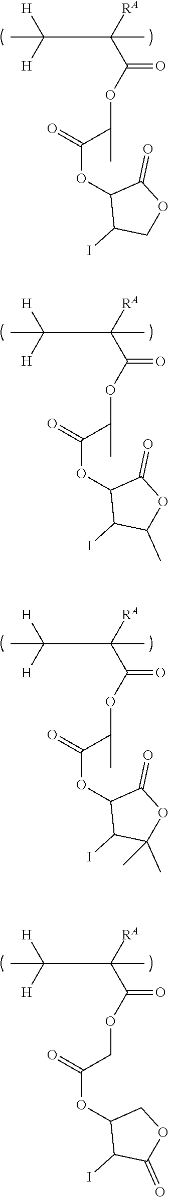

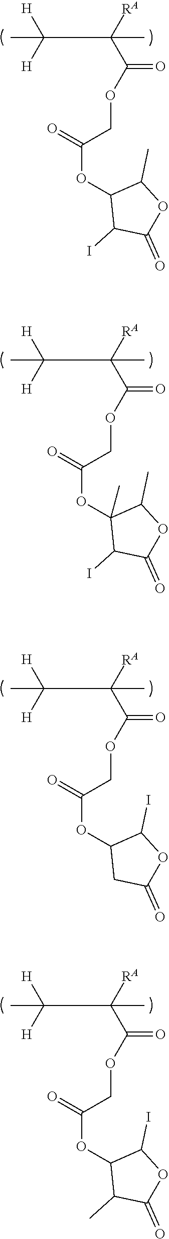

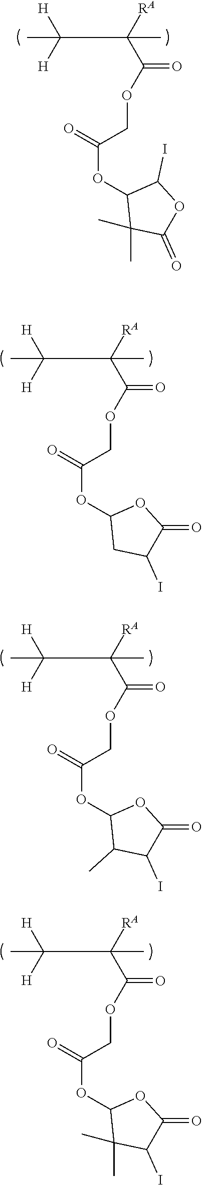

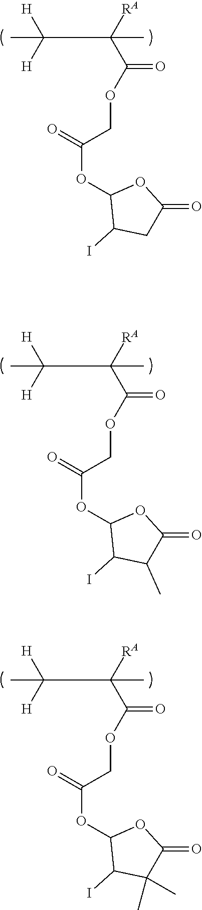

Specific examples of the repeat unit b2 are shown below, but not limited thereto. Herein R^{A} is as defined above.

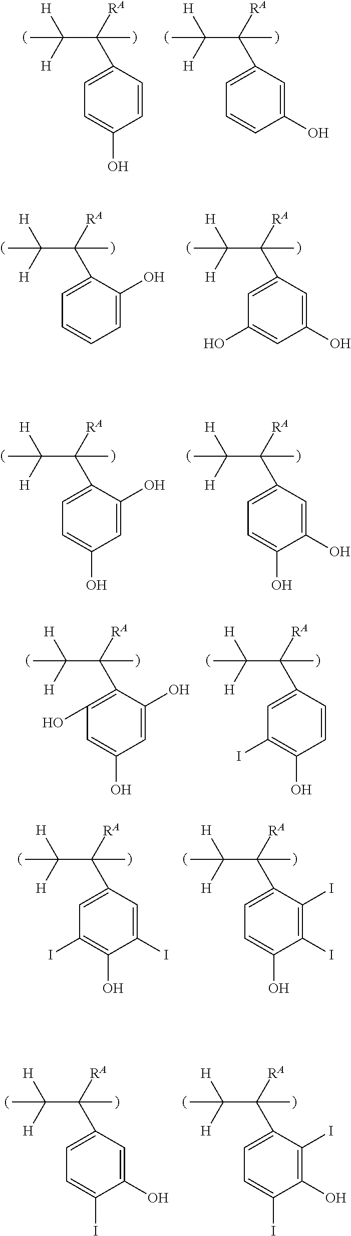

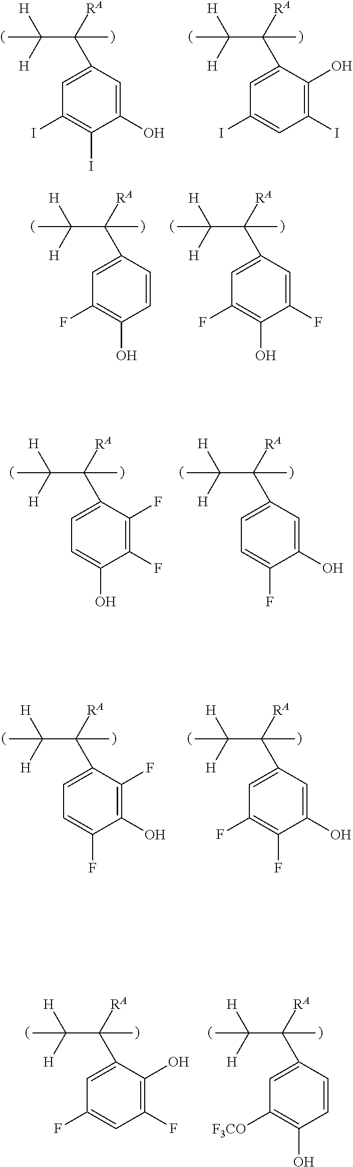

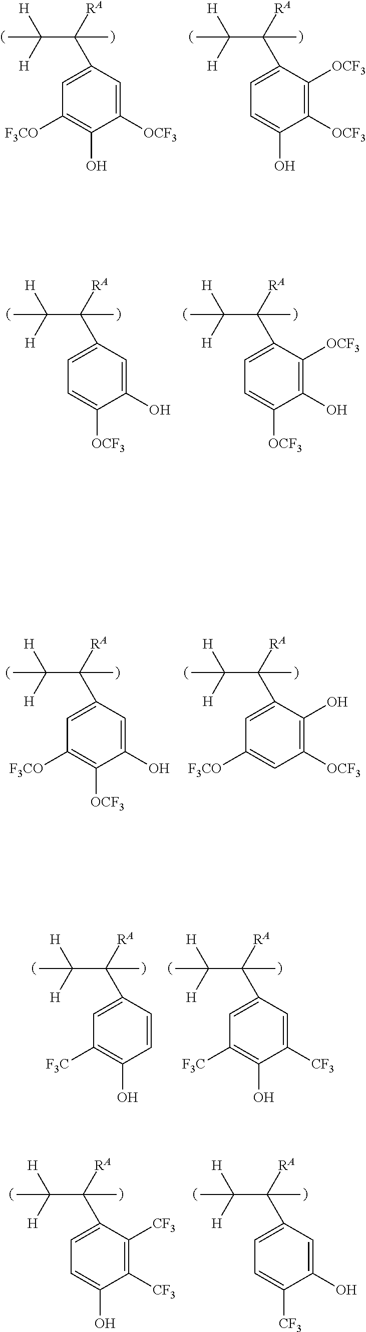

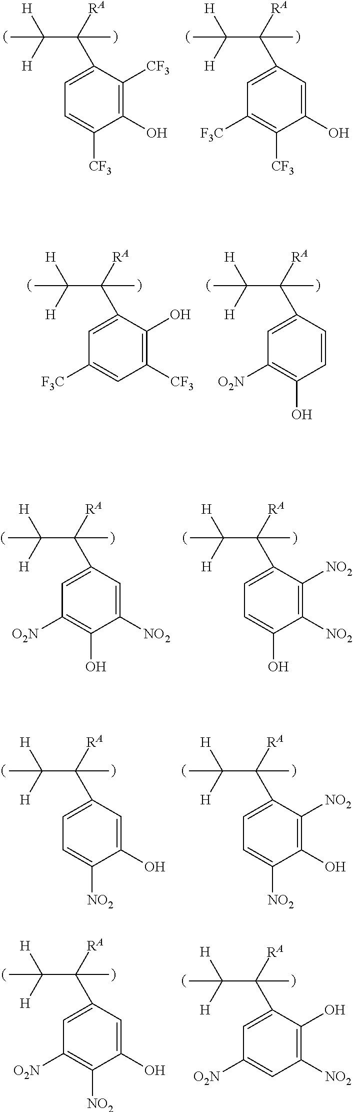

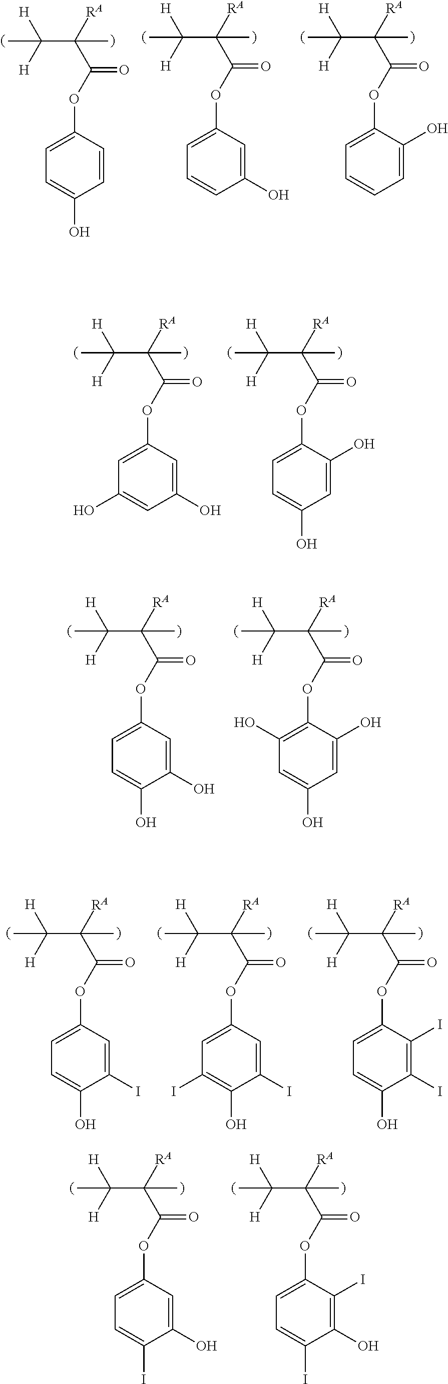

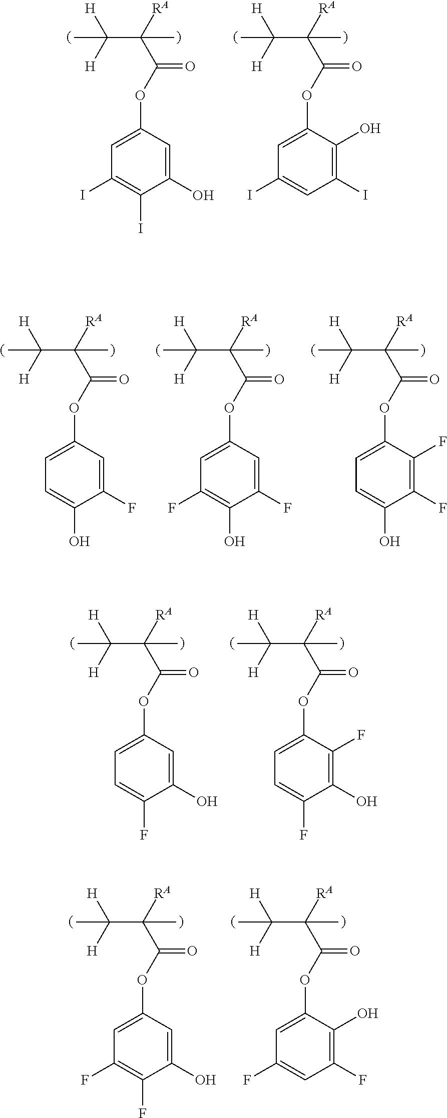

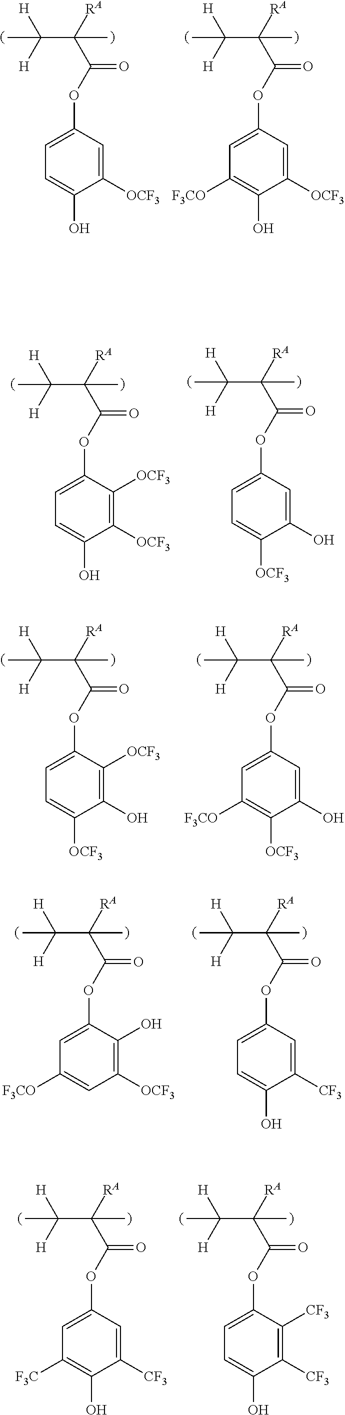

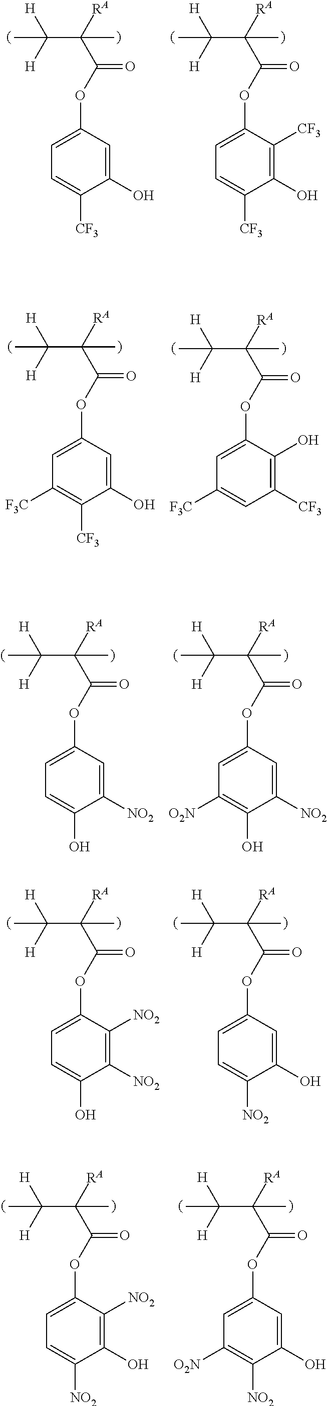

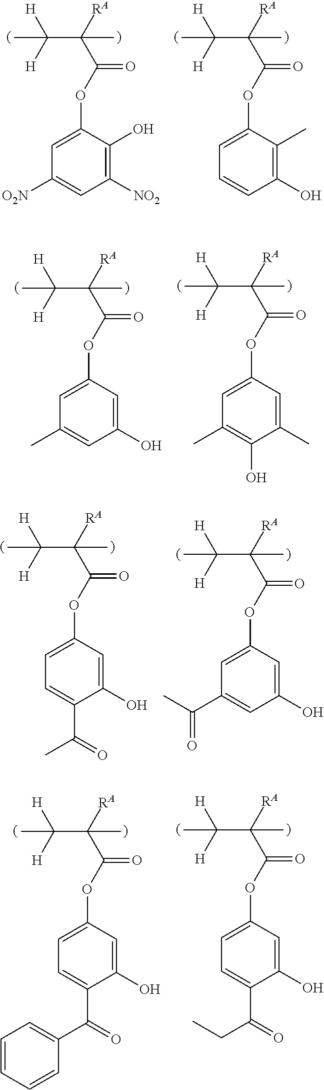

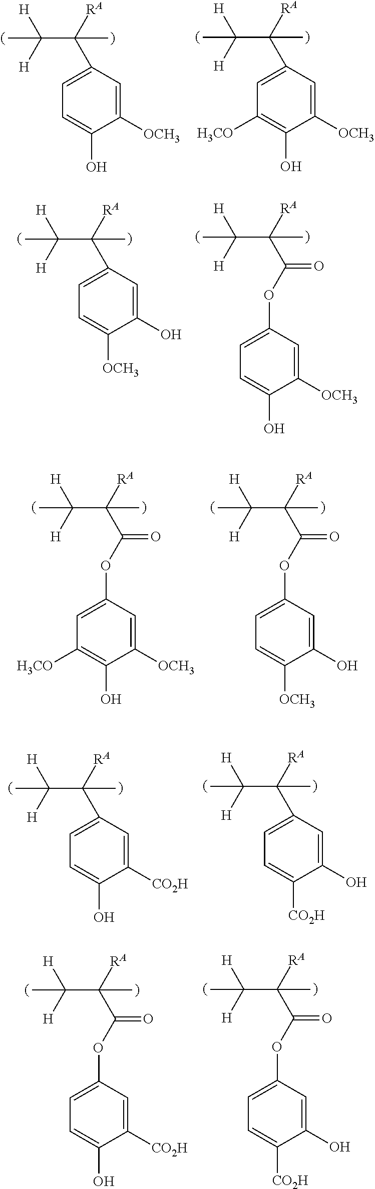

The repeat unit bl or b2 is particularly preferably one having a lactone ring as a polar group in ArF lithography, and is preferably one having a phenol site in KrF lithography, EB lithography, and EUV lithography.

The base polymer may further comprise at least one selected from a repeat unit having the formula (c1)(hereinafter, the repeat unit is also referred to as a repeat unit c1), a repeat unit having the formula (c2)(hereinafter, the repeat unit is also referred to as a repeat unit c2), a repeat unit having the formula (c3)(hereinafter, the repeat unit is also referred to as a repeat unit c3), a repeat unit having the formula (c4)(hereinafter, the repeat unit is also referred to as a repeat unit c4), and a repeat unit having the formula (c5) (hereinafter, the repeat unit is also referred to as a repeat unit c5).

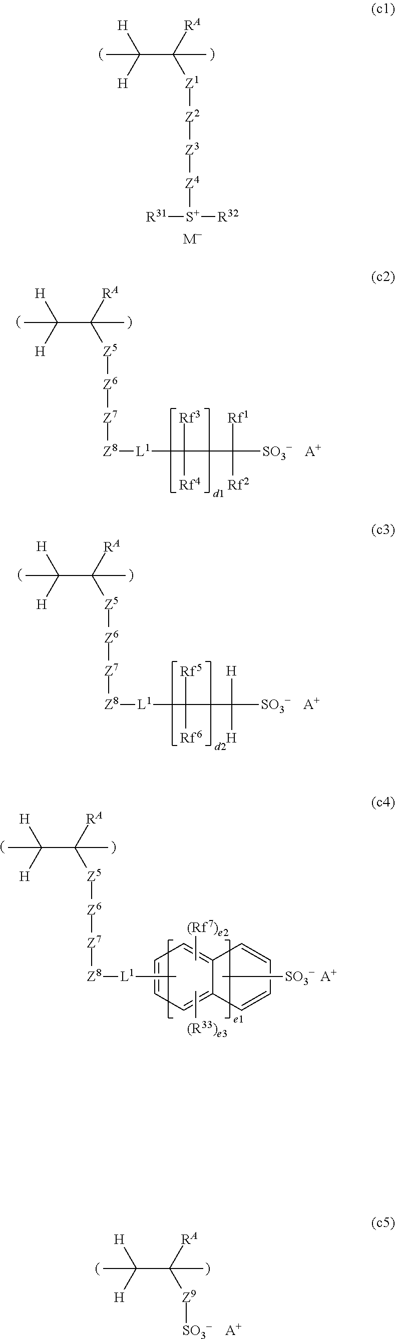

In formulae (c1) to (c5), R^{A} is each independently a hydrogen atom, a fluorine atom, a methyl group, or a trifluoromethyl group. Z¹ is a phenylene group which may have a single bond or a substituent. Z² is a single bond, **—C(═O)—O—Z²¹—, **—C(═O)—NH—Z²¹—, or **—O—Z²¹—. 721 is a C₁-C₆ aliphatic hydrocarbylene group, a phenylene group, or a divalent group obtained by combining the foregoing, and may contain a halogen atom, a carbonyl group, an ester bond, an ether bond, or a hydroxy group. Z³ is a single bond, an ether bond, an ester bond, an amide bond, a sulfonate ester bond, a sulfonamide bond, a carbonate bond, or a carbamate bond. Z⁴ is a single bond, a C₁-C₆ aliphatic hydrocarbylene group, a phenylene group, or a divalent group obtained by combining the foregoing, and may contain a halogen atom, a carbonyl group, an ester bond, an ether bond, or a hydroxy group. Z⁵ is each independently a single bond, a phenylene group which may have a substituent, a naphthylene group, or *—C(═O)—O—Z⁵¹—. Z⁵¹ is a C₁-C₁₀ aliphatic hydrocarbylene group, a phenylene group, or a naphthylene group, and the aliphatic hydrocarbylene group may contain a halogen atom, a hydroxy group, an ether bond, an ester bond, or a lactone ring. Z⁶ is a single bond, an ether bond, an ester bond, an amide bond, a sulfonate ester bond, a sulfonamide bond, a carbonate bond, or a carbamate bond. Z⁷ is each independently a single bond, ***—Z⁷¹—C(═O)—O—, ***—C(═O)—NH—Z⁷¹—, or ***—O—Z⁷¹—. Z⁷¹ is a C₁-C₂₀ hydrocarbylene group which may contain a heteroatom. Z⁸ is each independently a single bond, ****—Z⁸¹—C(—O)—O—, ****—C(═O)—NH—Z⁸¹—, or —O—Z⁸¹—. Z⁸¹ is a C₁-C₂₀ hydrocarbylene group which may contain a heteroatom. Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—N(H)—Z⁹¹—, or *—O—Z⁹¹—. Z⁹¹ is a phenylene group substituted with a C₁-C₆ aliphatic hydrocarbylene group, a phenylene group, a fluorinated phenylene group, or a trifluoromethyl group, and may contain a carbonyl group, an ester bond, an ether bond, or a hydroxy group. * designates a point of attachment to the carbon atom in the backbone. * designates a point of attachment to Z¹. *** designates a point of attachment to Z⁶. **** designates a point of attachment to Z⁷.

The aliphatic hydrocarbylene groups Z^{21, 751}, and Z⁹¹ may be straight, branched, or cyclic, and specific examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl, and cyclohexanediyl, and combinations thereof.

The hydrocarbylene groups Z⁷¹ and Z⁸¹ which may contain a heteroatom may be saturated or unsaturated and straight, branched, or cyclic. Specific examples thereof are shown below, but not limited thereto.

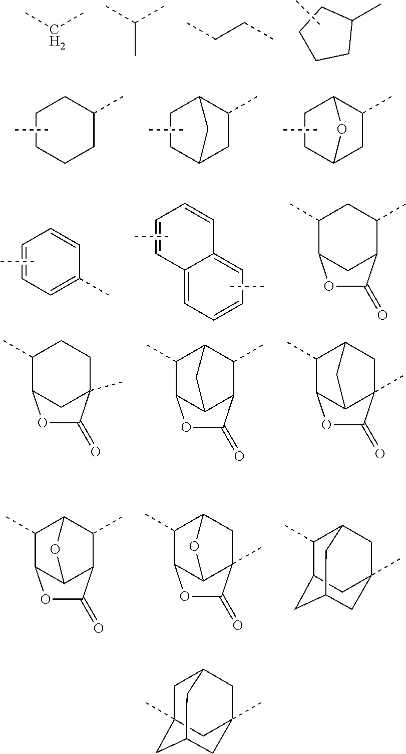

Herein the broken line designates a point of attachment.

In formula (c1), R³¹ and R³² are each independently a C₁-C₂₀ hydrocarbyl group which may contain a heteroatom. The hydrocarbyl group may be saturated or unsaturated and straight, branched, or cyclic. Specific examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl, naphthyl, and thienyl; C₇-C₂₀ aralkyl groups such as benzyl, 1-phenylethyl, and 2-phenylethyl, and combinations thereof, and the aryl groups are preferred. In the hydrocarbyl group, some or all of the hydrogen atoms may be substituted by a moiety containing a heteroatom such as an oxygen atom, a sulfur atom, a nitrogen atom, or a halogen atom, and some constituent —CH₂—may be replaced by a moiety containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

R³¹ and R³² may bond together to form a ring with the sulfur atom to which they are attached. Specific examples of the ring are shown below.

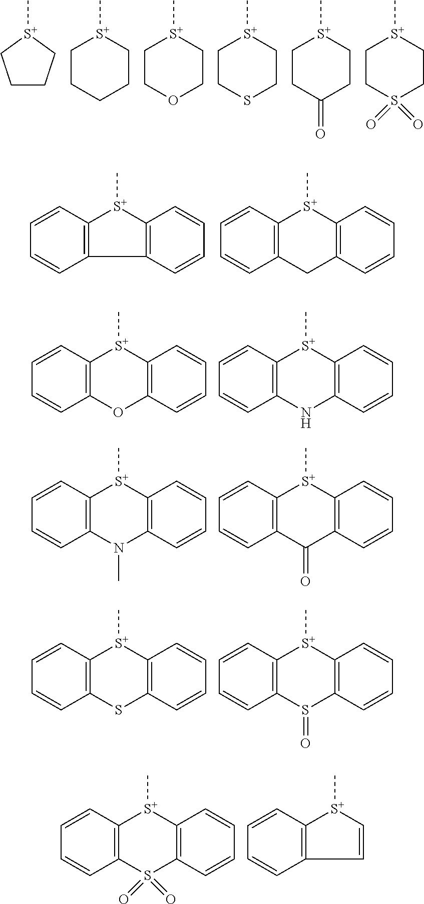

Herein the broken line designates a point of attachment to Z⁴.

Specific examples of the cation of the repeat unit c1 are shown below, but not limited thereto. Herein R^{A} is as defined above.

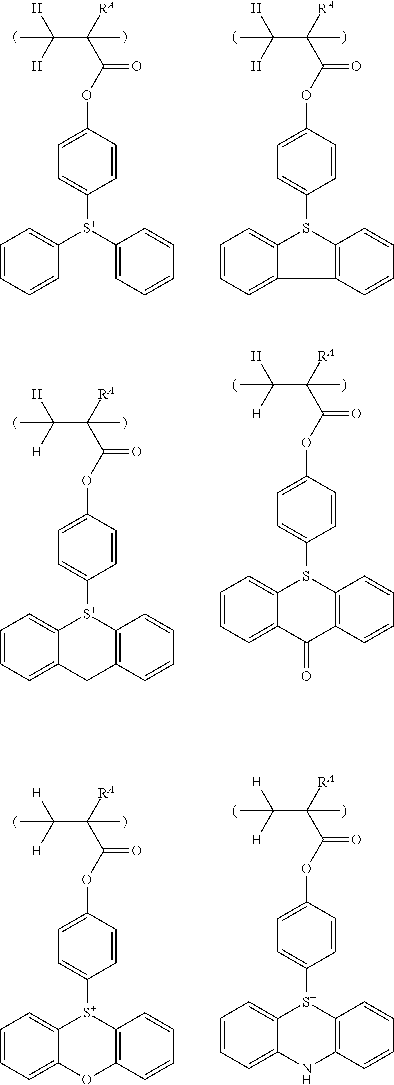

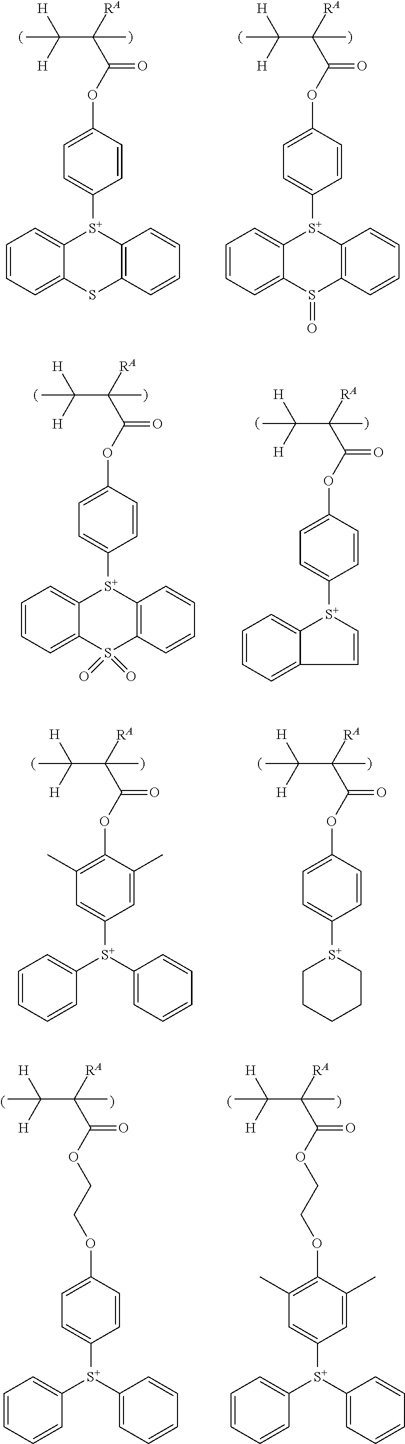

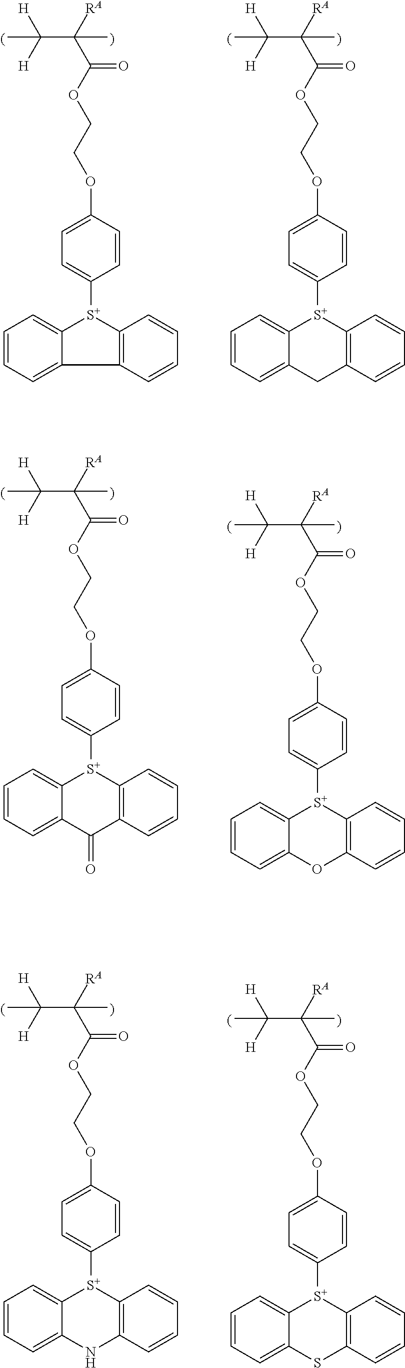

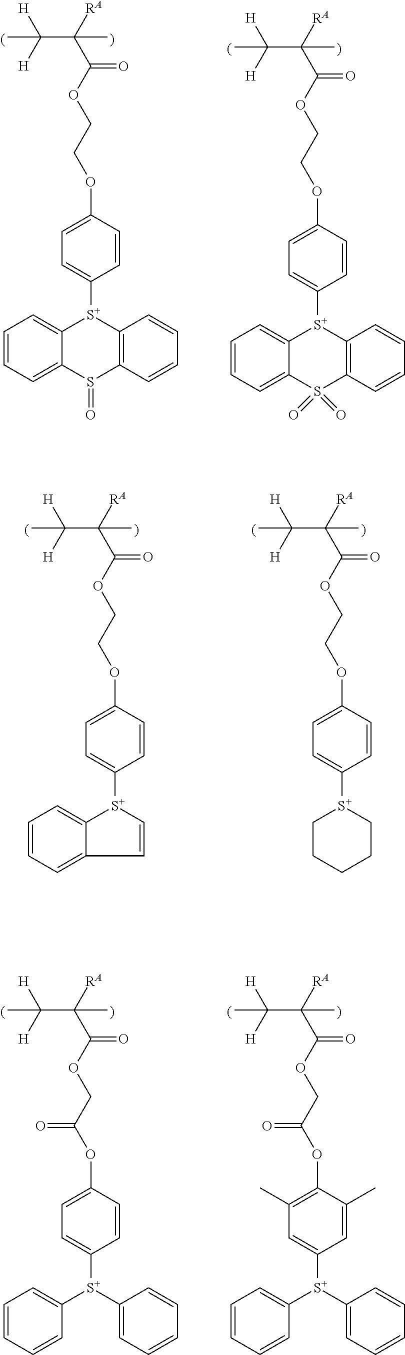

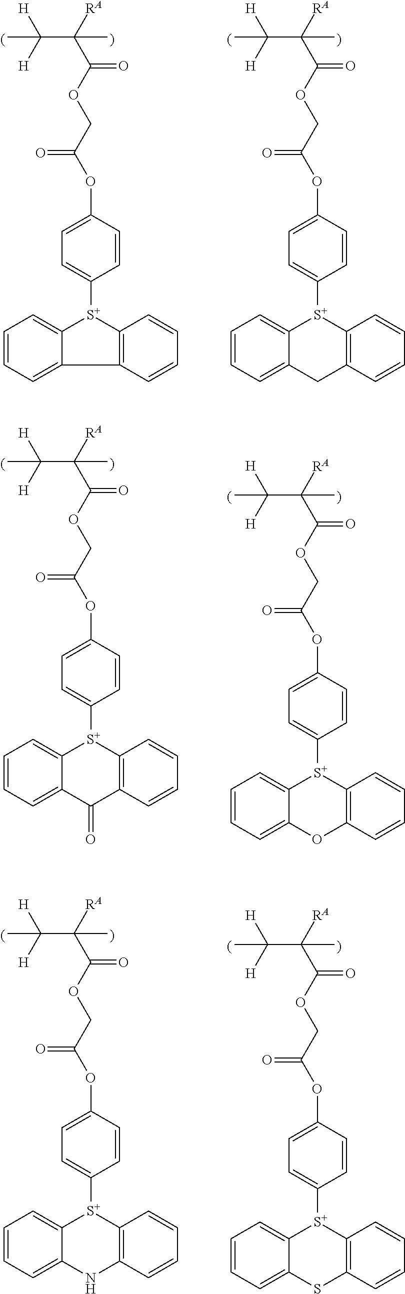

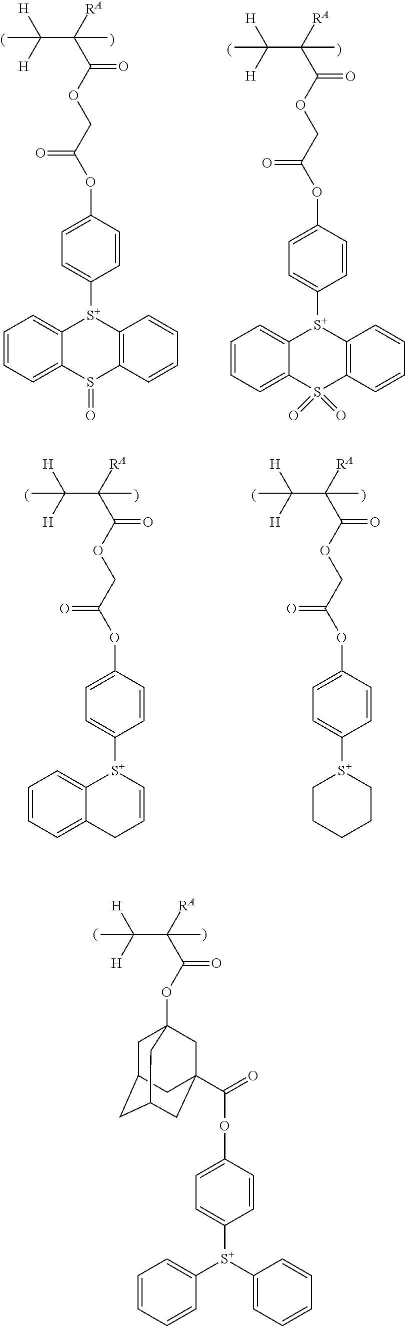

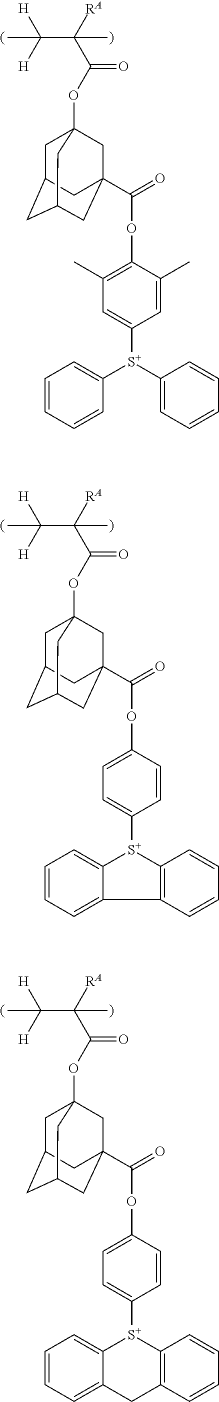

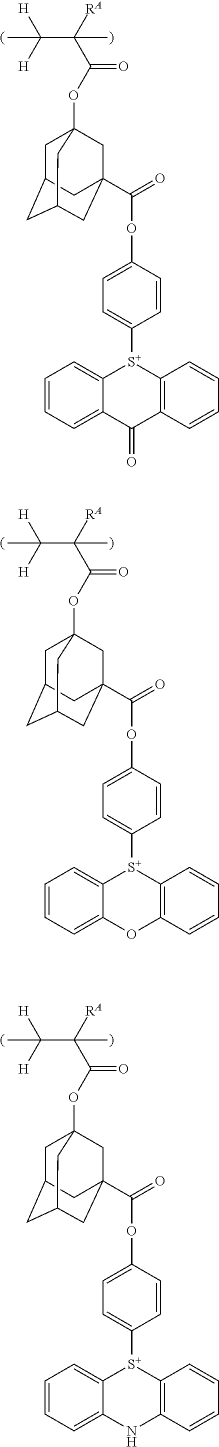

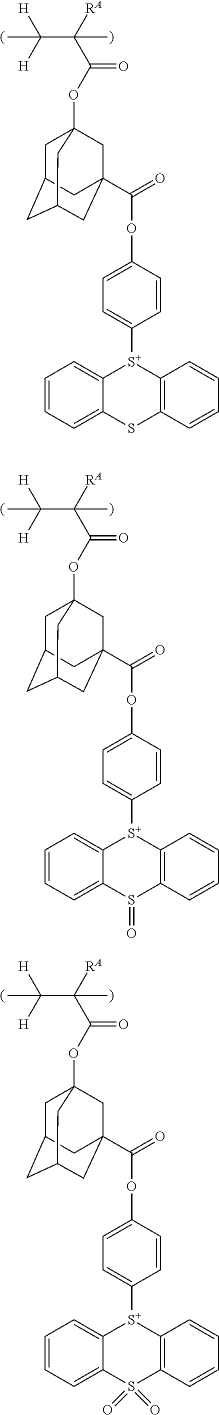

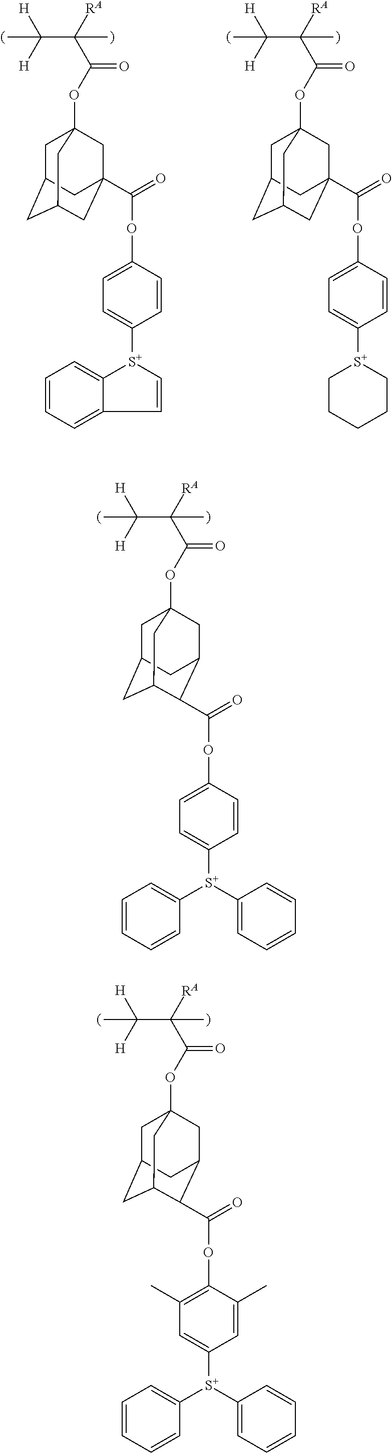

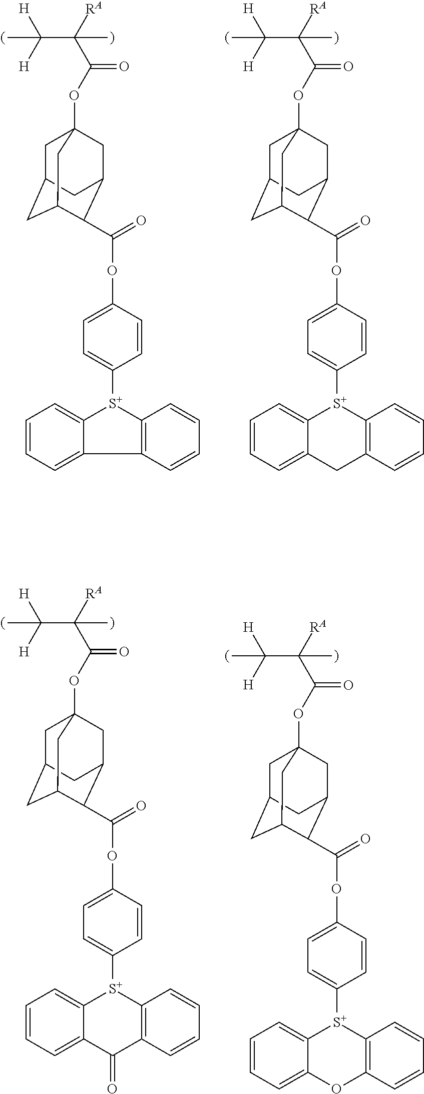

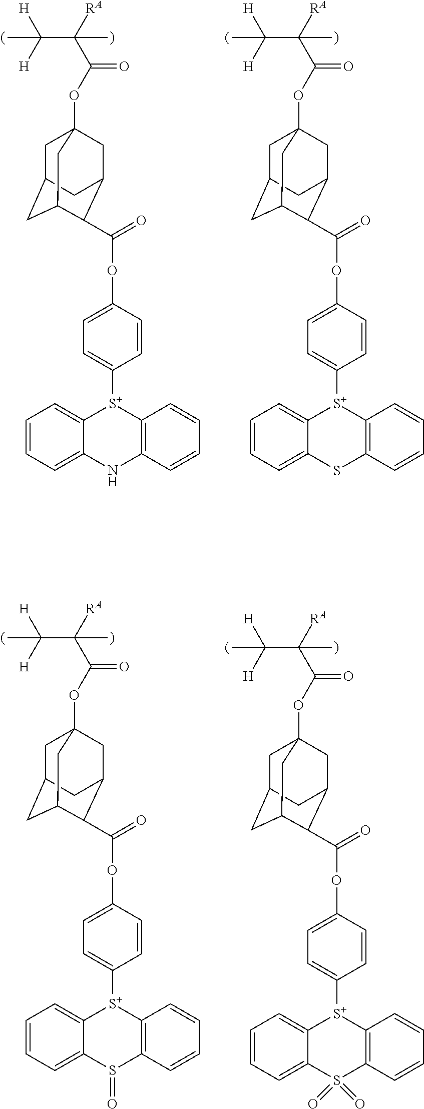

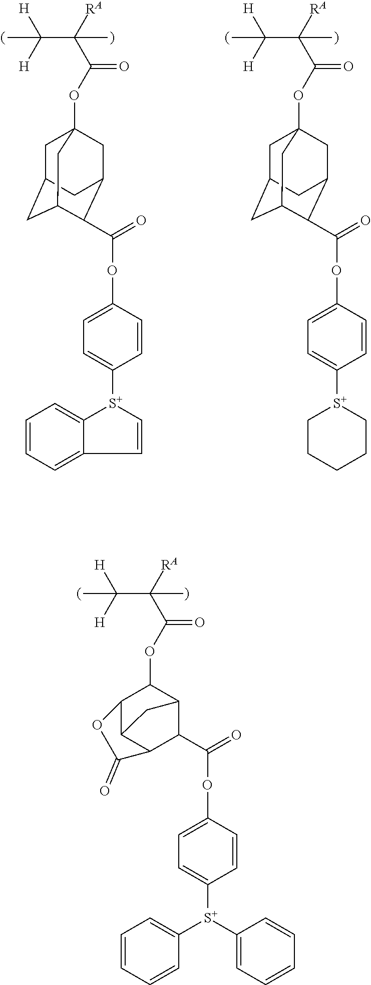

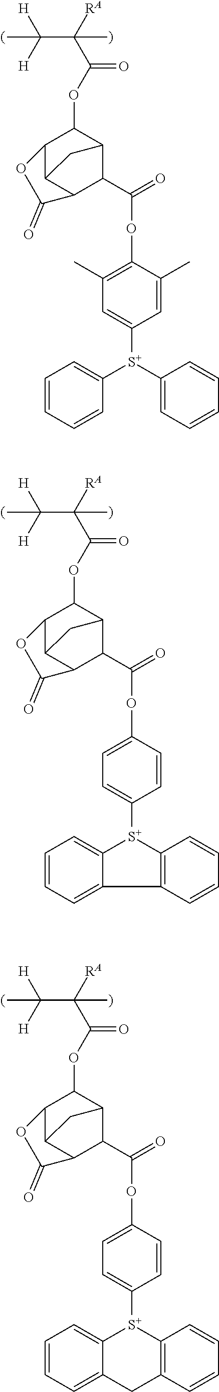

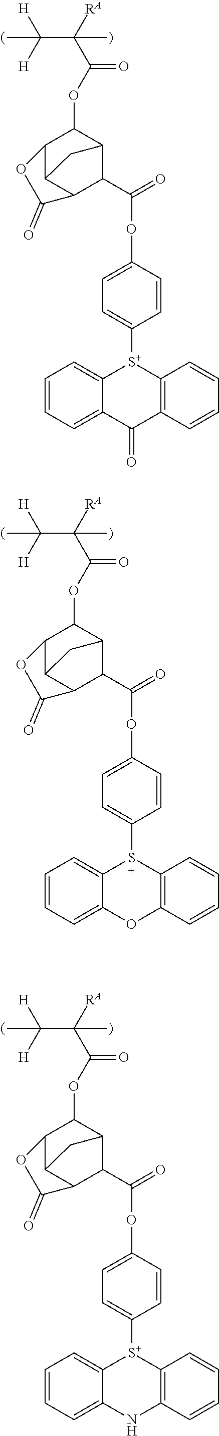

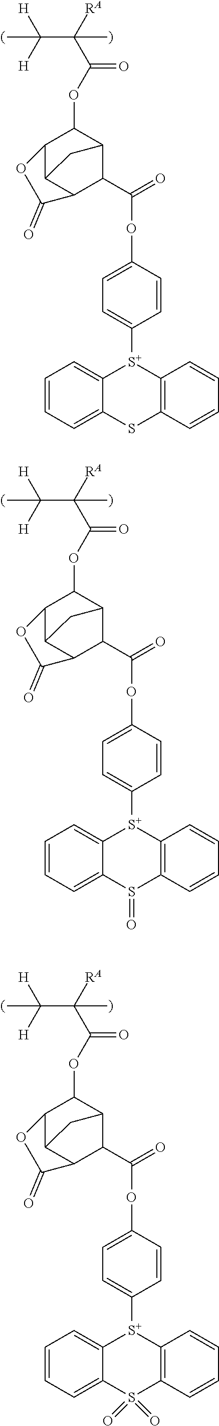

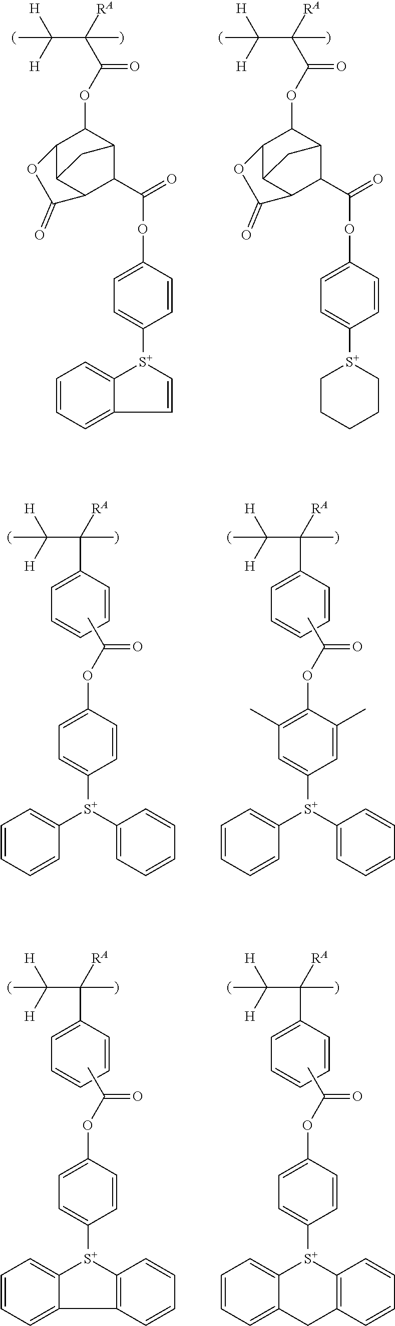

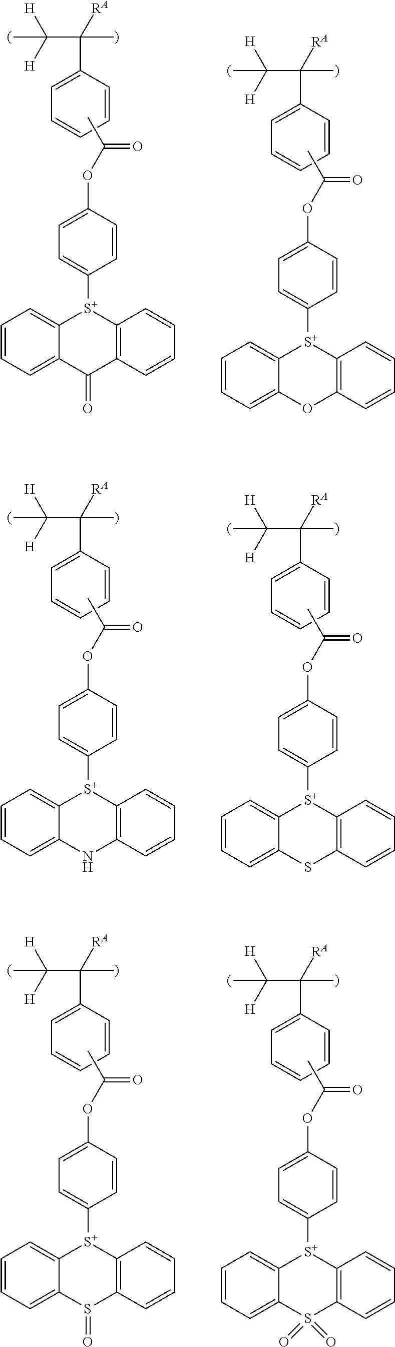

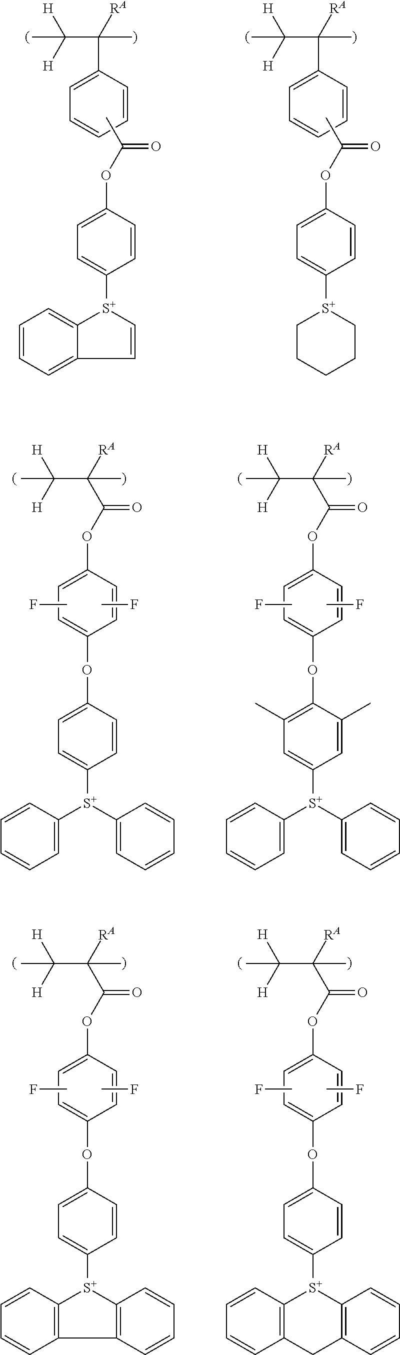

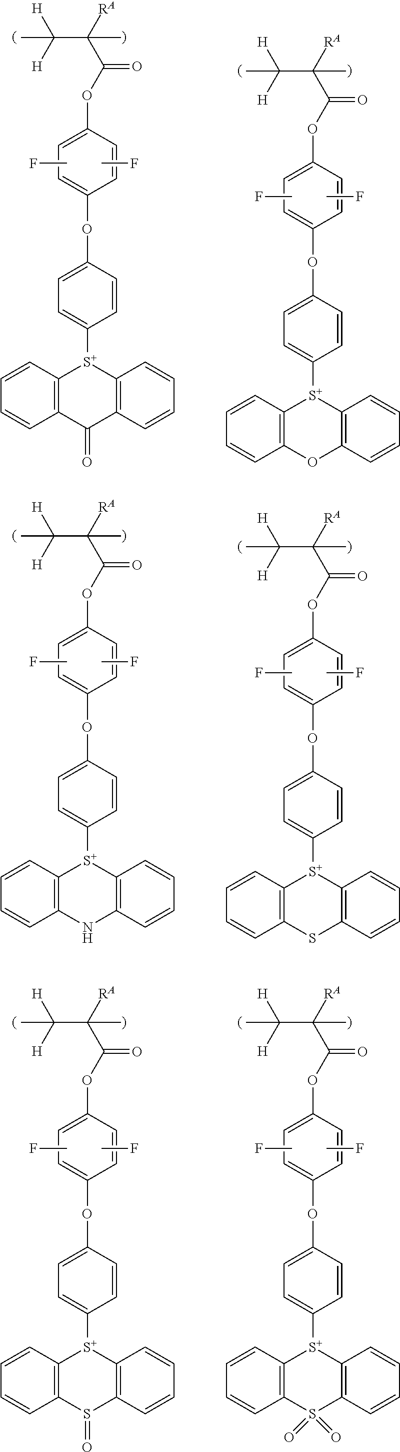

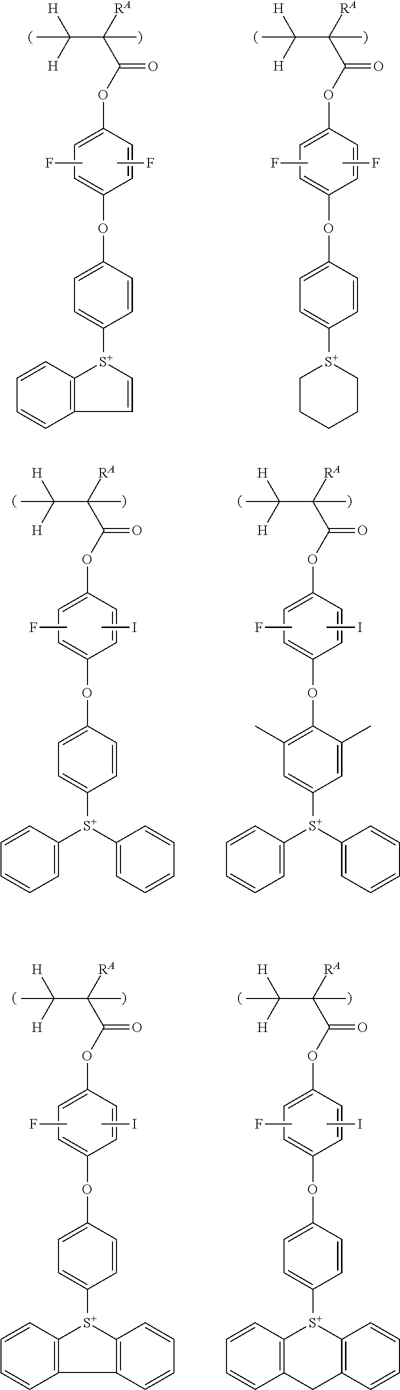

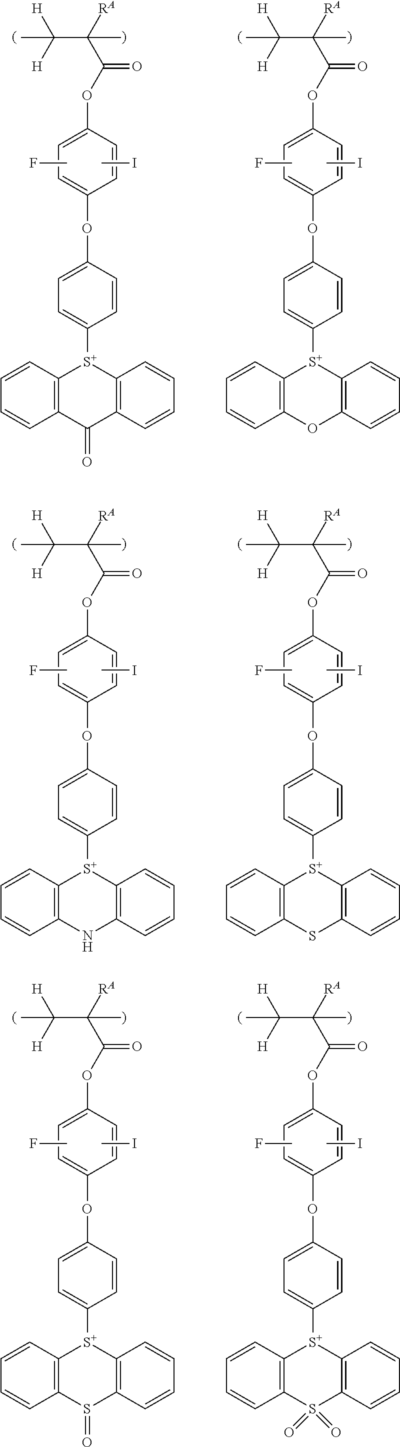

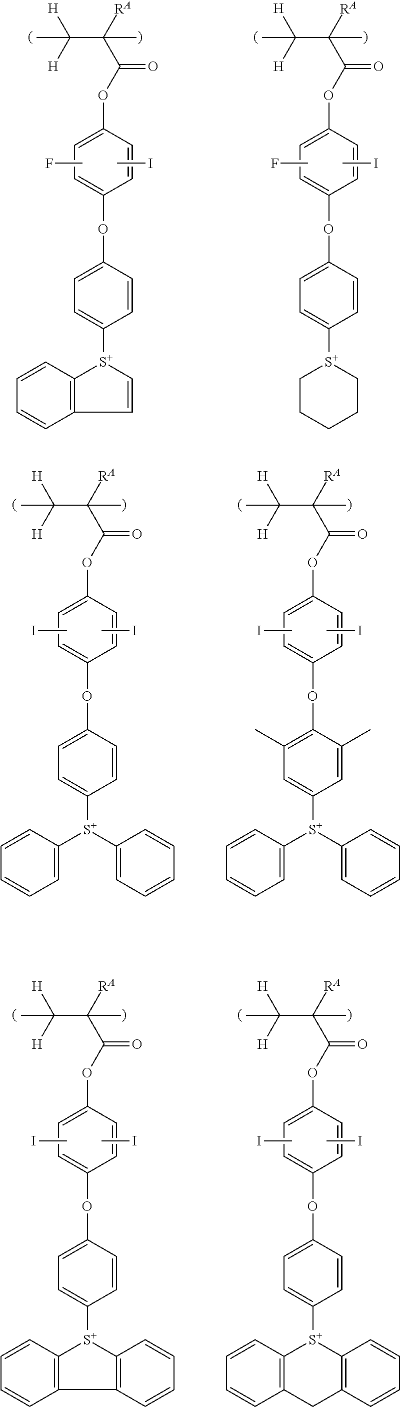

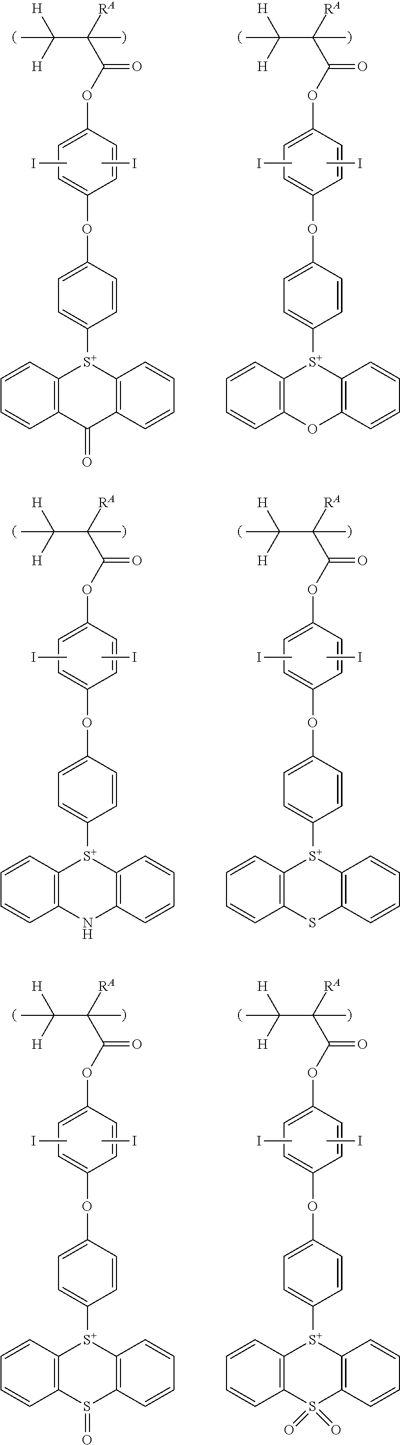

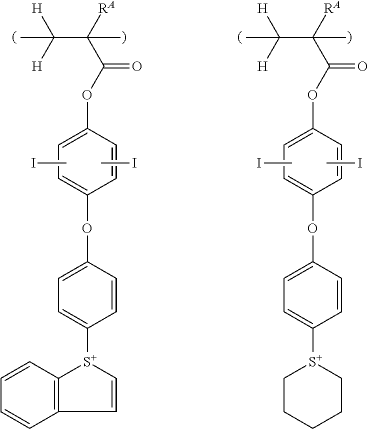

In formula (c1), M⁻ is a non-nucleophilic counter ion. The non-nucleophilic counter ion is preferably a halide ion, a sulfonate anion, an imide anion, or a methide anion. Specific examples of the halide ion include a chloride ion and a bromide ion. Specific examples of the sulfonate anion include fluoroalkylsulfonate ions such as triflate, 1,1,1-trifluoroethanesulfonate, and nonafluorobutanesulfonate, arylsulfonate ions such as tosylate, benzenesulfonate, 4-fluorobenzenesulfonate, and 1,2,3,4,5-pentafluorobenzenesulfonate, and alkylsulfonate ions such as mesylate and butanesulfonate. Specific examples of the imide ions include bis(trifluoromethylsulfonyl)imide, bis(perfluoroethylsulfonyl)imide, and bis(perfluorobutylsulfonyl)imide. Specific examples of the methide ions include tris(trifluoromethylsulfonyl) methide and tris(perfluoroethylsulfonyl) methide.

Other examples of the non-nucleophilic counter ion include anions having the formulae (c1-1) to (c1-4).

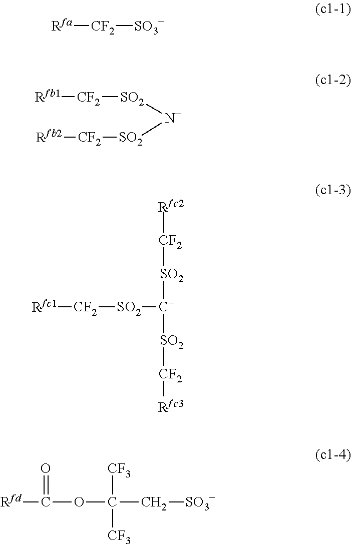

In formula (c1-1), R^{fa} is a fluorine atom or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group R^{fa1} in formula (c1-1-1).

As the anion having formula (c1-1), an anion having the formula (c1-1-1) is preferred.

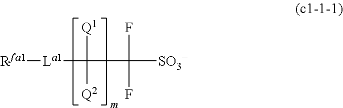

In formula (c1-1-1), Q¹ and Q² are each independently a hydrogen atom, a fluorine atom, or a C₁-C₆ fluorinated saturated hydrocarbyl group, and at least one of Q¹ and Q² is preferably a trifluoromethyl group for improving solvent solubility. m is 0, 1, 2, 3, or 4, and is particularly preferably 1. R^{fa1} is a C₁-C₃₅ hydrocarbyl group which may contain a heteroatom. The heteroatom is preferably an oxygen atom, a nitrogen atom, a sulfur atom, a halogen atom, or the like, more preferably an oxygen atom. As the hydrocarbyl group, a C₆-C₃₀ hydrocarbyl group is particularly preferred from the viewpoint of obtaining high resolution in small-size pattern formation.

In formula (c1-1-1), the C₁-C₃₅ hydrocarbyl group R^{fa1} may be saturated or unsaturated and straight, branched, or cyclic. Specific examples thereof include C₁-C₃₅ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl; C₃-C₃₅ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₃₅ unsaturated aliphatic hydrocarbyl groups such as 2-propenyl group and 3-cyclohexenyl; C₆-C₃₅ aryl groups such as phenyl, 1-naphthyl, 2-naphthyl and 9-fluorenyl; C₇-C₃₅ aralkyl groups such as benzyl and diphenylmethyl; and combinations thereof.

In the hydrocarbyl group, some or all of the hydrogen atoms may be substituted by a moiety containing a heteroatom such as an oxygen atom, a sulfur atom, a nitrogen atom, or a halogen atom, and some constituent —CH₂—may be replaced by a moiety containing a heteroatom such as an oxygen atom, a sulfur atom, or a nitrogen atom, so that the group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—) or a haloalkyl group. Specific examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

In formula (c1-1-1), Lal is a single bond, an ether bond, an ester bond, a sulfonate ester bond, a carbonate bond, or a carbamate bond, and from the viewpoint of synthesis, L^{a1} is preferably an ether bond or an ester bond, more preferably an ester bond.

Examples of the anion having formula (c1-1) are shown below, but not limited thereto. Herein Q¹ is as defined above, and Ac is an acetyl group.

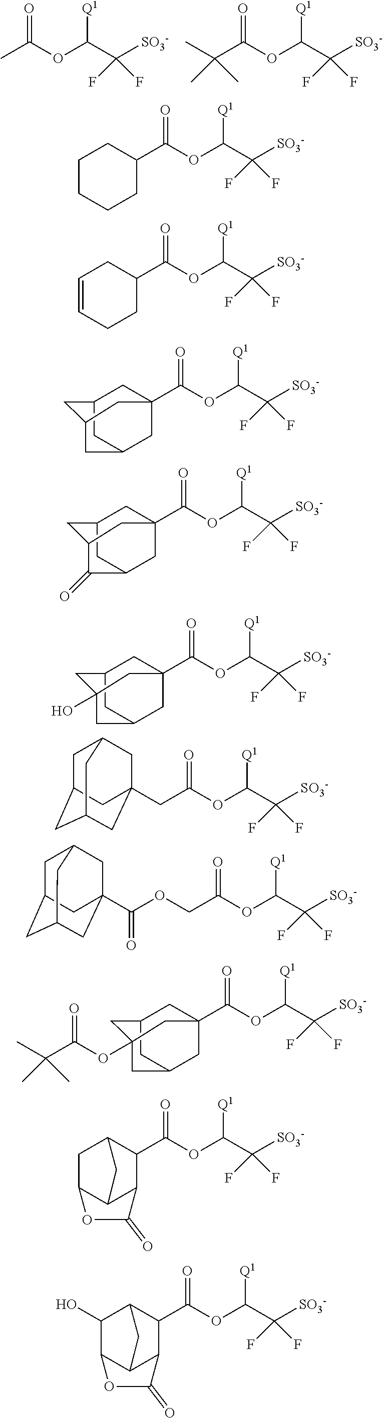

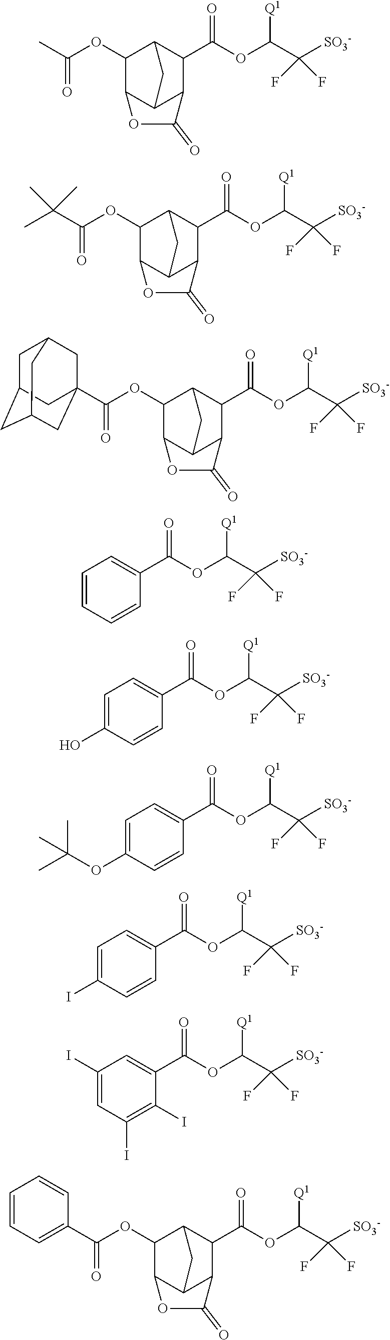

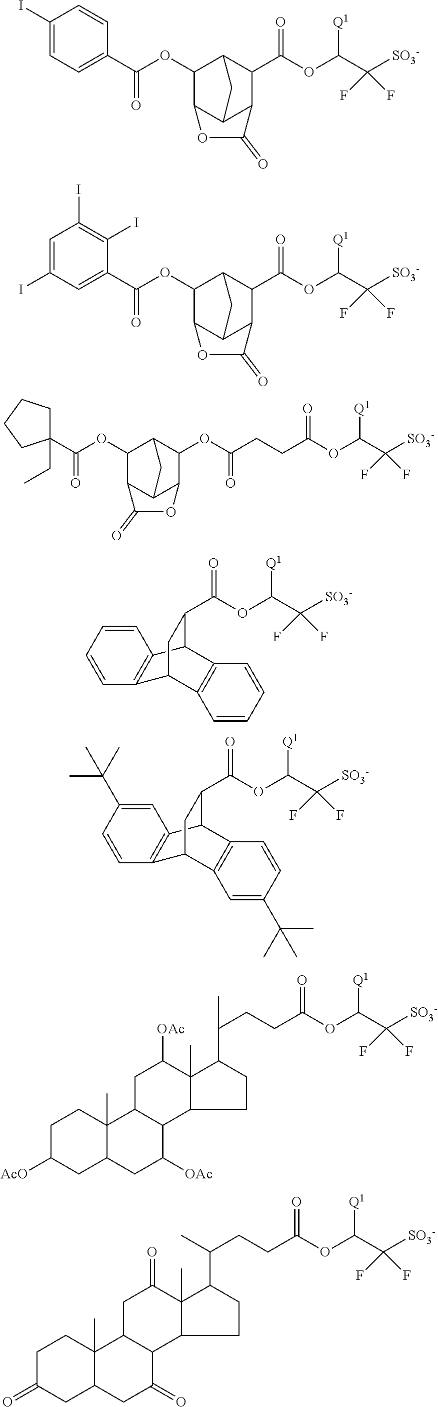

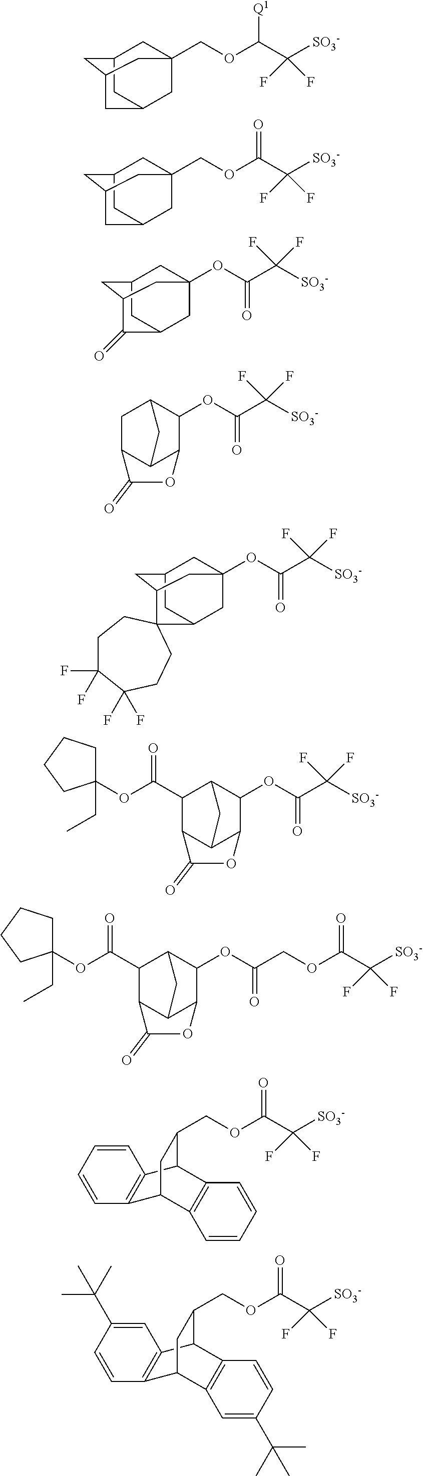

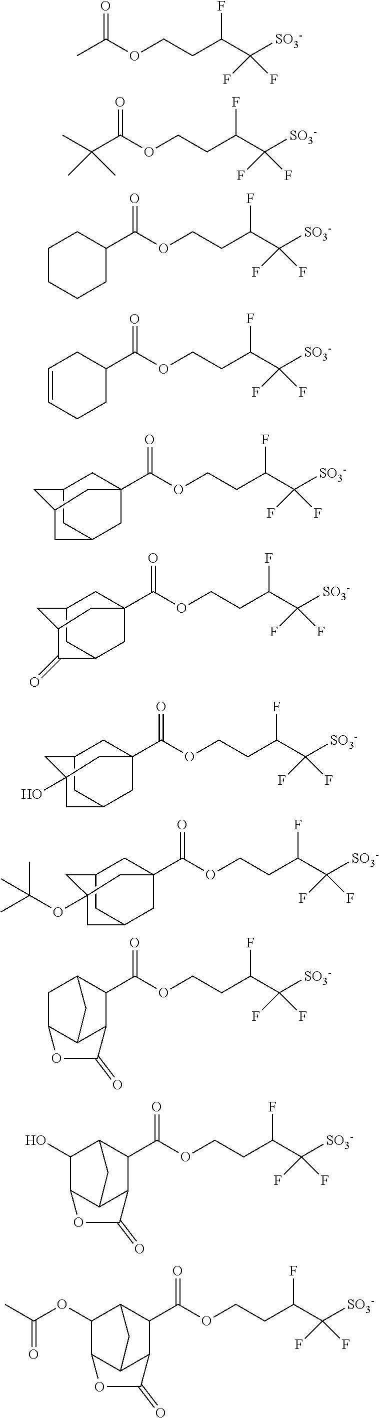

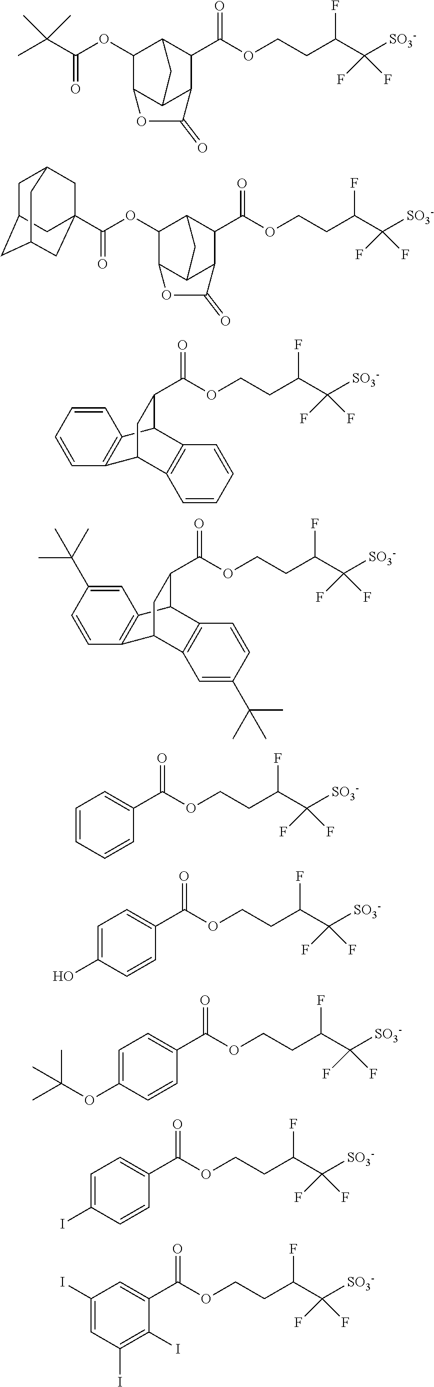

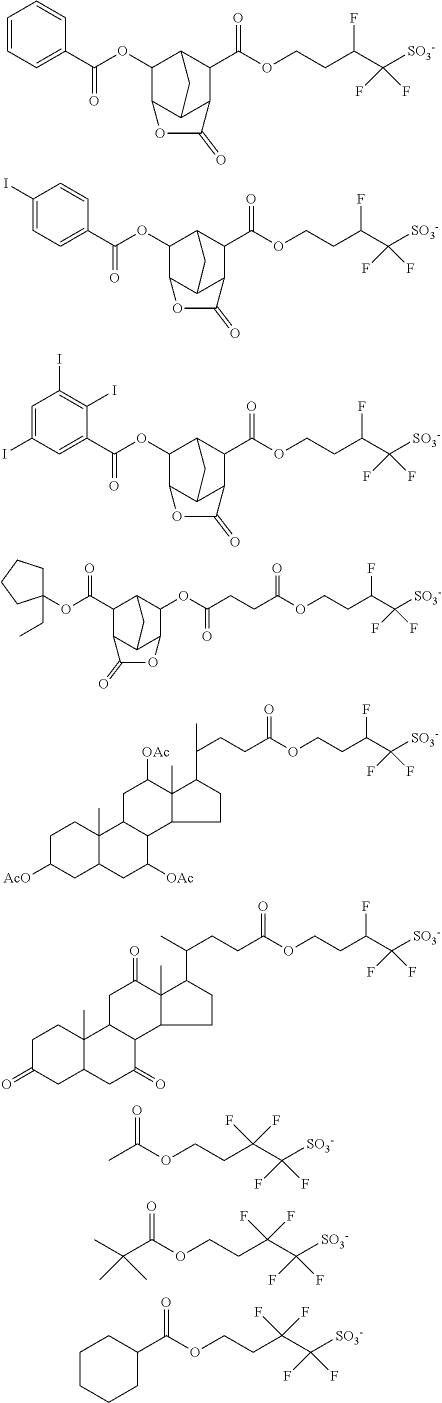

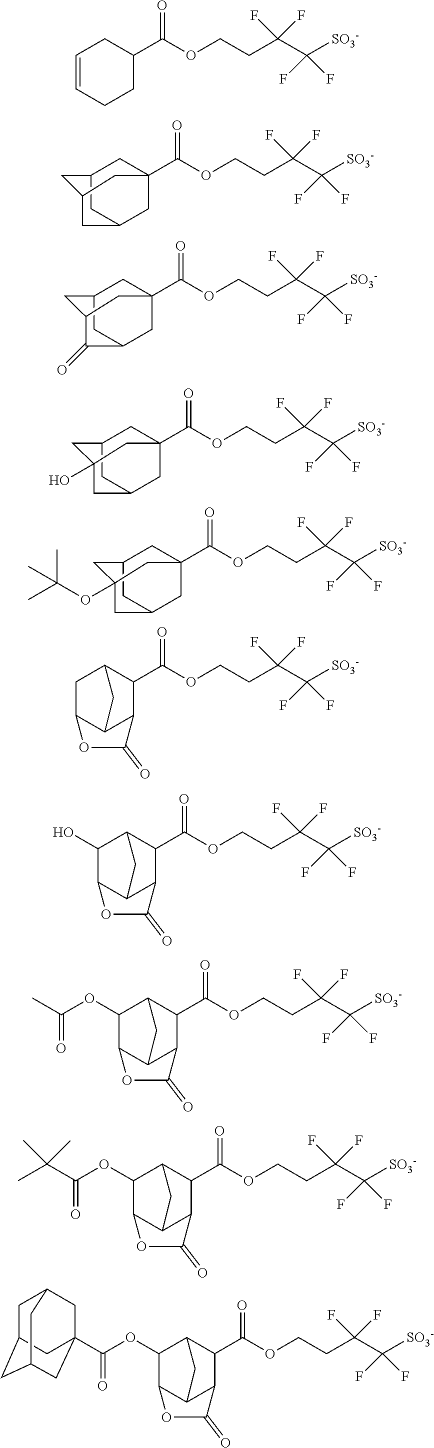

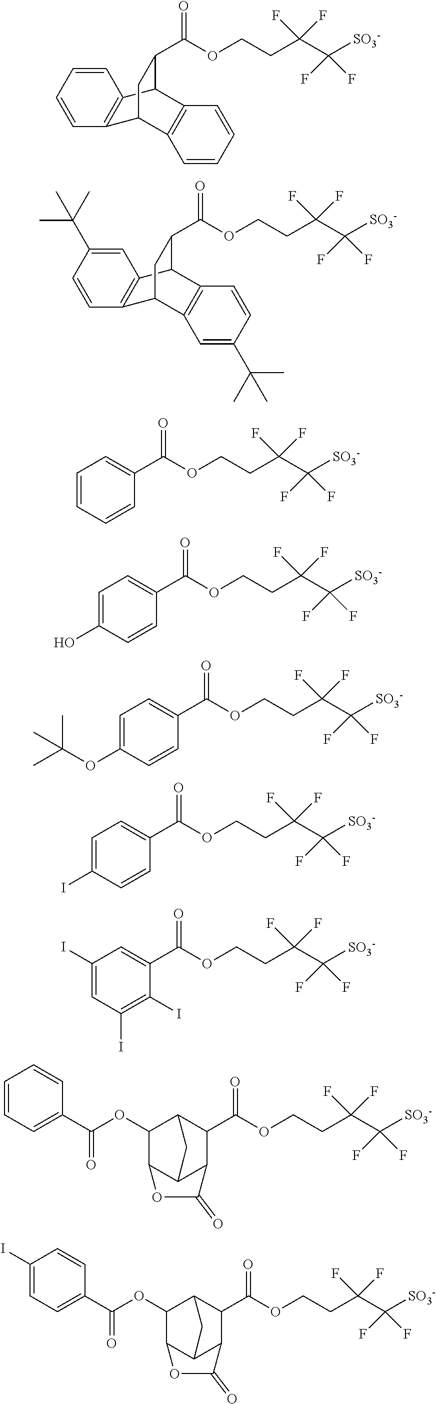

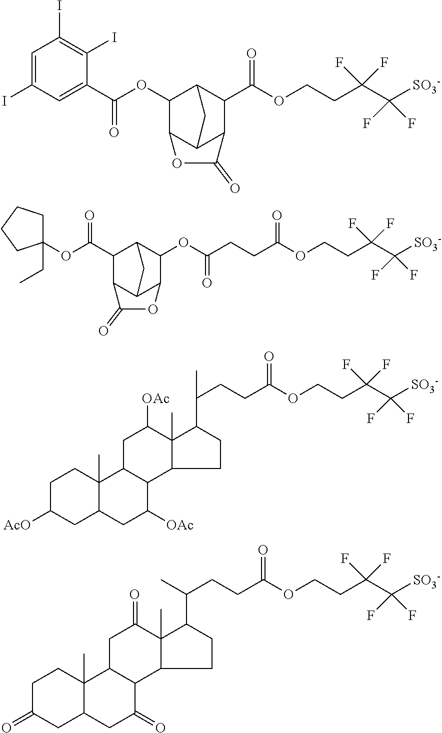

In formula (c1-2), R^{fb1} and R^{fb2} are each independently a fluorine atom or a C₁-C₄₀ hydrocarbyl group which may contain a heteroatom. The hydrocarbyl group May be saturated or unsaturated and straight, branched, or cyclic. Specific examples thereof are as exemplified above for the hydrocarbyl group R^{fa1} in formula (c1-1-1). R^{fb1} and R^{fb2} are preferably a fluorine atom or a C₁-C₄ straight fluorinated alkyl group. Also, R^{fb1} and R^{fb2} may bond together to form a ring with the linkage: —CF₂—SO₂—N⁻—SO₂—CF₂—to which they are attached, and in this case, the group obtained by bonding R^{fb1} and R^{fb2} together is preferably a fluorinated ethylene group or a fluorinated propylene group.

In formula (c1-3), R^{fc1}, R^{fc2}, and R^{fc3} are each independently a fluorine atom or a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group R^{fa1} in formula (c1-1-1). R^{fc1}, R^{fc2}, and R^{fc3} are preferably a fluorine atom or a C₁-C₄ straight fluorinated alkyl group. Also, R^{fc1} and R^{fc2} may bond together to form a ring with the linkage: —CF₂—SO₂—C⁻—SO₂—CF₂—to which they are attached, and in this case, the group obtained by bonding R^{fc1} and R^{fc2} together is preferably a fluorinated ethylene group or a fluorinated propylene group.

In formula (c1-4), R^{fd} is a C₁-C₄₀ hydrocarbyl group which may contain a heteroatom. The hydrocarbyl group may be saturated or unsaturated and straight, branched, or cyclic. Specific examples thereof are as exemplified above for the hydrocarbyl group R^{fa1} in formula (c1-1-1).

Examples of the anion having formula (c1-4) are shown below, but not limited thereto.

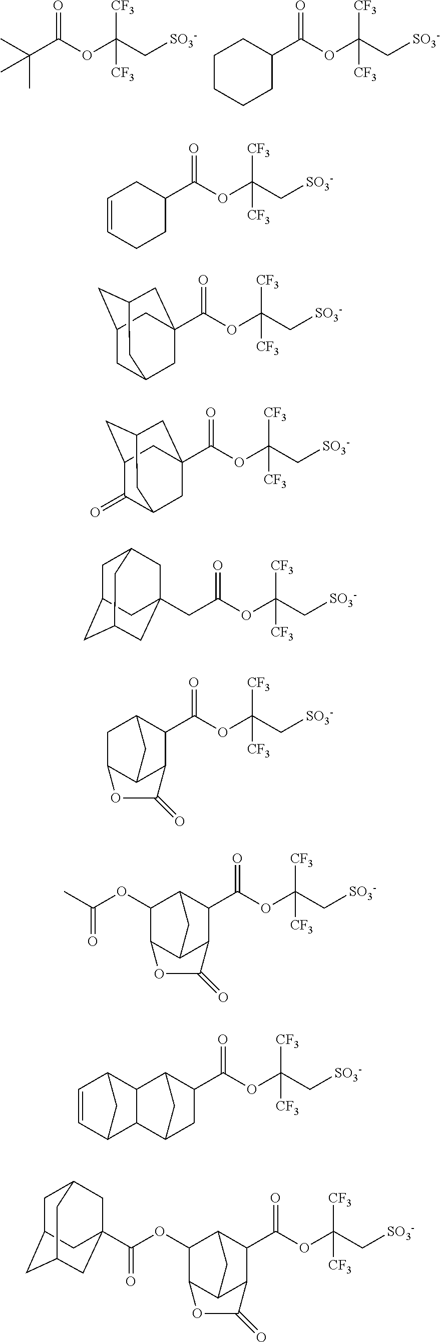

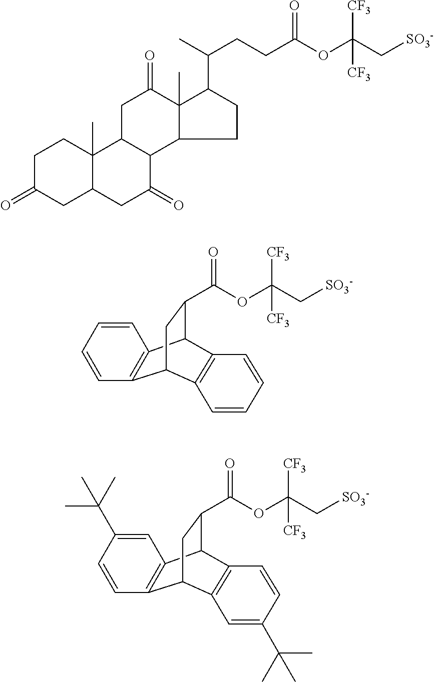

Examples of the non-nucleophilic counter ion further include an anion having an aromatic ring substituted with an iodine atom or a bromine atom. Specific examples of the anion include those having the formula (c1-5).

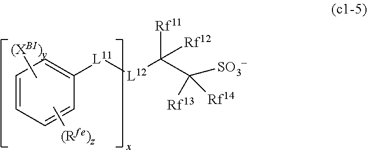

In formula (c1-5), x is 1, 2, or 3. y is 1, 2, 3, 4, or 5. z is 0, 1, 2, or 3. Provided that 1≤y+z≤5. y is preferably 1, 2, or 3, more preferably 2 or 3. z is preferably 0, 1, or 2.

In formula (c1-5), X^{BI} is an iodine atom or a bromine atom, and may be identical or different when x and/or y is 2 or more.

In formula (c1-5), L¹¹ is a single bond, an ether bond, an ester bond, or a C₁-C₆ saturated hydrocarbylene group which may contain an ether bond or an ester bond. The saturated hydrocarbylene group may be straight, branched, or cyclic.

In formula (c1-5), L¹² is a single bond or a C₁-C₂₀ divalent linking group when x is 1, and is a C₁-C₂₀ (x+1)-valent linking group when x is 2 or 3, and the linking group may contain an oxygen atom, a sulfur atom, or a nitrogen atom.

In formula (c1-5), R^{fe} is a hydroxy group, a carboxy group, a fluorine atom, a chlorine atom, a bromine atom, an amino group, or a C₁-C₂₀ hydrocarbyl group, a C₁-C₂₀ hydrocarbyloxy group, a C₂-C₂₀ hydrocarbylcarbonyl group, a C₂-C₂₀ hydrocarbyloxycarbonyl group, a C₂-C₂₀ hydrocarbylcarbonyloxy group, or a C₁-C₂₀ hydrocarbylsulfonyloxy group, which may contain a fluorine atom, a chlorine atom, a bromine atom, a hydroxy group, an amino group, or an ether bond, or —N(R^{feA})(R^{feB}), —N(R^{feC})—C(═O)—R^{feD}, or —N(R^{feC})—C(═O)—O—R^{feD}. R^{feA} to R^{feB} are each independently a hydrogen atom or a C₁-C₆ saturated hydrocarbyl group. R^{feC} is a hydrogen atom or a C₁-C₆ saturated hydrocarbyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{feD} is a C₁-C₁₆ aliphatic hydrocarbyl group, a C₆-C₁₂ aryl group, or a C₇-C₁₅ aralkyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched, or cyclic. The hydrocarbyl group, the hydrocarbyloxy group, hydrocarbylcarbonyl group, the hydrocarbyloxycarbonyl group, the hydrocarbylcarbonyloxy group, and the hydrocarbylsulfonyloxy group may be straight, branched, or cyclic. When x and/or z is 2 or more, R^{fe} may be identical or different. Inter alia, R^{fe} is preferably a hydroxy group, —N(R^{feC})—C(═O)—R^{feD}, —N(R^{feC})—C(═O)—O—R^{feD}, a fluorine atom, a chlorine atom, a bromine atom, a methyl group, a methoxy group, or the like.

In formula (c1-5), Rf¹¹ to Rf¹⁴ are each independently a hydrogen atom, a fluorine atom, or a trifluoromethyl group, and at least one of them is a fluorine atom or a trifluoromethyl group. Rf¹¹ and Rf¹², taken together, may form a carbonyl group. In particular, both Rf¹³ and Rf¹⁴ are preferably fluorine atoms.

Examples of the anion having formula (c1-5) are shown below, but not limited thereto. Herein X^{BI} is as defined above.

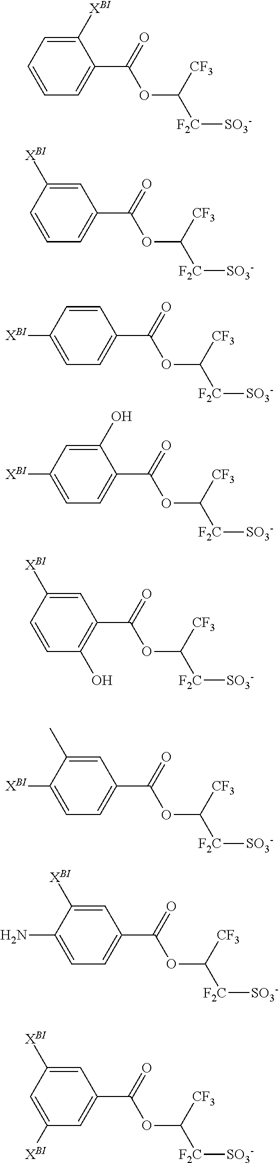

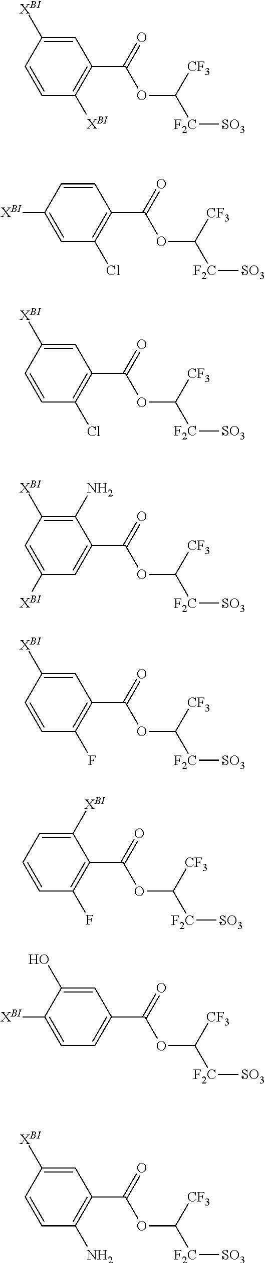

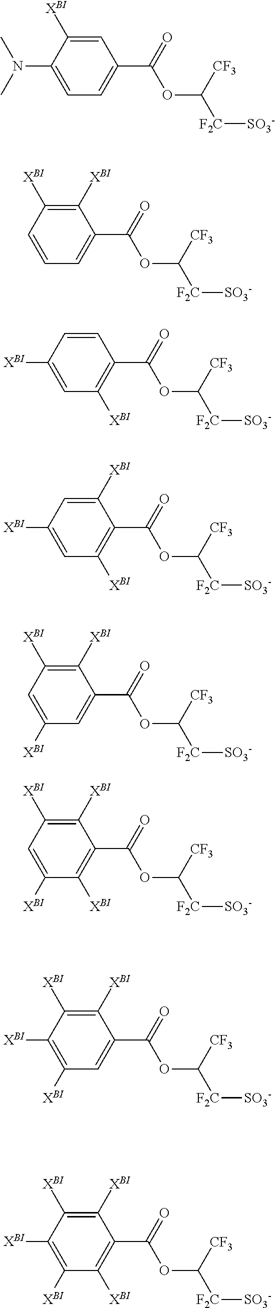

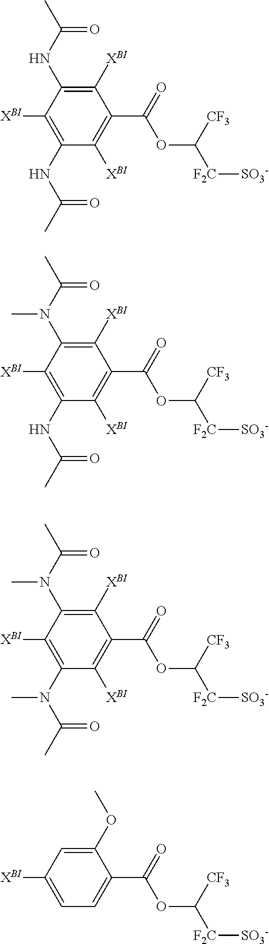

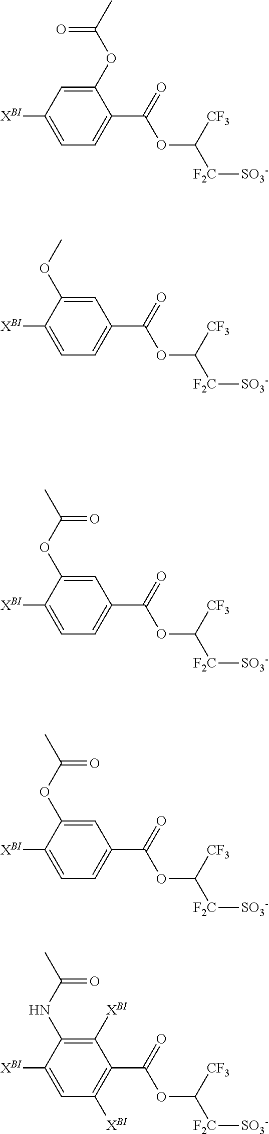

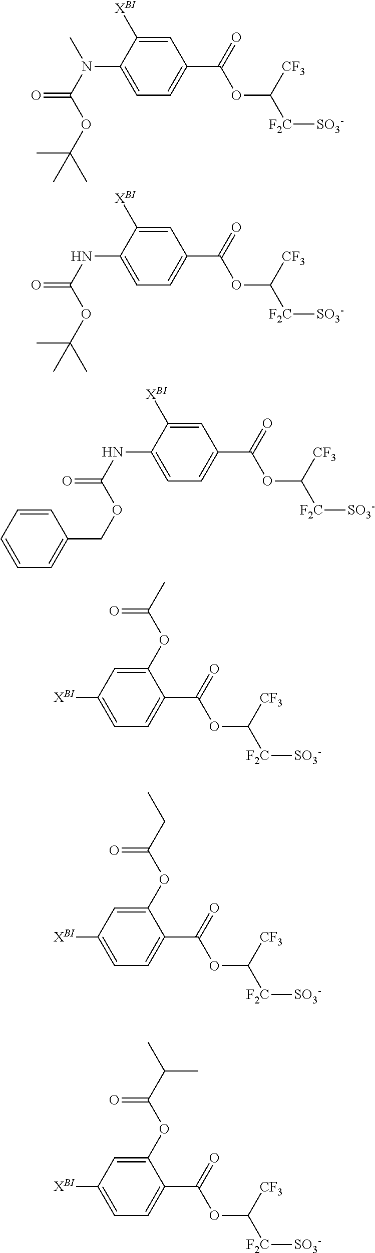

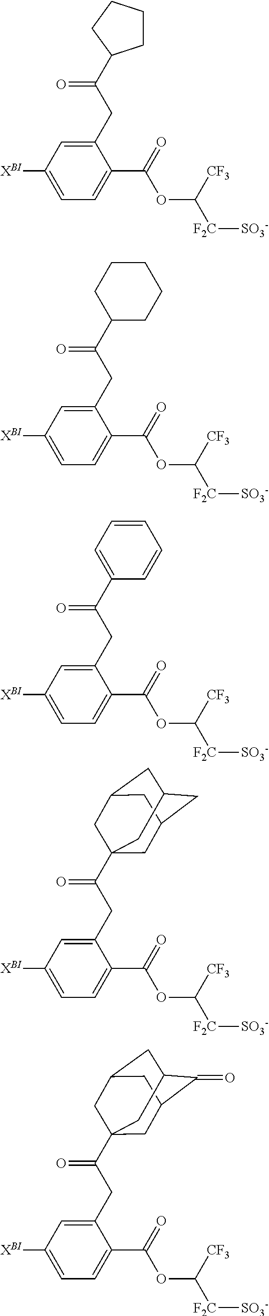

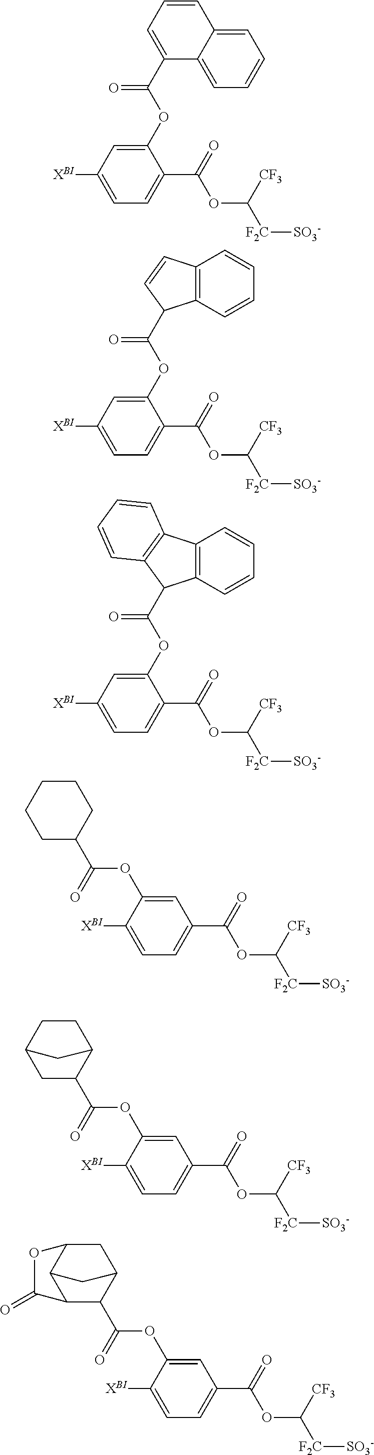

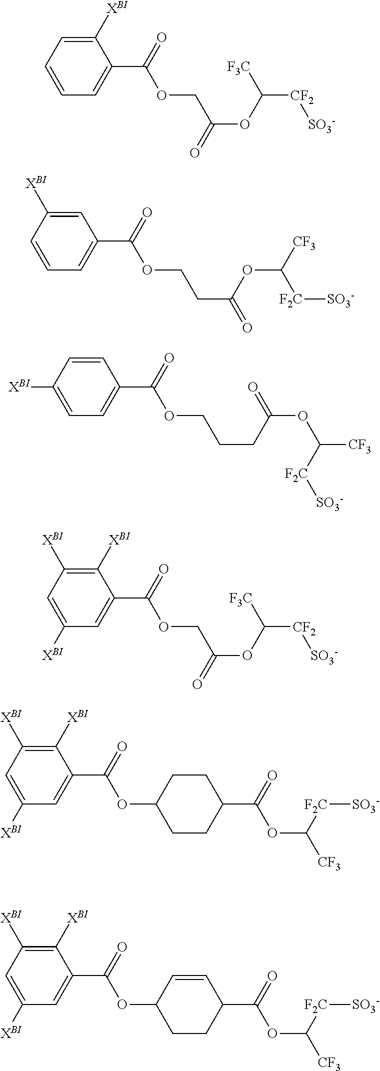

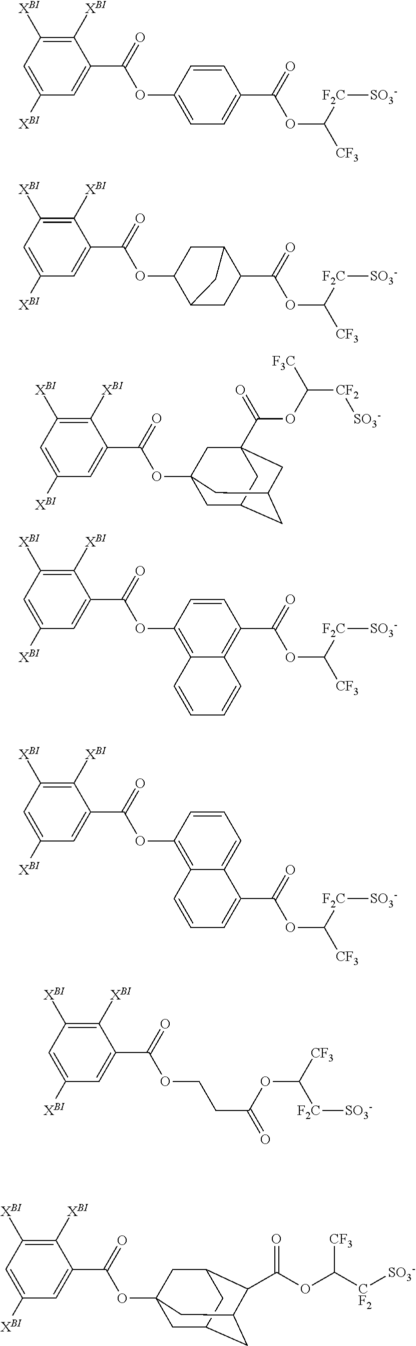

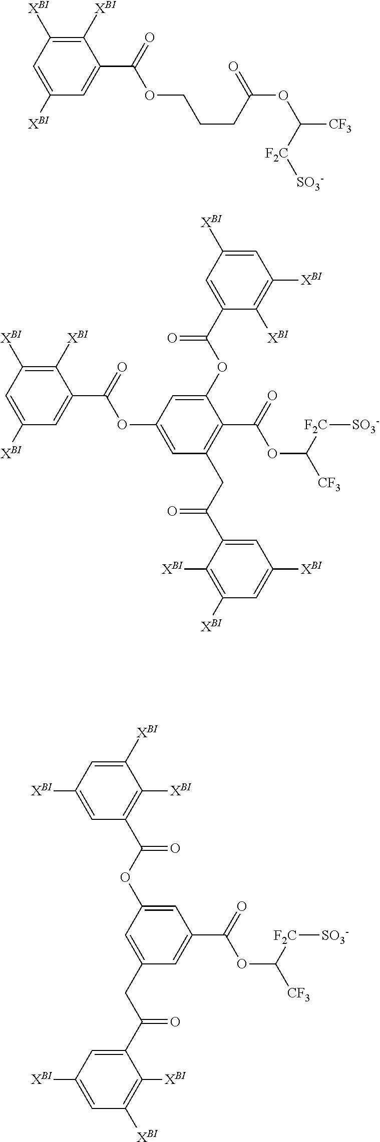

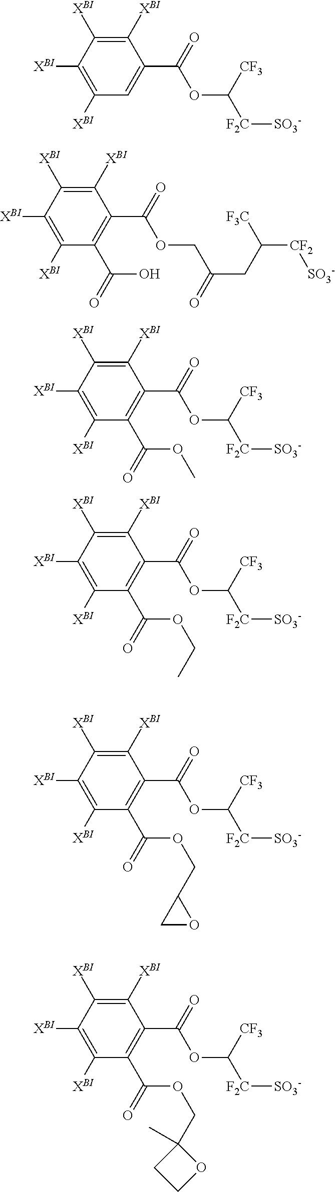

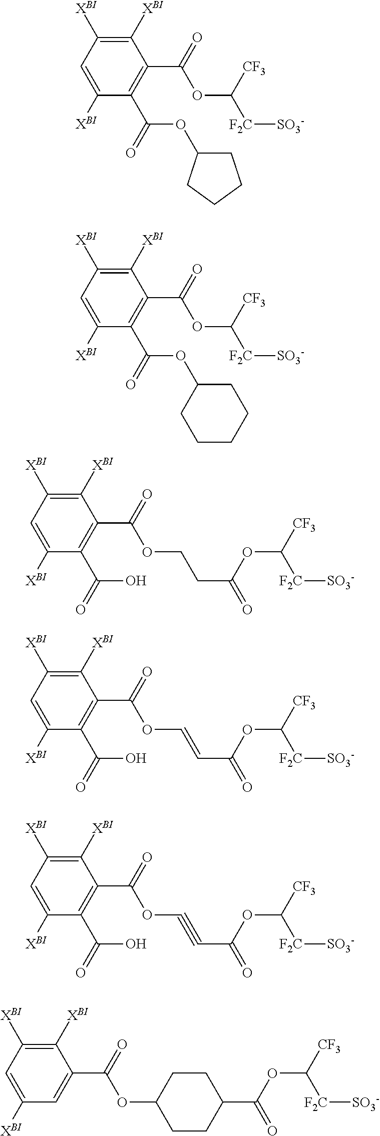

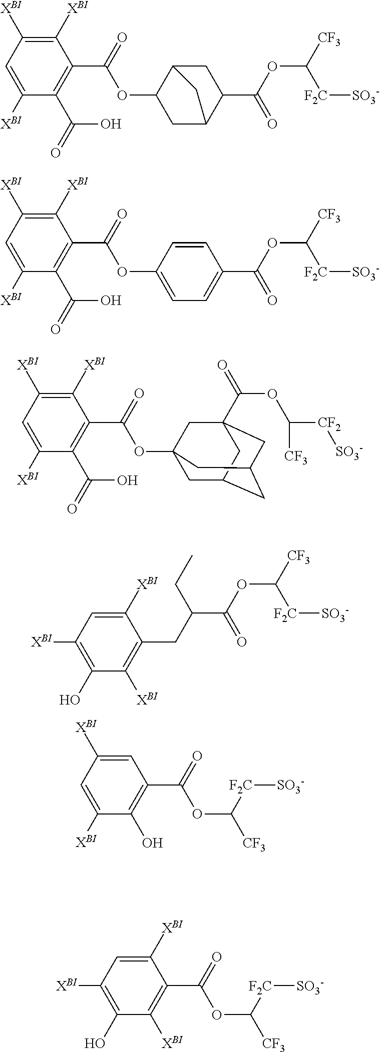

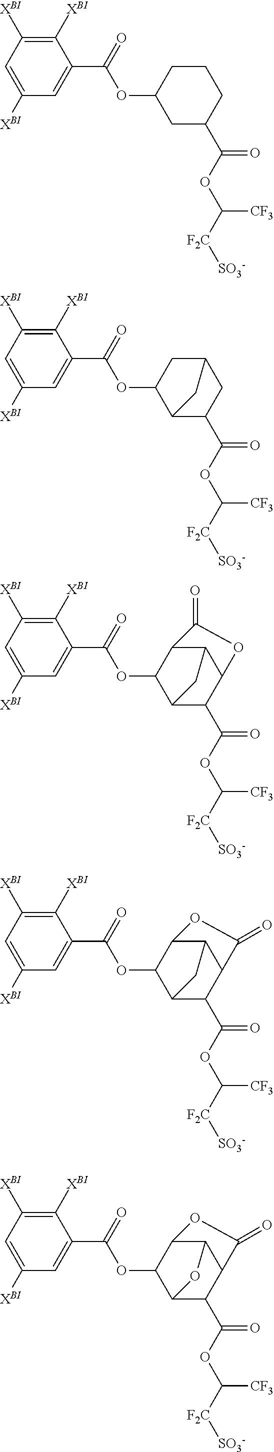

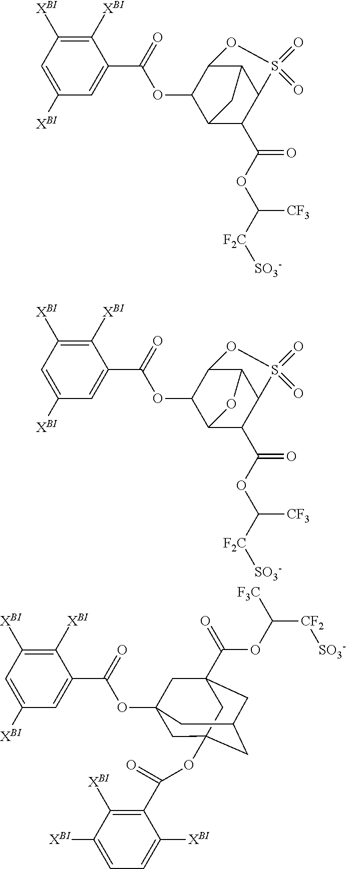

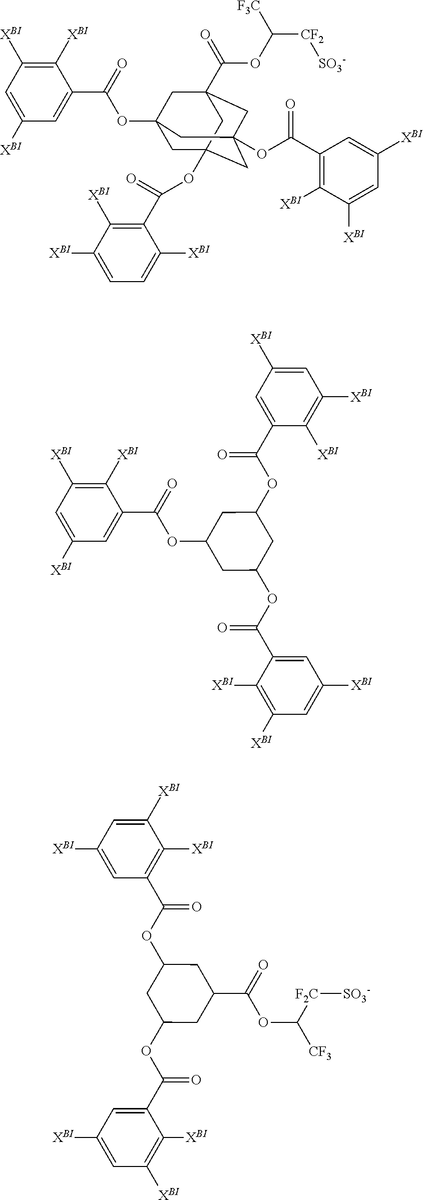

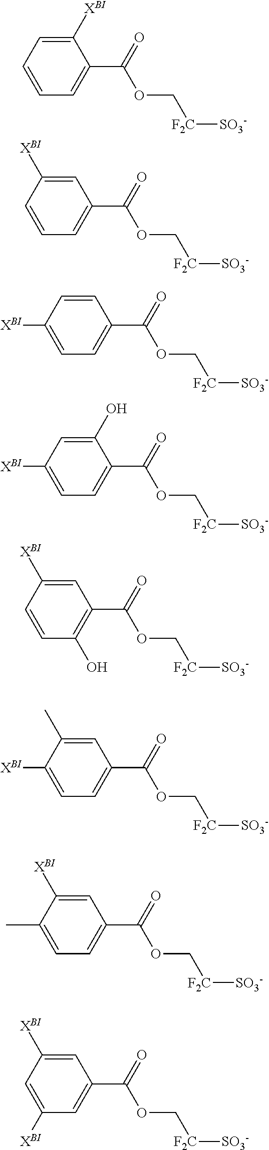

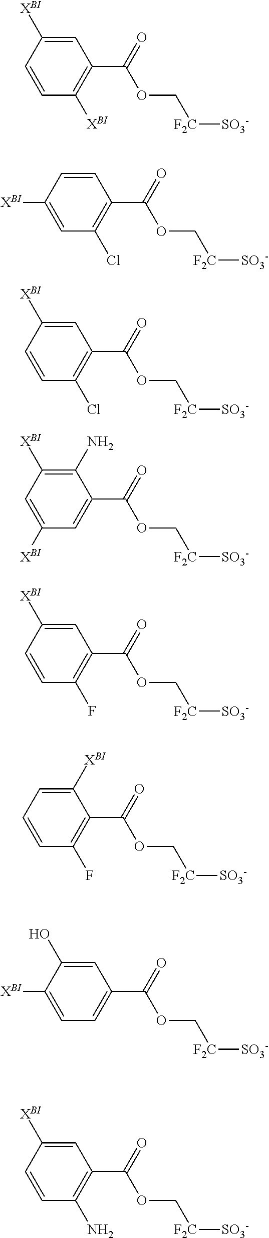

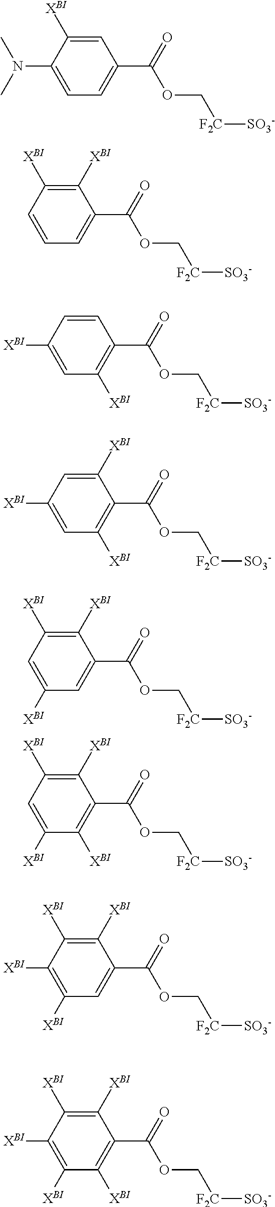

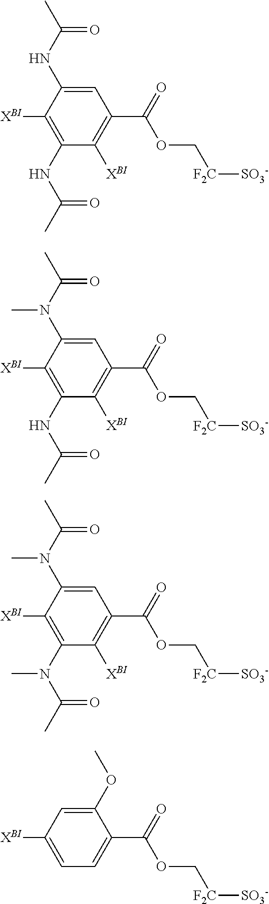

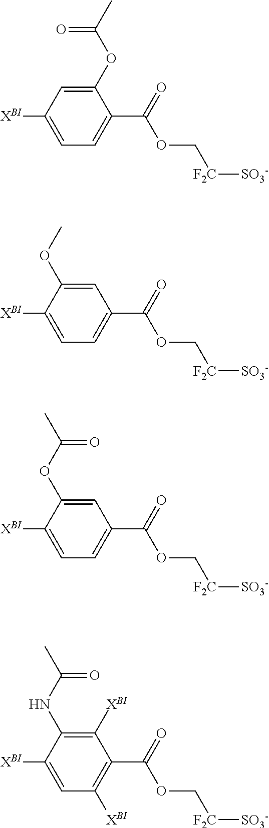

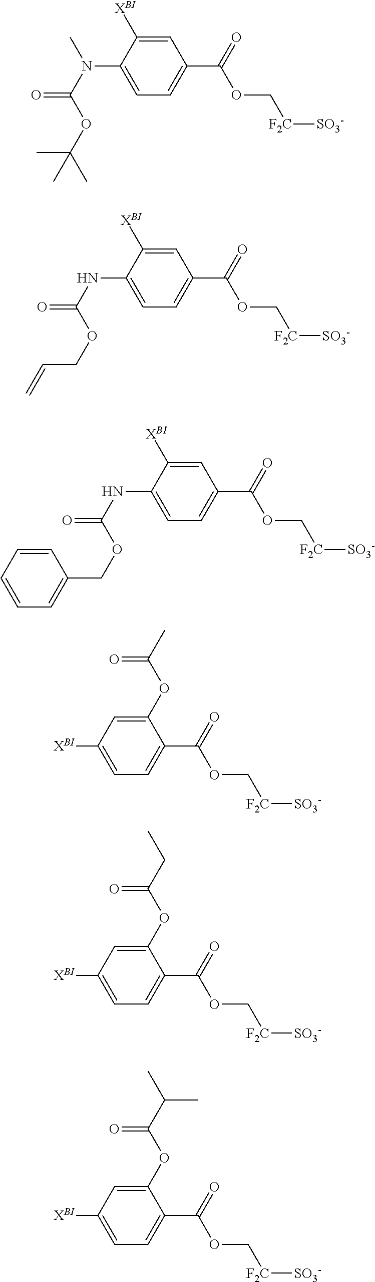

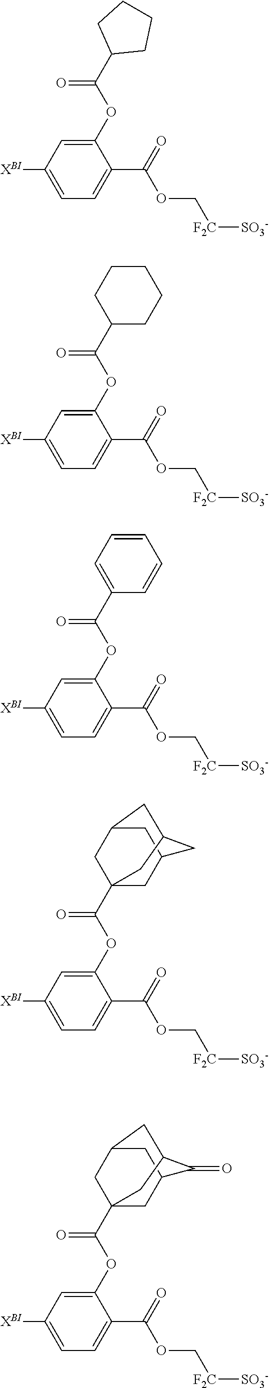

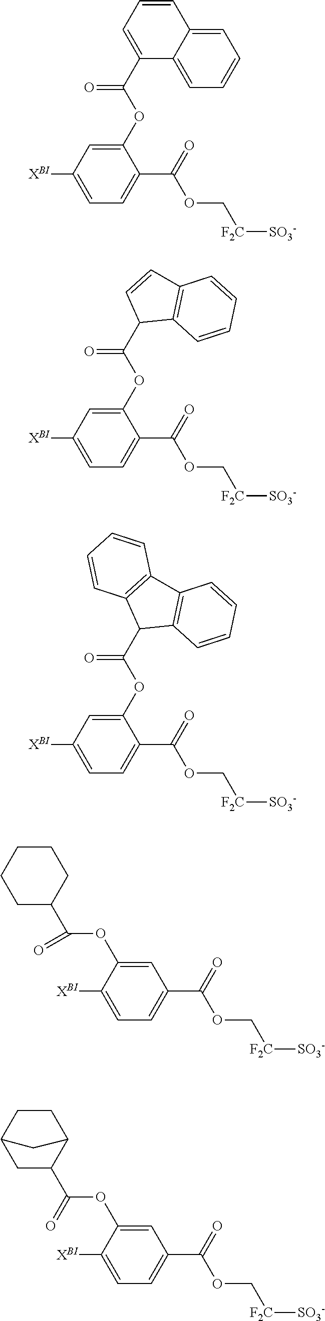

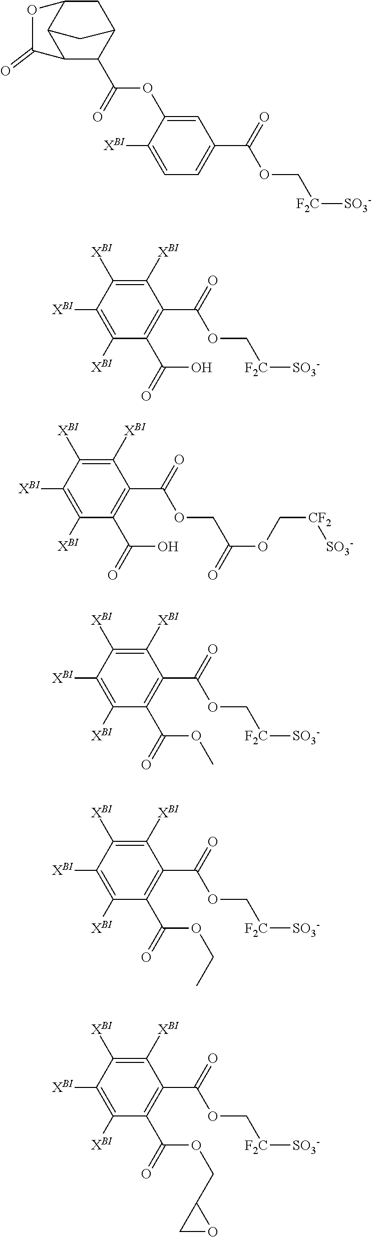

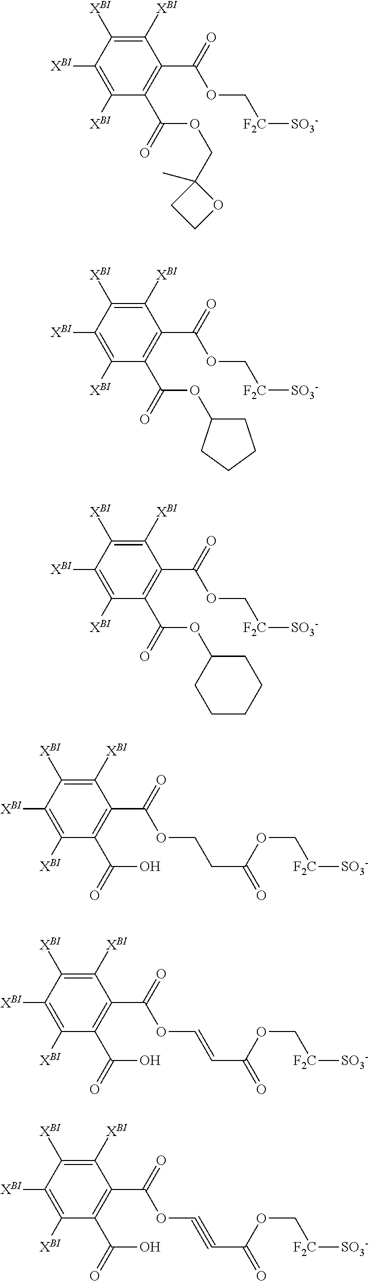

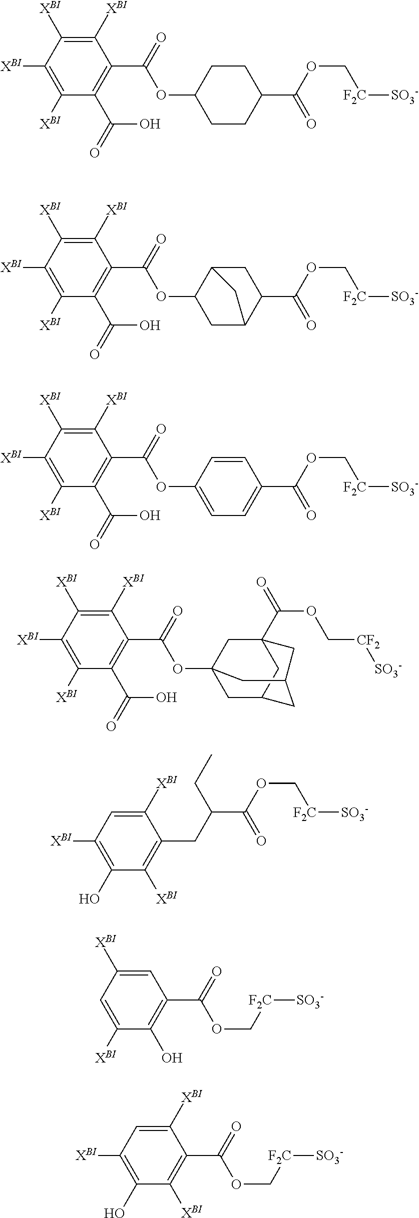

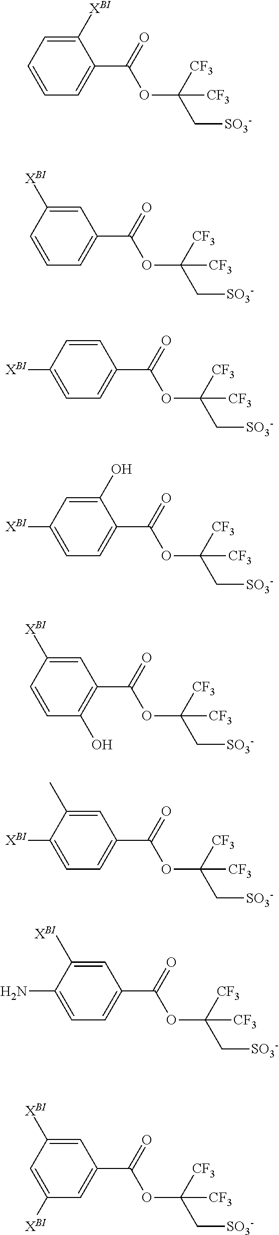

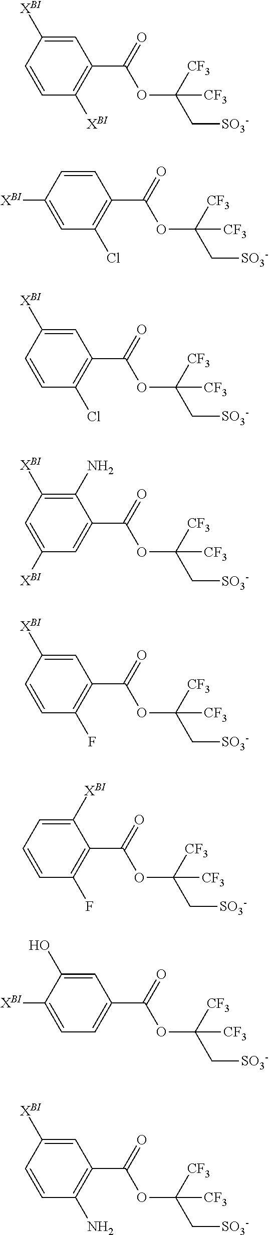

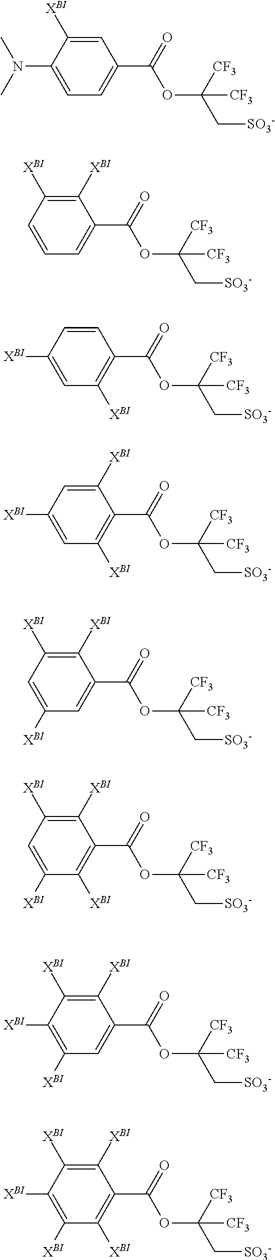

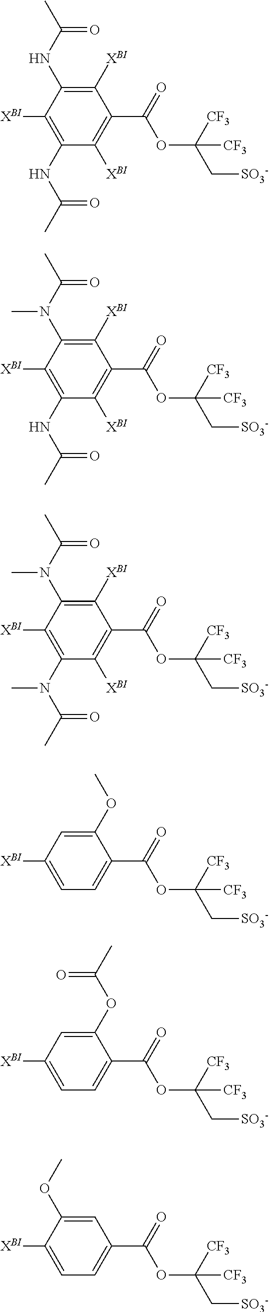

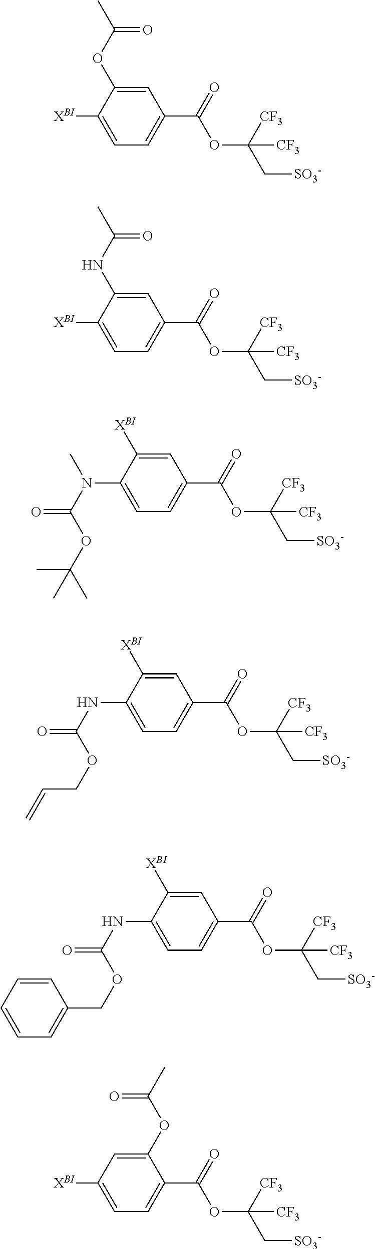

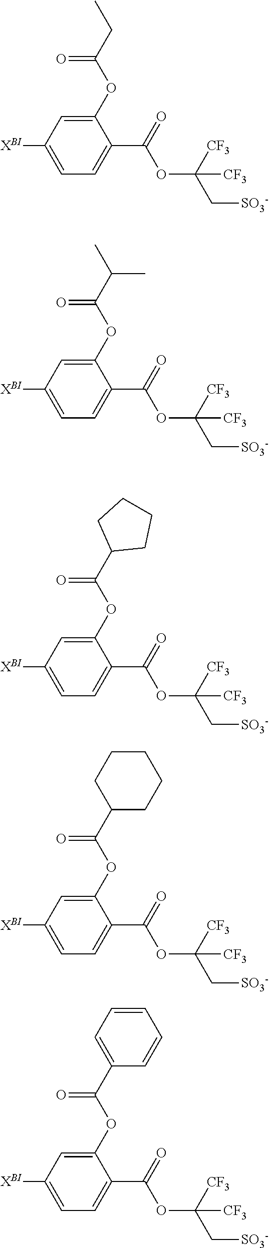

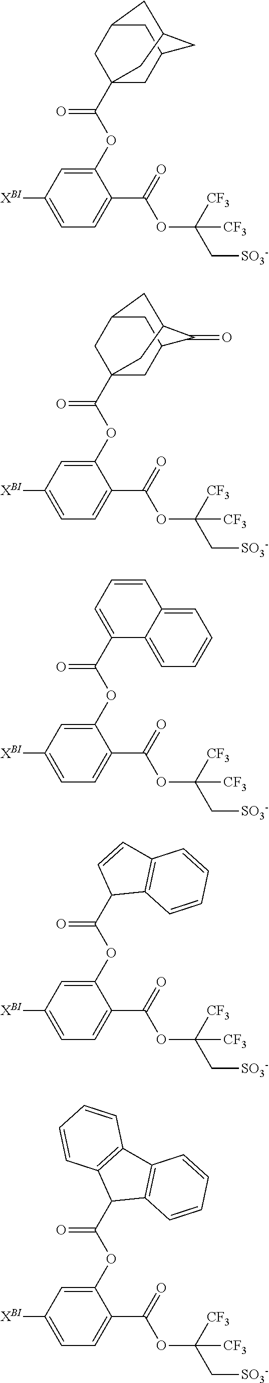

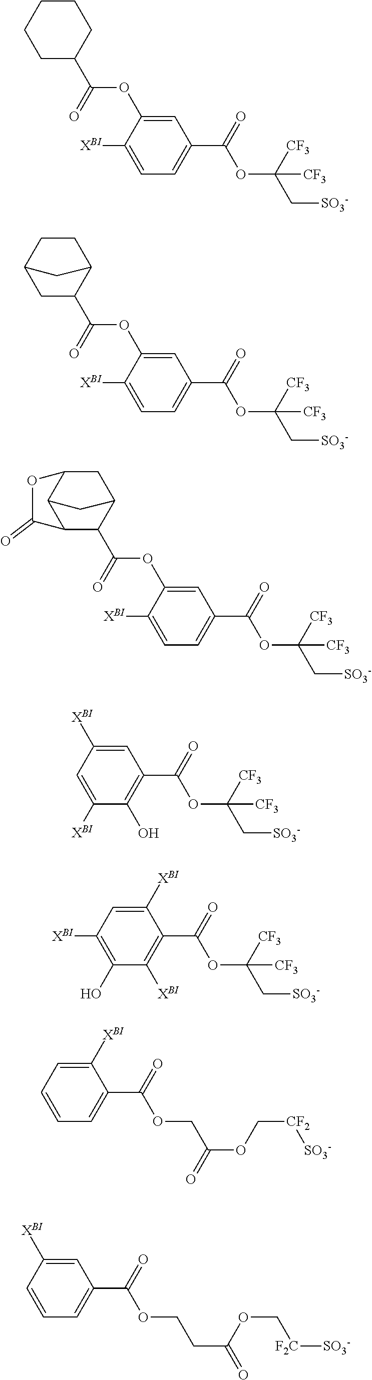

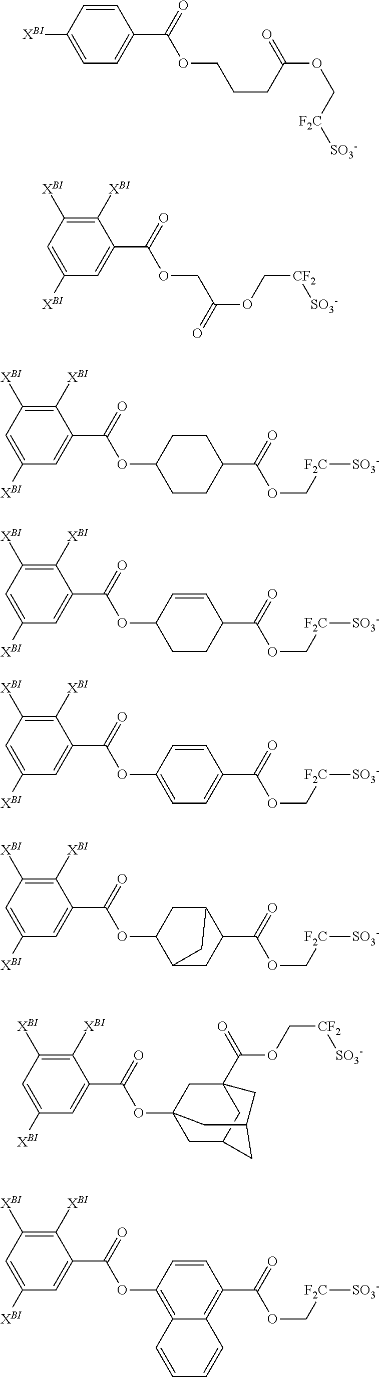

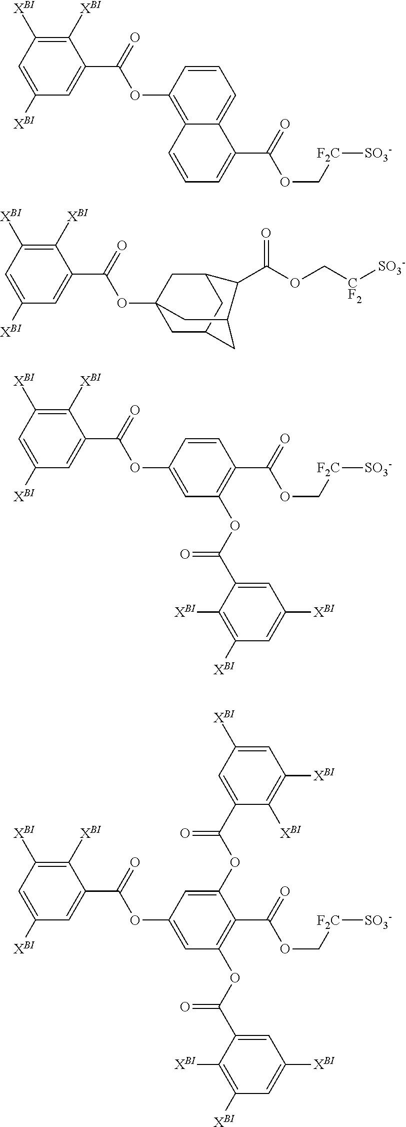

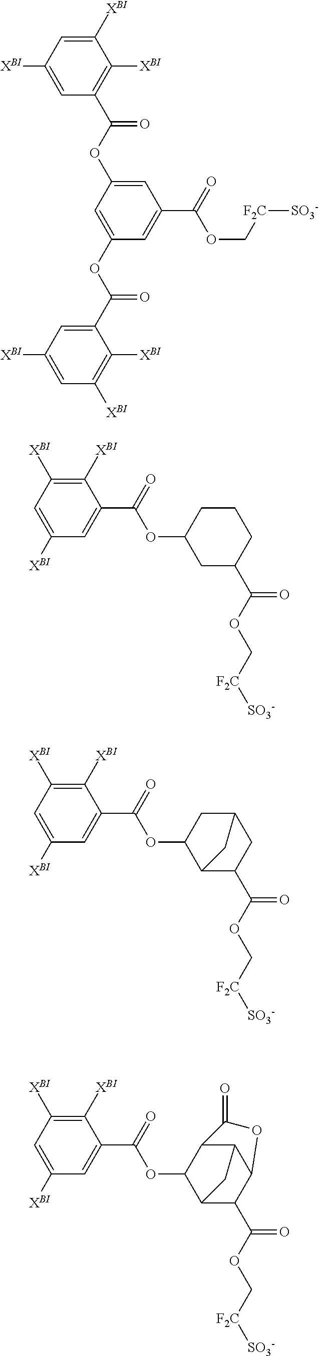

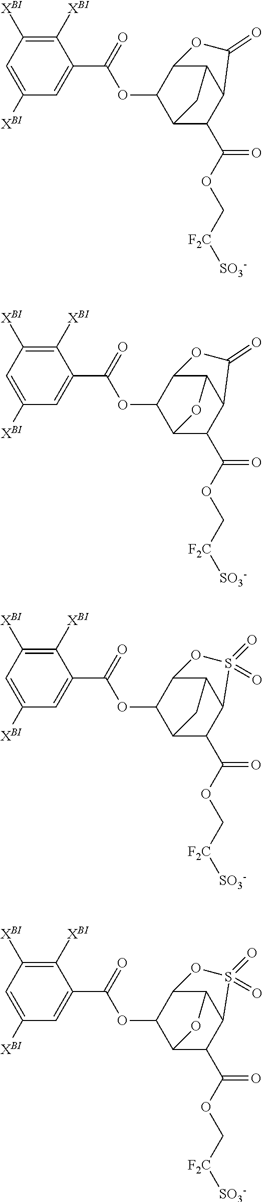

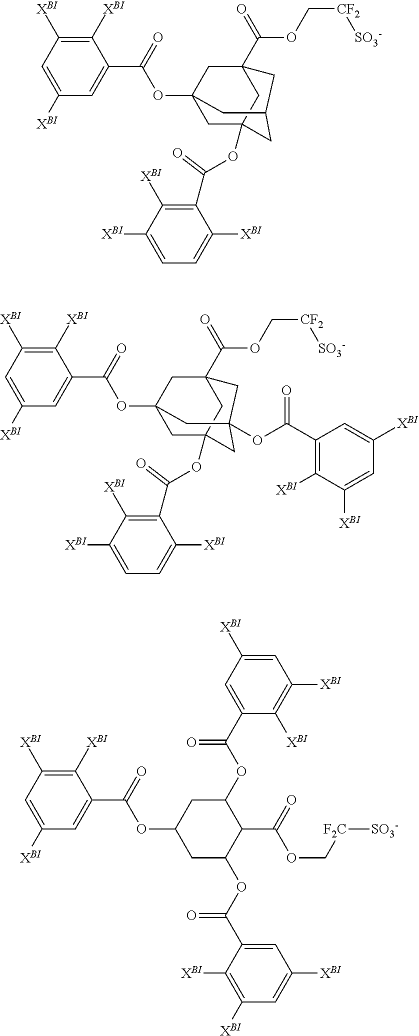

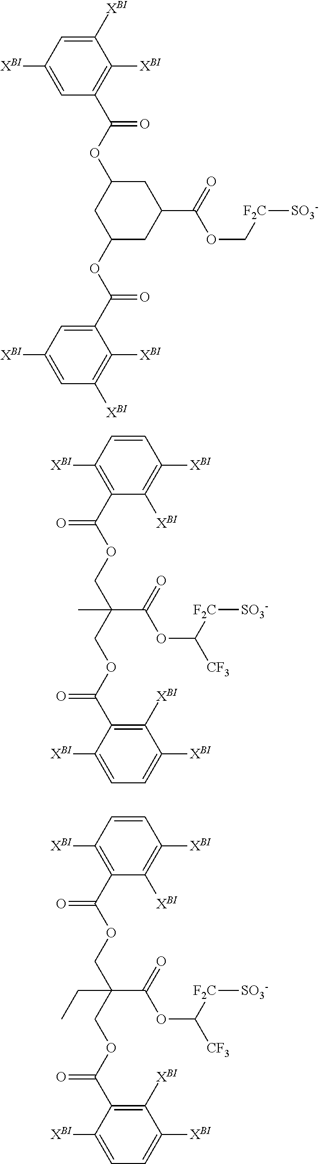

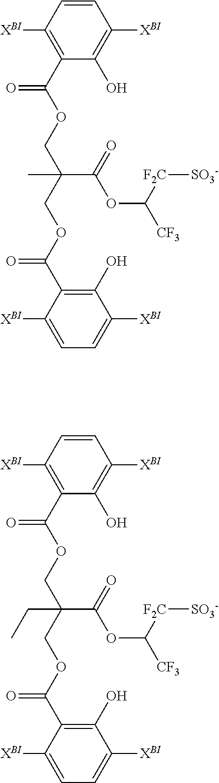

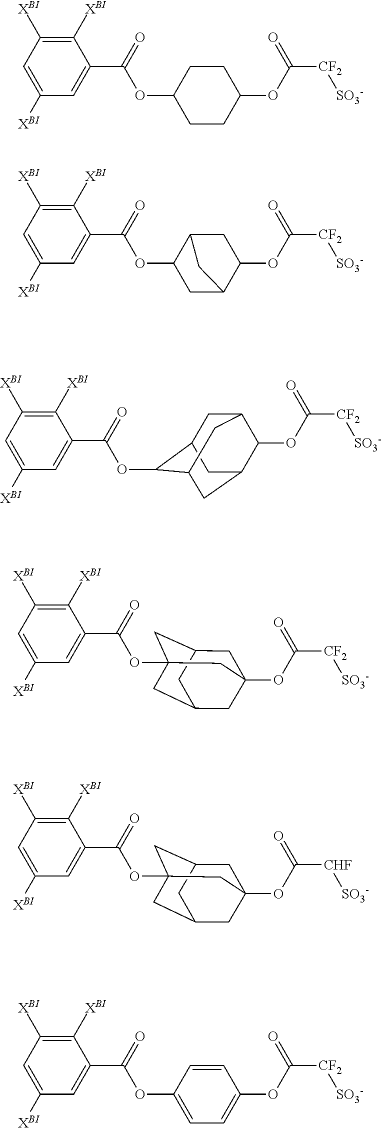

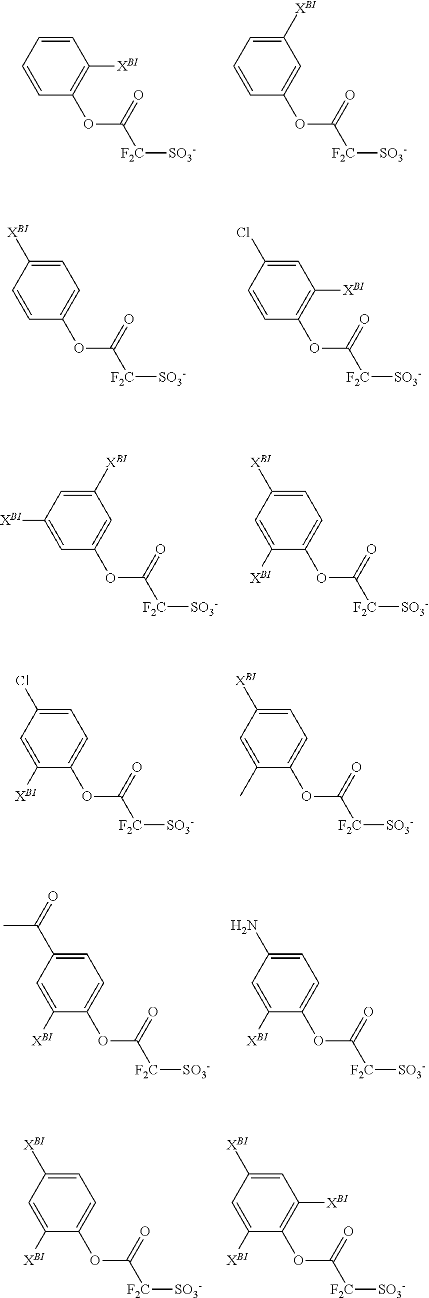

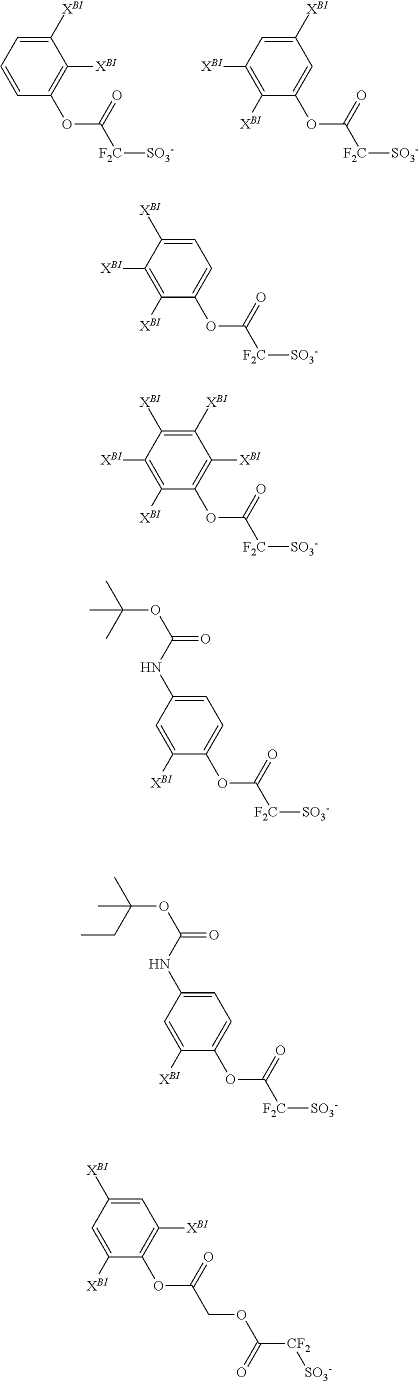

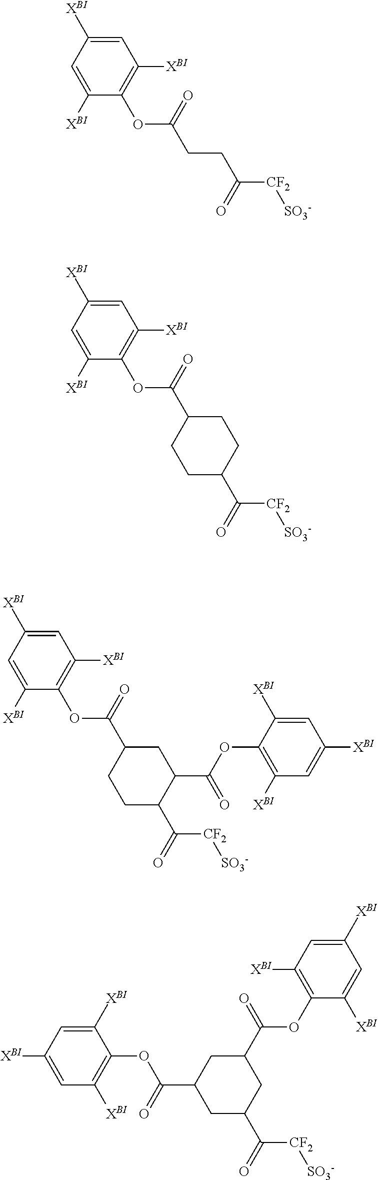

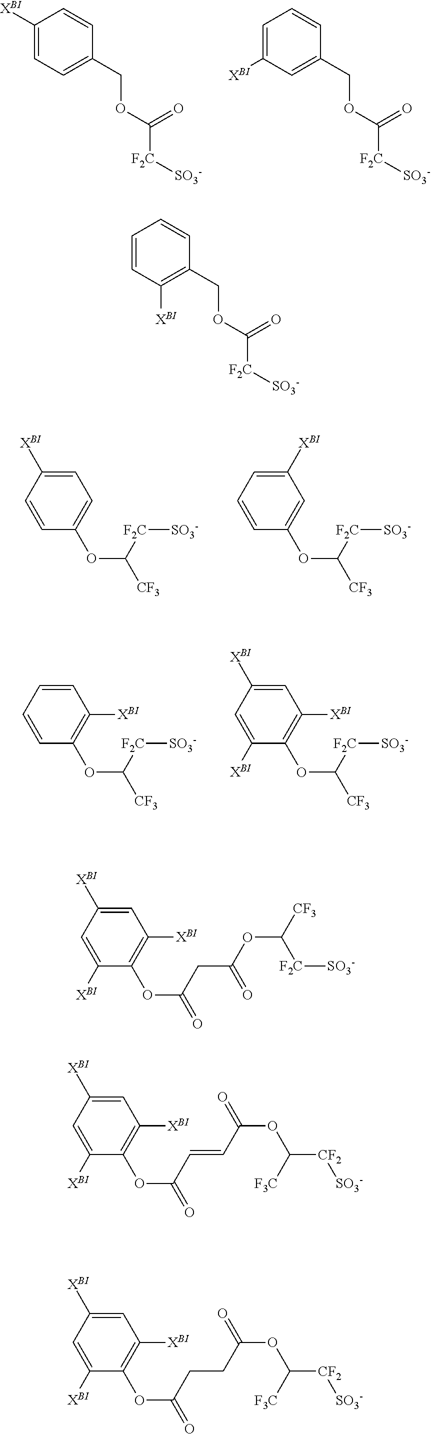

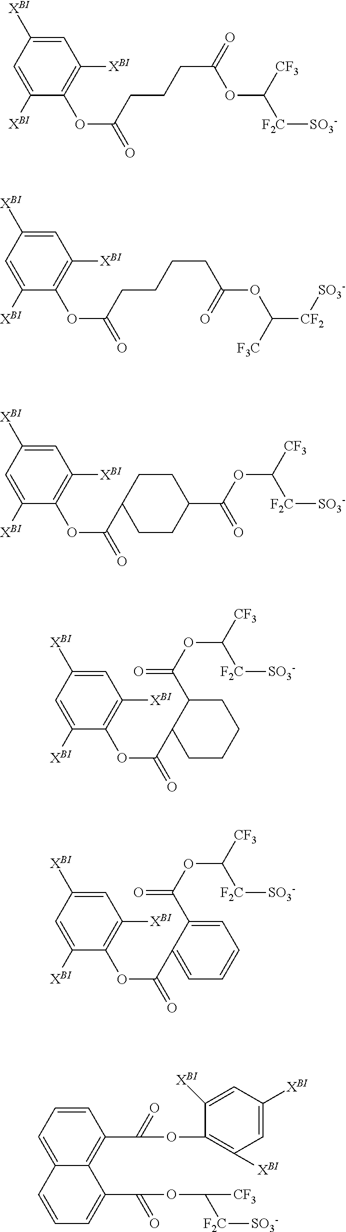

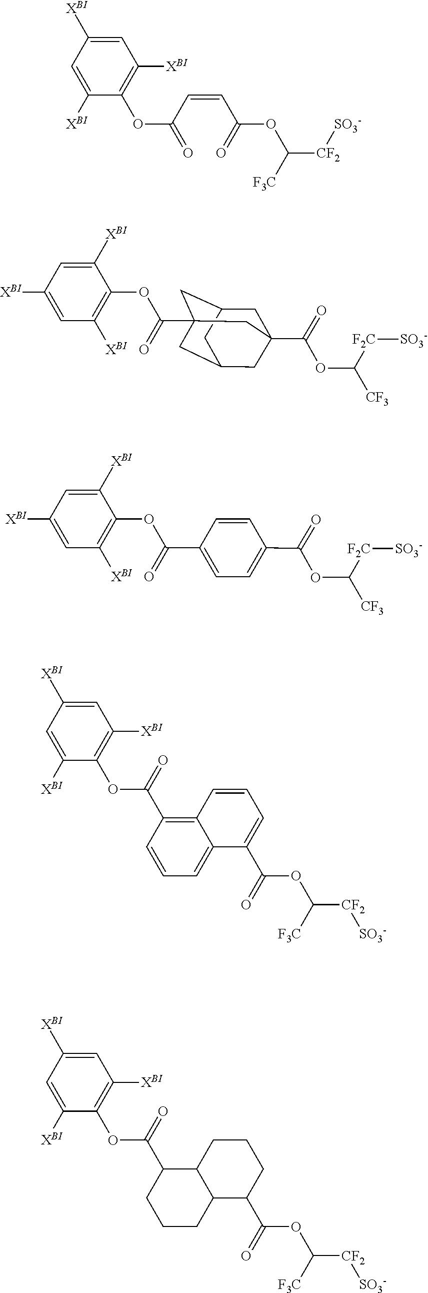

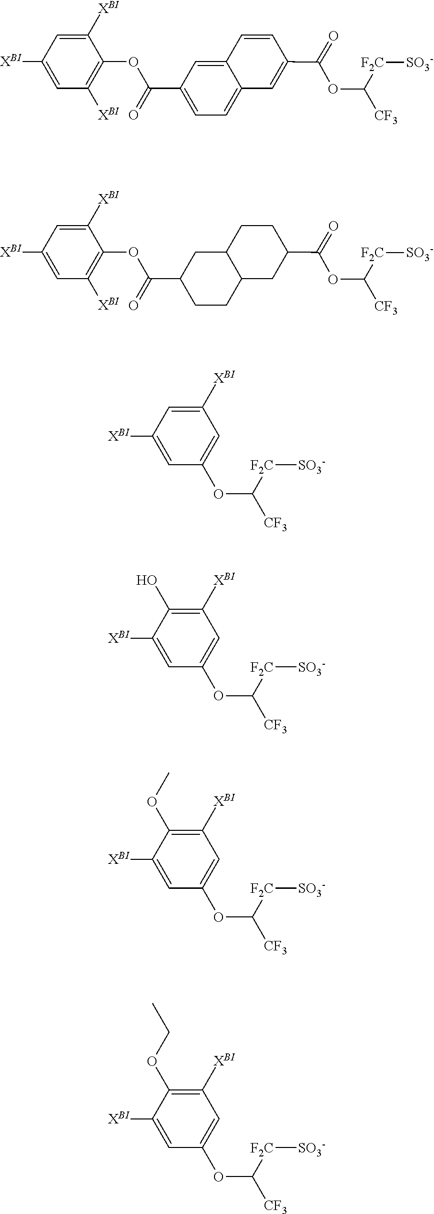

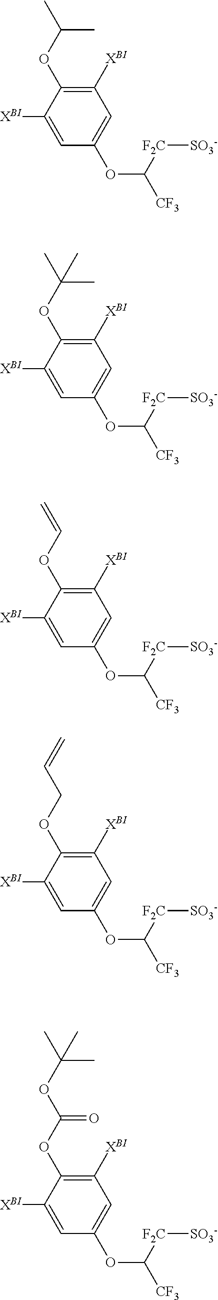

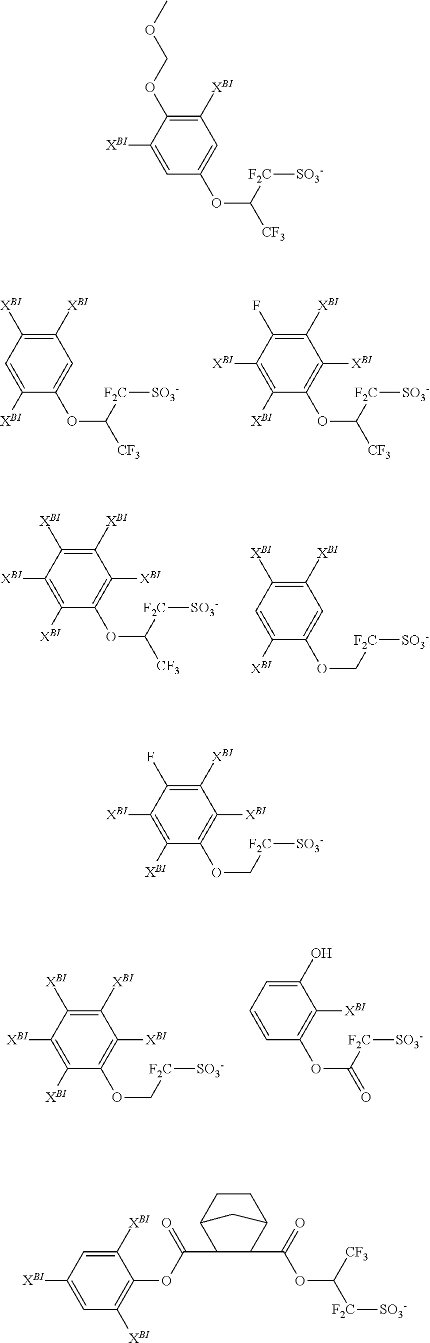

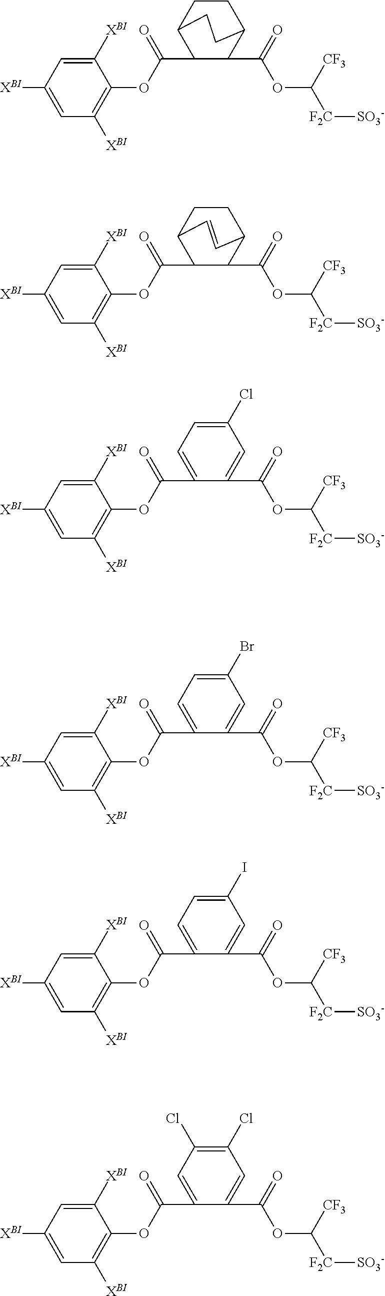

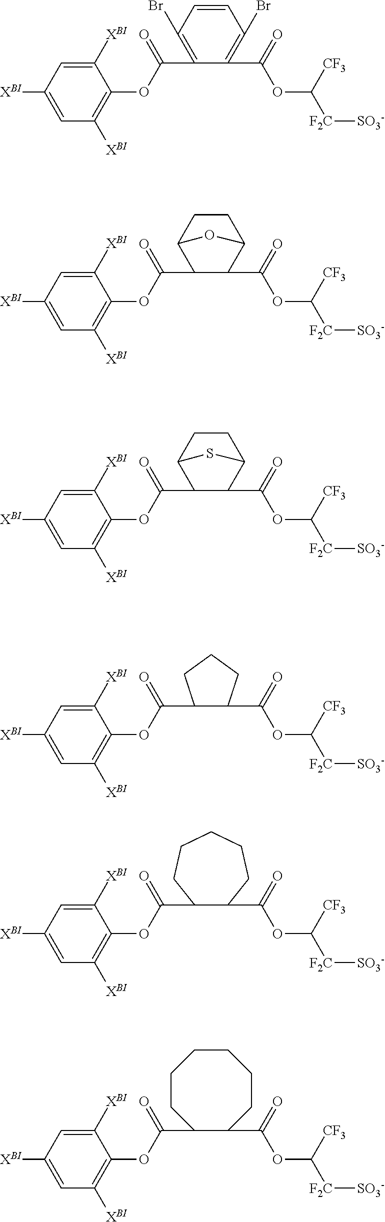

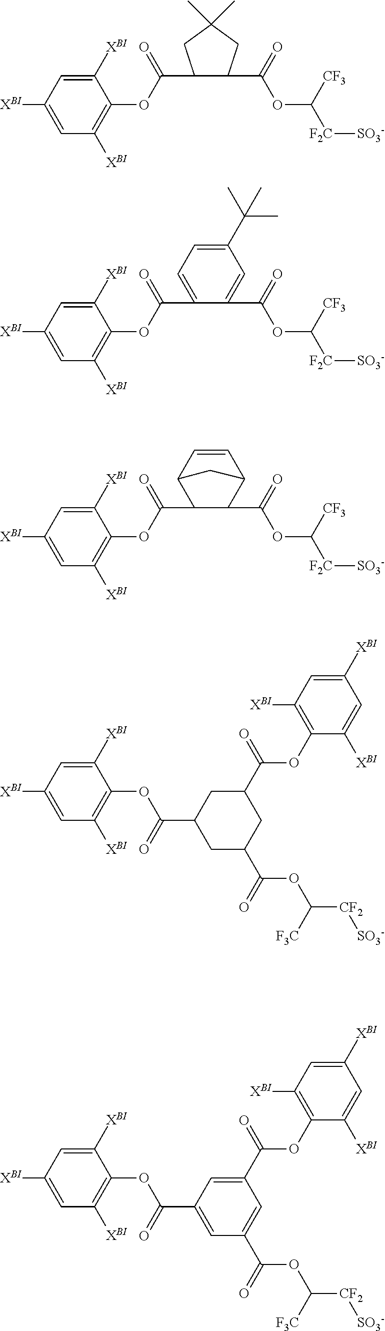

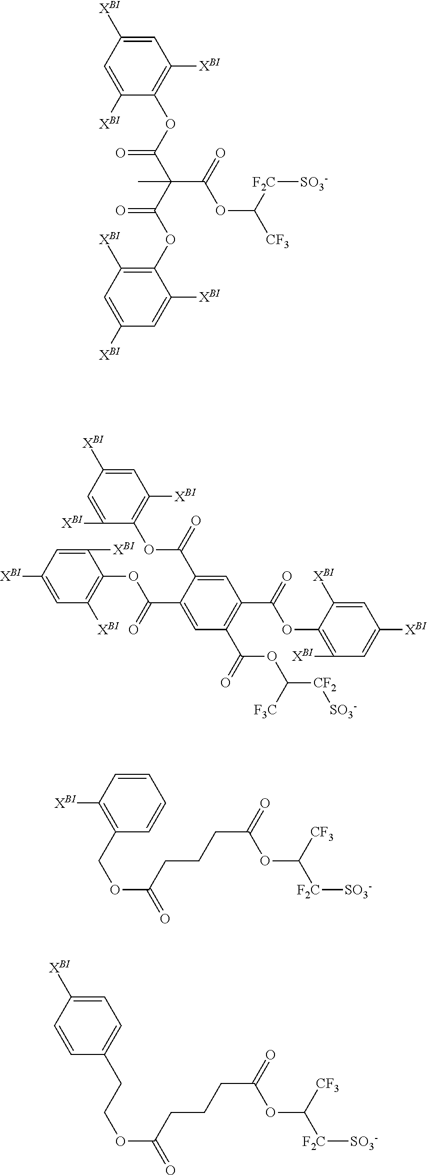

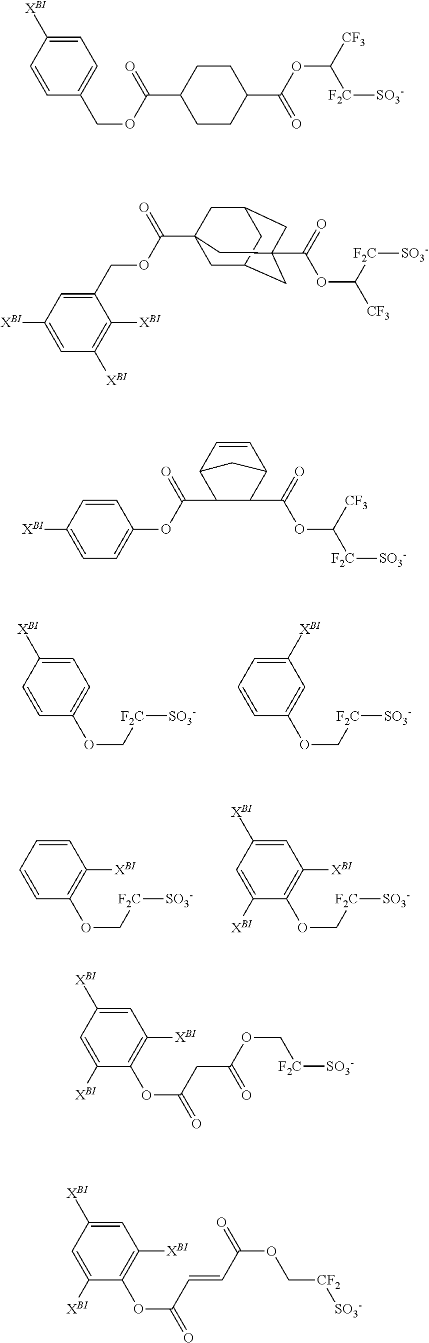

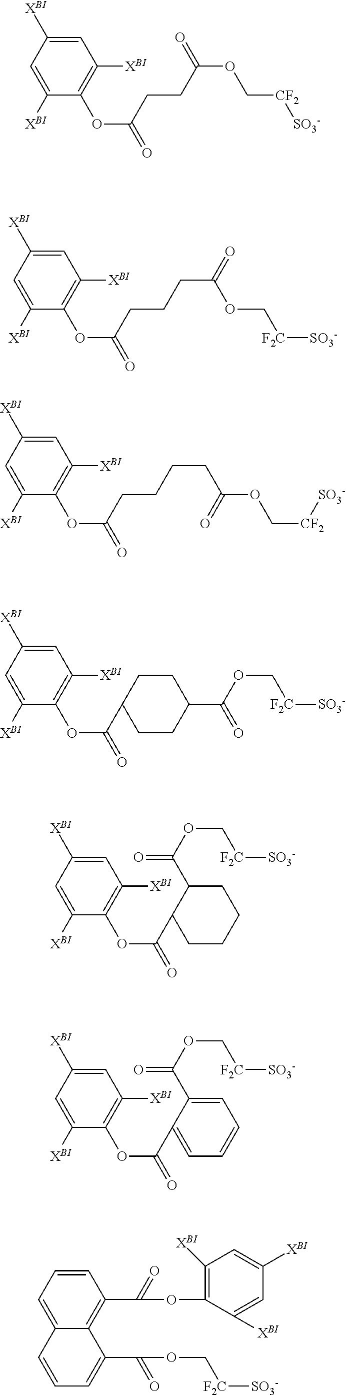

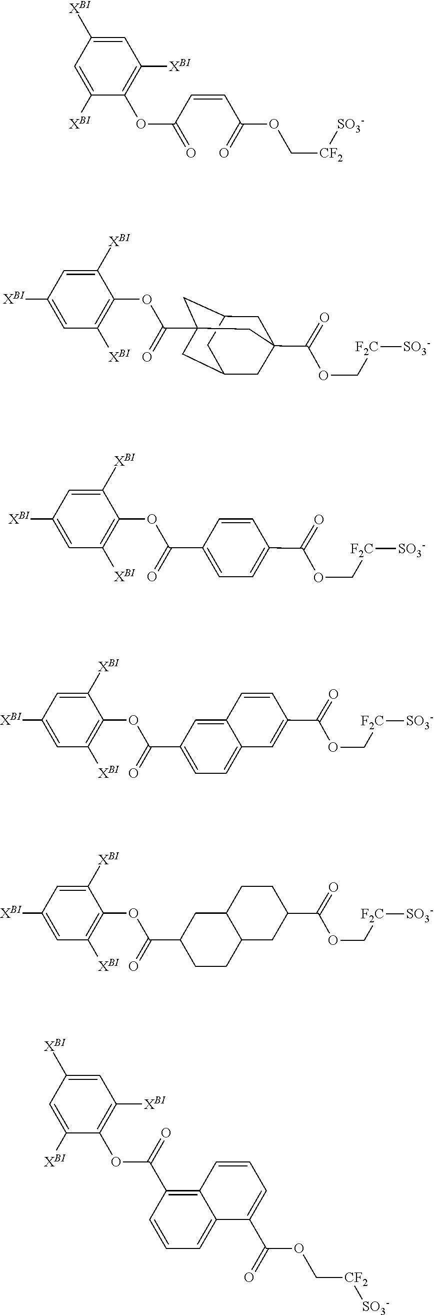

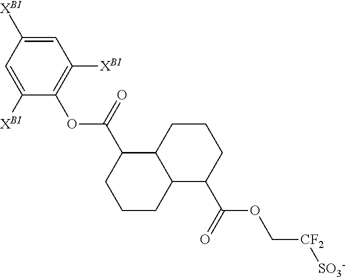

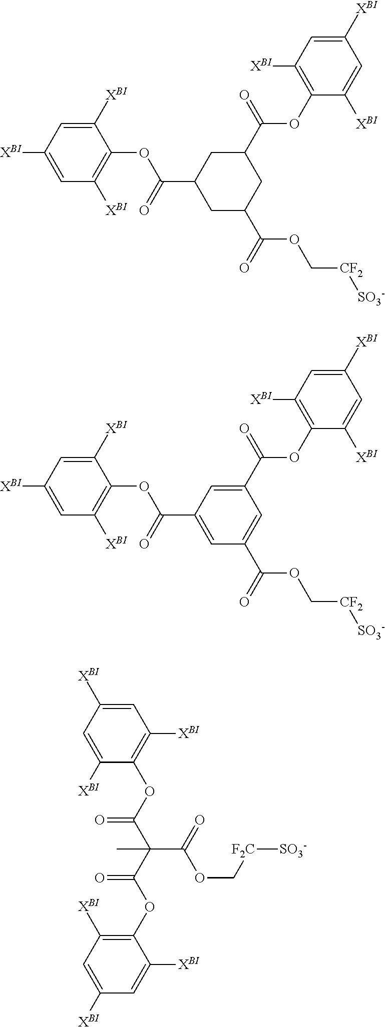

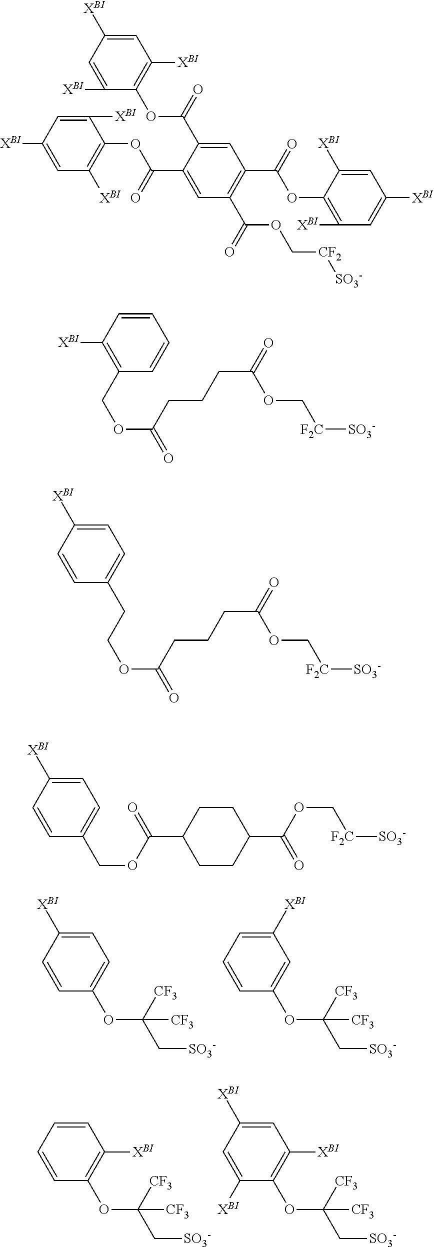

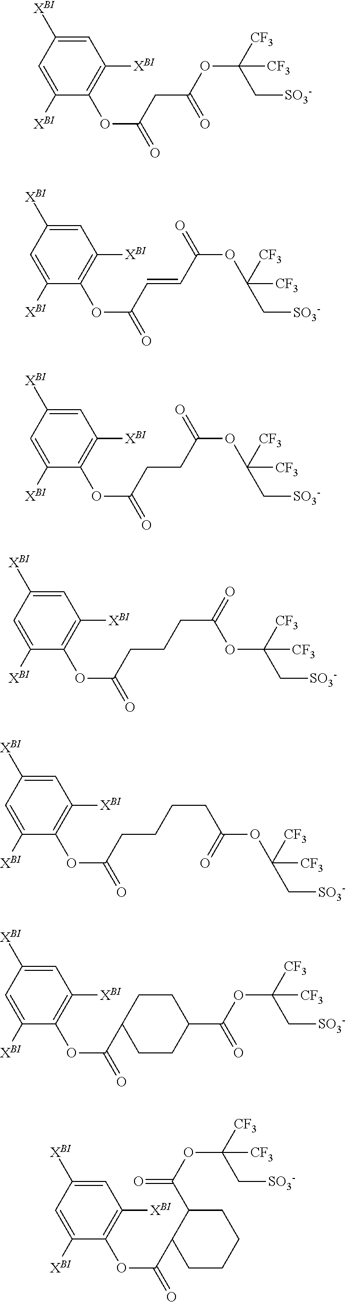

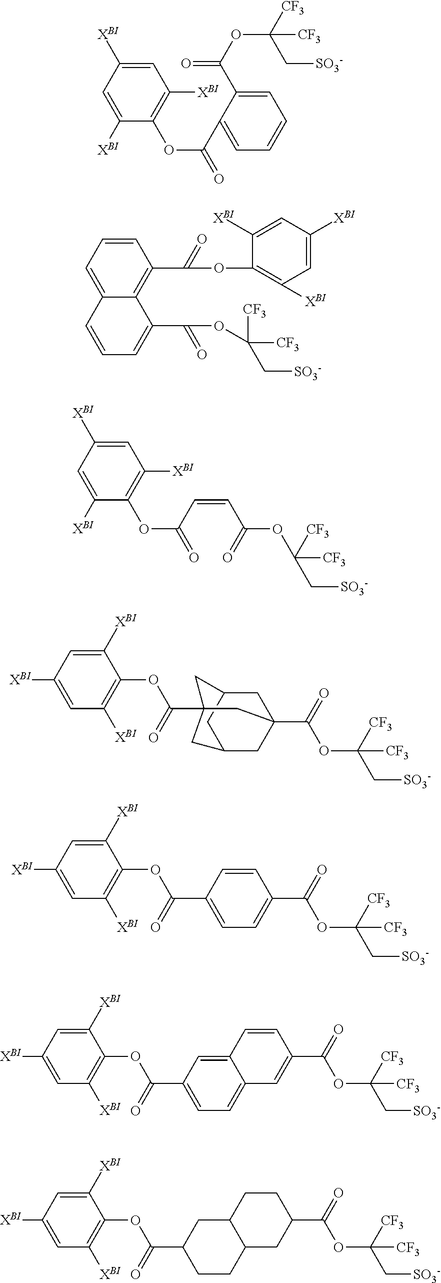

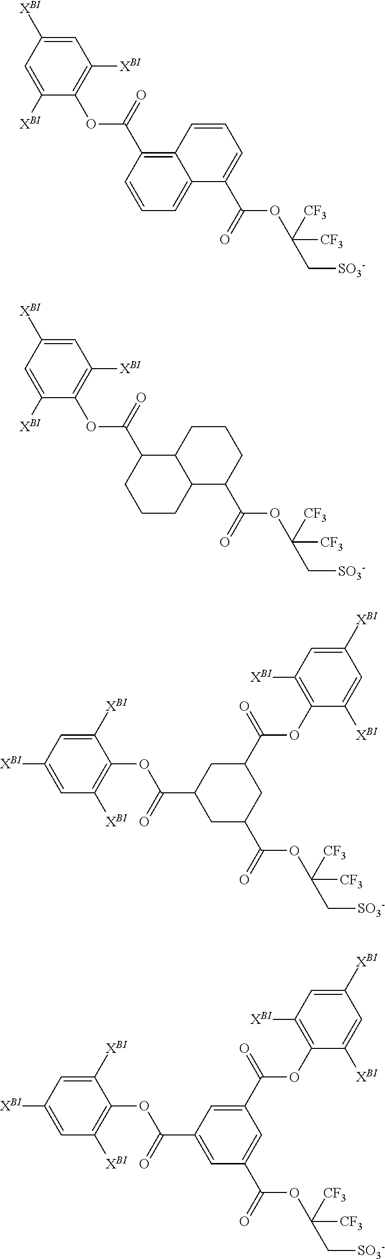

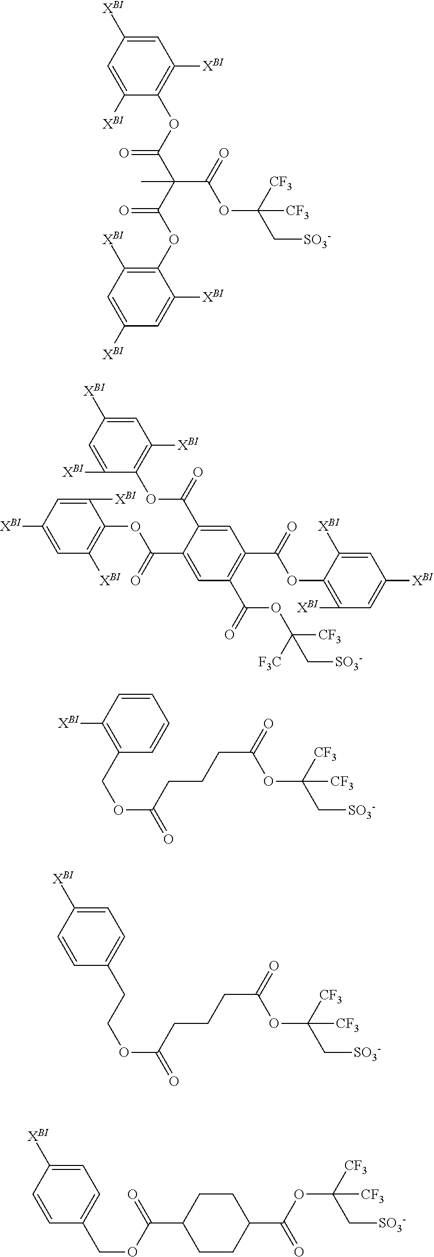

As the non-nucleophilic counter ion, a fluorobenzenesulfonic acid anion bonded to an aromatic group containing an iodine atom described in JP 6648726, an anion having a mechanism of decomposition by an acid described in WO 2021/200056 or JP-A 2021-70692, an anion having a cyclic ether group described in JP-A 2018-180525 or JP-A 2021-35935, and an anion described in JP-A 2018-92159 can also be used.

As the non-nucleophilic counter ion, a bulky benzenesulfonic acid derivative anion free of a fluorine atom described in JP-A 2006-276759, JP-A 2015-117200, JP-A 2016-65016, and JP-A 2019-202974, and a benzenesulfonic acid anion or an alkylsulfonic acid anion free of a fluorine atom bonded to an aromatic group containing an iodine atom described in JP 6645464 can also be used.

As the non-nucleophilic counter ion, an anion of a bissulfonic acid described in JP-A 2015-206932, an anion of a sulfonamide or a sulfonimide different from a sulfonic acid on one side and an anion of a carboxylic acid on the other side described in WO 2020/158366, and an anion of a sulfonic acid on one side and an anion of a carboxylic acid on the other side described in JP-A 2015-24989 can be further used.

In formulae (c2) and (c3), d1 and d2 are each independently 0, 1, 2, or 3, and preferably 1.

In formula (c4), e1 is 0 or 1. e2 is 0, 1, 2, 3, or 4. e3 is 0, 1, 2, 3, or 4. Provided that 0≤e2+e3≤4 when e1 is 0, and 0≤e2+e3≤6 when e1 is 1.

In the formulae (c2), (c3), and (c4), L¹ is a single bond, an ether bond, an ester bond, a carbonyl group, a sulfonate ester bond, a sulfonamide bond, a carbonate bond, or a carbamate bond. Inter alia, from the viewpoint of synthesis, an ether bond, an ester bond, and a carbonyl group are preferred, and an ester bond and a carbonyl group are more preferred.

In formula (c2), R^{f1} and R^{f2} are each independently a fluorine atom, or a C₁-C₆ fluorinated saturated hydrocarbyl group. Inter alia, R^{f1} and R^{f2} are each preferably a fluorine atom in order to increase the acid strength of the generated acid. R^{f3} and Rf⁴ are each independently a hydrogen atom, a fluorine atom, or a C₁-C₆ fluorinated saturated hydrocarbyl group. Inter alia, at least one of R^{f3} and R^{f4} is preferably a trifluoromethyl group for improving solvent solubility.

In formula (c3), R^{f5} and R^{f6} are each independently a hydrogen atom, a fluorine atom, or a C₁-C₆ fluorinated saturated hydrocarbyl group. Provided that not all R^{f5} and R^{f6} are simultaneously hydrogen atoms. Inter alia, at least one of R^{f5} and R^{f6} is preferably a trifluoromethyl group for improving solvent solubility.

In formula (c4), Rf⁷ is a fluorine atom, a C₁-C₆ fluorinated alkyl group, a C₁-C₆ fluorinated alkoxy group, or a C₁-C₆ fluorinated alkylthio group. As Rf⁷, a fluorine atom, a trifluoromethyl group, a difluoromethyl group, a trifluoromethoxy group, a difluoromethoxy group, a trifluoromethylthio group, or a difluoromethylthio group is preferred, and a fluorine atom, a trifluoromethyl group, or a trifluoromethoxy group is more preferred. When f is 2, 3, or 4, Rf⁷ may be identical or different.

In formula (c4), R³³ is a halogen atom other than a fluorine atom or a C₁-C₂₀ hydrocarbyl group which may contain a heteroatom. The hydrocarbyl group may be saturated or unsaturated and straight, branched, or cyclic. Specific examples thereof are as exemplified above for the hydrocarbyl group of R¹ in the description of formula (1), but not limited thereto. When e3 is 2, 3, or 4, R³³ may be identical or different.

When e3 is 2, 3, or 4, a plurality of R³³ may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. In the ring, some or all of the hydrogen atoms may be substituted by a moiety containing a heteroatom such as an oxygen atom, a sulfur atom, a nitrogen atom, or a halogen atom, and some constituent —CH₂—may be replaced by a moiety containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

Specific examples of the anion of the repeat unit c2 are shown below, but not limited thereto. Herein R^{A} is as defined above, and Me is a methyl group.

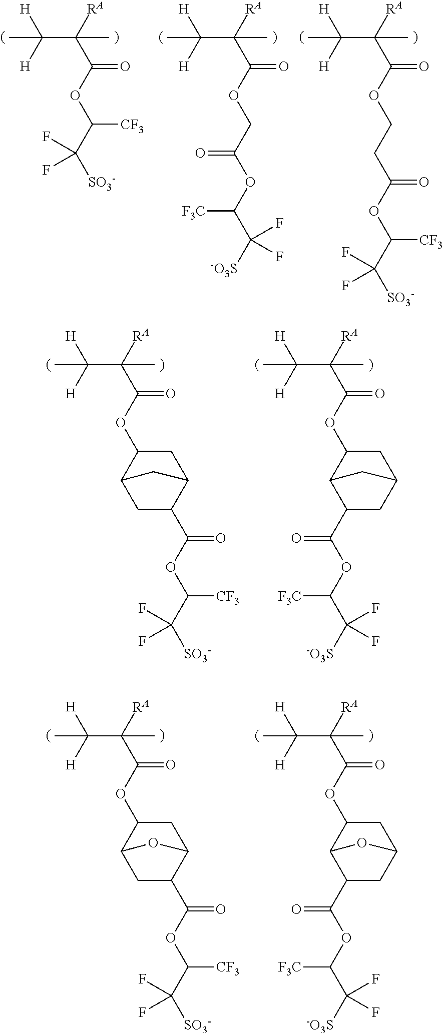

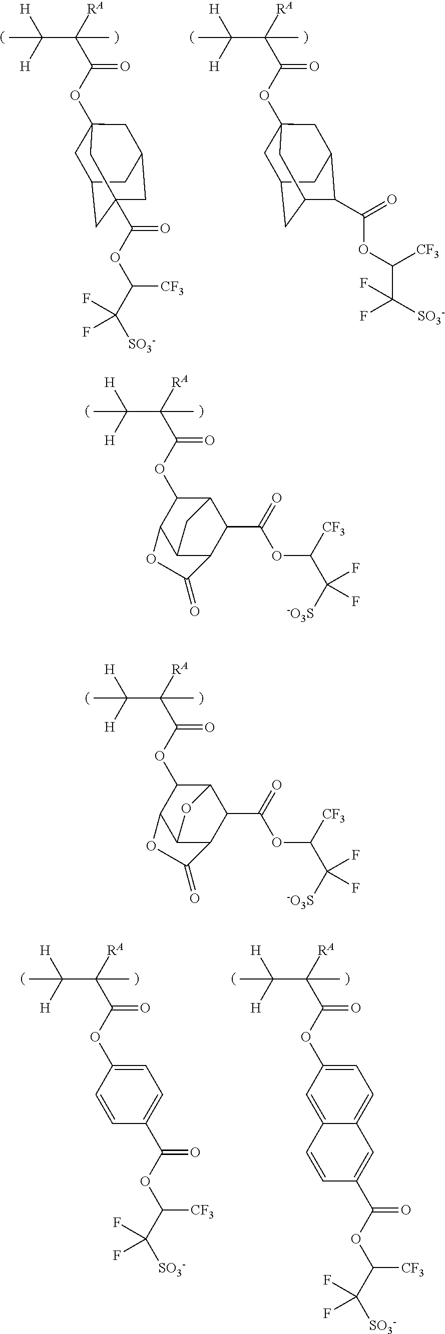

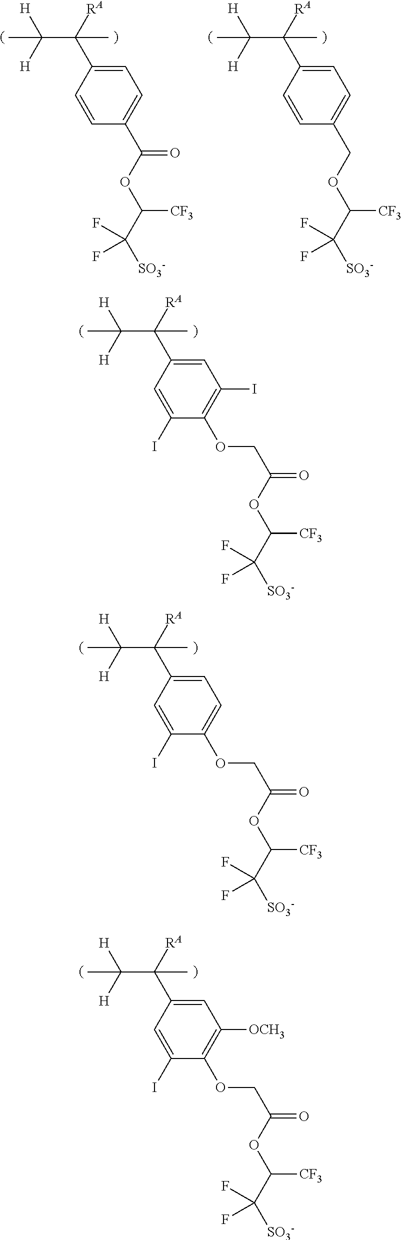

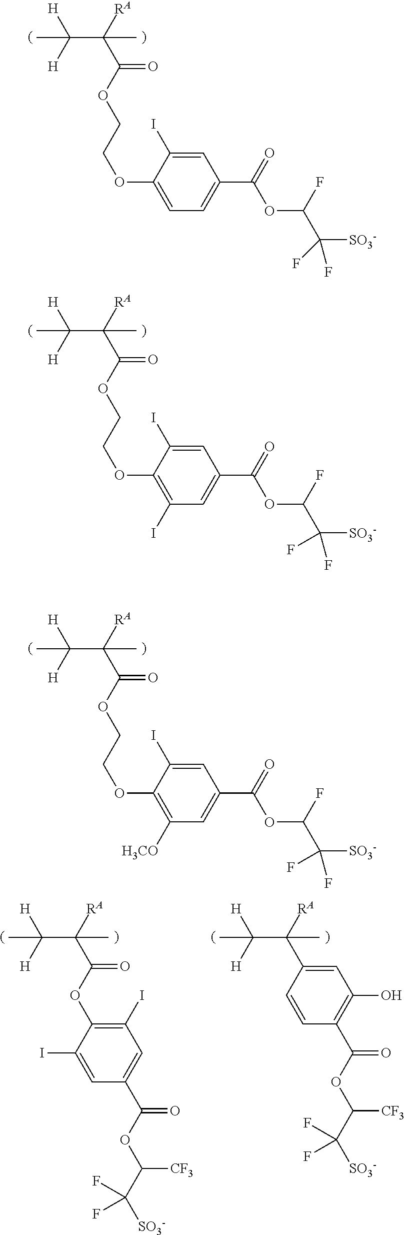

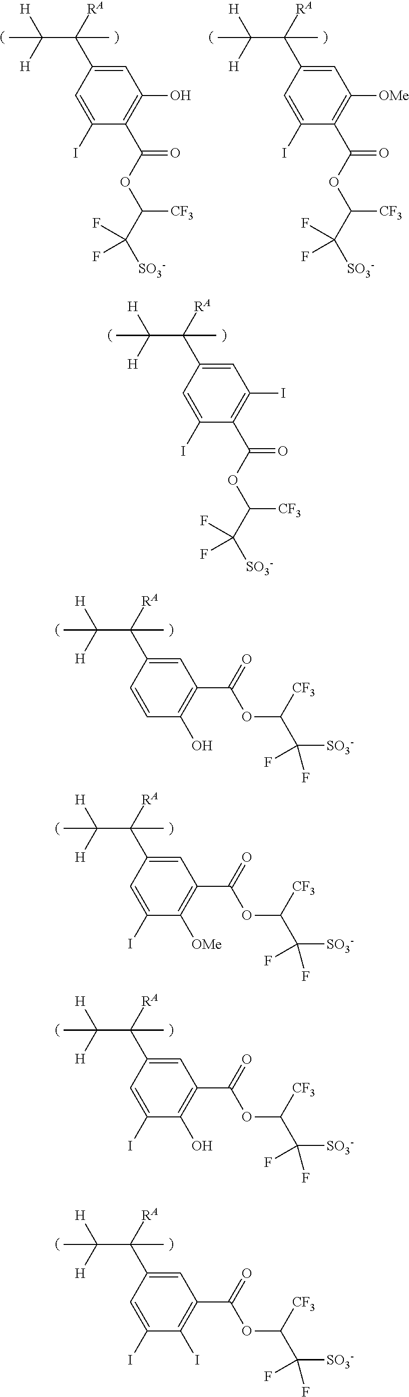

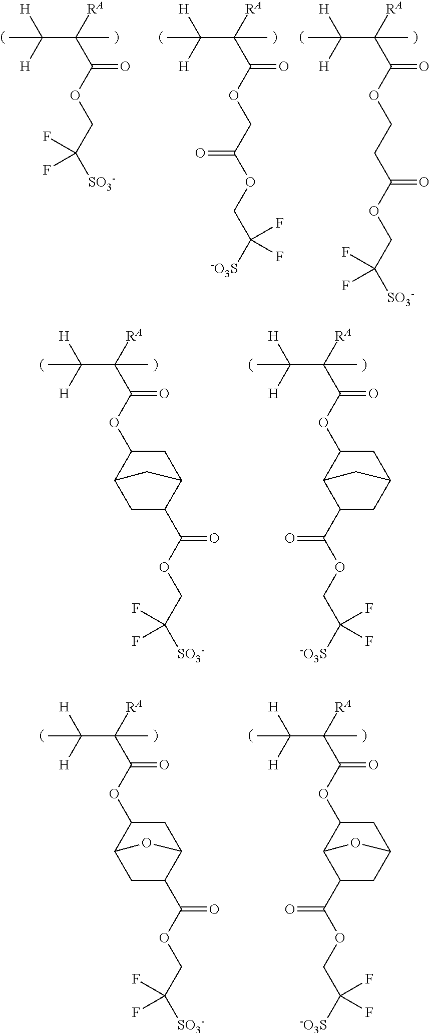

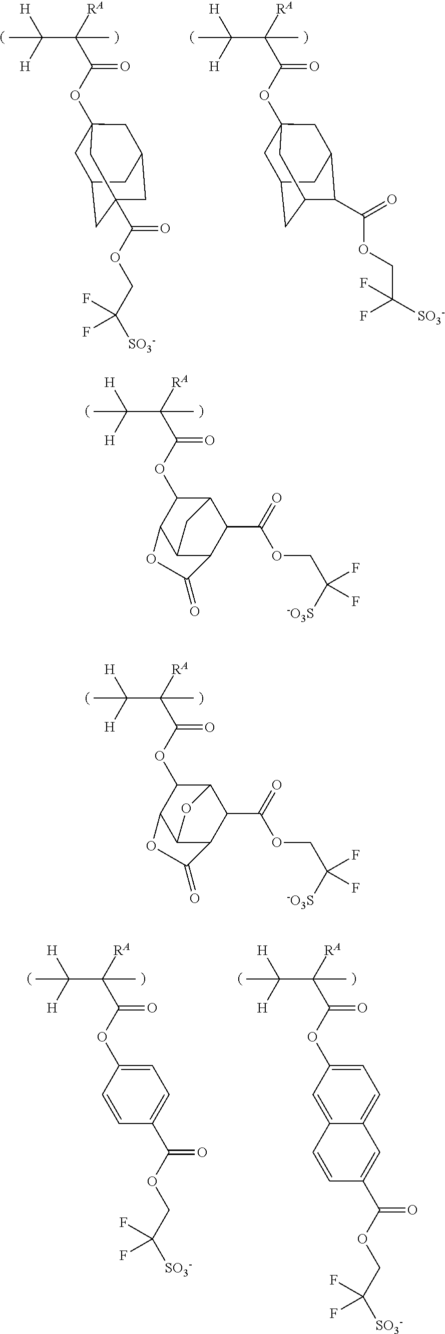

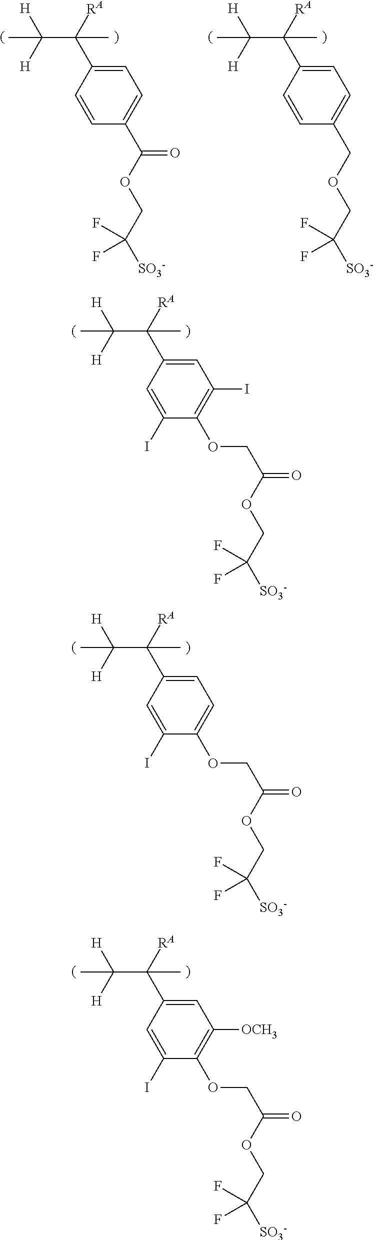

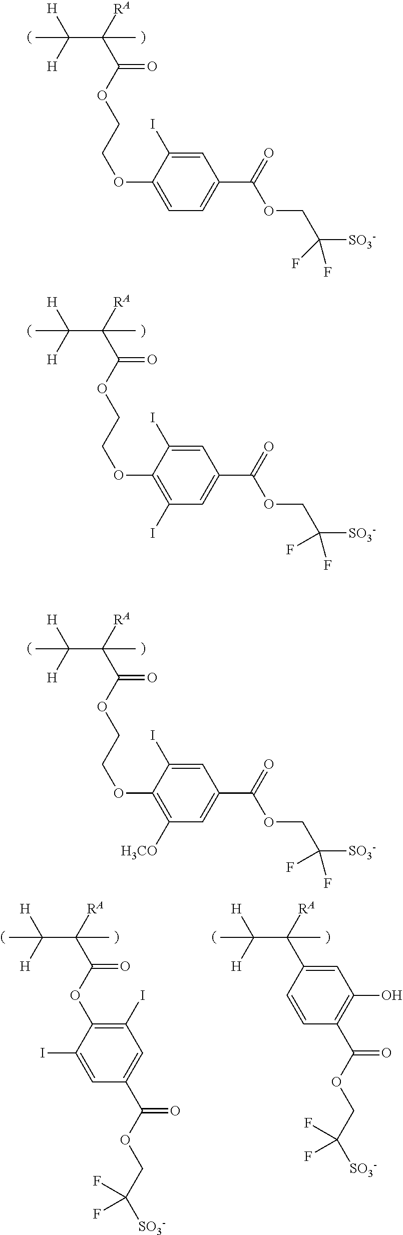

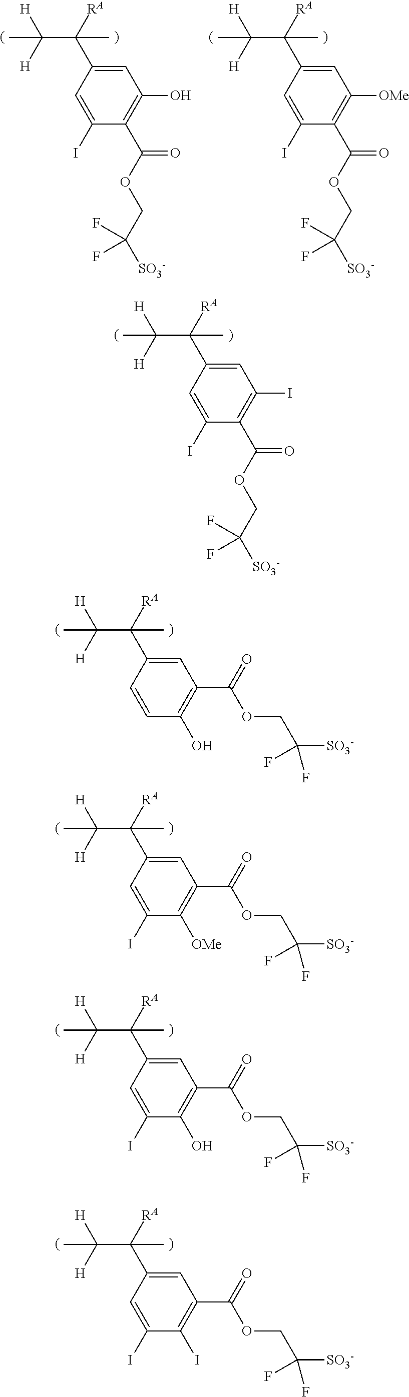

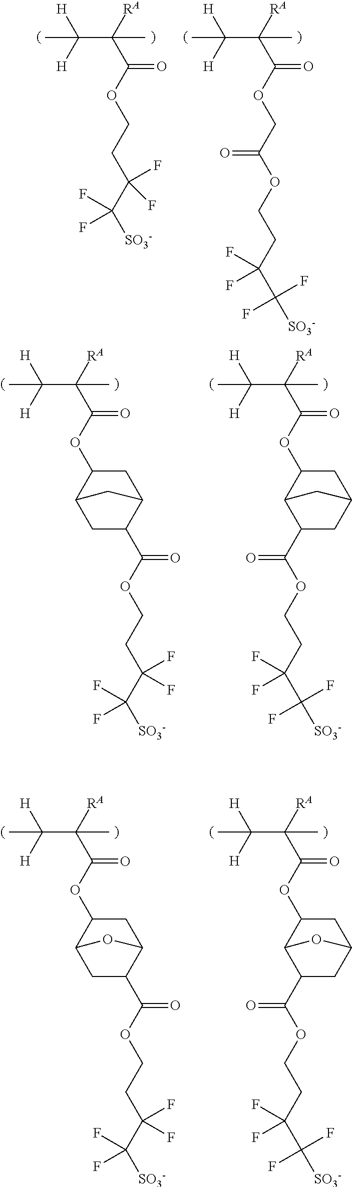

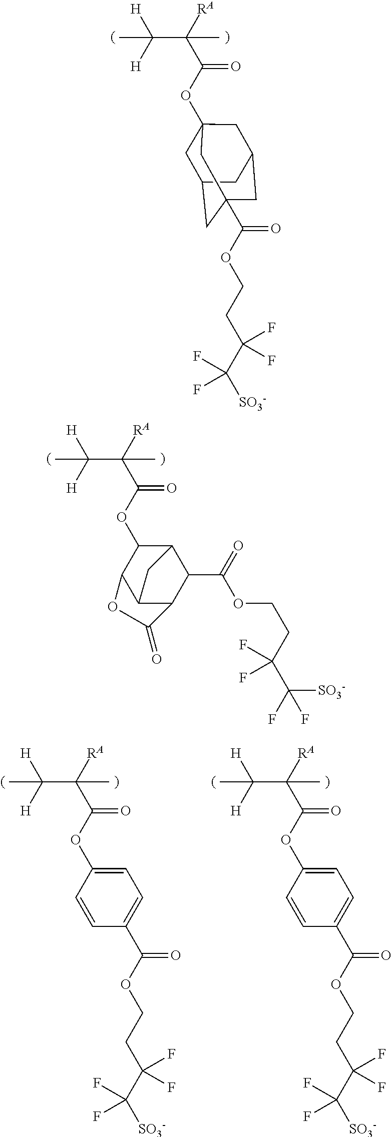

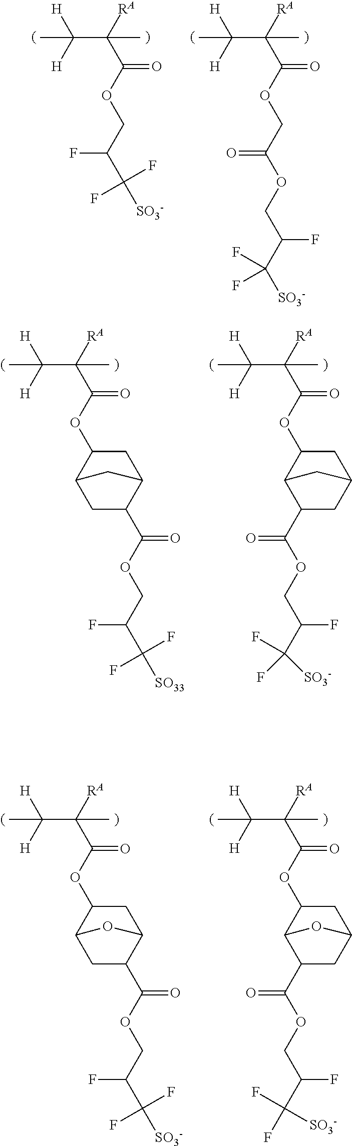

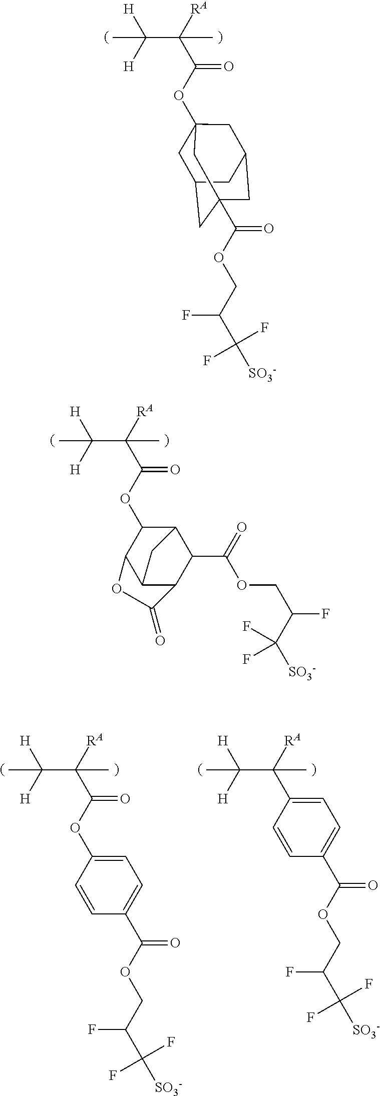

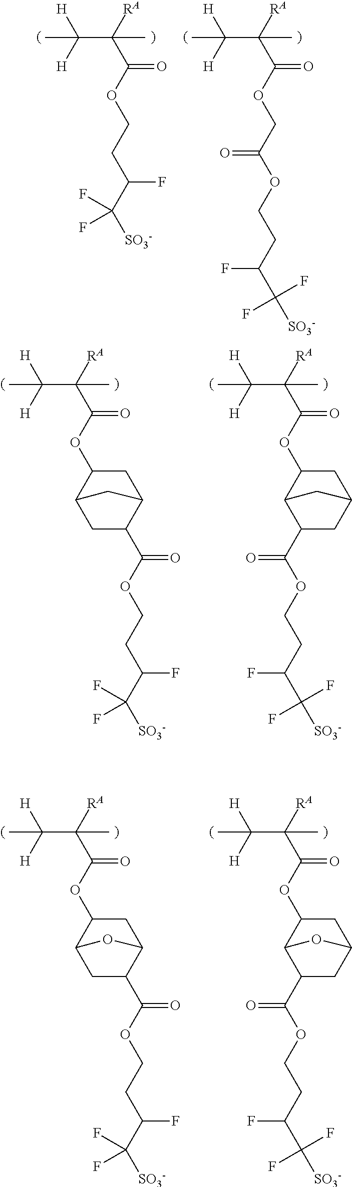

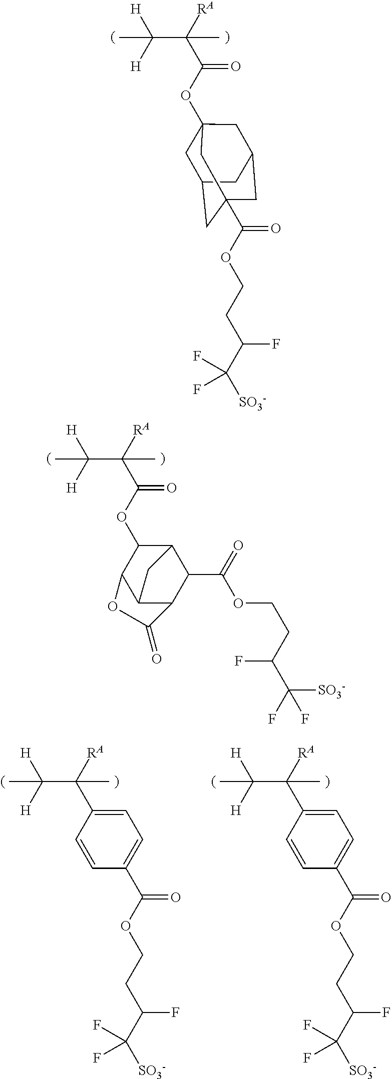

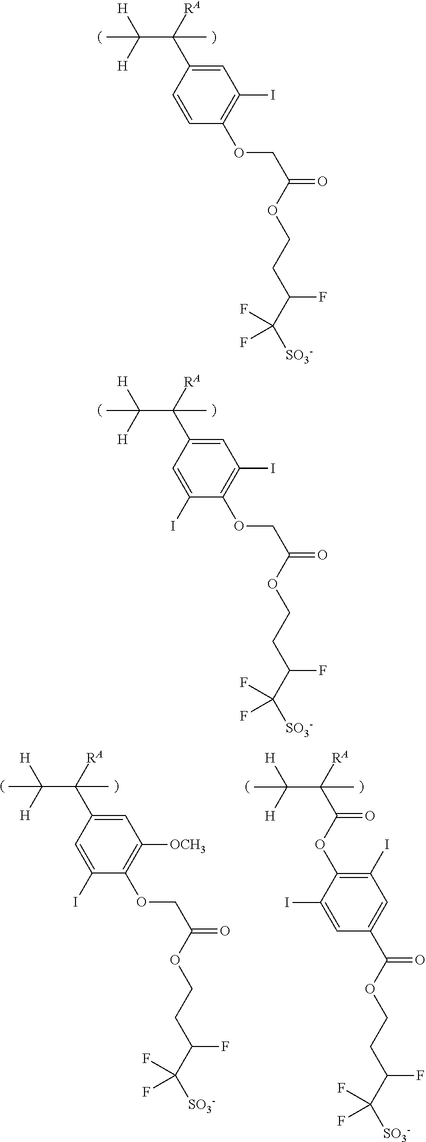

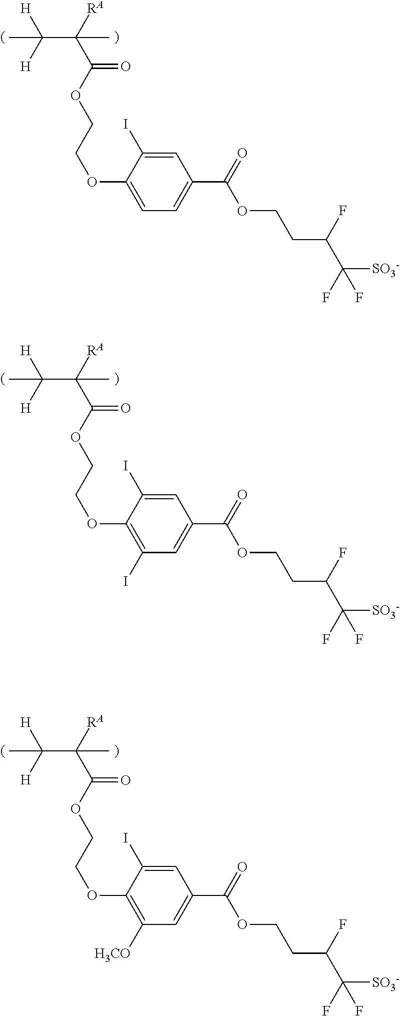

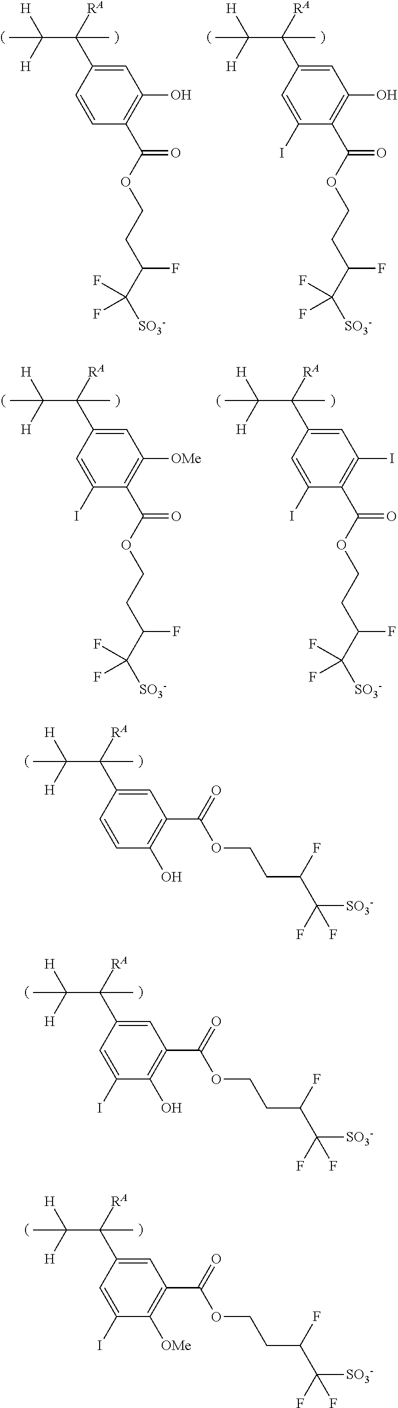

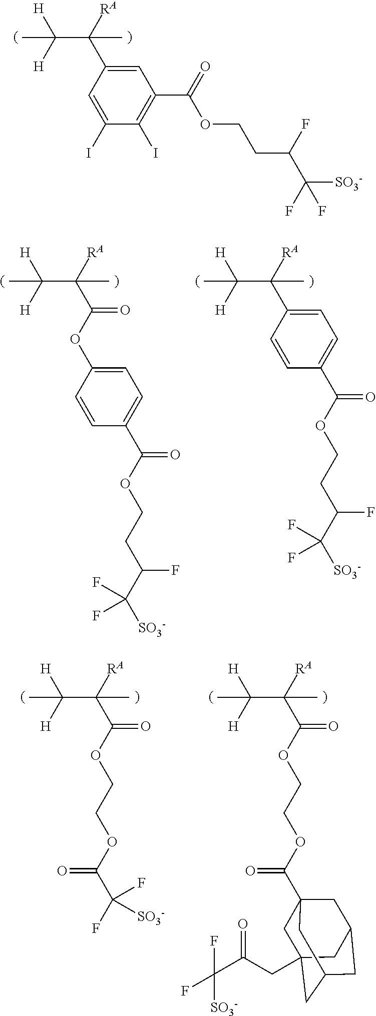

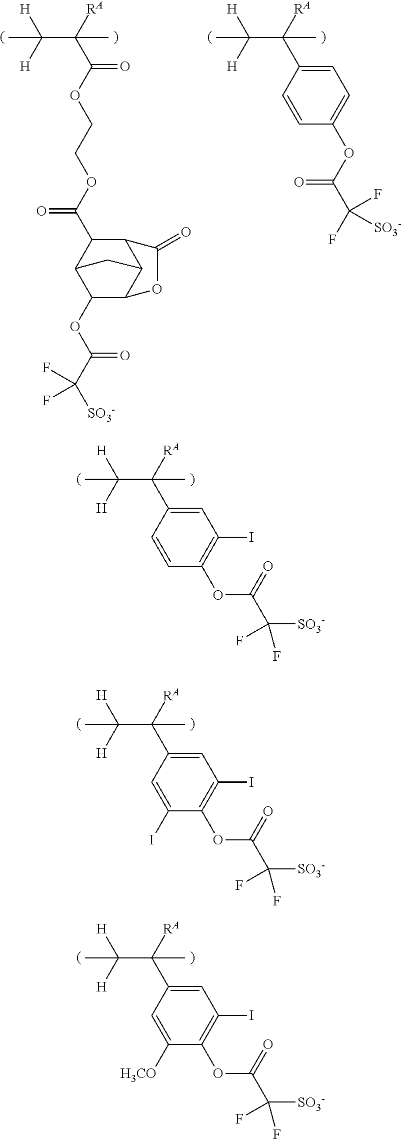

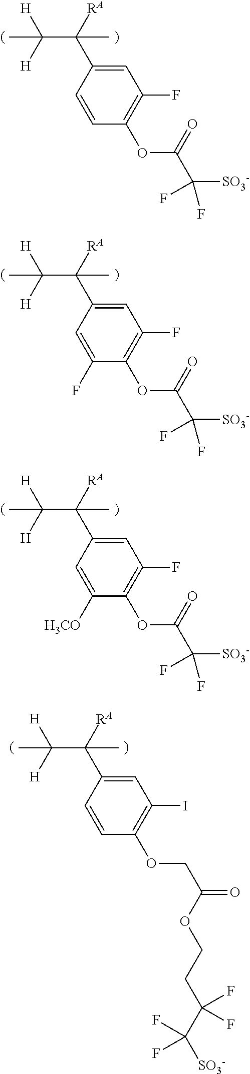

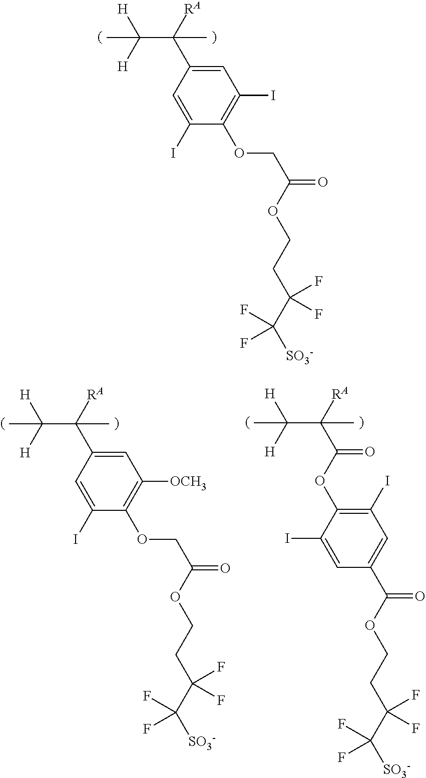

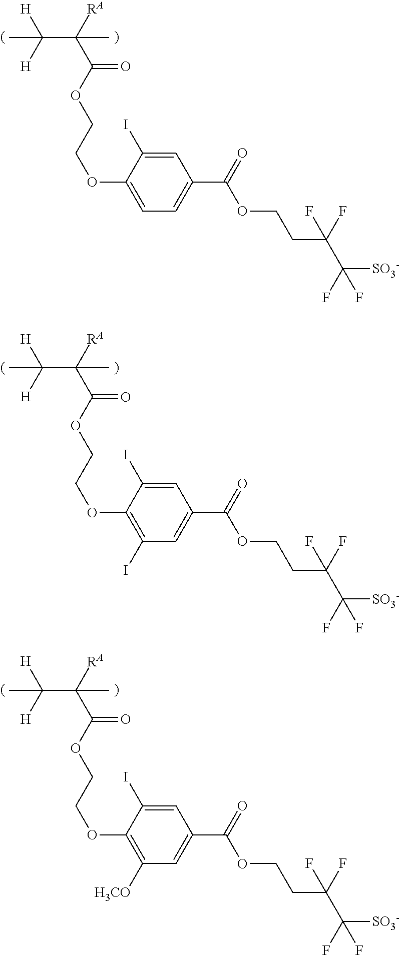

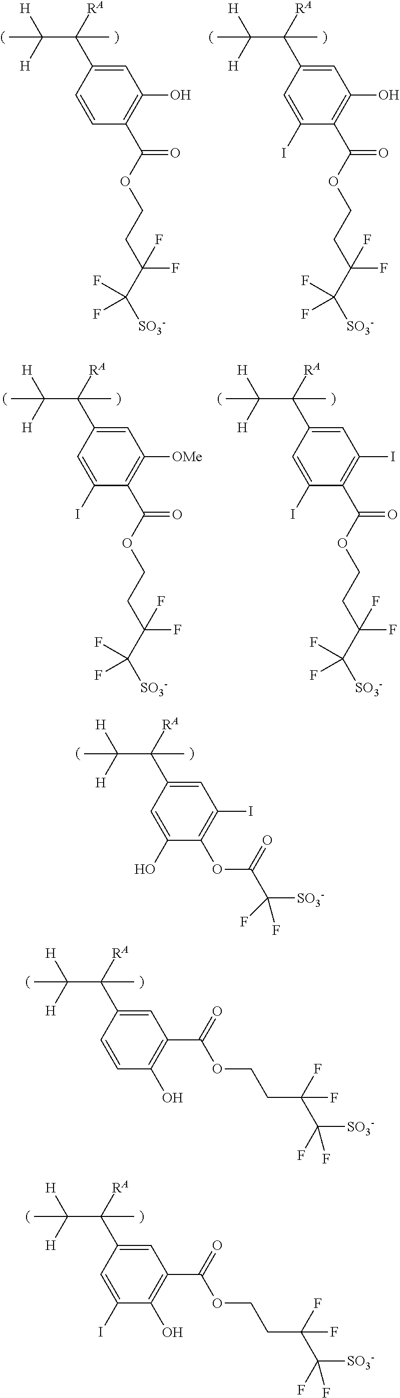

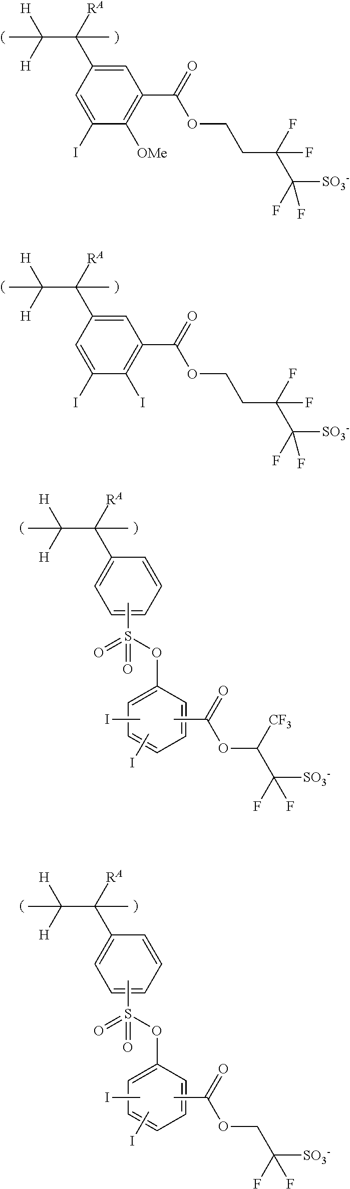

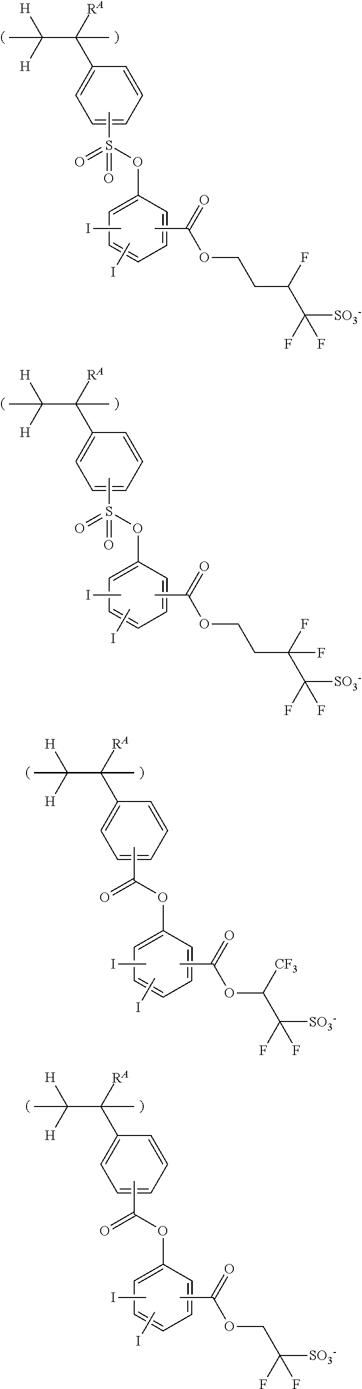

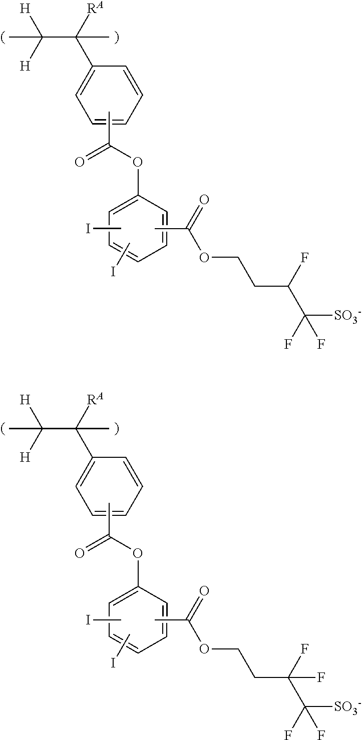

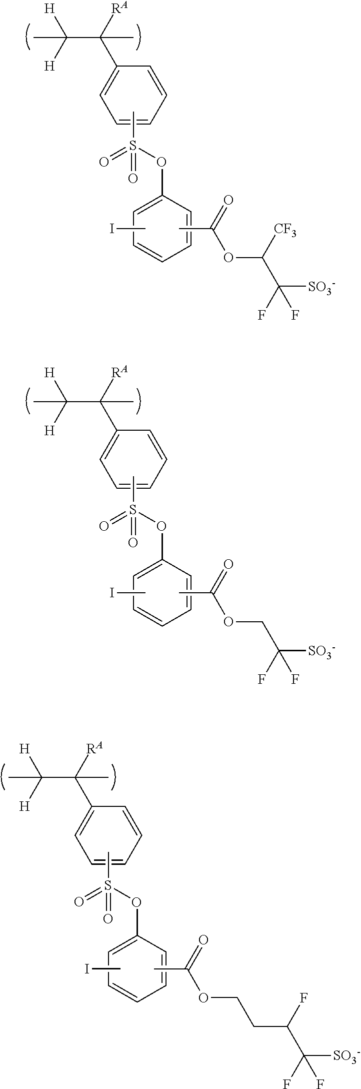

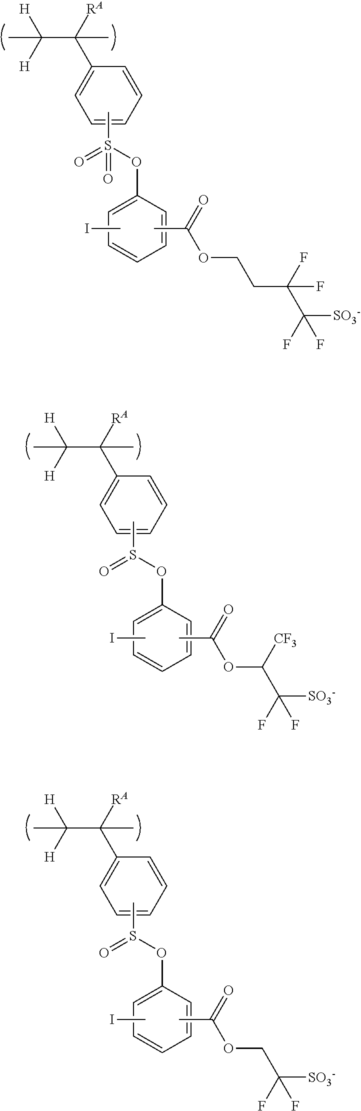

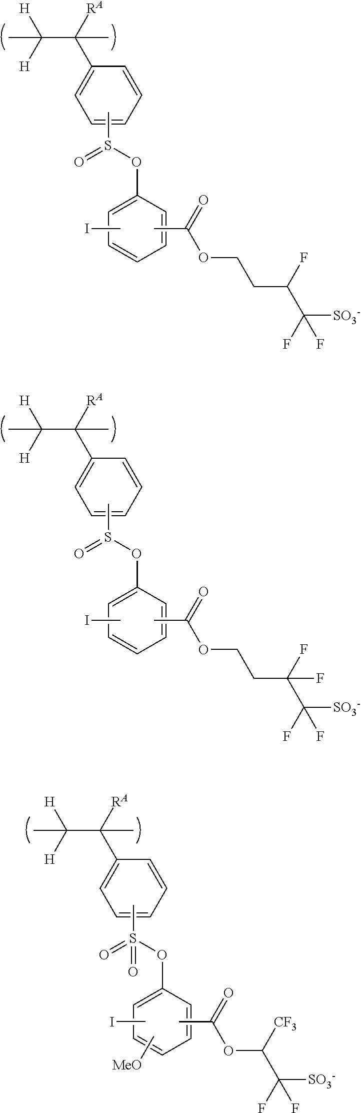

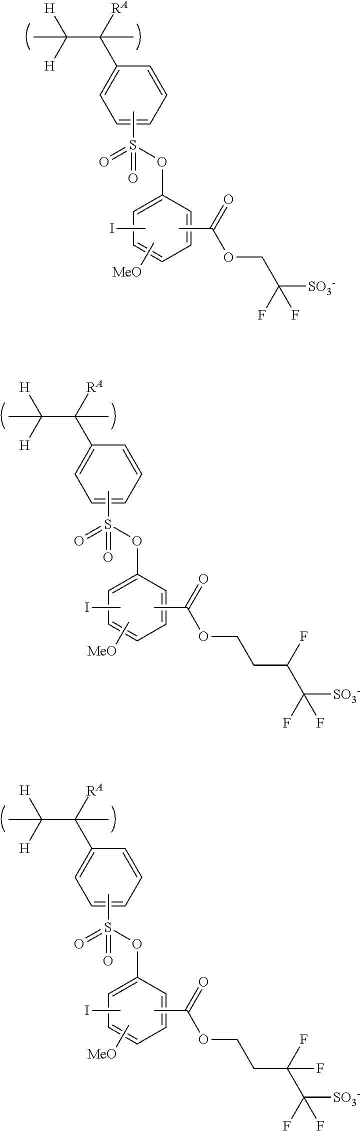

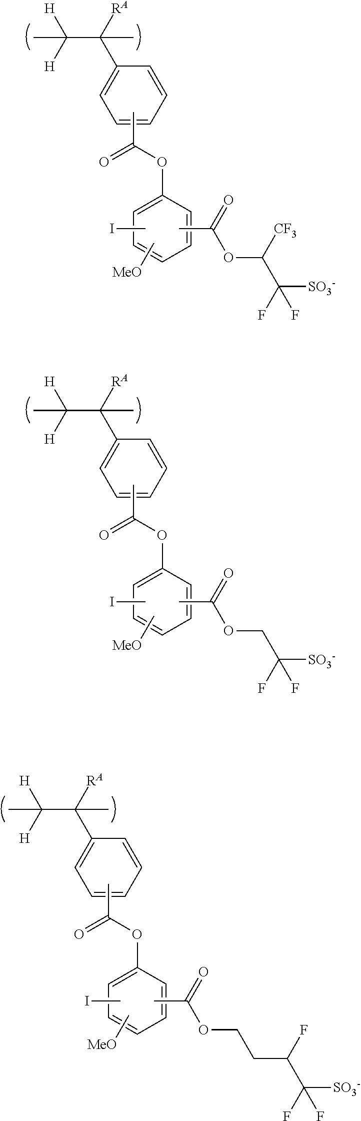

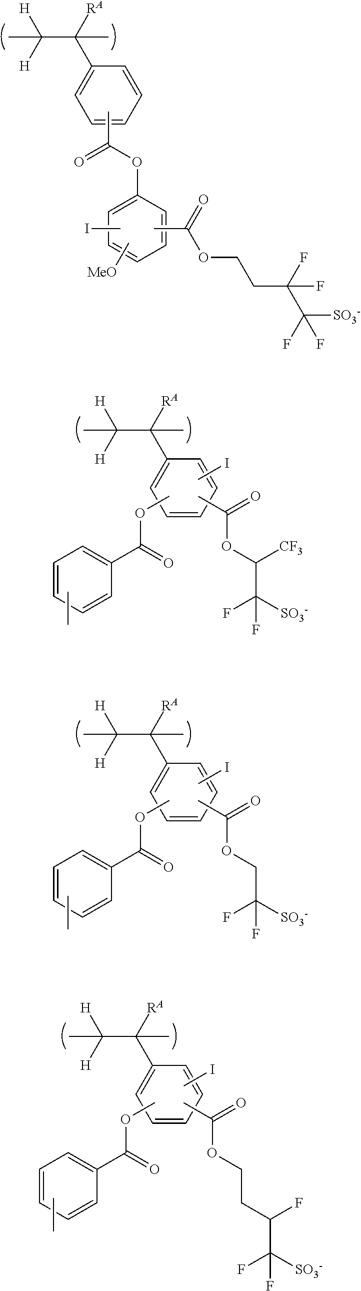

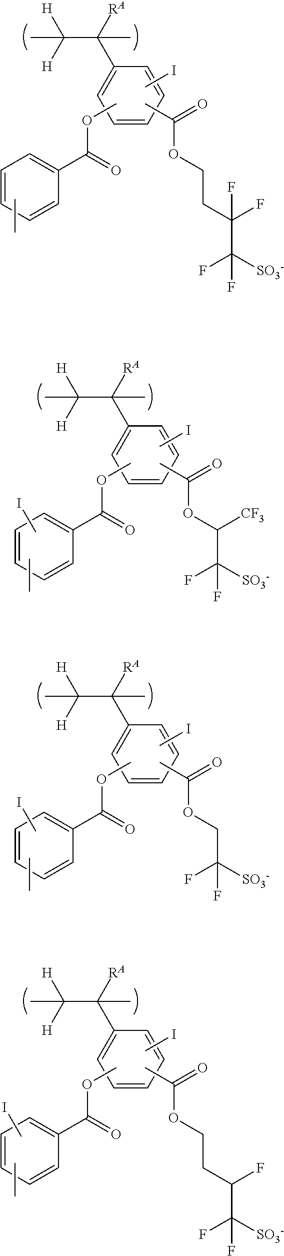

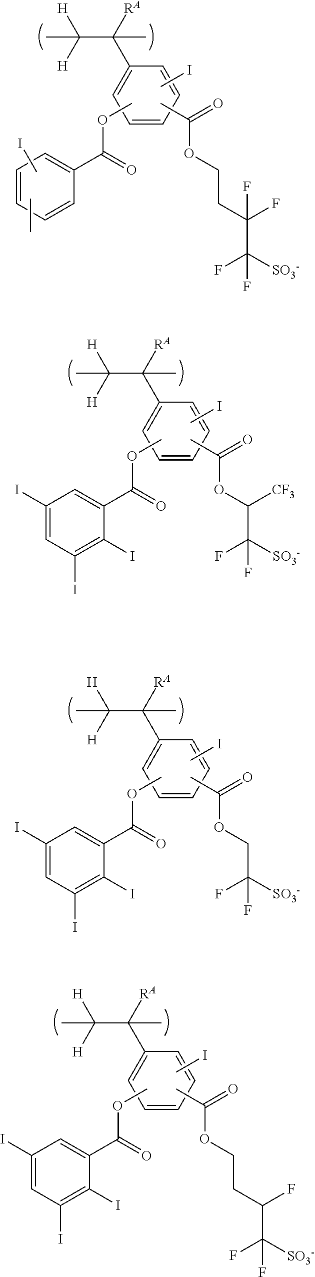

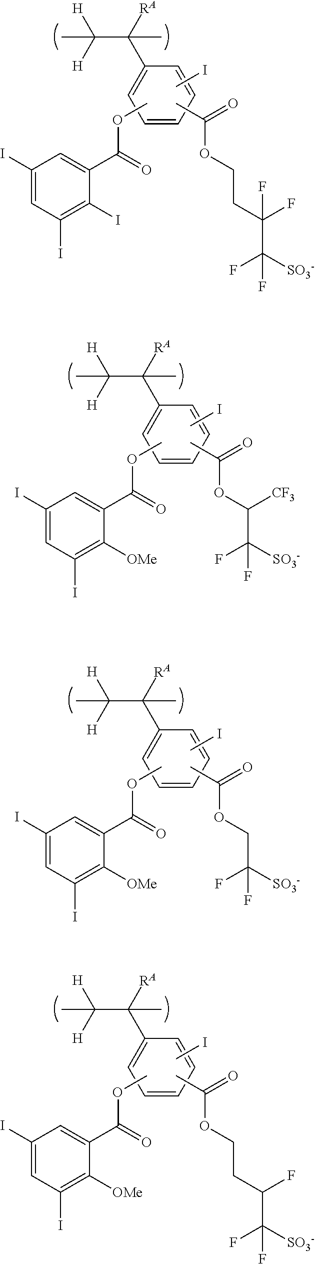

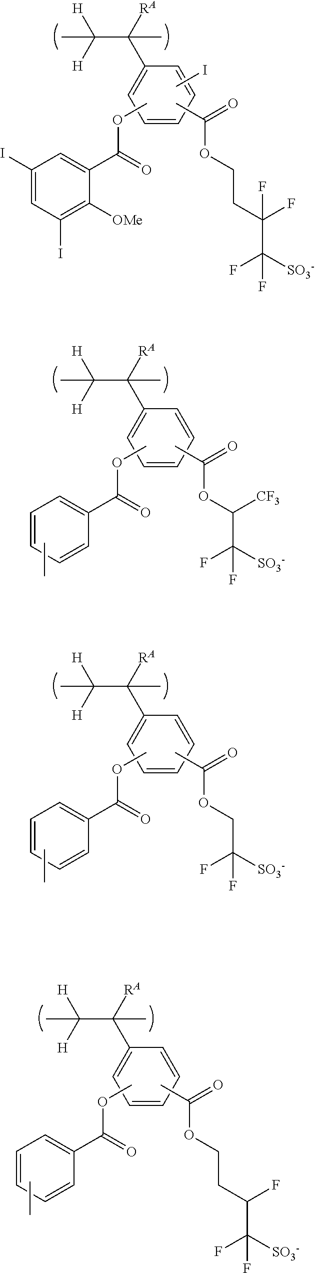

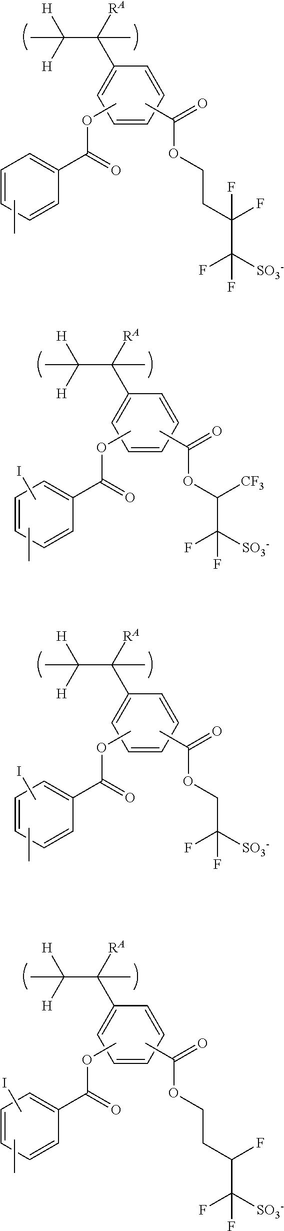

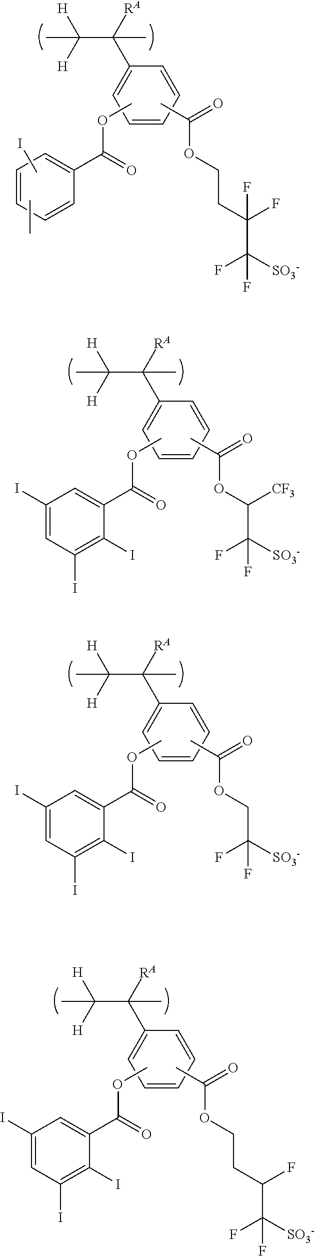

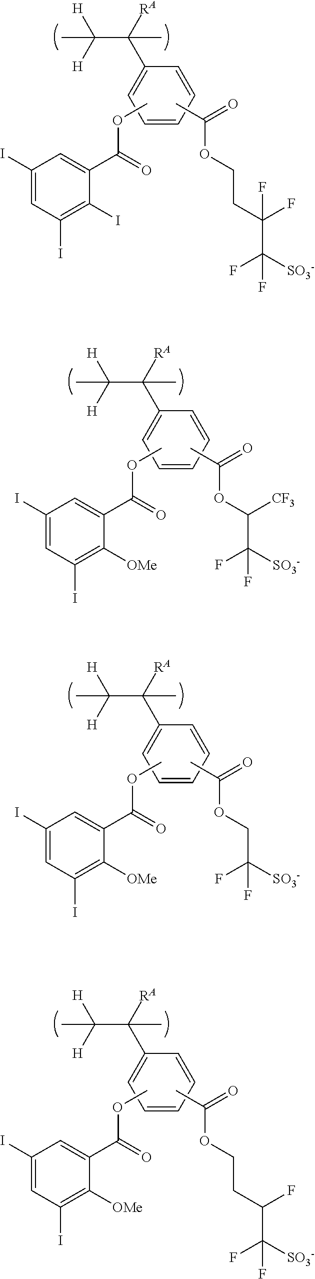

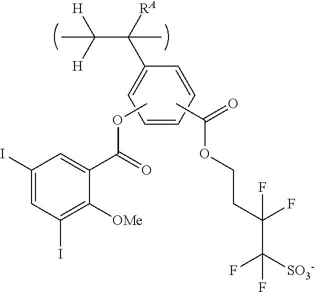

Specific examples of the anion of the repeat unit c3 are shown below, but not limited thereto. Herein R^{A} is as defined above.

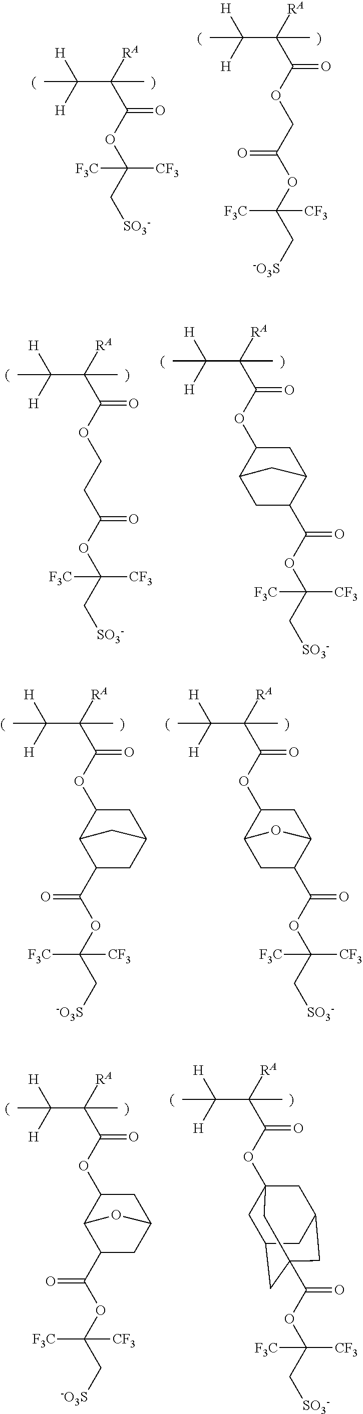

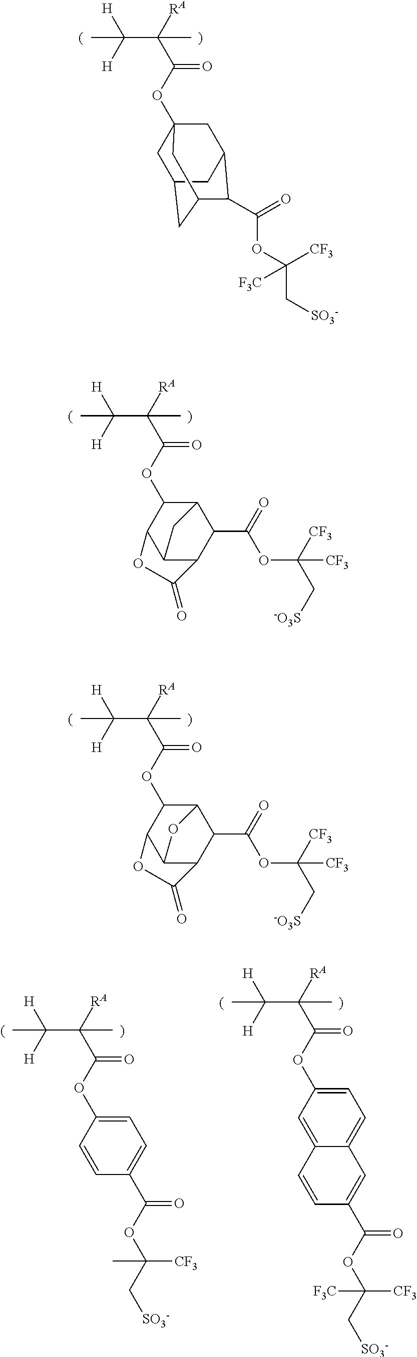

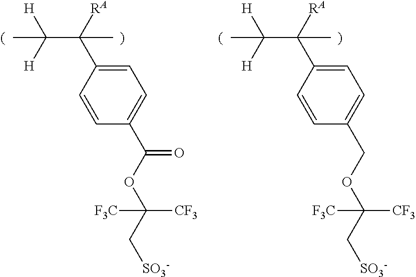

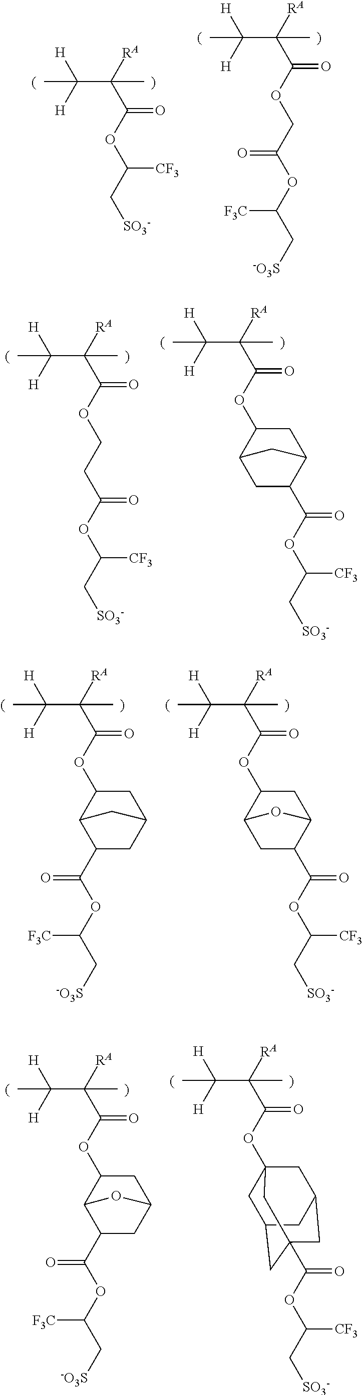

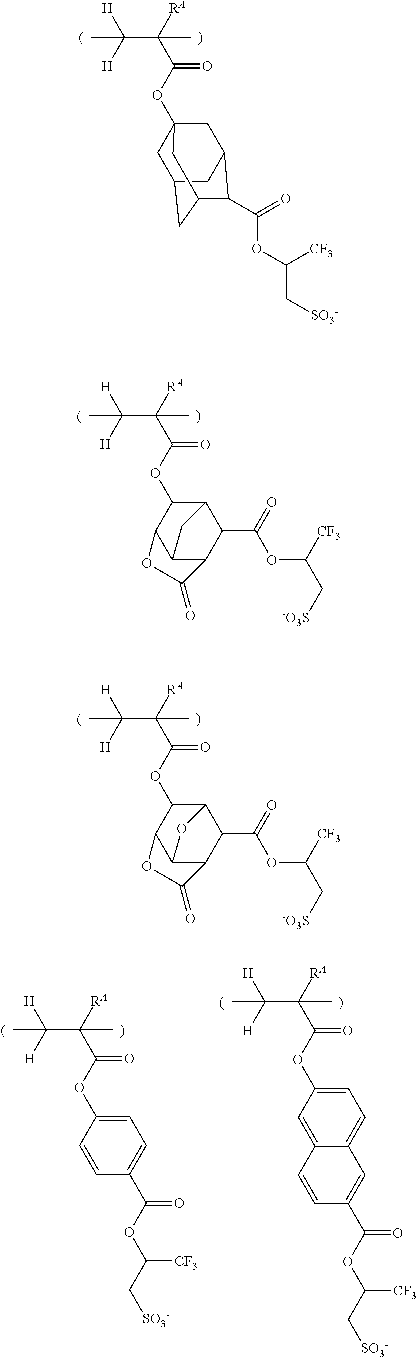

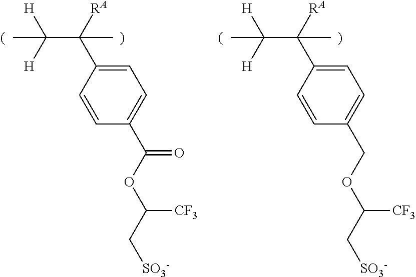

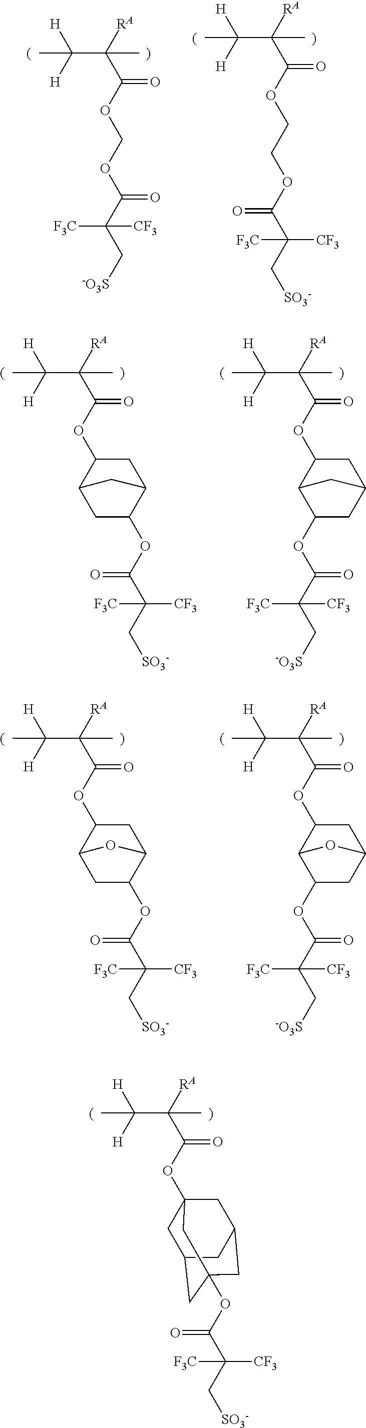

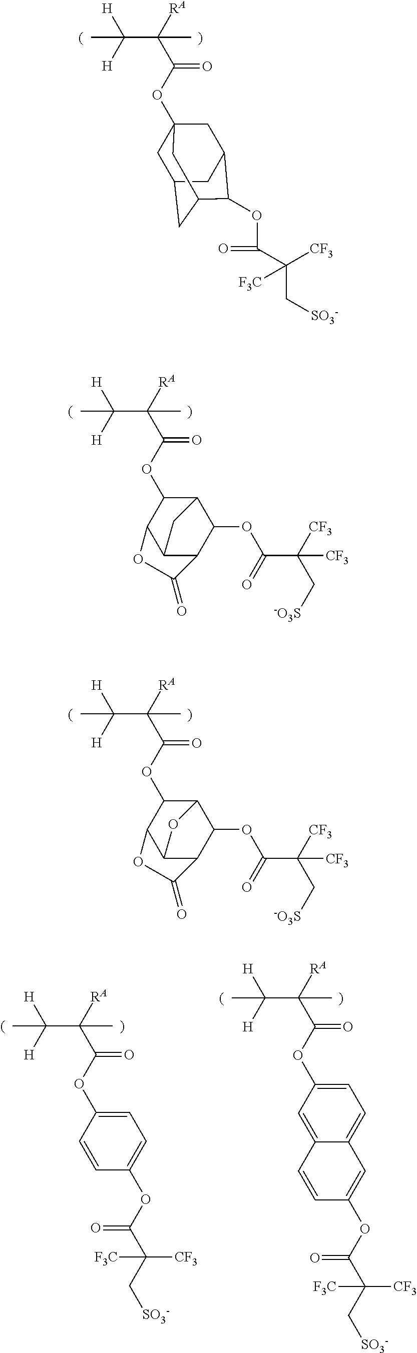

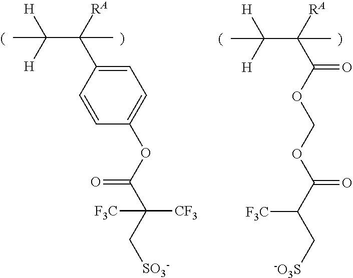

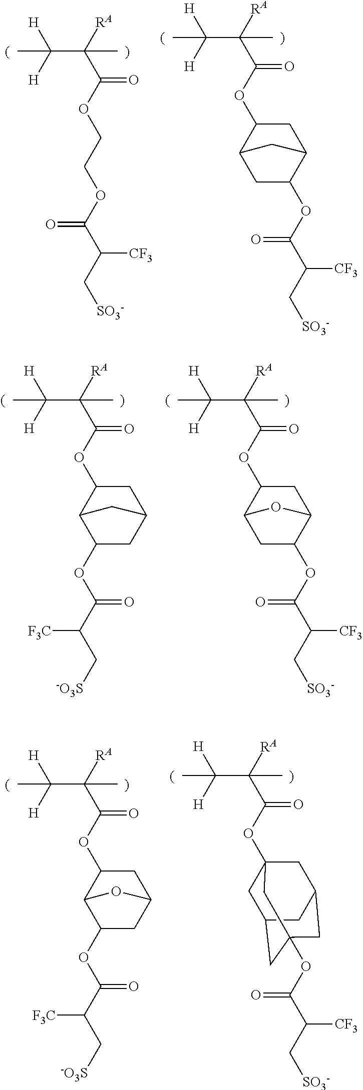

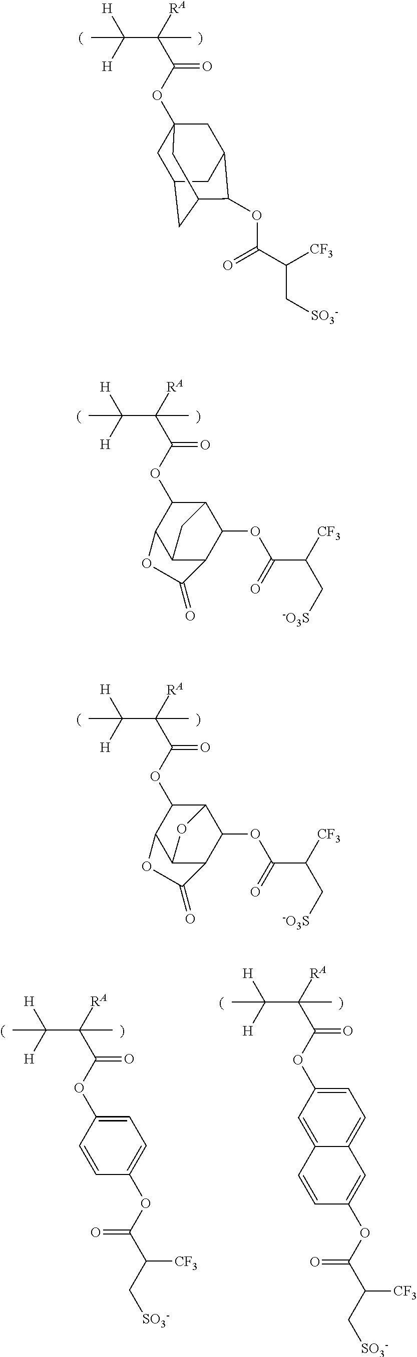

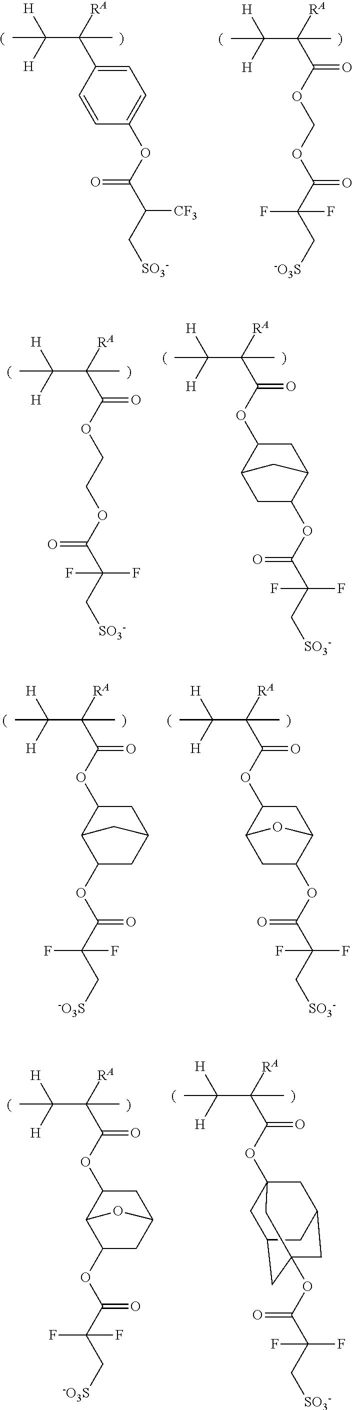

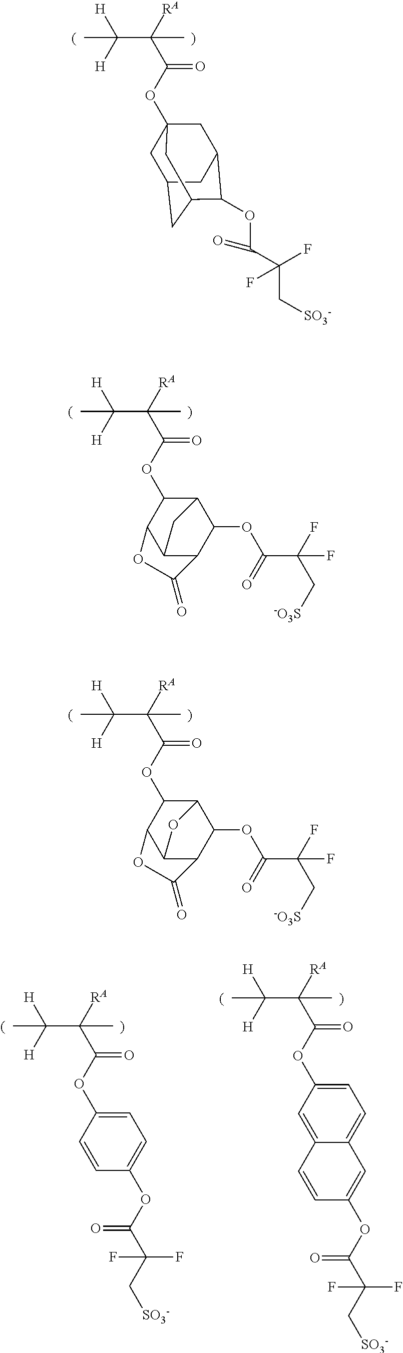

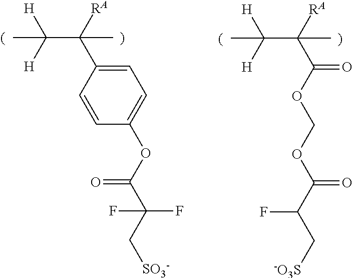

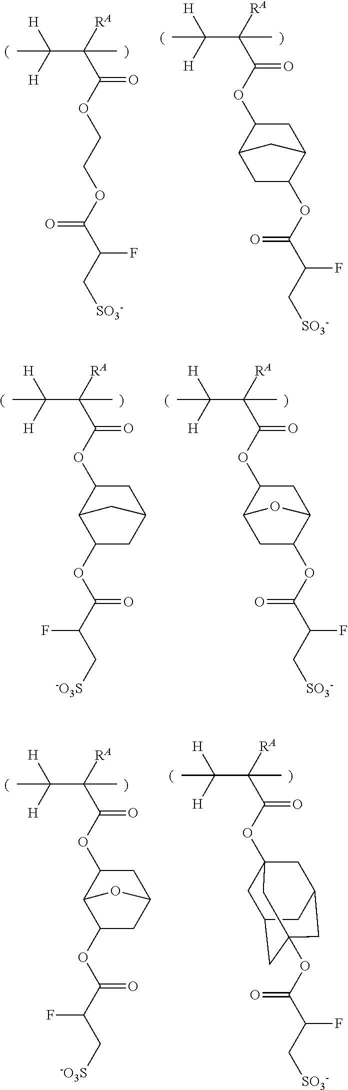

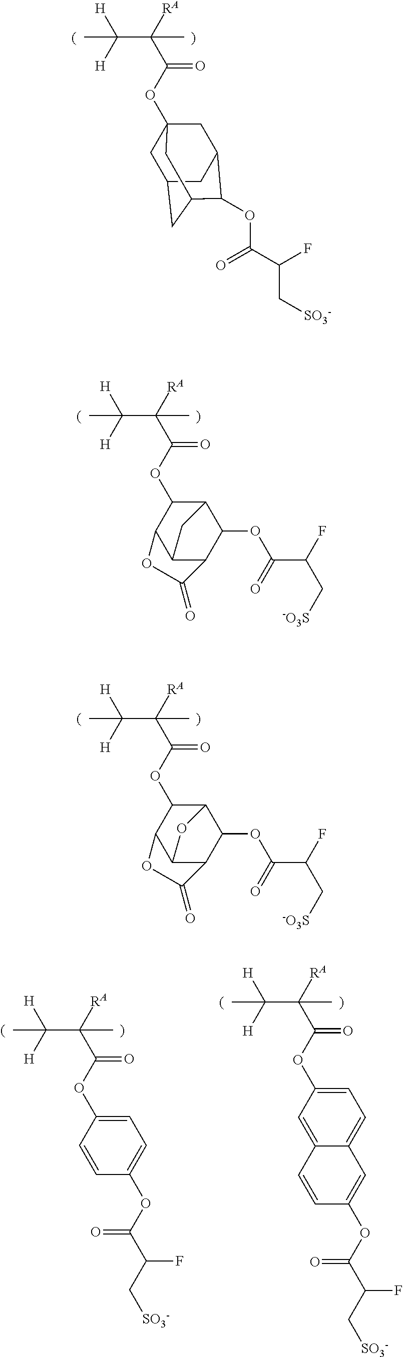

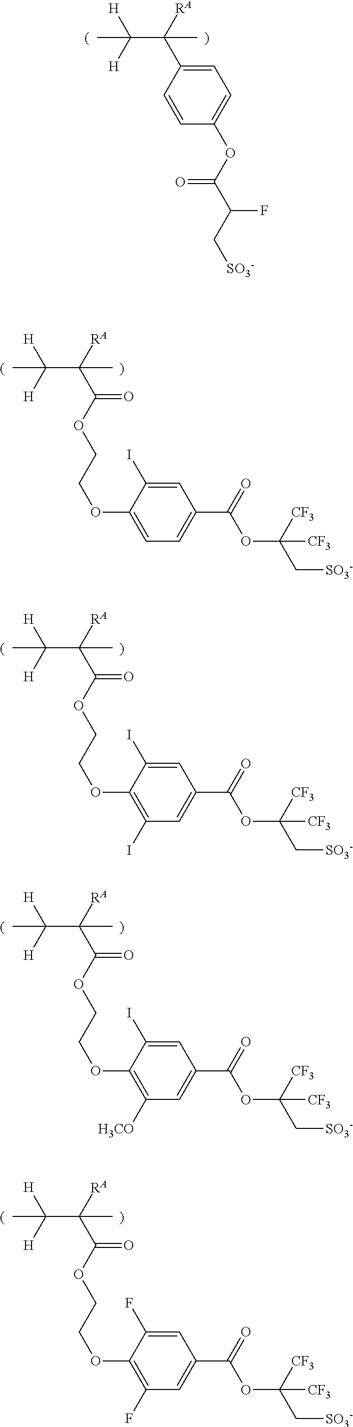

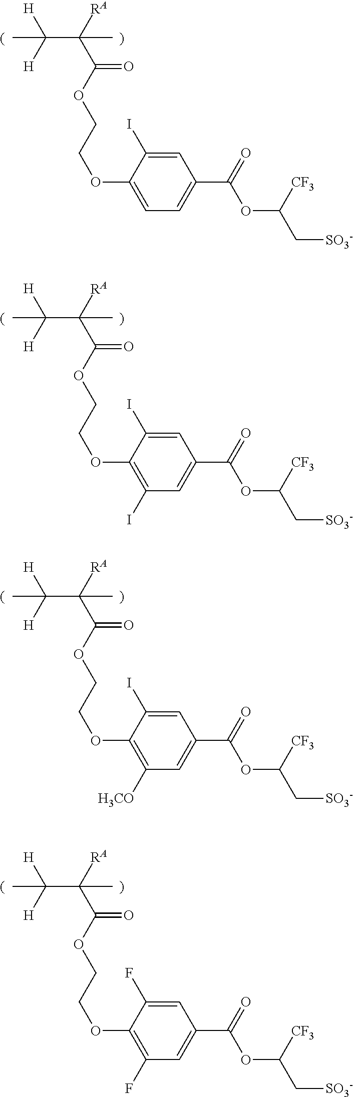

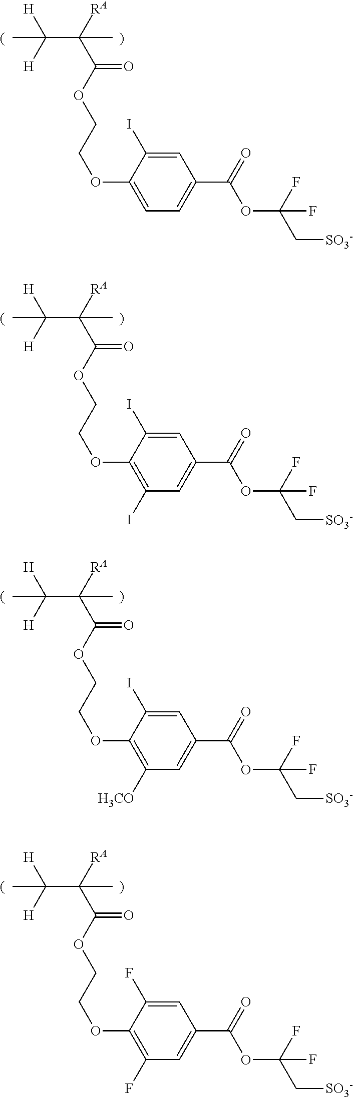

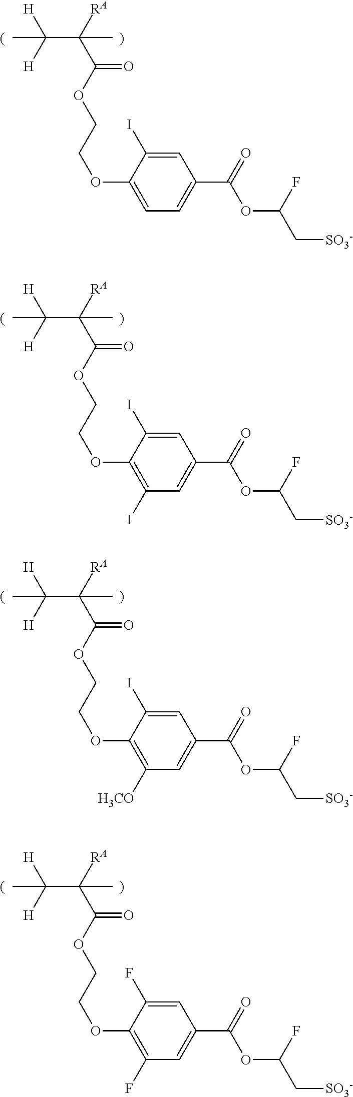

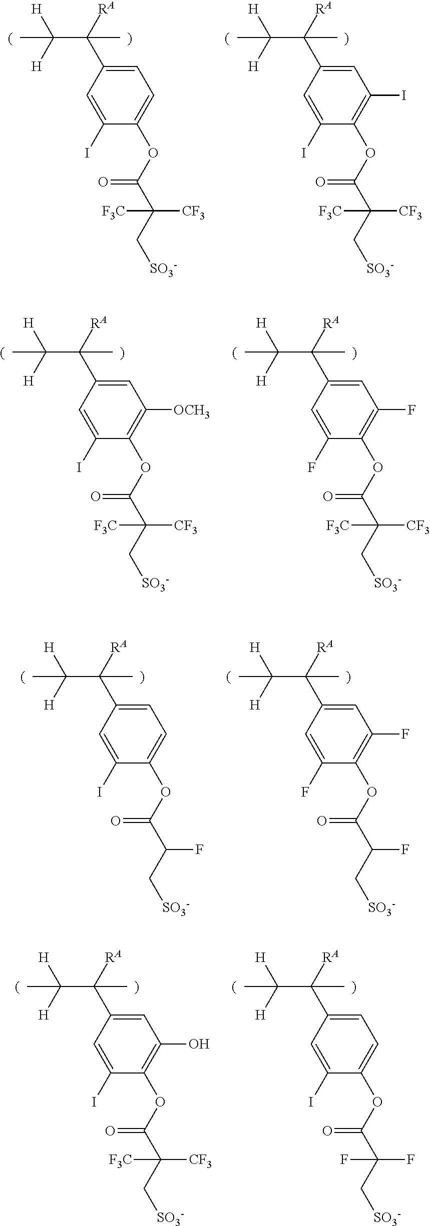

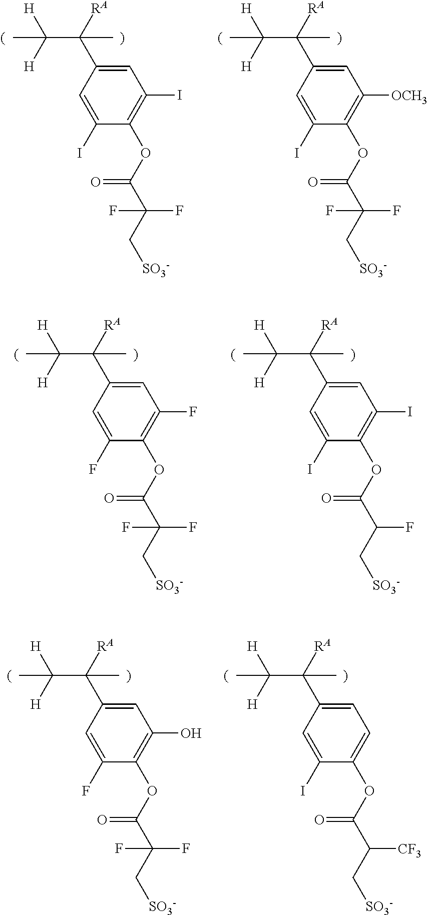

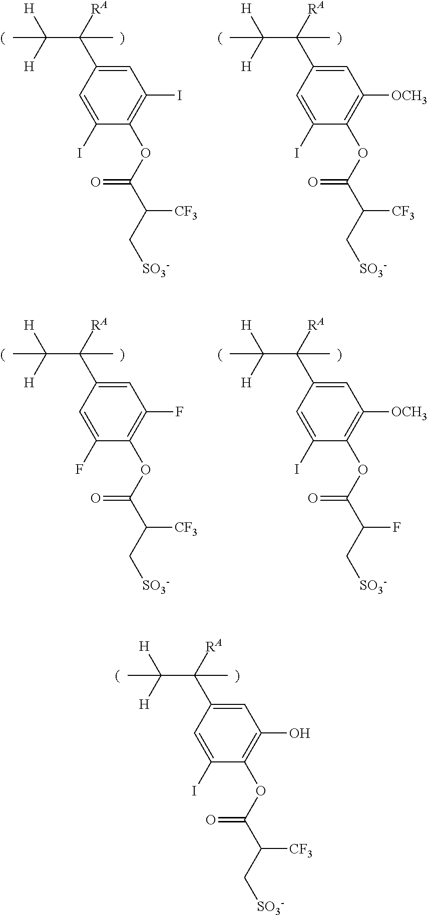

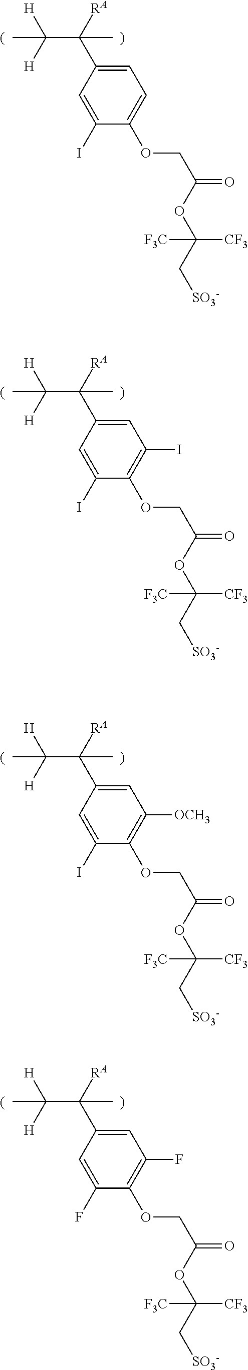

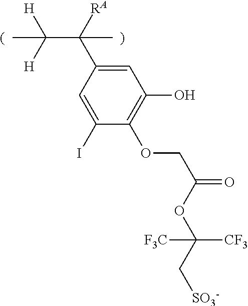

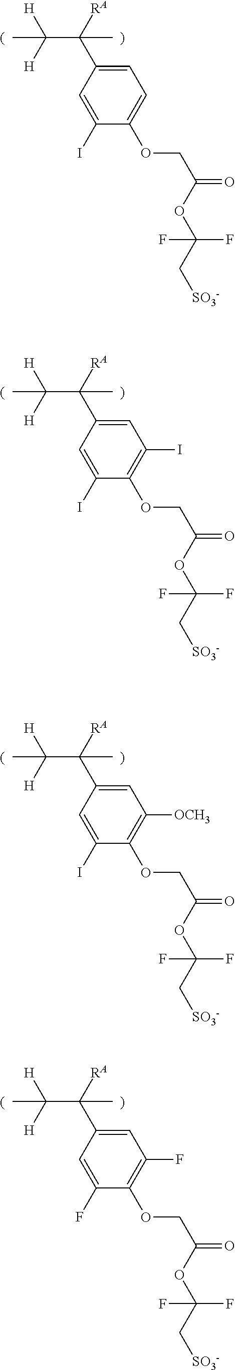

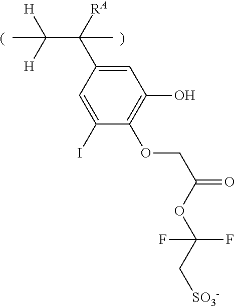

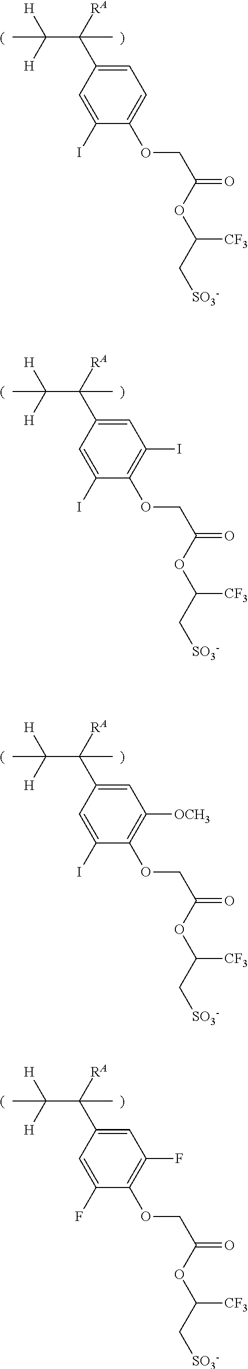

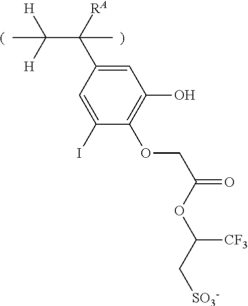

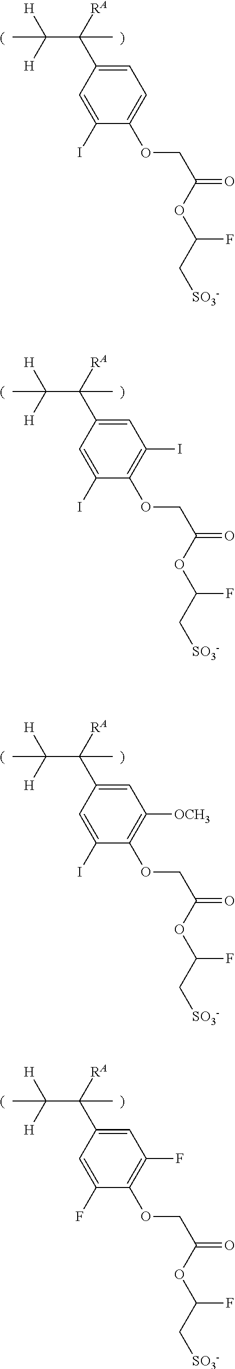

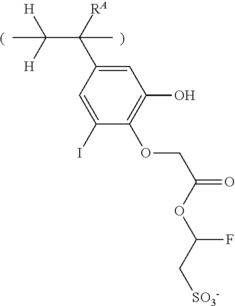

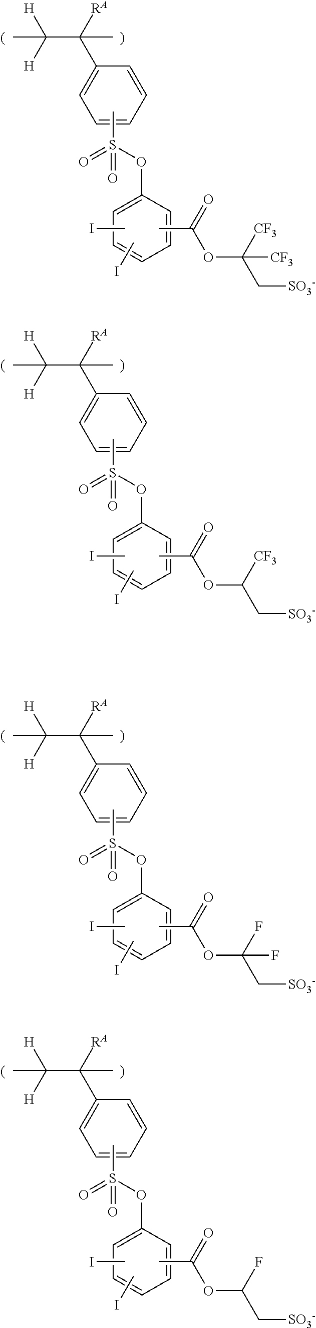

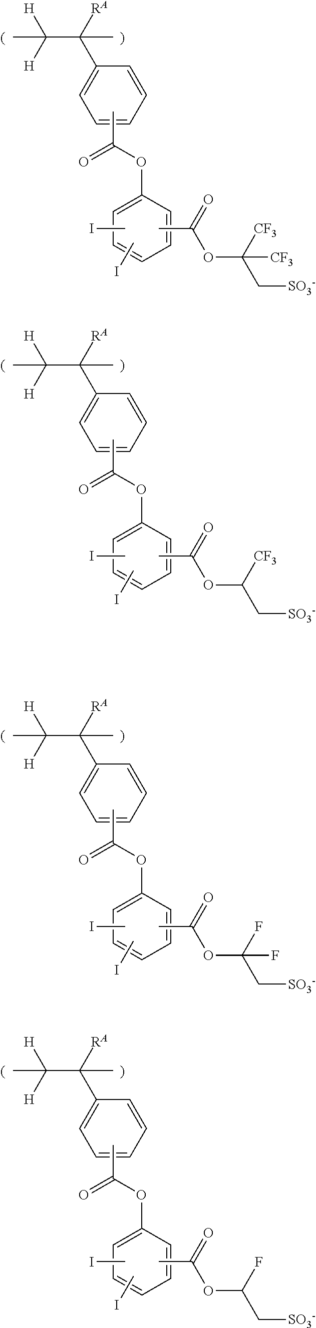

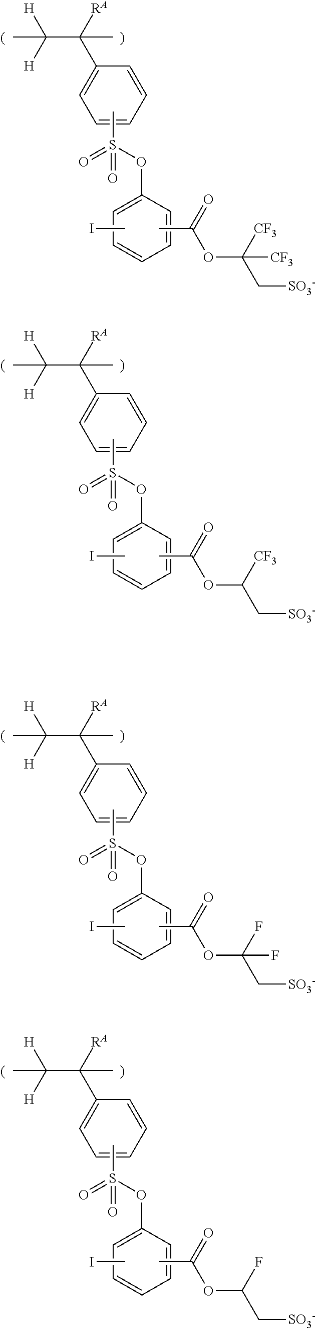

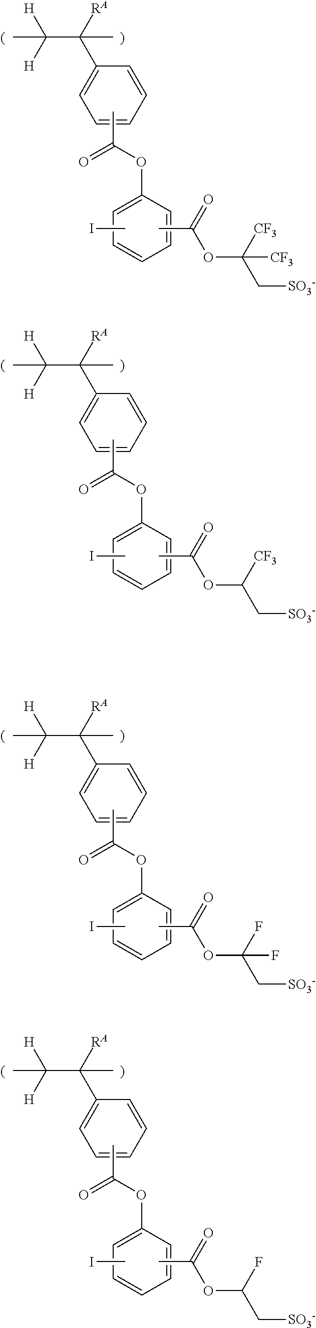

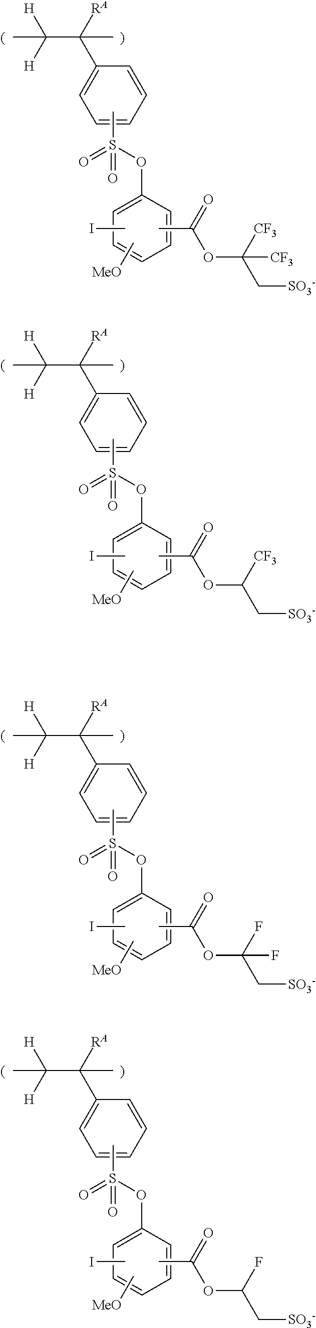

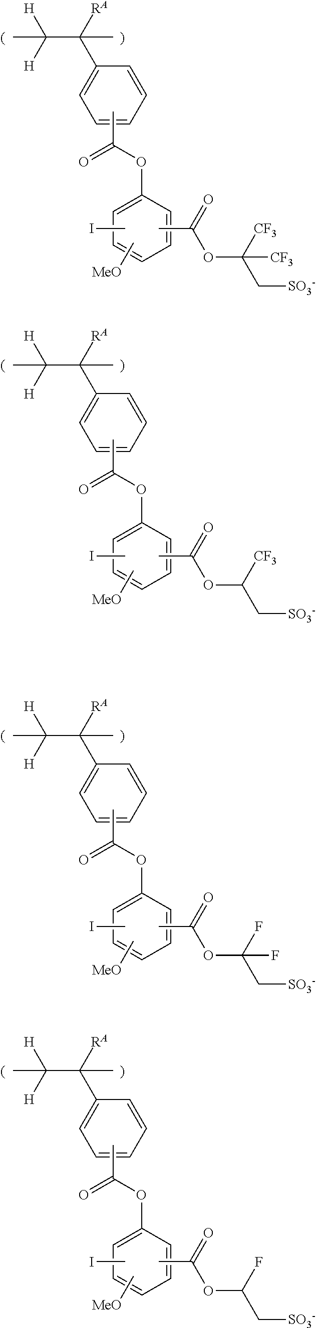

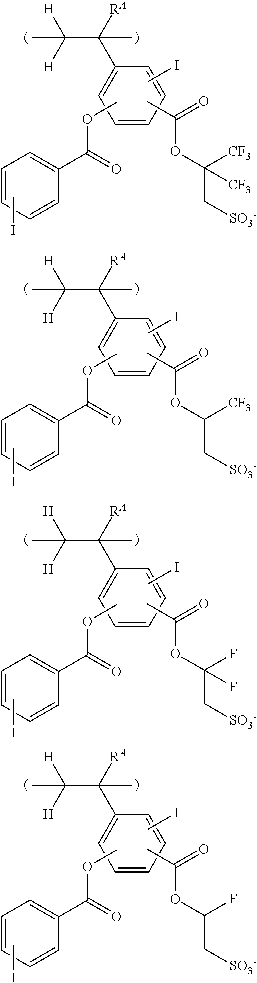

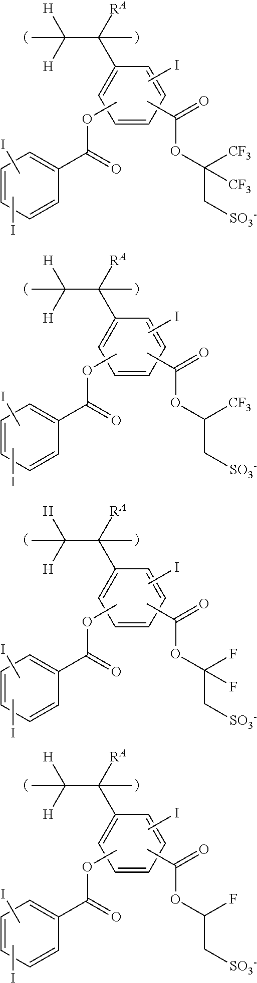

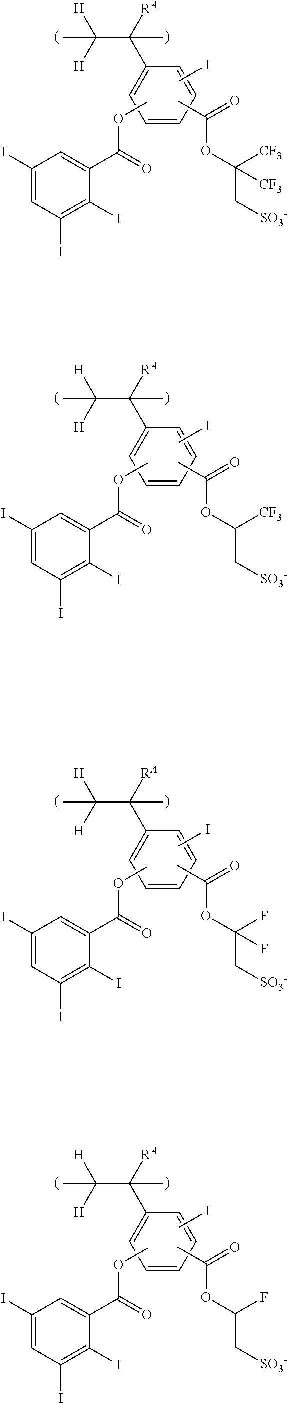

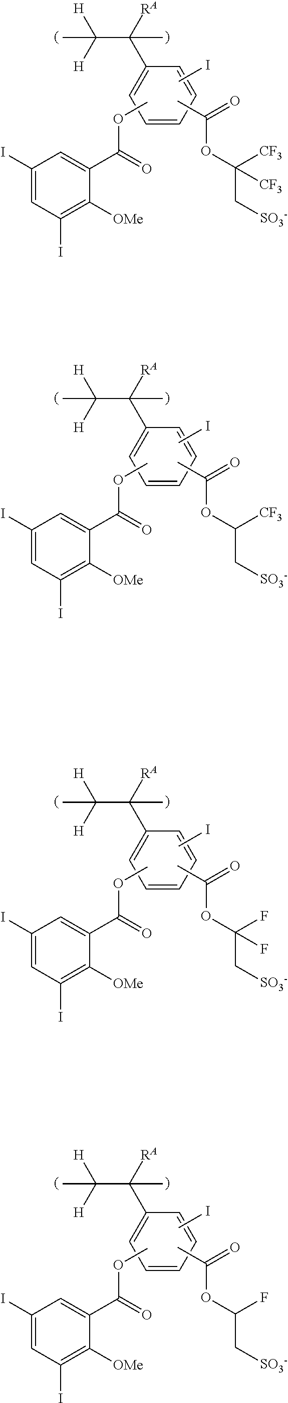

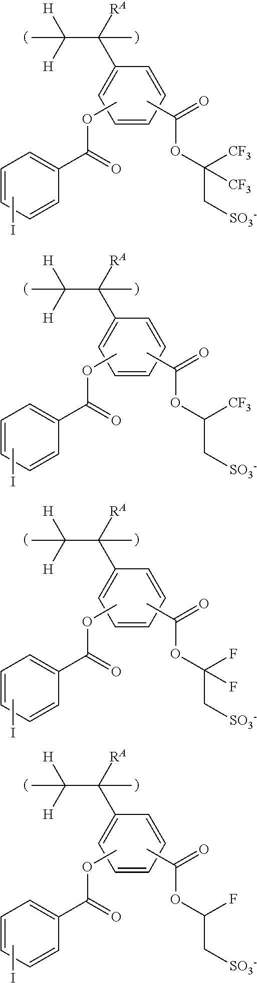

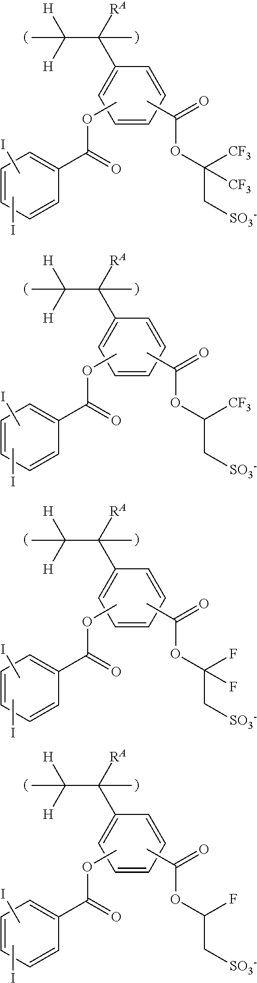

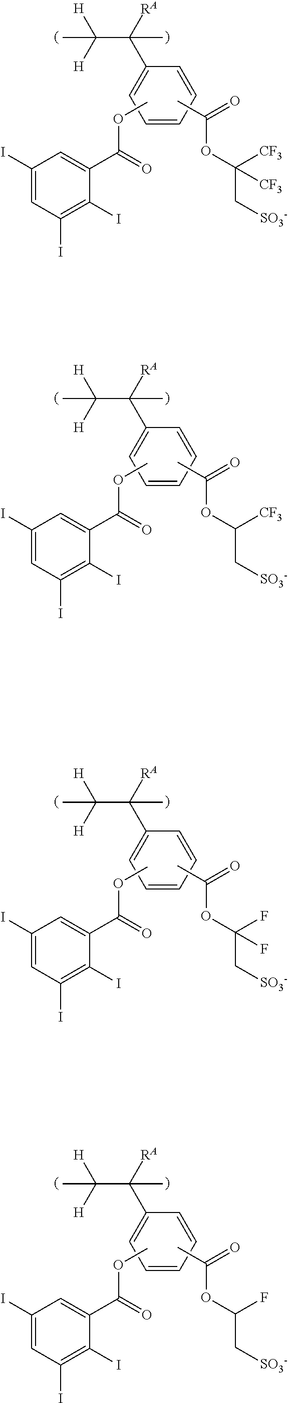

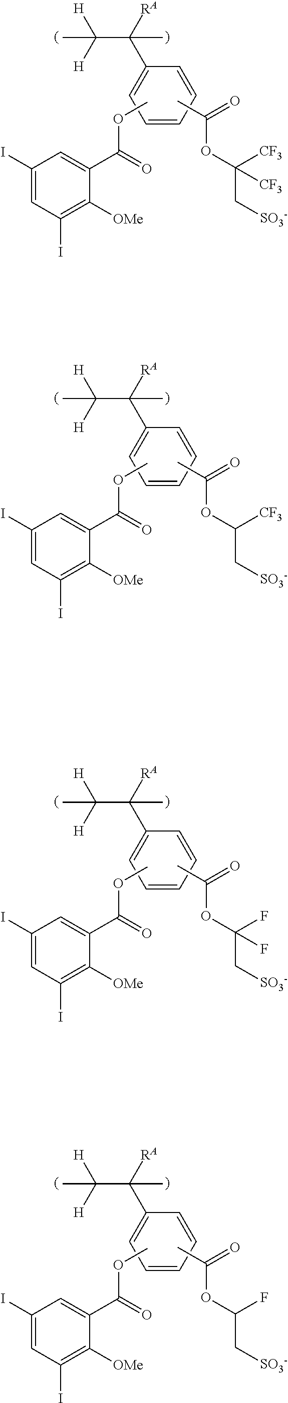

Specific examples of the anion of the repeat unit c4 are shown below, but not limited thereto. Herein R^{A} is as defined above.

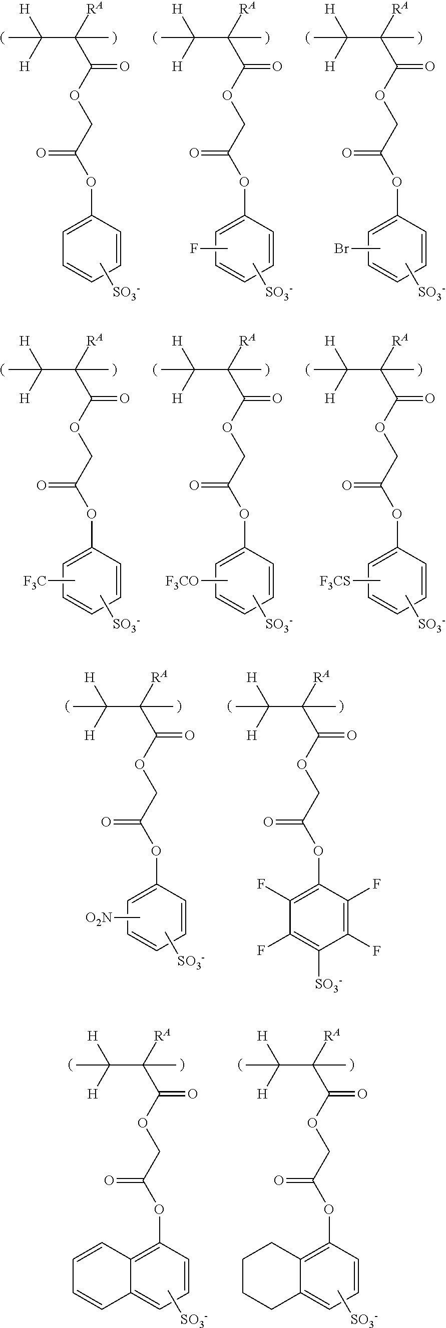

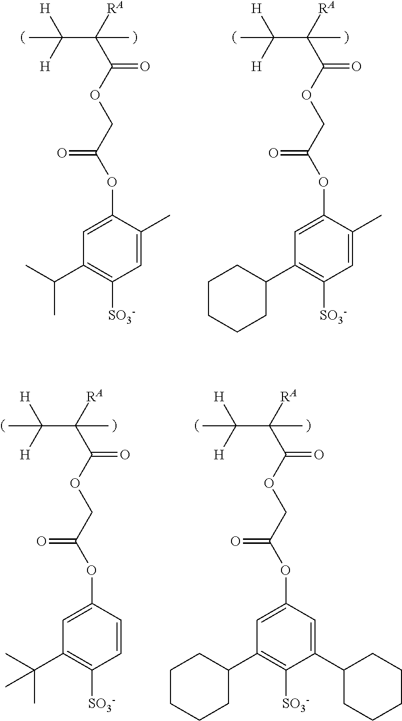

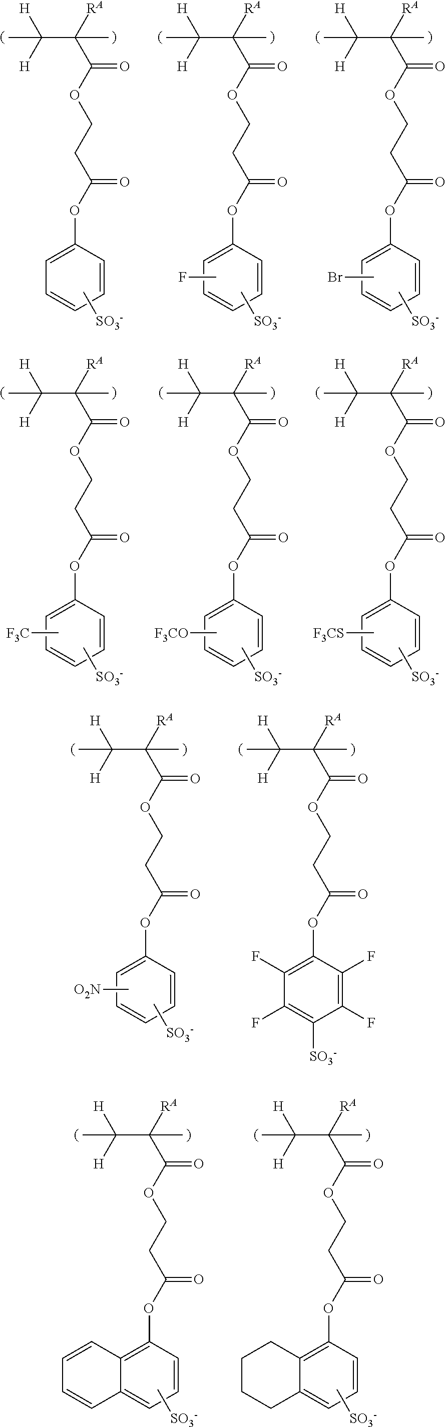

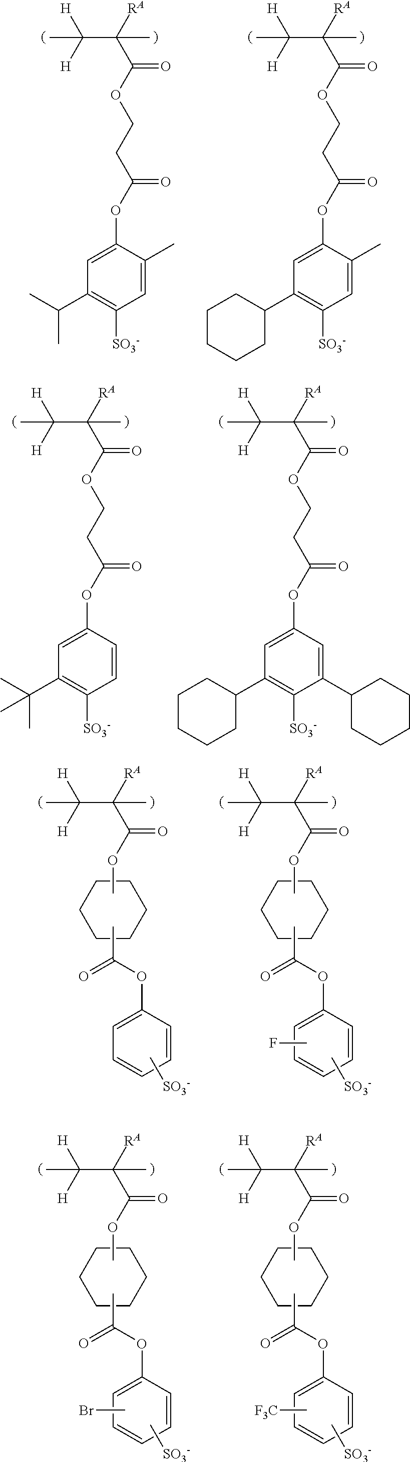

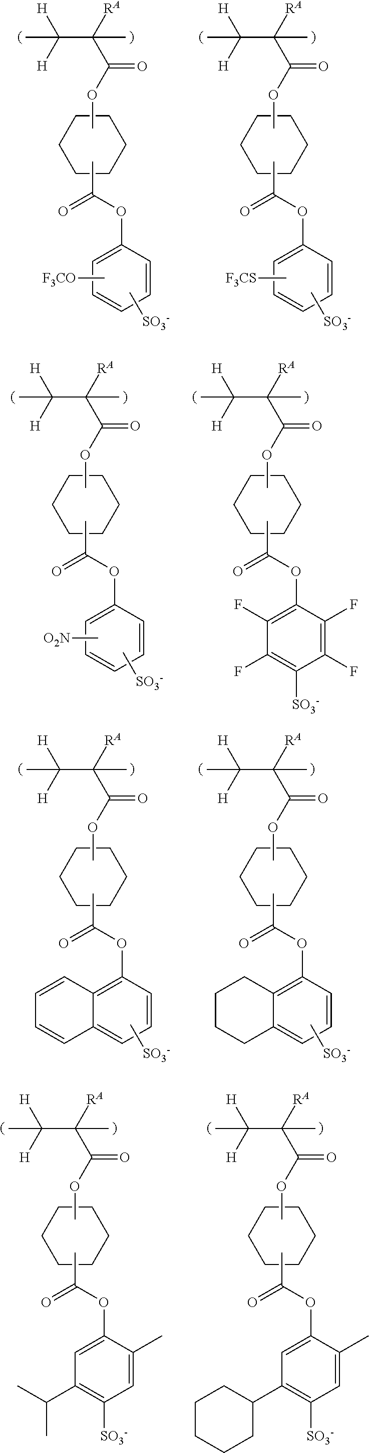

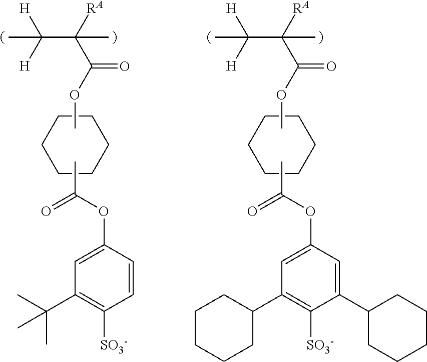

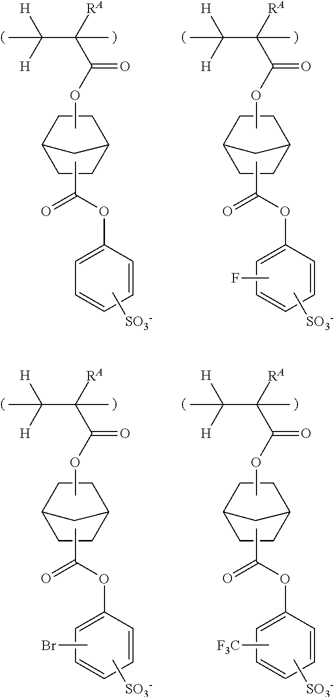

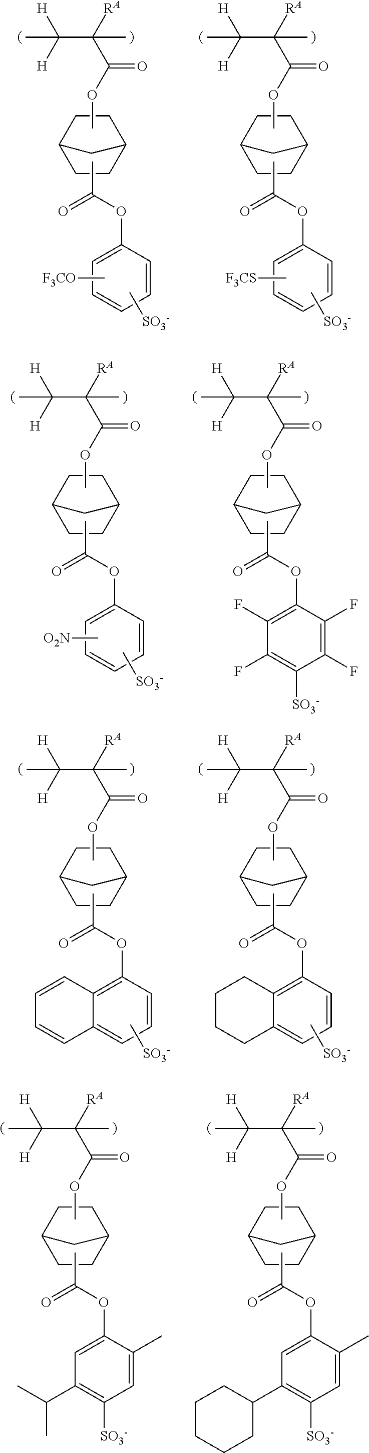

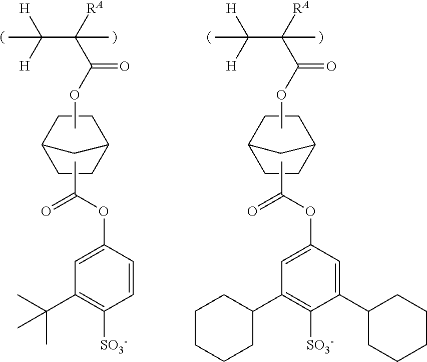

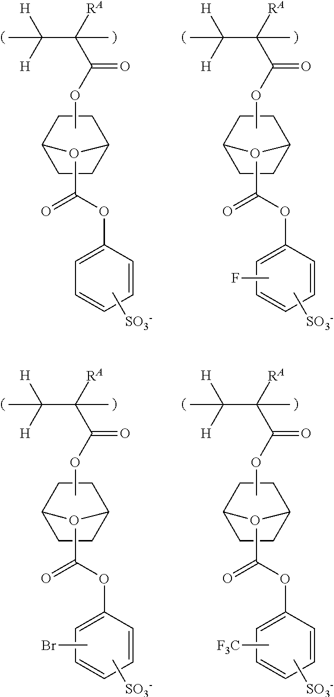

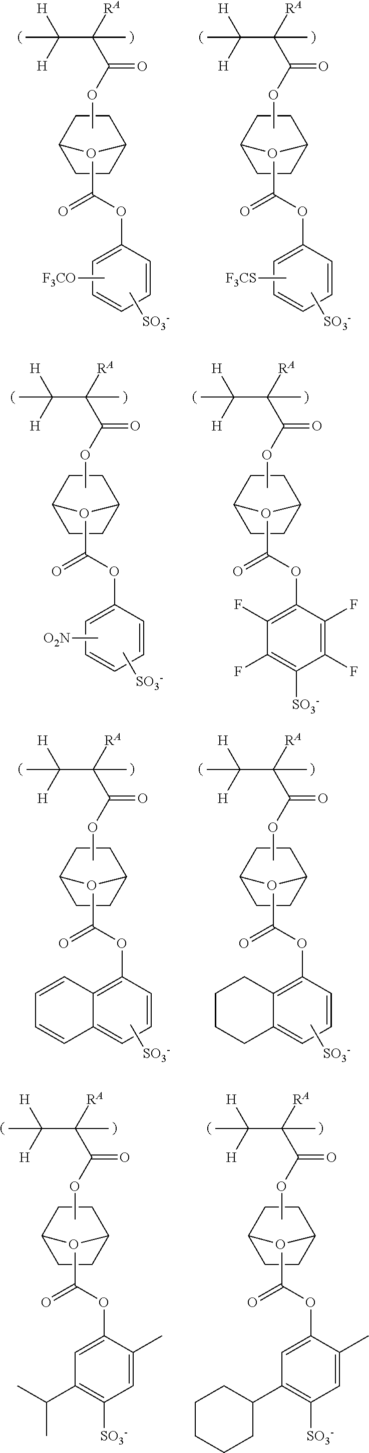

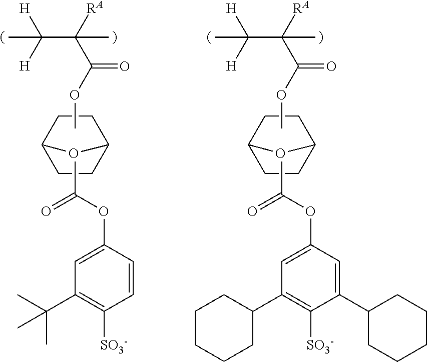

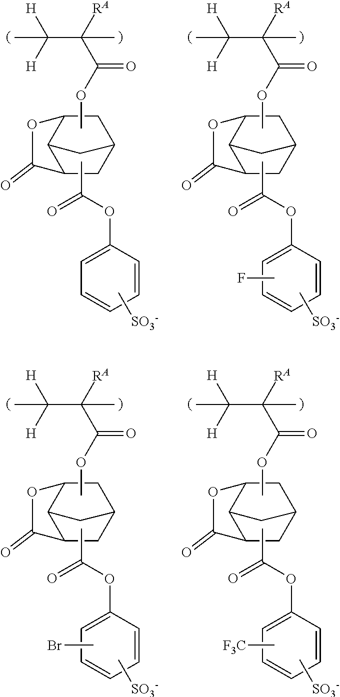

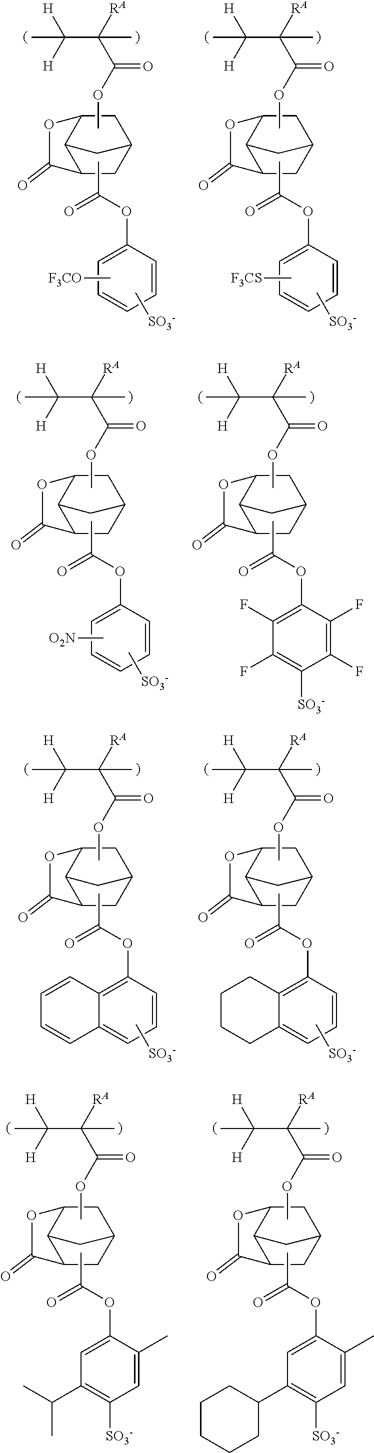

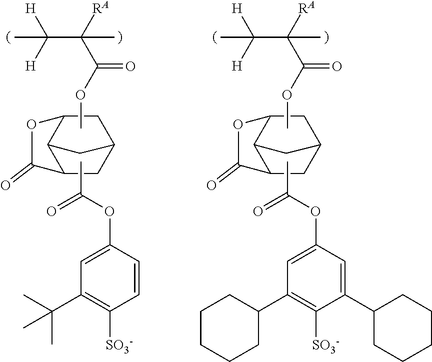

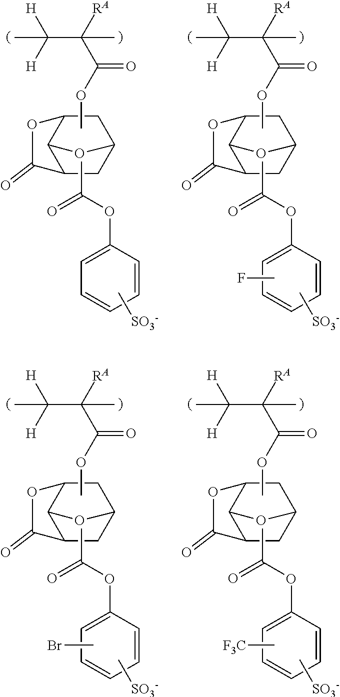

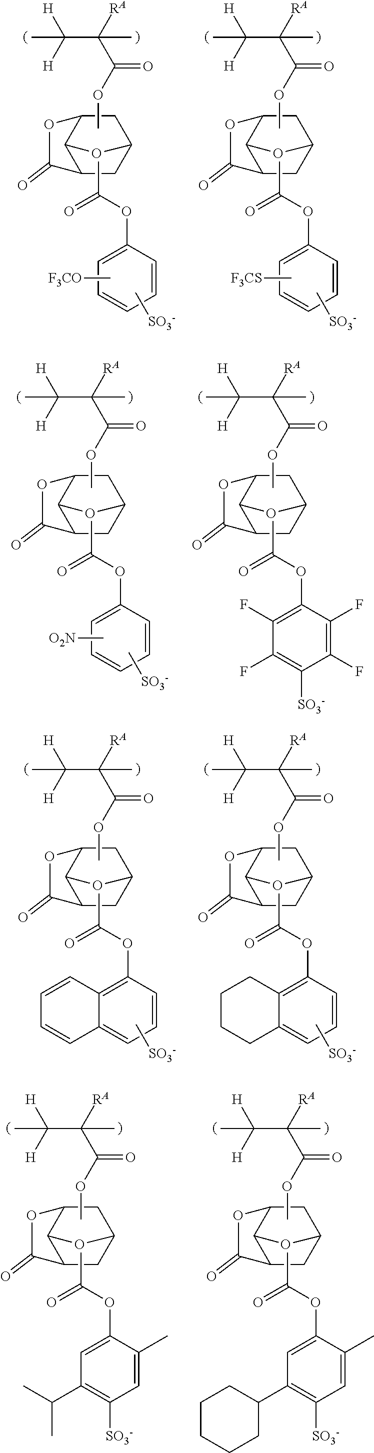

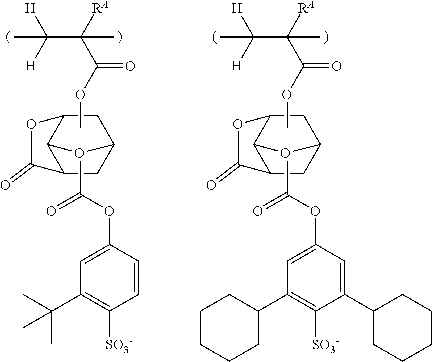

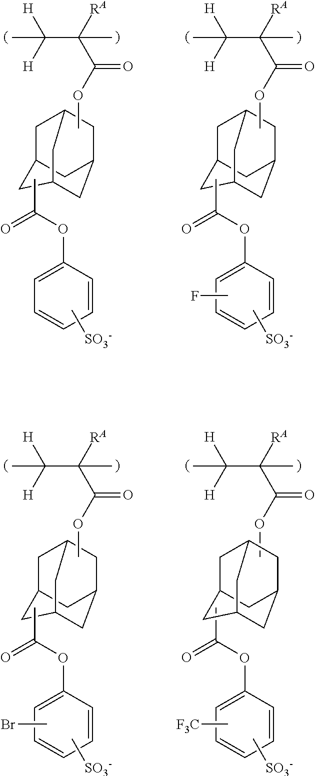

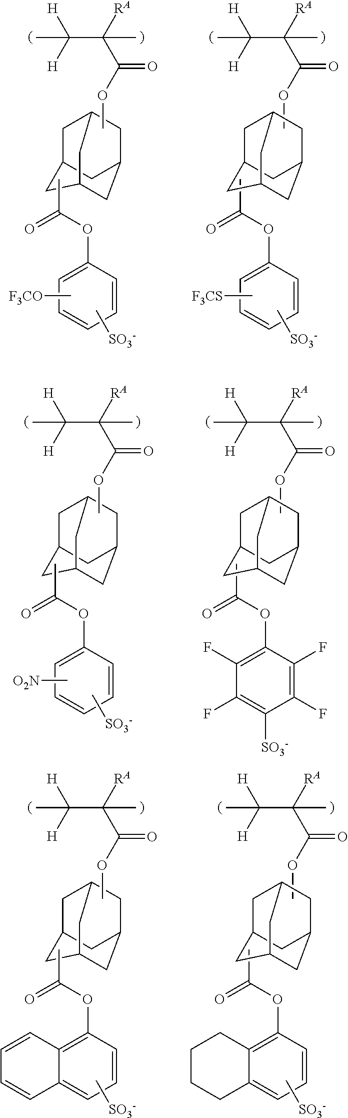

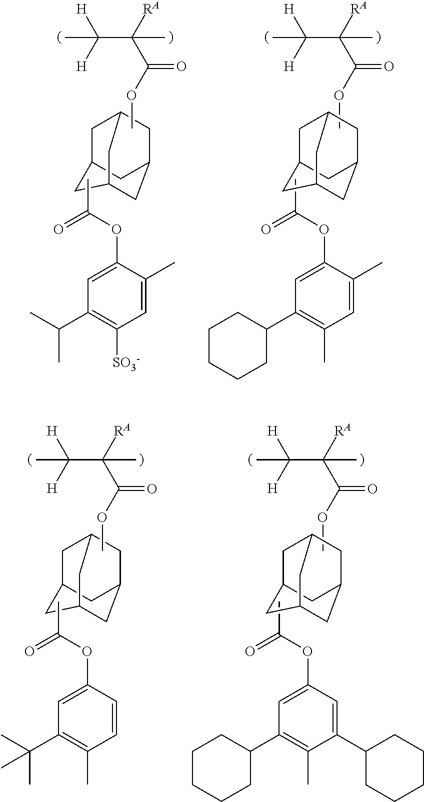

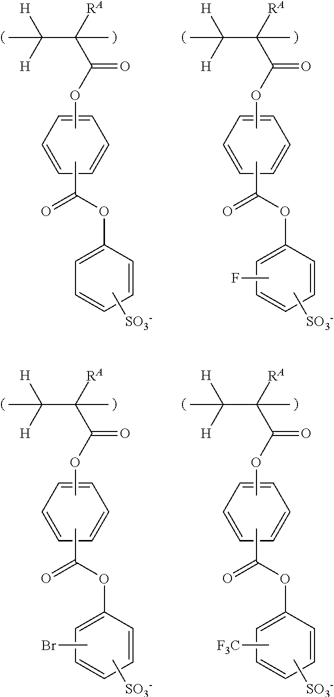

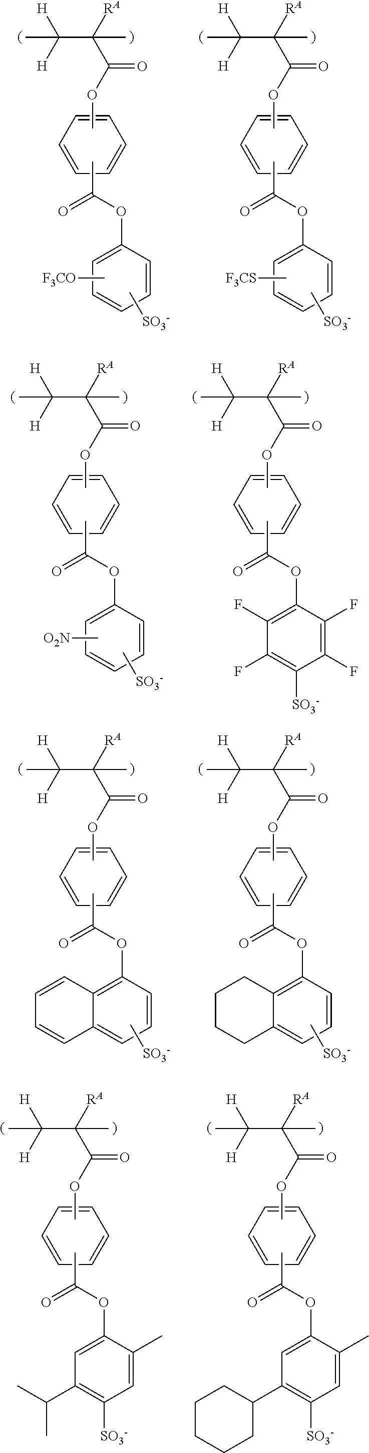

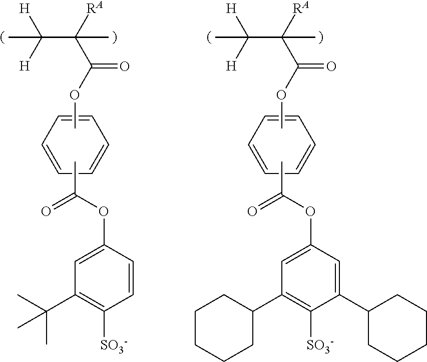

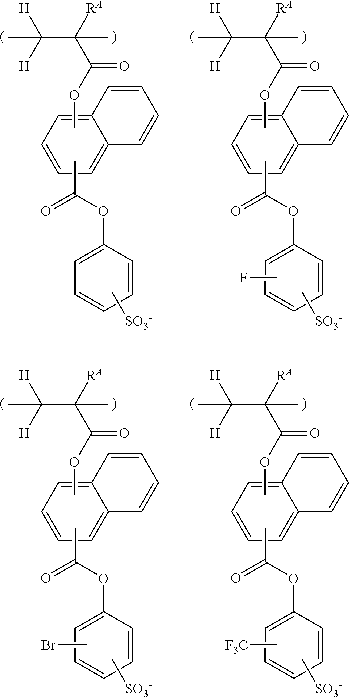

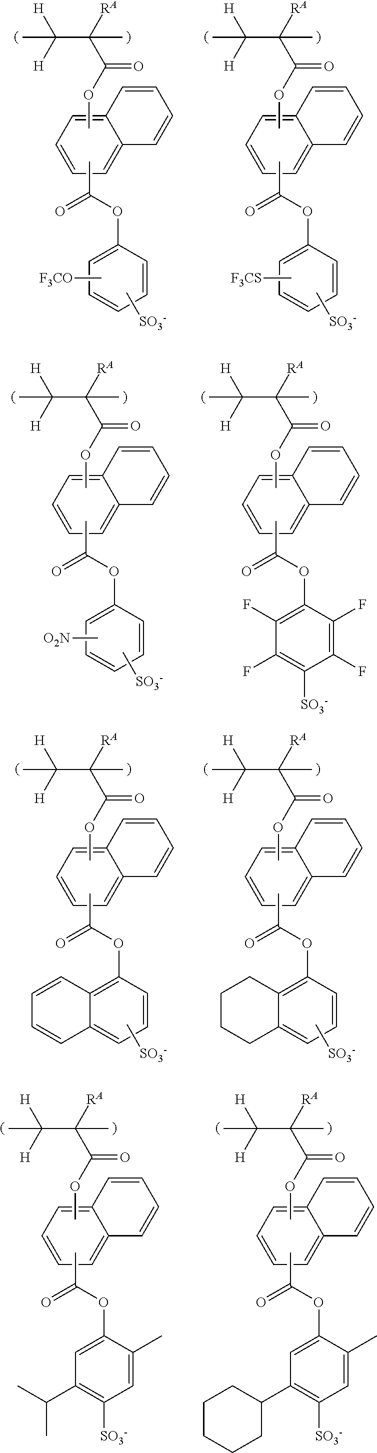

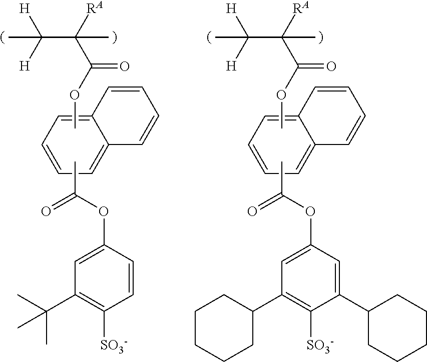

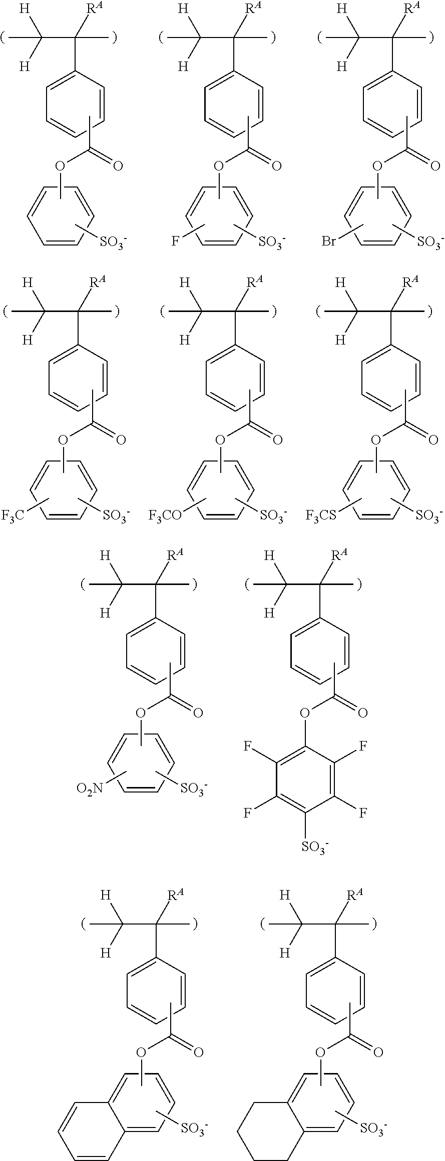

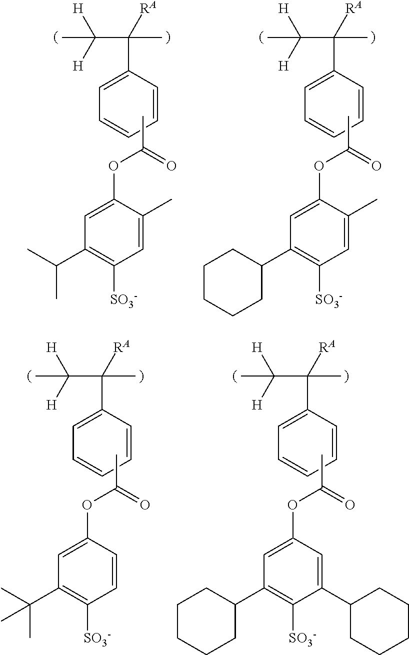

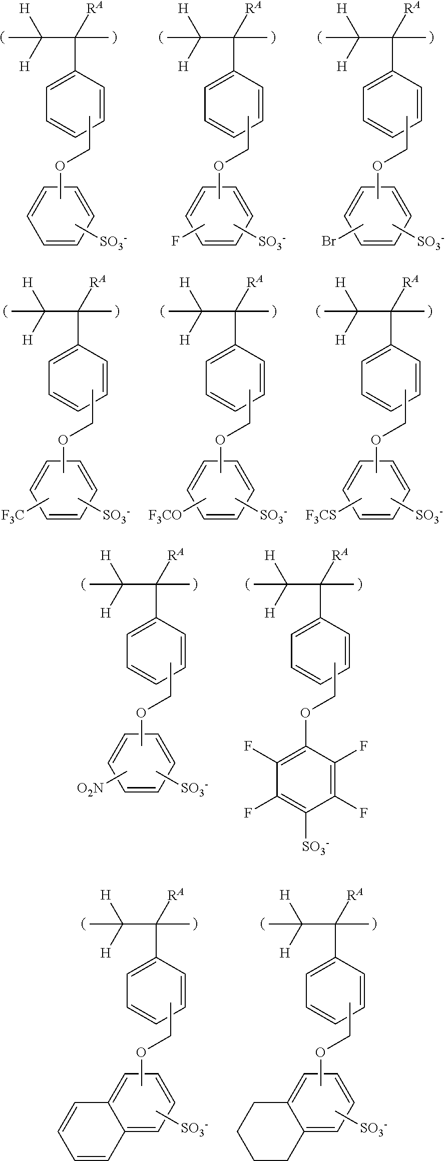

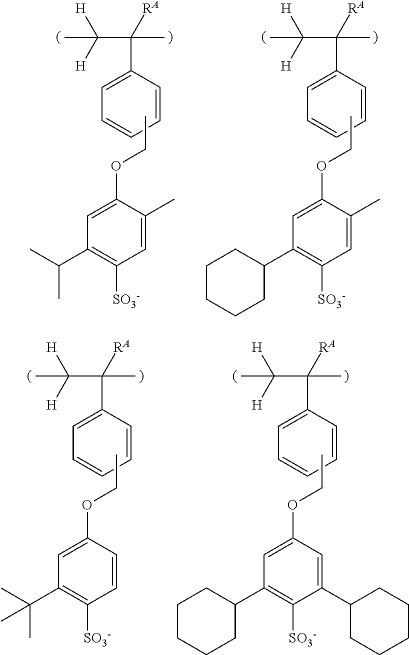

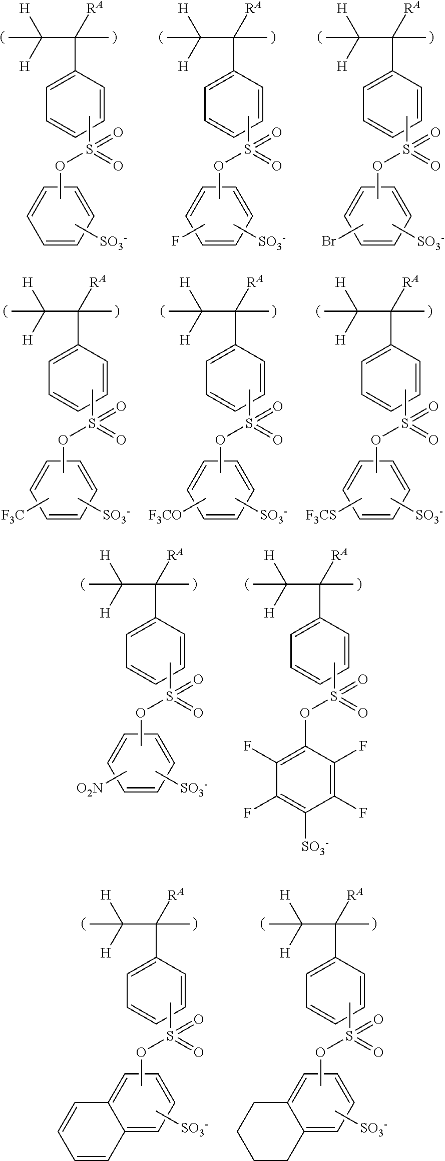

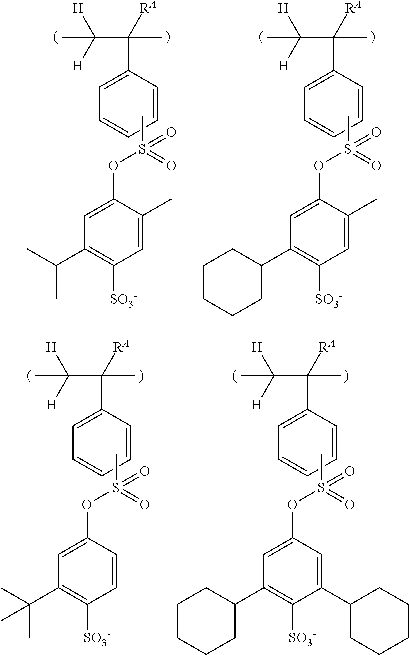

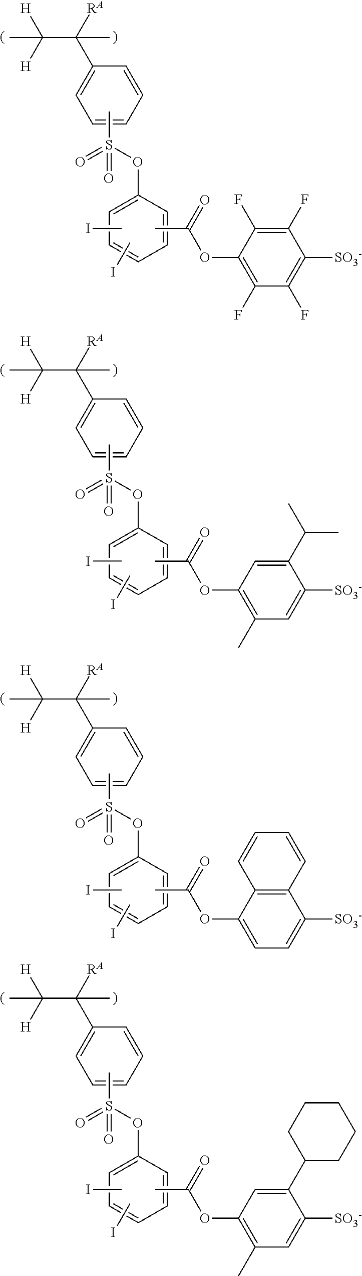

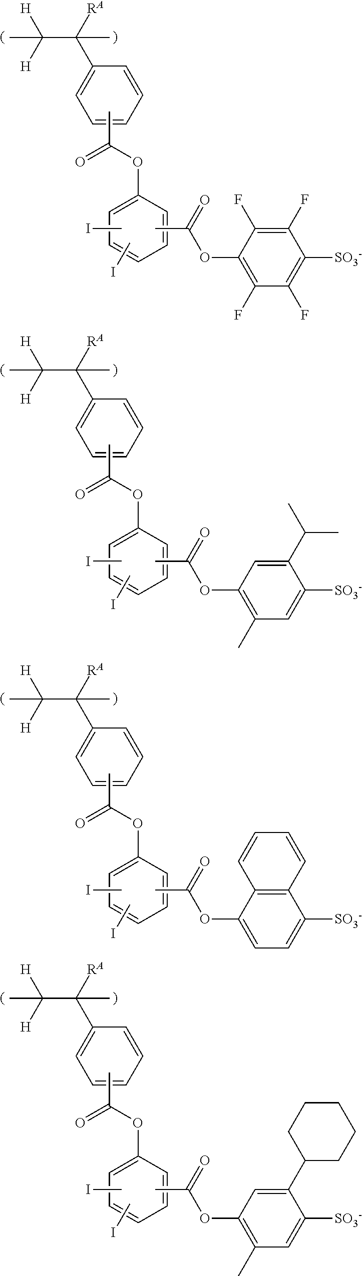

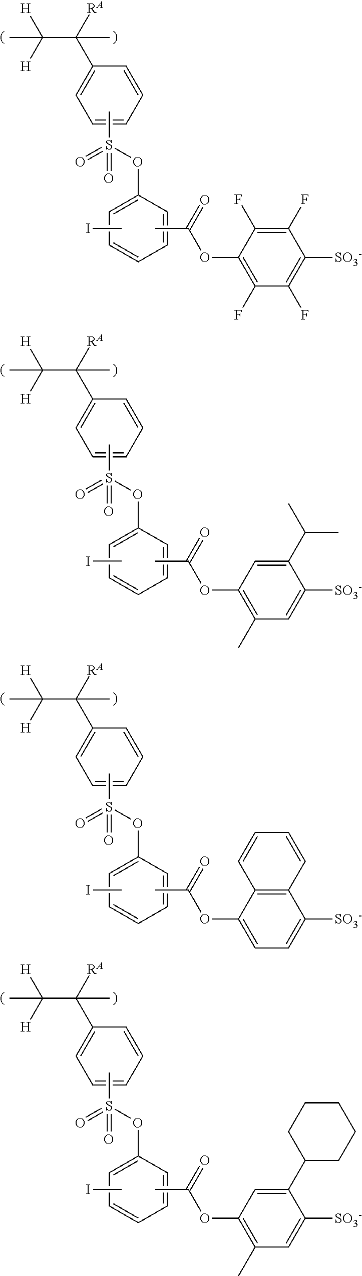

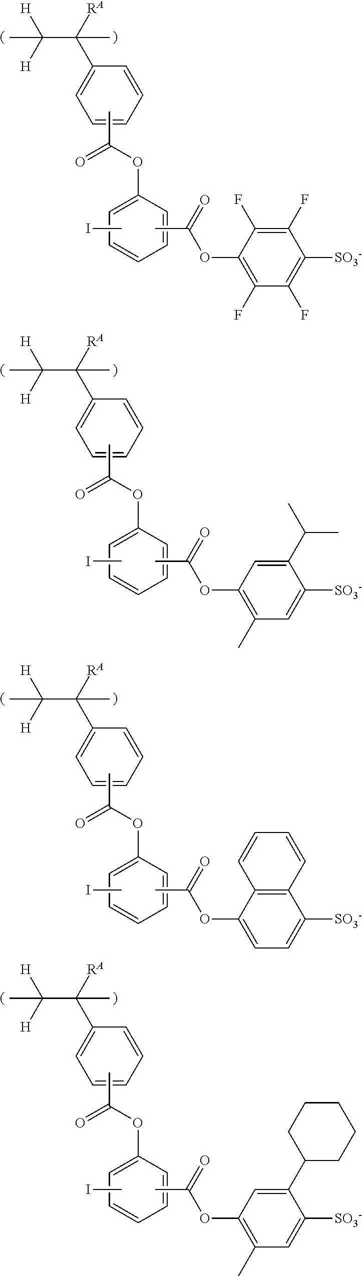

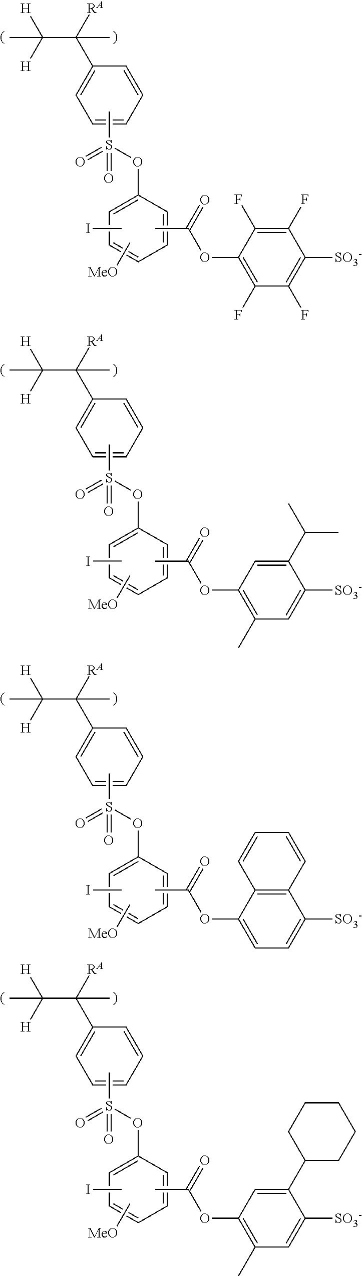

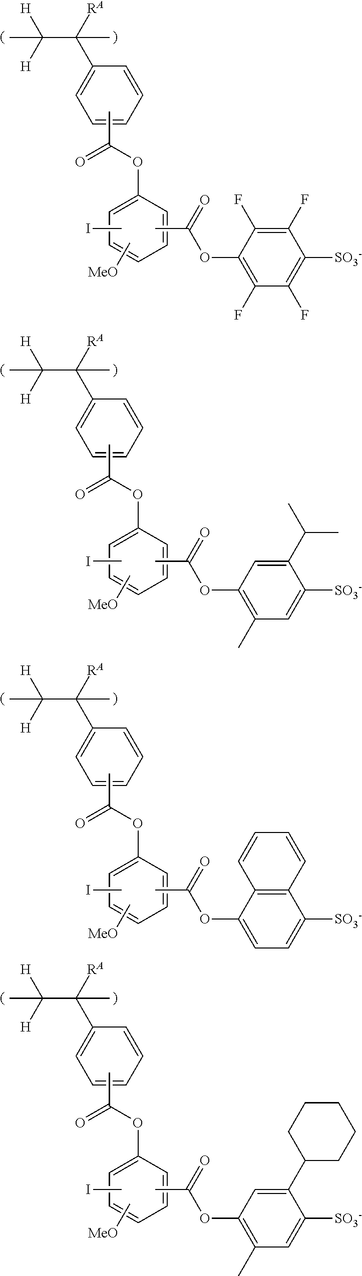

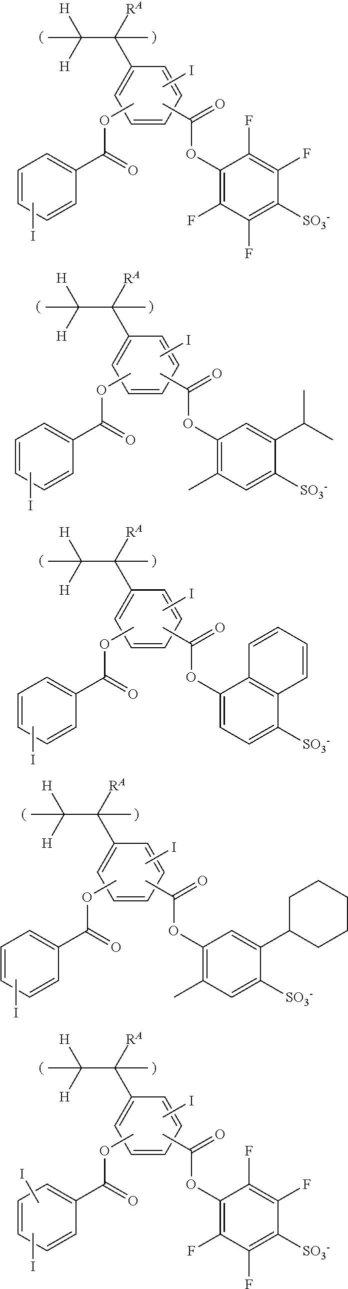

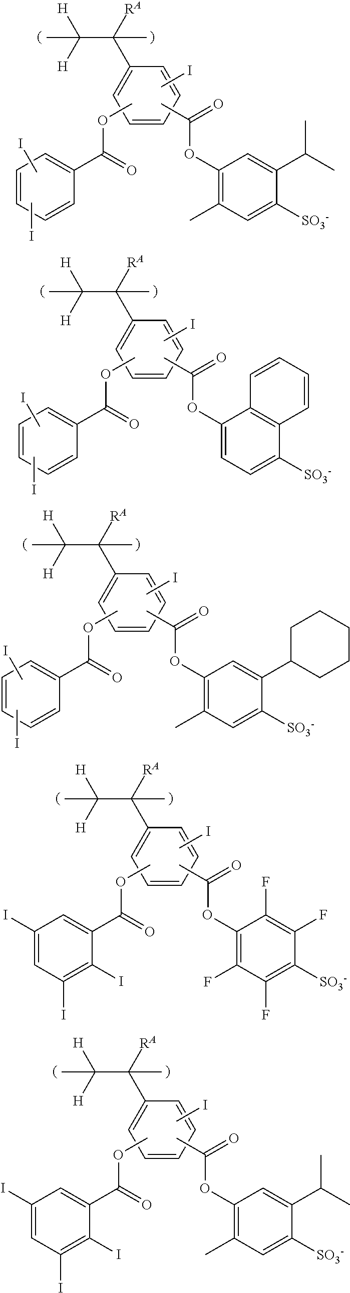

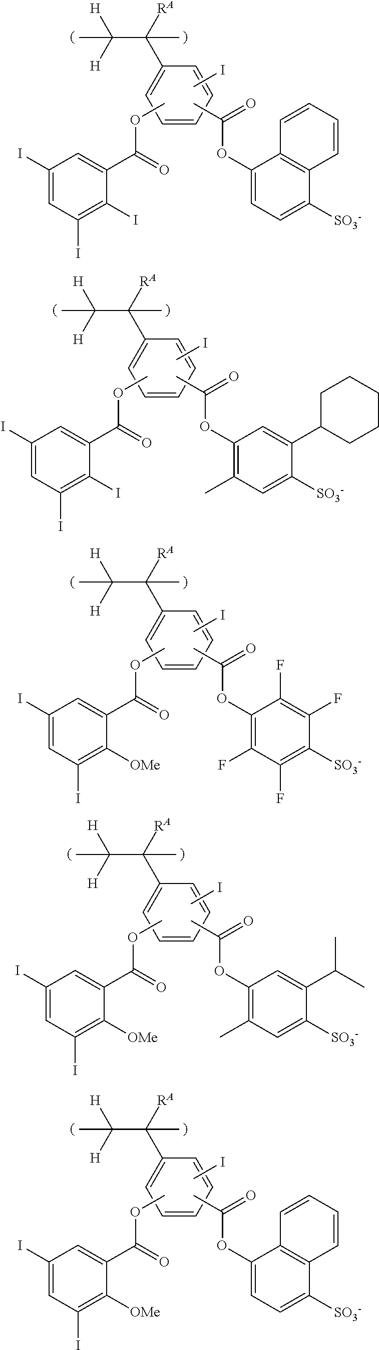

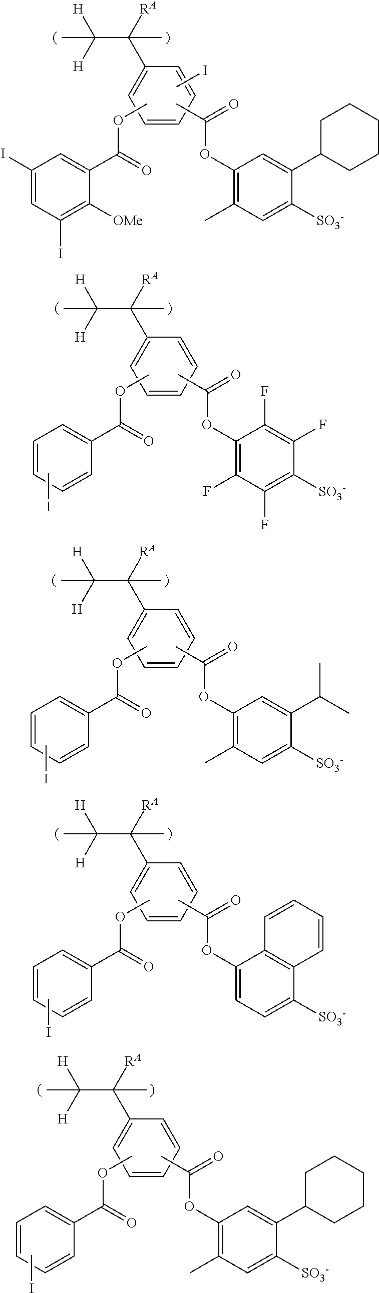

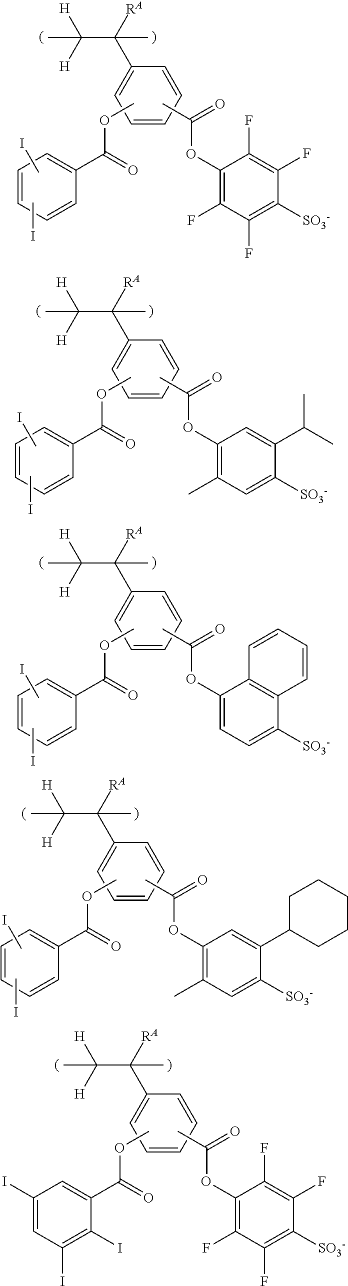

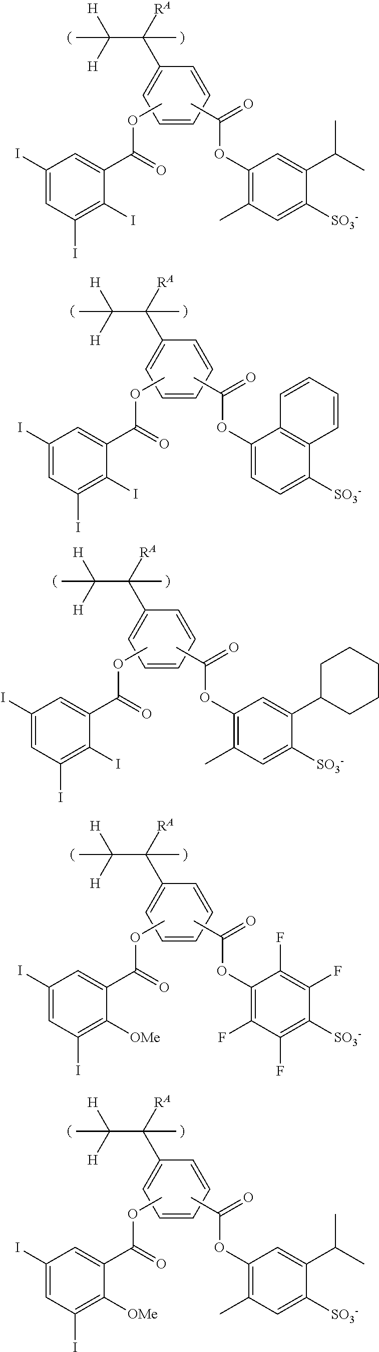

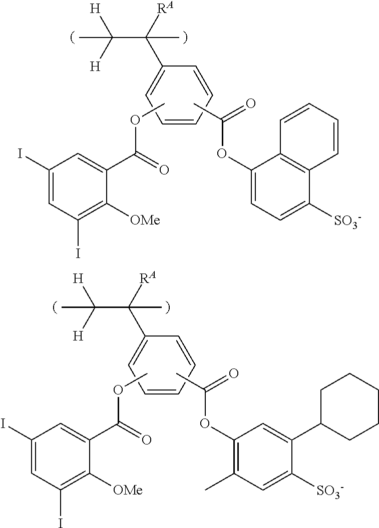

Specific examples of the anion of the repeat unit c5 are shown below, but not limited thereto. Herein R^{A} is as defined above.

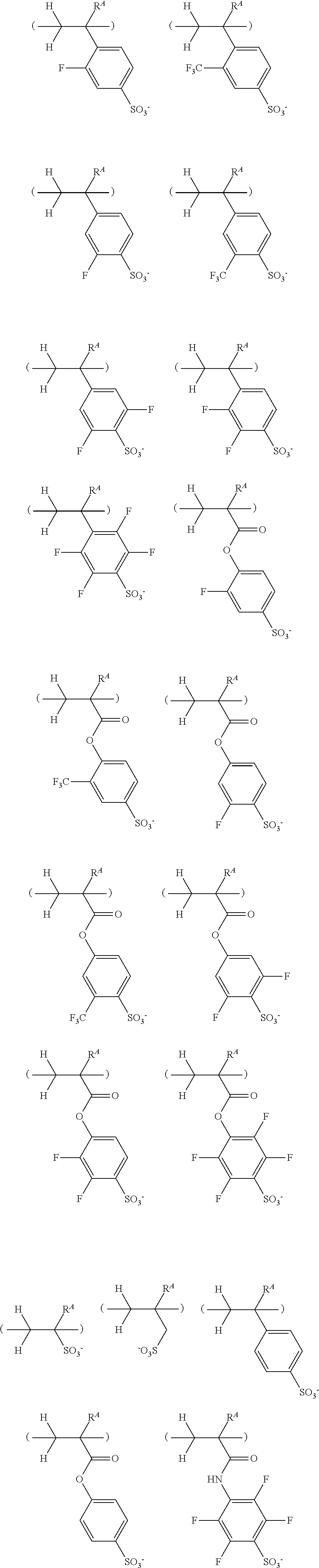

In formulae (c2) to (c5), A⁺ is an onium cation. Examples of the onium cation include a sulfonium cation, an iodonium cation, and an ammonium cation, and a sulfonium cation and an iodonium cation are preferred. Specific examples of the sulfonium cation include those having formula (Z-1), those described in paragraphs to of JP-A 2024-003744, those described in paragraphs to of JP-A 2023-169812, and those having formula (Z-3), but not limited thereto. Specific examples of the iodonium cation include those having formula (Z-2) and those described in paragraph of JP-A 2024-000259, but not limited thereto. Specific examples of the ammonium cation are as exemplified above for the ammonium cation having the formula (am-1), but not limited thereto.

Specific structures of the repeat units c1 to c5 include any combination of the foregoing anion and cation.

Of the repeat units c1 to c5, the repeat units c2 to c5 are preferred from the viewpoint of controlling acid diffusion, the repeat units c2, c4, and c5 are further preferred from the viewpoint of acid strength of the generated acid, and the repeat unit c2 is more preferred from the viewpoint of solvent solubility.

The base polymer may further comprise a repeat unit having a structure in which a hydroxy group is protected with an acid labile group (hereinafter, the repeat unit is also referred to as a repeat unit d). The repeat unit d is not particularly limited as long as it has one or two or more structures in which a hydroxy group is protected, and a protective group is decomposed to generate the hydroxy group under the action of acid. A repeat unit having the formula (d1) is preferred.

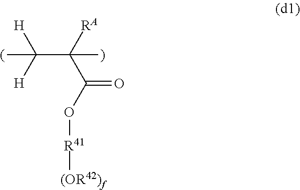

In formula (d1), R^{A} is as defined above. R⁴¹ is a C₁-C₃₀ (f+1)-valent hydrocarbon group which may contain a heteroatom. R⁴² is an acid labile group. f is 1, 2, 3, or 4.

In formula (d1), the acid labile group R⁴² is deprotected under the action of acid so that a hydroxy group is generated. The structure of R⁴² is not particularly limited, an acetal structure, a ketal structure, an alkoxycarbonyl group, and an alkoxymethyl group having the formula (d2) are preferred, and an alkoxymethyl group having the formula (d2) is particularly preferred.

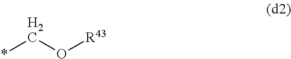

Herein * designates a point of attachment and R⁴³ is a C₁-C₁₅ hydrocarbyl group.

Specific examples of the acid labile group R⁴², the alkoxymethyl group having formula (d2), and the repeat unit d are as exemplified for the repeat unit d in JP-A 2020-111564.

The base polymer may further comprise a repeat unit e derived from indene, benzofuran, benzothiophene, acenaphthylene, chromone, coumarin, norbornadiene, and derivatives thereof. Specific examples of the monomer from which the repeat unit e is derived are shown below, but not limited thereto.

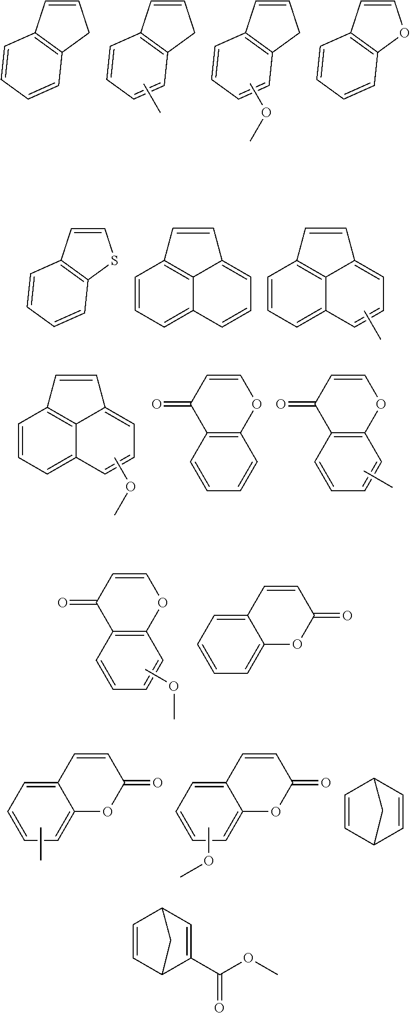

The base polymer may further comprise a repeat unit f derived from indane, vinylpyridine, or vinylcarbazole.

In the present polymer, repeat units a1, a2, a3, b1, b2, c1 to c5, d, e, and f are incorporated in a ratio of preferably 0<a1 ≤0.8,0≤a2≤0.8,0≤a3≤0.6,0≤b1≤0.6, 0≤b2≤0.6, 0≤c1 ≤0.4, 0≤c2≤0.4, 0≤c3≤0.4, 0≤c4≤0.4, 0≤c5≤0.4, 0≤d≤0.5, 0≤e≤0.3, and 0≤f≤0.3; more preferably 0<a1 ≤0.7, 0≤a2≤0.7, 0≤a3≤0.5, 0≤b1≤0.5, 0≤b2≤0.5, 0≤c1 ≤0.3, 0≤c2≤0.3, 0≤c3≤0.3, 0≤c4≤0.3, 0≤c5≤0.3, 0≤d≤0.3, 0≤e≤0.3, and 0≤f≤0.3. Provided that a1+a2+a3+b1+b2+c1+c2+c3+c4+d+e+f≤1.0.

A weight average molecular weight (Mw) of the polymer is preferably 1,000 to 500,000, more preferably 3,000 to 100,000. If the Mw is in this range, sufficient etch resistance is obtained, and there is no possibility of resolution decline due to inability to secure a difference in dissolution rate before and after exposure. In the invention, the Mw is a value measured in terms of polystyrene by gel permeation chromatography (GPC) using THF or N,N-dimethylformamide (DMF) as a solvent.

In a molecular weight dispersity (Mw/Mn) of the polymer, the influence of the Mw/Mn tends to increase as the pattern rule becomes finer. Therefore, in order to obtain a resist composition suitably used for micropatterning to a small feature size, the Mw/Mn preferably has a narrow dispersity of 1.0 to 2.0. Within the above range, the contents of lower and higher molecular weight polymer fractions are low, and there is no possibility that foreign matter is observed on the pattern or the shape of the pattern is degraded after exposure.

In order to synthesize the polymer, for example, a monomer that provides the foregoing repeat units may be dissolved in an organic solvent, a radical polymerization initiator is added, and heating is performed for polymerization.

Specific examples of the organic solvent used in the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours in view of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form an ultra high molecular-weight polymer, it is preferred from the viewpoint of quality control to independently prepare the monomer solution and the initiator solution separately and add them dropwise. The acid labile group that has been incorporated in the monomer may be kept as such, or polymerization may be followed by protection or partial protection. During the polymer synthesis, any known chain transfer agent such as dodecyl mercaptan or 2-mercaptoethanol may be added for molecular weight control purpose. The amount of chain transfer agent added is preferably 0.01 to 20 mol % based on the total of monomers.

When a hydroxy-containing monomer is copolymerized, the hydroxy group is substituted with an acetal group which is susceptible to deprotection with acid, typically ethoxyethoxy, prior to polymerization, and the polymerization is followed by deprotection with weak acid and water. Alternatively, the hydroxy group is substituted with an acetyl, formyl or pivaloyl group prior to polymerization, and the polymerization is followed by alkaline hydrolysis.

When hydroxystyrene or hydroxyvinylnaphthalene is copolymerized, one method is dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or polyhydroxyvinylnaphthalene.

For alkaline hydrolysis, a base such as aqueous ammonia or triethylamine may be used. The reaction temperature is preferably-20 to 100° C., more preferably 0 to 60° C. The reaction time is preferably 0.2 to 100 hours, more preferably 0.5 to 20 hours.

The amounts of monomers in the monomer solution may be determined appropriate so as to provide the preferred fractions of repeat units.

It is now described how to use the polymer obtained by the above preparation method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the viewpoints of operation efficiency and consistent quality to handle a polymer solution which is obtained by dissolving the powder polymer resulting from the purifying step in a solvent, as the final product.

Specific examples of the solvents which can be used herein are those described in paragraphs [0144]-[0145] of JP-A 2008-111103, including ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); high-boiling alcohols such as diethylene glycol, propylene glycol, glycerol, 1,4-butanediol, and 1,3-butanediol; and mixtures thereof.

The polymer solution preferably has a polymer concentration of 0.01 to 30 wt %, more preferably 0.1 to 20 wt %.

The reaction solution or the polymer solution is preferably subjected to filter filtration. By performing filter filtration, foreign matter and gels that may cause defects can be removed, and it is effective in terms of quality stabilization.

Examples of the material of the filter used for the filter filtration include fluorocarbon, cellulose, nylon, polyester, and hydrocarbon base materials, and in the filtration of a resist composition, filters formed of fluorocarbons commonly known as Teflon®, hydrocarbons such as polyethylene and polypropylene, and nylon are preferred. The pore size of the filter can be appropriately selected according to the desired cleanness, and is preferably 100 nm or less, more preferably 20 nm or less. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The base polymer (B) may be used alone or as a blend of two or more polymers which differ in compositional ratio, Mw, and/or Mw/Mn. The base polymer (B) may contain a hydrogenated ring-opening metathesis polymer in addition to the polymer, and as the hydrogenated ring-opening metathesis polymer, a polymer described in JP-A 2003-66612 can be used.

### (C) Organic Solvent

The chemically amplified resist composition may comprise an organic solvent as component (C). The organic solvent (C) is not particularly limited as long as it can dissolve each component described above and each component described below. Specific examples of the organic solvent include ketones such as cyclopentanone, cyclohexanone, and methyl-2-n-pentyl ketone; alcohols such as 3-methoxybutanol, 3 methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); and mixtures thereof.

Of these organic solvents, 1-ethoxy-2-propanol, PGMEA, cyclohexanone, GBL, DAA, and mixtures thereof are preferred because the base polymer as component (B) is particularly soluble therein.

In the chemically amplified resist composition, the organic solvent (C) is preferably added in an amount of 200 to 5,000 parts by weight, more preferably 400 to 3,500 parts by weight per 80 parts by weight of the base polymer (B). The organic solvent (C) may be used alone or in admixture.

### (D) Quencher

The chemically amplified resist composition may comprise a quencher as component (D). In the invention, the quencher is a material for forming a desired pattern by trapping an acid generated from a photoacid generator in a chemically amplified resist composition to prevent diffusion to an unexposed region.

Examples of the quencher (D) include an onium salt having the formula (2) or (3).

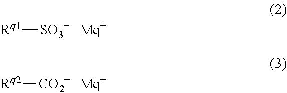

In formula (2), R⁹¹ is a hydrogen atom or a C₁-C₄₀ hydrocarbyl group which may contain a heteroatom, exclusive of a hydrocarbyl group in which a hydrogen atom bonded to a carbon atom at α-position of a sulfo group is substituted with a fluorine atom or a fluoroalkyl group. In formula (3), R⁹² is a hydrogen atom or a C₁-C₄₀ hydrocarbyl group which may contain a heteroatom.

Specific examples of the C₁-C₄₀ hydrocarbyl group R⁹¹ include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.02,6]decyl, and adamantyl; C₆-C₄₀ aryl groups such as phenyl, naphthyl and anthracenyl; and combinations thereof. In the hydrocarbyl group, some or all of the hydrogen atoms may be substituted by a moiety containing a heteroatom such as an oxygen atom, a sulfur atom, a nitrogen atom, or a halogen atom, and some constituent —CH₂—may be replaced by a moiety containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

Specific examples of the hydrocarbyl group R⁹² include, in addition to the substituents exemplified as specific examples of R⁹¹, fluorinated saturated hydrocarbyl groups such as trifluoromethyl and trifluoroethyl, and fluorinated aryl groups such as pentafluorophenyl and 4-trifluoromethylphenyl.

Specific examples of the anion of the onium salt having formula (2) are shown below, but not limited thereto.

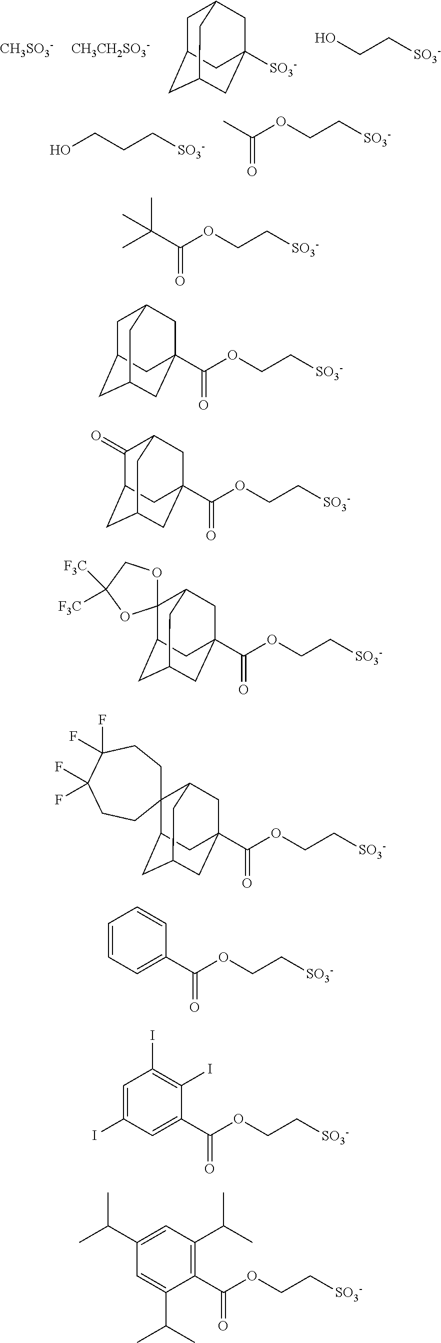

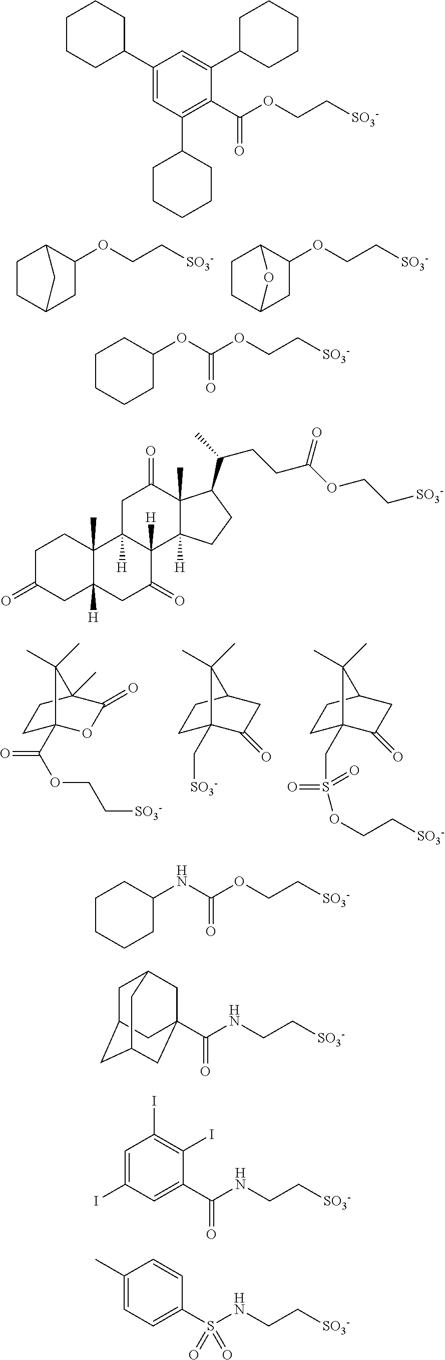

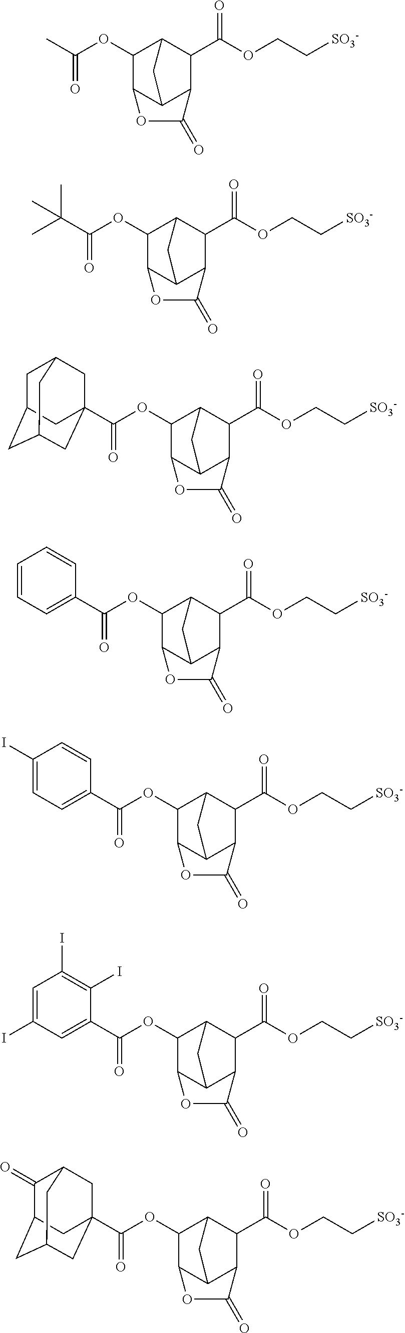

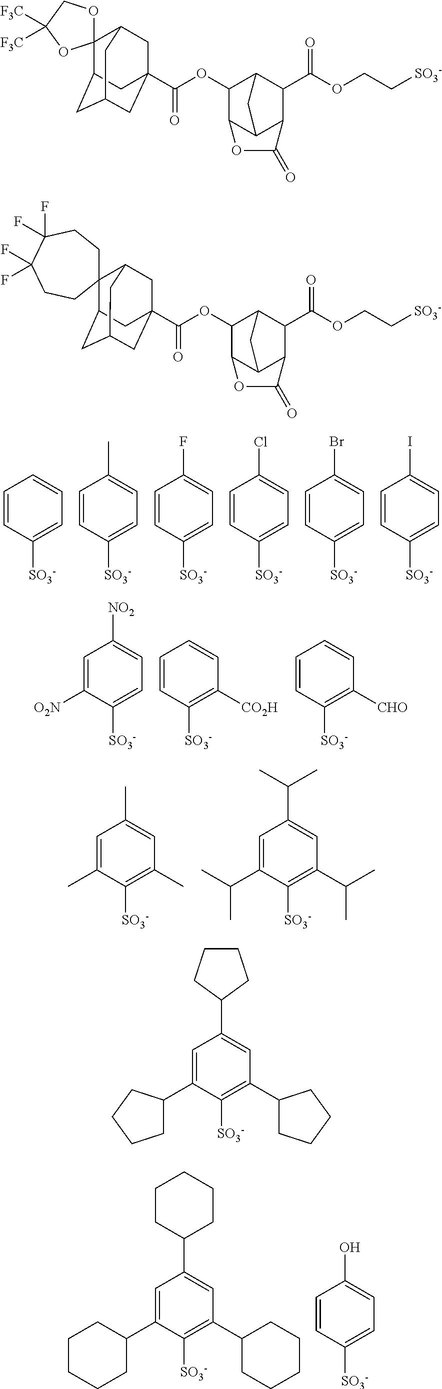

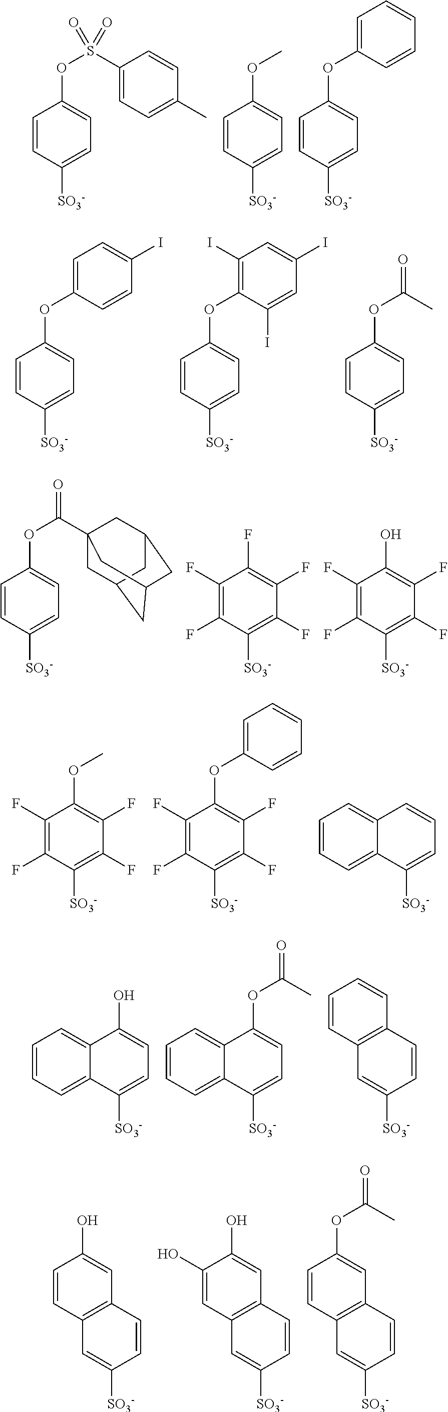

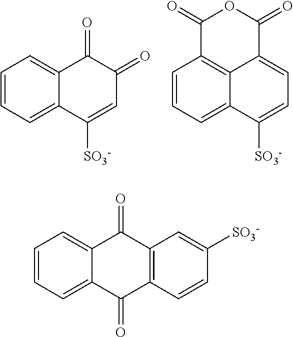

Specific examples of the anion of the onium salt having formula (3) are shown below, but not limited thereto.

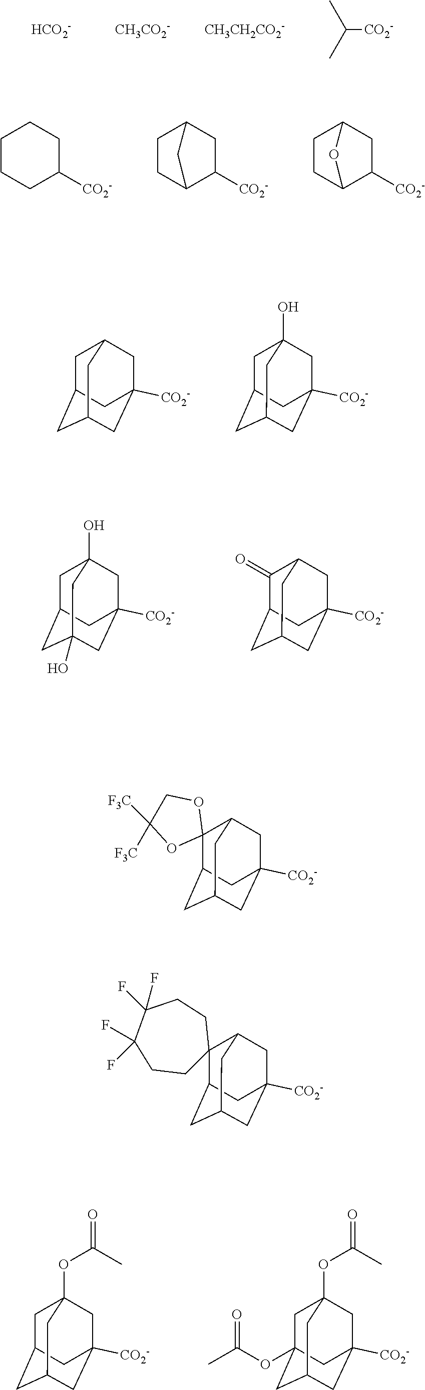

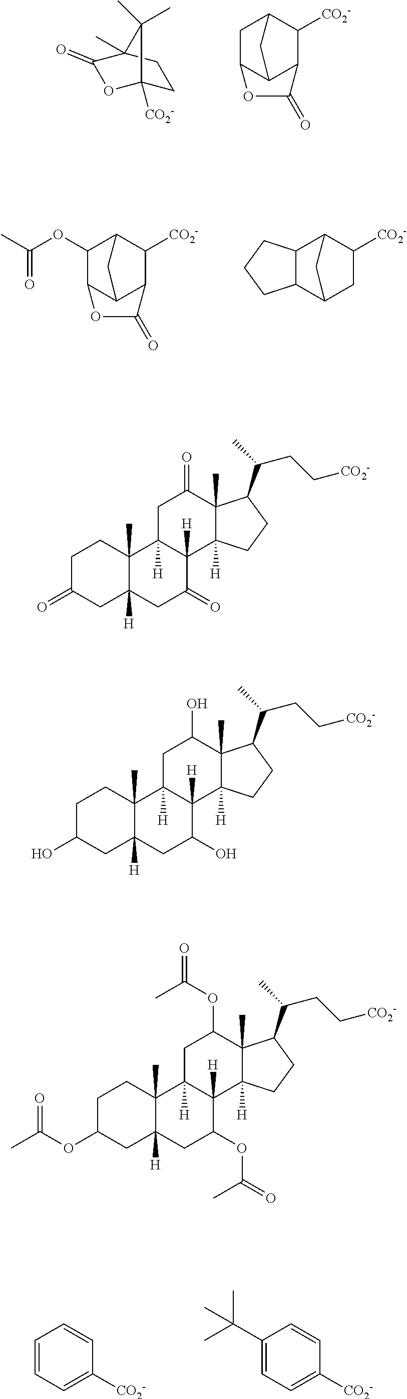

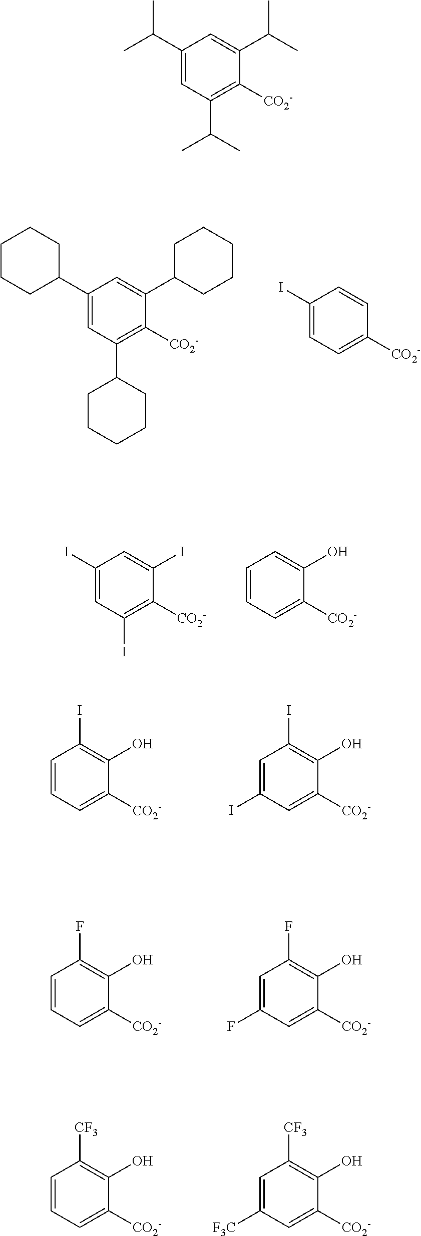

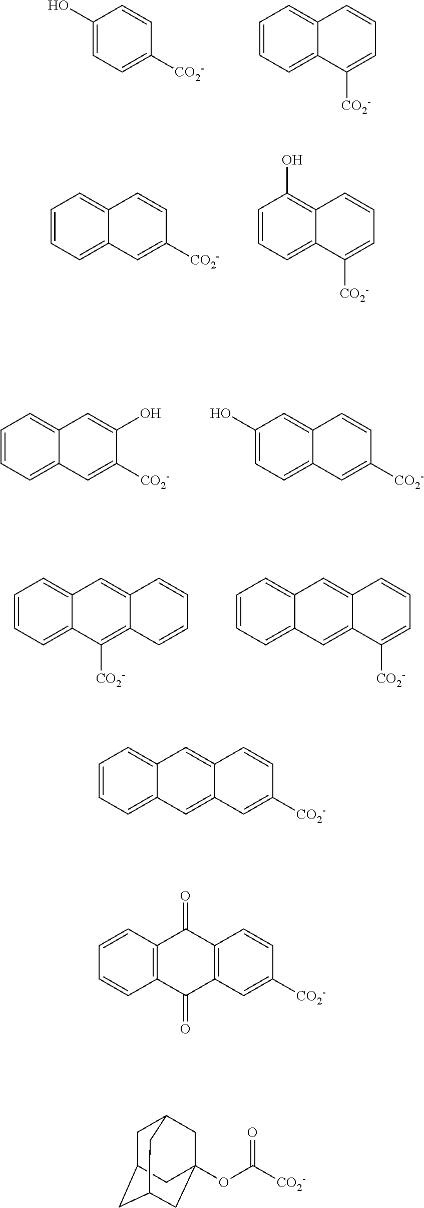

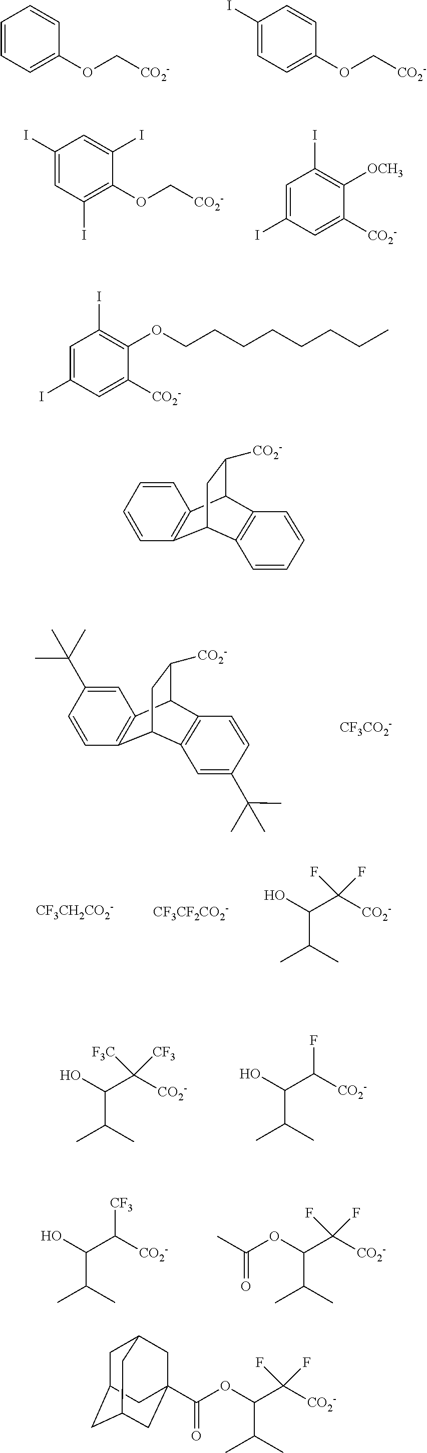

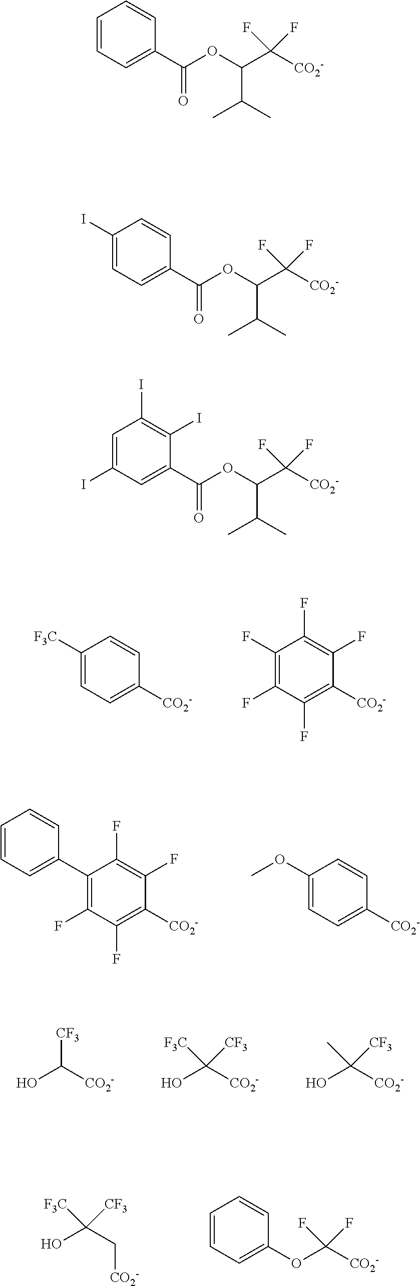

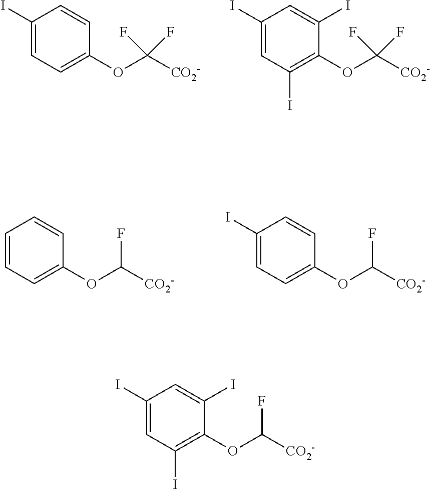

In formulae (2) and (3), Mq⁺ is an onium cation. Examples of the onium cation include a sulfonium cation, an iodonium cation, and an ammonium cation. Specific examples of the sulfonium cation and the iodonium cation include those exemplified as the specific examples of the sulfonium cation and the iodonium cation having Z⁺ in the description of formula (1), but not limited thereto. Specific examples of the ammonium cation include those having the formula (am-1).

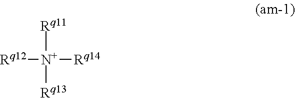

In formula (am-1), R^{q11} to R^{q14} are each independently a C₁-C₄₀ hydrocarbyl group which may contain a heteroatom. R^{q11} and R^{q12} may bond together to form a ring with the nitrogen atom to which they are attached. Specific examples of the hydrocarbyl group are as exemplified above for the hydrocarbyl group of R¹ in the description of formula (1A).

Specific examples of the ammonium cation having formula (am-1) are shown below, but not limited thereto.

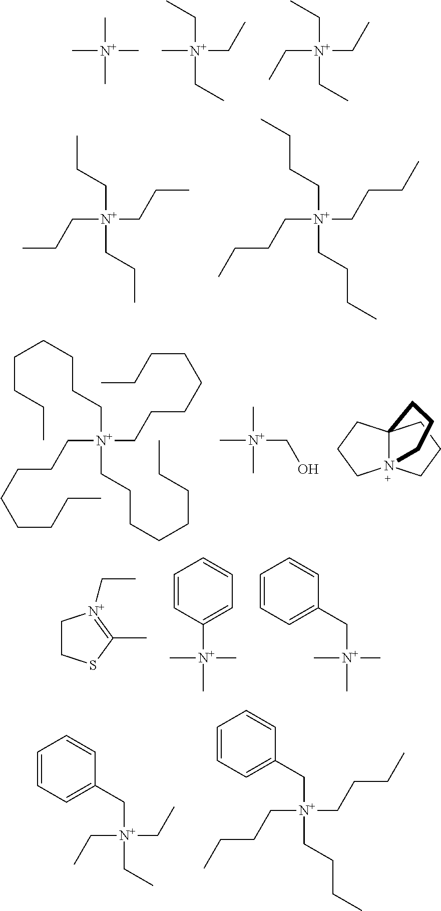

Specific examples of the onium salt having formula (2) or (3) include any combination of the foregoing anion and cation. These onium salts are easily prepared by an ion exchange reaction using a known organic chemical method. For the ion exchange reaction, reference may be made to JP-A 2007-145797, for example.

The onium salt having formula (2) or (3) functions as a quencher in the chemically amplified resist composition. This is because the counter anion of the onium salt is a conjugated base of a weak acid. The weak acid indicates an acidity insufficient to deprotect an acid labile group from an acid labile group-containing unit in the base polymer. The onium salt having formula (2) or (3) functions as a quencher when used in combination with an onium salt type photoacid generator having a conjugated base of a strong acid (typically a sulfonic acid which is fluorinated at α-position) as the counter anion. In a system using a mixture of an onium salt capable of generating a strong acid (e.g., α-position fluorinated sulfonic acid) and an onium salt capable of generating a weak acid (e.g., non-fluorinated sulfonic acid or carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

As the quencher (D), onium salts having a sulfonium cation and a phenoxide anion site in a common molecule as described in JP 6848776, onium salts having a sulfonium cation and a carboxylate anion site in a common molecule as described in JP 6583136 and JP-A 2020-200311, and onium salts having an iodonium cation and a carboxylate anion site in a common molecule as described in JP 6274755 can also be used.

If a photoacid generator capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the chemically amplified resist composition comprises the onium salt having formula (2) or (3) as the quencher (D), the amount of the onium salt is preferably 0.1 to 20 parts by weight, more preferably 0.1 to 10 parts by weight per 80 parts by weight of the base polymer (B). If the onium salt type quencher as component (D) is in the above range, the resolution is excellent, and the sensitivity is not significantly lowered, which is preferable. The onium salt having formula (2) or (3) can be used alone or in admixture.

The chemically amplified resist composition may comprise a nitrogen-containing compound as the quencher (D). Examples of the nitrogen-containing compound as component (D) include primary, secondary, and tertiary amine compounds, specifically amine compounds having a hydroxy group, an ether bond, an ester bond, a lactone ring, a cyano group, or a sulfonate ester bond, as described in paragraphs to of JP-A 2008-111103. Examples of the compounds include primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-46501, for example.

When the chemically amplified resist composition comprises a nitrogen-containing compound as the quencher (D), the amount of the nitrogen-containing compound is preferably 0.001 to 12 parts by weight, more preferably 0.01 to 8 parts by weight per 80 parts by weight of the base polymer (B). The nitrogen-containing compound may be used alone or in admixture.

### (E) Other Photoacid Generator

The chemically amplified resist composition may comprise a photoacid generator other than component (A)(hereinafter, the photoacid generator is also referred to as other photoacid generator) as component (E). The other photoacid generator is not particularly limited as long as it is a compound that generates an acid upon exposure to high-energy radiation. Examples of the preferred other photoacid generator include those having the formula (4) or (5).

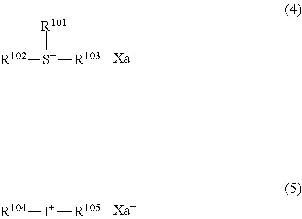

In formula (4), R¹⁰¹ to R¹⁰⁵ are each independently a C₁-C₂₀ hydrocarbyl group which may contain a heteroatom. Any two of R¹⁰¹, R¹⁰², and R¹⁰³ may bond together to form a ring with the sulfur atom to which they are attached.

Specific examples of the cation of the sulfonium salt having formula (4) and the cation of the iodonium salt having formula (5) are as exemplified above for the specific examples of the sulfonium cation and the iodonium cation having Z⁺ in the description of formula (1), but not limited thereto.

In formulae (4) and (5), Xa⁻ is an anion of a strong acid. Examples of the anion of the strong acid include those having any of formulae (c1-1) to (c1-5).

As the other photoacid generator as component (E), a photoacid generator having the formula (6) is also preferred.

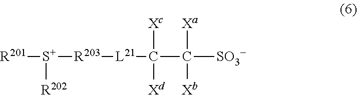

In formula (6), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may bond together to form a ring with the sulfur atom to which they are attached.

The C₁-C₃₀ hydrocarbyl groups R²⁰¹ and R²⁰² may be saturated or unsaturated and straight, branched, or cyclic. Specific examples thereof include C₁-C₃₀ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}] decyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In the hydrocarbyl group, some or all of the hydrogen atoms may be substituted by a moiety containing a heteroatom such as an oxygen atom, a sulfur atom, a nitrogen atom, or a halogen atom, and some constituent —CH₂—may be replaced by a moiety containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

The C₁-C₃₀ hydrocarbylene group R²⁰³ may be saturated or unsaturated and straight, branched, or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl, and adamantanediyl; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene. In the hydrocarbylene group, some or all of the hydrogen atoms may be substituted by a moiety containing a heteroatom such as an oxygen atom, a sulfur atom, a nitrogen atom, or a halogen atom, and some constituent —CH₂—may be replaced by a moiety containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group. The heteroatom is preferably an oxygen atom.

In formula (6), L²¹ is a single bond, an ether bond, or a C₁-C₂₀ hydrocarbylene group which may contain a heteroatom. The hydrocarbylene group may be saturated or unsaturated and straight, branched, or cyclic. Specific examples thereof are as exemplified above for the hydrocarbylene group R²⁰³.

In formula (6), X^{a}, X^{b}, X^{c}, and X^{d} are each independently a hydrogen atom, a fluorine atom, or a trifluoromethyl group. Provided that at least one of X^{a}, X^{b}, X^{c}, and X^{d} is a fluorine atom or a trifluoromethyl group.

As the photoacid generator having formula (6), a photoacid generator having the formula (6′) is preferred.

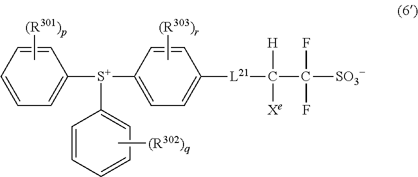

In formula (6′), L²¹ is as defined above. Xe is a hydrogen atom or a trifluoromethyl group, preferably a trifluoromethyl group. R³⁰¹, R³⁰², and R³⁰³ are each independently a hydrogen atom or a C₁-C₂₀ hydrocarbyl group which may contain a heteroatom. The hydrocarbyl group may be saturated or unsaturated and straight, branched, or cyclic. Specific examples thereof are as exemplified above for the hydrocarbyl group R^{fa1} in formula (c1-1-1). p and q are each independently 0, 1, 2, 3, 4, or 5, and r is 0, 1, 2, 3, or 4.

Examples of the photoacid generator having formula (6) are as exemplified above for the photoacid generator having formula (2) in JP-A 2017-26980.

Of the other photoacid generators, a photoacid generator containing an anion having the formula (c1-1-1) or (c1-4) has small acid diffusion and excellent solubility in solvents, and is particularly preferred. The photoacid generator having formula (6′) has extremely small acid diffusion, and is particularly preferred.

When the chemically amplified resist composition comprises other photoacid generator (E), the amount of the other photoacid generator is preferably 0.1 to 40 parts by weight, more preferably 0.5 to 20 parts by weight per 80 parts by weight of the base polymer (B). As long as the amount of the photoacid generator as component (E) is in the range, high resolution is achievable and the risk of foreign matter being formed after development or during stripping of resist film is avoided, which is preferable. The other photoacid generator (E) may be used alone or in admixture.

### (F) Surfactant

The chemically amplified resist composition may further comprise a surfactant as component (F). The surfactant (F) is preferably a surfactant which is insoluble or substantially insoluble in water and soluble in alkaline developer or a surfactant which is insoluble or substantially insoluble in water and alkaline developer. For the surfactant, reference should be made to those compounds described in JP-A 2010-215608 and JP-A 2011-16746.

While many examples of the surfactant which is insoluble or substantially insoluble in water and alkaline developer are described in the patent documents cited herein, preferred examples are surfactants FC-4430 (3M), Surflon® S-381 (AGC Seimi Chemical Co., Ltd.), Olfine® E1004 (Nissin Chemical Co., Ltd.), and KH-20 and KH-30 (AGC Seimi Chemical Co., Ltd.), and oxetane ring-opened polymers having the formula (surf-1).

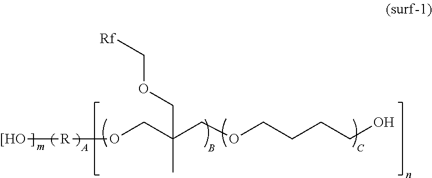

R, Rf, A, B, C, m, and n are applied to only formula (surf-1), independent of their descriptions other than for the surfactant. R is a di- to tetra-valent C₂-C₅ aliphatic group. Examples of the divalent aliphatic groups include ethylene, 1,4-butylene, 1,2-propylene, 2,2-dimethyl-1,3-propylene, and 1,5-pentylene. Examples of the tri- and tetra-valent groups are shown below.

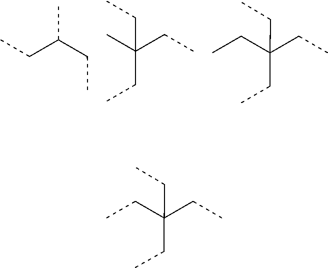

Herein the broken lines designate points of attachment, and are partial structures derived from glycerol, trimethylolethane, trimethylolpropane, and pentaerythritol, respectively.

Inter alia, a 1,4-butylene group, a 2,2-dimethyl-1,3-propylene group, and the like are preferred.

Rf is a trifluoromethyl group or a pentafluoroethyl group, preferably a trifluoromethyl group. m is an integer of 0 to 3, n is an integer of 1 to 4, and the sum of n and m is a valence of R and an integer of 2 to 4. A is 1. B is an integer of 2 to 25, preferably an integer of 4 to 20. C is an integer of 0 to 10, preferably 0 or 1. The formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

When a resist protective film is not used in ArF immersion lithography, a surfactant which is insoluble or substantially insoluble in water and soluble in alkaline developer has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during alkaline development following exposure and post-exposure bake (PEB), and thus forms few or no foreign matter which becomes defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Specific examples of the polymeric surfactant include those comprising repeat units of at least one type selected from the formulae (7A) to (7E).

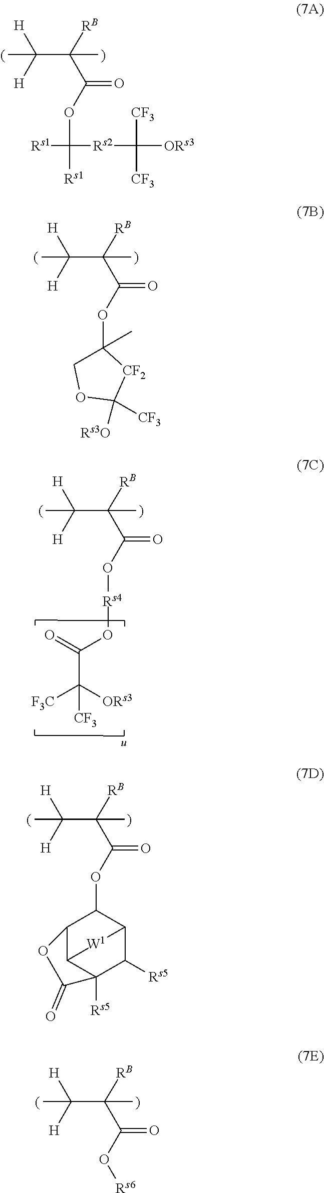

In formulae (7A) to (7E), R^{B} is a hydrogen atom, a fluorine atom, a methyl group, or a trifluoromethyl group. W¹ is —CH₂—, —CH₂CH₂—, —O—, or two separate-H. R^{s1} is each independently a hydrogen atom or a C₁-C₁₀ hydrocarbyl group. R^{s2} is a single bond or a C₁-C₅ straight or branched hydrocarbylene group. R^{s3} is each independently a hydrogen atom, a C₁-C₁₅ hydrocarbyl group, a fluorinated hydrocarbyl group, or an acid labile group. When R^{s3} is a hydrocarbyl group or a fluorinated hydrocarbyl group, an ether bond or a carbonyl group may intervene in a carbon-carbon bond. R^{s4} is a C₁-C₂₀ (u+1)-valent hydrocarbon group or a fluorinated hydrocarbon group. u is 1, 2, or 3. R^{s5} is each independently a hydrogen atom or a group having-C(═O)—O—R^{sa}. R^{sa} is a C₁-C₂₀ fluorinated hydrocarbyl group. R^{s6} is a C₁-C₁₅ hydrocarbyl group or a fluorinated hydrocarbyl group, and an ether bond or a carbonyl group may intervene in a carbon-carbon bond.

The C₁-C₁₀ hydrocarbyl group R^{s1} is preferably a saturated hydrocarbyl group, and may be straight, branched, or cyclic. Specific examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, and n-decyl; and C₃-C₁₀ cyclic saturated hydrocarbyl groups such as cyclopropyl, cyclobutyl, cyclopentyl, cyclohexyl, adamantyl, and norbornyl. Inter alia, C₁-C₆ hydrocarbyl groups are preferred.

The hydrocarbylene group R^{s2} is preferably a saturated hydrocarbylene group, and may be straight, branched, or cyclic. Examples thereof include a methylene group, an ethylene group, a propylene group, a butylene group, and a pentylene group.

The hydrocarbyl group R^{s3} or R^{s6} may be saturated or unsaturated and straight, branched, or cyclic. Specific examples thereof include a saturated hydrocarbyl group, and an aliphatic unsaturated hydrocarbyl group such as an alkenyl group or an alkynyl group, and the saturated hydrocarbyl group is preferred. Examples of the saturated hydrocarbyl group include an undecyl group, a dodecyl group, a tridecyl group, a tetradecyl group, and a pentadecyl group, in addition to those exemplified as the hydrocarbyl group R^{s1}. Examples of the fluorinated hydrocarbyl group R^{s3} or R^{s6} include the foregoing hydrocarbyl groups in which some or all carbon-bonded hydrogen atoms are substituted with fluorine atoms. In these groups, an ether bond or a carbonyl group may intervene in a carbon-carbon bond as mentioned above.

Specific examples of the acid labile group R^{s3} include groups of the above formulae (AL-3) to (AL-5), trialkylsilyl groups in which each alkyl group has 1 to 6 carbon atoms, and C₄-C₂₀ oxoalkyl groups.

The (u+1)-valent hydrocarbon or fluorinated hydrocarbon group R^{s4} may be straight, branched, or cyclic, and specific examples thereof include the foregoing hydrocarbyl or fluorinated hydrocarbyl groups from which u number of hydrogen atoms are eliminated.

The fluorinated hydrocarbyl group R^{sa} is preferably saturated, and may be straight, branched, or cyclic. Specific examples thereof include the foregoing hydrocarbyl groups in which some or all hydrogen atoms are substituted by fluorine atoms. Specific examples thereof include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Specific examples of the repeat units having formulae (7A) to (7E) are shown below, but not limited thereto. Herein R^{B} is as defined above.

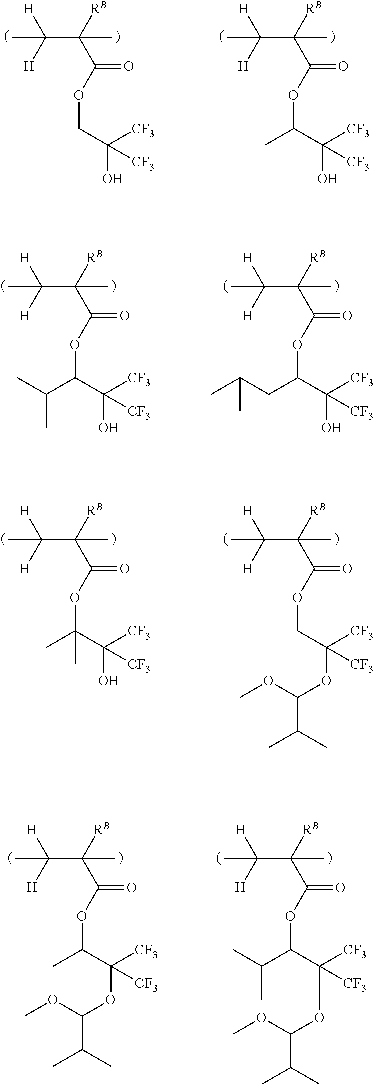

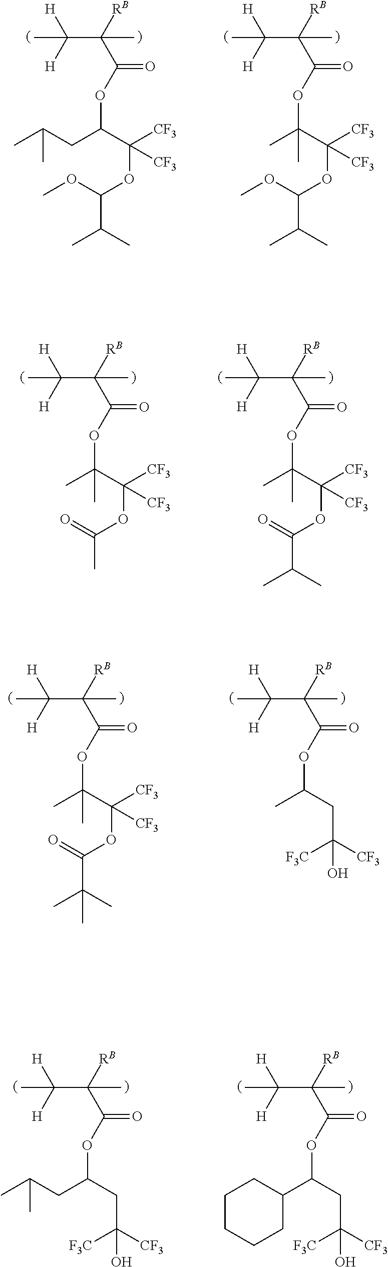

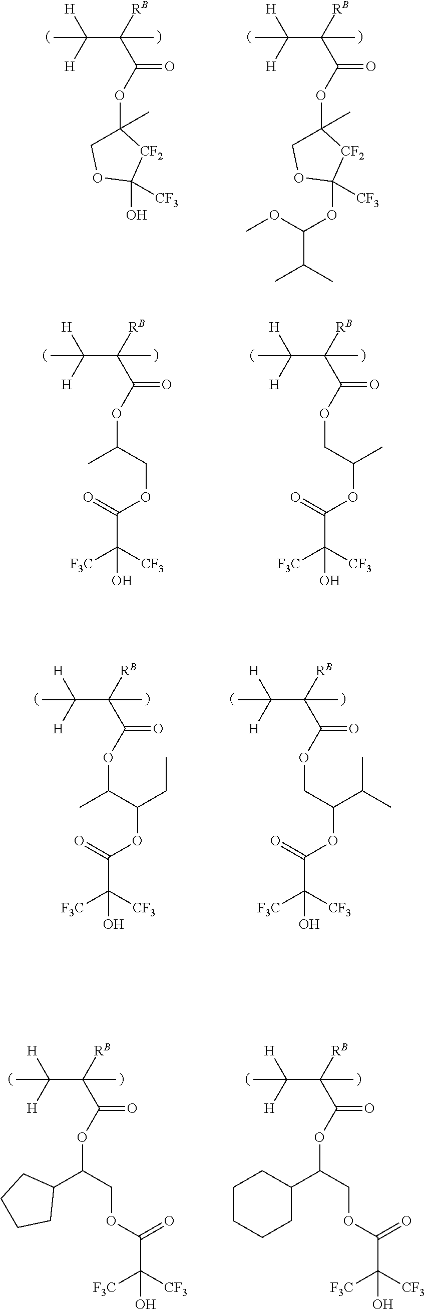

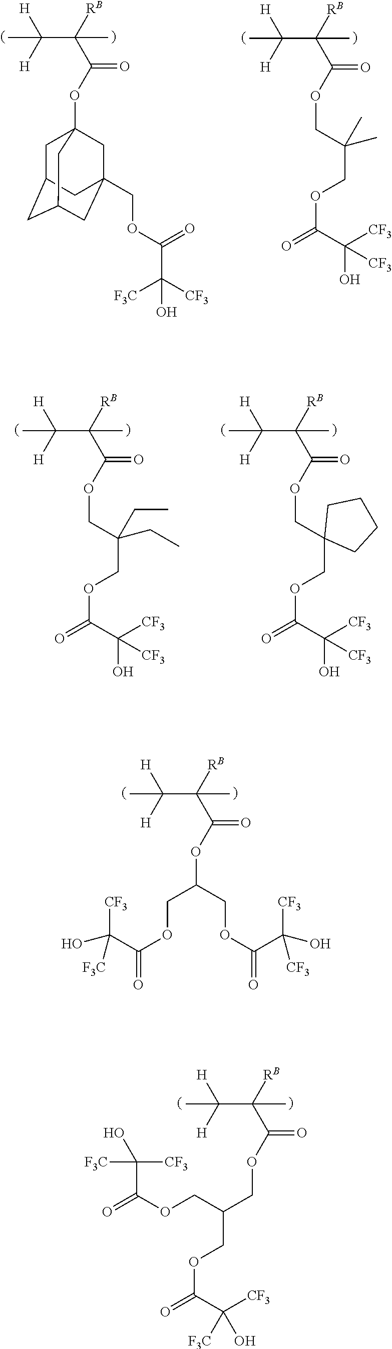

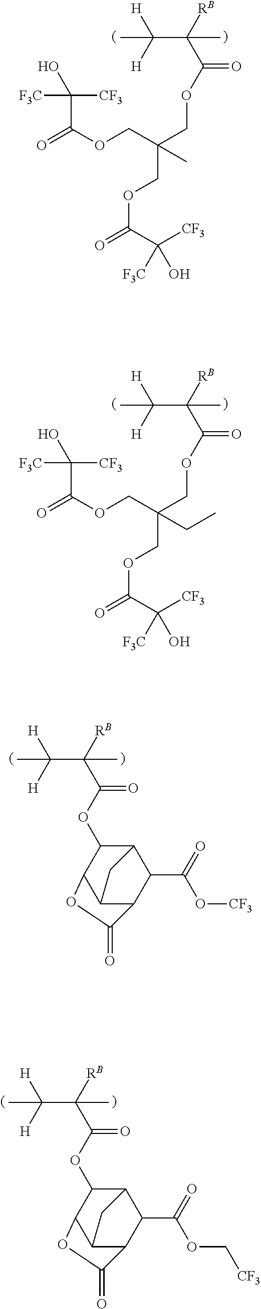

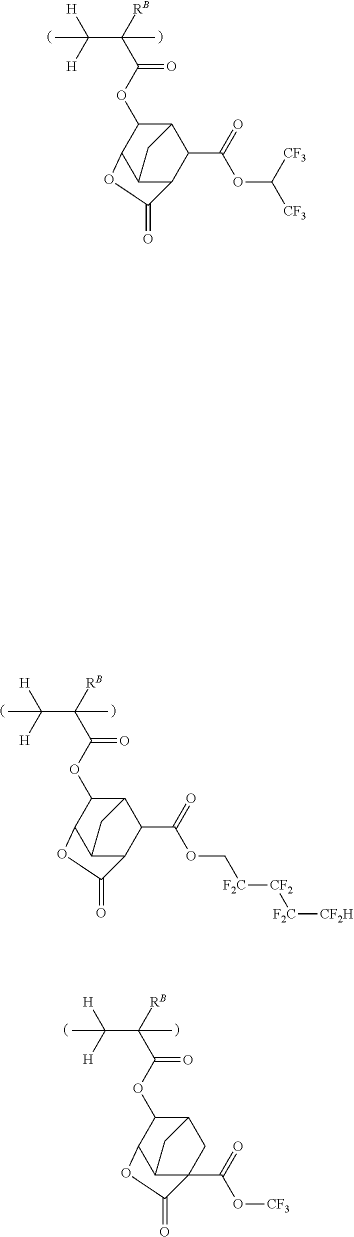

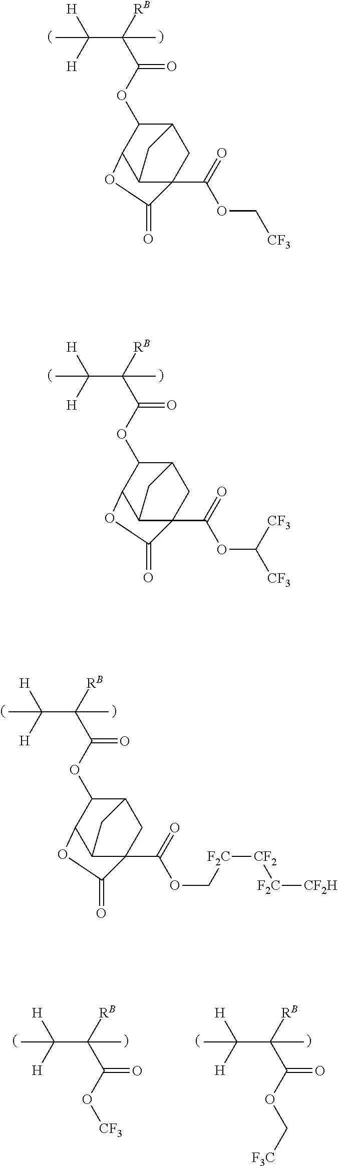

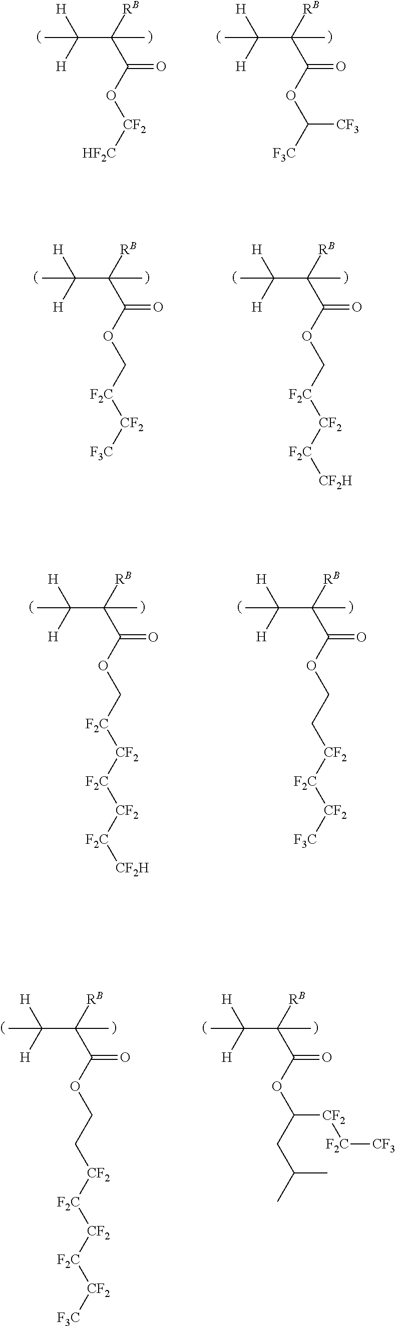

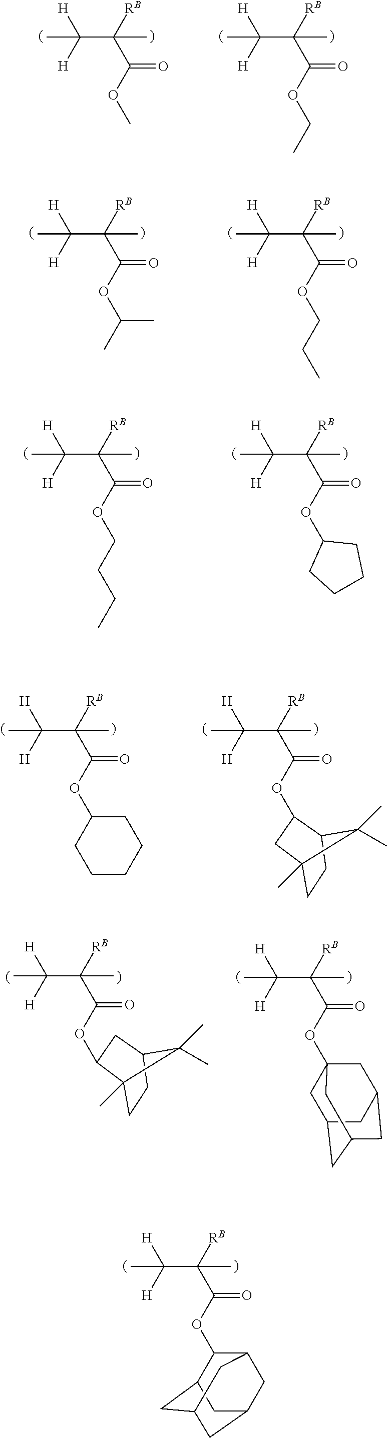

The polymeric surfactant may further contain repeat units other than the repeat units having formulae (7A) to (7E). Examples of the other repeat unit include repeat units derived from methacrylic acid and α-trifluoromethylacrylic acid derivatives. In the polymeric surfactant, the content of the repeat units having formulae (7A) to (7E) is preferably 20 mol % or more, more preferably 60 mol % or more, most preferably 100 mol % of the overall repeat units.

A Mw of the polymeric surfactant is preferably 1,000 to 500,000, more preferably 3,000 to 100,000. A Mw/Mn is preferably 1.0 to 2.0, more preferably 1.0 to 1.6.

Examples of a method of synthesizing the polymeric surfactant include a method in which a monomer containing a repeat unit having any one of formulae (7A) to (7E) and, if necessary, an unsaturated bond providing other repeat units is dissolved in an organic solvent, a radical initiator is added, and heating is performed for polymerization. Examples of the organic solvents used the polymerization include toluene, benzene, THE, diethyl ether, and dioxane. Examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or polymerization may be followed by protection or partial protection.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the chemically amplified resist composition comprises the surfactant (F), the amount of the surfactant is preferably 0.1 to 50 parts by weight, more preferably 0.5 to 10 parts by weight per 80 parts by weight of the base polymer (B). If the amount of the surfactant (F) is 0.1 parts by weight or more, the receding contact angle with water of the resist film at its surface is sufficiently improved, and if the amount of the surfactant (F) is 50 parts by weight or less, a rate of dissolution of the resist film surface in a developer is low, and the height of a small-size pattern formed therein is maintained. The surfactant (F) may be used alone or in admixture.

### (G) Other Components

The chemically amplified resist composition may further comprise (G) another component, for example, a compound which is decomposed with an acid to generate another acid (i.e., acid amplifier compound), an organic acid derivative, a fluorinated alcohol, and a compound having a Mw of 3,000 or less which changes its solubility in developer under the action of an acid (i.e., dissolution inhibitor). The acid amplifier compound is described in JP-A 2009-269953 and JP-A 2010-215608. The acid amplifier compound is preferably used in an amount of 0 to 5 parts, more preferably 0 to 3 parts by weight per 80 parts by weight of the base polymer (B). An extra amount of the acid amplifier in compound can make the acid diffusion control difficult and cause degradations to resolution and pattern profile. The organic acid derivative, the fluorinated alcohol, and the dissolution inhibitor are described in JP-A 2009-269953 and JP-A 2010-215608.

### Pattern Forming Process

The invention provides a pattern forming process comprising the steps of forming a resist film on a substrate using the chemically amplified resist composition, exposing the resist film to high-energy radiation, and developing the exposed resist film in a developer.

The substrate may be a substrate for integrated circuitry fabrication (Si, SiO₂, SiN, SiON, TiN, WSi, BPSG, SOG, organic antireflective film, etc.) or a substrate for mask circuitry fabrication (Cr, CrO, CrON, MoSi₂, SiO₂, etc.).

The resist film can be formed by, for example, applying the chemically amplified resist composition onto a substrate by a method such as spin coating so as to have a film thickness of preferably 0.05 to 2 μm, and prebaking the coating on a hot plate at preferably 60 to 150° C. for 1 to 10 minutes, more preferably 80 to 140° C. for 1 to 5 minutes.

Examples of the high-energy radiation used for exposure of the resist film include KrF excimer laser, ArF excimer laser, EB, and EUV of wavelength 3 to 15 nm. When KrF excimer laser, ArF excimer laser light, or EUV is used, the resist film is exposed through a mask having a desired pattern, preferably in a dose of 1 to 200 mJ/cm², more preferably 10 to 100 mJ/cm². When EB is used, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of 1.0 or more between the resist film and the projection lens may be employed if desired. In this case, it is also possible to use a protective film insoluble in water.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be subjected to PEB. PEB can be performed, for example, by heating the resist film on a hot plate at preferably 60 to 150° C. for 1 to 5 minutes, more preferably 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, the exposed region of the resist film is dissolved away, and a desired resist pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The developer for the organic solvent development is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvents may be used alone or in admixture.

### EXAMPLES

Synthesis Examples, Examples, and Comparative Examples are given below by way of illustration and not by way of limitation. The device used is as follows.
IR: NICOLET 6700 manufactured by Thermo Fisher Scientific Inc.
¹H-NMR: ECA-500 manufactured by JEOL Ltd.
MALDI TOF-MS: S3000 manufactured by JEOL Ltd.

### [1] Synthesis of Onium Salt

### Example 1-1

### Synthesis of PAG-1

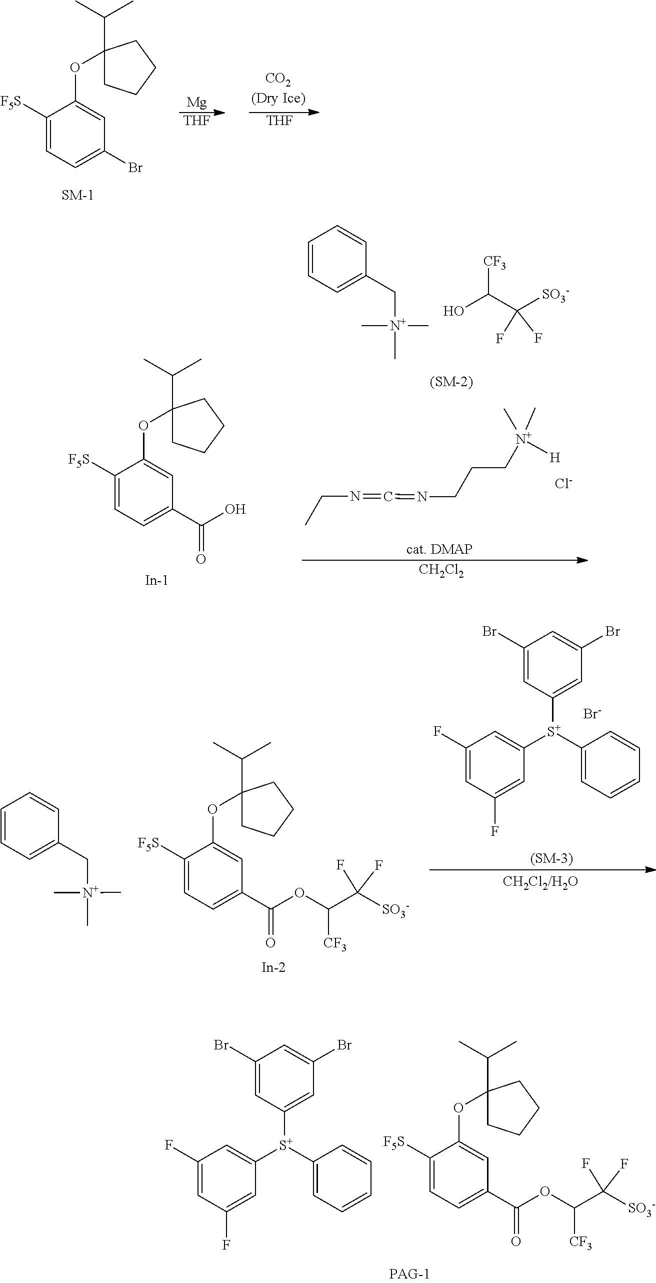

### (1) Synthesis of Intermediate In-1

A Grignard reagent was prepared using magnesium (1.3 g), reactant SM-1 (20.5 g), and THF (100 m1) under nitrogen atmosphere. Dry ice (50 g) was then suspended in THF (100 m1) and the prepared Grignard reagent was added. After addition, stirring was performed until the dry ice sublimated. After confirming sublimation of dry ice, 20 parts by weight of hydrochloric acid (11.0 g) was added to quench the reaction. The target product was extracted twice with ethyl acetate (100 m1), subjected to ordinary aqueous work-up, and recrystallized with hexane after distilling off the solvent to obtain 15.2 g of Intermediate In-1 as white crystals (yield 81%).

(2) Synthesis of Intermediate in-2

Under nitrogen atmosphere, Intermediate In-1 (15.2 g), reactant SM-2 (16.1 g), DMAP (0.5 g), and methylene chloride (80 g) were added and cooled in an ice bath. While maintaining the internal temperature at 20° C. or lower, 1-ethyl-3-(3-dimethylaminopropyl) carbodiimide hydrochloride (10.1 g) was added as powder. After addition, the reaction solution was heated to room temperature and aged for 12 hours. After aging, water was added to quench the reaction, ordinary aqueous work-up and solvent distillation were performed, and then, diisopropyl ether was added for recrystallization, thereby obtaining 27.4 g of Intermediate In-2 as white crystals (yield 92%).

### (3) Synthesis of PAG-1

Under nitrogen atmosphere, Intermediate In-2 (14.7 g), reactant SM-3 (11.8 g), methylene chloride (50 g), and water (30 g) were added, stirring was performed for 30 minutes, and then, the organic layer was separated, washed with water, and concentrated under reduced pressure. Diisopropyl ether was added to the concentrated solution, and recrystallization was performed to obtain 17.5 g of PAG-1 as a target product in the form of white crystals (yield 95%).

The TOF-MS results for PAG-1 are shown below.

### MALDI TOF-MS:

POSITIVE M⁺335 (corresponding to C₁₈H₁₁F₄S⁺)
NEGATIVE M⁻585 (corresponding to C₁₈H₁₉F₁₀O₆S₂⁻)

### Examples 1-2 to 1-10

### Synthesis of PAG-2 to PAG-10

Various onium salts were synthesized by various organic synthesis reactions with corresponding reactants. The structure of the onium salt used in the chemically amplified resist composition is shown below.

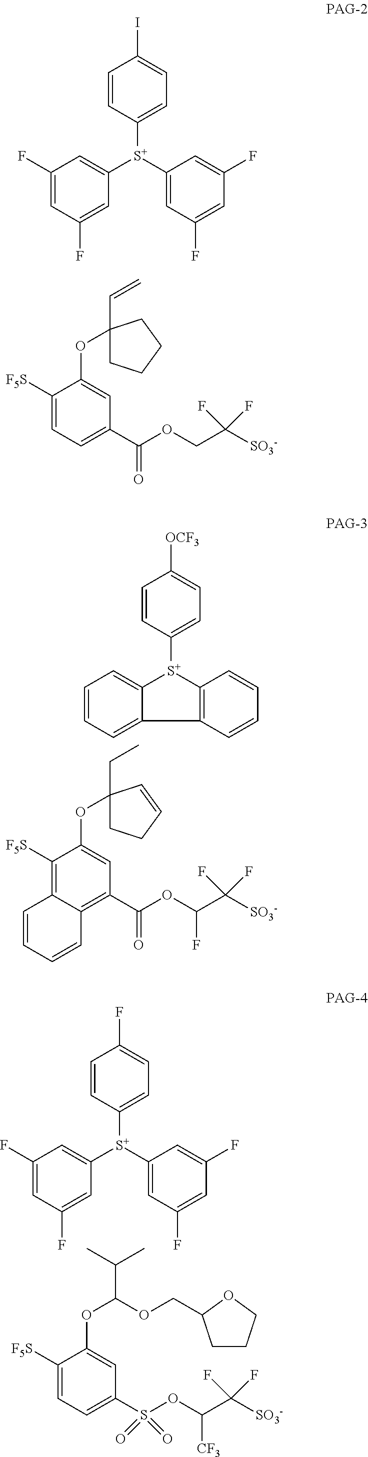

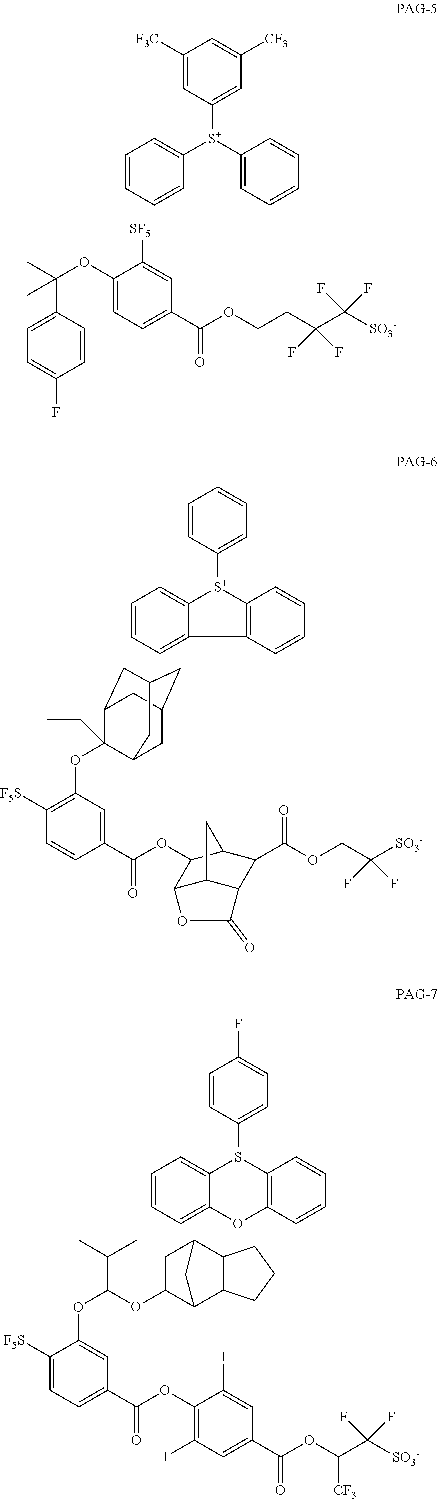

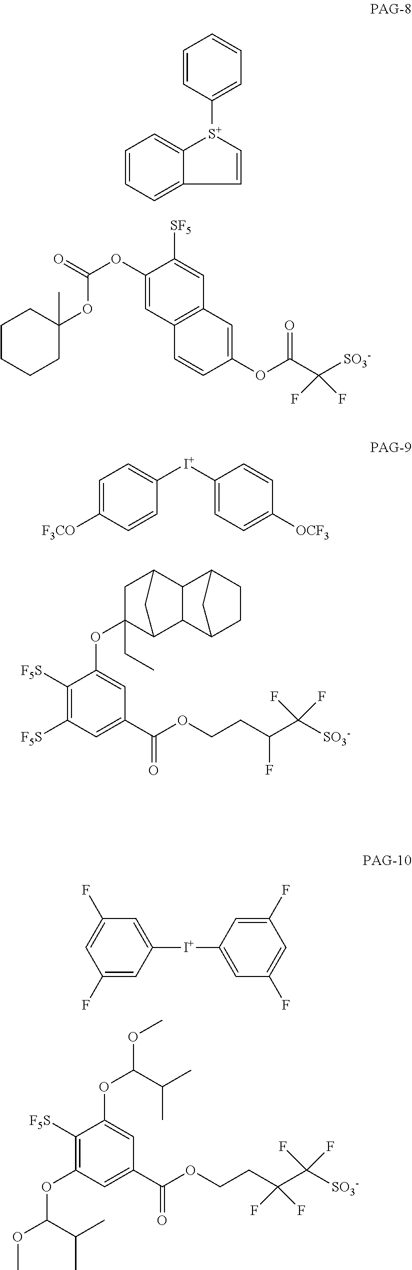

### [2] Synthesis of Base Polymer

### Synthesis Example

### Synthesis of Base Polymers (P-1 to P-5)

The respective monomers were combined to perform a copolymerization reaction in MEK as a solvent, the reaction solution was charged into hexane, and the precipitated solid was washed with hexane, isolated, and dried to obtain base polymers (P-1 to P-5) having the following compositions. The composition of the resulting base polymer was confirmed by 1H-NMR, and the Mw and Mw/Mn were confirmed by GPC (solvent: THF, standard: polystyrene).

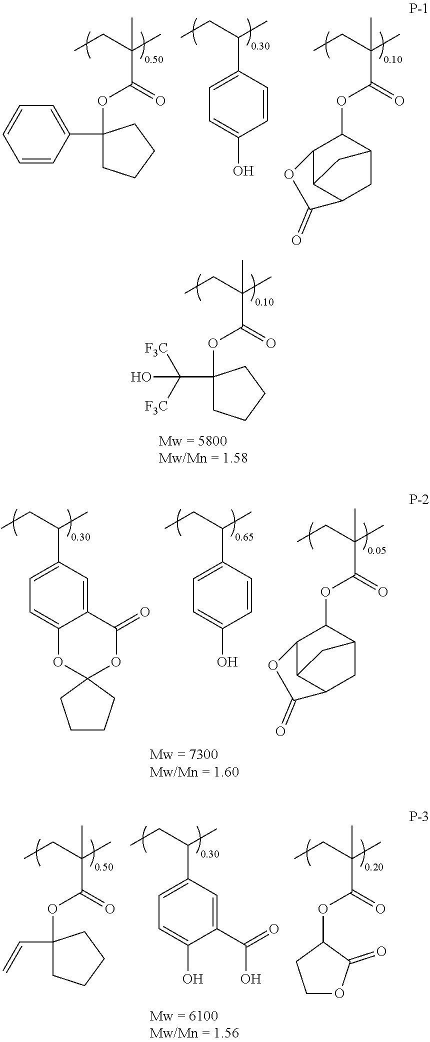

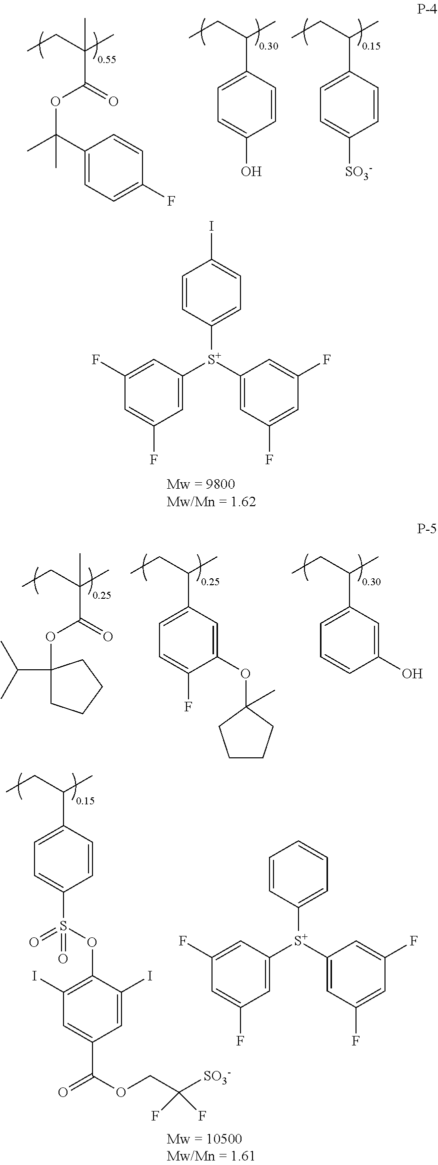

### [3] Preparation of Chemically Amplified Resist Composition

### Examples 2-1 to 2-32 and Comparative Examples 1-1 to 1-21

Chemically amplified resist compositions (R-1 to R-32, CR-1 to CR-21) were prepared by dissolving the sulfonium salt (PAG-1 to PAG-10), comparative photoacid generator (PAG-A to PAG-D), other photoacid generator (PAG-X, PAG-Y), base polymer (P-1 to P-5), and quencher (Q-1 to Q-4) in a solvent containing 0.01 wt % of surfactant A (Omnova Solutions, Inc.) in accordance with the formulation shown in Tables 1 and 2, and filtering through a Teflon® filter with a pore size of 0.2 μm.

TABLE 1

| | | | Base | Photoacid | | Other photoacid | | | |
|---|---|---|---|---|---|---|---|---|---|
| | | Resist | polymer | generator | Quencher | generator | Solvent 1 | Solvent 2 | Solvent 3 |
| | | composition | (pbw) | (pbw) | (pbw) | (pbw) | (pbw) | (pbw) | (pbw) |
| Example | 2-1 | R-1 | P-1 (80) | PAG-1 (24) | Q-1 (8.0) | — | PGMEA (2250) | EL (2800) | DAA (550) |
| | 2-2 | R-2 | P-1 (80) | PAG-2 (24) | Q-1 (8.2) | — | PGMEA (2250) | EL (2800) | DAA (550) |
| | 2-3 | R-3 | P-1 (80) | PAG-3 (24) | Q-1 (8.0) | — | PGMEA (2250) | EL (2800) | DAA (550) |
| | 2-4 | R-4 | P-1 (80) | PAG-4 (24) | Q-1 (7.8) | — | PGMEA (2250) | EL (2800) | DAA (550) |
| | 2-5 | R-5 | P-1 (80) | PAG-5 (24) | Q-1 (8.0) | — | PGMEA (2250) | EL (2800) | DAA (550) |
| | 2-6 | R-6 | P-1 (80) | PAG-6 (24) | Q-1 (8.0) | — | PGMEA (2250) | EL (2800) | DAA (550) |
| | 2-7 | R-7 | P-1 (80) | PAG-7 (24) | Q-1 (8.2) | — | PGMEA (2250) | EL (2800) | DAA (550) |
| | 2-8 | R-8 | P-1 (80) | PAG-8 (24) | Q-1 (8.0) | — | PGMEA (2250) | EL (2800) | DAA (550) |
| | 2-9 | R-9 | P-1 (80) | PAG-9 (24) | Q-1 (7.6) | — | PGMEA (2250) | EL (2800) | DAA (550) |
| | 2-10 | R-10 | P-1 (80) | PAG-10 (24) | Q-1 (8.0) | — | PGMEA (2250) | EL (2800) | DAA (550) |
| | 2-11 | R-11 | P-1 (80) | PAG-1 (24) | Q-2 (8.0) | — | PGMEA (2250) | EL (2800) | DAA (550) |
| | 2-12 | R-12 | P-1 (80) | PAG-1 (24) | Q-3 (8.2) | — | PGMEA (2250) | EL (2800) | DAA (550) |
| | 2-13 | R-13 | P-1 (80) | PAG-1 (24) | Q-1 (4.0) | — | PGMEA (2250) | EL (2800) | DAA (550) |
| | | | | | Q-4 (3.8) | | | | |
| | 2-14 | R-14 | P-1 (80) | PAG-1 (16) | Q-3 (8.0) | PAG-X (6) | PGMEA (2250) | EL (2800) | DAA (550) |
| | 2-15 | R-15 | P-1 (80) | PAG-2 (14) | Q-1 (8.0) | PAG-Y (8) | PGMEA (2250) | EL (2800) | DAA (550) |
| | 2-16 | R-16 | P-2 (80) | PAG-1 (24) | Q-1 (8.2) | — | PGMEA (2250) | EL (2800) | DAA (550) |
| | 2-17 | R-17 | P-2 (80) | PAG-2 (24) | Q-1 (8.0) | — | PGMEA (2250) | EL (2800) | DAA (550) |
| | 2-18 | R-18 | P-2 (80) | PAG-4 (24) | Q-3 (7.8) | — | PGMEA (2250) | EL (2800) | DAA (550) |
| | 2-19 | R-19 | P-2 (80) | PAG-6 (24) | Q-2 (8.0) | — | PGMEA (2250) | EL (2800) | DAA (550) |
| | 2-20 | R-20 | P-2 (80) | PAG-9 (12) | Q-1 (8.0) | PAG-Y (12) | PGMEA (2250) | EL (2800) | DAA (550) |
| | 2-21 | R-21 | P-3 (80) | PAG-1 (24) | Q-1 (7.8) | — | PGMEA (2250) | EL (2800) | DAA (550) |
| | 2-22 | R-22 | P-3 (80) | PAG-5 (24) | Q-3 (7.8) | — | PGMEA (2250) | EL (2800) | DAA (550) |
| | 2-23 | R-23 | P-3 (80) | PAG-7 (12) | Q-3 (4.0) | PAG-X (12) | PGMEA (2250) | EL (2800) | DAA (550) |
| | | | | | Q-4 (3.8) | | | | |
| | 2-24 | R-24 | P-3 (80) | PAG-10 (24) | Q-1 (8.2) | — | PGMEA (2250) | EL (2800) | DAA (550) |
| | 2-25 | R-25 | P-4 (80) | PAG-1 (24) | Q-3 (7.8) | — | PGMEA (2250) | EL (2800) | DAA (550) |
| | 2-26 | R-26 | P-4 (80) | PAG-3 (24) | Q-1 (8.0) | — | PGMEA (2250) | EL (2800) | DAA (550) |
| | 2-27 | R-27 | P-4 (80) | PAG-7 (24) | Q-2 (4.0) | — | PGMEA (2250) | EL (2800) | DAA (550) |
| | | | | | Q-4 (3.8) | | | | |
| | 2-28 | R-28 | P-4 (80) | PAG-9 (24) | Q-3 (7.8) | — | PGMEA (2250) | EL (2800) | DAA (550) |
| | 2-29 | R-29 | P-5 (80) | PAG-1 (24) | Q-1 (8.0) | — | PGMEA (2250) | EL (2800) | DAA (550) |
| | 2-30 | R-30 | P-5 (80) | PAG-2 (24) | Q-3 (7.8) | — | PGMEA (2250) | EL (2800) | DAA (550) |
| | 2-31 | R-31 | P-5 (80) | PAG-5 (24) | Q-1 (8.0) | — | PGMEA (2250) | EL (2800) | DAA (550) |
| | 2-32 | R-32 | P-5 (80) | PAG-10 (24) | Q-1 (8.0) | — | PGMEA (2250) | EL (2800) | DAA (550) |

TABLE 2

| | | | Base | Photoacid | | Other photoacid | | | |
|---|---|---|---|---|---|---|---|---|---|
| | | Resist | polymer | generator | Quencher | generator | Solvent 1 | Solvent 2 | Solvent 3 |
| | | composition | (pbw) | (pbw) | (pbw) | (pbw) | (pbw) | (pbw) | (pbw) |
| Comparative | 1-1 | CR-1 | P-1 (80) | PAG-A (24) | Q-1 (8.0) | — | PGMEA (2250) | EL (2800) | DAA (550) |
| Example | 1-2 | CR-2 | P-1 (80) | PAG-B (24) | Q-1 (8.2) | — | PGMEA (2250) | EL (2800) | DAA (550) |
| | 1-3 | CR-3 | P-1 (80) | PAG-C (24) | Q-1 (8.0) | — | PGMEA (2250) | EL (2800) | DAA (550) |
| | 1-4 | CR-4 | P-1 (80) | PAG-D (24) | Q-1 (7.8) | — | PGMEA (2250) | EL (2800) | DAA (550) |
| | 1-5 | CR-5 | P-1 (80) | PAG-A (24) | Q-2 (8.0) | — | PGMEA (2250) | EL (2800) | DAA (550) |
| | 1-6 | CR-6 | P-1 (80) | PAG-A (24) | Q-3 (8.2) | — | PGMEA (2250) | EL (2800) | DAA (550) |
| | 1-7 | CR-7 | P-1 (80) | PAG-A (24) | Q-1 (4.0) | — | PGMEA (2250) | EL (2800) | DAA (550) |
| | | | | | Q-4 (3.8) | | | | |
| | 1-8 | CR-8 | P-1 (80) | PAG-A (16) | Q-3 (8.0) | PAG-X (6) | PGMEA (2250) | EL (2800) | DAA (550) |
| | 1-9 | CR-9 | P-1 (80) | PAG-B (14) | Q-1 (8.0) | PAG-Y (8) | PGMEA (2250) | EL (2800) | DAA (550) |
| | 1-10 | CR-10 | P-2 (80) | PAG-A (24) | Q-1 (8.2) | — | PGMEA (2250) | EL (2800) | DAA (550) |
| | 1-11 | CR-11 | P-2 (80) | PAG-B (24) | Q-2 (8.0) | — | PGMEA (2250) | EL (2800) | DAA (550) |
| | 1-12 | CR-12 | P-2 (80) | PAG-C (12) | Q-1 (8.0) | PAG-Y (12) | PGMEA (2250) | EL (2800) | DAA (550) |
| | 1-13 | CR-13 | P-3 (80) | PAG-A (24) | Q-1 (7.8) | — | PGMEA (2250) | EL (2800) | DAA (550) |
| | 1-14 | CR-14 | P-3 (80) | PAG-C (24) | Q-3 (7.8) | — | PGMEA (2250) | EL (2800) | DAA (550) |
| | 1-15 | CR-15 | P-3 (80) | PAG-D (12) | Q-3 (4.0) | PAG-X (12) | PGMEA (2250) | EL (2800) | DAA (550) |
| | | | | | Q-4 (3.8) | | | | |
| | 1-16 | CR-16 | P-4 (80) | PAG-A (24) | Q-3 (7.8) | — | PGMEA (2250) | EL (2800) | DAA (550) |
| | 1-17 | CR-17 | P-4 (80) | PAG-B (24) | Q-2 (4.0) | — | PGMEA (2250) | EL (2800) | DAA (550) |
| | | | | | Q-4 (3.8) | | | | |
| | 1-18 | CR-18 | P-4 (80) | PAG-C (24) | Q-3 (7.8) | — | PGMEA (2250) | EL (2800) | DAA (550) |
| | 1-19 | CR-19 | P-5 (80) | PAG-A (24) | Q-1 (8.0) | — | PGMEA (2250) | EL (2800) | DAA (550) |
| | 1-20 | CR-20 | P-5 (80) | PAG-C (24) | Q-3 (7.8) | — | PGMEA (2250) | EL (2800) | DAA (550) |
| | 1-21 | CR-21 | P-5 (80) | PAG-D (24) | Q-1 (8.0) | — | PGMEA (2250) | EL (2800) | DAA (550) |

The solvents, other photoacid generators PAG-X and PAG-Y, comparative photoacid generators PAG-A to PAG-D, other photoacid generators PAG-X and PAG-Y, and quenchers Q-1 to Q-4 in Tables 1 and 2 are identified below.

### Solvent:

PGMEA (propylene glycol monomethyl ether acetate)
EL (ethyl lactate)
DAA (diacetone alcohol)

### Other Photoacid Generator: PAG-X and PAG-Y

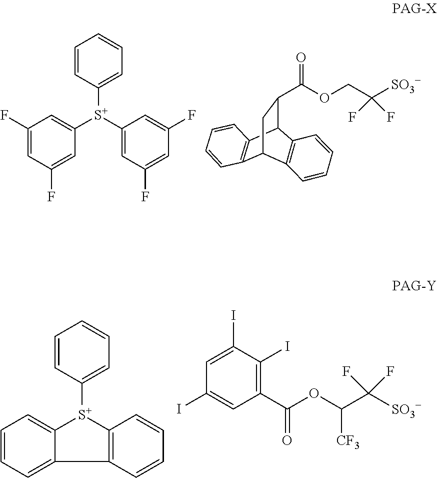

### Comparative Photoacid Generator: PAG-a to PAG-D

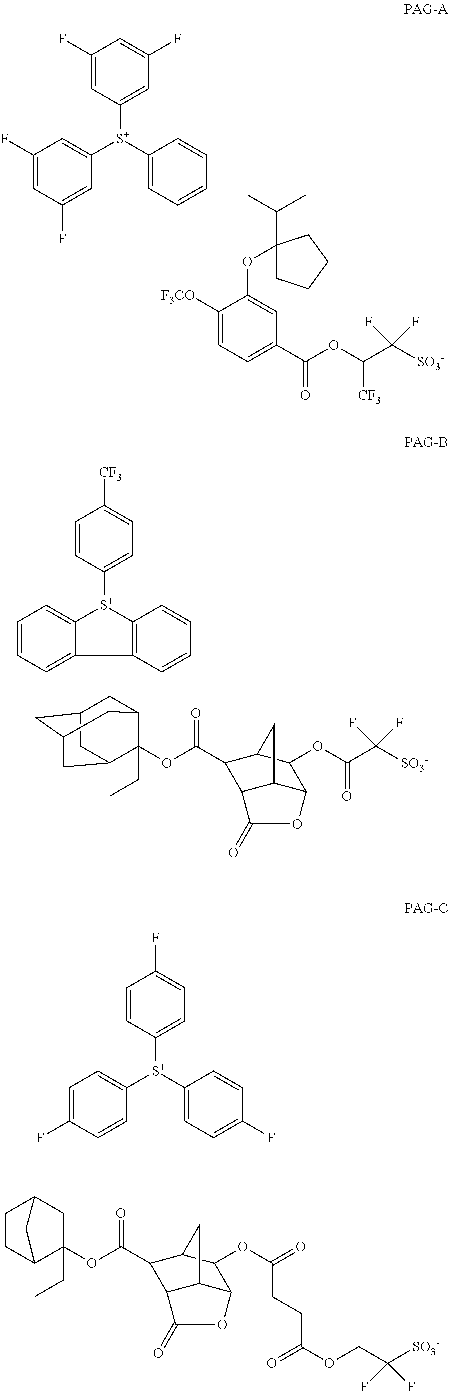

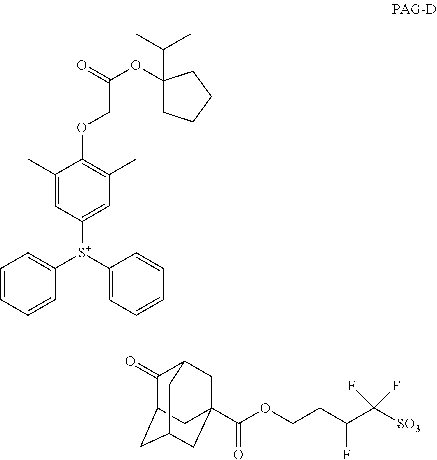

### Quencher: Q-1 to Q-4

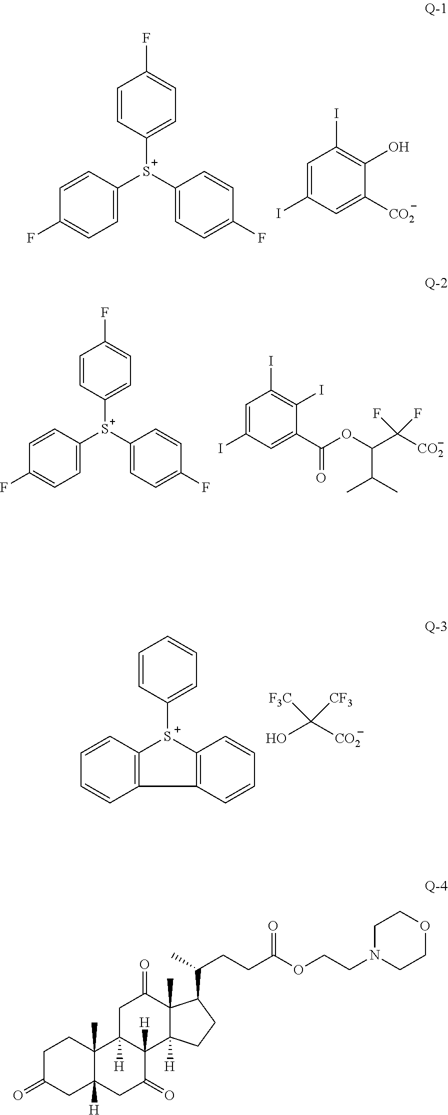

### [4] EUV Lithography Test (1)

### Examples 3-1 to 3-32 and Comparative Examples 2-1 to 2-21

Each of the chemically amplified resist compositions (R-1 to R-32, CR-1 to CR-21) shown in Tables 1 and 2 was spin coated on a Si substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 (ASML, NA 0.33, σ0.9/0.6, dipole illumination), the resist film was subjected to exposure with a line-and-space (LS) pattern having a size of 18 nm and a pitch of 36 nm (on-wafer size) while varying the dose and focus (dose pitch: 1 mJ/cm², focus pitch: 0.020 μm). After exposure, the resist film was baked (PEB) at the temperature shown in Tables 3 and 4 for 60 seconds. The resist film was puddle developed in a 2.38 wt % TMAH aqueous solution for 30 seconds, rinsed with a rinse fluid containing surfactant, and spin dried to form a positive pattern.

The LS pattern was observed under CD-SEM (CG6300, Hitachi High Technologies Corp.), whereupon sensitivity, EL, LWR, depth of focus (DOF), and collapse limit were evaluated according to the following methods. The results are shown in Tables 3 and 4.

### [Evaluation of Sensitivity]

The optimum dose Eop (mJ/cm²) which provided an LS pattern with a line width of 18 nm and a pitch of 36 nm was determined as an index of sensitivity. A smaller value indicates a higher sensitivity.

### [Evaluation of EL]

The exposure dose which provided an LS pattern with a space width of 18 nm±10% (i.e., 16.2 to 19.8 nm) was determined. EL (%) is calculated from the exposure doses according to the following equation. A greater value indicates better performance.

$$EL ( \% ) = ( \vert {E}_{1} - {E}_{2} \vert / Eop ) \times 100$$

Herein E1 is an optimum exposure dose which provides an LS pattern with a line width of 16.2 nm and a pitch of 36 nm,

E₂ is an optimum exposure dose which provides an LS pattern with a line width of 19.8 nm and a pitch of 36 nm, and

Eop is an optimum exposure dose which provides an LS pattern with a line width of 18 nm and a pitch of 36 nm.

### [Evaluation of LWR]

For the LS pattern formed by exposure at the optimum dose Eop, the line width was measured at 10 longitudinally spaced apart points, from which a 3-fold value (3σ) of the standard deviation (σ) was determined and reported as LWR. A smaller value of 3σ indicates a pattern having small roughness and uniform line width.

### [Evaluation of DOF]

As an index of depth of focus, a range of focus which provided an LS pattern with a size of 18 nm±10% (i.e., 16.2 to 19.8 nm) was determined. A greater value indicates a wider depth of focus.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 3

| | | | | | | | | Col- |
|---|---|---|---|---|---|---|---|---|
| | | Resist | PEB | Eop | | | | lapse |
| | | compo- | temp. | (mJ/ | EL | LWR | DOF | limit |
| | | sition | (° C.) | cm²) | (%) | (nm) | (nm) | (nm) |
| Example | 3-1 | R-1 | 95 | 33 | 18 | 2.3 | 110 | 10.5 |
| | 3-2 | R-2 | 100 | 32 | 19 | 2.2 | 120 | 10.9 |
| | 3-3 | R-3 | 100 | 34 | 17 | 2.3 | 100 | 10.8 |
| | 3-4 | R-4 | 95 | 3 | 18 | 2.4 | 110 | 10.9 |
| | 3-5 | R-5 | 105 | 32 | 17 | 2.4 | 110 | 11.1 |
| | 3-6 | R-6 | 100 | 33 | 18 | 2.3 | 100 | 11.2 |
| | 3-7 | R-7 | 95 | 34 | 18 | 2.3 | 120 | 10.8 |
| | 3-8 | R-8 | 95 | 35 | 17 | 2.3 | 110 | 10.9 |
| | 3-9 | R-9 | 100 | 32 | 16 | 2.2 | 110 | 11.0 |
| | 3-10 | R-10 | 100 | 33 | 19 | 2.5 | 120 | 11.2 |
| | 3-11 | R-11 | 100 | 34 | 17 | 2.3 | 110 | 11.1 |
| | 3-12 | R-12 | 95 | 32 | 18 | 2.5 | 100 | 11.2 |
| | 3-13 | R-13 | 105 | 33 | 17 | 2.3 | 110 | 10.7 |
| | 3-14 | R-14 | 100 | 32 | 18 | 2.3 | 120 | 11.1 |
| | 3-15 | R-15 | 95 | 33 | 18 | 2.2 | 110 | 10.6 |
| | 3-16 | R-16 | 95 | 34 | 17 | 2.2 | 120 | 10.9 |
| | 3-17 | R-17 | 100 | 32 | 19 | 2.4 | 120 | 11.2 |
| | 3-18 | R-18 | 95 | 35 | 16 | 2.5 | 110 | 11.3 |
| | 3-19 | R-19 | 95 | 34 | 17 | 2.4 | 110 | 11.1 |
| | 3-20 | R-20 | 100 | 32 | 18 | 2.5 | 120 | 10.8 |
| | 3-21 | R-21 | 100 | 33 | 17 | 2.3 | 110 | 10.9 |
| | 3-22 | R-22 | 100 | 35 | 19 | 2.2 | 120 | 11.1 |
| | 3-23 | R-23 | 95 | 34 | 18 | 2.5 | 100 | 11.2 |
| | 3-24 | R-24 | 95 | 33 | 18 | 2.4 | 120 | 10.7 |
| | 3-25 | R-25 | 100 | 34 | 17 | 2.3 | 100 | 11.0 |
| | 3-26 | R-26 | 100 | 32 | 17 | 2.4 | 110 | 10.9 |
| | 3-27 | R-27 | 100 | 33 | 18 | 2.5 | 120 | 11.1 |
| | 3-28 | R-28 | 95 | 32 | 17 | 2.3 | 110 | 11.4 |
| | 3-29 | R-29 | 95 | 34 | 19 | 2.5 | 100 | 11.1 |
| | 3-30 | R-30 | 100 | 33 | 18 | 2.4 | 120 | 10.8 |
| | 3-31 | R-31 | 100 | 35 | 18 | 2.4 | 110 | 10.9 |
| | 3-32 | R-32 | 100 | 33 | 18 | 2.3 | 110 | 10.7 |

TABLE 4

| | | | | | | | | Col- |
|---|---|---|---|---|---|---|---|---|
| | | Resist | PEB | Eop | | | | lapse |
| | | compo- | temp. | (mJ/ | EL | LWR | DOF | limit |
| | | sition | (° C.) | cm²) | (%) | (nm) | (nm) | (nm) |
| Compar- | 2-1 | CR-1 | 95 | 36 | 14 | 2.8 | 100 | 11.9 |
| ative | 2-2 | CR-2 | 100 | 37 | 13 | 2.9 | 90 | 12 |
| Example | 2-3 | CR-3 | 100 | 36 | 14 | 3 | 80 | 12.3 |
| | 2-4 | CR-4 | 100 | 37 | 13 | 3.1 | 80 | 12.4 |
| | 2-5 | CR-5 | 95 | 36 | 15 | 2.9 | 90 | 11.8 |
| | 2-6 | CR-6 | 100 | 35 | 14 | 2.8 | 80 | 11.9 |
| | 2-7 | CR-7 | 100 | 37 | 15 | 2.7 | 80 | 11.7 |
| | 2-8 | CR-8 | 100 | 38 | 14 | 2.9 | 90 | 11.6 |
| | 2-9 | CR-9 | 95 | 36 | 13 | 3.1 | 80 | 12 |
| | 2-10 | CR-10 | 100 | 37 | 12 | 2.7 | 70 | 12.1 |
| | 2-11 | CR-11 | 100 | 36 | 15 | 2.8 | 80 | 11.7 |
| | 2-12 | CR-12 | 100 | 37 | 14 | 2.8 | 90 | 11.9 |
| | 2-13 | CR-13 | 95 | 35 | 13 | 2.9 | 80 | 11.6 |
| | 2-14 | CR-14 | 105 | 36 | 14 | 3.1 | 70 | 12.1 |
| | 2-15 | CR-15 | 100 | 37 | 15 | 2.8 | 90 | 11.6 |
| | 2-16 | CR-16 | 95 | 37 | 14 | 2.7 | 90 | 11.5 |
| | 2-17 | CR-17 | 95 | 36 | 13 | 2.9 | 80 | 12.2 |
| | 2-18 | CR-18 | 100 | 37 | 14 | 2.8 | 100 | 12.1 |
| | 2-19 | CR-19 | 95 | 36 | 14 | 2.7 | 90 | 11.8 |
| | 2-20 | CR-20 | 95 | 37 | 13 | 3.1 | 80 | 11.6 |
| | 2-21 | CR-21 | 100 | 37 | 14 | 2.8 | 70 | 11.8 |

It is demonstrated in Tables 3 and 4 that chemically amplified resist compositions comprising onium salts within the scope of the invention exhibit a high sensitivity and improved values of EL, LWR, and DOF. Small values of collapse limit attest that in forming a small-size pattern, the pattern is resistant to collapse. The chemically amplified resist compositions are useful in the EUV lithography process.

### [5] EUV Lithography Test (2)

### Examples 4-1 to 4-32 and Comparative Examples 3-1 to 3-21

Each of the chemically amplified resist compositions (R-1 to R-32, CR-1 to CR-21) shown in Tables 1 and 2 was spin coated on a Si substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, o 0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 5 and 6 for 60 seconds and developed in a 2.38 wt % TMAH aqueous solution for 30 seconds to form a hole pattern having a size of 23 nm.

The pattern as developed was observed under CD-SEM (CG6300, Hitachi High Technologies Corp.). The dose (mJ/cm²) at which a pattern with a hole size of 23 nm was printed was determined as an index of sensitivity. The size of 50 holes was measured, from which a 3-fold value (3σ) of the standard deviation (σ) was determined as CDU. The results are shown in Tables 5 and 6.

TABLE 5

| | | Resist | PEB temp. | Eop | CDU |
|---|---|---|---|---|---|
| | | composition | (° C.) | (mJ/cm²) | (nm) |
| Example | 4-1 | R-1 | 90 | 23 | 2.1 |
| | 4-2 | R-2 | 95 | 22 | 2.4 |
| | 4-3 | R-3 | 90 | 24 | 2.3 |
| | 4-4 | R-4 | 90 | 25 | 2.3 |
| | 4-5 | R-5 | 90 | 23 | 2.2 |
| | 4-6 | R-6 | 95 | 22 | 2.3 |
| | 4-7 | R-7 | 95 | 24 | 2.4 |
| | 4-8 | R-8 | 90 | 23 | 2.3 |
| | 4-9 | R-9 | 95 | 23 | 2.2 |
| | 4-10 | R-10 | 95 | 24 | 2.4 |
| | 4-11 | R-11 | 95 | 23 | 2.3 |
| | 4-12 | R-12 | 90 | 25 | 2.5 |
| | 4-13 | R-13 | 90 | 23 | 2.3 |
| | 4-14 | R-14 | 90 | 23 | 2.4 |
| | 4-15 | R-15 | 90 | 24 | 2.3 |
| | 4-16 | R-16 | 85 | 25 | 2.2 |
| | 4-17 | R-17 | 95 | 25 | 2.4 |
| | 4-18 | R-18 | 95 | 23 | 2.4 |
| | 4-19 | R-19 | 90 | 25 | 2.3 |
| | 4-20 | R-20 | 95 | 23 | 2.5 |
| | 4-21 | R-21 | 95 | 22 | 2.2 |
| | 4-22 | R-22 | 95 | 22 | 2.3 |
| | 4-23 | R-23 | 95 | 24 | 2.4 |
| | 4-24 | R-24 | 90 | 23 | 2.3 |
| | 4-25 | R-25 | 95 | 24 | 2.5 |
| | 4-26 | R-26 | 95 | 22 | 2.3 |
| | 4-27 | R-27 | 95 | 25 | 2.2 |
| | 4-28 | R-28 | 95 | 24 | 2.4 |
| | 4-29 | R-29 | 90 | 22 | 2.3 |
| | 4-30 | R-30 | 95 | 23 | 2.2 |
| | 4-31 | R-31 | 95 | 24 | 2.4 |
| | 4-32 | R-32 | 90 | 23 | 2.3 |

TABLE 6

| | | Resist | PEB temp. | Eop | CDU |
|---|---|---|---|---|---|
| | | composition | (° C.) | (mJ/cm²) | (nm) |
| Comparative | 3-1 | CR-1 | 90 | 27 | 2.8 |
| Example | 3-2 | CR-2 | 95 | 26 | 2.7 |
| | 3-3 | CR-3 | 95 | 28 | 2.9 |
| | 3-4 | CR-4 | 90 | 28 | 2.7 |
| | 3-5 | CR-5 | 90 | 26 | 2.6 |
| | 3-6 | CR-6 | 95 | 28 | 2.9 |
| | 3-7 | CR-7 | 90 | 27 | 2.8 |
| | 3-8 | CR-8 | 90 | 27 | 2.8 |
| | 3-9 | CR-9 | 90 | 28 | 2.7 |
| | 3-10 | CR-10 | 95 | 29 | 2.9 |
| | 3-11 | CR-11 | 95 | 27 | 2.8 |
| | 3-12 | CR-12 | 95 | 26 | 2.8 |
| | 3-13 | CR-13 | 90 | 28 | 3.1 |
| | 3-14 | CR-14 | 95 | 27 | 2.7 |
| | 3-15 | CR-15 | 95 | 29 | 2.8 |
| | 3-16 | CR-16 | 95 | 29 | 2.9 |
| | 3-17 | CR-17 | 95 | 28 | 2.8 |
| | 3-18 | CR-18 | 95 | 27 | 2.7 |
| | 3-19 | CR-19 | 90 | 27 | 2.8 |
| | 3-20 | CR-20 | 95 | 28 | 2.9 |
| | 3-21 | CR-21 | 95 | 27 | 2.8 |

It is demonstrated in Tables 5 and 6 that chemically amplified resist compositions comprising onium salts within the scope of the invention exhibit a high sensitivity and satisfactory CDU.

Japanese Patent Application No. 2024-090656 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An onium salt having the formula (1): 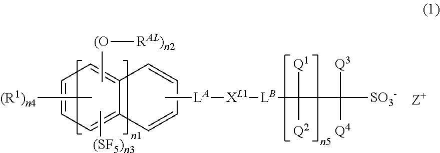
wherein n1 is 0 or 1, n2 is 1, 2, or 3, n3 is 1 or 2, n4 is 0, 1, or 2, n5 is 0, 1, 2, 3, or 4, provided that 2≤n2+n3+n4≤5 when n1 is 0, 2≤n2+n3+n4≤7 when n1 is 1,
R^{AL} is an acid labile group, at least one —SF₅ group and at least one-O—R^{AL} are bonded to adjacent carbon atoms,
R¹ is a halogen atom, a nitro group, a cyano group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n4 is 2, two R¹ may be identical or different and two R¹ may bond together to form a ring with the carbon atom to which they are attached,
L^{A} and L^{B} are each independently a single bond, an ether bond, an ester bond, a sulfonate ester bond, an amide bond, a sulfonamide bond, a carbonate bond, or a carbamate bond,
X^{L1} is a single bond or a C₁-C₄₀ hydrocarbylene group which may contain a heteroatom,
Q¹ and Q² are each independently a hydrogen atom, a fluorine atom, or a C₁-C₆ fluorinated saturated hydrocarbyl group,
Q³ and Q⁴ are each independently a fluorine atom or a C₁-C₆ fluorinated saturated hydrocarbyl group, and
Z⁺ is an onium cation.

2. The onium salt of claim 1 having the formula (1A): 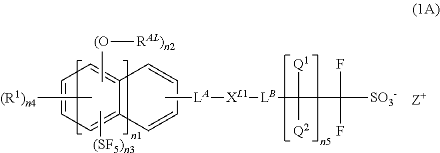
wherein n1 to n5, R^{AL}, R¹, L^{A}, L^{B}, X^{L1}, Q¹, Q², and Z⁺ are as defined above.

3. The onium salt of claim 2 having the formula (1B): 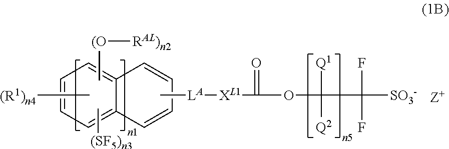
wherein n1 to n5, R^{AL}, R¹, L^{A}, X^{L1}, Q¹, Q², and Z⁺ are as defined above.

4. The onium salt of claim 1 wherein a structure of an acid labile group formed together with an adjacent oxygen atom has the formula (AL-1) or (AL-2): 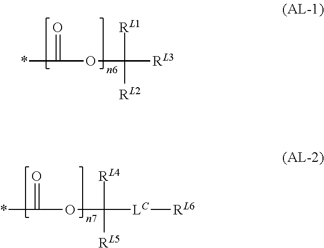
wherein n6 is 0 or 1, n7 is 0 or 1,
R^{L1}, R^{L2}, and R¹³ are each independently a C₁-C₁₂ hydrocarbyl group, a part of —CH₂—of the hydrocarbyl group may be substituted with —O—or —S—, when the hydrocarbyl group contains an aromatic ring, some or all of the hydrogen atoms of the aromatic ring may be substituted with a halogen atom, a cyano group, a nitro group, a C₁-C₄ alkyl group which may contain a halogen atom, or a C₁-C₄ alkoxy group which may contain a halogen atom, R^{L1} and R^{L2} may bond together to form a ring with the carbon atom to which they are attached, a part of —CH₂— of the ring may be substituted with —O—or —S—,
R^{L4} and R¹⁵ are each independently a hydrogen atom or a C₁-C₁₀ hydrocarbyl group, R^{L6} is a C₁-C₂₀ hydrocarbyl group, a part of —CH₂— of the hydrocarbyl group may be substituted with —O—or —S—, R^{L5} and R^{L6} may bond together to form a C₃-C₂₀ heterocyclic group with the carbon atom to which they are attached and L^{C}, a part of —CH₂— of the heterocyclic group may be substituted with —O—or —S—,
L^{C} is-O—or —S—, and
*designates a point of attachment to adjacent —O—.

5. The onium salt of claim 1 wherein Z⁺ is a sulfonium cation having the formula (Z-1) or an iodonium cation having the formula (Z-2): 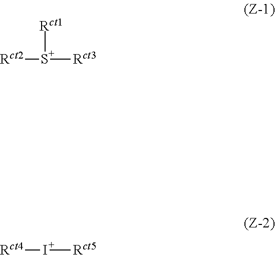
wherein R^{ct1} to R^{ct5} are each independently a halogen atom or a C₁-C₃₀ hydrocarbyl group which may contain a heteroatom, and R^{ct1} and R^{ct2} may bond together to form a ring with the sulfur atom to which they are attached.

6. The onium salt of claim 1 wherein Z⁺ is a sulfonium cation having the formula (Z-3): 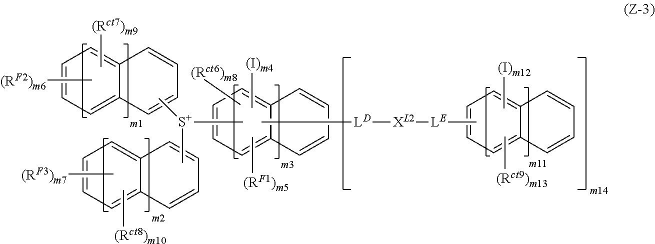
wherein m1 is 0 or 1, m2 is 0 or 1, m3 is 0 or 1, m4 is 0, 1, 2, 3, or 4, m5 is 0, 1, 2, 3, or 4, m6 is 0, 1, 2, 3, 4, 5, or 6, m7 is 0, 1, 2, 3, 4, 5, or 6, m8 is 0, 1, or 2, m9 is 0, 1, or 2, m10 is 0, 1, or 2, m11 is 0 or 1, m12 is 0, 1, 2, 3, or 4, m13 is 0, 1, or 2, m14 is 0, 1, or 2, provided that 0≤m6+m9≤4 when m1 is 0, 0≤m6+m9≤6 when m1 is 1, 0≤m7+m10≤4 when m2 is 0, 0≤m7+m10≤6 when m2 is 1, 1≤m4+m5+m8+m14≤4 when m3 is 0, 1≤m4+m5+m8+m14≤6 when m3 is 1, 0≤m12+m13≤4 when m11 is 0,0≤m12+m13≤6 when m11 is 1, m4+m12 ≥1,
R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, R^{F1} may be identical or different when m5 is 2 or more, R^{F2} may be identical or different when m6 is 2 or more, R^{F3} may be identical or different when m7 is 2 or more,
R^{ct6} to R^{ct9} are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m8 is 2, two R^{ct6} may be identical or different and two R^{ct6} may bond together to form a ring with the carbon atom to which they are attached, when m9 is 2, two R^{ct7} may be identical or different and two R^{ct7} may bond together to form a ring with the carbon atom to which they are attached, when m10 is 2, two R^{ct8} may be identical or different and two R^{ct8} may bond together to form a ring with the carbon atom to which they are attached, when m13 is 2, two R^{ct9} may be identical or different and two R^{ct9} may bond together to form a ring with the carbon atom to which they are attached,
aromatic rings which are directly bonded to S⁺ in the sulfonium cation may bond together to form a ring with S⁺,
L^{D} and L^{E} are each independently a single bond, an ether bond, an ester bond, an amide bond, a sulfonate ester bond, a sulfonamide bond, a carbonate bond, or a carbamate bond, and
X^{L2} is a single bond or a C₁-C₄₀ hydrocarbylene group which may contain a heteroatom.

7. A photoacid generator comprising the onium salt of claim 1.

8. A chemically amplified resist composition comprising the photoacid generator of claim 7.

9. The chemically amplified resist composition of claim 8 further comprising a base polymer comprising at least one selected from a repeat unit having the formula (a1), a repeat unit having the formula (a2), and a repeat unit having the formula (a3): 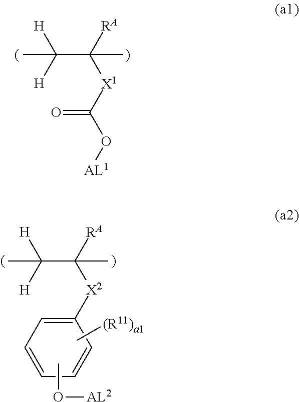
wherein R^{A} is each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, the phenylene group or the naphthylene group may be substituted with a hydroxy group, a nitro group, a cyano group, an optionally fluorinated C₁-C₁₀ saturated hydrocarbyl group, an optionally fluorinated C₁-C₁₀ saturated hydrocarbyloxy group, or a halogen atom, X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring,
X² is a single bond, *—C(═O)—O—, or *—C(═O)—NH—,
* designates a point of attachment to the carbon atom in the backbone,
R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, R¹¹ may be identical or different when a1 is 2, 3, or 4,
A^{L1} and A^{L2} are each independently an acid labile group, and
a1 is 0, 1, 2, 3, or 4, 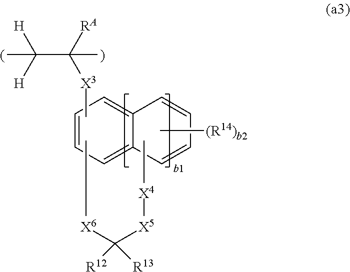
wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to the carbon atom in the backbone,
X⁴ is a single bond, a C₁-C₄ aliphatic hydrocarbylene group, a carbonyl group, a sulfonyl group, or a group obtained by combining the foregoing,
X⁵ and X⁶ are each independently an oxygen atom or a sulfur atom, provided that X⁴ and X⁶ are bonded to adjacent carbon atoms of the aromatic ring,
R¹² and R¹³ are each independently a hydrogen atom or a C₁-C₂₀ hydrocarbyl group which may contain a heteroatom, R¹² and R¹³ may bond together to form a ring with the carbon atom to which they are attached,
R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently a hydrogen atom or a C₁-C₆ hydrocarbyl group, and when b2 is 2 or more, R¹⁴ may be identical or different, and a plurality of R¹⁴ may bond together to form a ring with the carbon atom of the aromatic ring to which they are attached.

10. The chemically amplified resist composition of claim 9 wherein the base polymer comprises at least one selected from a repeat unit having the formula (b1) and a repeat unit having the formula (b2): 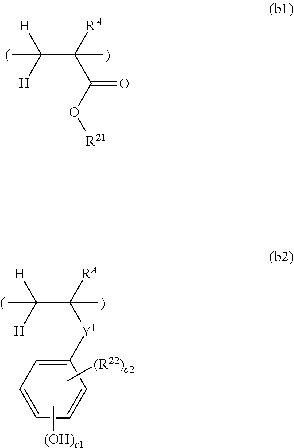
wherein R^{A} is each independently a hydrogen atom, a fluorine atom, a methyl group, or a trifluoromethyl group,
Y¹ is a single bond or *—C(═O)—O—, * designates a point of attachment to the carbon atom in the backbone,
R²¹ is a hydrogen atom or a C₁-C₂₀ group having at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—),
R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, R²² may be identical or different when c2 is 2, 3, or 4,
c1 is 1, 2, 3, or 4, and c2 is 0, 1, 2, 3, or 4, provided that 1≤c1+c2≤5.

11. The chemically amplified resist composition of claim 9 wherein the base polymer comprises at least one selected from a repeat unit having the formula (c1), a repeat unit having the formula (c2), a repeat unit having the formula (c3), a repeat unit having the formula (c4), and a repeat unit having the formula (c5): 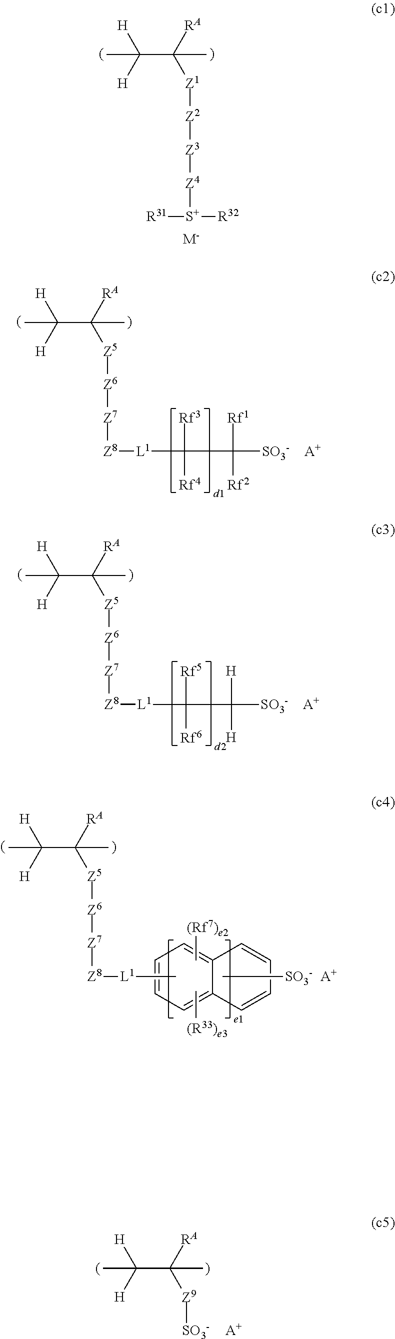
wherein d1 and d2 are each independently 0, 1, 2, or 3, e1 is 0 or 1, e2 is 0, 1, 2, 3, or 4, e3 is 0, 1, 2, 3, or 4, provided that 0≤e2+e3≤4 when e1 is 0, 0≤e2+e3≤6 when e1 is 1,
R^{A} is each independently a hydrogen atom, a fluorine atom, a methyl group, or a trifluoromethyl group,
Z¹ is a single bond or a phenylene group which may have a substituent,
Z² is a single bond, **—C(═O)—O—Z²¹—, **—C(═O)—NH—Z²¹—, or **—O—Z²¹—, Z²¹ is a C₁-C₆ aliphatic hydrocarbylene group, a phenylene group, or a divalent group obtained by combining the foregoing, and may contain a halogen atom, a carbonyl group, an ester bond, an ether bond, or a hydroxy group,
Z³ is a single bond, an ether bond, an ester bond, an amide bond, a sulfonate ester bond, a sulfonamide bond, a carbonate bond, or a carbamate bond,
Z⁴ is a single bond, or a C₁-C₆ aliphatic hydrocarbylene group, a phenylene group, or a divalent group obtained by combining the foregoing, and may contain a halogen atom, a carbonyl group, an ester bond, an ether bond, or a hydroxy group,
Z⁵ is each independently a single bond, a phenylene group which may have a substituent, a naphthylene group, or *—C(═O)—O—Z⁵¹—, Z⁵¹ is a C₁-C₁₀ aliphatic hydrocarbylene group, a phenylene group, or a naphthylene group, the aliphatic hydrocarbylene group may contain a halogen atom, a hydroxy group, an ether bond, an ester bond, or a lactone ring,
Z⁶ is a single bond, an ether bond, an ester bond, an amide bond, a sulfonate ester bond, a sulfonamide bond, a carbonate bond, or a carbamate bond,
Z⁷ is each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom,
Z⁸ is each independently a single bond, ****—Z⁸¹—C(—O)—O—, ****—C(═O)—NH—Z⁸¹—, or ****—O—Z⁸¹—, Z⁸¹ is a C₁-C₂₀ hydrocarbylene group which may contain a heteroatom,
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—N(H)—Z⁹¹—, or *—O—Z⁹¹—, Z⁹¹ is a phenylene group substituted with a C₁-C₆ aliphatic hydrocarbylene group, a phenylene group, a fluorinated phenylene group, or a trifluoromethyl group, and may contain a carbonyl group, an ester bond, an ether bond, or a hydroxy group,
designates a point of attachment to the carbon atom in the backbone, ** designates a point of attachment to Z¹, *** designates a point of attachment to Z⁶, **** designates a point of attachment to Z⁷,
L¹ is a single bond, an ether bond, an ester bond, a carbonyl group, a sulfonate ester bond, a sulfonamide bond, a carbonate bond, or a carbamate bond,
R^{f1} and R^{f2} are each independently a fluorine atom or a C₁-C₆ fluorinated saturated hydrocarbyl group,
R^{f3} and R^{f4} are each independently a hydrogen atom, a fluorine atom, or a C₁-C₆ fluorinated saturated hydrocarbyl group,
R^{f5} and R^{f6} are each independently a hydrogen atom, a fluorine atom, or a C₁-C₆ fluorinated saturated hydrocarbyl group, provided that not all R^{f5} and R^{f6} are hydrogen atoms at the same time,
Rf⁷ is a fluorine atom, a C₁-C₆ fluorinated alkyl group, a C₁-C₆ fluorinated alkoxy group, or a C₁-C₆ fluorinated alkylthio group,
R³¹ and R³² are each independently a C₁-C₂₀ hydrocarbyl group which may contain a heteroatom, R³¹ and R³² may bond together to form a ring with the sulfur atom to which they are attached,
R³³ is a halogen atom other than a fluorine atom, or a C₁-C₂₀ hydrocarbyl group which may contain a heteroatom, when e3 is 2, 3, or 4, R³³ may be identical or different, a plurality of R³³ may bond together to form a ring with the carbon atom to which they are attached,
M⁻ is a non-nucleophilic counter ion, and
A⁺ is an onium cation.

12. The chemically amplified resist composition of claim 8 further comprising an organic solvent.

13. The chemically amplified resist composition of claim 8 further comprising a quencher.

14. The chemically amplified resist composition of claim 8 further comprising a photoacid generator other than the photoacid generator.

15. The chemically amplified resist composition of claim 8 further comprising a surfactant.

16. A pattern forming process comprising the steps of forming a resist film on a substrate using the chemically amplified resist composition of claim 8, exposing the resist film to high-energy radiation, and developing the exposed resist film in a developer.

17. The pattern forming process of claim 16 wherein the high-energy radiation is KrF excimer laser radiation, ArF excimer laser radiation, EB, or EUV of wavelength 3 to 15 nm.
